SEQUENCE LISTING (228 sequences)

SEQ ID NO: 1 - protein, 12 aa, synthetic construct
GFSLSTSGMGVS

SEQ ID NO: 2 - protein, 16 aa, synthetic construct
HIYWDDDKRYNPSLKS

SEQ ID NO: 3 - protein, 11 aa, synthetic construct
RETVFYWYFDV

SEQ ID NO: 4 - protein, 11 aa, synthetic construct
WGRGTPVTVSS

SEQ ID NO: 5 - protein, 121 aa, synthetic construct
QVTLRESGPALVKPTQTLTLTCTFSGFSLSTSGMGVSWIRQPPGKGLEWLAHIYWDDDKRYNPSLKSRLTISKDTSRNQVVLTMTNMDPVDTATYYCARRETVFYWYFDVWGRGTPVTVSS

SEQ ID NO: 6 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 7 - protein, 15 aa, synthetic construct
EPKSCDKTHTCPPCP

SEQ ID NO: 8 - protein, 110 aa, synthetic construct
APEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 9 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 451 aa, synthetic construct
QVTLRESGPALVKPTQTLTLTCTFSGFSLSTSGMGVSWIRQPPGKGLEWLAHIYWDDDKRYNPSLKSRLTISKDTSRNQVVLTMTNMDPVDTATYYCARRETVFYWYFDVWGRGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 15 aa, synthetic construct
KASQSVDYDGDSYMN

SEQ ID NO: 12 - protein, 7 aa, synthetic construct
AASNLES

SEQ ID NO: 13 - protein, 9 aa, synthetic construct
QQSNEDPPR

SEQ ID NO: 14 - protein, 10 aa, synthetic construct
FGGGTKVEIK

SEQ ID NO: 15 - protein, 111 aa, synthetic construct
DIVMTQSPDSLAVSLGERATINCKASQSVDYDGDSYMNWYQQKPGQPPKLLIYAASNLESGVPDRFSGSGSGTDFTLTISSLQAEDVAVYYCQQSNEDPPRFGGGTKVEIK

SEQ ID NO: 16 - protein, 107 aa, synthetic construct [sequence omitted]

SEQ ID NO: 17 - protein, 218 aa, synthetic construct
DIVMTQSPDSLAVSLGERATINCKASQSVDYDGDSYMNWYQQKPGQPPKLLIYAASNLESGVPDRFSGSGSGTDFTLTISSLQAEDVAVYYCQQSNEDPPRFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 12 aa, synthetic construct
GFSLSNFGEGLS

SEQ ID NO: 19 - protein, 121 aa, synthetic construct
EVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGRGTPVTVSS

SEQ ID NO: 20 - protein, 111 aa, synthetic construct
DIQMTQSPSSLSASVGDRVTITCKASQSVDEDGDSYMNWYQQKPGKAPKLLIYAASNLESGVPSRFSGSGSGTDFTLTISSLQPEDFATYYCQQSNEDPPTFGGGTKVEIK

SEQ ID NO: 21 - protein, 11 aa, synthetic construct
WGQGTTVTVSS

SEQ ID NO: 22 - protein, 121 aa, synthetic construct
EVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSS

SEQ ID NO: 23 - protein, 451 aa, synthetic construct
EVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 15 aa, synthetic construct
RASQSVDEEGDSYMN

SEQ ID NO: 25 - protein, 9 aa, synthetic construct
QQSNKDPPT

SEQ ID NO: 26 - protein, 111 aa, synthetic construct
DIQMTQSPSSLSASVGDRVTITCRASQSVDEEGDSYMNWYQQKPGKAPKLLIYAASNLESGVPSRFSGSGSGTDFTLTISSLQPEDFATYYCQQSNKDPPTFGGGTKVEIK

SEQ ID NO: 27 - protein, 218 aa, synthetic construct
DIQMTQSPSSLSASVGDRVTITCRASQSVDEEGDSYMNWYQQKPGKAPKLLIYAASNLESGVPSRFSGSGSGTDFTLTISSLQPEDFATYYCQQSNKDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 121 aa, synthetic construct
EVTLRESGPALVKPTQTLTLTCTFSGFSLSNFGEGLSWIRQPPGKGLEWLAHIYWDDDKRYSTSLKTRLTISKDTSRNQVVLTMTNMDPVDTATYYCARRETVFYWYFDVWGQGTTVTVSS

SEQ ID NO: 29 - protein, 111 aa, synthetic construct
DIQMTQSPSSLSASVGDRVTITCRASQSVDEEGDSYMNWYQQKPGKAPKLLIYAASNLESGVPSRFSGSGSGTDFTLTISSLQPEDFATYYCQQSHKDPPTFGGGTKVEIK

SEQ ID NO: 30 - protein, 111 aa, synthetic construct
DIQMTQSPSSLSASVGDRVTITCRASQSVDEEGDSYMNWYQQKPGKAPKLLIYAASNLESGVPSRFSGSGSGTDFTLTISSLQPEDFATYYCQQSNEDPPTFGGGTKVEIK

SEQ ID NO: 31 - protein, 10 aa, synthetic construct
GFTFISYAMS

SEQ ID NO: 32 - protein, 16 aa, synthetic construct
SISSGGNTYYPDSVKG

SEQ ID NO: 33 - protein, 10 aa, synthetic construct
LDGYYFGFAY

SEQ ID NO: 34 - protein, 118 aa, synthetic construct
EVQLVESGGGLVQPGGSLRLSCAASGFTFISYAMSWVRQAPGKGLEWVASISSGGNTYYPDSVKGRFTISRDNAKNSLYLQMNSLRAEDTAVYYCARLDGYYFGFAYWGQGTLVTVSS

SEQ ID NO: 35 - protein, 448 aa, synthetic construct
EVQLVESGGGLVQPGGSLRLSCAASGFTFISYAMSWVRQAPGKGLEWVASISSGGNTYYPDSVKGRFTISRDNAKNSLYLQMNSLRAEDTAVYYCARLDGYYFGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 15 aa, synthetic construct
KASESVDNYGKSLMH

SEQ ID NO: 37 - protein, 7 aa, synthetic construct
RASNLES

SEQ ID NO: 38 - protein, 9 aa, synthetic construct
QQSNEDPWT

SEQ ID NO: 39 - protein, 111 aa, synthetic construct
DIQMTQSPSSLSASVGDRVTITCKASESVDNYGKSLMHWYQQKPGKAPKLLIYRASNLESGVPSRFSGSGSGTDFTLTISSLQPEDFATYYCQQSNEDPWTFGGGTKVEIK

SEQ ID NO: 40 - protein, 218 aa, synthetic construct
DIQMTQSPSSLSASVGDRVTITCKASESVDNYGKSLMHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 10 aa, synthetic construct
GFTFSSYAMS

SEQ ID NO: 42 - protein, 16 aa, synthetic construct
SISSGDTTYYPDSVKG

SEQ ID NO: 43 - protein, 10 aa, synthetic construct
LDGYYFGFPY

SEQ ID NO: 44 - protein, 118 aa, synthetic construct
EVKLVESGGDLVKPGGSLKLSCAASGFTFSSYAMSWVRQSPDKRLEWVASISSGDTTYYPDSVKGRFTISRDNARNILYLQMTSLRSEDTAMYYCARLDGYYFGFPYWGQGTLVTVSA

SEQ ID NO: 45 - protein, 15 aa, synthetic construct
KASESVDHSGSSLMH

SEQ ID NO: 46 - protein, 111 aa, synthetic construct
DTVLTQSPASLAVSLGQRATISCKASESVDHSGSSLMHWYQQKPGQSPKLLIYRASNLESGIPARFSGSGSRTDFTLTINPVEADDVATYYCQQSNEDPWTFGGGTKLEIK

SEQ ID NO: 47 - protein, 11 aa, synthetic construct
WGQGTLVTVSS

SEQ ID NO: 48 - protein, 118 aa, synthetic construct
EVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARLDGYYFGFPYWGQGTLVTVSS

SEQ ID NO: 49 - protein, 111 aa, synthetic construct
DIQMTQSPSSLSASVGDRVTITCKASESVDHSGSSLMHWYQQKPGKAPKLLIYRASNLESGVPSRFSGSGSGTDFTLTISSLQPEDFATYYCQQSNEDPWTFGGGTKVEIK

SEQ ID NO: 50 - protein, 10 aa, synthetic construct
NEGYYFGLTL

SEQ ID NO: 51 - protein, 118 aa, synthetic construct
EVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSS

SEQ ID NO: 52 - protein, 448 aa, synthetic construct
EVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 15 aa, synthetic construct
KASESVDHFGWSLVH

SEQ ID NO: 54 - protein, 111 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIK

SEQ ID NO: 55 - protein, 218 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 11 aa, synthetic construct
WGRGTLVTVSS

SEQ ID NO: 57 - protein, 118 aa, synthetic construct
EVQLVESGGGLVQPGGSLRLSCAASGFTFSSYAMSWVRQAPGKGLEWVASISTGDTTYYPDSVKGRFTISRDNAKNSLYLQMNSLRAEDTAVYYCARNEGYYFGLTQWGRGTLVTVSS

SEQ ID NO: 58 - protein, 111 aa, synthetic construct
DIQMTQSPSSLSASVGDRVTITCRASQSVDHFGWSLVHWYQQKPGKAPKLLIYRASNLESGVPSRFSGSGSGTDFTLTISSLQPEDFATYYCQQSNEDPWTFGGGTKVEIK

SEQ ID NO: 59 - protein, 111 aa, synthetic construct
DIQMTQSPSSLSASVGDRVTITCRASESVDHFGWSLVHWYQQKPGKAPKLLIYRADSLETGVPSRFSGSGSGTDFTLTISSLQPEDFATYYCQQSNEDPWTFGGGTKVEIK

SEQ ID NO: 60 - protein, 10 aa, synthetic construct
GFTFSSYWMY

SEQ ID NO: 61 - protein, 17 aa, synthetic construct
AITPNAGEDYYPESVKG

SEQ ID NO: 62 - protein, 12 aa, synthetic construct
GHYYYTSYSLGY

SEQ ID NO: 63 - protein, 121 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGHYYYTSYSLGYWGQGTLVTVSS

SEQ ID NO: 64 - protein, 451 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGHYYYTSYSLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 11 aa, synthetic construct
KASQNINKHLD

SEQ ID NO: 66 - protein, 7 aa, synthetic construct
FTNNLQT

SEQ ID NO: 67 - protein, 8 aa, synthetic construct
FQYNQGWT

SEQ ID NO: 68 - protein, 106 aa, synthetic construct
DIQMTQSPSSLSASVGDRVTITCKASQNINKHLDWYQQKPGKAPKLLIYFTNNLQTGVPSRFSGSGSGTDFTLTISSLQPEDFATYYCFQYNQGWTFGGGTKVEIK

SEQ ID NO: 69 - protein, 213 aa, synthetic construct
DIQMTQSPSSLSASVGDRVTITCKASQNINKHLDWYQQKPGKAPKLLIYFTNNLQTGVPSRFSGSGSGTDFTLTISSLQPEDFATYYCFQYNQGWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 11 aa, synthetic construct
RASQPISKHLD

SEQ ID NO: 71 - protein, 106 aa, synthetic construct
DIQMTQSPSSLSASVGDRVTITCRASQPISKHLDWYQQKPGKAPKLLIYFTNNLQTGVPSRFSGSGSGTDFTLTISSLQPEDFATYYCFQYNQGWTFGGGTKVEIK

SEQ ID NO: 72 - protein, 12 aa, synthetic construct
GQYYYTKYSLGY

SEQ ID NO: 73 - protein, 121 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGQYYYTKYSLGYWGQGTLVTVSS

SEQ ID NO: 74 - protein, 451 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGQYYYTKYSLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 11 aa, synthetic construct
RASQPIHNHLD

SEQ ID NO: 76 - protein, 7 aa, synthetic construct
FGKNLQE

SEQ ID NO: 77 - protein, 8 aa, synthetic construct
FQYKKGWS

SEQ ID NO: 78 - protein, 106 aa, synthetic construct
DIQMTQSPSSLSASVGDRVTITCRASQPIHNHLDWYQQKPGKAPKLLIYFGKNLQEGVPSRFSGSGSGTDFTLTISSLQPEDFATYYCFQYKKGWSFGGGTKVEIK

SEQ ID NO: 79 - protein, 213 aa, synthetic construct
DIQMTQSPSSLSASVGDRVTITCRASQPIHNHLDWYQQKPGKAPKLLIYFGKNLQEGVPSRFSGSGSGTDFTLTISSLQPEDFATYYCFQYKKGWSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 121 aa, synthetic construct
EVQLVESGGGLVQPGGSLRLSCAASGFEFSHYWMYWVRQAPGKGLEWVAAITPNAGEDYYPESVKGRFTISRDNAKNSLYLQMNSLRAEDTAVYYCARGYYRYTKYSLGYWGQGTLVTVSS

SEQ ID NO: 81 - protein, 106 aa, synthetic construct
DIQMTQSPSSLSASVGDRVTITCRASQPIHQFLDWYQQKPGKAPKLLIYFGHILQEGVPSRFSGSGSGTDFTLTISSLQPEDFATYYCFQYKQGWSFGGGTKVEIK

SEQ ID NO: 82 - protein, 10 aa, synthetic construct
GFTFRTYGMH

SEQ ID NO: 83 - protein, 17 aa, synthetic construct
VIWYDGSNKHYADSVKG

SEQ ID NO: 84 - protein, 13 aa, synthetic construct
APQWELVHEAFDI

SEQ ID NO: 85 - protein, 13 aa, synthetic construct
APQWYLVHEAFDI

SEQ ID NO: 86 - protein, 11 aa, synthetic construct
GGNNLGSKSVH

SEQ ID NO: 87 - protein, 7 aa, synthetic construct
DDSDRPS

SEQ ID NO: 88 - protein, 7 aa, synthetic construct
DDKDRPS

SEQ ID NO: 89 - protein, 11 aa, synthetic construct
QVWDSSSDHVV

SEQ ID NO: 90 - protein, 11 aa, synthetic construct
QVWDSKSDHVV

SEQ ID NO: 91 - protein, 122 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 92 - protein, 122 aa, synthetic construct
QMQLVESGGGVVQPGRSLRLSCAASGFTFRTYGMHWVRQAPGKGLEWVAVIWYDGSNKHYADSVKGRFTISRDNSKNTLYLQMNSLRAEDTAVYYCARAPQWYLVHEAFDIWGQGTMVTVSS

SEQ ID NO: 93 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 94 - protein, 108 aa, synthetic construct
SYVLTQPPSVSVAPGQTARITCGGNNLGSKSVHWYQQKPGQAPVLVVYDDKDRPSWIPERFSGSNSGNTATLTISRGEAGDEADYYCQVWDSKSDHVVFGGGTKLTVL

SEQ ID NO: 95 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 96 - protein, 452 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 452 aa, synthetic construct
QMQLVESGGGVVQPGRSLRLSCAASGFTFRTYGMHWVRQAPGKGLEWVAVIWYDGSNKHYADSVKGRFTISRDNSKNTLYLQMNSLRAEDTAVYYCARAPQWYLVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 214 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - protein, 214 aa, synthetic construct
SYVLTQPPSVSVAPGQTARITCGGNNLGSKSVHWYQQKPGQAPVLVVYDDKDRPSWIPERFSGSNSGNTATLTISRGEAGDEADYYCQVWDSKSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 100 - protein, 11 aa, synthetic construct
WGQGTMVTVSS

SEQ ID NO: 101 - protein, 10 aa, synthetic construct
FGGGTKLTVL

SEQ ID NO: 102 - protein, 5 aa, synthetic construct
EPKSC

SEQ ID NO: 103 - protein, 224 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGHYYYTSYSLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 104 - protein, 5 aa, synthetic construct
GGGGS

SEQ ID NO: 105 - protein, 98 aa, synthetic construct
ASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKV

SEQ ID NO: 106 - protein, 107 aa, synthetic construct
GQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 107 - protein, 669 aa, synthetic construct
DIQMTQSPSSLSASVGDRVTITCKASQNINKHLDWYQQKPGKAPKLLIYFTNNLQTGVPSRFSGSGSGTDFTLTISSLQPEDFATYYCFQYNQGWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 108 - protein, 107 aa, synthetic construct
RTVAAPSVFIFPPSDEQLKSGTASVSCLLNNFYPREAKVQWKVDNALQSGNSQESVTEQDSKDSTYSLKSTLTLSKADYEKHKVYACEVTHQGLSSPVTKSFNRGEC

SEQ ID NO: 109 - protein, 218 aa, synthetic construct
DIQMTQSPSSLSASVGDRVTITCKASQSVDEDGDSYMNWYQQKPGKAPKLLIYAASNLESGVPSRFSGSGSGTDFTLTISSLQPEDFATYYCQQSNEDPPTFGGGTKVEIKRTVAAPSVFIFPPSDEQLKSGTASVSCLLNNFYPREAKVQWKVDNALQSGNSQESVTEQDSKDSTYSLKSTLTLSKADYEKHKVYACEVTHQGLSSPVTKSFNRGEC

SEQ ID NO: 110 - protein, 98 aa, synthetic construct
ASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKV

SEQ ID NO: 111 - protein, 107 aa, synthetic construct
GQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 448 aa, synthetic construct
EVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 107 aa, synthetic construct
RTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 114 - protein, 107 aa, synthetic construct
GQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 115 - protein, 669 aa, synthetic construct
DIQMTQSPSSLSASVGDRVTITCRASQPISKHLDWYQQKPGKAPKLLIYFTNNLQTGVPSRFSGSGSGTDFTLTISSLQPEDFATYYCFQYNQGWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYSTSLKTRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 116 - protein, 218 aa, synthetic construct
DIQMTQSPSSLSASVGDRVTITCRASQSVDEEGDSYMNWYQQKPGKAPKLLIYAASNLESGVPSRFSGSGSGTDFTLTISSLQPEDFATYYCQQSHKDPPTFGGGTKVEIKRTVAAPSVFIFPPSDEQLKSGTASVSCLLNNFYPREAKVQWKVDNALQSGNSQESVTEQDSKDSTYSLKSTLTLSKADYEKHKVYACEVTHQGLSSPVTKSFNRGEC

SEQ ID NO: 117 - protein, 107 aa, synthetic construct
GQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 118 - protein, 448 aa, synthetic construct
EVQLVESGGGLVQPGGSLRLSCAASGFTFSSYAMSWVRQAPGKGLEWVASISTGDTTYYPDSVKGRFTISRDNAKNSLYLQMNSLRAEDTAVYYCARNEGYYFGLTQWGRGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 119 - protein, 218 aa, synthetic construct
DIQMTQSPSSLSASVGDRVTITCRASQSVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 120 - protein, 218 aa, synthetic construct
DIQMTQSPSSLSASVGDRVTITCRASESVDHFGWSLVHWYQQKPGKAPKLLIYRADSLETGVPSRFSGSGSGTDFTLTISSLQPEDFATYYCQQSNEDPWTFGGGTKVEIKRTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 121 - protein, 669 aa, synthetic construct
DIQMTQSPSSLSASVGDRVTITCKASQNINKHLDWYQQKPGKAPKLLIYFTNNLQTGVPSRFSGSGSGTDFTLTISSLQPEDFATYYCFQYNQGWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 122 - protein, 221 aa, synthetic construct
EVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 123 - protein, 15 aa, synthetic construct
EPKSCEKTHTCPECP

SEQ ID NO: 124 - protein, 107 aa, synthetic construct
GQPREPQVYTLPPSREEMTKNQVSLTCEVKGFYPSDIAVEWESNGQPENNYKTTPPVLDSDGSFFLYSKLTVDKSRWQQGNVFSCSVLHEALHSHYTQKSLSLSPGK

SEQ ID NO: 125 - protein, 674 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPECPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVLHEALHSHYTQKSLSLSPGK

SEQ ID NO: 126 - protein, 15 aa, synthetic construct
EPKSCRKTHTCPRCP

SEQ ID NO: 127 - protein, 107 aa, synthetic construct
GQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 128 - protein, 451 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGQYYYTKYSLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPRCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 129 - protein, 15 aa, synthetic construct
EPKSCEKTHTCPPCP

SEQ ID NO: 130 - protein, 674 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVLHEALHSHYTQKSLSLSPGK

SEQ ID NO: 131 - protein, 15 aa, synthetic construct
EPKSCRKTHTCPPCP

SEQ ID NO: 132 - protein, 451 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGQYYYTKYSLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 133 - protein, 674 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVLHEALHSHYTQKSLSLSPGK

SEQ ID NO: 134 - protein, 451 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGQYYYTKYSLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 135 - protein, 674 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 136 - protein, 218 aa, synthetic construct
DIQMTQSPSSLSASVGDRVTITCRASQSVDEEGDSYMNWYQQKPGKAPKLLIYAASNLESGVPSRFSGSGSGTDFTLTISSLQPEDFATYYCQQSNKDPPTFGGGTKVEIKRTVAAPSVFIFPPSDEQLKSGTASVSCLLNNFYPREAKVQWKVDNALQSGNSQESVTEQDSKDSTYSLKSTLTLSKADYEKHKVYACEVTHQGLSSPVTKSFNRGEC

SEQ ID NO: 137 - protein, 451 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGQYYYTKYSLGYWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 138 - protein, 213 aa, synthetic construct
DIQMTQSPSSLSASVGDRVTITCRASQPIHNHLDWYQQKPGKAPKLLIYFGKNLQEGVPSRFSGSGSGTDFTLTISSLQPEDFATYYCFQYKKGWSFGGGTKVEIKRTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 139 - protein, 107 aa, synthetic construct
GQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 140 - protein, 674 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 141 - protein, 107 aa, synthetic construct
GQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 142 - protein, 451 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGQYYYTKYSLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 143 - protein, 224 aa, synthetic construct
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGQYYYTKYSLGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 144 - protein, 669 aa, synthetic construct
DIQMTQSPSSLSASVGDRVTITCRASQPIHNHLDWYQQKPGKAPKLLIYFGKNLQEGVPSRFSGSGSGTDFTLTISSLQPEDFATYYCFQYKKGWSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 145 - protein, 666 aa, synthetic construct
DIQMTQSPSSLSASVGDRVTITCKASQNINKHLDWYQQKPGKAPKLLIYFTNNLQTGVPSRFSGSGSGTDFTLTISSLQPEDFATYYCFQYNQGWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 451 aa, synthetic construct
EVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 147 - protein, 106 aa, synthetic construct
QPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 148 - protein, 106 aa, synthetic construct
QPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 681 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 211 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 151 - protein, 684 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 152 - protein, 678 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 681 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 154 - protein, 664 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 228 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 156 - protein, 667 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 157 - protein, 670 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 158 - protein, 225 aa, synthetic construct
QMQLVESGGGVVQPGRSLRLSCAASGFTFRTYGMHWVRQAPGKGLEWVAVIWYDGSNKHYADSVKGRFTISRDNSKNTLYLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 159 - protein, 684 aa, synthetic construct
QMQLVESGGGVVQPGRSLRLSCAASGFTFRTYGMHWVRQAPGKGLEWVAVIWYDGSNKHYADSVKGRFTISRDNSKNTLYLQMNSLRAEDTAVYYCARAPQWELVHEAFDIWGQGTMVTVSSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 160 - protein, 667 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 452 aa, synthetic construct
QMQLVESGGGVVQPGRSLRLSCAASGFTFRTYGMHWVRQAPGKGLEWVAVIWYDGSNKHYADSVKGRFTISRDNSKNTLYLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPRCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 162 - protein, 681 aa, synthetic construct
EVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPECPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 214 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 164 - protein, 674 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPECPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 452 aa, synthetic construct
QMQLVESGGGVVQPGRSLRLSCAASGFTFRTYGMHWVRQAPGKGLEWVAVIWYDGSNKHYADSVKGRFTISRDNSKNTLYLQMNSLRAEDTAVYYCARAPQWYLVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 166 - protein, 10 aa, synthetic construct
GYSFTTYWLG

SEQ ID NO: 167 - protein, 17 aa, synthetic construct
IMSPVDSDIRYSPSFQG

SEQ ID NO: 168 - protein, 10 aa, synthetic construct
RRPGQGYFDF

SEQ ID NO: 169 - protein, 119 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 170 - protein, 448 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 11 aa, synthetic construct
RASQGISSWLA

SEQ ID NO: 172 - protein, 7 aa, synthetic construct
AASSLQS

SEQ ID NO: 173 - protein, 9 aa, synthetic construct
QQYNIYPYT

SEQ ID NO: 174 - protein, 10 aa, synthetic construct
FGQGTKLEIK

SEQ ID NO: 175 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 176 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 222 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 178 - protein, 667 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 670 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 180 - protein, 674 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYNPSLKSRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 181 - protein, 449 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 213 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 183 - protein, 670 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVTLRESGPALVKPTQTLTLTCTFSGFSLSNFGEGLSWIRQPPGKGLEWLAHIYWDDDKRYSTSLKTRLTISKDTSRNQVVLTMTNMDPVDTATYYCARRETVFYWYFDVWGQGTTVTVSSASTKGPSVFPEAPSSKSTSGGTAALGCLVKDYFPEPVTVSWNSGALTSGVHTFPAVLQSSGLYSLGSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 184 - protein, 218 aa, synthetic construct; identical to SEQ ID NO: 120

SEQ ID NO: 185 - protein, 449 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPRCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 186 - protein, 449 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSPGK

SEQ ID NO: 187 - DNA, 663 bp, synthetic construct
  source 1..663: mol_type = other DNA
gaggtgcagctggtggagtcagggggaggcttggtgcaacctggagggtccctgagactctcctgtgcagcctctggattcactttcagtagctatgccatgtcttgggttcgtcaggctccagggaaggggctggagtgggtcgcatccattagtagtggtgacaccacctactatccagacagcgtgaagggccgattcaccatctccagagataatgccaagaacagcctgtacctgcaaatgaacagtctgagggctgaggacaccgccgtgtattactgtgcacgaaacgagggttactacttcggactgaccctgtggggccaagggaccctggtcaccgtctcctctgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgt

SEQ ID NO: 188 - DNA, 2022 bp, synthetic construct
  source 1..2022: mol_type = other DNA
gacatccagatgacccagtctccatcctccctgtctgcatctgtgggagacagagtcaccatcacttgcaaagccagtgaaagtgttgatcactttggttggtctctcgtgcactggtatcagcagaaaccagggaaagctcctaagctcctgatctatcgtgcatccaacctggaatctggcgtcccatcaaggttcagtggcagtggatctggcacagatttcactctcaccatcagcagtctgcaacctgaagattttgcaacttactactgtcagcaaagtaatgaagatccctggaccttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggtggcgggtccgaggtcacactgagagagtccggccctgccctggtgaaacccacccagaccctgaccctgacatgcaccttctccggcttctccctgtccaacttcggcgagggcctatcctggattcggcagcctcctggcaagggcctggaatggctggcccacatctactgggacgacgacaagcggtacaaccccagcctgaagtcccggctgaccatctccaaggacacctcccggaaccaggtggtgctgaccatgaccaacatggaccccgtggacaccgccacctactactgcgccagacgggaaaccgtgttctactggtacttcgacgtgtggggccagggcaccaccgtgaccgtgtcctctgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgagaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaagagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggaaaa

SEQ ID NO: 189 - DNA, 654 bp, synthetic construct
  source 1..654: mol_type = other DNA
gacatccagatgacccagtctccatcctccctgtctgcatctgtgggagacagagtcaccatcacttgccgggcctcccagtccgtggacgaagagggcgactcctacatgaactggtatcagcagaaaccagggaaagctcctaagctcctgatctatgccgcctccaacctggaatccggcgtcccatcaaggttcagtggcagtggatctggcacagatttcactctcaccatcagcagtctgcaacctgaagattttgcaacttactactgtcagcagtccaacaaggacccccccaccttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 190 - DNA, 1353 bp, synthetic construct
  source 1..1353: mol_type = other DNA
gaggtgcagctggtggagtctggcggcggactggtgcagcctggcggctctctgagactgtcttgtgccgcctccggcttcaccttcagttcctactggatgtactgggtgaggcaggcccctggcaagggcctggagtgggtggccgccattactcctaatgccggtgaggactactatccagagtctgtgaaaggccggttcaccatctccagggacaacgccaagaactccctgtacctccagatgaactccctgagggccgaggataccgccgtgtactactgtgccagaggccagtattactataccaagtattcccttggatactggggccagggcaccctggtgaccgtgtcctctgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtcggaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaggctcaccgtggacaagagcaggtggcagcaggggaacgtcttctcatgctccgtgctgcatgaggctctgcacagccactacacgcagaagagcctctccctgtccccgggaaaa

SEQ ID NO: 191 - DNA, 639 bp, synthetic construct
  source 1..639: mol_type = other DNA
gacatccagatgacccagtccccctcttctctgtctgcctctgtgggcgacagagtgaccatcacctgtagagcaagtcagcctattcacaaccacttggactggtatcagcagaagcctggcaaggctcccaagctgctgatctactttggaaagaatttgcaagaaggcgtgccttccagattctccggctctggctctggcaccgatttcaccctgaccatctcctccctccagcctgaggatttcgccacctactactgctttcagtataagaaggggtggagctttggcggcggaacaaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 192 - DNA, 1356 bp, synthetic construct
  source 1..1356: mol_type = other DNA
cagatgcagctggtggagtctgggggaggcgtggtccagcctgggaggtccctgagactctcctgtgcagcgtctggattcaccttcagaacctatggcatgcactgggtccgccaggctccaggcaaggggctggagtgggtggcagttatatggtatgatggaagtaataaacactatgcagactccgtgaagggccgattcaccatctccagagacaattccaagaacacgctgtatctgcaaatgaacagcctgagagccgaggacacggctgtgtattactgtgcgagagcccctcagtggtacctcgttcatgaggcttttgatatctggggccaagggacaatggtcaccgtctcttcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtcggaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaggctcaccgtggacaagagcaggtggcagcaggggaacgtcttctcatgctccgtgctgcatgaggctctgcacagccactacacgcagaagagcctctccctgtccccgggaaaa

SEQ ID NO: 193 - DNA, 642 bp, synthetic construct
  source 1..642: mol_type = other DNA
tcctatgtgctgactcagccaccctccgtgtcagtggccccaggacagacagccaggattacctgtgggggaaacaaccttggaagtaaaagtgtgcactggtaccagcagaagccaggccaggcccctgtgctggtcgtctatgatgataaggaccggccctcatggattcctgagcgattctctggctccaactctgggaacaccgccaccctgaccatcagcaggggcgaagccggggatgaggccgactattactgtcaggtgtgggatagtaagagtgatcatgtggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 194 - DNA, 1347 bp, synthetic construct
  source 1..1347: mol_type = other DNA
gaggtgcagctggtgcagtctggagcagaggtgaaaaagcccggggagtctctgaagatctcctgtaagggttctggatacagctttaccacctactggctgggctgggtgcgccagatgcccgggaaaggcctggattggatagggatcatgagccctgtggactctgatattagatacagcccctccttccaaggccaggtcaccatgtcagtggacaagtccatcaccaccgcctacctgcagtggaatagcctgaaggcctccgacaccgccatgtattactgtgccagacgacgccctggtcaaggttatttcgatttttgggggcaaggaacattggtgaccgtcagttcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtcggaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaggctcaccgtggacaagagcaggtggcagcaggggaacgtcttctcatgctccgtgctgcatgaggctctgcacagccactacacgcagaagagcctctccctgtccccgggaaaa

SEQ ID NO: 195 - DNA, 642 bp, synthetic construct
  source 1..642: mol_type = other DNA
gacatccagatgacccagtctccatcctcactgtctgcatctgtgggagacagagtcaccatcacttgtcgggccagtcagggtattagcagctggctggcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatatttatccctacaccttcggacaaggtacaaaactggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 196 - protein, 218 aa, synthetic construct
DIQMTQSPSSLSASVGDRVTITCKASESVDHFGWSLVHWYQQKPGKAPKLLIYRASNLESGVPSRFSGSGSGTDFTLTISSLQPEDFATYYCQQSNEDPWTFGGGTKVEIKRTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 197 - protein, 218 aa, synthetic construct
DIQMTQSPSSLSASVGDRVTITCKASQSVDEDGDSYMNWYQQKPGKAPKLLIYAASNLESGVPSRFSGSGSGTDFTLTISSLQPEDFATYYCQQSNEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - DNA, 354 bp, synthetic construct
  source 1..354: mol_type = other DNA
gaggtgcagctggtggagtcagggggaggcttggtgcaacctggagggtccctgagactctcctgtgcagcctctggattcactttcagtagctatgccatgtcttgggttcgtcaggctccagggaaggggctggagtgggtcgcatccattagtagtggtgacaccacctactatccagacagcgtgaagggccgattcaccatctccagagataatgccaagaacagcctgtacctgcaaatgaacagtctgagggctgaggacaccgccgtgtattactgtgcacgaaacgagggttactacttcggactgaccctgtggggccaagggaccctggtcaccgtctcctct

SEQ ID NO: 199 - DNA, 333 bp, synthetic construct
  source 1..333: mol_type = other DNA
gacatccagatgacccagtctccatcctccctgtctgcatctgtgggagacagagtcaccatcacttgcaaagccagtgaaagtgttgatcactttggttggtctctcgtgcactggtatcagcagaaaccagggaaagctcctaagctcctgatctatcgtgcatccaacctggaatctggcgtcccatcaaggttcagtggcagtggatctggcacagatttcactctcaccatcagcagtctgcaacctgaagattttgcaacttactactgtcagcaaagtaatgaagatccctggaccttcggcggagggaccaaggtggagatcaaa

SEQ ID NO: 200 - DNA, 363 bp, synthetic construct
  source 1..363: mol_type = other DNA
gaggtcacactgagagagtccggccctgccctggtgaaacccacccagaccctgaccctgacatgcaccttctccggcttctccctgtccaacttcggcgagggcctatcctggattcggcagcctcctggcaagggcctggaatggctggcccacatctactgggacgacgacaagcggtacaaccccagcctgaagtcccggctgaccatctccaaggacacctcccggaaccaggtggtgctgaccatgaccaacatggaccccgtggacaccgccacctactactgcgccagacgggaaaccgtgttctactggtacttcgacgtgtggggccagggcaccaccgtgaccgtgtcctct

SEQ ID NO: 201 - DNA, 333 bp, synthetic construct
  source 1..333: mol_type = other DNA
gacatccagatgacccagtctccatcctccctgtctgcatctgtgggagacagagtcaccatcacttgccgggcctcccagtccgtggacgaagagggcgactcctacatgaactggtatcagcagaaaccagggaaagctcctaagctcctgatctatgccgcctccaacctggaatccggcgtcccatcaaggttcagtggcagtggatctggcacagatttcactctcaccatcagcagtctgcaacctgaagattttgcaacttactactgtcagcagtccaacaaggacccccccaccttcggcggagggaccaaggtggagatcaaa

SEQ ID NO: 202 - DNA, 363 bp, synthetic construct
  source 1..363: mol_type = other DNA
gaggtgcagctggtggagtctggcggcggactggtgcagcctggcggctctctgagactgtcttgtgccgcctccggcttcaccttcagttcctactggatgtactgggtgaggcaggcccctggcaagggcctggagtgggtggccgccattactcctaatgccggtgaggactactatccagagtctgtgaaaggccggttcaccatctccagggacaacgccaagaactccctgtacctccagatgaactccctgagggccgaggataccgccgtgtactactgtgccagaggccagtattactataccaagtattcccttggatactggggccagggcaccctggtgaccgtgtcctct

SEQ ID NO: 203 - DNA, 318 bp, synthetic construct
  source 1..318: mol_type = other DNA
gacatccagatgacccagtccccctcttctctgtctgcctctgtgggcgacagagtgaccatcacctgtagagcaagtcagcctattcacaaccacttggactggtatcagcagaagcctggcaaggctcccaagctgctgatctactttggaaagaatttgcaagaaggcgtgccttccagattctccggctctggctctggcaccgatttcaccctgaccatctcctccctccagcctgaggatttcgccacctactactgctttcagtataagaaggggtggagctttggcggcggaacaaaggtggagatcaag

SEQ ID NO: 204 - DNA, 366 bp, synthetic construct
  source 1..366: mol_type = other DNA
cagatgcagctggtggagtctgggggaggcgtggtccagcctgggaggtccctgagactctcctgtgcagcgtctggattcaccttcagaacctatggcatgcactgggtccgccaggctccaggcaaggggctggagtgggtggcagttatatggtatgatggaagtaataaacactatgcagactccgtgaagggccgattcaccatctccagagacaattccaagaacacgctgtatctgcaaatgaacagcctgagagccgaggacacggctgtgtattactgtgcgagagcccctcagtggtacctcgttcatgaggcttttgatatctggggccaagggacaatggtcaccgtctcttca

SEQ ID NO: 205 - DNA, 324 bp, synthetic construct
  source 1..324: mol_type = other DNA
tcctatgtgctgactcagccaccctccgtgtcagtggccccaggacagacagccaggattacctgtgggggaaacaaccttggaagtaaaagtgtgcactggtaccagcagaagccaggccaggcccctgtgctggtcgtctatgatgataaggaccggccctcatggattcctgagcgattctctggctccaactctgggaacaccgccaccctgaccatcagcaggggcgaagccggggatgaggccgactattactgtcaggtgtgggatagtaagagtgatcatgtggtattcggcggagggaccaagctgaccgtccta

SEQ ID NO: 206 - DNA, 357 bp, synthetic construct
  source 1..357: mol_type = other DNA
gaggtgcagctggtgcagtctggagcagaggtgaaaaagcccggggagtctctgaagatctcctgtaagggttctggatacagctttaccacctactggctgggctgggtgcgccagatgcccgggaaaggcctggattggatagggatcatgagccctgtggactctgatattagatacagcccctccttccaaggccaggtcaccatgtcagtggacaagtccatcaccaccgcctacctgcagtggaatagcctgaaggcctccgacaccgccatgtattactgtgccagacgacgccctggtcaaggttatttcgatttttgggggcaaggaacattggtgaccgtcagttca

SEQ ID NO: 207 - DNA, 321 bp, synthetic construct
  source 1..321: mol_type = other DNA
gacatccagatgacccagtctccatcctcactgtctgcatctgtgggagacagagtcaccatcacttgtcgggccagtcagggtattagcagctggctggcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatatttatccctacaccttcggacaaggtacaaaactggagatcaaa

SEQ ID NO: 208 - protein, 218 aa, synthetic construct
DIQMTQSPSSLSASVGDRVTITCRASQSVDEEGDSYMNWYQQKPGKAPKLLIYAASNLESGVPSRFSGSGSGTDFTLTISSLQPEDFATYYCQQSNEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 448 aa, synthetic construct
EVQLVESGGGLVQPGGSLRLSCAASGFTFSSYAMSWVRQAPGKGLEWVASISSGDTTYYPDSVKGRFTISRDNAKNSLYLQMNSLRAEDTAVYYCARNEGYYFGLTLWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 210 - protein, 122 aa, synthetic construct
QMQLVESGGGVVQPGRSLRLSCAASGFTFRTYGMHWVRQAPGKGLEWVAVIWYDGSNKHYADSVKGRFTISRDNSKNTLYLQMNSLRAEDTAVYYCARAPQWELVHEAFDIWGQGTMVTVSS

SEQ ID NO: 211 - protein, 11 aa, synthetic construct
QVWDSSSKHVV

SEQ ID NO: 212 - protein, 11 aa, synthetic construct
QVWDKSSDHVV

SEQ ID NO: 213 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSKHVVFGGGTKLTVL

SEQ ID NO: 214 - protein, 108 aa, synthetic construct
SYVLTQPPSVSVAPGQTARITCGGNNLGSKSVHWYQQKPGQAPVLVVYDDKDRPSWIPERFSGSNSGNTATLTISRGEAGDEADYYCQVWDKSSDHVVFGGGTKLTVL

SEQ ID NO: 215 - protein, 214 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSKHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 216 - protein, 214 aa, synthetic construct
SYVLTQPPSVSVAPGQTARITCGGNNLGSKSVHWYQQKPGQAPVLVVYDDKDRPSWIPERFSGSNSGNTATLTISRGEAGDEADYYCQVWDKSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 217 - DNA, 324 bp, synthetic construct
  source 1..324: mol_type = other DNA
tcctatgtgctgactcagccaccctccgtgtcagtggccccaggacagacagccaggattacctgtgggggaaacaaccttggaagtaaaagtgtgcactggtaccagcagaagccaggccaggcccctgtgctggtcgtctatgatgatagcgaccggccctcatggattcctgagcgattctctggctccaactctgggaacaccgccaccctgaccatcagcaggggcgaagccggggatgaggccgactattactgtcaggtgtgggatagtagtagtaagcatgtggtattcggcggagggaccaagctgaccgtccta

SEQ ID NO: 218 - DNA, 324 bp, synthetic construct
  source 1..324: mol_type = other DNA
tcctatgtgctgactcagccaccctccgtgtcagtggccccaggacagacagccaggattacctgtgggggaaacaaccttggaagtaaaagtgtgcactggtaccagcagaagccaggccaggcccctgtgctggtcgtctatgatgataaggaccggccctcatggattcctgagcgattctctggctccaactctgggaacaccgccaccctgaccatcagcaggggcgaagccggggatgaggccgactattactgtcaggtgtgggataagagtagtgatcatgtggtattcggcggagggaccaagctgaccgtccta

SEQ ID NO: 219 - DNA, 642 bp, synthetic construct
  source 1..642: mol_type = other DNA
tcctatgtgctgactcagccaccctccgtgtcagtggccccaggacagacagccaggattacctgtgggggaaacaaccttggaagtaaaagtgtgcactggtaccagcagaagccaggccaggcccctgtgctggtcgtctatgatgatagcgaccggccctcatggattcctgagcgattctctggctccaactctgggaacaccgccaccctgaccatcagcaggggcgaagccggggatgaggccgactattactgtcaggtgtgggatagtagtagtaagcatgtggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 220 - DNA, 642 bp, synthetic construct
  source 1..642: mol_type = other DNA
tcctatgtgctgactcagccaccctccgtgtcagtggccccaggacagacagccaggattacctgtgggggaaacaaccttggaagtaaaagtgtgcactggtaccagcagaagccaggccaggcccctgtgctggtcgtctatgatgataaggaccggccctcatggattcctgagcgattctctggctccaactctgggaacaccgccaccctgaccatcagcaggggcgaagccggggatgaggccgactattactgtcaggtgtgggataagagtagtgatcatgtggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 221 - protein, 122 aa, synthetic construct
EMQLVESGGGLVQPGGSLRLSCAASGFTFRTYGMHWVRQAPGKGLEWVAVIWYDGSNKHYADSVKGRFTISRDNAKNSLYLQMNSLRAEDTAVYYCARAPQWYLVHEAFDIWGQGTMVTVSS

SEQ ID NO: 222 - protein, 451 aa, synthetic construct
EMQLVESGGGLVQPGGSLRLSCAASGFTFRTYGMHWVRQAPGKGLEWVAVIWYDGSNKHYADSVKGRFTISRDNAKNSLYLQMNSLRAEDTAVYYCARAPQWYLVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 223 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDKSSDHVVFGGGTKLTVL

SEQ ID NO: 224 - protein, 214 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDKSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 225 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 226 - protein, 221 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 442 aa, synthetic construct
EVQLVQSGGGVVQPGRSLRLSCAASGFTFSSYWMSWVRQAPGKGLEWVSAISGSGGSTYYADSVKGRFTISRDNSKNTLYLQMNSLRAEDTAVYYCAKENWGSYFDLWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 228 - protein, 214 aa, synthetic construct
DIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2024/0117030 A1
(43) Pub. Date: Apr. 11, 2024

(54) MULTISPECIFIC ANTIBODIES AND USES THEREOF

(71) Applicant: Pfizer Inc., New York, NY (US)

(72) Inventors: Rita Diane AGOSTINELLI, Lowell, MA (US); James Reasoner APGAR, Newton, MA (US); Eric Matthew BENNETT, Arlington, MA (US); Laird BLOOM, Needham, MA (US); Ting CHEN, Acton, MA (US); Aaron Michael D'ANTONA, Melrose, MA (US); Arnab DE, Haverhil, MA (US); Fang JIN, Waban, MA (US); Marion Teresa KASAIAN, Cambridge, MA (US); Matthew Allister LAMBERT, Dublin (IE); Kimberly Ann MARQUETTE, Somerville, MA (US); Virginie MCMANUS, Dublin (IE); Jessica Haewon MIN DEBARTOLO, Needham, MA (US); Nicole Melissa PICHE-NICHOLAS, Waltham, MA (US); Richard Thomas SHELDON, Putman, CT (US); Lioudmila TCHISTIAKOVA, Stoneham, MA (US); Alexander Michael Shuford BARRON, Boston, MA (US); Richard Lee GIESECK, III, Dedham, MA (US); Xiaotian ZHONG, Wayland, MA (US)

(21) Appl. No.: 18/175,796

(22) Filed: Feb. 28, 2023

Related U.S. Application Data
(60) Provisional application No. 63/483,162, filed on Feb. 3, 2023.
(60) Provisional application No. 63/268,817, filed on Mar. 3, 2022.

(51) Int. Cl. C07K 16/24; A61K 45/06; A61P 35/00

(52) U.S. Cl. CPC C07K 16/247; A61K 45/06; A61P 35/00; A61K 2039/505

(57) ABSTRACT

The present invention relates to antibodies that specifically bind to one or more of IL-4, IL-13, IL-33, TSLP, and p40. The present invention further relates to antibodies that bind to one of IL-4, IL-13, IL-33, or TSLP. The invention further relates to multispecific antibodies that specifically bind to IL-4 and IL-13, and at least one other target. The present invention relates to multispecific antibodies that bind IL-4, IL-13, and one of IL-33, TSLP, or p40. The present invention also pertains to related molecules, e.g. nucleic acids which encode such antibodies or multispecific antibodies, compositions, and related methods, e.g., methods for producing and purifying such antibodies and multispecific antibodies, and their use in diagnostics and therapeutics.

### FIELD

The present invention relates to antibodies that specifically bind to one or more of IL-4, IL-13, IL-33, TSLP, and p40. The present invention further relates to antibodies that bind to one of IL-4, IL-13, IL-33, or TSLP. The invention further relates to multispecific antibodies that specifically bind to IL-4 and IL-13, and at least one other target. The present invention relates to multispecific antibodies that bind IL-4, IL-13, and one of IL-33, TSLP, or p40. The present invention also pertains to related molecules, e.g. nucleic acids which encode such antibodies or multispecific antibodies, compositions, and related methods, e.g., methods for producing and purifying such antibodies and multispecific antibodies, and their use in diagnostics and therapeutics.

### REFERENCE TO SEQUENCE LISTING

“The instant application contains a Sequence Listing which has been submitted electronically in .xml format and is hereby incorporated by reference in its entirety. Said .xml file, created on Feb. 9, 2023, is named PC072799 Sequence Listing.xml and is 252 KB in size.”

### BACKGROUND

The present invention related to antibodies that specifically bind to one or more of IL-4, IL-13, IL-33, TSLP, and p40, and compositions, methods, and uses thereof, including use of antibodies of the disclosure to treat one or more diseases or conditions selected from the group consisting of atopic dermatitis, atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, Crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa including treatment and prevention of one or more symptoms symptom associated with the respective disease or condition.

IL-4 and IL-13 are critical drivers of immune activation, leading to inflammation, edema, fibrosis, and pruritus in atopic disorders (48, 49). IL-4 and IL 13 interact with cells through a common receptor, consisting of IL-4Rα and IL-13Rα1 (type II receptor), expressed on monocytes, fibroblasts, keratinocytes, epithelial cells, smooth muscle cells, and other non-lymphoid cell types (29). IL-4 can also activate cells through IL-4Rα/IL-2Rγ common (type I receptor), expressed on T cells, B-cells, and monocytes. Engagement of IL-4Rα through either receptor activates STAT6 to induce atopy-associated genes (29). Although IL-4 and IL-13 can engage a common receptor and signaling pathway, differences in cytokine availability, localization, and receptor binding affinity result in distinct response profiles (49, 50). Further differentiation can result from the type I receptor (IL-4Rα/γc), which drives Th2 differentiation through IL-4 but not IL-13 (51), and cell-surface “decoy” IL-13Rα2, which mediates neutralization and depletion of IL-13 but not IL-4 (52, 53).

The role of IL-4 and IL-13 in atopic disease is supported by genetic associations, extensive validation in preclinical models, and clinical efficacy of IL-4 and IL-13 neutralization in a range of atopic indications (48). Anti-IL-4Rα Dupixent® (dupilumab; Sanofi/Regeneron) blocks responses to both cytokines, and is approved for treatment of moderate-severe atopic dermatitis (AD), asthma, and chronic rhinosinusitis with nasal polyps, which attests to the activity of IL-4 and IL-13 in these indications (54-56). Anti-IL-13 mAbs lebrikizumab (Lilly) (57) and tralokinumab (Adbry™; Leo Pharma) (58, 59) have also demonstrated efficacy in AD, with more limited activity in asthma (60, 61). The effectiveness of anti-IL-13 mAbs in AD suggest a primary role for IL-13 neutralization in efficacy of Dupixent®. Nevertheless, available meta analyses (62, 63) suggest that Dupixent® has superior activity over lebrikizumab and tralokinumab, consistent with an added benefit of IL-4 blockade.

IL-33 is passively released during cell necrosis or when tissues are damaged, suggesting that it may function as an alarmin that alerts the immune system after endothelial or epithelial cell damage during infection, physical stress, or trauma. IL-33 plays important roles in type-2 innate immunity via activation of allergic inflammation-related eosinophils, basophils, mast cells, macrophages, and group 2 innate lymphoid cells (ILC2s) through its receptor ST2 (96).

Thymic stromal lymphoprotein (TSLP) is an epithelial cytokine critical in the initiation and persistence of inflammation. Tezspire® (Tezepelumab, Amgen) is a TSLP antibody approved for the treatment of asthma.

IL-4 and IL-13 are linked primarily to type 2 effector responses. In contrast, IL-12 and IL-23 are implicated in type 1 and type 3 (Th17) responses, respectively (77). IL-12 drives T helper 1 (Th1) cell differentiation and interferon-γ (IFN-γ) production, whereas IL-23 promotes the maintenance of Th17 cells that produce IL-17 and other type 3 cytokines. Type 1 and type 3 responses have been implicated in a range of human inflammatory and autoimmune diseases. A causal role for IL-12p40-containing cytokines has been established through numerous drug approvals (75) (IL-12p40 is hereinafter referred to simply as p40). The p40 neutralizing agent Stelara® (ustekinumab; Janssen) neutralizes both IL-12 and IL-23, and is approved for the treatment of plaque psoriasis, psoriatic arthritis, Crohn's disease, and ulcerative colitis. The IL-23-selective anti-IL-23p19 blocking agents Tremfya® (guselkumab; Janssen), Skyrizi® (risankizumab; Boehringer Ingelheim/AbbVie), and Ilumya® (tildrakizumab; Sun Pharmaceutical) are approved for a range of psoriatic disorders.

Despite the effectiveness of dupilumab, tezepelumab, guselkumab, risankizumab and tildrakizumab, an unmet need remains for safe and effective therapeutics for numerous diseases characterized by inflammatory responses, that address a broad range of pathogenic mechanisms.

### SUMMARY

Provided herein are antibodies (including antigen-binding fragments thereof) that specifically bind to one or more of IL-4, IL-13, IL-33, TSLP, and p40, as well as monomeric and multimeric antibodies thereof, related nucleic acids, uses, and associated methods thereof. The disclosure also provides processes for making, preparing, and producing antibodies that bind to one or more of IL-4, IL-13, IL-33, TSLP, and p40. Antibodies of the disclosure are useful in one or more of diagnosis, prophylaxis, or treatment of disorders or conditions mediated by, or associated with one or more of IL-4, IL-13, IL-33, TSLP, and p40 activity, including, but not limited to atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa. The disclosure further encompasses expression of antibodies, and preparation and manufacture of compositions comprising antibodies of the disclosure, such as medicaments for the use of the antibodies.

Polynucleotides encoding antibodies that bind one or more of IL-4, IL-13, IL-33, TSLP, and p40 are also provided. Polynucleotides encoding antibody heavy chains or light chains, or both are also provided. Host cells that express the antibodies are provided. Methods of treatment using the antibodies are provided. Such methods include, but are not limited to, one or more of methods of treating or methods of preventing diseases associated with or mediated by one or more of IL-4, IL-13, IL-33, TSLP, and p40 expression and or one or more of IL-4, IL-13, IL-33, TSLP, and p40 binding atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, Crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa.

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting. Those skilled in the art will recognize or be able to ascertain using no more than routine experimentation, many equivalents to the specific embodiments of the invention described herein. Such equivalents are intended to be encompassed by the following embodiments (E).

E1. An isolated antibody that specifically binds to IL-33, comprising a heavy chain variable region (IL33-VH) and a light chain variable region (IL33-VL), comprising
(i) the CDR-H1, CDR-H2, and CDR-H3 sequences of SEQ ID NO: 73, and the CDR-L1, CDR-L2, and CDR-L3 sequences of SEQ ID NO: 78;
(ii) the CDR-H1, CDR-H2, and CDR-H3 sequences of SEQ ID NO: 63, and the CDR-L1, CDR-L2, and CDR-L3 sequences of SEQ ID NO: 71; or
(iii) the CDR-H1, CDR-H2, and CDR-H3 sequences of SEQ ID NO: 80, and the CDR-L1, CDR-L2, and CDR-L3 sequences of SEQ ID NO: 81.

E2. An isolated antibody that specifically binds to IL-33, comprising a heavy chain variable region (IL33-VH) and a light chain variable region (IL33-VL), comprising the CDR-H1, CDR-H2, and CDR-H3 sequences of SEQ ID NO: 73, and the CDR-L1, CDR-L2, and CDR-L3 sequences of SEQ ID NO: 78.

E3. An isolated antibody that specifically binds to IL-33, comprising a heavy chain variable region (IL33-VH) and a light chain variable region (IL33-VL), wherein the CDR-H1 comprises the amino acid sequence of SEQ ID NO: 60; the CDR-H2 comprises the amino acid sequence of SEQ ID NO: 61; the CDR-H3 comprises the amino acid sequence of SEQ ID NO: 72; the CDR-L1 comprises the amino acid sequence of SEQ ID NO: 75; the CDR-L2 comprises the amino acid sequence of SEQ ID NO: 76, and the CDR-L3 comprises the amino acid sequence of SEQ ID NO: 77.

E4. The antibody of any one of E1-E3, comprising an IL33-VH framework sequence derived from a human germline VH sequence selected from the group consisting of DP47, DP48, DP50, DP51, DP54, and DP77.

E5. The antibody of any one of E1-E4, comprising an IL33-VH framework sequence derived from a human DP54 germline sequence.

E6. The antibody of any one of E1-E5, comprising an IL33-VL framework sequence derived from a human germline VL sequence selected from the group consisting of DPK1, DPK3, DPK4, DPK5, DPK7, DPK8, and DPK9.

E7. The antibody of any one of E1-E6, comprising an IL33-VL framework sequence derived from a human germline DPK9 sequence.

E8. The antibody of any one of E1-E7, comprising an IL33-VL framework sequence and an IL33-VH framework sequence 98%, 99%, or 100% sequence, and wherein one or both of the IL33-VL framework sequence and the IL33-VH framework sequence is at least 66%, 76%, 80%, 90%, 91%, 92%, 93%, 94%, 95%, 96%, 97%, identical to the human germline sequence from which it was derived.

E9. The antibody of any one of E1-E8, comprising an IL33-VL framework sequence and an IL33-VH framework sequence, and wherein one or both of the IL33-VL framework sequence or the IL33-VH framework sequence is identical to the human germline sequence from which it was derived.

E10. The antibody of any one of E1-E9, comprising an IL33-VH sequence at least 90%, 91%, 92%, 93%, 94%, 95%, 96%, 97%, 98%, 99%, or 100% identical to SEQ ID NO: 73.

E11. The antibody or any one of E1-E10, comprising an IL33-VL sequence at least 90%, 91%, 92%, 93%, 94%, 95%, 96%, 97%, 98%, 99%, or 100% identical to SEQ ID NO: 78.

E12. The antibody of any one of E1-E11, comprising
(i) the IL33-VH sequence of SEQ ID NO: 73, and the IL33-VL of SEQ ID NO: 78; or
(ii) the IL33-VH sequence of SEQ ID NO: 63, and the IL33-VL of SEQ ID NO: 71; or
(iii) the IL33-VH sequence of SEQ ID NO: 80, and the IL33-VL of SEQ ID NO: 81. E13. The antibody of any one of E1-E12, comprising an IL33-VH sequence identical to SEQ ID NO: 73, and an IL33-VL identical to SEQ ID NO: 78.

E14. The antibody of any one of E1-E13, comprising a IL33-VH sequence encoded by a nucleic acid sequence of SEQ ID NO: 202.

E15. The antibody of any one of E1-E14, comprising a IL33-VL sequence encoded by a nucleic acid sequence of SEQ ID NO 203.

E16. The antibody of any one of E1-E15, comprising an IL33-VH sequence encoded by the plasmid deposited at the ATCC and having ATCC Accession No. PTA-127210.

E17. The antibody of any one of E1-E16, comprising an IL33-VL sequence encoded by the plasmid deposited at the ATCC and having ATCC Accession No. PTA-12720

E18. An antibody comprising an IL33-VH sequence encoded by the plasmid deposited at the ATCC and having ATCC Accession No. PTA-127210 and an IL33-VL sequence encoded by the plasmid deposited at the ATCC and having ATCC Accession No. PTA-127209.

E19. The antibody of E1-E18, wherein the antibody binds human IL-33 with a K_{D} less than a value selected from the group consisting of 100 pM, 50 pM, 40 pM, 30 pM, 25 pM, 20 pM, 15 pM, and 10 pM, 5 pM, 2 pM, 1 pM, 750 fM, 500 fM, and 250 fM.

E20. The antibody of E1-E19, wherein the antibody binds human IL-33 with a K_{D} about or less than a value of 15 pM.

E21. The antibody of E1-E20, wherein the antibody binds human IL-33 with a K_{D} about or less than a value of 1 pM.

E22. The antibody of E1-E21, wherein the antibody binds human IL-33 with a K_{D} about or less than a value of 250 fM.

E23. The antibody of any one of E19-E22, wherein the K_{D} value is measured by kinetic exclusion assay.

E24. The antibody of any one of E19-E22, wherein the K_{D} value is measured by surface plasmon resonance (SPR).

E25. The antibody of E1-E24, wherein the IL-33 IC₅₀ is less than 20 pM in a HEK Blue® IL-33 neutralization SEAP assay.

E26. The antibody of E1-E25, wherein the IL-33 IC₅₀ is less than 15 pM in a HEK Blue® IL-33 neutralization SEAP assay.

E27. The antibody of E25-E26, wherein the HEK Blue® IL-33 neutralization SEAP assay is conducted for 20 hours at 37° C.

E28. The antibody of E1-E27, wherein the IL-33 IC₅₀ is less than 15 pM, and is calculated by ELISA measurement of IFNγ in a human whole blood assay treated with IL-33 and IL-12.

E29. The antibody of E28, wherein the human whole blood assay is conducted at 37° C. for 22 hours.

E30. The antibody of E1-E29, wherein the antibody binds cynomolgus IL-33.

E31. The antibody of E1-E30, wherein the binding K_{D} of the antibody to cynomolgus IL-33 is within 3 orders of magnitude of the binding K_{D} of the antibody to human IL-33.

E32. The antibody of E1-E31, further comprising a constant heavy domain (IL33-CH1) and a constant light domain (IL33-CL).

E33. The antibody of E32, wherein the IL33-CH1 comprises a sequence selected from the group consisting of SEQ ID NO: 6, SEQ ID NO: 105, and SEQ ID NO: 110.

E34. The antibody of E32-E33, wherein the IL33-CH1 comprises a sequence according to SEQ ID NO: 6.

E35. The antibody of E32-E34, wherein the IL33-CL comprises a sequence selected from the group consisting of SEQ ID NO: 16, SEQ ID NO: 108, and SEQ ID NO: 113.

E36. The antibody of E32-E35, wherein the IL33-CL comprises a sequence according to SEQ ID NO:16.

E37. The antibody of E32-E36, wherein the IL33-CH1 is connected to the IL33-VL, and the IL33-CL is connected to the IL33-VH forming an IL-33-binding domain-swap Fab domain (IL33-xFab).

E38. The antibody of E32-E37, wherein the IL33-CH1 is connected to the IL33-VH, and the IL33-CL is connected to the IL33-VL forming an IL-33 binding Fab domain (IL33-Fab). E39. The antibody of any one of E1-E38, comprising an antibody Fc domain comprising a first Fc chain and a second Fc chain.

E40. The antibody of E39, wherein the Fc domain is the Fc domain of an IgA (for example IgA₁ or IgA₂), IgD, IgE, IgM, or IgG (for example IgG₁, IgG₂, IgG₃, or IgG₄).

E41. The antibody of E40, wherein the Fc domain is the Fc domain of an IgG₁.

E42. The antibody of E40, wherein the N-terminus of the first Fc chain or the second Fc chain is connected to the C-terminus of the IL33-CH1 domain.

E43. The antibody of E41-E42, wherein the first and second Fc chain each comprises, from N-terminus to C-terminus: a hinge region, a CH2 region, and a CH3 region.

E44. The antibody of E43, wherein the hinge region comprises a sequence selected from the group consisting of SEQ ID NO: 7, SEQ ID NO: 102, SEQ ID NO: 123, SEQ ID NO: 126, SEQ ID NO: 129, and SEQ ID NO: 131.

E45. The antibody of E44, wherein the hinge region comprises a sequence according to SEQ ID NO: 7.

E46. The antibody of E44, wherein the hinge region on the first Fc chain and the hinge region on the second Fc chain comprise a pair of sequences according to SEQ ID NO: 129 and SEQ ID NO: 131.

E47. The antibody of E43-E46, wherein the CH2 region comprises a sequence according to SEQ ID NO: 8.

E48. The antibody of E43-E47, wherein the CH3 region on the first Fc chain and the CH3 region on the second Fc chain comprise a pair of sequences selected from the group consisting of
(i) SEQ ID NO: 9 and SEQ ID NO: 9;
(ii) SEQ ID NO: 111 and SEQ ID NO: 106;
(iii) SEQ ID NO: 111 and SEQ ID NO: 114;
(iv) SEQ ID NO: 114 and SEQ ID NO: 117;
(v) SEQ ID NO: 124 and SEQ ID NO: 127;
(vi) SEQ ID NO: 139 and SEQ ID NO: 141; and
(vii) SEQ ID NO: 147 and SEQ ID NO: 148.

E49. The antibody of E48, wherein the CH3 region on the first Fc chain and the CH region on the second Fc chain each comprise a sequence according to SEQ ID NO: 9.

E50. The antibody of E48, wherein the CH3 region on the first Fc chain and the CH3 region on the second Fc chain comprise a pair of sequences according to SEQ ID NO: 124 and SEQ ID NO: 127.

E51. The antibody of any one of E1-E50, comprising an IL33-VH bearing polypeptide comprising an amino acid sequence at least 90%, 91%, 92%, 93%, 94%, 95%, 96%, 97%, 98%, 98%, 99%, or 100% identical to a sequence selected from the group consisting of SEQ ID NO: 74, SEQ ID NO:103, SEQ ID NO: 128, SEQ ID NO: 132, SEQ ID NO: 134, SEQ ID NO: 137, SEQ ID NO: 142, and SEQ ID NO: 143.

E52. The antibody of any one of E1-E51, comprising an IL33-VH bearing polypeptide comprising an amino acid sequence selected from the group consisting of SEQ ID NO: 74, SEQ ID NO:103, SEQ ID NO: 128, SEQ ID NO: 132, SEQ ID NO: 134, SEQ ID NO: 137, SEQ ID NO: 142, and SEQ ID NO: 143.

E53. The antibody of any one of E52-E53, wherein the IL33-VH bearing polypeptide comprises a sequence according to SEQ ID NO: 74.

E54. The antibody of any one of E52-E52, wherein the IL33-VH bearing polypeptide comprises a sequence according to SEQ ID NO: 132.

E55. The antibody of E32-E54, wherein the IL33-CL comprises an amino acid sequence selected from the group consisting of SEQ ID NO: 16, SEQ ID NO: 108, SEQ ID NO: 113.

E56. The antibody of E32-E55, wherein the IL33-CL comprises an amino acid sequence of SEQ ID NO:16.

E57. The antibody of any one of E1-56, comprising an IL33-VL bearing polypeptide comprising an amino acid sequence at least 90%, 91%, 92%, 93%, 94%, 95%, 96%, 97%, 98%, 99%, or 100% identical to a sequence selected from the group consisting of SEQ ID NO: 79, SEQ ID NO: 107, SEQ ID NO: 115, SEQ ID NO: 121, and SEQ ID NO: 138, SEQ ID NO: 144, and SEQ ID NO: 145.

E58. The antibody of any one of E1-E57, comprising an IL33-VL bearing polypeptide comprising the amino acid sequence of SEQ ID NO: 79.

E59. The antibody of E1-E58, comprising an IL33-VH bearing polypeptide of SEQ ID NO: 74 and an IL33-VL bearing polypeptide of SEQ ID NO: 79.

E60. The antibody of E1-E59, comprising an IL33-VH bearing polypeptide of SEQ ID NO: 132 and an IL33-VL bearing polypeptide of SEQ ID NO: 79.

E61. The antibody of any one of E1-E60, comprising an IL33-VH bearing polypeptide sequence encoded by the plasmid deposited at the ATCC and having ATCC Accession No. PTA-127208.

E62. The antibody of any one of E1-E61, comprising an IL33-VL bearing polypeptide sequence encoded by the plasmid deposited at the ATCC and having ATCC Accession No. PTA-127207.

E63. An antibody comprising an IL33-VH bearing polypeptide sequence encoded by the plasmid deposited at the ATCC and having ATCC Accession No. PTA-127208, and an IL33-VL bearing polypeptide encoded by the plasmid deposited at the ATCC and having ATCC Accession No. PTA-127207.

E64. An isolated antibody that specifically binds IL-33, comprising the CDRs of an antibody selected from one or more of Tables 82, 85, and 87.

E65. An isolated antibody that specifically binds IL-33, comprising the VH and VL of an antibody selected from one or more of Tables 82, 84, and 87.

E66. An isolated antibody that specifically binds IL-33, selected from one or more of Tables 82, 84, and 87.

E67. The antibody of any one of E1-65, for use as a medicament.

E68. The antibody of E67, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, diabetic kidney disease, Behcet's disease, gout, Alzheimer's disease, and atherosclerosis

E69. The antibody of any one of E67-E68, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, and non-alcoholic steatohepatitis (NASH).

E70. The antibody of any one of E67-E69, wherein the use is for atopic dermatitis.

E71. The antibody of any one of E67-E69, wherein the use is for non-alcoholic steatohepatitis (NASH).

E72. A pharmaceutical composition comprising a therapeutically effective amount of the antibody of E1-E71 and a pharmaceutically acceptable carrier.

E73. A method of treating a medical condition, comprising administering to a subject in need thereof a therapeutically effective amount of the antibody of any one of E1-E71, or the pharmaceutical composition of E72.

E74. The method of E73, wherein the condition is selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, diabetic kidney disease, Behcet's disease, gout, Alzheimer's disease, and atherosclerosis.

E75. The method of any one of E73-E74, comprising administering said antibody or pharmaceutical composition, subcutaneously.

E76. The method of any one of E73-E75, wherein said antibody thereof, or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E77. An isolated antibody that specifically binds to TSLP, comprising a heavy chain variable region (TSLP-VH) and a light chain variable region (TSLP-VL), comprising
(i) the CDR-H1, CDR-H2, and CDR-H3 sequences of SEQ ID NO: 92, and the CDR-L1, CDR-L2, and CDR-L3 sequences of SEQ ID NO: 94;
(ii) the CDR-H1, CDR-H2, and CDR-H3 sequences of SEQ ID NO: 92, and the CDR-L1, CDR-L2, and CDR-L3 sequences of SEQ ID NO: 93;
(iii) the CDR-H1, CDR-H2, and CDR-H3 sequences of SEQ ID NO: 92, and the CDR-L1, CDR-L2, and CDR-L3 sequences of SEQ ID NO: 213; or
(iv) the CDR-H1, CDR-H2, and CDR-H3 sequences of SEQ ID NO: 92, and the CDR-11, CDR-L2, and CDR-L3 sequences of SEQ ID NO: 214;
(v) the CDR-H1, CDR-H2, and CDR-H3 sequences of SEQ ID NO: 221, and the CDR-11, CDR-L2, and CDR-L3 sequences of SEQ ID NO: 213;
(vi) the CDR-H1, CDR-H2, and CDR-H3 sequences of SEQ ID NO: 221, and the CDR-11, CDR-L2, and CDR-L3 sequences of SEQ ID NO: 94; or
(vii) the CDR-H1, CDR-H2, and CDR-H3 sequences of SEQ ID NO: 221, and the CDR-11, CDR-L2, and CDR-L3 sequences of SEQ ID NO: 223.

E78. An isolated antibody that specifically binds to TSLP, comprising a heavy chain variable region (TSLP-VH) and a light chain variable region (TSLP-VL), comprising the CDR-H1, CDR-H2, and CDR-H3 sequences of SEQ ID NO: 92, and the CDR-L1, CDR-L2, and CDR-L3 sequences of SEQ ID NO: 94.

E79. An isolated antibody that specifically binds to TSLP, comprising
(i) a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 90;
(ii) a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 211;
(iii) a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 212;
(iv) a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 211;
(v) a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 90; or
(vi) a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 212.

E80. The antibody of any one of E77-E79, comprising a TSLP-VH framework sequence derived from a human germline VH sequence selected from the group consisting of DP47, DP49, DP50, DP54, and DP53.

E81. The antibody of any one of E77-E80, comprising a TSLP-VH framework sequence derived from a human DP50 germline sequence.

E82. The antibody of any one of E77-E81, comprising a TSLP-VL framework sequence derived from a human germline VL sequence selected from the group consisting of DPL16, DPL23, V2-6, V2-8, V2-14, and V2-17.

E83. The antibody of any one of E77-E82, comprising a TSLP-VL framework sequence derived from a human germline V2-14 sequence

E84. The antibody of any one of E77-E84, comprising a TSLP-VL framework sequence and a TSLP-VH framework sequence, and wherein one or both of the TSLP-VL framework sequence or TSLP-VH framework sequence is at least 66%, 76%, 80%, 90%, 91%, 92%, 93%, 94%, 95%, 96%, 97%, 98%, 99%, or 100% identical to the human germline sequence from which it was derived.

E85. The antibody of any one of E77-E84, comprising a TSLP-VL framework sequence and a TSLP-VH framework sequence, and wherein one or both of the TSLP-VL framework sequence or the TSLP-VH framework sequence is identical to the human germline sequence from which it was derived.

E86. The antibody of any one of E77-E85, comprising a TSLP-VH sequence at least 90%, 91%, 92%, 93%, 94%, 95%, 96%, 97%, 98%, 99%, or 100% identical to SEQ ID NO: 92.

E87. The antibody of any one of E77-E86, comprising a TSLP-VL sequence at least 90%, 91%, 92%, 93%, 94%, 95%, 96%, 97%, 98%, 99%, or 100% identical to SEQ ID NO: 94.

E88. The antibody of any one of E77-E86, comprising
(i) the TSLP-VH sequence of SEQ ID NO: 92, and the TSLP-VL of SEQ ID NO: 94;
(ii) the TSLP-VH sequence of SEQ ID NO: 92, and the TSLP-VL of SEQ ID NO: 93;
(iii) the TSLP-VH sequence of SEQ ID NO: 92, and the TSLP-VL of SEQ ID NO: 213;
(iv) the TSLP-VH sequence of SEQ ID NO: 92, and the TSLP-VL of SEQ ID NO: 214;
(v) the TSLP-VH sequence of SEQ ID NO: 221, and the TSLP-VL of SEQ ID NO: 215;
(vi) the TSLP-VH sequence of SEQ ID NO: 221, and the TSLP-VL of SEQ ID NO: 99; or
(vii) the TSLP-VH sequence of SEQ ID NO: 221, and the TSLP-VL of SEQ ID NO: 224;

E89. The antibody of any one of E77-E88, comprising a TSLP-VH sequence identical to SEQ ID NO: 92, and a TSLP-VL identical to SEQ ID NO: 94.

E90. The antibody of any one of E77-E88, comprising a TSLP-VH sequence identical to SEQ ID NO: 92, and a TSLP-VL identical to SEQ ID NO: 93.

E91. The antibody of any one of E77-E88, comprising a TSLP-VH sequence identical to SEQ ID NO: 92, and a TSLP-VL identical to SEQ ID NO: 213.

E92. The antibody of any one of E77-E88, comprising a TSLP-VH sequence identical to SEQ ID NO: 92, and a TSLP-VL identical to SEQ ID NO: 214.

E93. The antibody of any one of E77-E89, comprising the TSLP-VL sequence encoded by a nucleic acid sequence of SEQ ID NO: 205.

E94. The antibody of E91, comprising the TSLP-VL sequence encoded by a nucleic acid sequence of SEQ ID NO: 217.

E95. The antibody of any one of E92, comprising the TSLP-VL sequence encoded by a nucleic acid sequence of SEQ ID NO: 218.

E96. The antibody of any one of E77-E95, comprising the TSLP-VH sequence encoded by a nucleic acid sequence of SEQ ID NO: 204.

E97. The antibody of any one of E77-E96, comprising the TSLP-VH sequence encoded by the plasmid deposited at the ATCC and having ATCC Accession No. PTA-127200.

E98. The antibody of any one of E77-E89, comprising the TSLP-VL sequence encoded by the plasmid deposited at the ATCC and having ATCC Accession No. PTA-127199.

E99. An antibody comprising the TSLP-VH sequence encoded by the plasmid deposited at the ATCC and having ATCC Accession No. PTA-127200, and the comprising the TSLP-VL sequence encoded by the plasmid deposited at the ATCC and having ATCC Accession No. PTA-127199.

E100. The antibody of any one of E77-E88, comprising the TSLP-VL sequence encoded by the plasmid deposited at the ATCC and having ATCC Accession No. PTA-______.

E101. An antibody comprising the TSLP-VH sequence encoded by the plasmid deposited at the ATCC and having ATCC Accession No. PTA-127200, and the comprising the TSLP-VL sequence encoded by the plasmid deposited at the ATCC and having ATCC Accession No. PTA-______.

E102. The antibody of any one of E77-E88, comprising the TSLP-VL sequence encoded by the plasmid deposited at the ATCC and having ATCC Accession No. PTA-______.

E103. An antibody comprising the TSLP-VH sequence encoded by the plasmid deposited at the ATCC and having ATCC Accession No. PTA-127200, and the comprising the TSLP-VL sequence encoded by the plasmid deposited at the ATCC and having ATCC Accession No. PTA-______.

E104. The antibody of E77-E102, wherein the antibody is characterized by an IC₅₀ of less than 10 pM in a TARC production bioassay in human peripheral blood monocytes.

E105. The antibody of E77-E104, wherein the antibody is characterized by an IC₅₀ of less than 7 pM in a TARC production bioassay in human peripheral blood monocytes.

E106. The antibody of E77-E105, wherein the antibody is characterized by an IC₅₀ of less than 6 pM in a TARC production bioassay in human peripheral blood monocytes.

E107. The antibody of E77-E106, wherein the antibody has a melting temperature of 68° C.

E108 The antibody of E77-E107, wherein the pH3.4 hold Δ% HMMS is less than 5, such that the pH3.4 hold Δ% HMMS is defined as the difference between the percentage of high molecular weight species due to degradation after 5 hours of incubation of the antibody at room temperature at pH3.4 and the percentage of high molecular weight species due to degradation after 5 hours of incubation of the antibody at room temperature at pH 7.2.

E109. The antibody of E77-E108, wherein the antibody has a low pH3.4 hold Δ% HMMS of less than 1.

E110. The antibody of E77-E109, wherein the antibody has a low pH3.4 hold Δ% HMMS of less than 0.1.

E111. The antibody of E77-E110, further comprising a constant heavy domain (TSLP-CH1) and a constant light domain (TSLP-CL).

E112. The antibody of E111, wherein the TSLP-CH1 comprises a sequence selected from the group consisting of SEQ ID NO: 6, SEQ ID NO: 105, and SEQ ID NO: 110.

E113. The antibody of E111-E112, wherein the TSLP-CH1 comprises a sequence according to SEQ ID NO: 6.

E114. The antibody of E111-E113, wherein the TSLP-CL comprises a sequence selected from the group consisting of SEQ ID NO: 16, SEQ ID NO: 95, SEQ ID NO: 108, and SEQ ID NO: 113.

E115. The antibody of E111-E114, wherein the TSLP-CL comprises a sequence according to SEQ ID NO: 95.

E116. The antibody of E111-E115, wherein the TSLP-CH1 is connected to the TSLP-VL, and the TSLP-CL is connected to the TSLP-VH forming a TSLP-binding domain-swap Fab domain (TSLP-xFab).

E117. The antibody of E111-E115, wherein the TSLP-CH1 is connected to the TSLP-VH, and the TSLP-CL is connected to the TSLP-VL forming a TSLP binding Fab domain (TSLP-Fab).

E118. The antibody of any one of E77-E117, comprising an Fc domain comprising a first Fc chain and a second Fc chain.

E119. The antibody of E118, wherein the Fc domain is the Fc domain of an IgA (for example IgA₁ or IgA₂), IgD, IgE, IgM, or IgG (for example IgG₁, IgG₂, IgG₃, or IgG₄).

E120. The antibody of any one of E118-E119 wherein the Fc domain is the Fc domain of an IgG₁.

E121. The antibody of E118-E120, wherein the N-terminus of the first Fc chain or the second Fc chain is connected to the C-terminus of the TSLP-CH1 domain.

E122. The antibody of E118-E121, wherein the first Fc chain and the second Fc chain each comprises, from N-terminus to C-terminus: a hinge region, a CH2 region, and a CH3 region.

E123. The antibody of E122, wherein the hinge region comprises a sequence selected from the group consisting of SEQ ID NO: 7, SEQ ID NO: 102, SEQ ID NO: 123, SEQ ID NO:126, SEQ ID NO: 129, and SEQ ID NO:131.

E124. The antibody of E122-E123, wherein the hinge region comprises a sequence according to SEQ ID NO: 7.

E125. The antibody of E122-E123, wherein the hinge region on the first Fc chain and the hinge region on the second Fc chain comprise a pair of sequences according to SEQ ID NO: 129 and SEQ ID NO: 131.

E126. The antibody of E122-E123, wherein the CH2 region comprises a sequence according to SEQ ID NO: 8.

E127. The antibody of E122-E126, wherein the CH3 region on the first Fc chain and the CH3 region on the second Fc chain comprise a pair of sequences selected from the group consisting of
(i) SEQ ID NO: 124 and SEQ ID NO: 127;
(ii) SEQ ID NO: 9 and SEQ ID NO: 9;
(iii) SEQ ID NO: 111 and SEQ ID NO: 106;
(iv) SEQ ID NO: 111 and SEQ ID NO: 114;
(v) SEQ ID NO: 114 and SEQ ID NO: 117;
(vi) SEQ ID NO: 139 and SEQ ID NO: 141; and
(vii) SEQ ID NO: 147 and SEQ ID NO: 148.

E128. The antibody of E127, wherein the CH3 region on the first Fc chain and the CH region on the second Fc chain each comprise a sequence according to SEQ ID NO: 9.

E129. The antibody of E128, wherein the CH3 region on the first Fc chain and the CH region on the second Fc chain comprise a pair of sequences according to SEQ ID NO: 124 and SEQ ID NO: 127.

E130. The antibody of any one of E77-E129, comprising a TSLP-VH bearing polypeptide comprising an amino acid sequence at least 90%, 91%, 92%, 93%, 94%, 95%, 96%, 97%, 98%, 99%, or 100% identical to a sequence selected from the group consisting of SEQ ID NO: 97, SEQ ID NO: 152, SEQ ID NO: 153, SEQ ID NO: 155, SEQ ID NO: 158, SEQ ID NO: 161, SEQ ID NO:165, SEQ ID NO: 222

E131. The antibody of any one of E77-E130, comprising a TSLP-VH bearing polypeptide comprising the amino acid sequence selected from the group consisting of SEQ ID NO: 97, SEQ ID NO: 152, SEQ ID NO: 153, SEQ ID NO: 155, SEQ ID NO: 158, SEQ ID NO: 161, SEQ ID NO:165, and SEQ ID NO: 222.

E132. The antibody of any one of E77-E131, wherein the TSLP-VH bearing polypeptide comprises a sequence according to SEQ ID NO: 97.

E133. The antibody of any one of E77-E131, wherein the TSLP-VH bearing polypeptide comprises a sequence according to SEQ ID NO: 165.

E134. The antibody of any one of E77-E133, comprising a TSLP-VL bearing polypeptide comprising an amino acid sequence at least 90%, 91%, 92%, 93%, 94%, 95%, 96%, 97%, 98%, 99%, or 100% identical to a sequence selected from the group consisting of SEQ ID NO: 98, SEQ ID NO: 99, SEQ ID NO: 150, SEQ ID NO: 154, SEQ ID NO: 156, SEQ ID NO: 157, SEQ ID NO: 160, SEQ ID NO: 215, SEQ ID NO: 216, and SEQ ID NO: 224.

E135. The antibody of any one of E77-E134, comprising a TSLP-VL bearing polypeptide comprising a sequence selected from the group consisting of SEQ ID NO: 98, SEQ ID NO: 99, SEQ ID NO: 150, SEQ ID NO: 154, SEQ ID NO: 156, SEQ ID NO: 157, SEQ ID NO: 160, SEQ ID NO: 215, SEQ ID NO: 216, and SEQ ID NO: 224.

E136. The antibody of any one of E77-E135, comprising a TSLP-VL bearing polypeptide comprising a sequence according to SEQ ID NO: 99.

E137. The antibody of any one of E77-E135, comprising the TSLP-VL bearing polypeptide sequence encoded by the plasmid deposited at the ATCC and having ATCC Accession No. PTA-127201.

E138. The antibody of any one of E77-E135, comprising the TSLP-VL bearing polypeptide sequence encoded by the plasmid deposited at the ATCC and having ATCC Accession No. PTA-______.

E139. The antibody of any one of E77-E135, comprising the TSLP-VL bearing polypeptide sequence encoded by the plasmid deposited at the ATCC and having ATCC Accession No. PTA-______.

E140. The antibody of any one of E77-E139, comprising the TSLP-VH bearing polypeptide sequence encoded by the plasmid deposited at the ATCC and having ATCC Accession No. PTA-127202.

E141. An antibody comprising a TSLP-VH bearing polypeptide sequence encoded by the plasmid deposited at the ATCC and having ATCC Accession No. PTA-127202, and a TSLP-VL bearing polypeptide sequence encoded by the plasmid deposited at the ATCC and having ATCC Accession No. PTA-127201.

E142. An antibody comprising a TSLP-VH bearing polypeptide sequence encoded by the plasmid deposited at the ATCC and having ATCC Accession No. PTA-127202, and a TSLP-VL bearing polypeptide sequence encoded by the plasmid deposited at the ATCC and having ATCC Accession No. PTA-______.

E143. An antibody comprising a TSLP-VH bearing polypeptide sequence encoded by the plasmid deposited at the ATCC and having ATCC Accession No. PTA-127202, and a TSLP-VL bearing polypeptide sequence encoded by the plasmid deposited at the ATCC and having ATCC Accession No. PTA-______.

E144. An isolated antibody that specifically binds to p40 through a p40 binding domain and wherein the antibody comprises at least one additional antigen binding domain that specifically binds to an antigen selected from the group consisting of IL-4, IL-13, IL-33, and TSLP, wherein the p40 binding domain comprises a heavy chain variable region (p40-VH) and a light chain variable region (p40-VL), comprising the CDR-H1, CDR-H2, and CDR-H3 sequence of SEQ ID NO: 169, and the CDR-L1, CDR-L2, and CDR-L3 sequences of SEQ ID NO: 175.

E145. An isolated antibody that specifically binds to p40 through a p40 binding domain and wherein the antibody comprises at least one additional antigen binding domain that specifically binds to an antigen selected from the group consisting of IL-4, IL-13, IL-33, and TSLP, and wherein the p40 binding domain comprises a heavy chain variable region (p40-VH) and a light chain variable region (p40-VL), wherein the CDR-H1 of the p40 binding domain comprises the amino acid sequence of SEQ ID NO: 166; the CDR-H2 of the p40 binding domain comprises the amino acid sequence of SEQ ID NO: 167; the CDR-H3 of the p40 binding domain comprises the amino acid sequence of SEQ ID NO: 168; the CDR-L1 of the p40 binding domain comprises the amino acid sequence of SEQ ID NO: 171; the CDR-L2 of the p40 binding domain comprises the amino acid sequence of SEQ ID NO: 172, and the CDR-L3 of the p40 binding domain comprises the amino acid sequence of SEQ ID NO: 173.

E146. The antibody of any one of E144-E145, wherein the antibody further comprises one or both of an IL-4 binding domain that specifically binds to IL-4, and an IL-13 binding domain that specifically binds to IL-13.

E147. The antibody of any one of E144-E146, wherein the p40 binding domain comprises a p40-VH framework sequence derived from a human germline VH sequence selected from the group consisting of DP3, DP7, DP73, DP75, and DP88.

E148. The antibody of any one of E144-E147, wherein the p40 binding domain comprises a p40-VH framework sequence derived from a human DP73 germline sequence.

E149. The antibody of any one of E144-E148, wherein the p40 binding domain comprises a p40-VL framework sequence derived from a human germline VL sequence selected from the group consisting of DPK4, DPK5, DPK7, DPK8, and DPK9.

E150. The antibody of any one of E144-E149, wherein the p40 binding domain comprises a p40-VL framework sequence derived from a human germline DPK7 sequence

E151. The antibody of any one of E144-E150, wherein the p40 binding domain comprises a p40-VL framework sequence and a p40-VH framework sequence, and wherein one or both of the p40 binding domain p40-VL framework sequence and the p40 binding domain p40-VH framework sequence is at least 66%, 76%, 80%, 90%, 91%, 92%, 93%, 94%, 95%, 96%, 97%, 98%, 99%, or 100% identical to the human germline sequence from which it was derived.

E152. The antibody of any one of E144-E151, wherein the p40 binding domain comprises a p40-VL framework sequence and a p40-VH framework sequence, and wherein one or both of the p40-VL framework sequence or the p40-VH framework sequence is identical to the human germline sequence from which it was derived.

E153. The antibody of any one of E144-E152, wherein the p40 binding domain comprises a p40-VH at least 90%, 91%, 92%, 93%, 94%, 95%, 96%, 97%, 98%, 99%, or 100% identical to SEQ ID NO: 169.

E154. The antibody of any one of E144-E153, wherein the p40 binding domain comprises a p40-VL at least 90%, 91%, 92%, 93%, 94%, 95%, 96%, 97%, 98%, 99%, or 100% identical to SEQ ID NO: 175.

E155. The antibody of any one of E144-E154, wherein the p40 binding domain comprises a p40-VH of SEQ ID NO: 169, and a p40-VL of SEQ ID NO: 175.

E156. The antibody of any one of E144-E155, wherein the p40 binding domain comprises a p40-VH sequence encoded by a nucleic acid sequence of SEQ ID NO: 206.

E157. The antibody of any one of E145-E156, wherein the p40 binding domain comprises a p40-VL sequence encoded by a nucleic acid sequence of SEQ ID NO: 207.

E158. The antibody of any one of E144-E157, wherein the p40 binding domain comprises a p40-VH sequence encoded by the plasmid deposited at the ATCC and having ATCC Accession No. PTA-127206.

E159. The antibody of any one of E144-E158, wherein the p40 binding domain comprises a p40-VL sequence encoded by the plasmid deposited at the ATCC and having ATCC Accession No. PTA-127205.

E160. The antibody of E144-E159, further comprising a constant heavy domain (p40-CH1) and a constant light domain (p40-CL).

E161. The antibody of E160, wherein the p40-CH1 comprises a sequence selected from the group consisting of SEQ ID NO: 6, SEQ ID NO: 105, and SEQ ID NO: 110.

E162. The antibody of E160-E161, wherein the p40-CH1 comprises a sequence according to SEQ ID NO: 6.

E163. The antibody of E160-E162, wherein the p40-CL comprises a sequence selected from the group consisting of SEQ ID NO: 16, SEQ ID NO: 108, and SEQ ID NO: 113.

E164. The antibody of E160-E163, wherein the p40-CL comprises a sequence according to SEQ ID NO:16.

E165. The antibody of E160-E164, wherein the p40-CH1 is connected to the p40-VL, and the p40-CL is connected to the p40-VH forming a p40-binding domain-swap Fab domain (p40-xFab).

E166. The antibody of E160-E164, wherein the p40-CH1 is connected to the p40-VH, and the p40-CL is connected to the p40-VL forming a p40 binding Fab domain (p40-Fab).

E167. The antibody of any one of E144-E166, comprising an antibody Fc domain comprising a first Fc chain and a second Fc chain.

E168. The antibody of E167, wherein the Fc domain is the Fc domain of an IgA (for example IgA₁ or IgA₂), IgD, IgE, IgM, or IgG (for example IgG₁, IgG₂, IgG₃, or IgG₄).

E169. The antibody of E168 wherein the Fc domain is the Fc domain of an IgG₁.

E170. The antibody of any one of E167-E169, wherein the N-terminus of the first Fc chain or the second Fc chain is connected to the C-terminus of the p40-CH1 domain.

E171. The antibody of E167-E170, wherein the first and second Fc chain each comprises, from N-terminus to C-terminus: a hinge region, a CH2 region, and a CH3 region.

E172. The antibody of E171, wherein the hinge region comprises a sequence selected from the group consisting of SEQ ID NO: 7, SEQ ID NO: 102, SEQ ID NO: 123, SEQ ID NO:126, SEQ ID NO: 129, and SEQ ID NO:131.

E173. The antibody of E171-E172, wherein the hinge region on the first Fc chain and the hinge region on the second Fc chain comprise a pair of sequences according to SEQ ID NO: 129 and SEQ ID NO: 131.

E174. The antibody of E171-E172, wherein the CH2 region comprises a sequence according to SEQ ID NO: 8.

E175. The antibody of E171-E174, wherein the CH3 region on the first Fc chain and the CH3 region on the second Fc chain comprise a pair of sequences selected from the group consisting of
(i) SEQ ID NO: 9 and SEQ ID NO: 9;
(ii) SEQ ID NO: 111 and SEQ ID NO: 106;
(iii) SEQ ID NO: 111 and SEQ ID NO: 114;
(iv) SEQ ID NO: 114 and SEQ ID NO: 117;
(v) SEQ ID NO: 124 and SEQ ID NO: 127;
(vi) SEQ ID NO: 139 and SEQ ID NO: 141; and
(vii) SEQ ID NO: 147 and SEQ ID NO: 148.

E176. The antibody of E171-E175, wherein the CH3 region on the first Fc chain and the CH region on the second Fc chain each comprise a sequence according to SEQ ID NO: 9.

E177. The antibody of E171-E176, wherein the CH3 region on the first Fc chain and the CH region on the second Fc chain comprise a pair of sequences according to SEQ ID NO: 124 and SEQ ID NO: 127.

E178. The antibody of any one of E144-E177, comprising a p40-VH bearing polypeptide comprising an amino acid sequence at least 90%, 91%, 92%, 93%, 94%, 95%, 96%, 97%, 98%, 99%, or 100% identical to a sequence selected from the group consisting of SEQ ID NO: 170, SEQ ID NO: 177, SEQ ID NO: 181, SEQ ID NO: 185, and SEQ ID NO: 186.

E179. The antibody of any one of E144-E178, comprising a p40-VH bearing polypeptide comprising an amino acid selected from the group consisting of SEQ ID NO: 170, SEQ ID NO: 177, SEQ ID NO: 181, SEQ ID NO: 185, and SEQ ID NO: 186.

E180. The antibody of any one of E144-E179, comprising a p40-VH bearing polypeptide comprising the amino acid sequence of SEQ ID NO: 186.

E181. The antibody of any one of E144-E180, comprising a p40-VL bearing polypeptide comprising an amino acid sequence at least 90%, 91%, 92%, 93%, 94%, 95%, 96%, 97%, 98%, 99%, or 100% identical to a sequence selected from the group consisting of SEQ ID NO: 176, SEQ ID NO: 178, SEQ ID NO: 179, SEQ ID NO: 182, and SEQ ID NO: 183.

E182. The antibody of any one of E144-E181, comprising a p40-VL bearing polypeptide comprising the amino acid sequence of SEQ ID NO: 176.

E183. The antibody of any one of E144-E182, comprising a p40-VH bearing polypeptide sequence encoded by the plasmid deposited at the ATCC and having ATCC Accession No. PTA-127204.

E184. The antibody of any one of E144-E183, comprising a p40-VL bearing polypeptide sequence encoded by the plasmid deposited at the ATCC and having ATCC Accession No. PTA-127203.

E185. An antibody comprising a p40-VH bearing polypeptide sequence encoded by the plasmid deposited at the ATCC and having ATCC Accession No. PTA-127204, and a p40-VL bearing polypeptide sequence encoded by the plasmid deposited at the ATCC and having ATCC Accession No. PTA-127203.

E186. An isolated antibody that binds to p40 and one or both of IL-4, IL-13, comprising the CDRs of an antibody selected from one or more of Tables 86 and 87.

E187. An isolated antibody that binds to p40 and one or both of IL-4, IL-13, comprising the VH and VL of an antibody selected from one or more of Tables 86 and 87.

E188. An isolated antibody that binds to p40 and one or both of IL-4, IL-13, selected from one or more of Tables 86 and 87.

E189. The antibody of any one of E144-E188, for use as a medicament.

E190. The antibody of E189, wherein the use is for the treatment of one or more selected from the group consisting of non-alcoholic steatohepatitis (NASH), psoriasis, psoriatic arthritis, atopic dermatitis, Crohn's disease, ulcerative colitis, asthma (severe), allergy, alopecia, idiopathic pulmonary fibrosis, systemic sclerosis, keloids, systemic lupus erythematosus, primary biliary cirrhosis, and hidradenitis suppurativa.

E191. The antibody of any one of E189-E190, wherein the use is for the treatment of one or more selected from the group consisting of non-alcoholic steatohepatitis (NASH), atopic dermatitis, asthma (severe), alopecia, idiopathic pulmonary fibrosis, and systemic sclerosis.

E192. The antibody of any one of E189-E191, wherein the use is for atopic dermatitis.

E193. The antibody of any one of E189-E191, wherein the use is for non-alcoholic steatohepatitis (NASH).

E194. A pharmaceutical composition comprising a therapeutically effective amount of the antibody of E144-E193 and a pharmaceutically acceptable carrier.

E195. A method of treating a medical condition, comprising administering to a subject in need thereof a therapeutically effective amount of the antibody of any one of E144-E193, or the pharmaceutical composition of E194.

E196. The method of E195, wherein the condition is selected from the group consisting of non-alcoholic steatohepatitis (NASH), psoriasis, psoriatic arthritis, atopic dermatitis, Crohn's disease, ulcerative colitis, asthma (severe), allergy, alopecia, idiopathic pulmonary fibrosis, systemic sclerosis, keloids, systemic lupus erythematosus, primary biliary cirrhosis, and hidradenitis suppurativa.

E197. The method of any one of E195-E196, comprising administering said antibody or pharmaceutical composition, subcutaneously.

E198. The method of any one of E195-E196, wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E199. An isolated antibody that specifically binds to IL-4, comprising a heavy chain variable region (IL4-VH) and a light chain variable region (IL4-VL), comprising
(i) a CDR-H1, CDR-H2, and CDR-H3 sequences of SEQ ID NO: 22, and a CDR-L1, CDR-L2, and CDR-L3 sequences of SEQ ID NO: 26
(ii) a CDR-H1, CDR-H2, and CDR-H3 sequences of SEQ ID NO: 19, and a CDR-L1, CDR-L2, and CDR-L3 sequences of SEQ ID NO: 20;
(iii) a CDR-H1, CDR-H2, and CDR-H3 sequences of SEQ ID NO: 22, and a CDR-L1, CDR-L2, and CDR-L3 sequences of SEQ ID NO: 20;
(iv) a CDR-H1, CDR-H2, and CDR-H3 sequences of SEQ ID NO: 28, and a CDR-L1, CDR-L2, and CDR-L3 sequences of SEQ ID NO: 29; or
(v) a CDR-H1, CDR-H2, and CDR-H3 sequences of SEQ ID NO: 22, and a CDR-L1, CDR-L2, and CDR-L3 sequences of SEQ ID NO: 30.

E200. An isolated antibody that specifically binds to IL-4, comprising a heavy chain variable region (IL4-VH) and a light chain variable region (IL4-VL), comprising the CDR-H1, CDR-H2, and CDR-H3 sequences of SEQ ID NO: 22, and the CDR-L1, CDR-L2, and CDR-L3 sequences of SEQ ID NO: 26.

E201. An isolated antibody that specifically binds to IL-4, comprising a heavy chain variable region (IL4-VH) and a light chain variable region (IL4-VL), wherein the CDR-H1 comprises the amino acid sequence of SEQ ID NO: 18; the CDR-H2 comprises the amino acid sequence of SEQ ID NO: 2; the CDR-H3 comprises the amino acid sequence of SEQ ID NO: 3; the CDR-L1 comprises the amino acid sequence of SEQ ID NO: 24; the CDR-L2 comprises the amino acid sequence of SEQ ID NO: 12, and the CDR-L3 comprises the amino acid sequence of SEQ ID NO: 25.

E202. The antibody of any one of E199-E201, comprising an IL4-VH framework sequence derived from a human germline VH sequence selected from the group consisting of DP26, DP27, DP28, and DP76.

E203. The antibody of any one of E199-E202, comprising an IL4-VH framework sequence derived from a human DP76 germline sequence.

E204. The antibody of any one of E199-E203, comprising an IL4-VL framework sequence derived from a human germline VL sequence selected from the group consisting of DPK1, DPK3, DPK4, DPK5, DPK7, DPK8, DPK9, and DPK24.

E205. The antibody of any one of E199-E204, comprising an IL4-VL framework sequence derived from a human germline DPK9 sequence

E206. The antibody of any one of E199-E205, comprising an IL4-VL framework sequence and an IL4-VH framework sequence, and wherein one or both of the IL4-VL framework sequence or the IL4-VH framework sequence is at least 66%, 76%, 80%, 90%, 91%, 92%, 93%, 94%, 95%, 96%, 97%, 98%, 99%, or 100% identical to the human germline sequence from which it was derived.

E207. The antibody of any one of E199-E206, comprising an IL4-VL framework sequence and an IL4-VH framework sequence, and wherein one or both of the IL4-VL framework sequence or the IL4-VH framework sequence is identical to the human germline sequence from which it was derived.

E208. The antibody of any one of E199-E207, comprising an IL4-VH sequence at least 90%, 91%, 92%, 93%, 94%, 95%, 96%, 97%, 98%, 99%, or 100% identical to SEQ ID NO: 22.

E209. The antibody of any one of E199-E208, comprising an IL4-VL sequence at least 90%, 91%, 92%, 93%, 94%, 95%, 96%, 97%, 98%, 99%, or 100% identical to SEQ ID NO: 20.

E210. The antibody of any one of E199-E209, comprising
(i) an IL4-VH sequence of SEQ ID NO: 22, and an IL4-VL of SEQ ID NO: 26;
(ii) an IL4-VH sequence of SEQ ID NO: 19, and an IL4-VL of SEQ ID NO: 20;
(iii) an IL4-VH sequence of SEQ ID NO: 22, and an IL4-VL of SEQ ID NO: 20;
(iv) an IL4-VH sequence of SEQ ID NO: 28, and an IL4-VL of SEQ ID NO: 29; or
(v) an IL4-VH sequence of SEQ ID NO: 22, and an IL4-VL of SEQ ID NO: 30.

E211. The antibody of any one of E199-E210, comprising an IL4-VH sequence identical to SEQ ID NO: 22, and an IL4-VL sequence identical to SEQ ID NO: 26.

E212. The antibody of any one of E199-E211, comprising the IL4-VH sequence encoded by a nucleic acid sequence of SEQ ID NO: 200.

E213. The antibody of any one of E199-E212, comprising the IL4-VL sequence encoded by a nucleic acid sequence of SEQ ID NO: 201.

E214. The antibody of any one of E199-E213, comprising the IL4-VH sequence encoded by the plasmid deposited at the ATCC and having ATCC Accession No. PTA-127198. E215. The antibody of any one of E199-E214, comprising the IL4-VL sequence encoded by the plasmid deposited at the ATCC and having ATCC Accession No. PTA-127197.

E216. An antibody comprising the IL4-VH sequence encoded by the plasmid deposited at the ATCC and having ATCC Accession No. PTA-127198, and the IL4-VL sequence encoded by the plasmid deposited at the ATCC and having ATCC Accession No. PTA-127197.

E217. The antibody of E199-E216, wherein the antibody binds human IL-4 with a K_{D} less than a value selected from the group consisting of about, 10 pM, 5 pM, 1 pM, and 800 fM.

E218. The antibody of E199-E217, wherein the antibody binds human IL-4 with a K_{D} less than a value of about 1 pM.

E219. The antibody of any one of E199-E218, wherein the K_{D} value is measured by kinetic exclusion assay.

E220. The antibody of E199-E219, wherein the antibody binds cynomolgus IL-4.

E221. The antibody of E199-E220, wherein the antibody does not bind IL-4 from one or more selected from the groups consisting of dog, sheep, rabbit, rat, and mouse.

E222. The antibody of E199-E221, wherein the binding K_{D} of the antibody to cynomolgus IL-4 is within 1 order of magnitude of the binding K_{D} of the antibody to human IL-4.

E223. The antibody of E199-E222, wherein the binding K_{D} of the antibody to cynomolgus IL-4 is within two-fold difference of the binding K_{D} of the antibody to human IL-4.

E224. The antibody of E199-E223, wherein the antibody is characterized by an IC₅₀ of less than 10 pM in a human monocyte assay for neutralization of IL-4 induction of CD23.

E225. The antibody of E199-E224, wherein the antibody is characterized by an IC₅₀ of less than 20 pM in a human monocyte assay for neutralization of cynomolgus monkey IL-4 induction of CD23.

E226. The antibody of E199-E225, wherein the antibody has a viscosity of 20 cP or less at 25° C. at a concentration of 80 mg/mL in a Histidine-sucrose pH 5.8 buffer.

E227. The antibody of E199-E226, wherein the antibody has a viscosity of 20 cP or less at 25° C. at a concentration of 100 mg/mL in a Histidine-sucrose pH 5.8 buffer.

E228. The antibody of E199-E227, wherein the antibody has a viscosity of 20 cP or less at 25° C. at a concentration of 120 mg/mL in a Histidine-sucrose pH 5.8 buffer.

E229. The antibody of E199-E228, wherein the antibody comprises a lysine at residue 93 in the light chain.

E230. The antibody of E199-E229, further comprising a constant heavy domain (IL4-CH1) and a constant light domain (IL4-CL).

E231. The antibody of E230, wherein the IL4-CH1 comprises a sequence according to SEQ ID NO: 6.

E232. The antibody of E230-E231, wherein the IL4-CL comprises a sequence according to SEQ ID NO: 16.

E233. The antibody of E230-E232, wherein the IL4-CH1 is connected to the IL4-VL, and the IL4-CL is connected to the IL-4-VH forming an IL-4-binding domain-swap Fab domain (IL4-xFab).

E234. The antibody of E230-E232, wherein the IL4-CH1 is connected to the IL4-VH, and the IL4-CL is connected to the IL4-VL forming an IL-4 binding Fab domain (IL4-Fab).

E235. The antibody of any one of E199-E234, comprising an Fc domain comprising a first Fc chain and a second Fc chain.

E236. The antibody of E235, wherein the Fc domain is the Fc domain of an IgA (for example IgA₁ or IgA₂), IgD, IgE, IgM, or IgG (for example IgG₁, IgG₂, IgG₃, or IgG₄).

E237. The antibody of E235-E236 wherein the Fc domain is the Fc domain of an IgG₁.

E238. The antibody, or antigen-binding fragment thereof, of E235-E237, wherein the N-terminus of the first Fc chain or the second Fc chain is connected to the C-terminus of the IL33-CH1 domain.

E239. The antibody, or antigen-binding fragment thereof, of E235-E238, wherein the first and second Fc chain each comprises, from N-terminus to C-terminus: a hinge region, a CH2 region, and a CH3 region.

E240. The antibody, or antigen-binding fragment thereof, of E239, wherein the hinge region comprises a sequence selected from the group consisting of SEQ ID NO: 7, SEQ ID NO: 102, SEQ ID NO: 123, SEQ ID NO: 126, SEQ ID NO: 129, and SEQ ID NO: 131.

E241. The antibody, or antigen-binding fragment thereof, of E240, wherein the hinge region comprises a sequence according to SEQ ID NO: 7.

E242. The antibody, or antigen-binding fragment thereof, of E240, wherein the hinge region on the first Fc chain and the hinge region on the second Fc chain comprise a pair of sequences according to SEQ ID NO: 129 and SEQ ID NO: 131.

E243. The antibody, or antigen-binding fragment thereof, of E239-E242, wherein the CH2 region comprises a sequence according to SEQ ID NO: 8.

E244. The antibody, or antigen-binding fragment thereof, of E239-E243, wherein the CH3 region on the first Fc chain and the CH3 region on the second Fc chain comprise a pair of sequences selected from the group consisting of
(i) SEQ ID NO: 9 and SEQ ID NO: 9;
(ii) SEQ ID NO: 111 and SEQ ID NO: 106;
(iii) SEQ ID NO: 111 and SEQ ID NO: 114;
(iv) SEQ ID NO: 114 and SEQ ID NO: 117;
(v) SEQ ID NO: 124 and SEQ ID NO: 127;
(vi) SEQ ID NO: 139 and SEQ ID NO: 141; and
(vii) SEQ ID NO: 147 and SEQ ID NO: 148.

E245. The antibody, or antigen-binding fragment thereof, of E244, wherein the CH3 region on the first Fc chain and the CH region on the second Fc chain each comprise a sequence according to SEQ ID NO: 9.

E246. The antibody, or antigen-binding fragment thereof, of E244, wherein the CH3 region on the first Fc chain and the CH3 region on the second Fc chain comprise a pair of sequences according to SEQ ID NO: 124 and SEQ ID NO: 127.

E247. The antibody of any one of E199-E246, comprising an IL4-VH bearing polypeptide comprising an amino acid sequence at least 90%, 91%, 92%, 93%, 94%, 95%, 96%, 97%, 98%, 99%, or 100% identical to a sequence selected from the group consisting of SEQ ID NO: 23, SEQ ID NO: 107, SEQ ID NO: 115, SEQ ID NO: 121, SEQ ID NO: 125, SEQ ID NO: 130, SEQ ID NO: 133, SEQ ID NO: 135, SEQ ID NO: 140, SEQ ID NO: 144, SEQ ID NO: 146, SEQ ID NO: 151, SEQ ID NO: 153, SEQ ID NO: 153, SEQ ID NO: 156, SEQ ID NO: 157, SEQ ID NO: 159, SEQ ID NO: 162, and SEQ ID NO: 164, SEQ ID NO: 179, SEQ ID NO: 180, and SEQ ID NO: 183.

E248. The antibody of any one of E199-E247, comprising an IL4-VH bearing polypeptide comprising the amino acid sequence of SEQ ID NO: 23.

E249. The antibody of any one of E199-E241, comprising an IL4-VH bearing polypeptide comprising the amino acid sequence of SEQ ID NO8: 130.

E250. The antibody of any one of E199-E249, comprising an IL4-VL bearing polypeptide comprising an amino acid sequence at least 90%, 91%, 92%, 93%, 94%, 95%, 96%, 97%, 98%, 99%, or 100%, identical to a sequence selected from the group consisting of SEQ ID NO: 27, SEQ ID NO: 109, and SEQ ID NO: 116, SEQ ID NO: 136, SEQ ID NO: 197, and SEQ ID NO: 208.

E251. The antibody of any one of E199-E250, comprising an IL4-VL bearing polypeptide comprising the amino acid sequence of SEQ ID NO: 27.

E252. The antibody of any one of E199-E251, comprising the IL4-VH bearing polypeptide sequence encoded by the plasmid deposited at the ATCC and having ATCC Accession No. PTA-127192.

E253. The antibody of any one of E199-E252, comprising the IL4-VL bearing polypeptide sequence encoded by the plasmid deposited at the ATCC and having ATCC Accession No. PTA-127194.

E254. The antibody comprising an IL4-VH bearing polypeptide sequence encoded by the plasmid deposited at the ATCC and having ATCC Accession No. PTA-127192, and an IL4-VL bearing polypeptide sequence encoded by the plasmid deposited at the ATCC and having ATCC Accession No. PTA-127194.

E255. The antibody of any one of E199-E254, for use as a medicament.

E256. The antibody of any one of E199-E255, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, Crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa.

E257. The antibody of any one of E199-256, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, non-alcoholic steatohepatitis (NASH), alopecia, idiopathic pulmonary fibrosis, and systemic sclerosis.

E258. The antibody of any one of E199-257, wherein the use is for atopic dermatitis. E259. The antibody of any one of E199-257, wherein the use is for non-alcoholic steatohepatitis (NASH).

E260. A pharmaceutical composition comprising a therapeutically effective amount of the antibody of E199-E259 and a pharmaceutically acceptable carrier.

E261. A method of treating a medical condition, comprising administering to a subject in need thereof a therapeutically effective amount of The antibody of any one of E199-E259, or the pharmaceutical composition of E260.

E262. The method of E261, wherein the condition is selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, diabetic kidney disease, Behcet's disease, gout, Alzheimer's disease, and atherosclerosis.

E263. The method of any one of E261-E262, comprising administering said antibody or pharmaceutical composition, subcutaneously.

E264. The method of any one of E261-E263, wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E265. An isolated antibody that specifically binds to IL-13, comprising a heavy chain variable region (IL13-VH) and a light chain variable region (IL13-VL), comprising
(i) the CDR-H1, CDR-H2, and CDR-H3 sequences of SEQ ID NO: 51, and the CDR-11, CDR-L2, and CDR-L3 sequences of SEQ ID NO: 54
(ii) the CDR-H1, CDR-H2, and CDR-H3 sequences of SEQ ID NO: 44, and the CDR-11, CDR-L2, and CDR-L3 sequences of SEQ ID NO: 46;
(iii) the CDR-H1, CDR-H2, and CDR-H3 sequences of SEQ ID NO: 48, and the CDR-11, CDR-L2, and CDR-L3 sequences of SEQ ID NO: 49;
(iv) the CDR-H1, CDR-H2, and CDR-H3 sequences of SEQ ID NO: 48, and the CDR-11, CDR-L2, and CDR-L3 sequences of SEQ ID NO: 68; or
(v) the CDR-H1, CDR-H2, and CDR-H3 sequences of SEQ ID NO: 57, and the CDR-11, CDR-L2, and CDR-L3 sequences of SEQ ID NO: 59.

E266. An isolated antibody that specifically binds to IL-13, comprising a heavy chain variable region (IL13-VH) and a light chain variable region (IL13-VL), comprising the CDR-H1, CDR-H2, and CDR-H3 sequences of SEQ ID NO: 51, and the CDR-L1, CDR-L2, and CDR-L3 sequences of SEQ ID NO: 54.

E267. An isolated antibody that specifically binds to IL-13, comprising a heavy chain variable region (IL13-VH) and a light chain variable region (IL13-VL), wherein the CDR-H1 comprises the amino acid sequence of SEQ ID NO: 41; the CDR-H2 comprises the amino acid sequence of SEQ ID NO: 42; the CDR-H3 comprises the amino acid sequence of SEQ ID NO:-50; the CDR-L1 comprises the amino acid sequence of SEQ ID NO: 53; the CDR-L2 comprises the amino acid sequence of SEQ ID NO: 37, and the CDR-L3 comprises the amino acid sequence of SEQ ID NO: 38.

E268. The antibody of any one of E265-E267, comprising an IL13-VH framework sequence derived from a human germline VH sequence selected from the group consisting of DP7, DP10, DP35, DP47, DP50, DP51, DP54, and DP77.

E269. The antibody of any one of E265-E268, comprising an IL13-VH framework sequence derived from a human DP54 germline sequence.

E270. The antibody of any one of E265-E269, comprising an IL13-VL framework sequence derived from a human germline VL sequence selected from the group consisting of DPK3, DPK4, DPK5, DPK8, DPK9, DPK10, DPK23.

E271. The antibody of any one of E265-E270, comprising an IL13-VL framework sequence derived from a human germline DPK9 sequence

E272. The antibody of any one of E265-E271, comprising an IL13-VL framework sequence and an IL13-VH framework sequence, and wherein one or both of the IL13-VL framework sequence or the IL13-VH framework sequence is at least 66%, 76%, 80%, 90%, 91%, 92%, 93%, 94%, 95%, 96%, 97%, 98%, 99%, or 100% identical to the human germline sequence from which it was derived.

E273. The antibody of any one of E265-E272, comprising an IL13-VL framework sequence and an IL13-VH framework sequence, and wherein one or both of the IL13-VL framework sequence or the IL13-VH framework sequence is identical to the human germline sequence from which it was derived.

E274. The antibody of any one of E265-E273, comprising an IL13-VH at least 90%, 91%, 92%, 93%, 94%, 95%, 96%, 97%, 98%, 99%, or 100% identical to SEQ ID NO: 51.

E275. The antibody of any one of E265-E274, comprising an IL13-VL at least 90%, 91%, 92%, 93%, 94%, 95%, 96%, 97%, 98%, 99%, or 100% identical to SEQ ID NO: 54.

E276. The antibody of any one of E265-E275, comprising
(i) the IL13-VH of SEQ ID NO: 51, and the IL13-VL of SEQ ID NO: 54
(ii) the IL13-VH of SEQ ID NO: 44, and the IL13-VL of SEQ ID NO: 46;
(iii) the IL13-VH of SEQ ID NO: 48, and the IL13-VL of SEQ ID NO: 49;
(iv) the IL13-VH of SEQ ID NO: 48, and the IL13-VL of SEQ ID NO: 68; or
(v) the IL13-VH of SEQ ID NO: 57, and the IL13-VL of SEQ ID NO: 59.

E277. The antibody of any one of E265-E276, comprising an IL13-VH identical to SEQ ID NO: 51, and an IL13-VL identical to SEQ ID NO: 54.

E278. The antibody of any one of E265-E277, comprising the VH sequence encoded a nucleic acid sequence of SEQ ID NO: 198.

E279. The antibody of any one of E265-E278, comprising the VL sequence encoded a nucleic acid sequence of SEQ ID NO: 199.

E280. The antibody of any one of E265-E279, comprising the IL13-VH sequence encoded by the plasmid deposited at the ATCC and having ATCC Accession No. PTA-127196.

E281. The antibody of any one of E265-E280, comprising the IL13-VL sequence encoded by the plasmid deposited at the ATCC and having ATCC Accession No. PTA-127195.

E282. An antibody comprising the IL13-VH sequence encoded by the plasmid deposited at the ATCC and having ATCC Accession No. PTA-127196, and the IL13-VL sequence encoded by the plasmid deposited at the ATCC and having ATCC Accession No. PTA-127195.

E283. The antibody of E265-E282, wherein the antibody binds human IL-13 with a K_{D} less than a value selected from the group consisting of 10 nM, 5 nM, 2 nM, 1 nM, 900 pM, 800 pM, 700 pM, 600 pM, 500 pM, 400 pM, 300 pM, 250 pM, 200 pM, 150 pM, 100 pM, and 60 pM.

E284. The antibody of E265-E276, wherein the antibody binds human IL-13 with a K_{D} less than 60 pM.

E285. The antibody of any one of E283-E284, wherein the K_{D} value is measured by kinetic exclusion assay.

E286. The antibody of any one of E283-E285, wherein the K_{D} value is measured by SPR.

E287. The antibody of E265-E286, wherein the antibody binds cynomolgus IL-13.

E288. The antibody of E265-E287, wherein the antibody does not bind IL-13 from one or more species selected from the group consisting of dog, rabbit, and mouse.

E289. The antibody of E265-E288, wherein the binding K_{D} of the antibody to cynomolgus IL-13 is within 1 order of magnitude of the binding K_{D} of the antibody to human IL-13.

E290. The antibody of E265-E289, wherein the binding K_{D} of the antibody to cynomolgus IL-13 is within five-fold difference of the binding K_{D} of the antibody thereof, to human IL-13.

E291. The antibody of E265-E290, wherein the binding K_{D} of the antibody to cynomolgus IL-13 is within two-fold difference of the binding K_{D} of the antibody thereof, to human IL-13.

E292. The antibody of E265-E291, wherein the IL-13 IC₅₀ is less than 100 pM as measured by neutralization of IL-13 pSTAT6 phosphorylation in HT-29 cells.

E293. The antibody of E265-E292, wherein the IL-13 IC₅₀ is less than 20 pM as measured in a human monocyte assay for neutralization of IL-13 induction of CD23.

E294. The antibody of E265-E293, wherein the IL-13 IC₅₀ is less than 15 pM as measured in a human monocyte assay for neutralization of IL-13 induction of CD23.

E295. The antibody of E265-E294, wherein the IL-13 IC₅₀ is less than 12 pM as measured in a human monocyte assay for neutralization of IL-13 induction of CD23.

E296. The antibody of E265-E295, further comprising a constant heavy domain (IL13-CH1) and a constant light domain (IL13-CL).

E297. The antibody of E296, wherein the IL13-CH1 comprises a sequence selected from the group consisting of SEQ ID NO: 6, SEQ ID NO: 105, and SEQ ID NO: 110.

E298. The antibody of E296-E297, wherein the IL13-CH1 comprises a sequence according to SEQ ID NO: 6.

E299. The antibody of E296-E298, wherein the IL13-CL comprises a sequence selected from the group consisting of SEQ ID NO: 16, SEQ ID NO: 108, and SEQ ID NO: 113.

E300. The antibody of E296-E299, wherein the IL13-CL comprises a sequence according to SEQ ID NO:16.

E301. The antibody of E296-E300, wherein the IL13-CH1 is connected to the IL13-VL, and the IL13-CL is connected to the IL13-VH forming an IL-13-binding domain-swap Fab domain (IL13-xFab).

E302. The antibody of E296-E301, wherein the IL13-CH1 is connected to the IL13-VH, and the IL13-CL is connected to the IL33-VL forming an IL-13 binding Fab domain (IL13-Fab).

E303. The antibody of any one of E265-E302, comprising an Fc domain comprising a first Fc chain and a second Fc chain.

E304. The antibody of E303, wherein the Fc domain is the Fc domain of an IgA (for example IgA₁ or IgA₂), IgD, IgE, IgM, or IgG (for example IgG₁, IgG₂, IgG₃, or IgG₄).

E305. The antibody of E303-E304 wherein the Fc domain is the Fc domain of an IgG₁.

E306. The antibody of E303-E305, wherein the N-terminus of the first Fc chain or the second Fc chain is connected to the C-terminus of the IL13-CH1 domain.

E307. The antibody of E303-E306, wherein the first Fc chain and the second Fc chain each comprises, from N-terminus to C-terminus: a hinge region, a CH2 region, and a CH3 region.

E308. The antibody of E307, wherein the hinge region comprises a sequence selected from the group consisting of SEQ ID NO: 7, SEQ ID NO: 102, SEQ ID NO: 123, SEQ ID NO:126, SEQ ID NO: 129, and SEQ ID NO:131.

E309. The antibody of E307-E308, wherein the hinge region comprises a sequence according to SEQ ID NO: 7.

E310. The antibody of E307-E309, wherein the hinge region comprises a sequence according to SEQ ID NO: 102.

E311. The antibody of E307-E310, wherein the CH2 region comprises a sequence according to SEQ ID NO: 8.

E312. The antibody of E307-E311 wherein the CH3 region on the first Fc chain and the CH3 region on the second Fc chain comprise a pair of sequences selected from the group consisting of
(i) SEQ ID NO: 124 and SEQ ID NO: 127;
(ii) SEQ ID NO: 9 and SEQ ID NO: 9;
(iii) SEQ ID NO: 111 and SEQ ID NO: 106;
(iv) SEQ ID NO: 111 and SEQ ID NO: 114;
(v) SEQ ID NO: 114 and SEQ ID NO: 117;
(vi) SEQ ID NO: 139 and SEQ ID NO: 141; and
(vii) SEQ ID NO: 147 and SEQ ID NO: 148.

E313. The antibody of E307-E312, wherein the CH3 region on the first Fc chain and the CH region on the second Fc chain each comprise a sequence according to SEQ ID NO: 9.

E314. The antibody of E307-E312, wherein the CH3 region on the first Fc chain and the CH region on the second Fc chain comprise a pair of sequences according to SEQ ID NO: 124 and SEQ ID NO: 127.

E315. The antibody of any one of E265-E314, comprising an IL13-VH bearing polypeptide comprising an amino acid sequence at least 90%, 91%, 92%, 93%, 94%, 95%, 96%, 97%, 98%, 99%, or 100% identical to a sequence selected from the group consisting of SEQ ID NO: 52, SEQ ID NO:66, SEQ ID NO: 112, SEQ ID NO: 118, SEQ ID NO: 121, SEQ ID NO: 122, SEQ ID NO: 130, SEQ ID NO: 145, SEQ ID NO: 149, SEQ ID NO: 152, SEQ ID NO: 154, SEQ ID NO:160, SEQ ID NO: 162, and SEQ ID NO: 164, and SEQ ID NO: 209.

E316. The antibody of any one of E265-E315, comprising an IL13-VH bearing polypeptide consisting of SEQ ID NO: 52, SEQ ID NO:66, SEQ ID NO: 112, SEQ ID NO: 118, SEQ ID NO: 121, SEQ ID NO: 122, SEQ ID NO: 130, SEQ ID NO: 145, SEQ ID NO: 149, SEQ ID NO: 152, SEQ ID NO: 154, SEQ ID NO:160, SEQ ID NO: 162, and SEQ ID NO: 164, and SEQ ID NO: 209.

E317. The antibody of any one of E264-E316, comprising an IL13-VH bearing polypeptide comprising the amino acid sequence of SEQ ID NO: 52.

E318. The antibody of any one of E265-E316, comprising an IL13-VH bearing polypeptide comprising the amino acid sequence of SEQ ID NO: 122.

E319. The antibody of any one of E265-E318, comprising an IL13-VL bearing polypeptide comprising an amino acid sequence at least 90%, 91%, 92%, 93%, 94%, 95%, 96%, 97%, 98%, 99%, or 100% identical to a sequence selected from the group consisting of SEQ ID NO: 55, SEQ ID NO: 163, and SEQ ID NO: 196.

E320. The antibody of any one of E265-E319, comprising an IL13-VL bearing polypeptide comprising an amino acid sequence selected from the group consisting of SEQ ID NO: 55, SEQ ID NO: 119, SEQ ID NO: 120, SEQ ID NO: 125, SEQ ID NO: 130, SEQ ID NO: 133, SEQ ID NO: 135, SEQ ID NO: 140, SEQ ID NO: 163, SEQ ID NO: 164, and SEQ ID NO: 196.

E321. The antibody of any one of E265-E320, comprising an IL13-VL bearing polypeptide comprising the amino acid sequence of SEQ ID NO: 55.

E322. The antibody of any one of E265-E321, comprising an IL13-VL bearing polypeptide comprising the amino acid sequence of SEQ ID NO: 130.

E323. The antibody of any one of E265-E322, comprising the IL13-VH bearing polypeptide sequence encoded by the plasmid deposited at the ATCC and having ATCC Accession No. PTA-127193.

E324. The antibody of any one of E265-E323, comprising the IL13-VL bearing polypeptide sequence encoded by the plasmid deposited at the ATCC and having ATCC Accession No. PTA-127192.

E325. An antibody comprising the IL13-VH polypeptide sequence encoded by the plasmid deposited at the ATCC and having ATCC Accession No. PTA-127193, and the IL13-VL bearing polypeptide sequence encoded by the plasmid deposited at the ATCC and having ATCC Accession No. PTA-127192.

E326. The antibody of any one of E265-E325, for use as a medicament.

E327. The antibody of E326, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, Crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa.

E328. The antibody of any one of E326-E327, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, non-alcoholic steatohepatitis (NASH), alopecia, idiopathic pulmonary fibrosis, and systemic sclerosis.

E329. The antibody of any one of E326-E328, wherein the use is for atopic dermatitis.

E330. The antibody of any one of E326-E329, wherein the use is for non-alcoholic steatohepatitis (NASH).

E331. A pharmaceutical composition comprising a therapeutically effective amount of the antibody of E265-E330 and a pharmaceutically acceptable carrier.

E332. A method of treating a medical condition, comprising administering to a subject in need thereof a therapeutically effective amount of the antibody of any one of E265-E330, or the pharmaceutical composition of E331.

E333. The method of E332, wherein the condition is selected from the group consisting of non-alcoholic steatohepatitis (NASH), psoriasis, psoriatic arthritis, atopic dermatitis, Crohn's disease, ulcerative colitis, asthma (severe), allergy, alopecia, idiopathic pulmonary fibrosis, systemic sclerosis, keloids, systemic lupus erythematosus, primary biliary cirrhosis, and hidradenitis suppurativa.

E334. The method of any one of E332-E333, comprising administering said antibody or pharmaceutical composition, subcutaneously.

E335. The method of any one of E332-E334, wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E336. An isolated antibody that specifically binds to IL-33, specifically binds to IL-4, and specifically binds to IL-13, comprising an IL-33 binding domain, an IL-4 binding domain, and an IL-13 binding domain.

E337. The antibody of E336, wherein the specific binding to IL-33 is through an antibody of any one of E1-E71.

E338. The antibody of E336-E337, wherein the specific binding to IL-4 is through an antibody of any one of E199-E259.

E339. The antibody of any one of E336-E338, wherein the specific binding to IL-13 is through an antibody of any one of E265-E330.

E340. The antibody of any one of E336-E339, wherein
(i) the IL-33 binding domain comprises a heavy chain variable region (IL33-VH) and a light chain variable region (IL33-VL), wherein the CDR-H1 of the IL-33 binding domain comprises the amino acid sequence of SEQ ID NO: 60; the CDR-H2 of the IL-33 binding domain comprises the amino acid sequence of SEQ ID NO: 61; the CDR-H3 of the IL-33 binding domain comprises the amino acid sequence of SEQ ID NO: 72; the CDR-L1 of the IL-33 binding domain comprises the amino acid sequence of SEQ ID NO: 75; the CDR-L2 of the IL-33 binding domain comprises the amino acid sequence of SEQ ID NO: 76, and the CDR-L3 of the IL-33 binding domain comprises the amino acid sequence of SEQ ID NO: 77; and
(ii) the IL-4 binding domain comprises a heavy chain variable region (IL4-VH) and a light chain variable region (IL4-VL), wherein the CDR-H1 of the IL-4 binding domain comprises the amino acid sequence of SEQ ID NO: 18; the CDR-H2 of the IL-4 binding domain comprises the amino acid sequence of SEQ ID NO: 2; the CDR-H3 of the IL-4 binding domain comprises the amino acid sequence of SEQ ID NO: 3; the CDR-L1 of the IL-4 binding domain comprises the amino acid sequence of SEQ ID NO: 24; the CDR-L2 of the IL-4 binding domain comprises the amino acid sequence of SEQ ID NO: 12, and the CDR-L3 of the IL-4 binding domain comprises the amino acid sequence of SEQ ID NO: 25, and
(iii) the IL-13 binding domain comprises a heavy chain variable region (IL13-VH) and a light chain variable region (IL13-VL), wherein the CDR-H1 of the IL-13 binding domain comprises the amino acid sequence of SEQ ID NO: 41; the CDR-H2 of the IL-13 binding domain comprises the amino acid sequence of SEQ ID NO: 42; the CDR-H3 of the IL-13 binding domain comprises the amino acid sequence of SEQ ID NO:-50; the CDR-L1 of the IL-13 binding domain comprises the amino acid sequence of SEQ ID NO: 53; the CDR-L2 of the IL-13 binding domain comprises the amino acid sequence of SEQ ID NO: 37, and the CDR-L3 of the IL-13 binding domain comprises the amino acid sequence of SEQ ID NO: 38.

E341. The antibody of E336-E340, wherein
(i) the IL-33 binding domain comprises an IL33-VH of SEQ ID NO: 73, and an IL33-VL of SEQ ID NO: 78;
(i) the IL-4 binding domain comprises an IL4-VH of SEQ ID NO: 22, and an IL4-VL of SEQ ID NO: 26; and
(ii) the IL-13 binding domain comprises an IL13-VH of SEQ ID NO: 51, and an IL13-VL of SEQ ID NO: 54.

E342. The antibody of E336-341, wherein
(i) the IL-33 binding domain comprises an IL33-VH sequence encoded by the plasmid deposited at the ATCC and having ATCC Accession No. PTA-127210, and an IL33-VL sequence encoded by the plasmid deposited at the ATCC and having ATCC Accession No. PTA-127209.
(ii) the IL-4 binding domain comprises IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) the IL-13 binding domain comprises an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

E343. The antibody of E336-E342, wherein the IL-33 binding domain is fused with or without a linker to the IL-13 binding domain.

E344. The antibody of E336-E342, wherein the IL-33 binding domain is fused with or without a linker to the IL-4 binding domain.

E345. The antibody of E336-E342, wherein the IL-13 binding domain is fused with or without a linker to the IL-4 binding domain.

E346. The antibody of any one of E343-E345, wherein the fusion is with a linker.

E347. The antibody of any one of E336-E346, wherein the IL-13 binding domain is fused with a linker to the IL-4 binding domain.

E348. The antibody of E343-E347, wherein the linker comprises SEQ ID NO: 104.

E349. The antibody of any one of E336-E348, wherein the antibody comprises a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site.

E350. The antibody of E349, wherein the first and second polypeptide chains associate together to form an antibody comprising two arms; a dual Fab arm comprising the first Fab domain and the second Fab domain, and a single Fab arm comprising the third Fab domain.

E351. The antibody of E349-E350, wherein the fifth polypeptide chain comprises the sequence EPKSC (SEQ ID NO: 122) at the C-terminus.

E352. The antibody of E349-E351, wherein the first antigen binding site specifically binds IL-13, the second antibody binding site specifically binds IL-4, and the third antigen binding site specifically binds IL-33.

E353. The antibody of E349-E352, wherein the first Fab domain, second Fab domain, and third Fab domain each comprise a different option selected from (i), (ii), and (iii) as follows:
(i) the IL33-Fab of E38;
(ii) the IL4-Fab of E234; and
(iii) the IL13-Fab of E302.

E354. The antibody of E349-E353, wherein the first Fab domain is the IL13-Fab of E302, the second Fab domain is the IL4-Fab of E234, and the third Fab domain is the IL33-Fab of E38.

E355. The antibody of E349-E354, wherein the first polypeptide comprises a first Fc chain, and the second polypeptide comprises a second Fc chain.

E356. The antibody of E355, wherein the first Fc chain and the second Fc chain each contain one or more amino acid modifications that promote the association of the first Fc chain with the second Fc chain.

E357. The antibody of E349-E356, wherein the first Fc chain comprises a first CH3 domain, and the second Fc chain comprises a second CH3 domain, and the first CH3 domain and the second CH3 domain each comprise a different and complementary sequence, and the different and complementary sequences are selected from one of the following pairs of different and complementary sequences:
(i) SEQ ID NO: 111 and SEQ ID NO: 106;
(ii) SEQ ID NO: 111 and SEQ ID NO: 114;
(iii) SEQ ID NO: 114 and SEQ ID NO: 117;
(iv) SEQ ID NO: 124 and SEQ ID NO: 127;
(v) SEQ ID NO: 139 and SEQ ID NO: 141; and
(vi) SEQ ID NO: 147 and SEQ ID NO: 148.

E358. The antibody of E357, wherein the first CH3 domain and the second CH3 domain comprise SEQ ID NO: 124 and SEQ ID NO: 127.

E359. The antibody of E349-E358, wherein the identity of the first, second, third, fourth, and fifth polypeptide chains is selected from the group consisting of
(i) the first polypeptide chain comprises SEQ ID NO: 132, the second polypeptide chain comprises SEQ ID NO: 130, the third polypeptide chain comprises SEQ ID NO: 79, the fourth polypeptide chain comprises SEQ ID NO: 27, and the fifth polypeptide chain comprises SEQ ID NO: 122;
(ii) the first polypeptide chain comprises SEQ ID NO: 146, the second polypeptide chain comprises SEQ ID NO: 145, the third polypeptide chain comprises SEQ ID NO: 109, the fourth polypeptide chain comprises SEQ ID NO: 196, and the fifth polypeptide chain comprises SEQ ID NO: 103;
(iii) the first polypeptide chain comprises SEQ ID NO: 112, the second polypeptide chain comprises SEQ ID NO: 107, the third polypeptide chain comprises SEQ ID NO: 196, the fourth polypeptide chain comprises SEQ ID NO: 109, and the fifth polypeptide chain comprises SEQ ID NO: 103;
(iv) the first polypeptide chain comprises SEQ ID NO: 118, the second polypeptide chain comprises SEQ ID NO 115, the third polypeptide chain comprises SEQ ID NO: 119, the fourth polypeptide chain comprises SEQ ID NO: 116, and the fifth polypeptide chain comprises SEQ ID NO: 103;
(v) the first polypeptide chain comprises SEQ ID NO: 118, the second polypeptide chain comprises SEQ ID NO: 115, the third polypeptide chain comprises SEQ ID NO: 120, the fourth polypeptide chain comprises SEQ ID NO: 116, and the fifth polypeptide chain comprises SEQ ID NO: 103;
(vi) the first polypeptide chain comprises SEQ ID NO: 209, the second polypeptide chain comprises SEQ ID NO: 121, the third polypeptide chain comprises SEQ ID NO: 196, the fourth polypeptide chain comprises SEQ ID NO: 109, and the fifth polypeptide chain comprises SEQ ID NO: 103;
(vii) the first polypeptide chain comprises SEQ ID NO: 128, the second polypeptide chain comprises SEQ ID NO: 125, the third polypeptide chain comprises SEQ ID NO: 79, the fourth polypeptide chain comprises SEQ ID NO: 208, and the fifth polypeptide chain comprises SEQ ID NO: 122;
(viii) the first polypeptide chain comprises SEQ ID NO: 134, the second polypeptide chain comprises SEQ ID NO: 133, the third polypeptide chain comprises SEQ ID NO: 79, the fourth polypeptide chain comprises SEQ ID NO: 27, and the fifth polypeptide chain comprises SEQ ID NO: 122;
(ix) the first polypeptide chain comprises SEQ ID NO: 121, the second polypeptide chain comprises SEQ ID NO: 144, the third polypeptide chain comprises SEQ ID NO: 196, the fourth polypeptide chain comprises SEQ ID NO: 136, and the fifth polypeptide chain comprises SEQ ID NO: 143;
(x) the first polypeptide chain comprises SEQ ID NO: 137, the second polypeptide chain comprises SEQ ID NO: 135, the third polypeptide chain comprises SEQ ID NO: 138, the fourth polypeptide chain comprises SEQ ID NO: 136, and the fifth polypeptide chain comprises SEQ ID NO: 122;
(xi) the first polypeptide chain comprises SEQ ID NO: 142, the second polypeptide chain comprises SEQ ID NO: 140, the third polypeptide chain comprises SEQ ID NO: 79, the fourth polypeptide chain comprises SEQ ID NO: 27, and the fifth polypeptide chain comprises SEQ ID NO: 122.

E360. The antibody of any one of E349-E359, wherein the first polypeptide chain comprises SEQ ID NO: 132, the second polypeptide chain comprises SEQ ID NO: 130, the third polypeptide chain comprises SEQ ID NO: 79, the fourth polypeptide chain comprises SEQ ID NO: 27, and the fifth polypeptide chain comprises SEQ ID NO: 122.

E361. An isolated antibody that specifically binds IL-33, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an IL-33 binding site, and
wherein the first polypeptide chain comprises SEQ ID NO: 132, the second polypeptide chain comprises SEQ ID NO: 130, the third polypeptide chain comprises SEQ ID NO: 79, the fourth polypeptide chain comprises SEQ ID NO: 27, and the fifth polypeptide chain comprises SEQ ID NO: 122.

E362. An isolated antibody that specifically binds IL-33, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an IL-33 binding site, and
wherein the first polypeptide chain comprises a sequence encoded by the plasmid deposited at the ATCC and having ATCC Accession No. PTA-127208; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127207; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

E363. The antibody of E336-E362, wherein the antibody has a viscosity of less than 20 cP at concentrations of at least 50 mg/mL in a buffer of 20 mM Histidine, 8.5% sucrose, 0.05 mg/mL EDTA pH 6.0.

E364. The antibody of E336-E363, wherein the antibody has a viscosity of less than 15 cP at concentrations of at least 90 mg/mL in a buffer of 20 mM Histidine, 8.5% sucrose, 0.05 mg/mL EDTA pH 6.0.

E365. The antibody of E336-E364, wherein the antibody has a terminal half-life of at least 12 day in cynomolgus monkeys.

E366. The antibody of E330-E365, wherein the antibody has a terminal half-life of at least 16 days in TG32 mice.

E367. The antibody of E330-E366, wherein the antibody binds human IL-4 with a binding affinity of less than 220 nM as measured by SPR.

E368. The antibody of E330-E367, wherein the antibody binds human IL-13 with a binding affinity of less than 220 nM as measured by SPR.

E369. The antibody of E336-E368, wherein the antibody binds to human IL-4 with a binding affinity of less than 1 pM, as measured by KinExA in a fixed antigen assay in PBS.

E370. The antibody of E336-E369, wherein the antibody binds to cynomolgus IL-4 with a binding affinity of less than 5 pM, as measured by KinExA in a fixed antigen assay in PBS.

E371. The antibody of E336-E370, wherein the antibody binds to human IL-13 with a binding affinity of less than 1 pM, as measured by KinExA in a fixed antigen assay in PBS.

E372. The antibody of E336-E371, wherein the antibody binds to cynomolgus IL-13 with a binding affinity of less than 1 pM, as measured by KinExA in a fixed antigen assay in PBS.

E373. The antibody of E336-E372, wherein the antibody is characterized by an IC₅₀ of less than 20 nM in a human monocyte assay for neutralization of IL-4 induction of CD23.

E374. The antibody of E336-E373, wherein the antibody is characterized by an IC₅₀ of less than 20 nM in a human monocyte assay for neutralization of IL-13 induction of CD23.

E375. The antibody of E336-E374, wherein the antibody is characterized by an IC₅₀ of less than 30 nM in a wild-type IL-33 neutralization HEK-Blue SEAP assay.

E376. The antibody of E336-E375, wherein the antibody is characterized by an IC₅₀ of less than 15 pM in a recombinant constitutively active IL-33 neutralization HEK-Blue SEAP assay.

E377. The antibody of any one of E336-E376, for use as a medicament.

E378. The antibody of E377, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, diabetic kidney disease, Behcet's disease, gout, Alzheimer's disease, and atherosclerosis.

E379. The antibody of any one of E377-378, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, and non-alcoholic steatohepatitis (NASH).

E380. The antibody of any one of E377-E379, wherein the use is for atopic dermatitis.

E381. The antibody of any one of E377-E379, wherein the use is for non-alcoholic steatohepatitis (NASH).

E382. A pharmaceutical composition comprising a therapeutically effective amount of the antibody of E336-E381 and a pharmaceutically acceptable carrier.

E383. A method of treating a medical condition, comprising administering to a subject in need thereof a therapeutically effective amount of the antibody of any one of E336-E381, or the pharmaceutical composition of E382.

E384. The method of E383, wherein the condition is selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, diabetic kidney disease, Behcet's disease, gout, Alzheimer's disease, and atherosclerosis.

E385. The method of any one of E383-E384, comprising administering said antibody or pharmaceutical composition, subcutaneously.

E386. The method of any one of E383-E385, wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E387. An isolated antibody that specifically binds to TSLP, specifically binds to IL-4, and specifically binds to IL-13, comprising a TSLP binding domain, an IL-4 binding domain, and an IL-13 binding domain.

E388. The antibody of E387, wherein the specific binding to TSLP is through an antibody of any one of E77-E143.

E389. The antibody of E387-E388, wherein the specific binding to IL-4 is through an antibody of any one of E199-E259.

E390. The antibody of any one of E387-E389, wherein the specific binding to IL-13 is through an antibody of any one of E265-E330.

E391. The antibody of any one of E381-E384, wherein
(i) the TSLP binding domain comprises a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 90; and
(ii) the IL-4 binding domain comprises a heavy chain variable region (IL4-VH) and a light chain variable region (IL4-VL), wherein the CDR-H1 comprises the amino acid sequence of SEQ ID NO: 18; the CDR-H2 comprises the amino acid sequence of SEQ ID NO: 2; the CDR-H3 comprises the amino acid sequence of SEQ ID NO: 3; the CDR-L1 comprises the amino acid sequence of SEQ ID NO: 24; the CDR-L2 comprises the amino acid sequence of SEQ ID NO: 12, and the CDR-L3 comprises the amino acid sequence of SEQ ID NO: 25, and
(iii) the IL-13 binding domain comprises a heavy chain variable region (IL13-VH) and a light chain variable region (IL13-VL), wherein the CDR-H1 comprises the amino acid sequence of SEQ ID NO: 41; the CDR-H2 comprises the amino acid sequence of SEQ ID NO: 42; the CDR-H3 comprises the amino acid sequence of SEQ ID NO:-50; the CDR-L1 comprises the amino acid sequence of SEQ ID NO: 53; the CDR-L2 comprises the amino acid sequence of SEQ ID NO: 37, and the CDR-L3 comprises the amino acid sequence of SEQ ID NO: 38.

E392. The antibody of E387-E391, wherein
(i) the TSLP binding portion comprises the TSLP-VH of SEQ ID NO: 92, and the TSLP-VL of SEQ ID NO: 94;
(ii) the IL-4 binding portion comprises a IL4-VH of SEQ ID NO: 22, and a IL4-VL of SEQ ID NO: 26; and
(iii) the IL-13 binding portion comprises the IL13-VH of SEQ ID NO: 51, and the IL13-VL of SEQ ID NO: 54.

E393. The antibody of E387-E392, wherein
(i) the TSLP binding domain comprises a TSLP-VH sequence encoded by the plasmid deposited at the ATCC and having ATCC Accession No. PTA-127200, and a TSLP-VL sequence encoded by the plasmid deposited at the ATCC and having ATCC Accession No. PTA0-127199.
(ii) the IL-4 binding domain comprises IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) the IL-13 binding domain comprises an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

E394. The antibody of E387-E393 wherein the TSLP binding domain is fused with or without a linker to the IL-13 binding domain.

E395. The antibody of E387-E393, wherein the TSLP binding domain is fused with or without a linker to the IL-4 binding domain.

E396. The antibody of E387-E393, wherein the IL-13 binding domain is fused with or without a linker to the IL-4 binding domain.

E397. The antibody of any one of E394-E396, wherein the fusion is with a linker.

E398. The antibody of any one of E394-E397, wherein the IL-13 binding domain is fused with a linker to the IL-4 binding domain.

E399. The antibody of E394-E398, wherein the linker comprises SEQ ID NO: 104.

E400. The antibody of any one of E387-E399, wherein the antibody comprises a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site.

E401. The antibody of E400, wherein the first and second polypeptide chains associate together to form an antibody comprising two arms; a dual Fab arm comprising the first Fab domain and the second Fab domain, and a single Fab arm comprising the third Fab domain.

E402. The antibody of E400-E401, wherein the fifth polypeptide chain comprises the sequence EPKSC (SEQ ID NO: 122) at the C-terminus.

E403. The antibody of E400-E402, wherein the first antigen binding site specifically binds IL-13, the second antibody binding site specifically binds IL-4, and the third antigen binding site specifically binds TSLP.

E404. The antibody of E400-E403, wherein the first Fab domain, second Fab domain, and third Fab domain each comprise a different option selected from (i), (ii), and (iii) as follows:
(i) the TSLP-Fab of E117;
(ii) the IL4-Fab of E234; and
(iii) the IL13-Fab of E302.

E405. The antibody of E400-E406, wherein the first Fab domain is the IL13-Fab of E302, the second Fab domain is the IL4-Fab of E234, and the third Fab domain is the TSLP-Fab of E117.

E406. The antibody of E400-E405, wherein the first polypeptide comprises a first Fc chain, and the second polypeptide comprises a second Fc chain.

E407. The antibody of E406, wherein the first Fc chain and the second Fc chain each contain one or more amino acid modifications that promote the association of the first Fc chain with the second Fc chain.

E408. The antibody of E400-E407, wherein the first Fc chain comprises a first CH3 domain, and the second Fc chain comprises a second CH3 domain, and the first CH3 domain and the second CH3 domain each comprise a different and complementary sequence, and the different and complementary sequences are selected from one of the following pairs of different and complementary sequences:
(i) SEQ ID NO: 111 and SEQ ID NO: 106;
(ii) SEQ ID NO: 111 and SEQ ID NO: 114;
(iii) SEQ ID NO: 114 and SEQ ID NO: 117;
(iv) SEQ ID NO: 124 and SEQ ID NO: 127;
(v) SEQ ID NO: 139 and SEQ ID NO: 141; and
(vi) SEQ ID NO: 147 and SEQ ID NO: 148.

E409. The antibody of E408, wherein the first CH3 domain and the second CH3 domain comprise SEQ ID NO: 124 and SEQ ID NO: 127.

E410. The antibody of E400-E409, wherein the identity of the first, second, third, fourth, and fifth polypeptide chains is selected from the group consisting of
(i) the first polypeptide chain comprises SEQ ID NO: 165, the second polypeptide chain comprises SEQ ID NO: 130, the third polypeptide chain comprises SEQ ID NO: 99, the fourth polypeptide chain comprises SEQ ID NO: 27, and the fifth polypeptide chain comprises SEQ ID NO: 122;
(ii) the first polypeptide chain comprises SEQ ID NO: 146, the second polypeptide chain comprises SEQ ID NO: 149, the third polypeptide chain comprises SEQ ID NO: 109, the fourth polypeptide chain comprises SEQ ID NO: 196, and the fifth polypeptide chain comprises SEQ ID NO: 150;
(iii) the first polypeptide chain comprises SEQ ID NO: 112, the second polypeptide chain comprises SEQ ID NO: 151, the third polypeptide chain comprises SEQ ID NO: 196, the fourth polypeptide chain comprises SEQ ID NO: 109, and the fifth polypeptide chain comprises SEQ ID NO: 150;
(iv) the first polypeptide chain comprises SEQ ID NO: 112, the second polypeptide chain comprises SEQ ID NO 159, the third polypeptide chain comprises SEQ ID NO: 196, the fourth polypeptide chain comprises SEQ ID NO: 109, and the fifth polypeptide chain comprises SEQ ID NO: 150;
(v) the first polypeptide chain comprises SEQ ID NO: 161, the second polypeptide chain comprises SEQ ID NO: 162, the third polypeptide chain comprises SEQ ID NO: 98, the fourth polypeptide chain comprises SEQ ID NO: 197, and the fifth polypeptide chain comprises SEQ ID NO: 163;
(vi) the first polypeptide chain comprises SEQ ID NO: 146, the second polypeptide chain comprises SEQ ID NO: 154, the third polypeptide chain comprises SEQ ID NO: 109, the fourth polypeptide chain comprises SEQ ID NO: 196, and the fifth polypeptide chain comprises SEQ ID NO: 155;
(vii) the first polypeptide chain comprises SEQ ID NO: 112, the second polypeptide chain comprises SEQ ID NO: 156, the third polypeptide chain comprises SEQ ID NO: 196, the fourth polypeptide chain comprises SEQ ID NO: 109, and the fifth polypeptide chain comprises SEQ ID NO: 155;
(viii) the first polypeptide chain comprises SEQ ID NO: 146, the second polypeptide chain comprises SEQ ID NO: 152, the third polypeptide chain comprises SEQ ID NO: 109, the fourth polypeptide chain comprises SEQ ID NO: 196, and the fifth polypeptide chain comprises SEQ ID NO: 98;
(ix) the first polypeptide chain comprises SEQ ID NO: 112, the second polypeptide chain comprises SEQ ID NO: 153, the third polypeptide chain comprises SEQ ID NO: 196, the fourth polypeptide chain comprises SEQ ID NO: 109, and the fifth polypeptide chain comprises SEQ ID NO: 98;
(x) the first polypeptide chain comprises SEQ ID NO: 112, the second polypeptide chain comprises SEQ ID NO: 157, the third polypeptide chain comprises SEQ ID NO: 196, the fourth polypeptide chain comprises SEQ ID NO: 109, and the fifth polypeptide chain comprises SEQ ID NO: 158;
(xi) the first polypeptide chain comprises SEQ ID NO: 146, the second polypeptide chain comprises SEQ ID NO: 160, the third polypeptide chain comprises SEQ ID NO: 109, the fourth polypeptide chain comprises SEQ ID NO: 196, and the fifth polypeptide chain comprises SEQ ID NO: 158; and
(xii) the first polypeptide chain comprises SEQ ID NO: 161, the second polypeptide chain comprises SEQ ID NO: 164, the third polypeptide chain comprises SEQ ID NO: 98, the fourth polypeptide chain comprises SEQ ID NO: 197, and the fifth polypeptide chain comprises SEQ ID NO: 122;
(xiii) the first polypeptide chain comprises SEQ ID NO: 165, the second polypeptide chain comprises SEQ ID NO: 130, the third polypeptide chain comprises SEQ ID NO: 215, the fourth polypeptide chain comprises SEQ ID NO: 27, and the fifth polypeptide chain comprises SEQ ID NO: 122; and
(xiv) the first polypeptide chain comprises SEQ ID NO: 165, the second polypeptide chain comprises SEQ ID NO: 130, the third polypeptide chain comprises SEQ ID NO: 216, the fourth polypeptide chain comprises SEQ ID NO: 27, and the fifth polypeptide chain comprises SEQ ID NO: 122.

E411. The antibody of any one of E411-E410, wherein the first polypeptide chain comprises SEQ ID NO: 165, the second polypeptide chain comprises SEQ ID NO: 130, the third polypeptide chain comprises SEQ ID NO: 99, the fourth polypeptide chain comprises SEQ ID NO: 27, and the fifth polypeptide chain comprises SEQ ID NO: 122.

E412. An isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an TSLP binding site; and
wherein the first polypeptide chain comprises SEQ ID NO: 165, the second polypeptide chain comprises SEQ ID NO: 130, the third polypeptide chain comprises SEQ ID NO: 99, the fourth polypeptide chain comprises SEQ ID NO: 27, and the fifth polypeptide chain comprises SEQ ID NO: 122.

E413. An isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a TSLP binding site, and
wherein the first polypeptide chain comprises a sequence encoded by the plasmid deposited at the ATCC and having ATCC Accession No. PTA-127202; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127201; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

E414. The antibody of any one of E387-E413, wherein the antibody has a terminal half-life of at least 14 days in cynomolgus monkeys.

E415. The antibody of any one of E387-E414, wherein the antibody has a terminal half-life of at least 18 days in TG32 mice.

E416. The antibody of any one of E387-E415, characterized by an anti-TSLP bioactivity of an IC₅₀ of less than 10 pM as measured a TARC production bioassay in human primary PBMCs.

E417. The antibody of any one of E387-E416, characterized by viscosity of 20 cP at a concentration of at least 100 mg/mL in a buffer of 20 mM Histidine, 8.5% sucrose, 0.05 mg/mL EDTA pH 6.0.

E418. The antibody of any one of E387-E417, characterized by a score of less than 2% high molecular mass species when determined by analytical size-exclusion chromatography (aSEC).

E419. The antibody of any one of E387-E418, characterized by a score of less than 12 in an affinity-capture self-interaction nanoparticle spectroscopy (AC SINS) assay.

E420. The antibody of any one of E387-E419, wherein the antibody binds to human IL-4 with a binding affinity of less than 1 pM, as measured by KinExA in a fixed antigen assay in PBS.

E421. The antibody of any one of E387-E420, wherein the antibody binds to cynomolgus IL-4 with a binding affinity of less than 1 pM, as measured by KinExA in a fixed antigen assay in PBS.

E422. The antibody of any one of E387-E421, wherein the antibody binds to human IL-13 with a binding affinity of less than 1 pM, as measured by KinExA in a fixed antigen assay in PBS.

E423. The antibody of any one of E387-E422, wherein the antibody binds to cynomolgus IL-13 with a binding affinity of less than 1 pM, as measured by KinExA in a fixed antigen assay in PBS.

E424. The antibody of any one of E387-E423, wherein the antibody binds to human TSLP with a binding affinity of less than 5 pM, as measured by KinExA in a fixed antigen assay in PBS.

E425. The antibody of any one of E387-E424, wherein the antibody binds to cynomolgus IL-13 with a binding affinity of less than 20 pM, as measured by KinExA in a fixed antigen assay in PBS.

E426. The antibody of any one of E387-E425, wherein the antibody is characterized by an IC₅₀ of less than 25 pM in a human monocyte assay for neutralization of IL-4 induction of CD23.

E427. The antibody of any one of E387-E426, wherein the antibody is characterized by an IC₅₀ of less than 15 pM a human monocyte assay for neutralization of IL-4 induction of CD23.

E428. The antibody of any one of E387-E427, wherein the antibody is characterized by an IC₅₀ of less than 60 pM in a human monocyte assay for neutralization of cynomolgus monkey IL-4 induction of CD23.

E429. The antibody of any one of E387-E428, wherein the antibody is characterized by an IC₅₀ of less than 15 pM in a human TSLP neutralization in a TARC production bioassay in human primary PBMCs.

E430. The antibody of any one of E387-E429, wherein the antibody is characterized by an IC₅₀ of less than 35 pM in a cynomolgus TSLP neutralization assay.

E431. The antibody of any one of E387-430, for use as a medicament.

E432. The antibody of E431, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis.

E433. The antibody of any one of E431-E432, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, and non-alcoholic steatohepatitis (NASH)

E434. The antibody of any one of E431-433, wherein the use is for atopic dermatitis.

E435. The antibody of any one of E431-433, wherein the use is for non-alcoholic steatohepatitis (NASH).

E436. A pharmaceutical composition comprising a therapeutically effective amount of the antibody of E387-E435 and a pharmaceutically acceptable carrier.

E437. A method of treating a medical condition, comprising administering to a subject in need thereof a therapeutically effective amount of the antibody of any one of E387-E435, or the pharmaceutical composition of E436.

E438. The method of E437, wherein the condition is selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis.

E439. The method of any one of E437-E438, comprising administering said antibody or pharmaceutical composition, subcutaneously.

E440. The method of any one of E437-E439, wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E445. An isolated antibody that specifically binds to p40, specifically binds to IL-4, and specifically binds to IL-13, comprising a p40 binding domain, an IL-4 binding domain, and an IL-13 binding domain.

E446. The antibody of E445, wherein the specific binding to p40 is through an antibody of any one of E144-E193.

E447. The antibody of E445-E446, wherein the specific binding to IL-4 is through an antibody of any one of E199-E259.

E448. The antibody of any one of E445-E447, wherein the specific binding to IL-13 is through an antibody of any one of E265-E330.

E449. The antibody of any one of E445-E448, wherein
(i) the p40 binding domain comprises a heavy chain variable region (p40-VH) and a light chain variable region (p40-VL), wherein the CDR-H1 of the p40 binding domain comprises the amino acid sequence of SEQ ID NO: 166; the CDR-H2 of the p40 binding domain comprises the amino acid sequence of SEQ ID NO: 167; the CDR-H3 of the p40 binding domain comprises the amino acid sequence of SEQ ID NO: 168; the CDR-L1 of the p40 binding domain comprises the amino acid sequence of SEQ ID NO: 171; the CDR-L2 of the p40 binding domain comprises the amino acid sequence of SEQ ID NO: 172, and the CDR-L3 of the p40 binding domain comprises the amino acid sequence of SEQ ID NO: 173; and
(ii) the IL-4 binding domain comprises a heavy chain variable region (IL4-VH) and a light chain variable region (IL4-VL), wherein the CDR-H1 of the IL-4 binding domain comprises the amino acid sequence of SEQ ID NO: 18; the CDR-H2 of the IL-4 binding domain comprises the amino acid sequence of SEQ ID NO: 2; the CDR-H3 of the IL-4 binding domain comprises the amino acid sequence of SEQ ID NO: 3; the CDR-L1 of the IL-4 binding domain comprises the amino acid sequence of SEQ ID NO: 24; the CDR-L2 of the IL-4 binding domain comprises the amino acid sequence of SEQ ID NO: 12, and the CDR-L3 of the IL-4 binding domain comprises the amino acid sequence of SEQ ID NO: 25, and
(iii) the IL-13 binding domain comprises a heavy chain variable region (IL13-VH) and a light chain variable region (IL13-VL), wherein the CDR-H1 of the IL-13 binding domain comprises the amino acid sequence of SEQ ID NO: 41; the CDR-H2 of the IL-13 binding domain comprises the amino acid sequence of SEQ ID NO: 42; the CDR-H3 of the IL-13 binding domain comprises the amino acid sequence of SEQ ID NO:-50; the CDR-L1 of the IL-13 binding domain comprises the amino acid sequence of SEQ ID NO: 53; the CDR-L2 of the IL-13 binding domain comprises the amino acid sequence of SEQ ID NO: 37, and the CDR-L3 of the IL-13 binding domain comprises the amino acid sequence of SEQ ID NO: 38.

E450. The antibody of E445-449, wherein
(i) the p40 binding domain comprises p40-VH of SEQ ID NO: 169, and a p40-VL of SEQ ID NO: 175;
(ii) the IL-4 binding domain comprises a IL4-VH of SEQ ID NO: 22, and a IL4-VL of SEQ ID NO: 26;
(iii) the IL-13 binding domain comprises a IL13-VH of SEQ ID NO: 51, and a IL13-VL of SEQ ID NO: 54.

E451. The antibody of E445-450, wherein
(i) the p40 binding domain comprises a p40-VH sequence encoded by the plasmid deposited at the ATCC and having ATCC Accession No. PTA-127206, and a p40-VL sequence encoded by the plasmid deposited at the ATCC and having ATCC Accession No. PTA-127205.
(ii) the IL-4 binding domain comprises IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) the IL-13 binding domain comprises an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

E452. The antibody of E445-E451, wherein the p40 binding domain is fused with or without a linker to the IL-13 binding domain.

E453. The antibody of E445-E451, wherein the p40 binding domain is fused with or without a linker to the IL-4 binding domain.

E454. The antibody of E445-E451, wherein the IL-13 binding domain is fused with or without a linker to the IL-4 binding domain.

E455. The antibody of any one of E452-E454, wherein the fusion is with a linker.

E456. The antibody of any one of E452-E455, wherein the IL-13 binding domain is fused with a linker to the IL-4 binding domain.

E457. The antibody of E456, wherein the linker comprises SEQ ID NO: 104.

E458. The antibody of any one of E445-E457, wherein the antibody comprises a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site.

E459. The antibody of E458, wherein the first and second polypeptide chains associate together to form an antibody comprising two arms; a dual Fab arm comprising the first Fab domain and the second Fab domain, and a single Fab arm comprising the third Fab domain.

E460. The antibody of E458-E459, wherein the firth polypeptide chain comprises the sequence EPKSC (SEQ ID NO: 122) at the C-terminus.

E461. The antibody of E458-E460, wherein the first antigen binding site specifically binds IL-13, the second antibody binding site specifically binds IL-4, and the third antigen binding site specifically binds p40.

E462. The antibody of E458-E461, wherein the first Fab domain, second Fab domain, and third Fab domain each comprise a different option selected from (i), (ii), and (iii) as follows:
(i) the p40-Fab of E166,
(ii) the IL4-Fab of E234, and
(iii) the IL13-Fab of E302.

E463. The antibody of E458-E462, wherein the first Fab domain is the IL13-Fab of E302, the second Fab domain is the IL4-Fab of E234, and the third Fab domain is the p40-Fab of E166.

E464. The antibody of E458-E463, wherein the first polypeptide comprises a first Fc chain, and the second polypeptide comprises a second Fc chain.

E465. The antibody of E464, wherein the first Fc chain and the second Fc chain each contain one or more amino acid modifications that promote the association of the first Fc chain with the second Fc chain.

E466. The antibody of E464-E465, wherein the first Fc chain comprises a first CH3 domain, and the second Fc chain comprises a second CH3 domain, and the first CH3 domain and the second CH3 domain each comprise a different and complementary sequence, and the different and complementary sequences are selected from one of the following pairs of different and complementary sequences:
(i) SEQ ID NO: 106 and SEQ ID NO:111;
(ii) SEQ ID NO:147 and SEQ ID NO: 148; and
(iii) SEQ ID NO: 124, and SEQ ID NO:127.

E467. The antibody of E466, wherein the first CH3 domain and the second CH3 domain comprise SEQ ID NO: 124 and SEQ ID NO: 127.

E468. The antibody of E458-E467, wherein the identity of the first, second, third, fourth, and fifth polypeptide chains is selected from the group consisting of
(i) the first polypeptide chain comprises SEQ ID NO: 186, the second polypeptide chain comprises SEQ ID NO: 130, the third polypeptide chain comprises SEQ ID NO: 176, the fourth polypeptide chain comprises SEQ ID NO: 27, and the fifth polypeptide chain comprises SEQ ID NO: 122;
(ii) the first polypeptide chain comprises SEQ ID NO: 146, the second polypeptide chain comprises SEQ ID NO: 178, the third polypeptide chain comprises SEQ ID NO: 109, the fourth polypeptide chain comprises SEQ ID NO: 196, and the fifth polypeptide chain comprises SEQ ID NO: 177;
(iii) the first polypeptide chain comprises SEQ ID NO: 112, the second polypeptide chain comprises SEQ ID NO: 179, the third polypeptide chain comprises SEQ ID NO: 196, the fourth polypeptide chain comprises SEQ ID NO: 109, and the fifth polypeptide chain comprises SEQ ID NO: 177;
(iv) the first polypeptide chain comprises SEQ ID NO: 181, the second polypeptide chain comprises SEQ ID NO 180, the third polypeptide chain comprises SEQ ID NO: 182, the fourth polypeptide chain comprises SEQ ID NO: 109, and the fifth polypeptide chain comprises SEQ ID NO: 122;
(v) the first polypeptide chain comprises SEQ ID NO: 118, the second polypeptide chain comprises SEQ ID NO: 183, the third polypeptide chain comprises SEQ ID NO: 120, the fourth polypeptide chain comprises SEQ ID NO: 116, and the fifth polypeptide chain comprises SEQ ID NO: 177;
(vi) the first polypeptide chain comprises SEQ ID NO: 185, the second polypeptide chain comprises SEQ ID NO: 125, the third polypeptide chain comprises SEQ ID NO: 176, the fourth polypeptide chain comprises SEQ ID NO: 207, and the fifth polypeptide chain comprises SEQ ID NO: 122; and
(vii) the first polypeptide chain comprises SEQ ID NO: 185, the second polypeptide chain comprises SEQ ID NO: 125, the third polypeptide chain comprises SEQ ID NO: 176, the fourth polypeptide chain comprises SEQ ID NO: 27, and the fifth polypeptide chain comprises SEQ ID NO: 122.

E469. The antibody of any one of E458-E468, wherein the first polypeptide chain comprises SEQ ID NO: 186, the second polypeptide chain comprises SEQ ID NO: 130, the third polypeptide chain comprises SEQ ID NO: 176, the fourth polypeptide chain comprises SEQ ID NO: 27, and the fifth polypeptide chain comprises SEQ ID NO: 122.

E470. An isolated antibody that specifically binds to p40, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a p40 binding site, and wherein the first polypeptide chain comprises SEQ ID NO: 186, the second polypeptide chain comprises SEQ ID NO: 130, the third polypeptide chain comprises SEQ ID NO: 176, the fourth polypeptide chain comprises SEQ ID NO: 27, and the fifth polypeptide chain comprises SEQ ID NO: 122.

E471. An isolated antibody that specifically binds p40, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a p40 binding site, and
wherein the first polypeptide chain comprises a sequence encoded by the plasmid deposited at the ATCC and having ATCC Accession No. PTA-127204; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127203; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

E472. The antibody of E445-E471, wherein the antibody has a viscosity of less than 20 cP at concentrations of at least 100 mg/mL in a buffer of 20 mM Histidine, 8.5% sucrose, 0.05 mg/mL EDTA pH 6.0.

E473. The antibody of E445-E472, wherein the antibody has a viscosity of less than 12 cP at concentrations of at least 50 mg/m in a buffer of 20 mM Histidine, 8.5% sucrose, 0.05 mg/mL EDTA pH 6.0 L.

E474. The antibody of E445-E473, wherein the antibody has a terminal half-life of at least 12 days in cynomolgus monkeys.

E475. The antibody of E445-E474, wherein the antibody has a terminal half-life of at least 18 days in TG-32 mice.

E476. The antibody of E445-E475, wherein the antibody binds human IL-4 with an affinity constant of less than 220 pM as measured by SPR.

E477. The antibody of E445-E476, wherein the antibody binds human IL-13 with an affinity constant of less than 220 pM as measured by SPR.

E478. The antibody of E445-E477, wherein the antibody binds human IL-12 with an affinity constant of less than 130 pM as measured by SPR.

E479. The antibody of E445-E478, wherein the antibody binds human IL-23 with an affinity constant of less than 100 pM as measured by SPR.

E480. The antibody of E445-E479, wherein the antibody binds to human IL-4 with a binding affinity of less than 1 pM, as measured by KinExA in a fixed antigen assay in PBS.

E481. The antibody of E445-E480, wherein the antibody binds to cynomolgus IL-13 with a binding affinity of less than 2 pM, as measured by KinExA in a fixed antigen assay in PBS.

E482. The antibody of E445-E481, wherein the antibody is characterized by an IC₅₀ of less than 12 pM as measured in a human monocyte assay for neutralization of IL-4 induction of CD23.

E483. The antibody of E445-E482, wherein the antibody is characterized by an IC₅₀ of less than 12 pM as measured in a human monocyte assay for neutralization of cynomolgus monkey IL-4 induction of CD23.

E484. The antibody of E445-E483, wherein the antibody is characterized by an IC₅₀ of less than 12 pM as measured in a human monocyte assay for neutralization of cynomolgus monkey IL-13 induction of CD23.

E485. The antibody of E445-E484, wherein the antibody is characterized by an IC₅₀ of less than 45 pM as measured in a human monocyte assay for neutralization of IL-13 induction of CD23.

E486. The antibody of E445-E485, wherein the antibody is characterized by an IC₅₀ of less than 600 pM in a human IL-12 neutralization Kit-225 assay in human peripheral blood monocytes.

E487. The antibody of E445-E486, wherein the antibody is characterized by an IC₅₀ of less than 2100 pM in a cynomolgus IL-23 Kit-225 neutralization assay in human peripheral blood monocytes.

E488. The antibody of E445-E487, wherein the antibody is characterized by an IC₅₀ of less than 400 pM in a human IL-12 neutralization assay in human whole blood.

E489. The antibody of E445-E488, wherein the antibody is characterized by an IC₅₀ of less than 10,000 pM in a cynomolgus IL-23 neutralization assay in human whole blood.

E490. The antibody of any one of E445-489, for use as a medicament.

E491. The antibody of E490, wherein the use is for the treatment of one or more selected from the group consisting of non-alcoholic steatohepatitis (NASH), psoriasis, psoriatic arthritis, atopic dermatitis, Crohn's disease, ulcerative colitis, asthma (severe), allergy, alopecia, idiopathic pulmonary fibrosis, systemic sclerosis, keloids, systemic lupus erythematosus, primary biliary cirrhosis, and hidradenitis suppurativa.

E492. The antibody of any one of E490-E491, wherein the use is for the treatment of one or more selected from the group consisting of non-alcoholic steatohepatitis (NASH), atopic dermatitis, asthma (severe), alopecia, idiopathic pulmonary fibrosis, and systemic sclerosis.

E483. The antibody of any one of E490-E482, wherein the use is for atopic dermatitis.

E484. The antibody of any one of E490-E483, wherein the use is for non-alcoholic steatohepatitis (NASH).

E495. A pharmaceutical composition comprising a therapeutically effective amount of the antibody of E445-E484 and a pharmaceutically acceptable carrier.

E496. A method of treating a medical condition, comprising administering to a subject in need thereof a therapeutically effective amount of the antibody of any one of E445-E494, or the pharmaceutical composition of E495.

E497. The method of E496, wherein the condition is selected from the group consisting of non-alcoholic steatohepatitis (NASH), psoriasis, psoriatic arthritis, atopic dermatitis, Crohn's disease, ulcerative colitis, asthma (severe), allergy, alopecia, idiopathic pulmonary fibrosis, systemic sclerosis, keloids, systemic lupus erythematosus, primary biliary cirrhosis, and hidradenitis suppurativa.

E498. The method of any one of E496-E497, comprising administering said antibody or pharmaceutical composition, subcutaneously.

E499. The method of any one of E496-E498, wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E500. An antibody comprising a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site.

E501. The antibody of E500, wherein the first and second polypeptide chains associate together to form an antibody comprising two arms; a dual Fab arm comprising the first Fab domain and the second Fab domain, and a single Fab arm comprising the third Fab domain.

E502. The antibody of E501, wherein the first Fab comprises a first antigen associated VH (VH-1), a first antigen associated VL (VL-1), a first antigen associated CL (CL-1), and a first antigen associated CH1 (CH1-1).

E503. The antibody of E504, wherein the C-terminus of the VH-1 is covalently fused by a peptide bond to the N-terminus of the CH1-1.

E504. The antibody of E502-E503, wherein the C-terminus of the VL-1 is covalently fused by a peptide bond to the N-terminus of the CL-1.

E505. The antibody of E501-E504, wherein the second Fab comprises a second antigen associated VH (VH-2), a second antigen associated VL (VL-2), a second antigen associated CL (CL-2), and a second antigen associated CH1 (CH1-2).

E506. The antibody of E505, wherein the C-terminus of the VH-2 is covalently fused by a peptide bond to the N-terminus of the CH1-2.

E507. The antibody of E505-E506, wherein the C-terminus of the VL-2 is covalently fused by a peptide bond to the N-terminus of the CL-2.

E508. The antibody of E501-E507, wherein the third Fab comprises a third antigen associated VH (VH-3), a first antigen associated VL (VL-3), a first antigen associated CL (CL-3), and a first antigen associated CH1 (CH1-3).

E509. The antibody of E508, wherein the C-terminus of the VH-3 is covalently fused by a peptide bond to the N-terminus of the CH1-3.

E510. The antibody of E508-E509, wherein the C-terminus of the VL-3 is covalently fused by a peptide bond to the N-terminus of the CL-3.

E511. The antibody of E500-510, wherein the second polypeptide comprises from N-terminus to C-terminus, (VL-1)-(CL-1)-(linker)-(VH-2)-(CH1-2)-(second hinge)-(second CH2)-(second CH3); the fifth polypeptide comprises from N-terminus to C-terminus, (VH1)-(CL-1); and the fourth polypeptide comprises (VL-2)-(CL-2).

E512. The antibody of E500-E511, wherein the first polypeptide comprises from N-terminus to C-terminus, (VH-3)-(CH1-3)-(first hinge)-(first CH2)-(first CH3); and the third polypeptide may comprise (VL-3)-(CL-3).

E513. The antibody of E508-E512, wherein one or of more of the CH1-1 domain, CH1-2 domain, and CH1-3 domain may comprise a sequence selected from the group consisting of SEQ ID NO: 6, SEQ ID NO: 105, and SEQ ID NO: 110.

E514. The antibody of E508-E513, wherein one or more of the CL-1 domain, CL-2 domain, and CL-3 domain comprises a sequence selected from the group consisting of SEQ ID NO: 16, SEQ ID NO: 95, SEQ ID NO: 108, and SEQ ID NO: 113.

E515. The antibody of E512-E514, wherein the first hinge region and the second hinge region comprise a pair of sequences according to SEQ ID NO: 129 and SEQ ID NO: 131.

E516. The antibody of E512-E515, wherein one or both of the first CH2 domain and the second CH2 domain comprises a sequence according to SEQ ID NO: 8.

E517. The antibody of E512-E516, wherein the first CH3 domain and the second CH3 domain each comprise a different and complementary sequence, and the different and complementary sequences are selected from one of the following pairs of different and complementary sequences:
(i) SEQ ID NO: 111 and SEQ ID NO: 106;
(ii) SEQ ID NO: 111 and SEQ ID NO: 114;
(iii) SEQ ID NO: 114 and SEQ ID NO: 117;
(iv) SEQ ID NO: 124 and SEQ ID NO: 127;
(v) SEQ ID NO: 139 and SEQ ID NO: 141; and
(vi) SEQ ID NO: 147 and SEQ ID NO: 148.

E518. The antibody of E512-E517, wherein
(i) the CL-1 comprises a sequence according to SEQ ID NO: 16, the linker comprises a sequence according to SEQ ID NO: 104, the CH1-2 comprises a sequence according to SEQ ID NO: 6, the second hinge comprises a sequence according to SEQ ID NO: 129, the second CH2 comprises a sequence according to SEQ ID NO: 8, and the second CH3 comprises a sequence according to SEQ ID NO: 124;
(ii) the CH1-1 comprises a sequence according to SEQ ID NO: 6; and
(iii) the CL-2 comprises a sequence according to SEQ ID NO: 16.

E519. The antibody of E508-518, wherein the CL-3 comprises a sequence according to a sequence selected from the group consisting of SEQ ID NO: 16, SEQ ID NO: 95, SEQ ID NO: 108, SEQ ID NO: 113.

E520. The antibody of E508-E519, wherein the CL-3 comprises a sequence according to according to SEQ ID NO: 95.

E521. The antibody of E508-E519, wherein the CL-3 comprises a sequence according to SEQ ID NO: 16.

E522. The antibody of E508-E521, wherein the CH1-3 comprises a sequence according to SEQ ID NO: 6.

E523. The antibody of E512-E522, wherein the first hinge comprises a sequence according to SEQ ID NO: 131.

E524. The antibody of E512-E523, wherein the first CH2 comprises a sequence according to SEQ ID NO: 8.

E525. The antibody of E512-E524, wherein the first CH3 comprises a sequence according to SEQ ID NO:127.

E526. An isolated antibody of any one of E500-E525 that specifically binds to IL-4, and specifically binds to IL-13, comprising an IL-4 binding domain and an IL-13 binding domain.

E527. An isolated antibody of any one of E1-E525 that specifically binds to IL-4, and specifically binds to IL-13, comprising an IL-4 binding domain and an IL-13 binding domain.

E528. An isolated antibody that specifically binds to IL-4, and specifically binds to IL-13, comprising an IL-4 binding domain and an IL-13 binding domain.

E529. The antibody of E526-E528, wherein the specific binding to IL-4 is through an antibody of any one of E199-E259.

E530. The antibody of any one of E526-E529, wherein the specific binding to IL-13 is through an antibody of any one of E265-E330.

E531. The antibody of any one of E526-E530, wherein the antibody comprises at least one additional antigen binding domain that binds to at least one different target to both of IL-4 and IL-13.

E532. The antibody of E531, wherein the at least one different target is selected from the group consisting of IL-33, TSLP, and p40, and wherein when the target is IL-33, may optionally further comprise the antibody of E1-E71, and wherein when the target is TSLP, may optionally further comprise the antibody of E77-E143, and wherein when the target is p40, may optionally further comprise the antibody of E144-E193.

E533. The antibody of E531, wherein the at least one different target is not IL-33.

E534. The antibody of E531, wherein the at least one different target is not TSLP.

E535. The antibody of E531, wherein the at least one different target is not p40.

E536. The antibody of any one of E526-E535, wherein
(i) the IL-4 binding domain comprises a heavy chain variable region (IL4-VH) and a light chain variable region (IL4-VL), wherein the CDR-H1 of the IL-4 binding domain comprises the amino acid sequence of SEQ ID NO: 18; the CDR-H2 of the IL-4 binding domain comprises the amino acid sequence of SEQ ID NO: 2; the CDR-H3 of the IL-4 binding domain comprises the amino acid sequence of SEQ ID NO: 3; the CDR-L1 of the IL-4 binding domain comprises the amino acid sequence of SEQ ID NO: 24; the CDR-L2 of the IL-4 binding domain comprises the amino acid sequence of SEQ ID NO: 12, and the CDR-L3 of the IL-4 binding domain comprises the amino acid sequence of SEQ ID NO: 25, and
(ii) the IL-13 binding domain comprises a heavy chain variable region (IL13-VH) and a light chain variable region (IL13-VL), wherein the CDR-H1 of the IL-13 binding domain comprises the amino acid sequence of SEQ ID NO: 41; the CDR-H2 of the IL-13 binding domain comprises the amino acid sequence of SEQ ID NO: 42; the CDR-H3 of the IL-13 binding domain comprises the amino acid sequence of SEQ ID NO:-50; the CDR-L1 of the IL-13 binding domain comprises the amino acid sequence of SEQ ID NO: 53; the CDR-L2 of the IL-13 binding domain comprises the amino acid sequence of SEQ ID NO: 37, and the CDR-L3 of the IL-13 binding domain comprises the amino acid sequence of SEQ ID NO: 38.

E537. The antibody of E526-E536, wherein
(i) the IL-4 binding domain comprises an IL4-VH of SEQ ID NO: 22, and an IL4-VL of SEQ ID NO: 26;
(ii) the IL-13 binding domain comprises an IL13-VH of SEQ ID NO: 51, and an IL13-VL of SEQ ID NO: 54;

E538. The antibody of E531-E537, wherein the additional antigen binding domain is fused with or without a linker to the IL-13 binding domain.

E539. The antibody of E531-E537, wherein the additional antigen binding domain is fused with or without a linker to the IL-4 binding domain.

E540. The antibody of E531-E537, wherein the IL-13 binding domain is fused with or without a linker to the IL-4 binding domain.

E541. The antibody of any one of E538-E540, wherein the fusion is with a linker.

E542. The antibody of any one of E540-E541, wherein the IL-13 binding domain is fused with a linker to the IL-4 binding domain.

E543. The antibody of E542, wherein the linker comprises SEQ ID NO: 104.

E544. The antibody of any one of E526-E543, wherein the antibody comprises a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site.

E545. The antibody of E544, wherein the first and second polypeptide chains associate together to form an antibody comprising two arms; a dual Fab arm comprising the first Fab domain and the second Fab domain, and a single Fab arm comprising the third Fab domain.

E546. The antibody of E526-E545, wherein
(i) the first antigen binding site specifically binds IL-13, the second antibody binding site specifically binds IL-4, and the third antigen binding site specifically binds the at least one additional target;
(ii) first antigen binding site specifically binds IL-4, the second antibody binding site specifically binds IL-13, and the third antigen binding site specifically binds to the at least one additional target;
(iii) the first antigen binding site specifically binds IL-4, the second antibody binding site specifically binds the at least one additional target, and the third antigen binding site specifically binds IL-13;
(iv) wherein the first antigen binding site specifically binds IL-13, the second antibody binding site specifically binds the at least one additional target, and the third antigen binding site specifically binds IL-4;
(v) the first antigen binding site specifically binds the at least one additional target, the second antibody binding site specifically binds IL-13, and the third antigen binding site specifically binds IL-4; or
(vi) the first antigen binding site specifically binds the at least one additional target, the second antibody binding site specifically binds IL-4, and the third antigen binding site specifically binds IL-13.

E547. The antibody of E544-E546, wherein the first Fab domain is the IL13-Fab of E302, the second Fab domain is the IL4-Fab of E234, and the third Fab domain is the additional target-Fab.

E548. The antibody of E44-E547, wherein the fifth polypeptide chain comprises the sequence EPKSC (SEQ ID NO: 122) at the C-terminus.

E549. The antibody of E544-E548, wherein the first polypeptide comprises a first Fc chain, and the second polypeptide comprises a second Fc chain.

E550. The antibody of E549, wherein the first Fc chain and the second Fc chain each contain one or more amino acid modifications that promote the association of the first Fc chain with the second Fc chain.

E551. The antibody of E544-E550, wherein the first Fc chain comprises a first CH3 domain, and the second Fc chain comprises a second CH3 domain, and the first CH3 domain and the second CH3 domain each comprise a different and complementary sequence, and the different and complementary sequences are selected from one of the following pairs of different and complementary sequences:
(i) SEQ ID NO: 111 and SEQ ID NO: 106;
(ii) SEQ ID NO: 111 and SEQ ID NO: 114;
(iii) SEQ ID NO: 114 and SEQ ID NO: 117;
(iv) SEQ ID NO: 124 and SEQ ID NO: 127;
(v) SEQ ID NO: 139 and SEQ ID NO: 141; and
(vi) SEQ ID NO: 147 and SEQ ID NO: 148.

E552. The antibody of E551, wherein the first CH3 domain and the second CH3 domain comprise SEQ ID NO: 124 and SEQ ID NO: 127.

E553. The antibody of E544-E552, wherein the identity of the second, fourth, and fifth polypeptide chains is selected from the group consisting of
(i) the second polypeptide chain comprises SEQ ID NO: 145, the fourth polypeptide chain comprises SEQ ID NO: 196, and the fifth polypeptide chain comprises SEQ ID NO: 103;
(ii) the second polypeptide chain comprises SEQ ID NO: 107, the fourth polypeptide chain comprises SEQ ID NO: 109, and the fifth polypeptide chain comprises SEQ ID NO: 103;
(iii) the second polypeptide chain comprises SEQ ID NO 115, the fourth polypeptide chain comprises SEQ ID NO: 116, and the fifth polypeptide chain comprises SEQ ID NO: 103;
(iv) the second polypeptide chain comprises SEQ ID NO: 121, the fourth polypeptide chain comprises SEQ ID NO: 109, and the fifth polypeptide chain comprises SEQ ID NO: 103;
(v) the second polypeptide chain comprises SEQ ID NO: 125, the fourth polypeptide chain comprises SEQ ID NO: 208, and the fifth polypeptide chain comprises SEQ ID NO: 122;
(vi) the second polypeptide chain comprises SEQ ID NO: 130, the fourth polypeptide chain comprises SEQ ID NO: 27, and the fifth polypeptide chain comprises SEQ ID NO: 122;
(vii) the second polypeptide chain comprises SEQ ID NO: 133, the fourth polypeptide chain comprises SEQ ID NO: 27, and the fifth polypeptide chain comprises SEQ ID NO: 122;
(viii) the second polypeptide chain comprises SEQ ID NO: 144, the fourth polypeptide chain comprises SEQ ID NO: 136, and the fifth polypeptide chain comprises SEQ ID NO: 143;
(ix) the second polypeptide chain comprises SEQ ID NO: 135, the fourth polypeptide chain comprises SEQ ID NO: 136, and the fifth polypeptide chain comprises SEQ ID NO: 122;
(x) the second polypeptide chain comprises SEQ ID NO: 140, the fourth polypeptide chain comprises SEQ ID NO: 27, and the fifth polypeptide chain comprises SEQ ID NO: 122;
(xi) the second polypeptide chain comprises SEQ ID NO: 149, the fourth polypeptide chain comprises SEQ ID NO: 196, and the fifth polypeptide chain comprises SEQ ID NO: 150;
(xii) the second polypeptide chain comprises SEQ ID NO: 151, the fourth polypeptide chain comprises SEQ ID NO: 109, and the fifth polypeptide chain comprises SEQ ID NO: 150;
(xiii) the second polypeptide chain comprises SEQ ID NO 159, the fourth polypeptide chain comprises SEQ ID NO: 109, and the fifth polypeptide chain comprises SEQ ID NO: 150;
(xiv) the second polypeptide chain comprises SEQ ID NO: 162, the fourth polypeptide chain comprises SEQ ID NO: 197, and the fifth polypeptide chain comprises SEQ ID NO: 163;
(xv) the second polypeptide chain comprises SEQ ID NO: 154, the fourth polypeptide chain comprises SEQ ID NO: 196, and the fifth polypeptide chain comprises SEQ ID NO: 155;
(xvi) the second polypeptide chain comprises SEQ ID NO: 156, the fourth polypeptide chain comprises SEQ ID NO: 109, and the fifth polypeptide chain comprises SEQ ID NO: 155;
(xvii) the second polypeptide chain comprises SEQ ID NO: 152, the fourth polypeptide chain comprises SEQ ID NO: 196, and the fifth polypeptide chain comprises SEQ ID NO: 98;
(xviii) the second polypeptide chain comprises SEQ ID NO: 153, the fourth polypeptide chain comprises SEQ ID NO: 109, and the fifth polypeptide chain comprises SEQ ID NO: 98;
(xix) the second polypeptide chain comprises SEQ ID NO: 157, the fourth polypeptide chain comprises SEQ ID NO: 109, and the fifth polypeptide chain comprises SEQ ID NO: 158;
(xx) the second polypeptide chain comprises SEQ ID NO: 160, the fourth polypeptide chain comprises SEQ ID NO: 196, and the fifth polypeptide chain comprises SEQ ID NO: 158;
(xxi) the second polypeptide chain comprises SEQ ID NO: 164, the fourth polypeptide chain comprises SEQ ID NO: 197, and the fifth polypeptide chain comprises SEQ ID NO: 122; and
(xxii) the second polypeptide chain comprises SEQ ID NO: 178, the fourth polypeptide chain comprises SEQ ID NO: 196, and the fifth polypeptide chain comprises SEQ ID NO: 177;
(xxiii) the second polypeptide chain comprises SEQ ID NO: 179, the fourth polypeptide chain comprises SEQ ID NO: 109, and the fifth polypeptide chain comprises SEQ ID NO: 177;
(xxiv) the second polypeptide chain comprises SEQ ID NO 180, the fourth polypeptide chain comprises SEQ ID NO: 109, and the fifth polypeptide chain comprises SEQ ID NO: 122;
(xxv) the second polypeptide chain comprises SEQ ID NO: 183, the fourth polypeptide chain comprises SEQ ID NO: 116, and the fifth polypeptide chain comprises SEQ ID NO: 177;
(xxvi) the second polypeptide chain comprises SEQ ID NO: 125, the fourth polypeptide chain comprises SEQ ID NO: 207, and the fifth polypeptide chain comprises SEQ ID NO: 122;
(xxvii) the second polypeptide chain comprises SEQ ID NO: 125, the fourth polypeptide chain comprises SEQ ID NO: 27, and the fifth polypeptide chain comprises SEQ ID NO: 122; and E554. The antibody of any one of E544-E553, wherein the second polypeptide chain comprises SEQ ID NO: 130, the fourth polypeptide chain comprises SEQ ID NO: 27, and the fifth polypeptide chain comprises SEQ ID NO: 122.

E555. An isolated antibody that specifically binds to IL-4, and that specifically binds to IL-13, and at least one additional target, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an at least one additional target binding site, and
wherein the second polypeptide chain comprises SEQ ID NO: 130, the fourth polypeptide chain comprises SEQ ID NO: 27, and the fifth polypeptide chain comprises SEQ ID NO: 122.

E556. An antibody comprising an antibody Fc domain comprising a first Fc chain and a second Fc chain, wherein the first Fc chain and the second Fc chain each contain two amino acid modifications that promote the association of the first Fc chain with the second Fc chain, characterized in that
(i) the first Fc chain comprises D(H232)R and K(H440)R, and the second Fc chain comprises D(H232)E and L(H391)E; or
(ii) the first Fc chain comprises D(H232)E and K(H440)R, and the second Fc chain comprises L(H391)R and D(H232)E.

E557. The antibody of E556, wherein first Fc chain comprises, in N-terminal to C-terminal order, a first hinge region connected to a first CH2 region which is connected to a first CH3 region, and herein the second Fc chain comprises, in N-terminal to C-terminal order, a second hinge region connected to a second CH2 region which is connected to a second CH3 region, and wherein the first hinge region and second hinge region comprise a pair of sequences according to SEQ ID NO: 129 and SEQ ID NO: 131, and the first CH3 region and the second CH3 region comprise either of the following two pairs pair of sequences: SEQ ID NO: 124 and SEQ ID NO: 127; or SEQ ID NO: 147 and SEQ ID NO: 148.

E558. The antibody of any one of E556-E557, further comprising the antibody of one or more of E1-E71, E77-E143, E144-E193, E199-E259, E265-E330, E336-E381, E387-E435, E445-E484, and E500-E557.

E559. An isolated antibody comprising the CDRs of an antibody selected from one or more of Tables 80, 81, 82, 83, 84, 85, 86, and 87.

E560. An isolated antibody comprising the VH and VL of an antibody selected from one or more of Tables 80, 81, 82, 83, 84, 85, 86, and 87.

E561. An isolated antibody selected from one or more of Tables 80, 81, 82, 83, 84, 85, 86, and 87.

E562. An isolated polynucleotide, comprising one or more nucleotide sequences encoding the antibody of any one of E1-E71, E77-E143, E144-193, E199-259, E265-E330, E336-E381, E387-E435, E445-E484, and E500-E561.

E563. The polynucleotide of E562, wherein said polynucleotide is RNA.

E564. The polynucleotide of E562-E563, wherein said polynucleotide comprises at least one chemical modification.

E565. The polynucleotide of E564, wherein the chemical modification wherein is selected from pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine and 2′-O-methyl uridine.

E566. The polynucleotide of E562-E563, wherein said polynucleotide does not comprise a chemical modification.

E567. An isolated polynucleotide encoding the VH, VL, or both, of an antibody that binds IL-33, wherein the nucleic acid comprises: the nucleic acid sequence of SEQ ID NO: 202, the nucleic acid sequence of SEQ ID NO: 203, or both.

E568. An isolated polynucleotide encoding a VH bearing polypeptide and a VL bearing polypeptide, or both, of an antibody that binds to IL-33, wherein said nucleic acid comprises the nucleic acid sequence of SEQ ID NO: 190, the nucleic acid sequence of SEQ ID NO: 191, or both.

E569. An isolated polynucleotide encoding the VH, VL, or both, of an antibody that binds to IL-33, wherein said nucleic acid comprises the nucleic acid sequence of the insert of the plasmid deposited with the ATCC and having the Accession Number PTA-127209 the nucleic acid sequence of the insert of the plasmid deposited with the ATCC and having the Accession Number PTA-127210 or both.

E570. An isolated polynucleotide encoding a VH bearing polypeptide and a VL bearing polypeptide, or both, of an antibody that binds to IL-33, wherein said nucleic acid comprises the nucleic acid sequence of the insert of the plasmid deposited with the ATCC and having the Accession Number PTA-127207, the nucleic acid sequence of the insert of the plasmid deposited with the ATCC and having the Accession Number PTA-127208, or both.

E571. An isolated polynucleotide encoding the VH, VL, or both, of an antibody that binds TSLP, wherein the nucleic acid comprises: the nucleic acid sequence of SEQ ID NO: 204, the nucleic acid sequence of SEQ ID NO: 205, or both.

E572. An isolated polynucleotide encoding a VH bearing polypeptide and a VL bearing polypeptide, or both, of an antibody that binds to TSLP, wherein said nucleic acid comprises the nucleic acid sequence of SEQ ID NO: 192, the nucleic acid sequence of SEQ ID NO: 193, or both.

E573. An isolated polynucleotide encoding the VH, VL, or both, of an antibody that binds to TSLP, wherein said nucleic acid comprises the nucleic acid sequence of the insert of the plasmid deposited with the ATCC and having the Accession Number PTA-127200 the nucleic acid sequence of the insert of the plasmid deposited with the ATCC and having the Accession Number PTA-127199 or both.

E574. An isolated polynucleotide encoding a VH bearing polypeptide and a VL bearing polypeptide, or both, of an antibody that binds to TSLP, wherein said nucleic acid comprises the nucleic acid sequence of the insert of the plasmid deposited with the ATCC and having the Accession Number PTA-127202, the nucleic acid sequence of the insert of the plasmid deposited with the ATCC and having the Accession Number PTA-127201, or both.

E575. An isolated polynucleotide encoding a VH bearing polypeptide and a VL bearing polypeptide, or both, of an antibody that binds to p40, wherein said nucleic acid comprises the nucleic acid sequence of SEQ ID NO: 194, the nucleic acid sequence of SEQ ID NO: 195, or both.

E576. An isolated polynucleotide encoding a VH bearing polypeptide and a VL bearing polypeptide, or both, of an antibody that binds to p40, wherein said nucleic acid comprises the nucleic acid sequence of the insert of the plasmid deposited with the ATCC and having the Accession Number PTA-127204, the nucleic acid sequence of the insert of the plasmid deposited with the ATCC and having the Accession Number PTA-127203, or both.

E577. An isolated polynucleotide encoding the VH, VL, or both, of an antibody that binds IL-4, wherein the nucleic acid comprises: the nucleic acid sequence of SEQ ID NO: 200, the nucleic acid sequence of SEQ ID NO: 201, or both.

E578. An isolated polynucleotide encoding a VH bearing polypeptide and a VL bearing polypeptide, or both, of an antibody that binds to IL-4, wherein said nucleic acid comprises the nucleic acid sequence of SEQ ID NO: 188, the nucleic acid sequence of SEQ ID NO: 189, or both.

E579. An isolated polynucleotide encoding the VH, VL, or both, of an antibody that binds to IL-4, wherein said nucleic acid comprises the nucleic acid sequence of the insert of the plasmid deposited with the ATCC and having the Accession Number PTA-127198 the nucleic acid sequence of the insert of the plasmid deposited with the ATCC and having the Accession Number PTA-127197 or both.

E580. An isolated polynucleotide encoding a VH bearing polypeptide and a VL bearing polypeptide, or both, of an antibody that binds to IL-4, wherein said nucleic acid comprises the nucleic acid sequence of the insert of the plasmid deposited with the ATCC and having the Accession Number PTA-127192, the nucleic acid sequence of the insert of the plasmid deposited with the ATCC and having the Accession Number PTA-127194, or both.

E581. An isolated polynucleotide encoding the VH, VL, or both, of an antibody that binds IL-13, wherein the nucleic acid comprises: the nucleic acid sequence of SEQ ID NO: 196, the nucleic acid sequence of SEQ ID NO: 195, or both.

E582. An isolated polynucleotide encoding a VH bearing polypeptide and a VL bearing polypeptide, or both, of an antibody that binds to IL-13, wherein said nucleic acid comprises the nucleic acid sequence of SEQ ID NO: 187, the nucleic acid sequence of SEQ ID NO: 188, or both.

E583. An isolated polynucleotide encoding the VH, VL, or both, of an antibody that binds to IL-13, wherein said nucleic acid comprises the nucleic acid sequence of the insert of the plasmid deposited with the ATCC and having the Accession Number PTA-127196 the nucleic acid sequence of the insert of the plasmid deposited with the ATCC and having the Accession Number PTA-127195 or both.

E584. An isolated polynucleotide encoding a VH bearing polypeptide and a VL bearing polypeptide, or both, of an antibody that binds to IL-13, wherein said nucleic acid comprises the nucleic acid sequence of the insert of the plasmid deposited with the ATCC and having the Accession Number PTA-127193, the nucleic acid sequence of the insert of the plasmid deposited with the ATCC and having the Accession Number PTA-127192, or both.

E585. An isolated polynucleotide encoding one or more of the first, second, third, fourth, or fifth polypeptides of an anti-IL-4/IL-13/IL-33 antibody, comprising
(i) an IL33-VH sequence encoded by the plasmid deposited at the ATCC and having ATCC Accession No. PTA-127210, and an IL33-VL sequence encoded by the plasmid deposited at the ATCC and having ATCC Accession No. PTA-127209;
(ii) an IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

E586. An isolated polynucleotide encoding one or more of the first, second, third, fourth, or fifth polypeptides of an anti-IL-4/IL-13/IL-33 antibody, wherein the isolated antibody specifically binds IL-33, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an IL-33 binding site, and
wherein the first polypeptide chain comprises a sequence encoded by the plasmid deposited at the ATCC and having ATCC Accession No. PTA-127208; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127207; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

E587. An isolated polynucleotide encoding one or more of the first, second, third, fourth, or fifth polypeptides of an anti-IL-4/IL-13/TSLP antibody, wherein the antibody comprises
(i) a TSLP-VH sequence encoded by the plasmid deposited at the ATCC and having ATCC Accession No. PTA-127200, and a TSLP-VL sequence encoded by the plasmid deposited at the ATCC and having ATCC Accession No. PTA0-127199.
(ii) a IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

E588. An isolated polynucleotide encoding one or more of the first, second, third, fourth, or fifth polypeptides of an anti-IL-4/IL-13/TSLP antibody, wherein the antibody comprises a first, second, third, fourth, and fifth polypeptide chain and wherein
(i) the second and fifth polypeptide chains together form a first Fab domain comprising a IL-13 binding site;
(ii) the second and fourth polypeptide chains together form a second Fab domain comprising an IL-4 binding site;
(iii) the first and third polypeptide chains together form a third Fab domain comprising a TSLP binding site, and
wherein the first polypeptide chain comprises a sequence encoded by the plasmid deposited at the ATCC and having ATCC Accession No. PTA-127202; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127201; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

E589. An isolated polynucleotide encoding one or more of the first, second, third, fourth, or fifth polypeptides of an anti-IL-4/IL-13/p40 antibody, wherein the antibody comprises
(i) a p40-VH sequence encoded by the plasmid deposited at the ATCC and having ATCC Accession No. PTA-127206, and a p40-VL sequence encoded by the plasmid deposited at the ATCC and having ATCC Accession No. PTA-127205.
(ii) an IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

E590. An isolated polynucleotide encoding one or more of the first, second, third, fourth, or fifth polypeptides of an anti-IL-4/IL-13/p40 antibody, wherein the antibody comprises a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a p40 binding site, and
wherein the first polypeptide chain comprises a sequence encoded by the plasmid deposited at the ATCC and having ATCC Accession No. PTA-127204; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127203; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

E591. A vector comprising the polynucleotide of E562-E590.

E592. An isolated host cell comprising the polynucleotide of E562-E590, or the vector of E591.

E593. A method of producing an isolated antibody, comprising culturing the host cell of E592 under conditions that result in production of the antibody, and recovering the antibody.

E594. A pharmaceutical composition comprising a therapeutically effective amount of the antibody of any one of E1-E71, E77-E143, E144-193, E199-259, E265-E330, E336-E381, E387-E435, E445-E484, and E500-E557 and a pharmaceutically acceptable carrier.

E595. A method for generating a heterotrimeric antibody comprising a dual Fab arm and a single Fab arm, wherein the dual Fab arm comprises a first Fab domain connected to a second Fab domain which is connected to a first Fc domain, and the single Fab arm comprises a third Fab domain connected to a second Fc domain, and the method comprising
(i) a first preassembly step wherein the dual Fab arm is incubated in a first preassembly conditioning buffer at a temperature of between 2-10° C. and wherein the pH of the first preassembly conditioning buffer is 1-3 units below the isoelectric point (pI) of the dual Fab arm; and
(ii) a second preassembly step wherein the single Fab arm is incubated in a second preassembly conditioning buffer at a temperature of between 2-10° C. and wherein the pH of the second preassembly conditioning buffer is 1-3 units below the isoelectric point (pI) of the single Fab arm; and
(iii) a third assembly step, wherein the dual Fab arm and single Fab arm from step (i) and (ii) are mixed together in an assembly buffer for between 1-24 hours

E596. An isolated antibody that specifically binds TSLP, comprising the CDRs of an antibody selected from one or more of Tables 83, 84, and 87.

E597. An isolated antibody that specifically binds TSLP, comprising the VH and VL of an antibody selected from one or more of Tables 83, 84, and 87.

E598. An isolated antibody that specifically binds TSLP, selected from one or more of Tables 83, 84, and 87.

E599. The antibody of any one of E381-E384, wherein
(i) the TSLP binding domain comprises a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 211; and
(ii) the IL-4 binding domain comprises a heavy chain variable region (IL4-VH) and a light chain variable region (IL4-VL), wherein the CDR-H1 comprises the amino acid sequence of SEQ ID NO: 18; the CDR-H2 comprises the amino acid sequence of SEQ ID NO: 2; the CDR-H3 comprises the amino acid sequence of SEQ ID NO: 3; the CDR-L1 comprises the amino acid sequence of SEQ ID NO: 24; the CDR-L2 comprises the amino acid sequence of SEQ ID NO: 12, and the CDR-L3 comprises the amino acid sequence of SEQ ID NO: 25, and
(iii) the IL-13 binding domain comprises a heavy chain variable region (IL13-VH) and a light chain variable region (IL13-VL), wherein the CDR-H1 comprises the amino acid sequence of SEQ ID NO: 41; the CDR-H2 comprises the amino acid sequence of SEQ ID NO: 42; the CDR-H3 comprises the amino acid sequence of SEQ ID NO:-50; the CDR-L1 comprises the amino acid sequence of SEQ ID NO: 53; the CDR-L2 comprises the amino acid sequence of SEQ ID NO: 37, and the CDR-L3 comprises the amino acid sequence of SEQ ID NO: 38.

E600. The antibody of E387-E390, or E599, wherein
(i) the TSLP binding portion comprises the TSLP-VH of SEQ ID NO: 92, and the TSLP-VL of SEQ ID NO: 213;
(ii) the IL-4 binding portion comprises a IL4-VH of SEQ ID NO: 22, and a IL4-VL of SEQ ID NO: 26; and
(iii) the IL-13 binding portion comprises the IL13-VH of SEQ ID NO: 51, and the IL13-VL of SEQ ID NO: 54.

E601. The antibody of E387-E390, or E599-E600, wherein
(i) the TSLP binding domain comprises a TSLP-VH sequence encoded by the plasmid deposited at the ATCC and having ATCC Accession No. PTA-127200, and a TSLP-VL sequence encoded by the plasmid deposited at the ATCC and having ATCC Accession No. PTA-______.
(ii) the IL-4 binding domain comprises IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) the IL-13 binding domain comprises an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

E602. An isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an TSLP binding site; and
wherein the first polypeptide chain comprises SEQ ID NO: 165, the second polypeptide chain comprises SEQ ID NO: 130, the third polypeptide chain comprises SEQ ID NO: 215, the fourth polypeptide chain comprises SEQ ID NO: 27, and the fifth polypeptide chain comprises SEQ ID NO: 122.

E603. An isolated antibody that specifically binds TSLP, that specifically binds to IL-4, and that specifically binds to IL-13, comprising a first, second, third, fourth, and fifth polypeptide chain and wherein
(iv) the second and fifth polypeptide chains together form a first Fab domain comprising an IL-13 binding site;
(v) the second and fourth polypeptide chains together form a second Fab domain comprising an IL-4 binding site;
(vi) the first and third polypeptide chains together form a third Fab domain comprising a TSLP binding site, and
wherein the first polypeptide chain comprises a sequence encoded by the plasmid deposited at the ATCC and having ATCC Accession No. PTA-127202; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-______; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

E604. The antibody of any one of E381-E390, wherein
(i) the TSLP binding domain comprises a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 212; and
(ii) the IL-4 binding domain comprises a heavy chain variable region (IL4-VH) and a light chain variable region (IL4-VL), wherein the CDR-H1 comprises the amino acid sequence of SEQ ID NO: 18; the CDR-H2 comprises the amino acid sequence of SEQ ID NO: 2; the CDR-H3 comprises the amino acid sequence of SEQ ID NO: 3; the CDR-L1 comprises the amino acid sequence of SEQ ID NO: 24; the CDR-L2 comprises the amino acid sequence of SEQ ID NO: 12, and the CDR-L3 comprises the amino acid sequence of SEQ ID NO: 25, and
(iii) the IL-13 binding domain comprises a heavy chain variable region (IL13-VH) and a light chain variable region (IL13-VL), wherein the CDR-H1 comprises the amino acid sequence of SEQ ID NO: 41; the CDR-H2 comprises the amino acid sequence of SEQ ID NO: 42; the CDR-H3 comprises the amino acid sequence of SEQ ID NO:-50; the CDR-L1 comprises the amino acid sequence of SEQ ID NO: 53; the CDR-L2 comprises the amino acid sequence of SEQ ID NO: 37, and the CDR-L3 comprises the amino acid sequence of SEQ ID NO: 38.

E605. The antibody of E387-E390, or E604, wherein
(i) the TSLP binding portion comprises the TSLP-VH of SEQ ID NO: 92, and the TSLP-VL of SEQ ID NO: 214;
(ii) the IL-4 binding portion comprises a IL4-VH of SEQ ID NO: 22, and a IL4-VL of SEQ ID NO: 26; and
(iii) the IL-13 binding portion comprises the IL13-VH of SEQ ID NO: 51, and the IL13-VL of SEQ ID NO: 54.

E606. The antibody of E387-E390, or E602-E603, wherein
(i) the TSLP binding domain comprises a TSLP-VH sequence encoded by the plasmid deposited at the ATCC and having ATCC Accession No. PTA-127200, and a TSLP-VL sequence encoded by the plasmid deposited at the ATCC and having ATCC Accession No. PTA0-______.
(ii) the IL-4 binding domain comprises IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) the IL-13 binding domain comprises an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

E607. An isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an TSLP binding site; and
wherein the first polypeptide chain comprises SEQ ID NO: 165, the second polypeptide chain comprises SEQ ID NO: 130, the third polypeptide chain comprises SEQ ID NO: 216, the fourth polypeptide chain comprises SEQ ID NO: 27, and the fifth polypeptide chain comprises SEQ ID NO: 122.

E608. An isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a TSLP binding site, and
wherein the first polypeptide chain comprises a sequence encoded by the plasmid deposited at the ATCC and having ATCC Accession No. PTA-127202; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-______; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

E609. The antibody of any one of E77-E143, or E596-E608, for use as a medicament.

E610. The antibody of E609, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis.

E611. The antibody of any one of E147-E148, or E596-E610, wherein the use is for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, and non-alcoholic steatohepatitis (NASH)

E612. The antibody of any one of E147-E149, or E596-E611, wherein the use is for atopic dermatitis.

E613. The antibody of any one of E147-E149, or E596-E612, wherein the use is for non-alcoholic steatohepatitis (NASH).

E614. A pharmaceutical composition comprising a therapeutically effective amount of the antibody of E77-E143 or E596-E608, and a pharmaceutically acceptable carrier.

E615. A method of treating a medical condition, comprising administering to a subject in need thereof a therapeutically effective amount of the antibody of any one of E77-E143, or E596-E608, or the pharmaceutical composition of E614.

E616. The method of E615, wherein the condition is selected from the group consisting of atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis.

E617. The method of any one of E615-E616, comprising administering said antibody or pharmaceutical composition, subcutaneously.

E618. The method of any one of E615-E617, wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E619. The antibody of any one of E1-E71, E77-E143, E144-193, E199-259, E265-E330, E336-E381, E387-E435, E445-E484, E500-E557, or E596-E612, for use in the inhibition of tumor growth.

E620. The antibody of any one of E1-E71, E77-E143, E144-193, E199-259, E265-E330, E336-E381, E387-E435, E445-E484, E500-E557, E596-E612, or E619, for use in the inhibition of progression of malignant cell growth in a patient.

E621. The antibody of any one of E1-E71, E77-E143, E144-193, E199-259, E265-E330, E336-E381, E387-E435, E445-E484, E500-E557, E596-E612, or E619-E620, for use in the inhibition of metastasis of malignant cells in a patient.

E622. The antibody of any one of E1-E71, E77-E143, E144-193, E199-259, E265-E330, E336-E381, E387-E435, E445-E484, E500-E557, E596-E612, or E619-E621, for use in the induction of tumor regression in a patient.

E623. The antibody of any one of E1-E71, E77-E143, E144-193, E199-259, E265-E330, E336-E381, E387-E435, E445-E484, E500-E557, E596-E612, or E619-E622, for use in the treatment of a cancer presenting with a solid tumor.

E624. The antibody of any one of E1-E71, E77-E143, E144-193, E199-259, E265-E330, E336-E381, E387-E435, E445-E484, E500-E557, E596-E612, or E619-E623, wherein the use is for the treatment of one or more selected from the group consisting of bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

E625. The antibody of any one of E1-E71, E77-E143, E144-193, E199-259, E265-E330, E336-E381, E387-E435, E445-E484, E500-E557, E596-E612, or E619-E624, wherein the use is for the treatment of one or more selected from the group consisting of renal cell carcinoma (RCC), bladder cancer, breast cancer, clear cell kidney cancer, head/neck squamous cell carcinoma (SCCHN), lung squamous cell carcinoma, malignant melanoma, non-small-cell lung cancer (NSCLC), ovarian cancer, pancreatic cancer, prostate cancer, small-cell lung cancer (SCLC) or triple negative breast cancer.

E626. The antibody of any one of E1-E71, E77-E143, E144-193, E199-259, E265-E330, E336-E381, E387-E435, E445-E484, E500-E557, E596-E612, or E619-E625, wherein the use is for the treatment of one or more selected from the group consisting of a Heme malignancy and in some embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

E627. A method for treating a cancer in a subject comprising administering to the subject a combination therapy which comprises a first anti-cancer therapeutic agent and a second anti-cancer therapeutic agent, wherein the first anti-cancer therapeutic agent is the antibody against one or more of IL-4, IL-13, and TSLP, and wherein the second anti-cancer therapeutic agent is selected from the group consisting of an anti-OX40 antibody, an anti-4-1 BB antibody, an anti-HER2 antibody, a PD-1 pathway antagonist, an anti-PD-1 antibody, an anti-PD-L1 antibody, a TLR3 agonist, a TLR 7/8 agonist, a TLR9 agonist, a bispecific anti-CD47/anti-PD-L1 antibody, and a bispecific anti-P-cadherin/anti-CD3 antibody.

E628. The method of E627, wherein the first anti-cancer therapeutic agent comprises an anti-IL-4 antibody.

E629. The method of E627-E628, wherein the first anti-cancer therapeutic agent comprises an anti-IL-4 antibody of any one of E199-E259

E630. The method of E627-E631, wherein the first anti-cancer therapeutic agent comprises an anti-IL-13 antibody.

E631. The method of E627-E630, wherein the first anti-cancer therapeutic agent comprises an anti-IL-13 antibody of any one of E265-E330.

E632. The method of E627-E631, wherein the first anti-cancer therapeutic agent comprises an anti-TSLP antibody.

E633. The method of E627-E632, wherein the first anti-cancer therapeutic agent comprises an anti-TSLP antibody of any one of E77-E143, or E596-E598.

E634. The method of E627-E633, wherein the first anti-cancer therapeutic agent comprises an IL-4/IL-13 antibody.

E635. The method of E627-E634, the first anti-cancer therapeutic agent comprises an IL-4/IL-13 antibody, and wherein the IL-4/IL-13 antibody comprises the IL-4/IL-13 antibody of any one of E526-E558.

E636. The method of E627-E635, wherein the first anti-cancer therapeutic agent comprises an IL-4/IL-13/TSLP antibody.

E637. The method of E627-E636, wherein the first anti-cancer therapeutic agent comprises an IL-4/IL-13/TSLP antibody and wherein the IL-4/IL-13/TSLP antibody comprises the antibody of any one of E387-E435, or E599-E613.

E638. The method of E627-E637, wherein the first anti-cancer therapeutic agent comprises an IL-4/IL-13/TSLP antibody and wherein the IL-4/IL-13/TSLP antibody comprises the antibody of any one of E412.

E639. The method of E627-E637, wherein the first anti-cancer therapeutic agent comprises an IL-4/IL-13/TSLP antibody and wherein the IL-4/IL-13/TSLP antibody comprises the antibody of any one of E603.

E640. The method of E627-E637, wherein the first anti-cancer therapeutic agent comprises an IL-4/IL-13/TSLP antibody and wherein the IL-4/IL-13/TSLP antibody comprises the antibody of any one of E607.

E641. The method of E627-E640, wherein the second anti-cancer therapeutic agent is a PD-1 pathway antagonist.

E642. The method of E627-E641, wherein the second anti-cancer therapeutic agent is a PD-1 antagonist.

E643. The method of E627-E642, wherein the second anti-cancer therapeutic agent is a PD-1 antagonist, and the PD-1 antagonist is selected from the group consisting of sasanlimab, BCD-100, camrelizumab, cemiplimab, genolimzumab, MEDI0680, nivolumab, pembrolizumab, sintilimab, spartalizumab, STI-A1110, tislelizumab, atezolizumab, durvalumab, BMS-936559 (MDX-1105), LY3300054, TSR-042.

E644. The method of E627-E643, wherein the second anti-cancer therapeutic agent is a PD-1 antagonist, and the PD-1 antagonist is an antibody that comprises a VH as shown in SEQ ID NO: 4 and a VL as shown in SEQ ID NO: 8 of U.S. Ser. No. 10/155,037.

E644. The method of E627-E643, wherein the second anti-cancer therapeutic agent is a PD-1 antagonist, and the PD-1 antagonist is sasanlimab.

E645. The method of E627-E646, wherein the second anti-cancer therapeutic agent is a PD-1 antagonist, and the PD-1 antagonist is sasanlimab, and is an antibody comprising a HC comprising a sequence according to SEQ ID NO; 225 and a light chain comprising a sequence according to SEQ ID NO: 226.

E646. A method for treating a cancer in a subject comprising administering to the subject a combination therapy which comprises a first anti-cancer therapeutic agent and a second anti-cancer therapeutic agent, wherein the first anti-cancer therapeutic agent is the IL-4/IL-13/TSLP antibody of E412, and the second anti-cancer therapeutic is a PD-1 antagonist antibody comprising a HC comprising a sequence according to SEQ ID NO; 225 and a light chain comprising a sequence according to SEQ ID NO: 226.

E647. A method for treating a cancer in a subject comprising administering to the subject a combination therapy which comprises a first anti-cancer therapeutic agent and a second anti-cancer therapeutic agent, wherein the first anti-cancer therapeutic agent is the IL-4/IL-13/TSLP antibody of E603, and the second anti-cancer therapeutic is a PD-1 antagonist antibody comprising a HC comprising a sequence according to SEQ ID NO; 225 and a light chain comprising a sequence according to SEQ ID NO: 226.

E648. A method for treating a cancer in a subject comprising administering to the subject a combination therapy which comprises a first anti-cancer therapeutic agent and a second anti-cancer therapeutic agent, wherein the first anti-cancer therapeutic agent is the IL-4/IL-13/TSLP antibody of E607 and the second anti-cancer therapeutic is a PD-1 antagonist antibody comprising a HC comprising a sequence according to SEQ ID NO; 225 and a light chain comprising a sequence according to SEQ ID NO: 226.

E649. A medicament comprising a first anti-cancer agent, and a second anti-cancer agent, wherein the first anti-cancer therapeutic agent is the IL-4/IL-13/TSLP antibody of E412 and the second anti-cancer therapeutic is a PD-1 antagonist antibody comprising a HC comprising a sequence according to SEQ ID NO; 225 and a light chain comprising a sequence according to SEQ ID NO: 226.

E649. A medicament comprising a first anti-cancer agent, and a second anti-cancer agent, wherein the first anti-cancer therapeutic agent is the IL-4/IL-13/TSLP antibody of E603 and the second anti-cancer therapeutic is a PD-1 antagonist antibody comprising a HC comprising a sequence according to SEQ ID NO; 225 and a light chain comprising a sequence according to SEQ ID NO: 226.

E649. A medicament comprising a first anti-cancer agent, and a second anti-cancer agent, wherein the first anti-cancer therapeutic agent is the IL-4/IL-13/TSLP antibody of E607 and the second anti-cancer therapeutic is a PD-1 antagonist antibody comprising a HC comprising a sequence according to SEQ ID NO; 225 and a light chain comprising a sequence according to SEQ ID NO: 226.

E650. The method or medicament as set forth in any one of E627-E649, wherein the cancer presents with a solid tumor.

E651. The method or medicament as set forth in any one of E627-E650, wherein the cancer is one or more selected from the group consisting of bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

E652. The method or medicament as set forth in any one of E627-E651, wherein the cancer is one or more selected from the group consisting of renal cell carcinoma (RCC), bladder cancer, breast cancer, clear cell kidney cancer, head/neck squamous cell carcinoma (SCCHN), lung squamous cell carcinoma, malignant melanoma, non-small-cell lung cancer (NSCLC), ovarian cancer, pancreatic cancer, prostate cancer, small-cell lung cancer (SCLC) or triple negative breast cancer.

E653. The method or medicament as set forth in any one of E627-E652, wherein the cancer is one or more selected ted from the group consisting of a Heme malignancy and in some embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

E654. The antibody for use, method or medicament as set forth in any one of E1-E653, wherein at least one of the therapeutic agents is administered to a subject at intervals of once a day, once every two days, once every three days, once a week, once every two weeks, once every three weeks, once every four weeks, once every 30 days, once every five weeks, once every six weeks, once a month, once every two months, once every three months, or once every four months.

E656. The use of an antibody of any one of E1-E71, E77-E143, E144-193, E199-259, E265-E330, E336-E381, E387-E435, E445-E484, E500-E557, E596-E612, or E619-E625 for the manufacture of a medicament for us in the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, and non-alcoholic steatohepatitis (NASH), prurigo nodularis, keloids, bullous pemphigoid, chronic urticaria, IPF, scleroderma, systemic sclerosis, fungal keratitis, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL), Heme malignancy, acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), and small lymphocytic lymphoma (SLL).

E657. A pharmaceutical composition for the treatment of one or more selected from the group consisting of atopic dermatitis, asthma, cancer, COPD, food allergy, allergic rhinitis, eosinophilic esophagitis, chronic rhinosinusitis with nasal polyps, alopecia areata, and non-alcoholic steatohepatitis (NASH), prurigo nodularis, keloids, bullous pemphigoid, chronic urticaria, IPF, scleroderma, systemic sclerosis, fungal keratitis, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL), Heme malignancy, acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), and small lymphocytic lymphoma (SLL), comprising an antibody of any one of E1-E71, E77-E143, E144-193, E199-259, E265-E330, E336-E381, E387-E435, E445-E484, E500-E557, E596-E612, or E619-E625.

E658. An anti-disease agent comprising an antibody of any one of E1-E71, E77-E143, E144-193, E199-259, E265-E330, E336-E381, E387-E435, E445-E484, E500-E557, E596-E612, or E619-E625, wherein the disease is one or more selected from the group consisting of atopic dermatitis, asthma, cancer, COPD, food allergy, allergic rhinitis, eosinophilic esophagitis, chronic rhinosinusitis with nasal polyps, alopecia areata, and non-alcoholic steatohepatitis (NASH), prurigo nodularis, keloids, bullous pemphigoid, chronic urticaria, IPF, scleroderma, systemic sclerosis, fungal keratitis, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL), Heme malignancy, acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), and small lymphocytic lymphoma (SLL).

### BRIEF DESCRIPTION OF THE FIGURES

FIG. 1A depicts an exemplary sequence illustrating Pfabat numbering of light chain portions of the anti-IL-13 and anti-IL-4 binding arms. Position names appear vertically in single columns, and dashes (“-”) indicate gaps in the alignment. For example, residue L27D of the anti-IL-13 CDR-L1 is His, and the equivalent residue in the anti-IL-4 binding arm is Glu. Positions considered part of the CDRs are shown in bold text, and the corresponding amino acids are bold and underlined.

FIG. 1B depicts an exemplary sequence illustrating Pfabat numbering of heavy chain portions of the anti-IL-13, anti-IL-4, and anti-IL-33 binding arms. Position names appear vertically in single columns, and dashes (“-”) indicate gaps in the alignment. For example, residue H100C of the anti-IL-4 CDR-L1 is Phe, and the anti-IL-13 binding arm, with a shorter CDRH3, has no corresponding residue. Positions considered part of the CDRs are shown in bold text, and the corresponding amino acids are bold and underlined. The anti-IL-13 heavy chain fragment used in most multi-specific antibody sequences does not contain a full hinge or Fc. The Pfabat algorithm numbers the sequence as a free-standing Fab domain and does not number the small upper hinge fragment (“EPKSC” (SEQ ID NO: 102)), although it is included here manually (lower case, positions H226-H230) for reference.

FIG. 2 depicts a graph summarizing the results of LC/MS Analysis of IL4-1285 Antibody. Reveals that the Post-translational Modification is Located on the Light Chain. Mass spectrometry of peak 1 (P1) relative to peak 2 (P2) shows an 80 dalton (Da) species with a mass of 23755.0 Da present in P2. P1 refers to the “Main Peak” species isolated when analyzed by an ion exchange purification method and, also detected by using Light Chain LC/MS Analysis (after LysC+TCEP). P2 refers to “Peak 2” isolated by an ion exchange purification method and, also detected by using Light Chain LC/MS Analysis (after LysC+TCEP). Peak 2 has additional sulfation on Tyr(L27a) so it has an 80 Da shift in MS.

FIG. 3 depicts a graph comparing LC/MS analysis for IL4-1346 (hu3B9-VLv2.8) [lower two panels] and IL4-1285 (hu3B9-VLv2.0) antibody light chains [upper two panels]. The 0-linked sulfation at light chain CDR1 peptide (DRVTITCKASQSVDY) was identified by LC/MS analysis and the Y(L27d)F mutation removed the heterogeneity. The left panels on both top and bottom are peptide level mapping data (AspN enzyme). The right panels on top bottom are the subunit using a LysC method. The peptide mapping (AspN digestion) shows in the top left spectra with the DFD light chain does not contain the sulfation and the DYD light chain does have some of the sulfation (+79.9568 Da's). The subunit analysis shows the DFD light chain does not have the sulfated species (top right spectra) and the DYD light chain does have some of the sulfated Da species (bottom right spectra).

FIG. 4 depicts the changes incorporated into IL4-1359 (VH1 G07_VLv2.9) VH CDRH1 that contribute to the higher affinity for IL-4.

FIG. 5 depicts the change at Tyr(L27d)E that can be made to remove O-sulfation post-translational modification and further stabilize the IL-4/IL4-1285 (RA1-2 or hu3B9-VLv2.0) interface.

FIG. 6 depicts the alignment of IL-13 complexes revealing binding orientation of IL13-1283 (IMA-638 or hu13.2) versus IL13-1307 (hu13.4).

FIG. 7 depicts the CDRL1 differences in IL13-1307 (hu13.4) relative to IL13-1283 (IMA-638 or hu13.2) contribute to the higher affinity to human IL-13.

FIG. 8 depicts the CDRH3 amino acid differences between IL13-1307 (hu13.4) and IL13-1283 (IMA-638 or hu13.2) contribute to the higher affinity for IL-13.

FIG. 9 depicts how the presence of serine at residue 30 in IL13-1307 (hu13.4) CDRH1 contributes to higher affinity for IL-13 relative to IL13-1283 (IMA-638 or hu13.2).

FIG. 10 depicts how the aspartic acid at position 55 in IL13-1307 (hu13.4) CDRH2 instead of glycine at this position in IL13-1283 contributes to higher affinity for IL-13 relative to IL13-1283 (IMA-638 or hu13.2).

FIG. 11 depicts how the changes incorporated into IL13-0001 (1RVHC9-VLA4) VH CDRH3 contribute to the higher affinity for IL-13.

FIG. 12 depicts how the changes incorporated into IL13-0001 (1RVHC9-VLA4) VL CDRL1 contribute to the higher affinity for IL-13.

FIG. 13 depicts a graph showing how the increased potency of IL-33 neutralization correlates with increased polyreactivity.

FIG. 14A-B depicts high throughput off-rate screening of anti-TSLP variants. FIG. 14A: Off-rate screening sensorgram. R represents Response (nM). t0, t240 and t640 represent time points. Association index=Sample (Rt240-Rt0)/TSLP-0001 (Rt240-Rt0). Dissociation index=Sample (Rt640−Rt240)/TSLP-0001 (Rt640−Rt240). FIG. 14B: Spotfire plot of association index vs dissociation index. The variants inside the square box are the hits that met selection criteria.

FIG. 15 depicts an electrostatic surface plot of TSLP-0260 Fv region. The electrostatic surface plot was determined using the Poisson Boltzmann calculator Delphi. Here positive charge potential is shown in white and negative charge potential in black. The regions that were focused on to reduce viscosity were the negative charge patches circled in black dashed lines. These three patches contain parts of the CDRs L1-L2, L3-H2-H3 and H2 respectively.

FIG. 16 depicts a detail of the interface of antibody: TSLP. Mutation of VH-E99Y potentially introduces a new hydrogen bond network at the TSLP-Ab interface. Selected interface residues are labelled and shown as sticks. N71 and R149 are from TSLP. Dashed lines represent salt bridges and hydrogen bonds. Light grey with a-helix structure: TSLP. Dark grey: antibody. This also increases the overall packing.

FIG. 17 depicts a graph showing the viscosity analysis of anti-TSLP variants. The viscosity was measured by DLS bead-based method.

FIG. 18A, FIG. 18B, FIG. 18C, FIG. 18D, FIG. 18E, FIG. 18F, FIG. 18G, FIG. 18H, and FIG. 18I depict general schematics and nomenclature of trispecific Tri-Fab-Fc and Fab domains. FIG. 18A depicts a schematic drawing of Tri-Fab-Fc, indicating domains Fab1, Fab2, and Fab3, and the single Fab (SFab) and dual Fab (DFab) arms expressed in separate cells. FIG. 18: depicts a schematic drawing of conventional Fab. FIG. 18C depicts a schematic drawing of modified Fd. FIG. 18D depicts a schematic drawing of VH-VL swap. FIG. 18E depicts a schematic drawing of S1. FIG. 18F depicts a schematic drawing of S1Rev. FIG. 18G depicts a schematic drawing of CH-Ck swap. FIG. 18H depicts a schematic drawing of CH-Cl swap. FIG. 18. depicts an exploded view of Tri-Fab-Fc, indicating individual protein chains: the SFab HC(3) and SFab LC(3) chains, which together make up the SFab arm; and the DFab LC(1)-HC(2), modified Fd(1), and DFab LC(2) chains, which together make up the DFab arm. Note: VL denotes light chain variable domain; VH denotes heavy chain variable domain; Ck denotes kappa constant light chain domain, C denotes lambda constant light chain domain; CH1 denotes heavy chain constant domain 1. EPKSC (SEQ ID NO: 102): a stretch of amino acids from the IgG1 upper hinge region fused to C-terminus of CH1 to form disulfide bond to CL (Fab1).

FIG. 19A-B depicts the configuration and characterization of Tri-Fab-Fcs with native chain pairing in Fab1 Position. FIG. 19A and FIG. 19B depict the configuration of two native chain pairing Tri-Fab-Fc variants, in which all three Fabs have conventional chain pairing. S1: S1 mutations on CH1/CK. S1rev: S1rev mutations on CH1/CK. Positive charged (+) mutations as solid black circle. Negative charged (−) mutations as dotted black circle. FIG. 19C depicts graphs showing the analytical SECs of IL413TSLP-0003 and IL413TSLP-0004 after elution of Mab Select SuRe and after prepSEC superdex 200 purification. FIG. 19D and FIG. 19E depict graphs showing LCMS analysis of IL413TSLP-0003 and IL413TSLP-0004 respectively. The tables list each chain of a trispecific and corresponding theoretical chain mass. Numeric numbers 1-5 are placed in front of each chain to provide a shorthand description of chain composition for major peak in LCMS.

FIG. 20A, FIG. 20B, FIG. 20C, FIG. 20D, FIG. 20E, and FIG. 20F depict cartoons showing the configuration of CλS, CκS and VDS Tri-Fab-Fc variants, in which the Fab1 domain (anti-TSLP) contains the C domain fused to the C terminus of the VH1 domain, and the CH1 domain fused to the C terminus of the VL1 domain. The CλS configuration, illustrated by IL413TLSP-0001 (FIG. 20A) and IL413TSLP-0002 (FIG. 20B), has a Fab1 light chain with the structure VL-CH1 and the VH-CI at the N-terminus of the dual Fab arm. The VDS configuration, illustrated by IL413TLSP-0007 (FIG. 20C) and IL413TSLP-0008 (FIG. 20D), has Fab1 VI-CH1 at the N terminus of the dual Fab chain and a VH-CI Fab1 light chain. The CkS configuration, illustrated by IL13433-0021 (FIG. 20E) has Fab1 and Fab3 harboring the S1 and S1 rev mutations, respectively, Fab2 contains a VL-CH1 structure and the VH-CL is fused to the N-terminus of the Fc. The CkS configuration, illustrated by IL13433-0022 (FIG. 20F) has Fab1 and Fab3 harboring the S1 and S1 rev mutations, respectively, Fab2 contains a VL-CH1 structure that includes a SS elbow between the VL and CH1 domains, and the VH-CL is fused to the N-terminus of the Fc.

FIG. 21 depicts a graph showing the evaluation of the effect of domain geometry on Tri-Fab-Fc binding activity by bridging ELISA. TPP-9662 is an IL-4/IL-13 bispecific antibody with S1/S1rev and KiH. mab8.8 is a negative control antibody.

FIG. 22A-B depicts the improvement of Tri-Fab-Fc chain pairing by adjusting DNA ratio in transient transfection. FIG. 22A depicts a NuPAGE Bis-tris gel analysis. R: reducing. NR: non-reducing. FIG. 22B depicts a graph showing aSEC analysis.

FIG. 23-23D depicts a cartoon of Tri-Fab-Fc variants engineered with modified Fd format and S1/S1rev complementary mutations.

FIG. 24 depicts non-reduced and reduced stain free SDS-PAGE analysis of stable CHO produced Tri-Fab-Fc.

FIG. 25A-25B depicts a cartoon of the configuration of representative Tri-Fab-Fc trispecifics with CκS or mFd Pairing of Fab1 using charge-based heterodimerization. RRR or EEE charge mutations: two RR/EE are at hinge region and one R/E is at CH3. Anti-IL-13 domain is designed to be either CkS (exemplified by IL413TSLP-0251) or mFd (exemplified by IL413TSLP-0252).

FIG. 26 depicts a carton of anti-IL-13/IL-4 dual Fab EEE arm designs with Anti-IL-13 domain as mFd at Fab1 position and anti-IL-4 domain at Fab2 position.

FIG. 27 depicts a cartoon of anti-IL-13/IL-4 dual Fab EEE CB arm designs with anti-IL-4 binding domain as mFd at Fab1 position and anti-IL-13 domain at Fab2 position.

FIG. 28A-28B depicts a cartoon of anti-IL-13/IL-4 dual Fab EEE arm designs with CκS at Fab 1 position.

FIG. 29A-29C depicts a graph showing that Tri-Fab-Fc IL13433-0006 can simultaneously bind human IL-4, IL-13, and IL-33.

FIG. 30 depicts an SPR sensorgram of IL413p40-0705 with human IL-4, IL-13 and IL-23 in various orders of injection.

FIG. 31A-31D depicts the dual cell design Tri-Fab-Fc variants with mFd format and S1-S1rev complementary mutations.

FIG. 32 depicts a Tri-Fab-Fc engineered with modified Fd format and produced using the single cell process.

FIG. 33 depicts a graph showing the viscosity of IL413p40-0698 and IL413p40-0700 measured by differential light scattering (DLS).

FIG. 34 depicts anti-IL-4/13/33 Tri-Fab-Fc IV pharmacokinetics in the Tg32 mouse. Antibody designations are abbreviated to their four-digit references as follows: 1042 refers to IL13433-1042, 1258 refers to IL13433-1258, 1261 refers to IL13433-1261, 1270 refers to IL13433-1270, 1275 refers to IL13433-1275, and Anti-IL-33 Ab LS refers to IL33-0232 VH and VL with Fc domains further comprising the LS mutation as herein described.

FIG. 35A-35C depicts a graph showing the binding of Trispecifics to human, cynomolgus monkey, mouse, and rat IL-4 by surface plasmon resonance. Trispecifics: (a) IL13433-1258, (b) IL413TSLP-1024, (c) IL413P40-0705 were immobilized onto a CM5 sensor chip and binding of 200 nM human (hu), cynomolgus monkey (cy), mouse (mu), rabbit (rb) or rat (rt) IL-4 was determined by surface plasmon resonance.

FIG. 36A-36C depicts a graph showing the binding of Trispecifics to human, cynomolgus monkey, mouse, and rat IL-13 by surface plasmon resonance. Trispecifics: (a) IL13433-1258, (b) IL413TSLP-1024, (c) IL413P40-0705 were immobilized onto a CM5 sensor chip and binding of 200 nM human (hu), cynomolgus monkey (cy), mouse (mu), rabbit (rb) or rat (rt) IL-13 was determined by surface plasmon resonance.

FIG. 37 is a cartoon depicting different Fab positions within Tri-Fab-Fc construct with modified Fd (mFd) design.

FIG. 38 depicts an SPR sensorgram of Tri-Fab-Fc IL413p40-0705 with human (hu), cynomolgus monkey (cy), mouse (mu), rat (rt) and rabbit (rb) IL-12, IL-23, IL-4, and IL-13.

FIG. 39 depicts a graph showing the species specificity for IL-33 binding to trispecific IL13433-1258 by surface plasmon resonance.

FIG. 40 depicts a graph showing the binding of short form and long form TSLP to TSLP-0001, mAb TSLP-0875, and trispecific IL413TSLP-1024 by Surface Plasmon Resonance.

FIG. 41 depicts a graph showing the bioactivity of short form and long form TSLP in inducing monocyte TARC Production. Mononuclear cells isolated from human peripheral blood were incubated overnight at 37° C. with 0.31 pg/mL-20 ng/ml recombinant human TSLP (Pfizer), short form TSLP (TSLPIf Avi V5 His 10 Biotin), or long form TSLP (TSLPIf Avi V5 His 10 Biotin). TARC production in cell supernatants was quantitated by MSD. Data presented as mean+/−S.D. EC50 values for hTSLP, sfTSLP, and IfTSLP are 0.3680, N/A, and 7.472 ng/mL respectively.

FIG. 42 depicts a graph showing the binding of human, cynomolgus monkey, mouse, and rat TSLP to trispecific IL413TSLP-1024 by Surface Plasmon Resonance. Biotinylated human (hu), cynomolgus monkey (cy), mouse (mu), or rat (rt) TSLP was captured onto a Biotin CAP sensor chip, and binding of IL413TSLP-1024 trispecific was determined by surface plasmon resonance.

FIG. 43 depicts graphs showing the simultaneous binding of IL-4, IL-13, and TSLP to IL413TSLP-1024, by Surface Plasmon Resonance.

FIG. 44A-44B depicts graphs showing the average CT26 tumor volumes over time and at the end of the study. The results of a study wherein CT26 tumor-bearing Balb/c mice were dosed with the compounds listed in Table 88. (A) Average volume (mm3) and standard error of the mean of implanted tumors over time starting at day 9 (first day of drug injection) for each treatment group. (B) Volumes (mm3) of implanted tumors on day 22 post-implant (the final day of the study). The bars and whiskers represent the average and standard deviation for each treatment group. Each dot represents the final tumor volume for each mouse in that treatment group. Treatment with anti-PD-1 antibody, anti-IL-4 antibody plus mIL13Rα2-mFc or anti-IL-4 antibody plus mIL13Rα2-mFc plus anti-PD-1 antibody did not significantly alter the tumor growth characteristics compared to isotype treated animals. In contrast, mice treated with anti-IL-4 plus anti-TSLP antibodies plus mIL13Rα2-mFc (40% tumor growth inhibition, p=0.043) or with anti-IL-4 plus anti-TSLP plus anti-PD-1 antibodies plus mIL13Rα2-mFc (54% tumor growth inhibition, p=0.002) exhibited significant tumor growth inhibition versus isotype.

FIG. 45 depicts graphs showing the CT26 tumor volumes per mouse in each treatment group over time. The volumes (in mm3) of subcutaneously implanted CT26 tumors over time starting at day 9 (first day of drug injection) for individual mice in each treatment group.

FIG. 46A-46B depicts graphs showing that neutralization of IL-4 with mAb IL4-1040 or TSLP with mAb TSLP-0875 prevents suppression of interferon gamma secretion by primary human tumor-reactive T cells. Interferon gamma secretion from primary, A375-reactive human T cells polarized and restimulated as described in Example 87 with the treatments listed in Table 2. (A) Shows Interferon gamma secretion from six distinct donors tested across multiple days. T cells were polarized and restimulated with either an isotype antibody alone, an isotype antibody and 0.8 ng/mL recombinant human IL-4 or mAb IL4-1040 and 0.8 ng/mL recombinant human IL-4. (B) Shows Interferon gamma secretion from the same six donors as in (A) polarized and restimulated with either an isotype antibody, an isotype antibody and 0.8 ng/mL recombinant human TSLP or mAb TSLP-0875 and 0.8 ng/mL recombinant human TSLP. Data in (A & B) are presented as the mean and standard deviation. Statistical tests in (A & B) are ANOVA with p-values from post-hoc Šidák's multiple comparison testing shown above the bars.

FIG. 47A-47D depicts graphs showing that neutralization of IL-4 with mAb IL4-1040 and/or TSLP with mAb TSLP-0875 improved T cell-mediated control of human A375 cancer cell growth in vitro, which correlated with Interferon gamma secretion. Primary human T cells were polarized and restimulated as described in Example 87. Growth curves of A375 tumor cells during T cell restimulation were generated as described in Example 88. (A) Primary human T cells were polarized and restimulated with: (A) an isotype antibody alone, isotype antibody and 0.8 ng/mL recombinant human IL-4 or mAb IL4-1040 and 0.8 ng/mL recombinant human IL-4; (B) an isotype antibody alone, isotype antibody and 0.8 ng/mL recombinant human TSLP or mAb TSLP-0875 and 0.8 ng/mL recombinant human TSLP; (C) an isotype antibody alone, isotype antibody and 0.8 ng/mL each recombinant human IL-4 and TSLP or a combination of mAb1040, mAb and 0.8 ng/mL each recombinant human IL-4 and TSLP. (D) Interferon gamma in conditioned media collected on day 5 of restimulation was quantified as described in Example 87. Levels of secreted Interferon gamma were correlated with normalized A375 AUC from the same wells. (A-C) are data from donor 1923 and (D) is from donor 8385. Data in (A-C) are presented as the mean and standard deviation of six technical replicates and exemplify data from three different donors. Data in (D) is presented as the mean of six technical replicates and exemplify data from four donors.

FIG. 48 depicts graphs showing that recombinant human IL-4 or the combination of IL-4 and TSLP reduced the frequency of A375 polarized primary human CD8 T cells expressing both Perforin and Granzyme B. The proportion of A375 polarized CD8 T cells expressing both Perforin and Granzyme B determined by flow cytometry as described in Example 89. Data are presented as the mean and standard deviation of three technical replicates and are representative of data from two distinct donors.

FIG. 49A-49B depicts graphs showing that Neutralization of IL-4- and IL-13-induced CCL17 secretion from the human clear cell renal cell carcinoma line 769-P by trispecifics IL413TSLP-1028 and IL413TSLP-1037. 769-P human clear cell renal cell carcinoma cells were incubated for approximately 20 hours at 37° C. with recombinant human IL-4 (A) or IL-13 (B), along with dilutions of trispecific IL413TSLP-1028 or IL413TSLP-1037. CCL17 secretion was quantified by Legendplex and concentrations extrapolated from standard curves.

### DETAILED DESCRIPTION

Provided herein are antibodies that specifically bind to IL-4, antibodies that specifically bind to IL-13, antibodies that specifically bind to IL-33, antibodies that specifically bind to TSLP, and multispecific antibodies that specifically bind to IL-4 and IL-13 together with one of IL-33, TSLP, and p40. Also provided herein are related nucleic acids, compositions, and methods of making and using the antibodies.

### General Techniques

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined, all terms of art, notations and other scientific terms or terminology used herein are intended to have the meanings commonly understood by those of skill in the art to which this invention pertains. For example, the term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone). In some cases, terms with commonly understood meanings are defined herein for clarity and/or for ready reference, and the inclusion of such definitions herein should not necessarily be construed to represent a substantial difference over what is generally understood in the art.

As used herein, the singular forms “a”, “an” and “the” include their corresponding plural references unless the context clearly dictates otherwise.

As used herein, the numeric ranges are inclusive of the numbers defining the range.

Reference to “about” a value or parameter herein includes (and describes) embodiments that are directed to that value or parameter per se, as well as to values or parameters that may be as much as 10% below or above the stated numerical value for that parameter. For example, as dose of “about 5 mg/kg” includes 5 mg/kg and also any value between 4.5 mg/kg and 5.5 mg/kg. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g. monospecific, bispecific, trispecific, multispecific), and includes portions of intact antibodies that retain the ability to bind to a given antigen (e.g. an “antigen-binding fragment”), and any other modified configuration of an immunoglobulin molecule that comprises an antigen binding site.

An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody or the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the conformational definition, and the Pfabat numbering method set forth in Example 1.

The Kabat definition is a standard for numbering the residues in an antibody and is often used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches but will nonetheless overlap with at least a portion of the Kabat CDRs, although they may be shortened or lengthened in light of prediction or experimental findings that particular residues or groups of residues do not significantly impact antigen binding. The numbering system used in this application is the Pfabat Numbering method set forth in Example 1.

As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, conformational definitions, or Pfabat Method. The antibodies of this application are numbered according to a modified version of the Kabat numbering system and is set forth in more detail in Example 1.

The term “hinge region” as used herein includes the meaning known in the art, which is illustrated in, for example, Janeway et al., ImmunoBiology: the immune system in health and disease, Elsevier Science Ltd., NY (4th ed., 1999); Bloom et al., Protein Science, 6:407-415, 1997; and Humphreys et al., J. Immunol. Methods, 209:193-202, 1997.

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL).

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain or “wild-type Fc chain” refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification yet retains at least one effector function of the wild-type Fc chain. In some embodiments, the variant Fc chain has at least one amino acid substitution compared to a wild-type Fc chain e.g., from about one to about ten amino acid substitutions, and preferably, from about one to about five amino acid substitutions in a wild-type Fc chain. The variant Fc chain herein will preferably possess at least about 80% sequence identity with a wild-type Fc chain, and most preferably, at least about 90% sequence identity therewith, more preferably, at least about 95%, at least about 96%, at least about 97%, at least about 98%, at least about 99% sequence identity therewith. In some embodiments, an Fc chain comprises part or all of a wild-type hinge region (generally at its N-terminal). In some embodiments, an Fc polypeptide does not comprise a functional or wild-type hinge region.

### Nomenclature

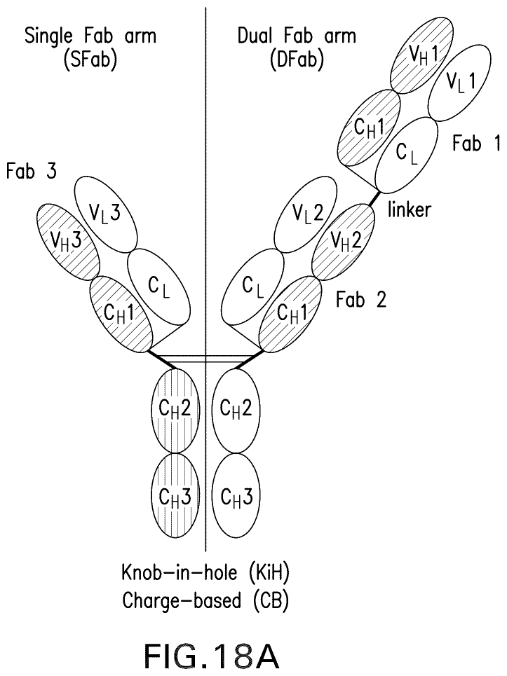

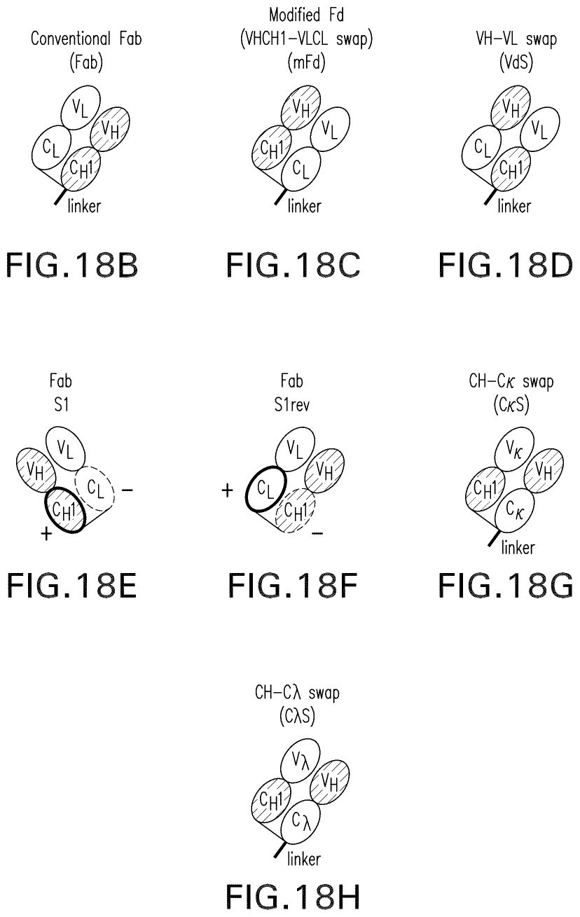

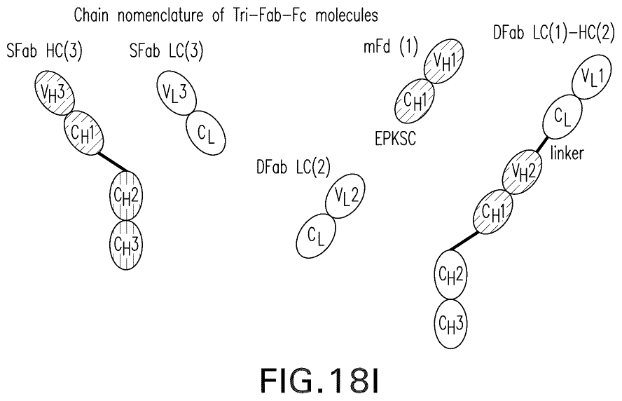

The antibody domains disclosed herein may be prefixed with an indicator of the respective target antigen that that domain is most closely associated with. The prefixes are purely added to provide a shorthand clarity in distinguishing between the different domains recited throughout the disclosure, and do not replace or conflict with the actual function or structure of the various domains. For example, IL4-VH refers to the variable heavy domain of antibody that comprises CDRs that can specifically bind to IL-4. An IL4-CH1 refers to the CH1 domain located C-terminal to an IL-4 variable domain, whether that variable domain is a variable light domain (IL4-VL) or a variable heavy domain (IL4-VH). As described throughout the disclosure, the domains of the antibodies of the disclosure may be combined or fused with domains from other antibodies. Thus, for example, an IL4-VH may be present on a polypeptide chain with an IL13-VL, and each may independently, or together, be present in the same antibody, and that polypeptide may be referred to as both an IL4-VH and an IL13-VL (e.g. see FIG. 18). Again, by way of example, an IL4-VH bearing polypeptide is any polypeptide that comprises an IL4-VH domain—i.e. a VH domain comprising CDRs that specifically bind to IL-4. An IL4-VH bearing polypeptide may comprise a hinge region, CH1 domain, CH2 domain, and CH3 domain, as in a standard IgG format, or may comprise various domains as described herein, such as an IL13-VL, together with a CH1 and CL domain fused either to the IL4-VH or IL13-VL (see FIG. 18 for illustrations of different combinations of antibody domains). For example, the DFab LC(1)-HC(2) arm (see FIG. 18I) of IL41333-1258, IL413TSLP-1024, and IL413P40-0705 each comprises, from N-terminal to C-terminal, IL13-VL, CL, linker, IL4-VH, CH1, CH2, and CH3, and may be referred to as both an IL4-VH bearing polypeptide and an IL13-VL bearing polypeptide. Further, framework regions may be referred to by the targets to which their CDRs are specific for. For example, the framework region of IL-4 antibodies of the disclosure may be referred to as an IL4-VL framework or IL4-VH framework. The skilled person will understand that these designations neither confer nor imply any relationship between that framework region and the target that the CDRs specifically bind to. Thus, the IL4-VL framework region may be derived from a human germline DPK9 sequence that is identical to a known human DPK9 germline sequence. This nomenclature is not limited to the specific antibodies exemplified in this paragraph, and applies across all antibodies and antibody combinations possible within the disclosure.

The antibodies IL13433-1258, IL134TSLP-1024, and IL134p40-0705 may each be described as comprising a first, second, third, fourth, and fifth polypeptide chain, where the second polypeptide comprises from N-terminus to C-terminus, (VL-1)-(CL-1)-(linker)-(VH-2)-(CH1-2)-(second hinge)-(second CH2)-(second CH3) and the fifth polypeptide comprises from N-terminus to C-terminus, (VH1)-(CL-1); and the fourth polypeptide comprises (VL-2)-(CL-2); the first polypeptide comprises, from N-terminus to C-terminus, (VH-3)-(CH1-3)-(first hinge)-(first CH2)-(first CH3); and the third polypeptide comprises (VL-3)-(CL-3). In each case, the VL-1 and VH-1 is IL13-VL and IL13-VH; the VL-2 and VH-2 is IL4-VL and IL4-VH, and the VH-3 and VL-3 is IL33-VH and IL33-VL; or TSLP-VH and TSLP-VL; or p40-VH and p40-VL.

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

An “antigen” refers to the molecular entity used for immunization of an immunocompetent vertebrate to produce the antibody that recognizes the antigen or to screen an expression library (e.g., phage, yeast or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition or alternatively, during the discovery process, the generation and characterization of antibodies may elucidate information about desirable epitopes. From this information, it is then possible to competitively screen antibodies for binding to the same epitope.

As used herein, the term “binding affinity,” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. In particular, the term “binding affinity” is intended to refer to the dissociation rate of a particular antigen-antibody interaction. The K_{D} is the ratio of the rate of dissociation, also called the “off-rate (k_{off}),” to the association rate, or “on-rate (kₒₙ)”. Thus, K_{D} equals k_{off}/kₒₙ and is expressed as a molar concentration (M). It follows that the smaller the K_{D}, the stronger the affinity of binding. Therefore, a K_{D} of 1 pM indicates weak binding affinity compared to a K_{D} of 1 nM. K_{D} values for antibodies can be determined using methods well established in the art. One method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Determinations of the association and dissociation rate constants, kₒₙ and k_{off} respectively, to determine K_{D} and other ratios, may be made, for example, using a surface plasmon resonance-based biosensor to characterize an analyte/ligand interaction under conditions where the analyte is monovalent with respect to binding a ligand that is immobilized at low capacity onto a sensor surface via a capture reagent. The analysis may be performed, for example, using a kinetic titration methodology as described in Karlsson et al., Anal. Biochem 349, 136-147, 2006, or using a multi-cycle kinetics analysis. The sensor chip, capturing reagent, and assay buffer employed for a given assay are chosen to give stable capture of ligand onto the sensor surface, minimize non-specific binding of the analyte to the surfaces, and yield analyte-binding responses that are appropriate for kinetic analysis, per the recommendations in Myszka, J. Mol. Recognit 12, 279-284, 1999. The analyte-binding responses per analyte/ligand interaction are double referenced and fit to a 1:1 Langmuir “mass transport limited model” with kₐ, k_{d} and Rₘₐₓ as global parameters as described in Myszka & Morton et al., Biophys. Chem 64, 127-137 (1997). The equilibrium dissociation constant, K_{D}, is deduced from the ratio of the kinetic rate constants, K_{D}=k_{off}/kₒₙ. Such determinations preferably take place at 25° C. or 37° C. Typically, the rate constants (kₒₙ or kₐ and k_{off} or k_{d}) and equilibrium dissociation constants are measured using whole antibody and monomeric. Another method for determining the K_{D} of an antibody is by using Bio-Layer Interferometry, typically using OCTET® technology (Octet QK^{e} system, ForteBio). Alternatively, or in addition, a KinExA (Kinetic Exclusion Assay) assay, available from Sapidyne Instruments (Boise, ID) can also be used.

As used herein, the terms “immunospecifically binds,” “immunospecifically recognizes,” “specifically binds,” “specifically recognizes” and analogous terms refer to molecules e.g., binding domains that specifically bind to an antigen (e.g., epitope or immune complex) and do not specifically bind to another molecule. A molecule that specifically binds to an antigen may bind to other peptides or polypeptides with lower affinity as determined by assays known in the art e.g., immunoassays, BIACORE™ or other assays. Preferably, molecules that specifically bind an antigen do not cross-react with other proteins.

The terms “specific binding” or “specifically binding” when used in reference to the interaction of an antibody and a protein or peptide refers to an interaction that is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

In certain embodiments “specifically binds” means, for instance, that an antibody binds a protein with a K_{D} of about 0.1 nM or less, but more usually less than about 1 μM. In certain embodiments, “specifically binds” means that an antibody binds a target at times with a K_{D} of at least about 0.1 μM or less, at other times at least about 0.01 μM or less, and at other times at least about 1 nM or less. Because of the sequence identity between homologous proteins in different species, specific binding can include an antibody that recognizes a protein in more than one species. Likewise, because of homology within certain regions of polypeptide sequences of different proteins, specific binding can include an antibody that recognizes more than one protein. It is understood that, in certain embodiments, an antibody or binding moiety that specifically binds a first target may or may not specifically bind a second target. As such, “specific binding” does not necessarily require (although it can include) exclusive binding, i.e. binding to a single target. Thus, an antibody may, in some embodiments, specifically bind more than one target. In certain embodiments, multiple targets may be bound by the same antigen-binding site on the antibody. For example, an antibody may, in certain instances, comprise two identical antigen-binding sites, each of which specifically binds the same epitope on two or more proteins. In certain alternative embodiments, an antibody may be multispecific and comprise at least two antigen-binding sites with differing specificities. By way of non-limiting example, a bispecific antibody may comprise one antigen-binding site that recognizes an epitope on one protein and further comprise a second, different antigen-binding site that recognizes a different epitope on a second protein. Generally, but not necessarily, reference to binding means specific binding.

An antibody that specifically binds to an antigen may bind to other peptides or polypeptides with lower affinity as determined by assays known in the art e.g. immunoassays, BIACORE™, or other assays. Preferably, the antibody that specifically binds an antigen does not cross-react with other proteins.

The terms “non-specific binding” or “background binding” when used in reference to the interaction of an antibody and a protein or peptide refers to an interaction that is not dependent on the presence of a particular structure (i.e., the antibody is binding to proteins in general rather that a particular structure such as an epitope).

A neutralizing or “blocking” antibody refers to an antibody whose binding to one or more selected from the group consisting of IL-4, IL-13, IL-33, TSLP, and p40 one or both of (i) interferes with, limits, or inhibits the interaction between IL-4, IL-13, IL-33, TSLP, and p40 respectively and a suitable ligand; or (ii) results in inhibition of at least one biological function of IL-4, IL-13, IL-33, TSLP, or p40 binding as appropriate. Assays to determine neutralization by an antibody of the disclosure are well-known in the art.

As used herein, an “antibody that binds to” a target; an “antibody that recognizes” a target; an “antibody that specifically binds” a target; an “anti-target antibody;” an “anti-target antibody molecule;” a “target antibody”; or the like, comprises a molecule that contains at least one binding domain that specifically binds to the target.

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

A “bispecific antibody” refers to a molecule that has binding specificity for at least two different epitopes. In some embodiments, bispecific antibodies can bind simultaneously two different antigens. In other embodiments, the two different epitopes may reside on the same antigen.

As used herein, a “trispecific antibody” is an antibody that has binding specificity for three different epitopes. In some embodiments, trispecific antibodies can bind simultaneously three different antigens. In other embodiments, the three different epitopes may reside on the same antigen.

As used herein, a “multispecific antibody” is an antibody that has binding specificity for at least two different epitopes. In some embodiments, multispecific antibodies can bind simultaneously at least two different antigens. In other embodiments, the at least two different epitopes may reside on the same antigen.

As used herein, the term “IL-4/IL-13/IL-33 trispecific antibody” or “IL-4/IL-13/IL-33 multispecific antibody” refers to a molecule designed to specifically bind to IL-4, IL-13, and IL-33. As used herein, the term “IL-4/IL-13/TSLP trispecific antibody” or “IL-4/IL-13/TSLP multispecific antibody” refers to a molecule designed to specifically bind to IL-4, IL-13, and TSLP. As used herein, the term “IL-4/IL-13/p40 trispecific antibody” or “IL-4/IL-13/p40 multispecific antibody” refers to a molecule designed to specifically bind to IL-4, IL-13, and p40.

The term “half maximal effective concentration (EC₅₀)” refers to the concentration of a therapeutic agent which causes a response halfway between the baseline and maximum after a specified exposure time. The therapeutic agent may cause inhibition or stimulation. The EC₅₀ value is commonly used, and is used herein, as a measure of potency.

The term “inhibitory concentration” (IC₅₀)” refers to the concentration of an inhibitor at which 50% of inhibition in its activity is achieved. The therapeutic agent may cause inhibition or stimulation. The IC₅₀value is commonly used, and is used herein, as a measure of potency.

An “agonist” refers to a substance which promotes (i.e., induces, causes, enhances, or increases) the biological activity or effect of another molecule. The term agonist encompasses substances (such as an antibody) which bind to a molecule to promote the activity of that molecule.

An “antagonist” refers to a substance that prevents, blocks, inhibits, neutralizes, or reduces a biological activity or effect of another molecule, such as a receptor. The term antagonist encompasses substances (such as an antibody) which bind to a molecule to prevent or reduce the activity of that molecule.

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

A “vector” refers to a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc). Vectors may include DNA or RNA. An “expression vector” as used herein refers to a vector that includes at least one polypeptide-encoding gene, at least one regulatory element (e.g. promoter sequence, poly(A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers and terminator). For translation, one or more translational controlling elements may also be included such as ribosome binding sites, translation initiation sites, and stop codons.

An “isolated” molecule (e.g. an isolated antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art.

As used herein, the term “linker” refers to an amino acid sequence of two or more amino acids in length. The linker can consist of neutral polar or nonpolar amino acids. A linker can be, for example, 2 to 100 amino acids in length, such as between 2 and 50 amino acids in length, for example, 3, 4, 5, 6, 8, 10, 12, 15, 20, 25, 30, 35, 40, 45, or 50 amino acids in length. A linker can be “cleavable,” for example, by auto-cleavage, or enzymatic or chemical cleavage. Cleavage sites in amino acid sequences and enzymes and chemicals that cleave at such sites are well known in the art and are also described herein. In some aspects, the linker comprises one or more repeating units of a sequence comprising glycine and serine residues. In some aspects, the linker comprises one or more repeating units of G₄S. In some aspects, the linker comprises (G₄S)₁₋₃. In some aspects, the linker is GGGGS (SEQ ID NO: 10).

As used herein, the term “disulfide bond” or “cysteine-cysteine disulfide bond” refers to a covalent interaction between two cysteines in which the sulfur atoms of the cysteines are oxidized to form a disulfide bond. The average bond energy of a disulfide bond is about 60 kcal/mol compared to 1-2 kcal/mol for a hydrogen bond. In the context of this invention, the cysteines which form the disulfide bond are within the framework regions of the single chain antibody and serve to stabilize the conformation of the antibody. Cysteine residues can be introduced, e.g., by site directed mutagenesis, so that stabilizing disulfide bonds can be made within the molecule.

As used herein, the terms “linked,” “fused” and “fusion” are used interchangeably to refer to the joining together of two more elements or components, by whatever means including chemical conjugation or recombinant means.

As used herein, the term “covalently linked” means that the specified moieties are either directly covalently bonded to one another, or else are indirectly covalently joined to one another through an intervening moiety or moieties, such as a linking peptide or moiety.

As used herein, the term “connected to” refers to the co-linear, covalent linkage or attachment of two or more proteins, polypeptides, or fragments thereof via their individual peptide backbones. For example, one polypeptide may be connected to another polypeptide through genetic expression of a single polynucleotide molecule encoding those two polypeptides in-frame. Such genetic fusion results in the expression of a single contiguous protein comprising both polypeptides.

As used herein, the term “modification” refers to an amino acid substitution, insertion, or deletion in a polypeptide sequence, an alteration to a moiety chemically linked to a protein, or a modification of a function of a protein, e.g., an antibody. For example, a modification may be an altered function of an antibody, or an altered carbohydrate structure attached to a protein. As used herein, an “amino acid modification” refers to a mutation (substitution), insertion (addition), or deletion of one or more amino acid residue in an antibody. The term “amino acid mutation” denotes the substitution of at least one existing amino acid residue with another different amino acid residue (e.g. the replacing amino acid residue). The term “amino acid deletion” denotes the removal of at least one amino acid residue at a predetermined position in an amino acid sequence. For example, the mutation L234A denotes that the amino acid residue lysine at position 234 in an antibody Fc-region is substituted by the amino acid residue alanine (substitution of lysine with alanine), (numbering according to the EU index numbering system).

The term “agent” is used herein to denote a biological macromolecule, an extract made from biological materials, a mixture of biological macromolecules, a chemical compound, a mixture of chemical compounds, or a mixture of chemical compounds and biological macromolecules. The term “therapeutic agent” refers to an agent that has biological activity.

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As used herein, the “first polypeptide” is any polypeptide which is to be associated with a second polypeptide. The first polypeptide and second polypeptide meet at an interface. In addition to the interface, the first polypeptide may comprise one or more additional domains, such as “binding domains” (e.g., an antibody variable domain, receptor binding domain, ligand binding domain or enzymatic domain) or antibody constant domains (or parts thereof) including CH2, CH1 and CL domains. Normally, the first polypeptide will comprise at least one domain which is derived from an antibody. This domain conveniently is a constant domain, such as the CH3 domain of an antibody and can form the interface of the first polypeptide. Exemplary first polypeptides include antibody heavy chain polypeptides, chimeras combining an antibody constant domain with a binding domain of a heterologous polypeptide, receptor polypeptides, ligand polypeptides, and antibody variable domain polypeptides (e.g., bispecific antibodies).

In addition to the interface, the second polypeptide may comprise additional domains such as a “binding domain” (e.g., an antibody variable domain, receptor binding domain, ligand binding domain or enzymatic domain), or antibody constant domains (or parts thereof) including CH2, CH1 and CL domains. Normally, the second polypeptide will comprise at least one domain which is derived from an antibody. This domain conveniently is a constant region, such as the CH3 domain of an antibody and can form the interface of the second polypeptide. Exemplary second polypeptides include antibody heavy chain polypeptides, chimeras combining an antibody constant domain with a binding domain of a heterologous polypeptide, and antibody variable domain polypeptides (e.g., bispecific antibodies).

A “polynucleotide” or “nucleic acid,” (used interchangeably herein) refers to a chain of nucleotides of any length, and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside.

As used herein, nucleic acids are written left to right in 5′ to 3′ direction; amino acid sequences are written left to right in amino to carboxy orientation, respectively. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include mature and immature mRNAs, such as precursor mRNAs (pre-mRNA) or heterogeneous nuclear mRNAs (hnRNA) and mature mRNAs. Additional coding or non-coding sequences may, but need not, be present within a polynucleotide of the present invention, and a polynucleotide may, but need not, be linked to other molecules or support materials.

It will be appreciated by those of ordinary skill in the art that, as a result of the degeneracy of the genetic code, there are many nucleotide sequences that encode amino acid sequences provided herein. Polynucleotides that vary due to differences in codon usage are specifically contemplated by the present invention.

A “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, and leucine; aspartic acid and glutamic acid; asparagine and glutamine; serine and threonine; lysine and arginine; and phenylalanine and tyrosine.

The invention also encompasses modifications to the antibodies provided herein, including functionally equivalent antibodies which do not significantly affect their properties and variants which have enhanced or decreased activity or affinity. For example, the amino acid sequence may be mutated to obtain an antibody with the desired binding affinity to IL-4, IL-13, IL-33, TSLP, or p40. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown below as residues within the same numbered groups (1-6). If such substitutions result in a change in biological activity, then more substantial changes, denominated “exemplary substitutions” as further described below in reference to amino acid classes, may be introduced and the products screened.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring amino acid residues are divided into groups based on common side-chain properties:
(1) Non-polar: Norleucine, Met, Ala, Val, Leu, lie;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. In still other embodiments, the CDR domain is either or both of the VH CDR3 or VL CDR3.

Modifications also include glycosylated and non-glycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, Chem. Immunol. 65:111-128, 1997; Wright and Morrison, TibTECH 15:26-32, 1997). The oligosaccharide side chains of the immunoglobulins affect the protein's function (Boyd et al., Mol. Immunol. 32:1311-1318, 1996; Wittwe and Howard, Biochem. 29:4175-4180, 1990) and the intramolecular interaction between portions of the glycoprotein, which can affect the conformation and presented three-dimensional surface of the glycoprotein (Jefferis and Lund, supra; Wyss and Wagner, Current Opin. Biotech. 7:409-416, 1996). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., Mature Biotech. 17:176-180, 1999).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g., antibodies, as potential therapeutics is rarely the native cell, variations in the glycosylation pattern of the antibodies can be expected (see, e.g., Hse et al., J. Biol. Chem. 272:9062-9070, 1997).

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g. algorithms), which are well known in the art.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

The term ‘excipient’ refers to any material which, which combined with an active ingredient of interest (e.g. antibody), allow the active ingredient to retain biological activity. The choice of excipient will to a large extent depend on factors such as the mode of administration, the effect of the excipient on solubility and stability, and the nature of the dosage form. As used herein, “excipient” ” includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Another particular example of cancer includes renal cell carcinoma.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents. Chemotherapeutic agents are further described elsewhere herein.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” a cancer as used herein means to administer a combination therapy of at least a first therapeutic agent and second therapeutic agent to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to National Cancer Institute (NCI) standards, a T/C less than or equal to 42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination of the invention is any of partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow, and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naive or untreated subjects or patients. In some embodiments, response to a combination of the invention is any of PR, CR, PFS, DFS, OR, or OS that is assessed using Response Evaluation Criteria in Solid Tumors (RECIST) 1.1 response criteria. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

The terms “treatment regimen”, “dosing protocol” and dosing regimen are used interchangeably to refer to the dose and timing of administration of each therapeutic agent in a combination of the invention.

As used herein, “treatment” is an approach for obtaining beneficial or desired clinical results. Beneficial or desired clinical results include, but are not limited to, one or more of the following: reducing the proliferation of (or destroying) neoplastic or cancerous cells, inhibiting metastasis of neoplastic cells, shrinking or decreasing the size of tumor.

As used herein, the term “treat,” “treating” or “treatment” is an approach for obtaining beneficial or desired clinical results. For the purpose of the present invention, treatment is defined as the administration of an anti-IL-4, anti-IL-13, anti-IL-33, anti-TSLP, anti-IL-4/anti-IL-13 bispecific, anti-IL-4/anti-IL-13 multispecific, anti-IL-4/anti-IL13/anti-IL-33 trispecific; anti-IL-4/anti-IL13/anti-TSLP trispecific; anti-IL-4/anti-IL13/anti-p40 trispecific to a subject, e.g., a patient. Such administration can be e.g., by direct administration to the subject or by application to an isolated tissue or cell from a subject which is returned to the subject. The antibody molecule can be administered alone or in combination with one or more agents. The treatment can be to cure, heal, alleviate, relieve, alter, remedy, ameliorate, palliate, improve or affect the disorder, the symptoms of the disorder or the predisposition toward the disorder, e.g., one or more diseases or conditions selected from the group consisting of atopic dermatitis, atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, Crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa

The terms “prevent” or “prevention” refer to one or more of delay of onset, reduction in frequency, or reduction in severity of at least one sign or symptom of a related disorder. In some embodiments, prevention is assessed on a population basis such that an agent is considered to “prevent” a particular disease, disorder or condition if a statistically significant decrease in the development, frequency or intensity of one or more symptoms of the disease, disorder or condition is observed in a population susceptible to the disease, disorder or condition. Prevention may be considered complete when onset of disease, disorder or condition has been delayed for a predefined period of time.

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development. In some embodiments, a subject is a patient with one or more diseases or conditions selected from the group consisting of atopic dermatitis, atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, Crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa.

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and
(3) ameliorating the disease; for example, ameliorating a disease, condition or disorder in an individual that is experiencing or displaying the pathology or symptomatology of the disease, condition or disorder (i.e., reversing the pathology or symptomatology).

For therapeutic use, beneficial or desired results include clinical results such as reducing incidence or amelioration of one or more symptoms of cancer in a patient.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

### Antibodies to IL-33

The disclosure provides antibodies that bind to IL-33. IL-33 antibodies may bind one or more additional targets. IL-33 is also known as 9orf26, DVS27, IL1F11, NF-HEV, NFEHEV, and IL-1F11. As used herein, the term “IL-33” includes variants, isoforms, homologs, orthologs and paralogs of IL-33. In some embodiments, an antibody disclosed herein cross-reacts with IL-33 from species other than human, such as IL-33 of cynomolgus monkey, as well as different forms of IL-33. In some embodiments, an antibody may be completely specific for human IL-33 and may not exhibit species cross-reactivity or other types of cross-reactivity. As used herein the term IL-33 refers to naturally occurring human IL-33 unless contextually dictated otherwise. An “IL-33 antibody” “anti-IL-33 antibody” or other similar designation means any antibody (as defined herein) that binds or reacts with IL-33, an isoform, fragment or derivative thereof. The full length, mature form of IL-33, is represented by UniProtKB/Swiss-Prot accession number 095760. The full length, mature form of murine IL-33, is represented by UniProtKB/Swiss-Prot accession number Q8BVZ5. In some aspects, IL-33 antibodies of the invention specifically bind residues 112-270 of human IL-33.

In some embodiments, the invention provides an IL-33 antibody having a light chain variable region (VL) sequence and a heavy chain variable region (VH) sequence as found in one or more of Table 82, 84, 85, 86, and 87, or variants thereof.

The invention also provides CDR portions of antibodies to IL-33. Determination of CDR regions is defined in Example 1. In some embodiments, the antibody comprises three CDRs of any one of the heavy chain variable regions shown in one or more of Table 82, 84, 85, 86, and 87. In some embodiments, the antibody comprises three CDRs of any one of the light chain variable regions shown one or more of Table 82, 84, 85, 86, and 87. In some embodiments, the antibody comprises three CDRs of any one of the heavy chain variable regions and three CDRs of any one of the light chain variable regions each shown in one or more of Table 82, 84, 85, 86, and 87.

In some embodiments, the antibody comprises the six CDRs of an IL-33 antibody selected from one or more of Table 82, 84, 85, 86, and 87. In some embodiments, the antibody comprises the VH and VL of an IL-33 antibody selected from one or more of Table 82, 84, 85, 86, and 87. In some embodiments, the antibody comprises the HC and LC of an IL-33 antibody selected from one or more of Table 82, 84, 85, 86, and 87.

In some embodiments, the disclosure provides anti-IL-33 antibodies containing variations of the CDRs, VH, VL, HC, and LC regions shown in one or more of Table 82, 84, 85, 86, and 87, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 82, 84, 85, 86, and 87. These amounts are not meant to be limiting and increments between the recited percentages are specifically envisioned as part of the disclosure.

In some aspects, the disclosure provides an isolated antibody that specifically binds to IL-33, comprising a heavy chain variable region (IL33-VH) and a light chain variable region (IL33-VL), comprising
(i) the CDR-H1, CDR-H2, and CDR-H3 sequences of SEQ ID NO: 63, and the CDR-L1, CDR-L2, and CDR-L3 sequences of SEQ ID NO: 71;
(ii) the CDR-H1, CDR-H2, and CDR-H3 sequences of SEQ ID NO: 80, and the CDR-L1, CDR-L2, and CDR-L3 sequences of SEQ ID NO: 81;
(iii) the CDR-H1, CDR-H2, and CDR-H3 sequences of SEQ ID NO: 73, and the CDR-L1, CDR-L2, and CDR-L3 sequences of SEQ ID NO: 78;

In some aspects, the disclosure provides an isolated antibody that specifically binds to IL-33, comprising a heavy chain variable region (IL33-VH) and a light chain variable region (IL33-VL), comprising the CDR-H1, CDR-H2, and CDR-H3 sequences of SEQ ID NO: 73, and the CDR-L1, CDR-L2, and CDR-L3 sequences of SEQ ID NO: 78.

In some aspects, the disclosure provides an isolated antibody that specifically binds to IL-33, comprising a heavy chain variable region (IL33-VH) and a light chain variable region (IL33-VL), wherein the CDR-H1 comprises the amino acid sequence of SEQ ID NO: 60; the CDR-H2 comprises the amino acid sequence of SEQ ID NO: 61; the CDR-H3 comprises the amino acid sequence of SEQ ID NO: 72; the CDR-L1 comprises the amino acid sequence of SEQ ID NO: 75; the CDR-L2 comprises the amino acid sequence of SEQ ID NO: 76, and the CDR-L3 comprises the amino acid sequence of SEQ ID NO: 77.

The IL-33 antibody may comprise an IL33-VH framework sequence comprising a human germline VH framework sequence. The IL33-VH framework sequence may comprise one or more amino acid substitutions, additions, or deletions, while still retaining functional and structural similarity with the germline from which it was derived. In some aspects, the VH framework is at least 90%, at least 91%, at least 92%, at least 93%, at least 94%, at least 95%, at least 96%, at least 97%, at least 98%, at least 99%, or 100% identical to a human germline VH framework sequence. In some aspects, the IL-33 antibody comprises an IL33-VH framework sequence comprising 1, 2, 3, 4, 5, 6, 7, 8, 9, 10 amino acid substitutions, additions or deletions relative to the human germline VH framework sequence. In some aspects, the 1, 2, 3, 4, 5, 6, 7, 8, 9, or 10 amino acid substitutions, additions or deletions are only in the framework regions. In some aspects, the % identity is based on similarity with VH excluding those portions herein defined as CDRs.

In some aspects, the IL33-VH framework sequence may be derived from a human germline VH sequence selected from the group consisting of DP47, DP48, DP50, DP51, DP54, and DP77. In some aspects, the IL33-VH framework sequence is derived from DP54. The IL33-VH framework sequence may be derived from a human germline VH sequence selected from the group consisting of IGHV3-7*01/3-7*02/3-7*03, IGHV3-23*01/3-23D*01, IGHV3-23*04/3-23D*02, IGHV3-23*02, IGHV3-64*04, IGHV3-13*01/3-13*04, IGHV3-48*01/3-48*04, IGHV3-48*03, IGHV3-48*02, IGHV3-13*05, IGHV3-21*01/3-21*02/3-21*03/3-21*04, and IGHV3-33*01/3-33*04. In some aspects, the IL33-VH framework sequence is derived from IGHV3-7*01/3-7*02/3-7*03.

The invention has identified the human germline VH framework IGHV3-7*01/3-7*02/3-7*03 (DP-54) as highly advantageous with IL33-VH CDRs of the invention. Advantageously, other germlines may also be used with IL33-CDRs of the invention, such as IGHV3-23*01/3-23D*01 (DP-47) and other IGHV3-23 loci germline including IGHV3-23*04/3-23D*02 and IGHV3-23*02, IGHV3-64*04, IGHV3-13*01/3-13*04 (DP-48), IGHV3-48*01/3-48*04, IGHV3-48*03, IGHV3-48*02 (DP-51), IGHV3-13*05, IGHV3-21*01/3-21*02/3-21*03/3-21*04 (DP-77), and IGHV3-33*01/3-33*04 (DP-50). The foregoing frameworks are modelled to be compatible with IL33-VH CDRs of the invention.

The IL-33 antibody may comprise an IL33-VL framework sequence comprising a human germline VL framework sequence. The IL33-VL framework sequence may comprise one or more amino acid substitutions, additions, or deletions, while still retaining functional and structural similarity with the germline from which it was derived. In some aspects, the VL framework is at least 90%, at least 91%, at least 92%, at least 93%, at least 94%, at least 95%, at least 96%, at least 97%, at least 98%, at least 99%, or 100% identical to a human germline VL framework sequence. In some aspects, the IL-33 antibody comprises an IL33-VL framework sequence comprising 1, 2, 3, 4, 5, 6, 7, 8, 9, 10 amino acid substitutions, additions or deletions relative to the human germline VL framework sequence. In some aspects, the 1, 2, 3, 4, 5, 6, 7, 8, 9, or 10 amino acid substitutions, additions or deletions are only in the framework regions. In some aspects, the % identity is based on similarity with VL excluding those portions herein defined as CDRs.

In some aspects, the IL33-VL framework sequence may be derived from a human germline VL sequence selected from the group consisting of DPK1, DPK3, DPK4, DPK5, DPK7, DPK8, and DPK9. In some aspects, the IL33-VL framework sequence is derived from DPK9. The IL33-VL framework sequence may be derived from a human germline VL sequence selected from the group consisting of IGKV1-39*01/1D-39*01, IGKV1-16*01, IGKV1-16*02, IGKV1-27*01, IGKV1-NL1*01, IGKV1-17*01, IGKV1-17, IGKV1-17*02, IGKV1-17*03, IGKV1-33*01/1D-33*01, IGKV1-6*01/1-6*02, IGKV1-12*01/1-12*02/1D-12*01/1D-12*02, IGKV1-5*03, IGKV1-5, IGKV1-5*01, IGKV1-5*02, IGKV1D-16*01, and IGKV1-9*01. In some aspects, the IL33-VL framework sequence is derived from IGKV1-39*01/1 D-39*01.

The invention has identified the human germline VL framework IGKV1-39*01/1 D-39*01 (DPK9) as highly advantageous with IL33-VL CDRs of the invention. Other alternate germlines for grafting to IL33-VL CDRs include: IGKV1-16*01 and other IGKV1-16 loci germlines including IGKV1-16*02, IGKV1-27*01 (DPK4), IGKV1-NL1*01, IGKV1-17*01 and other IGKV1-17 germline loci including IGKV1-17*02 and IGKV1-17*03, IGKV1-33*01/1D-33*01 (DPK1), IGKV1-6*01/1-6*02 (DPK3), IGKV1-12*01/1-12*02/1 D-12*01/1 D-12*02 (DPK5), IGKV1-5*03 and other IGKV1-5 loci germlines including IGKV1-5*01 and IGKV1-5*02, IGKV1D-16*01 (DPK7), IGKV1-9*01 (DPK8). The foregoing frameworks are modelled to be compatible with IL33-VL CDRs of the invention.

In some aspects of the disclosure, the IL33-CH1 of the antibody comprises a sequence selected from the group consisting of SEQ ID NO: 6, SEQ ID NO: 105, and SEQ ID NO: 110. In some aspects of the disclosure, the IL33-CL of the antibody comprises a sequence selected from the group consisting SEQ ID NO: 16, SEQ ID NO: 108, and SEQ ID NO: 113. The IL33-CL may comprise a sequence according to SEQ ID NO:16. The IL33-CH1 may comprise a sequence according to SEQ ID NO: 6. The IL33-CH1 and IL33-CL may each be part of a multispecific antibody.

The IL33-CH1 may be connected to the IL33-VL, and the IL33-CL may be connected to the IL33-VH forming an IL-33-binding domain-swap Fab domain (IL33-xFab). Domain-swap Fabs are depicted in FIGS. 18 D, G, and H. Alternatively, the IL33-CH1 may be connected to the IL33-VH, and the IL33-CL may be connected to the IL33-VL forming an IL-33 binding Fab domain (IL33-Fab). A Fab domain is depicted in FIGS. 18 A, B, C, E, F, and I. IL-33 antibodies of the invention may comprise a hinge region. The hinge region may be selected from any suitable sequence, including a sequence selected from any of Tables 82, 85, and 87. In some aspects, the hinge region is selected from the group consisting of SEQ ID NO: 7, SEQ ID NO: 102, SEQ ID NO: 123, SEQ ID NO:126, SEQ ID NO: 129, and SEQ ID NO:131.

The IL33-CL may be connected to a hinge region which is then connected to a CH2 domain. Alternatively, the IL33-CH1 may be connected to a hinge region which is then connected to a CH2 domain. The CH2 region may comprise a sequence selected from any one of Tables 80, 81, 82, 83, 85, and 87. The CH2 domain may comprise SEQ ID NO: 8. The CH2 region may be connected to a CH3 region. The CH3 region may comprise a sequence selected from any one of Tables 80, 81, 82, 83, 85, and 87. The CH3 region may comprise a sequence selected from the group consisting of SEQ ID NO: 9, SEQ ID NO: 106, SEQ ID NO: 111, SEQ ID NO: 114, SEQ ID NO: 117, SEQ ID NO: 124, SEQ ID NO: 127, SEQ ID NO: 139, SEQ ID NO: 141, SEQ ID NO: 147, and SEQ ID NO: 148.

The IL33-HC may comprise a sequence selected from any one of Tables 82, 85, and 87. The IL33-HC may comprise a sequence at least 90%, 91%, 92%, 93%, 94%, 95%, 96%, 97%, 98%, 99%, or 100% identical to a sequence selected from the group consisting of SEQ ID NO: 74, SEQ ID NO:103, SEQ ID NO: 128, SEQ ID NO: 132, SEQ ID NO: 134, SEQ ID NO: 137, SEQ ID NO: 142, and SEQ ID NO: 143.

The IL33-VL bearing polypeptide may comprise a sequence selected from any one of Tables 82, 85, and 87. The IL33-VL bearing polypeptide may comprise a sequence at least 90%, 91%, 92%, 93%, 94%, 95%, 96%, 97%, 98%, 99%, or 100% identical to a sequence selected from the group consisting of SEQ ID NO: 79 SEQ ID NO: 107, SEQ ID NO: 115, SEQ ID NO: 121, and SEQ ID NO: 138, SEQ ID NO: 144, and SEQ ID NO: 145.

IL-33 antibodies of the disclosure advantageously bind to both human and cynomolgus monkey within one order of magnitude or less. This facilitates using animal and toxicology data to inform human dosing. The IL-33 antibodies of the invention advantageously have improved binding affinity and IL-33 neutralization compared to the parental antibody.

The IL-33 antibodies of the present disclosure demonstrate reduced deamidation liabilities at CDRL1 residue 28. Preferentially, the reduced deamidation is achieved by mutation removal of N28 and replacing with alternative residue. In some aspects, the IL-33 antibody comprises a CDRL1 that does not comprise an Asn residue at position 28. In some aspects, the IL-33 antibody comprises a Pro residue at position 28 in CDRL1.

The IL-33 antibodies of the present disclosure demonstrate reduced deamidation liabilities at CDRL1 residue 30. Preferentially, the reduced deamidation is achieved by mutation removal of N30 and replacing with alternative residue. In some aspects, the IL-33 antibody comprises a CDRL1 that does not comprise an Asn residue at position 30. In some aspects, the IL-33 antibody comprises a His residue at position 28 in CDRL1.

In some aspects, the IL-33 antibody comprises improved binding affinity over the parental antibody. The improvement may be at least 10-fold better binding. The IL-33 antibody of the invention may bind to IL-33 with an affinity of less than 1 pM. The IL-33 antibody of the invention may bind to IL-33 with an affinity of less than 500 fM. The IL-33 antibody of the invention may bind to IL-33 with an affinity of less than 250 fM. The binding affinity may be determined by Kinetics Exclusion Assay (KinExA). The KinExA may be analysed with KinExA Pro Software version 4.3.1.1 from Sapidyne.

In some aspects, the disclosure provides an isolated antibody that specifically binds IL-33 comprising a heavy chain variable region (IL33-VH) and a light chain variable region (IL33-VL), wherein the IL33-VH comprises the CDR-H1, CDR-H2, and CDR-H3 sequences of encoded by the plasmid deposited at the ATCC and having ATCC Accession No. PTA-127210, and the IL33-VL comprises the CDR-L1, CDR-L2, and CDR-L3 sequences encoded by the plasmid deposited at the ATCC and having ATCC Accession No. PTA-127209.

In some aspects, the disclosure provides an isolated antibody that specifically binds IL-33 comprising an IL33-VH sequence encoded by the plasmid deposited at the ATCC and having ATCC Accession No. PTA-127210. In some aspects, the disclosure provides an isolated antibody that specifically binds IL-33 comprising an IL33-VL sequence encoded by the plasmid deposited at the ATCC and having ATCC Accession No. PTA-127209. In some aspects, the disclosure provides an isolated antibody comprising an IL33-VH bearing polypeptide sequence encoded by the plasmid deposited at the ATCC and having ATCC Accession No. PTA-127208. In some aspects, the disclosure provides an isolated antibody comprising an IL33-VL bearing polypeptide sequence encoded by the plasmid deposited at the ATCC and having ATCC Accession No. PTA-127207.

### Antibodies to TSLP

The disclosure provides antibodies that bind to TSLP. TSLP antibodies may bind one or more additional targets. TSLP is also known as thymic stromal lymphoprotein. As used herein, the term “TSLP” includes variants, isoforms, homologs, orthologs and paralogs of one or more of TSLP. In some embodiments, an antibody disclosed herein cross-reacts with TSLP from species other than human, such as TSLP of cynomolgus monkey, as well as different forms of TSLP. In some embodiments, an antibody may be completely specific for human TSLP and may not exhibit species cross-reactivity or other types of cross-reactivity. As used herein the term TSLP refers to naturally occurring human TSLP unless contextually dictated otherwise. A “TSLP antibody” “anti-TSLP antibody” or other similar designation means any antibody (as defined herein) that binds or reacts with TSLP, an isoform, fragment or derivative thereof. The full length, mature form of TSLP, is represented by UniProtKB/Swiss-Prot accession number Q969D9. The full length, mature form of mouse TSLP, is represented by UniProtKB/Swiss-Prot accession number Q9JIE6.

In some embodiments, the invention provides a TSLP antibody having a light chain variable region (VL) sequence and a heavy chain variable region (VH) sequence as found in one or more of Table 83, 84, 85, 86, and 87, or variants thereof.

The invention also provides CDR portions of antibodies to TSLP. Determination of CDR regions is defined in Example 1. In some embodiments, the antibody comprises three CDRs of any one of the heavy chain variable regions shown in one or more of Table 83, 84, 85, 86, and 87. In some embodiments, the antibody comprises three CDRs of any one of the light chain variable regions shown in one or more of Table 83, 84, 85, 86, and 87. In some embodiments, the antibody comprises three CDRs of any one of the heavy chain variable regions and three CDRs of any one of the light chain variable regions each shown in one or more of Table 83, 84, 85, 86, and 87.

In some embodiments, the antibody comprises the six CDRs of a TSLP antibody selected from one or more of Table 83, 84, 85, 86, and 87. In some embodiments, the antibody comprises the VH and VL of a TSLP antibody each selected from one or more of Table 83, 84, 85, 86, and 87. In some embodiments, the antibody comprises the HC and LC of an TSLP antibody each selected from one or more of Table 83, 84, 85, 86, and 87.

In some embodiments, the disclosure provides anti-TSLP antibodies containing variations of the CDRs, VH, VL, HC, and LC regions shown in one or more of Table 83, 84, 85, 86, and 87, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 83, 84, 85, 86, and 87. These amounts are not meant to be limiting and increments between the recited percentages are specifically envisioned as part of the disclosure.

In some aspects, the disclosure provides an isolated antibody that specifically binds to TSLP, comprising a heavy chain variable region (TSLP-VH) and a light chain variable region (TSLP-VL), comprising
(i) the CDR-H1, CDR-H2, and CDR-H3 sequences of SEQ ID NO: 92, and the CDR-11, CDR-L2, and CDR-L3 sequences of SEQ ID NO: 93;
(ii) the CDR-H1, CDR-H2, and CDR-H3 sequences of SEQ ID NO: 92, and the CDR-11, CDR-L2, and CDR-L3 sequences of SEQ ID NO: 94;
(iii) the CDR-H1, CDR-H2, and CDR-H3 sequences of SEQ ID NO: 92, and the CDR-11, CDR-L2, and CDR-L3 sequences of SEQ ID NO: 213;
(iv) the CDR-H1, CDR-H2, and CDR-H3 sequences of SEQ ID NO: 92, and the CDR-11, CDR-L2, and CDR-L3 sequences of SEQ ID NO: 214;
(v) the CDR-H1, CDR-H2, and CDR-H3 sequences of SEQ ID NO: 221, and the CDR-11, CDR-L2, and CDR-L3 sequences of SEQ ID NO: 213;
(vi) the CDR-H1, CDR-H2, and CDR-H3 sequences of SEQ ID NO: 221, and the CDR-11, CDR-L2, and CDR-L3 sequences of SEQ ID NO: 94; or
(vii) the CDR-H1, CDR-H2, and CDR-H3 sequences of SEQ ID NO: 221, and the CDR-11, CDR-L2, and CDR-L3 sequences of SEQ ID NO: 223.

In some aspects, the disclosure provides an isolated antibody that specifically binds to TSLP, comprising a heavy chain variable region (TSLP-VH) and a light chain variable region (TSLP-VL), comprising the CDR-H1, CDR-H2, and CDR-H3 sequences of SEQ ID NO: 92, and the CDR-L1, CDR-L2, and CDR-L3 sequences of SEQ ID NO: 94.

In some aspects, the disclosure provides an isolated antibody that specifically binds to TSLP, comprising a heavy chain variable region (TSLP-VH) and a light chain variable region (TSLP-VL), comprising the CDR-H1, CDR-H2, and CDR-H3 sequences of SEQ ID NO: 92, and the CDR-L1, CDR-L2, and CDR-L3 sequences of SEQ ID NO: 213.

In some aspects, the disclosure provides an isolated antibody that specifically binds to TSLP, comprising a heavy chain variable region (TSLP-VH) and a light chain variable region (TSLP-VL), comprising the CDR-H1, CDR-H2, and CDR-H3 sequences of SEQ ID NO: 92, and the CDR-L1, CDR-L2, and CDR-L3 sequences of SEQ ID NO: 214.

In some aspects, the disclosure provides an isolated antibody that specifically binds to TSLP, comprising a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 90.

In some aspects, the disclosure provides an isolated antibody that specifically binds to TSLP, comprising a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 211.

In some aspects, the disclosure provides an isolated antibody that specifically binds to TSLP, comprising a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 212.

In some aspects, the disclosure provides an isolated antibody that specifically binds to TSLP, comprising a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 211.

In some aspects, the disclosure provides an isolated antibody that specifically binds to TSLP, comprising a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 90.

In some aspects, the disclosure provides an isolated antibody that specifically binds to TSLP, comprising a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 212.

The TSLP antibody may comprise an TSLP-VH framework sequence comprising a human germline VH framework sequence. The TSLP-VH framework sequence may comprise one or more amino acid substitutions, additions, or deletions, while still retaining functional and structural similarity with the germline from which it was derived. In some aspects, the VH framework is at least 90%, at least 91%, at least 92%, at least 93%, at least 94%, at least 95%, at least 96%, at least 97%, at least 98%, at least 99%, or 100% identical to a human germline VH framework sequence. In some aspects, the TSLP antibody comprises an TSLP-VH framework sequence comprising 1, 2, 3, 4, 5, 6, 7, 8, 9, 10 amino acid substitutions, additions or deletions relative to the human germline VH framework sequence. In some aspects, the 1, 2, 3, 4, 5, 6, 7, 8, 9, or 10 amino acid substitutions, additions or deletions are only in the framework regions. In some aspects, the % identity is based on similarity with VH excluding those portions herein defined as CDRs.

In some aspects, the TSLP-VH framework sequence may be derived from a human germline VH sequence selected from the group consisting of DP47, DP49, DP50, DP54, and DP53. In some aspects, the TSLP-VH framework sequence is derived from DP50.

The TSLP-VH framework sequence may be derived from a human germline VH sequence selected from the group consisting of IGHV3-33*01, IGHV3-7*01, IGHV3-33*02, IGHV3-30*03, IGHV3-30*04, IGHV3-30*01, IGHV3-23*01, IGHV3-23*03, IGHV3-74*01, and IGHV3-74*03. In some aspects, the TSLP-VH framework sequence is derived from IGHV3-33*01.

The invention has identified the human germline VH framework IGHV3-33*01 (DP-50) as highly advantageous for TSLP-VH CDRs of the invention. TSLP-0875 V_{H} has one framework difference from DP-50: V(H2)M. The parent antibody also has one framework difference from DP-50: V(H2)M. The inventors have confirmed in experimental testing that the V_{H} CDR graft onto IGHV3-7*01 (DP-54) with V(H2)M framework back-mutation is fully active. Other germlines that are modelled to be advantageous for TSLP V_{H} CDR grafting include IGHV3-33*02, IGHV3-30*03 (DP-49), IGHV3-30*04 and other IGHV3-30 loci germlines including IGHV3-30*01, IGHV3-23*01 (DP-47) and other IGHV3-23 loci germlines including IGHV3-23*03, IGHV3-74*01 (DP-53) and other IGHV3-74 loci germlines including IGHV3-74*03. The foregoing frameworks are modelled to be compatible with TSLP-VH CDRs of the invention.

The TSLP antibody may comprise an TSLP-VL framework sequence comprising a human germline VL framework sequence. The TSLP-VL framework sequence may comprise one or more amino acid substitutions, additions, or deletions, while still retaining functional and structural similarity with the germline from which it was derived. In some aspects, the VL framework is at least 90%, at least 91%, at least 92%, at least 93%, at least 94%, at least 95%, at least 96%, at least 97%, at least 98%, at least 99%, or 100% identical to a human germline VL framework sequence. In some aspects, the TSLP antibody comprises an TSLP-VL framework sequence comprising 1, 2, 3, 4, 5, 6, 7, 8, 9, 10 amino acid substitutions, additions or deletions relative to the human germline VL framework sequence. In some aspects, the 1, 2, 3, 4, 5, 6, 7, 8, 9, or 10 amino acid substitutions, additions or deletions are only in the framework regions. In some aspects, the % identity is based on similarity with VL excluding those portions herein defined as CDRs.

In some aspects, the TSLP-VL framework sequence may be derived from a human germline VL sequence selected from the group consisting of DPK1, DPK3, DPK4, DPK5, DPK7, DPK8, DPK9, and DPK24. In some aspects, the TSLP-VL framework sequence is derived from DPK9.

The TSLP-VL framework sequence may be derived from a human germline VL sequence selected from the group consisting of IGKV1-39*01, IGKV4-1*01, 1D-39*01, IGKV1-12*01, IGKV1-9*01, IGKV1-16*01, IGKV1-16*02, IGKV1-33*01/1D-33*01, IGKV1-27*01, IGKV1D-16*01, IGKV1-13*02/1D-13*02, IGKV1-17*01, IGKV1-17*02, IGKV1-17*03, and IGKV1-6*01/1-6*02. In some aspects, the TSLP-VL framework sequence is derived from IGKV1-39*01.

The invention has identified the human germline VL framework DPK9 (IGKV1-39*01) as highly advantageous with TSLP-VL CDRs of the invention. IGKV4-1*01 (DPK24) is also predicted to be highly advantageous, as this functions well with GSK 3B9 VL. Advantageously, other VH germlines that may be used with IL4-VH regions of the invention include the group consisting of 1D-39*01, IGKV1-12*01 (DPK5), IGKV1-9*01 (DPK8), IGKV1-16*01, IGKV1-16*02, IGKV1-33*01/1 D-33*01 (DPK1), IGKV1-27*01 (DPK4), IGKV1 D-16*01 (DPK7), IGKV1-13*02/1D-13*02, IGKV1-17*01, IGKV1-17*02, IGKV1-17*03, and IGKV1-6*01/1-6*02 (DPK3). The foregoing frameworks are modelled to be compatible with TSLP-VL CDRs of the invention.

In some aspects of the disclosure, the TSLP-CH1 of the antibody comprises a sequence selected from the group consisting of SEQ ID NO: 6, SEQ ID NO: 105, and SEQ ID NO: 110. In some aspects of the disclosure, the TSLP-CL of the antibody comprises a sequence selected from the group consisting SEQ ID NO: 16, SEQ ID NO: 95, SEQ ID NO: 108, and SEQ ID NO: 113. The TSLP-CH1 may comprise a sequence according to SEQ ID NO: 6. The TSLP-CL may comprise a sequence according to SEQ ID NO: 95. The TSLP-CH1 and TSLP-CL may each be part of a multispecific antibody.

The TSLP-CH1 may be connected to the TSLP-VL, and the TSLP-CL may be connected to the TSLP-VH forming a TSLP-binding domain-swap Fab domain (TSLP-xFab). Domain-swap Fabs are depicted in FIGS. 18 D, G, and H. Alternatively, the TSLP-CH1 may be connected to the TSLP-VH, and the TSLP-CL may be connected to the TSLP-VL forming a TSLP-binding Fab domain (TSLP-Fab). A Fab domain is depicted in FIGS. 18 A, B, C, E, F, and I.

TSLP antibodies of the invention may comprise a hinge region. The hinge region may be selected from any suitable sequence, including a sequence selected from any of Tables 82, 85, and 87. In some aspects, the hinge region is selected from the group consisting of SEQ ID NO: 7, SEQ ID NO: 102, SEQ ID NO: 123, SEQ ID NO:126, SEQ ID NO: 129, and SEQ ID NO:131.

The TSLP-CL may be connected to a hinge region which is then connected to a CH2 domain. Alternatively, the TSLP-CH1 may be connected to a hinge region which is then connected to a CH2 domain. The CH2 region may comprise a sequence selected from any one of Tables 80, 81, 82, 83, 85, and 87. The CH2 domain may comprise SEQ ID NO: 8. The CH2 region may be connected to a CH3 region. The CH3 region may comprise a sequence selected from any one of Tables 80, 81, 82, 83, 85, and 87. The CH3 region may comprise a sequence selected from the group consisting of SEQ ID NO: 9, SEQ ID NO: 106, SEQ ID NO: 111, SEQ ID NO: 114, SEQ ID NO: 117, SEQ ID NO: 124, SEQ ID NO: 127, SEQ ID NO: 139, SEQ ID NO: 141, SEQ ID NO: 147, and SEQ ID NO: 148.

The TSLP-VH bearing polypeptide may comprise a sequence selected from any one of Tables 83, 84, and 87. The TSLP-VH bearing polypeptide may comprise a sequence at least 90%, 91%, 92%, 93%, 94%, 95%, 96%, 97%, 98%, 99%, 100% identical to a sequence selected from the group consisting of SEQ ID NO: 97, SEQ ID NO: 152, SEQ ID NO: 153, SEQ ID NO: 155, SEQ ID NO: 158, SEQ ID NO: 161, SEQ ID NO:165, and SEQ ID NO: 222.

The TSLP-VL bearing polypeptide may comprise a sequence selected from any one of Tables 83, 84, and 87. The TSLP-VL bearing polypeptide may comprise a sequence at least 90%, 91%, 92%, 93%, 94%, 95%, 96%, 97%, 98%, 99%, or 100% identical to a sequence selected from the group consisting of SEQ ID NO: 98, SEQ ID NO: 99, SEQ ID NO: 150, SEQ ID NO: 154, SEQ ID NO: 156, SEQ ID NO: 157, SEQ ID NO: 160, SEQ ID NO: 215, SEQ ID NO: 216, and SEQ ID NO: 224.

TSLP antibodies of the disclosure advantageously bind to both human and cynomolgus monkey within one order of magnitude or less. This facilitates using animal and toxicology data to provide inform human dosing. The TSLP antibodies of the invention advantageously have improved binding affinity and TSLP neutralization compared to the parental antibody.

TSLP antibodies of the present disclosure demonstrate improved anti-TSLP bioactivity as measured in a TARC production bioassay in human peripheral blood monocytes. TSLP antibodies demonstrate anti-TSLP bioactivity of an IC₅₀ of less than 10 pM as measured in a TARC production bioassay in human peripheral blood monocytes. TSLP antibodies demonstrate anti-TSLP bioactivity of an IC₅₀ of less than 6 pM as measured in a TARC production bioassay in human peripheral blood monocytes.

The TSLP antibodies of the present disclosure demonstrate a combination of improved anti-TSLP bioactivity while minimizing an increase in viscosity. TSLP antibodies of the present disclosure have a viscosity of 20 cP at concentrations of at least 100 mg/mL. TSLP antibodies of the present disclosure have a viscosity of 20 cP at concentrations of at least 110 mg/mL. TSLP antibodies of the present disclosure have a viscosity of 20 cP at concentrations of at least 120 mg/mL.

In some aspects, the disclosure provides an isolated antibody that specifically binds TSLP comprising a heavy chain variable region (TSLP-VH) and a light chain variable region (TSLP-VL), wherein the TSLP-VH comprises the CDR-H1, CDR-H2, and CDR-H3 sequences of encoded by the plasmid deposited at the ATCC and having ATCC Accession No. PTA-127200, and the TSLP-VL comprises the CDR-L1, CDR-L2, and CDR-L3 sequences encoded by the plasmid deposited at the ATCC and having ATCC Accession No. PTA-127199.

In some aspects, the disclosure provides an isolated antibody that specifically binds TSLP comprising a heavy chain variable region (TSLP-VH) and a light chain variable region (TSLP-VL), wherein the TSLP-VH comprises the CDR-H1, CDR-H2, and CDR-H3 sequences of encoded by the plasmid deposited at the ATCC and having ATCC Accession No. PTA-127200, and the TSLP-VL comprises the CDR-L1, CDR-L2, and CDR-L3 sequences encoded by the plasmid deposited at the ATCC and having ATCC Accession No. PTA- - - - .

In some aspects, the disclosure provides an isolated antibody that specifically binds TSLP comprising a heavy chain variable region (TSLP-VH) and a light chain variable region (TSLP-VL), wherein the TSLP-VH comprises the CDR-H1, CDR-H2, and CDR-H3 sequences of encoded by the plasmid deposited at the ATCC and having ATCC Accession No. PTA-127200, and the TSLP-VL comprises the CDR-L1, CDR-L2, and CDR-L3 sequences encoded by the plasmid deposited at the ATCC and having ATCC Accession No. PTA- - - - .

In some aspects, the disclosure provides an isolated antibody comprising the TSLP-VL sequence encoded by the plasmid deposited at the ATCC and having ATCC Accession No. PTA-127199. In some aspects, the disclosure provides an isolated antibody comprising the TSLP-VH sequence encoded by the plasmid deposited at the ATCC and having ATCC Accession No. PTA-127200. In some aspects, the disclosure provides an isolated antibody comprising the TSLP-VH bearing polypeptide sequence encoded by the plasmid deposited at the ATCC and having ATCC Accession No. PTA-127202. In some aspects, the disclosure provides an isolated antibody comprising the TSLP-VL bearing polypeptide sequence encoded by the plasmid deposited at the ATCC and having ATCC Accession No. PTA-127201.

In some aspects, the disclosure provides an isolated antibody comprising the TSLP-VL sequence encoded by the plasmid deposited at the ATCC and having ATCC Accession No. PTA- - - - . In some aspects, the disclosure provides an isolated antibody comprising the TSLP-VL bearing polypeptide sequence encoded by the plasmid deposited at the ATCC and having ATCC Accession No. PTA- - - - .

In some aspects, the disclosure provides an isolated antibody comprising the TSLP-VL sequence encoded by the plasmid deposited at the ATCC and having ATCC Accession No. PTA- - - - . In some aspects, the disclosure provides an isolated antibody comprising the TSLP-VH sequence encoded by the plasmid deposited at the ATCC and having ATCC Accession No. PTA-127200.

In some aspects, the disclosure provides an isolated antibody comprising the TSLP-VL sequence encoded by the plasmid deposited at the ATCC and having ATCC Accession No. PTA- - - - . In some aspects, the disclosure provides an isolated antibody comprising the TSLP-VH sequence encoded by the plasmid deposited at the ATCC and having ATCC Accession No. PTA-127200.

### Antibodies to p40

The disclosure provides antibodies that bind to p40. Anti-p40 antibodies of the invention may bind one or more additional targets. P40 is also known as IL12B, CLMF, CLMF2, IL-12B, IMD28, NKSF, NKSF2, and IMD29. As used herein, the term “p40” includes variants, isoforms, homologs, orthologs and paralogs of one or more of p40. In some embodiments, an antibody disclosed herein cross-reacts with p40 from species other than human, such as p40 of cynomolgus monkey, as well as different forms of p40. In some embodiments, an antibody may be completely specific for human p40 and may not exhibit species cross-reactivity or other types of cross-reactivity. As used herein the term p40 refers to naturally occurring human p40 unless contextually dictated otherwise. A “p40 antibody” “anti- p40 antibody” or other similar designation means any antibody (as defined herein) that binds or reacts with p40, an isoform, fragment or derivative thereof. The full length, mature form of p40 is represented by UniProtKB/Swiss-Prot accession number P29460. The full length, mature form of cynomolgus monkey p40, is represented by UniProtKB/Swiss-Prot accession number G7P6S2.

The p40 antibody may comprise a p40-VH framework sequence comprising a human germline VH framework sequence. The p40-VH framework sequence may comprise one or more amino acid substitutions, additions, or deletions, while still retaining functional and structural similarity with the germline from which it was derived. In some aspects, the VH framework sequence is at least 90%, at least 91%, at least 92%, at least 93%, at least 94%, at least 95%, at least 96%, at least 97%, at least 98%, at least 99%, or 100% identical to a human germline VH framework sequence. In some aspects, the p40 antibody comprises an p40-VH framework sequence comprising 1, 2, 3, 4, 5, 6, 7, 8, 9, 10 amino acid substitutions, additions or deletions relative to the human germline VH framework sequence. In some aspects, the 1, 2, 3, 4, 5, 6, 7, 8, 9, or 10 amino acid substitutions, additions or deletions are only in the framework regions. In some aspects, the % identity is based on similarity with VH excluding those portions herein defined as CDRs.

The disclosure provides for an isolated antibody that specifically binds to p40 through a p40 binding domain and wherein the antibody comprises at least one additional antigen binding domain that specifically binds to an antigen selected from the group consisting of IL-4, IL-13, IL-33, and TSLP, wherein the p40 binding domain comprises a heavy chain variable region (p40-VH) and a light chain variable region (p40-VL), comprising the CDR-H1, CDR-H2, and CDR-H3 sequence of SEQ ID NO: 169, and the CDR-L1, CDR-L2, and CDR-L3 sequences of SEQ ID NO: 175.

The disclosure provides for an isolated antibody that specifically binds to p40 through a p40 binding domain and wherein the antibody comprises at least one additional antigen binding domain that specifically binds to an antigen selected from the group consisting of IL-4, IL-13, IL-33, and TSLP, and wherein the p40 binding domain comprises a heavy chain variable region (p40-VH) and a light chain variable region (p40-VL), wherein the CDR-H1 of the p40 binding domain comprises the amino acid sequence of SEQ ID NO: 166; the CDR-H2 of the p40 binding domain comprises the amino acid sequence of SEQ ID NO: 167; the CDR-H3 of the p40 binding domain comprises the amino acid sequence of SEQ ID NO: 168; the CDR-L1 of the p40 binding domain comprises the amino acid sequence of SEQ ID NO: 171; the CDR-L2 of the p40 binding domain comprises the amino acid sequence of SEQ ID NO: 172, and the CDR-L3 of the p40 binding domain comprises the amino acid sequence of SEQ ID NO: 173.

The p40 antibody may also comprises one or both of an IL-4 binding domain that specifically binds to IL-4, and an IL-13 binding domain that specifically binds to IL-13.

In some aspects, the p40-VH framework sequence may be derived from a human germline VH sequence selected from the group consisting of DP3, DP7, DP73, DP75, and DP88. In some aspects, the TSLP-VH framework sequence is derived from DP73.

The p40-VH framework sequence may be derived from a human germline VH sequence selected from the group consisting of IGHV5-51*01/5-51*03, IGHV1-46*01/1-46*03, IGKV1-39*01, IGHV5-51*02, IGHV5-51*04, IGHV1-46*02, IGHV1-69-2*01, IGHV1-69*08, IGHV1-69*02, IGHV1-69*06/1-69*14, IGHV1-69*04/1-69*09 and IGHV1-2*02. In some aspects, the p40-VH framework sequence is derived from IGHV5-51*01/5-51*03.

The invention has identified the human germline to IGHV5-51*01/5-51*03 (DP-73) as highly advantageous for p40-VH CDRs of the invention. Experimental data demonstrates that IGHV1-46*01/1-46*03 (DP-7) and IGKV1-39*01 or DPK9 (with L46S mutation) for V_{L} and VK1-33 (with L46S mutation) were advantageously able to retain binding within ˜3-4 fold after grafting. Other possible germlines suitable for use with p40-VH CDRs of the invention include other IGHV5-51 loci germlines IGHV5-51*02 and IGHV5-51*04, other IGHV1-46 loci germlines such as IGHV1-46*02, IGHV1-69-2*01 (DP-3) and other IGHV1-69 loci germlines IGHV1-69*08, IGHV1-69*02, IGHV1-69*06/1-69*14 (DP-88), IGHV1-69*04/1-69*09 and IGHV1-2*02 (DP-75). The foregoing frameworks are modelled to be compatible with p40-VH CDRs of the invention.

The p40 antibody may comprise a p40-VL framework sequence comprising a human germline VL framework sequence. The p40-VL framework sequence may comprise one or more amino acid substitutions, additions, or deletions, while still retaining functional and structural similarity with the germline from which it was derived. In some aspects, the VL framework is at least 90%, at least 91%, at least 92%, at least 93%, at least 94%, at least 95%, at least 96%, at least 97%, at least 98%, at least 99%, or 100% identical to a human germline VL framework sequence. In some aspects, the p40 antibody comprises a p40-VL framework sequence comprising 1, 2, 3, 4, 5, 6, 7, 8, 9, 10 amino acid substitutions, additions or deletions relative to the human germline VL framework sequence. In some aspects, the 1, 2, 3, 4, 5, 6, 7, 8, 9, or 10 amino acid substitutions, additions or deletions are only in the framework regions. In some aspects, the % identity is based on similarity with VL excluding those portions herein defined as CDRs.

In some aspects, the p40-VL framework sequence may be derived from a human germline VL sequence selected from the group consisting of DPK4, DPK5, DPK7, DPK8, and DPK9. In some aspects, the p40-VL framework sequence is derived from DPK7.

The p40-VL framework sequence may be derived from a human germline VL sequence selected from the group consisting of IGKV1D-16*01, IGKV1D-16*02, IGKV1-16*01, IGKV1-16*02, IGKV1-39*01, IGKV1-12*01/1-12*02/1D-12*01/1D-12*02, IGKV1-9*01, IGKV1-5*03, IGKV1-5*01, and IGKV1-27*01. In some aspects, the p40-VL framework sequence is derived from IGKV1D-16*01.

The invention has identified the human germline VL framework IGKV1D-16*01 (DPK7) as highly advantageous with p40-VL CDRs of the invention. Advantageously, other VL germlines that may be used with p40-VL regions of the invention include other IGKV1D-16 loci germlines IGKV1D-16*02, IGKV1-16*01, IGKV1-16*02, IGKV1-39*01 (DPK9), IGKV1-12*01/1-12*02/1 D-12*01/1 D-12*02 (DPK5), IGKV1-9*01 (DPK8), IGKV1-5*03, IGKV1-5*01, IGKV1-27*01 (DPK4). The foregoing frameworks are modelled to be compatible with p40-VL CDRs of the invention.

In some aspects of the disclosure, the p40-CH1 of the antibody comprises a sequence selected from the group consisting of SEQ ID NO: 6, SEQ ID NO: 105, and SEQ ID NO: 110. In some aspects of the disclosure, the p40-CL of the antibody comprises a sequence selected from the group consisting SEQ ID NO: 16, SEQ ID NO: 108, and SEQ ID NO: 113. The p40-CH1 may comprise a sequence according to SEQ ID NO:16. The p40-CH1 may comprise a sequence according to SEQ ID NO: 6. The p40-CH1 and p40-CL may each be part of a multispecific antibody.

The p40-CH1 may be connected to the p40-VL, and the p40-CL may be connected to the p40-VH forming a p40-binding domain-swap domain (p40-xFab). Domain-swap Fabs are depicted in FIGS. 18 D, G, and H. Alternatively, the p40-CH1 may be connected to the p40-VH, and the p40-CL may be connected to the p40-VL forming an p40-binding Fab domain (p40-Fab). Fab domains are depicted in FIGS. 18 A, B, C, E, F, and I.

p40 antibodies of the invention may comprise a hinge region. The hinge region may be selected from any suitable sequence, including a sequence selected from any of Tables 82, 85, and 87. In some aspects, the hinge region is selected from the group consisting of SEQ ID NO: 7, SEQ ID NO: 102, SEQ ID NO: 113, SEQ ID NO:126, SEQ ID NO: 129, and SEQ ID NO:131.

The p40-CL may be connected to a hinge region which is then connected to a CH2 domain. Alternatively, the p40-CH1 may be connected to a hinge region which is then connected to a CH2 domain. The CH2 region may comprise a sequence selected from any one of Tables 86, and 87. The CH2 domain may comprise SEQ ID NO: 8. The CH2 region may be connected to a CH3 region. The CH3 region may comprise a sequence selected from any one of Tables 86, and 87. The CH3 region may comprise a sequence selected from the group consisting of SEQ ID NO: 9, SEQ ID NO: 106, SEQ ID NO: 111, SEQ ID NO: 114, SEQ ID NO: 117, SEQ ID NO: 124, SEQ ID NO: 127, SEQ ID NO: 139, SEQ ID NO: 141, SEQ ID NO: 147, and SEQ ID NO: 148.

The p40-VH bearing polypeptide may comprise a sequence selected from any one of Tables 86, and 87. The p40-VH bearing polypeptide may comprise a sequence at least 90%, 91%, 92%, 93%, 94%, 95%, 96%, 97%, 98%, 99%, or 100% identical to a sequence selected from the group consisting of SEQ ID NO: 170, SEQ ID NO: 177, SEQ ID NO: 181, SEQ ID NO: 185, and SEQ ID NO: 186.

The p40-VL bearing polypeptide may comprise a sequence selected from any one of Tables 86, and 87. The p40-VL bearing polypeptide may comprise a sequence at least 90%, 91%, 92%, 93%, 94%, 95%, 96%, 97%, 98%, 99%, or 100% identical to a sequence selected from the group consisting of SEQ ID NO: 176, SEQ ID NO: 178, SEQ ID NO: 179, SEQ ID NO: 182, and SEQ ID NO: 183.

In some aspects, the disclosure provides an isolated multimeric antibody that specifically binds p40 and at least one additional target selected from the group consisting of IL-4, IL-13, IL-33, and TSLP, comprising a heavy chain variable region (p40-VH) and a light chain variable region (p40-VL), wherein the p40-VH comprises the CDR-H1, CDR-H2, and CDR-H3 sequences of encoded by the plasmid deposited at the ATCC and having ATCC Accession No. PTA-127206, and the p40-VL comprises the CDR-L1, CDR-L2, and CDR-L3 sequences encoded by the plasmid deposited at the ATCC and having ATCC Accession No. PTA-127205.

In some aspects, the disclosure provides p40 antibodies comprising a p40-VH sequence encoded by the plasmid deposited at the ATCC and having ATCC Accession No. PTA-127206, and a p40-VL sequence encoded by the plasmid deposited at the ATCC and having ATCC Accession No. PTA-127205. In some aspects, the disclosure provides p40 antibodies comprising a p40-VH bearing polypeptide sequence encoded by the plasmid deposited at the ATCC and having ATCC Accession No. PTA-127204. In some aspects, the disclosure provides p40 antibodies comprising a p40-VL bearing polypeptide sequence encoded by the plasmid deposited at the ATCC and having ATCC Accession No. PTA-127203.

### Antibodies to IL-4

The disclosure provides antibodies that bind to IL-4. IL-4 antibodies may bind one or more additional targets. As used herein, the term “IL-4” includes variants, isoforms, homologs, orthologs and paralogs of one or more of IL-4. In some embodiments, an antibody disclosed herein cross-reacts with IL-4 from species other than human, such as IL-4 of cynomolgus monkey, as well as different forms of IL-4. In some embodiments, an antibody may be completely specific for human IL-4 and may not exhibit species cross-reactivity or other types of cross-reactivity. As used herein the term IL-4 refers to naturally occurring human IL-4 unless contextually dictated otherwise. An “IL-4 antibody” “anti-IL-4 antibody” or other similar designation means any antibody (as defined herein) that binds or reacts with IL-4, an isoform, fragment or derivative thereof. The full length, mature form of IL-4, is represented by UniProtKB/Swiss-Prot accession number P05112. The full length, mature form of murine IL-4, is represented by UniProtKB/Swiss-Prot accession number P07750. The full length, mature form of cynomologous IL-4, is represented by UniProtKB/Swiss-Prot accession number P79339.

In some embodiments, the invention provides an IL-4 antibody having a light chain variable region (VL) sequence and a heavy chain variable region (VH) sequence as each found in one or more of Table 80, 84, 85, 86, 87 or variants thereof.

The invention also provides CDR portions of antibodies to IL-4. Determination of CDR regions is defined in Example 1. In some embodiments, the antibody comprises three CDRs of any one of the IL-4 heavy chain variable regions shown in one or more of Table 80, 84, 85, 86, or 87. In some embodiments, the antibody comprises three CDRs of any one of the IL-4 light chain variable regions shown in one or more of Table 80, 84, 85, 86, or 87.

In some embodiments, the antibody comprises the six CDRs of an IL-4 antibody selected from one or more of Table 80, 84, 85, 86, and 87. In some embodiments, the antibody comprises the VH and VL of an IL-4 antibody each selected from one or more of Table 80, 84, 85, 86, and 87. In some embodiments, the antibody comprises the HC and LC of an IL-4 antibody each selected from one or more of Table 80, 84, 85, 86, and 87.

In some embodiments, the disclosure provides anti-IL-4 antibodies containing variations of the CDRs, VH, VL, HC, and LC regions shown in one or more of Table 80, 84, 85, 86, and 87, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 80, 84, 85, 86, and 87. These amounts are not meant to be limiting and increments between the recited percentages are specifically envisioned as part of the disclosure.

In some aspects, the disclosure provides an isolated antibody that specifically binds to IL-4, comprising a heavy chain variable region (IL4-VH) and a light chain variable region (IL4-VL), comprising
(i) a CDR-H1, CDR-H2, and CDR-H3 sequences of SEQ ID NO: 19, and a CDR-L1, CDR-L2, and CDR-L3 sequences of SEQ ID NO: 20;
(ii) a CDR-H1, CDR-H2, and CDR-H3 sequences of SEQ ID NO: 22, and a CDR-L1, CDR-L2, and CDR-L3 sequences of SEQ ID NO: 20;
(iii) a CDR-H1, CDR-H2, and CDR-H3 sequences of SEQ ID NO: 28, and a CDR-L1, CDR-L2, and CDR-L3 sequences of SEQ ID NO: 29;
(iv) a CDR-H1, CDR-H2, and CDR-H3 sequences of SEQ ID NO: 22, and a CDR-L1, CDR-L2, and CDR-L3 sequences of SEQ ID NO: 30; or
(v) a CDR-H1, CDR-H2, and CDR-H3 sequences of SEQ ID NO: 22, and a CDR-L1, CDR-L2, and CDR-L3 sequences of SEQ ID NO: 26.

In some aspects, the disclosure provides an isolated antibody that specifically binds to IL-4, comprising a heavy chain variable region (IL4-VH) and a light chain variable region (IL4-VL), comprising the CDR-H1, CDR-H2, and CDR-H3 sequences of SEQ ID NO: 22, and the CDR-L1, CDR-L2, and CDR-L3 sequences of SEQ ID NO: 26.

In some aspects, the disclosure provides an isolated antibody that specifically binds to IL-4, comprising a heavy chain variable region (IL4-VH) and a light chain variable region (IL4-VL), wherein the CDR-H1 comprises the amino acid sequence of SEQ ID NO: 18; the CDR-H2 comprises the amino acid sequence of SEQ ID NO: 2; the CDR-H3 comprises the amino acid sequence of SEQ ID NO: 3; the CDR-L1 comprises the amino acid sequence of SEQ ID NO: 24; the CDR-L2 comprises the amino acid sequence of SEQ ID NO: 12, and the CDR-L3 comprises the amino acid sequence of SEQ ID NO: 25.

The IL-4 antibody may comprise an IL4-VH framework sequence comprising a human germline VH framework sequence. The IL4-VH framework sequence may comprise one or more amino acid substitutions, additions, or deletions, while still retaining functional and structural similarity with the germline from which it was derived. In some aspects, the VH framework is at least 90%, at least 91%, at least 92%, at least 93%, at least 94%, at least 95%, at least 96%, at least 97%, at least 98%, at least 99%, or 100% identical to a human germline VH framework sequence. In some aspects, the IL-4 antibody comprises an IL4-VH framework sequence comprising 1, 2, 3, 4, 5, 6, 7, 8, 9, 10 amino acid substitutions, additions or deletions relative to the human germline VH framework sequence. In some aspects, the 1, 2, 3, 4, 5, 6, 7, 8, 9, or 10 amino acid substitutions, additions or deletions are only in the framework regions. In some aspects, the % identity is based on similarity with VH excluding those portions herein defined as CDRs.

In some aspects, the IL4-VH framework sequence may be derived from a human germline VH sequence selected from the group consisting of DP26, DP27, DP28, and DP76. In some aspects, the IL4-VH framework sequence is derived from DP76.

The IL4-VH framework sequence may be derived from a human germline VH sequence selected from the group consisting of IGHV2-5*02, IGHV2-5*08, IGHV2-5*09, IGHV2-5*05/2-5*06, IGHV2-5*01, IGHV2-70D*04/2-70D*14, IGHV2-70*11, IGHV2-70*01/2-70*13, IGHV2-70*10, IGHV2-70*12, and IGHV2-26*01. In some aspects, the IL4-VH framework sequence is derived from IGHV2-5*02.

The invention has identified the human germline VH framework IGHV2-5*02 (DP-76) as highly advantageous with IL4-VH CDRs of the invention. Other germlines are modelled to be advantageous with the IL4-VH CDRs of the invention, including IGHV2-5 loci germlines such as IGHV2-5*08, IGHV2-5*09, IGHV2-5*05/2-5*06 and IGHV2-5*01; IGHV2-70D*04/2-70D*14 (DP-28) and other IGHV2-7 loci germlines including IGHV2-70*11, IGHV2-70*01/2-70*13 (DP-27), IGHV2-70*10, IGHV2-70*12, and IGHV2-26*01 (DP-26). The foregoing frameworks are modelled to be compatible with IL4-VH CDRs of the invention.

The IL-4 antibody may comprise an IL4-VL framework sequence comprising a human germline VL framework sequence. The IL4-VL framework sequence may comprise one or more amino acid substitutions, additions, or deletions, while still retaining functional and structural similarity with the germline from which it was derived. In some aspects, the VL framework is at least 90%, at least 91%, at least 92%, at least 93%, at least 94%, at least 95%, at least 96%, at least 97%, at least 98%, at least 99%, or 100% identical to a human germline VL framework sequence. In some aspects, the IL-4 antibody comprises an IL4-VL framework sequence comprising 1, 2, 3, 4, 5, 6, 7, 8, 9, 10 amino acid substitutions, additions or deletions relative to the human germline VL framework sequence. In some aspects, the 1, 2, 3, 4, 5, 6, 7, 8, 9, or 10 amino acid substitutions, additions or deletions are only in the framework regions. In some aspects, the % identity is based on similarity with VL excluding those portions herein defined as CDRs.

In some aspects, the IL4-VL framework sequence may be derived from a human germline VL sequence selected from the group consisting of DPK1, DPK3, DPK4, DPK5, DPK7, DPK8, DPK9, and DPK24. In some aspects, the IL4-VL framework sequence is derived from DPK9.

The IL4-VL framework sequence may be derived from a human germline VL sequence selected from the group consisting of IGKV1-39*01, IGKV4-1*01, 1D-39*01, IGKV1-12*01, IGKV1-9*01, IGKV1-16*01, IGKV1-16*02, IGKV1-33*01/1D-33*01, IGKV1-27*01, IGKV1D-16*01, IGKV1-13*02/1D-13*02, IGKV1-17*01, IGKV1-17*02, IGKV1-17*03, and IGKV1-6*01/1-6*02. In some aspects, the IL4-VL framework sequence is derived from IGKV1-39*01.

The invention has identified the human germline VL framework DPK9 (IGKV1-39*01) as highly advantageous with IL4-VL CDRs of the invention. IGKV4-1*01 (DPK24) is also predicted to be highly advantageous, as this functions well with GSK 3B9 VL. Other germlines are modelled to be advantageous with the IL4-VL CDRs of the invention, including the group consisting of 1D-39*01, IGKV1-12*01 (DPK5), IGKV1-9*01 (DPK8), IGKV1-16*01, IGKV1-16*02, IGKV1-33*01/1D-33*01 (DPK1), IGKV1-27*01 (DPK4), IGKV1D-16*01 (DPK7), IGKV1-13*02/1D-13*02, IGKV1-17*01, IGKV1-17*02, IGKV1-17*03, and IGKV1-6*01/1-6*02 (DPK3). The foregoing frameworks are modelled to be compatible with IL4-VL CDRs of the invention.

In some aspects of the disclosure, the IL4-CH1 of the antibody comprises a sequence selected from the group consisting of SEQ ID NO: 6, SEQ ID NO: 105, and SEQ ID NO: 110. In some aspects of the disclosure, the IL4-CL of the antibody comprises a sequence selected from the group consisting SEQ ID NO: 16, SEQ ID NO: 108, and SEQ ID NO: 113. The IL4-CH1 may comprise a sequence according to SEQ ID NO:16. The IL4-CH1 may comprise a sequence according to SEQ ID NO: 6. The IL4-CH1 and IL4-CL may each be part of a multispecific antibody.

The IL4-CH1 may be connected to the IL4-VL, and the IL4-CL may be connected to the IL4-VH forming an IL-4-binding domain-swap Fab domain (IL4-xFab). Domain-swap Fabs are depicted in FIGS. 18 D, G, and H. Alternatively, the IL4-CH1 may be connected to the IL4-VH, and the IL4-CL may be connected to the IL4-VL forming an IL-4-binding Fab domain (IL4-Fab). Fab domains are depicted in FIGS. 18 A, B, C, E, F, and I.

IL-4 antibodies of the invention may comprise a hinge region. The hinge region may be selected from any suitable sequence, including a sequence selected from any of Tables 80, 84, 85, 86, and 87. In some aspects, the hinge region is selected from the group consisting of SEQ ID NO: 7, SEQ ID NO: 102, SEQ ID NO: 123, SEQ ID NO:126, SEQ ID NO: 129, and SEQ ID NO:131.

The IL4-CL may be connected to a hinge region which is then connected to a CH2 domain. Alternatively, the IL4-CH1 may be connected to a hinge region which is then connected to a CH2 domain. The CH2 region may comprise a sequence selected from any one of Tables 80, 84, 85, 86, and 87. The CH2 domain may comprise SEQ ID NO: 8. The CH2 region may be connected to a CH3 region. The CH3 region may comprise a sequence selected from any one of Tables 80, 84, 85, 86, and 87. The CH3 region may comprise a sequence selected from the group consisting of SEQ ID NO: 9, SEQ ID NO: 106, SEQ ID NO: 111, SEQ ID NO: 114, SEQ ID NO: 117, SEQ ID NO: 124, SEQ ID NO: 127, SEQ ID NO: 139, SEQ ID NO: 141, SEQ ID NO: 147, and SEQ ID NO: 148.

The IL4-VH bearing polypeptide may comprise a sequence selected from any one of Tables 80, 84, 85, 86, and 87. The IL4-bearing polypeptide may comprise a sequence at least 90%, 91%, 92%, 93%, 94%, 95%, 96%, 97%, 98%, 99%, or 100% identical to a sequence selected from the group consisting of SEQ ID NO: 23, SEQ ID NO: 107, SEQ ID NO: 115, SEQ ID NO: 121, SEQ ID NO: 125, SEQ ID NO: 130, SEQ ID NO: 133, SEQ ID NO: 135, SEQ ID NO: 140, SEQ ID NO: 144, SEQ ID NO: 146, SEQ ID NO: 151, SEQ ID NO: 153, SEQ ID NO: 153, SEQ ID NO: 156, SEQ ID NO: 157, SEQ ID NO: 159, SEQ ID NO: 162, and SEQ ID NO: 164, SEQ ID NO: 179, SEQ ID NO: 180, and SEQ ID NO: 183.

The IL4-VL bearing polypeptide may comprise a sequence selected from any one of Tables 90, 84, 85, 86, and 87. The IL4-VL bearing polypeptide may comprise a sequence at least 90%, 91%, 92%, 93%, 94%, 95%, 96%, 97%, 98%, 99%, or 100% identical to a sequence selected from the group consisting of SEQ ID NO: 27, SEQ ID NO: 109, and SEQ ID NO: 116, SEQ ID NO: 136, SEQ ID NO: 197, and SEQ ID NO: 208.

IL-4 antibodies of the disclosure advantageously bind to both human and cynomolgus monkey within one order of magnitude or less. This facilitates using animal and toxicology data to provide inform human dosing. The IL-4 antibodies of the invention advantageously have improved binding affinity and IL-4 neutralization compared to the parental antibody.

The IL-4 antibodies of the present disclosure demonstrate reduced post-translational isomerization at CDRL1 residues 28 and 29 compared to the parental antibody. In some aspects, the reduced post translational isomerization is detected by incubation in Glutamic acid at pH 4.5 and Tris at pH 7.5, and then the samples subjected to double digestion by LysC and Trypsin and analyzed on LC/MS using a high-fidelity method with a Lumos C18 column.

The IL-4 antibodies of the present disclosure demonstrate reduced viscosity over parental antibodies, as measured by the Anton Paar method. In some aspects, the Anton Paar method uses a CP25-1 cone and plate on a MCR-302 rheometer at a constant rotational speed of 150 rpm at 25° C.

In some aspects, the disclosure provides an isolated antibody that specifically binds IL-4 comprising a heavy chain variable region (IL4-VH) and a light chain variable region (IL4-VL), wherein the IL4-VH comprises the CDR-H1, CDR-H2, and CDR-H3 sequences of encoded by the plasmid deposited at the ATCC and having ATCC Accession No. PTA-127198, and the IL4-VL comprises the CDR-L1, CDR-L2, and CDR-L3 sequences encoded by the plasmid deposited at the ATCC and having ATCC Accession No. PTA-127197.

In some aspects, the disclosure provides anti-IL-4 antibodies comprising the IL4-VH sequence encoded by the plasmid deposited at the ATCC and having ATCC Accession No. PTA-127198. In some aspects, the disclosure provides anti-IL-4 antibodies comprising the IL4-VL sequence encoded by the plasmid deposited at the ATCC and having ATCC Accession No. PTA-127197. In some aspects, the disclosure provides anti-IL-4 antibodies comprising the IL4-VH bearing polypeptide sequence encoded by the plasmid deposited at the ATCC and having ATCC Accession No. PTA-127192. In some aspects, the disclosure provides anti-IL-4 antibodies comprising the IL4-VL bearing polypeptide sequence encoded by the plasmid deposited at the ATCC and having ATCC Accession No. PTA-127194.

### Antibodies to IL-13

The disclosure provides antibodies that bind to IL-13. IL-13 antibodies may bind one or more additional targets. As used herein, the term “IL-13” includes variants, isoforms, homologs, orthologs and paralogs of one or more of IL-13. In some embodiments, an antibody disclosed herein cross-reacts with IL-13 from species other than human, such as IL-13 of cynomolgus monkey, as well as different forms of IL-13. In some embodiments, an antibody may be completely specific for human IL-13 and may not exhibit species cross-reactivity or other types of cross-reactivity. As used herein the term IL-13 refers to naturally occurring human IL-13 unless contextually dictated otherwise. An “IL-13 antibody” “anti-IL-13 antibody” or other similar designation means any antibody (as defined herein) that binds or reacts with IL-13, an isoform, fragment or derivative thereof. The full length, mature form of IL-13, is represented by UniProtKB/Swiss-Prot accession number P35225. The full length, mature form of murine IL-13, is represented by UniProtKB/Swiss-Prot accession number P20109. The full length, mature form of cynomologous IL-13, is represented by UniProtKB/Swiss-Prot accession number Q0PW92.

In some embodiments, the invention provides an IL-13 antibody having a light chain variable region (VL) sequence and a heavy chain variable region (VH) sequence as each found in one or more of Table 81, 84, 85, 86, and 87, or variants thereof.

The invention also provides CDR portions of antibodies to IL-13. Determination of CDR regions is defined in Example 1. In some embodiments, the antibody comprises three CDRs of any one of the heavy chain variable regions shown in one or more of Table 81, 84, 85, 86, and 87. In some embodiments, the antibody comprises three CDRs of any one of the light chain variable regions shown one or more of Table 81, 84, 85, 86, and 87. In some embodiments, the antibody comprises three CDRs of any one of the heavy chain variable regions and three CDRs of any one of the light chain variable regions each shown in one or more of Table 81, 84, 85, 86, and 87.

In some embodiments, the antibody comprises the six CDRs of an IL-13 antibody selected from one or more of Table 81, 84, 85, 86, and 87. In some embodiments, the antibody comprises the VH and VL of an IL-13 antibody each selected from one or more of Table 81, 84, 85, 86, and 87. In some embodiments, the antibody comprises the HC and LC of an IL-13 antibody each selected from one or more of Table 81, 84, 85, 86, and 87.

In some embodiments, the disclosure provides anti-IL-13 antibodies containing variations of the CDRs, VH, VL, HC, and LC regions shown in one or more of Table 81, 84, 85, 86, and 87, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 81, 84, 85, 86, and 87. These amounts are not meant to be limiting and increments between the recited percentages are specifically envisioned as part of the disclosure.

In some aspects, the disclosure provides an isolated antibody that specifically binds to IL-13, comprising a heavy chain variable region (IL13-VH) and a light chain variable region (IL13-VL), comprising
(i) the CDR-H1, CDR-H2, and CDR-H3 sequences of SEQ ID NO: 44, and the CDR-11, CDR-L2, and CDR-L3 sequences of SEQ ID NO: 46;
(ii) the CDR-H1, CDR-H2, and CDR-H3 sequences of SEQ ID NO: 48, and the CDR-11, CDR-L2, and CDR-L3 sequences of SEQ ID NO: 49;
(iii) the CDR-H1, CDR-H2, and CDR-H3 sequences of SEQ ID NO: 48, and the CDR-11, CDR-L2, and CDR-L3 sequences of SEQ ID NO: 68;
(iv) the CDR-H1, CDR-H2, and CDR-H3 sequences of SEQ ID NO: 57, and the CDR-11, CDR-L2, and CDR-L3 sequences of SEQ ID NO: 59; or
(v) the CDR-H1, CDR-H2, and CDR-H3 sequences of SEQ ID NO: 51, and the CDR-11, CDR-L2, and CDR-L3 sequences of SEQ ID NO: 54.

In some aspects, the disclosure provides an isolated antibody that specifically binds to IL-13, comprising a heavy chain variable region (IL13-VH) and a light chain variable region (IL13-VL), comprising the CDR-H1, CDR-H2, and CDR-H3 sequences of SEQ ID NO: 51, and the CDR-L1, CDR-L2, and CDR-L3 sequences of SEQ ID NO: 54.

In some aspects, the disclosure provides an isolated antibody that specifically binds to IL-13, comprising a heavy chain variable region (IL13-VH) and a light chain variable region (IL13-VL), wherein the CDR-H1 comprises the amino acid sequence of SEQ ID NO: 41; the CDR-H2 comprises the amino acid sequence of SEQ ID NO: 42; the CDR-H3 comprises the amino acid sequence of SEQ ID NO:-50; the CDR-L1 comprises the amino acid sequence of SEQ ID NO: 53; the CDR-L2 comprises the amino acid sequence of SEQ ID NO: 37, and the CDR-L3 comprises the amino acid sequence of SEQ ID NO: 38.

The IL-13 antibody may comprise an IL13-VH framework sequence comprising a human germline VH framework sequence. The IL13-VH framework sequence may comprise one or more amino acid substitutions, additions, or deletions, while still retaining functional and structural similarity with the germline from which it was derived. In some aspects, the VH framework is at least 90%, at least 91%, at least 92%, at least 93%, at least 94%, at least 95%, at least 96%, at least 97%, at least 98%, at least 99%, or 100% identical to a human germline VH framework sequence. In some aspects, the IL-13 antibody comprises an IL13-VH framework sequence comprising 1, 2, 3, 4, 5, 6, 7, 8, 9, 10 amino acid substitutions, additions or deletions relative to the human germline VH framework sequence. In some aspects, the 1, 2, 3, 4, 5, 6, 7, 8, 9, or 10 amino acid substitutions, additions or deletions are only in the framework regions. In some aspects, the % identity is based on similarity with VH excluding those portions herein defined as CDRs.

In some aspects, the IL13-VH framework sequence may be derived from a human germline VH sequence selected from the group consisting of DP7, DP10, DP35, DP47, DP50, DP51, DP54, and DP77. In some aspects, the IL13-VH framework sequence is derived from DP54.

The IL13-VH framework sequence may be derived from a human germline VH sequence selected from the group consisting of IGHV3-7*01, IGHV3-7*02, IGHV3-7*03, IGHV3-23*01, IGHV3-23*03, IGHV3-48*01, IGHV3-48*02, IGHV3-21*01, IGHV3-11*01, IGHV3-53*01, IGHV3-64*04, IGHV3-33*01, IGHV1-46*01/1-46*03, and IGHV1-69*01/1-69D*01/1-69*12/1-69*13.

The invention has identified the human germline VH framework IGHV3-7*01 (DP54) as particularly advantageous with IL13-VH CDRs of the invention. Other advantageous germlines for grafting IL-13V_{H} CDRs include germlines from IGHV3-7 loci (e.g., IGHV3-7*02 and IGHV3-7*03), IGHV3-23*01 (DP-47) and other germlines from IGHV3-23 loci including IGHV3-23*03, IGHV3-48*01 and other germlines from IGHV3-48 loci including IGHV3-48*02 (DP-51), IGHV3-21*01 (DP-77), IGHV3-11*01 (DP-35) and other germlines from IGHV3-11 loci, IGHV3-53*01, IGHV3-64*04 and other germlines from IGHV3-64 loci, IGHV3-33*01 (DP-50) and other germlines from the IGHV3-33 loci, IGHV1-46*01/1-46*03 (DP-7) and other germlines from IGHV1-46 loci, IGHV1-69*01/1-69D*01/1-69*12/1-69*13 (DP-10) and other germlines from IGHV1-69 loci (DP-76). The foregoing frameworks are modelled to be compatible with IL13-VH CDRs of the invention.

The IL-13 antibody may comprise an IL13-VL framework sequence comprising a human germline VL framework sequence. The IL13-VL framework sequence may comprise one or more amino acid substitutions, additions, or deletions, while still retaining functional and structural similarity with the germline from which it was derived. In some aspects, the VL framework is at least 90%, at least 91%, at least 92%, at least 93%, at least 94%, at least 95%, at least 96%, at least 97%, at least 98%, at least 99%, or 100% identical to a human germline VL framework sequence. In some aspects, the IL-13 antibody comprises an IL13-VL framework sequence comprising 1, 2, 3, 4, 5, 6, 7, 8, 9, 10 amino acid substitutions, additions or deletions relative to the human germline VL framework sequence. In some aspects, the 1, 2, 3, 4, 5, 6, 7, 8, 9, or 10 amino acid substitutions, additions or deletions are only in the framework regions. In some aspects, the % identity is based on similarity with VL excluding those portions herein defined as CDRs.

In some aspects, the IL13-VL framework sequence may be derived from a human germline VL sequence selected from the group consisting of DPK3, DPK4, DPK5, DPK8, DPK9, DPK10, DPK23. In some aspects, the IL13-VL framework sequence is derived from DPK9.

The IL13-VL framework sequence may be derived from a human germline VL sequence selected from the group consisting of IGKV1-39*01, 1D-39*01, IGKV1-12*01, IGKV1-9*01, IGKV1-5*01, IGKV1-27*01, IGKV1D-16*02, IGKV1-17*01, IGKV1-17*02, IGKV1-17*03, IGKV1-6*01/1-6*02, IGKV1D-8*01/1D-8*03, and IGKV3D-7*01. In some aspects, the IL13-VL framework sequence is derived from IGHV3-7*01.

The invention has identified the human germline VL framework DPK9 (IGKV1-39*01) as highly advantageous with IL13-VL CDRs of the invention. Advantageously, other VL germlines that may be used with IL13-VL regions of the invention include 1D-39*01, IGKV1-12*01 (DPK5) and other germlines from the IGKV1-12 loci, IGKV1-9*01 (DPK8), IGKV1-5*01 and other IGKV1-5 loci germlines; IGKV1-27*01 (DPK4), IGKV1D-16*02, IGKV1-17*01, IGKV1-17*02, IGKV1-17*03, IGKV1-6*01/1-6*02 (DPK3), IGKV1D-8*01/1D-8*03 (DPK10) and IGKV3D-7*01 (DPK23). The foregoing frameworks are modelled to be compatible with IL13-VL CDRs of the invention.

In some aspects of the disclosure, the IL13-CH1 of the antibody comprises a sequence selected from the group consisting of SEQ ID NO: 6, SEQ ID NO: 105, and SEQ ID NO: 110. In some aspects of the disclosure, the IL13-CL of the antibody comprises a sequence selected from the group consisting SEQ ID NO: 16, SEQ ID NO: 108, and SEQ ID NO: 113. The IL13-CH1 may comprise a sequence according to SEQ ID NO:16. The IL13-CH1 may comprise a sequence according to SEQ ID NO: 6. The IL13-CH1 and IL13-CL may each be part of a multispecific antibody.

The IL13-CH1 may be connected to the IL13-VL, and the IL13-CL may be connected to the IL13-VH forming an IL-13-binding domain-swap Fab domain (IL13-xFab). Domain-swap Fabs are depicted in FIGS. 18 D, G, and H. Alternatively, the IL13-CH1 may be connected to the IL13-VH, and the IL13-CL may be connected to the IL31-VL forming an IL-33-binding Fab domain (IL13-Fab). Fab domains are depicted in FIGS. 18 A, B, C, E, F, and I. IL-13 antibodies of the invention may comprise a hinge region. The hinge region may be selected from any suitable sequence, including a sequence selected from any of Tables 82, 85, and 87. In some aspects, the hinge region is selected from the group consisting of SEQ ID NO: 7, SEQ ID NO: 102, SEQ ID NO: 123, SEQ ID NO:126, SEQ ID NO: 129, and SEQ ID NO:131.

The IL13-CL may be connected to a hinge region which is then connected to a CH2 domain. Alternatively, the IL13-CH1 may be connected to a hinge region which is then connected to a CH2 domain. The CH2 region may comprise a sequence selected from any one of Tables 81, 84, 85, 86, and 87. The CH2 domain may comprise SEQ ID NO: 8. The CH2 region may be connected to a CH3 region. The CH3 region may comprise a sequence selected from any one of Tables 81, 84, 85, 86, and 87. The CH3 region may comprise a sequence selected from the group consisting of SEQ ID NO: 9, SEQ ID NO: 106, SEQ ID NO: 111, SEQ ID NO: 114, SEQ ID NO: 117, SEQ ID NO: 124, SEQ ID NO: 127, SEQ ID NO: 139, SEQ ID NO: 141, SEQ ID NO: 147, and SEQ ID NO: 148.

The IL13-VH bearing polypeptide may comprise a sequence selected from any one of Tables 81, 84, 85, 86, and 87. The IL13-VH bearing polypeptide may comprise a sequence at least 90%, 91%, 92%, 93%, 94%, 95%, 96%, 97%, 98%, 99%, or 100% identical to a sequence selected from the group consisting of SEQ ID NO: 52, SEQ ID NO:66, SEQ ID NO: 112, SEQ ID NO: 118, SEQ ID NO: 121, SEQ ID NO: 122, SEQ ID NO: 130, SEQ ID NO: 145, SEQ ID NO: 149, SEQ ID NO: 152, SEQ ID NO: 154, SEQ ID NO:160, SEQ ID NO: 162, and SEQ ID NO: 164, and SEQ ID NO: 209.

The IL13-VL bearing polypeptide may comprise a sequence selected from any one of Tables 81, 84, 85, 86, and 87. The IL13-VL bearing polypeptide may comprise a sequence at least 90%, 91%, 92%, 93%, 94%, 95%, 96%, 97%, 98%, 99%, or 100% identical to a sequence selected from the group consisting of SEQ ID NO: 55, SEQ ID NO: 119, SEQ ID NO: 120, SEQ ID NO: 125, SEQ ID NO: 130, SEQ ID NO: 133, SEQ ID NO: 135, SEQ ID NO: 140, SEQ ID NO: 163, SEQ ID NO: 164, and SEQ ID NO: 196.

IL-13 antibodies of the disclosure advantageously bind to both human and cynomolgus monkey within one order of magnitude or less. Antibodies with favorable binding ratios between human and their cynomolgus counterpart facilitate using animal and toxicology data to inform human dosing. The IL-13 antibodies of the invention advantageously have improved binding affinity and IL-13 neutralization compared to known antibodies.

The IL-13 antibodies of the present disclosure demonstrate reduced non-germline T-cell epitopes compared to known IL-13 antibodies. Advantageously, the IL-13 antibodies of the disclosure combine reduced T-cell epitopes with retention of K within an order of magnitude. In some aspects, the disclosure provides an isolated antibody that specifically binds IL-13 comprising a heavy chain variable region (IL13-VH) and a light chain variable region (IL13-VL), wherein the IL13-VH comprises the CDR-H1, CDR-H2, and CDR-H3 sequences of encoded by the plasmid deposited at the ATCC and having ATCC Accession No. PTA-127196, and the IL13-VL comprises the CDR-L1, CDR-L2, and CDR-L3 sequences encoded by the plasmid deposited at the ATCC and having ATCC Accession No. PTA-127195.

In some aspects, the disclosure provides anti-IL-13 antibodies comprising the IL13-VH sequence encoded by the plasmid deposited at the ATCC and having ATCC Accession No. PTA-127196. In some aspects, the disclosure provides anti-IL-13 antibodies comprising the IL13-VL sequence encoded by the plasmid deposited at the ATCC and having ATCC Accession No. PTA-127195. In some aspects, the disclosure provides anti-IL-13 antibodies comprising the IL13-VH bearing polypeptide sequence encoded by the plasmid deposited eat the ATCC and having ATCC Accession No. PTA-127193. In some aspects, the disclosure provides anti-IL-13 antibodies comprising the IL13-VL bearing polypeptide sequence encoded by the plasmid deposited at the ATCC and having ATCC Accession No. PTA-127192.

### Antibodies to IL-4/IL-13/IL-33

IL-33 is a cytokine alarmin stored in the nuclei of epithelial cells, keratinocytes, endothelial cells, and fibroblasts, and is released upon cellular damage to mediate type 2 inflammatory responses (66, 67). IL-33 binds to a heterodimeric receptor comprised of ST2 (IL1RL1) and IL-1RAcP on T cells, ILC2, basophils, mast cells, eosinophils, and other cell types, to engage NFkB and MAPK pathways through MyD88, and drive production of IL-13, IL-5, and other downstream cytokines. Soluble ST2 (sST2) found in circulation acts as a decoy receptor for IL-33 (68). IL-4 and IL-13 can activate IL-33 producing cells, while IL-33 responses lead to generation of type 2 cytokines, thus establishing an inflammatory cascade (69).

Anti-IL-33 mAb Itepekimab (REGN3500; Sanofi/Regeneron) effectively reduced AD extent and severity above placebo (NCT03738423) but appeared less potent than Dupixent® (NCT03736967). Several other IL-33-targeted agents (anti-IL-33 mAb Etokimab, anti-ST2 CNTO-7160) lacked significant clinical efficacy in AD (70, 71). In AD, the combination of itepekimab and Dupixent® was not superior to Dupixent® alone in reducing disease severity, but there was a trend toward greater reduction of pruritus with the combination than with either treatment alone (NCT03736967). In asthma, Itepekimab significantly improved asthma control and lung function, but was not superior to Dupixent® (72). In COPD, Itepekimab reduced exacerbation rate and improved lung function in former smokers (73).

At the barrier surfaces, epithelial damage releases alarmins TSLP and IL-33, which promote a cascade of downstream effector responses, including local generation of IL-4, IL-5, and IL-13. In turn, these cytokines promote and sustain epithelial dysfunction, resulting in an escalating cycle of tissue damage, inflammation, fibrosis, and itch (41, 69, 74).

IL13433-1258 and other IL-4/IL-13/TSLP antibodies were designed to achieve combinatorial blockade of three distinct, clinically validated pathways for treatment of atopic disorders. IL13433-1258 in particular neutralizes IL-4 and IL-13 with approximately 10-fold greater potency than Dupixent®. IL13433-1258 includes an IL-33 binding domain with activity comparable to Itepekimab and has the potential to offer more complete inhibition of type 2 effector responses, including itch, compared to IL-4/13 neutralization alone.

The disclosure provides antibodies that bind to IL-4, IL-13, and IL-33. As used herein, the terms IL-4, IL-13, and IL-33 include variants, isoforms, homologs, orthologs and paralogs of one or more of IL-4, IL-13, and IL-33 respectively. In some embodiments, an antibody disclosed herein cross-reacts with one or more of IL-4, IL-13, and IL-33 from species other than human, such as IL-4, IL-13, and IL-33 of cynomolgus monkey. In some embodiments, an antibody may be completely specific for human IL-4, IL-13, and IL-33 and may not exhibit species cross-reactivity or other types of cross-reactivity. As used herein the term IL-4, IL-13, and IL-33 refers to naturally occurring human IL-4, IL-13, and IL-33 unless contextually dictated otherwise. An “IL-4/IL-13/IL-33 antibody” “anti-IL-4/IL-13/IL-33 antibody” or other similar designation means any antibody (as defined herein) that binds or reacts with IL-4, IL-13, and IL-33, an isoform, fragment or derivative thereof.

In some embodiments, the invention provides an IL-4/IL-13/IL-33 antibody having a light chain variable region (VL) sequence and a heavy chain variable region (VH) sequence as found in Table 85, or variants thereof.

The invention also provides CDR portions of IL-4/IL-13/IL-33 antibodies. Determination of CDR regions is defined in Example 1. In some embodiments, the IL-4/IL-13/IL-33 antibody comprises the six CDRs of an IL-4 antibody selected from one of Table 80 and 85, the six CDRs of an IL-13 antibody selected from one of Table 81 and 85, and the six CDRs of an IL-33 antibody selected from one of Table 82 and 85. In some embodiments, the antibody comprises the VH and VL of an IL-4 antibody selected from Table 80 and 85, the VH and VL of an IL-13 antibody selected from Table 81 or Table 85, and the VH and VL of an IL-33 antibody selected from Table 82 or Table 85. In some embodiments, the IL-4/IL-13/IL-33 antibody comprises sequences selected from Table 85.

In some embodiments, the disclosure provides anti-IL-4/IL-13/IL-33 antibodies containing variations of the CDRs, VH, VL, HC, and LC regions shown in one or more of Table 80, 81, 82, 85, and 87, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 80, 81, 82, 85, and 87. These amounts are not meant to be limiting and increments between the recited percentages are specifically envisioned as part of the disclosure.

The present disclosure relates to an isolated antibody that specifically binds to IL-33, specifically binds to IL-4, and specifically binds to IL-13, comprising an IL-33 binding domain, an IL-4 binding domain, and an IL-13 binding domain. The IL-4/IL-13/IL-33 antibody may comprise
(i) an IL-33 binding domain comprising a heavy chain variable region (IL33-VH) and a light chain variable region (IL33-VL), wherein the CDR-H1 of the IL-33 binding domain comprises the amino acid sequence of SEQ ID NO: 60; the CDR-H2 of the IL-33 binding domain comprises the amino acid sequence of SEQ ID NO: 61; the CDR-H3 of the IL-33 binding domain comprises the amino acid sequence of SEQ ID NO: 72; the CDR-L1 of the IL-33 binding domain comprises the amino acid sequence of SEQ ID NO: 75; the CDR-L2 of the IL-33 binding domain comprises the amino acid sequence of SEQ ID NO: 76, and the CDR-L3 of the IL-33 binding domain comprises the amino acid sequence of SEQ ID NO: 77; and
(ii) an IL-4 binding domain comprising a heavy chain variable region (IL4-VH) and a light chain variable region (IL4-VL), wherein the CDR-H1 of the IL-4 binding domain comprises the amino acid sequence of SEQ ID NO: 18; the CDR-H2 of the IL-4 binding domain comprises the amino acid sequence of SEQ ID NO: 2; the CDR-H3 of the IL-4 binding domain comprises the amino acid sequence of SEQ ID NO: 3; the CDR-L1 of the IL-4 binding domain comprises the amino acid sequence of SEQ ID NO: 24; the CDR-L2 of the IL-4 binding domain comprises the amino acid sequence of SEQ ID NO: 12, and the CDR-L3 of the IL-4 binding domain comprises the amino acid sequence of SEQ ID NO: 25, and
(iii) an IL-13 binding domain comprising a heavy chain variable region (IL13-VH) and a light chain variable region (IL13-VL), wherein the CDR-H1 of the IL-13 binding domain comprises the amino acid sequence of SEQ ID NO: 41; the CDR-H2 of the IL-13 binding domain comprises the amino acid sequence of SEQ ID NO: 42; the CDR-H3 of the IL-13 binding domain comprises the amino acid sequence of SEQ ID NO:-50; the CDR-L1 of the IL-13 binding domain comprises the amino acid sequence of SEQ ID NO: 53; the CDR-L2 of the IL-13 binding domain comprises the amino acid sequence of SEQ ID NO: 37, and the CDR-L3 of the IL-13 binding domain comprises the amino acid sequence of SEQ ID NO: 38.

In some aspects of the disclosure, the IL-4/IL-13/IL-33 antibody may comprise
(i) an IL-33 binding domain comprising an IL33-VH of SEQ ID NO: 73, and an IL33-VL of SEQ ID NO: 78;
(ii) an IL-4 binding domain comprising an IL4-VH of SEQ ID NO: 22, and an IL4-VL of SEQ ID NO: 26;
(iii) an IL-13 binding domain comprising an IL13-VH of SEQ ID NO: 51, and an IL13-VL of SEQ ID NO: 54.

The IL-33 binding domain may be fused to the IL-4 binding domain. The IL-33 binding domain may be fused to the IL-13 binding domain. The IL-13 binding domain may be fused to the IL-4 binding domain. The fusion may be with or without a linker. If present, the linker may comprise a sequence of amino acids between 2 and 20 amino acids in length. The linker may consist of one or more amino acids selected from glycine, alanine, and serine. The linker may consist of one or more amino acids selected from glycine and serine. The linker may comprise SEQ ID NO: 104.

IL-4/IL-13/IL-33 antibodies of the invention may comprise a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site.

In some aspects, the first antigen binding site specifically binds IL-4, the second antibody binding site specifically binds IL-13, and the third antigen binding site specifically binds IL-33. In some aspects, the first antigen binding site specifically binds IL-4, the second antibody binding site specifically binds IL-33, and the third antigen binding site specifically binds IL-13. In some aspects, the first antigen binding site specifically binds IL-13, the second antibody binding site specifically binds IL-4, and the third antigen binding site specifically binds IL-33. In some aspects, the first antigen binding site specifically binds IL-13, the second antibody binding site specifically binds IL-33, and the third antigen binding site specifically binds IL-4. In some aspects, the first antigen binding site specifically binds IL-33, the second antibody binding site specifically binds IL-13, and the third antigen binding site specifically binds IL-4. In some aspects, the first antigen binding site specifically binds IL-33, the second antibody binding site specifically binds IL-4, and the third antigen binding site specifically binds IL-13.

The IL-4/IL-13/IL-33 antibody may comprise one or more domain-swap Fab domains. The domain-swap Fab domains may be selected from the group consisting of IL33-xFab, IL4-xFab, and IL13-xFab.

The IL-4/IL-13/IL-33 antibody may comprise a first Fab domain comprising the IL13-Fab, a second Fab domain comprising IL4-Fab, and a third Fab domain comprising the IL33-Fab.

To facilitate formation of the multispecific antibody, the first polypeptide may comprises a first Fc chain and the second polypeptide may comprises a second Fc chain. The first Fc chain and the second Fc chain may each comprise one or more amino acid modifications that promote the association of the first Fc chain with the second Fc chain.

In some aspects, the first Fc chain comprises a first CH3 domain, and the second Fc chain comprises a second CH3 domain, and the first CH3 domain and the second CH3 domain each comprise a different and complementary sequence, and the different and complementary sequences are selected from one of the following pairs of different and complementary sequences: (i) SEQ ID NO: 106 and SEQ ID NO:111; (ii) SEQ ID NO:147 and SEQ ID NO: 148; and (iii) SEQ ID NO: 124, and SEQ ID NO:127.

The disclosure provides an IL-4/IL-13/IL-33 antibody, wherein the identity of the first, second, third, fourth, and fifth polypeptide chains is selected from the group consisting of
(i) the first polypeptide chain comprises SEQ ID NO: 146, the second polypeptide chain comprises SEQ ID NO: 145, the third polypeptide chain comprises SEQ ID NO: 109, the fourth polypeptide chain comprises SEQ ID NO: 196, and the fifth polypeptide chain comprises SEQ ID NO: 103;
(ii) the first polypeptide chain comprises SEQ ID NO: 112, the second polypeptide chain comprises SEQ ID NO: 107, the third polypeptide chain comprises SEQ ID NO: 196, the fourth polypeptide chain comprises SEQ ID NO: 109, and the fifth polypeptide chain comprises SEQ ID NO: 103;
(iii) the first polypeptide chain comprises SEQ ID NO: 118, the second polypeptide chain comprises SEQ ID NO 115, the third polypeptide chain comprises SEQ ID NO: 119, the fourth polypeptide chain comprises SEQ ID NO: 116, and the fifth polypeptide chain comprises SEQ ID NO: 103;
(iv) the first polypeptide chain comprises SEQ ID NO: 118, the second polypeptide chain comprises SEQ ID NO: 115, the third polypeptide chain comprises SEQ ID NO: 120, the fourth polypeptide chain comprises SEQ ID NO: 116, and the fifth polypeptide chain comprises SEQ ID NO: 103;
(v) the first polypeptide chain comprises SEQ ID NO: 209, the second polypeptide chain comprises SEQ ID NO: 121, the third polypeptide chain comprises SEQ ID NO: 196, the fourth polypeptide chain comprises SEQ ID NO: 109, and the fifth polypeptide chain comprises SEQ ID NO: 103;
(vi) the first polypeptide chain comprises SEQ ID NO: 128, the second polypeptide chain comprises SEQ ID NO: 125, the third polypeptide chain comprises SEQ ID NO: 79, the fourth polypeptide chain comprises SEQ ID NO: 208, and the fifth polypeptide chain comprises SEQ ID NO: 122;
(vii) the first polypeptide chain comprises SEQ ID NO: 132, the second polypeptide chain comprises SEQ ID NO: 130, the third polypeptide chain comprises SEQ ID NO: 79, the fourth polypeptide chain comprises SEQ ID NO: 27, and the fifth polypeptide chain comprises SEQ ID NO: 122;
(viii) the first polypeptide chain comprises SEQ ID NO: 134, the second polypeptide chain comprises SEQ ID NO: 133, the third polypeptide chain comprises SEQ ID NO: 79, the fourth polypeptide chain comprises SEQ ID NO: 27, and the fifth polypeptide chain comprises SEQ ID NO: 122;
(ix) the first polypeptide chain comprises SEQ ID NO: 121, the second polypeptide chain comprises SEQ ID NO: 144, the third polypeptide chain comprises SEQ ID NO: 196, the fourth polypeptide chain comprises SEQ ID NO: 136, and the fifth polypeptide chain comprises SEQ ID NO: 143;
(x) the first polypeptide chain comprises SEQ ID NO: 137, the second polypeptide chain comprises SEQ ID NO: 135, the third polypeptide chain comprises SEQ ID NO: 138, the fourth polypeptide chain comprises SEQ ID NO: 136, and the fifth polypeptide chain comprises SEQ ID NO: 122;
(xi) the first polypeptide chain comprises SEQ ID NO: 142, the second polypeptide chain comprises SEQ ID NO: 140, the third polypeptide chain comprises SEQ ID NO: 79, the fourth polypeptide chain comprises SEQ ID NO: 27, and the fifth polypeptide chain comprises SEQ ID NO: 122.

In some aspects, the disclosure provided an isolated antibody that specifically binds IL-33, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an IL-33 binding site, and
wherein the first polypeptide chain comprises SEQ ID NO: 132, the second polypeptide chain comprises SEQ ID NO: 130, the third polypeptide chain comprises SEQ ID NO: 79, the fourth polypeptide chain comprises SEQ ID NO: 27, and the fifth polypeptide chain comprises SEQ ID NO: 122.

The anti-IL-4/IL-13/IL-33 antibodies of the present disclosure demonstrate a combination of improved anti-IL-4/IL-13/IL-33 activity while minimizing an increase in viscosity, relative to parental antibodies. Anti-IL-4/IL-13/IL-33 antibodies of the present disclosure may have a viscosity of less than 20 cP at concentrations of at least 50 mg/mL in a buffer of 20 mM Histidine, 8.5% sucrose, 0.05 mg/mL EDTA pH 6.0. Anti-IL-4/IL-13/IL-33 antibodies of the present disclosure may have a viscosity of less than 20 cP at concentrations of at least 70 mg/mL in a buffer of 20 mM Histidine, 8.5% sucrose, 0.05 mg/mL EDTA pH 6.0. Anti-IL-4/IL-13/IL-33 antibodies of the present disclosure may have a viscosity of less than 80 cP at concentrations of at least 90 mg/m in a buffer of 20 mM Histidine, 8.5% sucrose, 0.05 mg/mL EDTA pH 6.0.L. Anti-IL-4/IL-13/IL-33 antibodies of the present disclosure may have a viscosity of less than 15 cP at concentrations of at least 50 mg/mL in a buffer of 20 mM Histidine, 8.5% sucrose, 0.05 mg/mL EDTA pH 6.0. Anti-IL-4/IL-13/IL-33 antibodies of the present disclosure may have a viscosity of less than 15 cP at concentrations of at least 70 mg/mL in a buffer of 20 mM Histidine, 8.5% sucrose, 0.05 mg/mL EDTA pH 6.0. Anti-IL-4/IL-13/IL-33 antibodies of the present disclosure may have a viscosity of less than 15 cP at concentrations of at least 80 mg/mL in a buffer of 20 mM Histidine, 8.5% sucrose, 0.05 mg/mL EDTA pH 6.0. Anti-IL-4/IL-13/IL-33 antibodies of the present disclosure may have a viscosity of less than 15 cP at concentrations of at least 90 mg/mL in a buffer of 20 mM Histidine, 8.5% sucrose, 0.05 mg/mL EDTA pH 6.0. Anti-IL-4/IL-13/IL-33 antibodies of the present disclosure may have a viscosity of less than 15 cP at concentrations of at least 90 mg/mL in a buffer of 20 mM Histidine, 8.5% sucrose, 0.05 mg/mL EDTA pH 6.0. Anti-IL-4/IL-13/IL-33 antibodies of the present disclosure may have a viscosity of less than 13 cP at concentrations of at least 90 mg/mL in a buffer of 20 mM Histidine, 8.5% sucrose, 0.05 mg/mL EDTA pH 6.0. Anti-IL-4/IL-13/IL-33 antibodies of the present disclosure may have a viscosity of less than 13 cP at concentrations of at least 94 mg/mL in a buffer of 20 mM Histidine, 8.5% sucrose, 0.05 mg/mL EDTA pH 6.0.

Anti-IL-4/IL-13/IL-33 antibodies of the present disclosure may have a terminal half-life of at least 16 days in TG32 mice. Anti-IL-4/IL-13/IL-33 antibodies of the present disclosure may have a terminal half-life of at least 12 days in cynomolgus monkeys.

Anti-IL-4/IL-13/IL-33 antibodies of the disclosure may bind to human IL-4 with a binding affinity of less than 220 nM, as measured by SPR. Favorably, Anti-IL-4/IL-13/IL-33 antibodies do not specifically bind to mouse or rat IL-4.

Anti-IL-4/IL-13/IL-33 antibodies of the disclosure may bind to human IL-4 with a binding affinity of less than 1 pM, as measured by KinExA in a fixed antigen assay in PBS. Anti-IL-4/IL-13/IL-33 antibodies of the disclosure may bind to cynomolgus IL-4 with a binding affinity of less than 5 pM, as measured by KinExA in a fixed antigen assay in PBS. Anti-IL-4/IL-13/IL-33 antibodies of the disclosure may bind to human IL-4 with a binding affinity of less than 0.5 pM, as measured by KinExA in a fixed antigen assay in PBS. Anti-IL-4/IL-13/IL-33 antibodies of the disclosure may bind to cynomolgus IL-13 with a binding affinity of less than 3 pM, as measured by KinExA in a fixed antigen assay in PBS.

Anti-IL-4/IL-13/IL-33 antibodies of the disclosure may bind to human IL-13 with a binding affinity of less than 220 nM, as measured by SPR. Favorably, Anti-IL-4/IL-13/IL-33 antibodies do not specifically bind to mouse, rat, or rabbit IL-13.

Anti-IL-4/IL-13/IL-33 antibodies of the disclosure may bind to human IL-13 with a binding affinity of less than 1 pM, as measured by KinExA in a fixed antigen assay in PBS. Anti-IL-4/IL-13/IL-33 antibodies of the disclosure may bind to cynomolgus IL-13 with a binding affinity of less than 1 pM, as measured by KinExA in a fixed antigen assay in PBS.

Anti-IL-4/IL-13/IL-33 antibodies of the disclosure may bind to human IL-13 with a binding affinity of less than 15 pM, as measured by KinExA in peripheral blood monocytes. Anti-IL-4/IL-13/IL-33 antibodies of the disclosure may bind to cynomolgus IL-13 with a binding affinity of less than 55 pM, as measured by KinExA in peripheral blood monocytes. Anti-IL-4/IL-13/IL-33 antibodies of the disclosure may bind to human IL-13 with a binding affinity of less than 20 pM, as measured by KinExA in human whole blood. Anti-IL-4/IL-13/IL-33 antibodies of the disclosure may bind to cynomolgus IL-13 with a binding affinity of less than 55 pM, as measured by KinExA in human whole blood.

Anti-IL-4/IL-13/IL-33 antibodies of the present disclosure may be characterized by one or more of (i) an IC₅₀ of less than 70 nM in a human monocyte assay for neutralization of IL-4 induction of CD23; (ii) an IC₅₀ of less than 70 nM in an a human monocyte assay for neutralization of IL-13 induction of CD23; (iii) an IC₅₀ of less than 30 nM in a wild-type IL-33 neutralization HEK-Blue SEAP assay, and (iv) an IC₅₀ of less than 260 nM in a recombinant constitutively active IL-33 (mm2) neutralization HEK-Blue SEAP assay. Anti-IL-4/IL-13/IL-33 antibodies of the present disclosure may be characterized by one or more of (i) an IC₅₀ of less than 20 nM in a human monocyte assay for neutralization of IL-4 induction of CD23; (ii) an IC₅₀ of less than 20 nM in a human monocyte assay for neutralization of IL-13 induction of CD23; and (iii) an IC₅₀ of less than 30 nM in a wild-type IL-33 neutralization HEK-Blue SEAP assay. Anti-IL-4/IL-13/IL-33 antibodies of the present disclosure may be characterized by one or more of (i) an IC₅₀ of less than 20 nM in a human monocyte assay for neutralization of IL-4 induction of CD23; (ii) an IC₅₀ of less than 20 nM in a human monocyte assay for neutralization of IL-13 induction of CD23; and (iii) an IC₅₀ of less than 30 nM in a wild-type IL-33 neutralization HEK-Blue SEAP assay.

Anti-IL-4/IL-13/IL-33 antibodies of the present disclosure may be characterized by an IC₅₀ of less than 10 pM in a human monocyte assay for neutralization of IL-4 induction of CD23. Anti-IL-4/IL-13/IL-33 antibodies of the present disclosure may be characterized by an IC₅₀ of less than 15 pM in a human monocyte assay for neutralization of cynomolgus monkey IL-4 induction of CD23. Anti-IL-4/IL-13/IL-33 antibodies of the present disclosure may be characterized by an IC₅₀ of less than 25 pM in a human monocyte assay for neutralization of IL-4 induction of CD23. Anti-IL-4/IL-13/IL-33 antibodies of the present disclosure may be characterized by an IC₅₀ of less than 35 pM in a human monocyte assay for neutralization of cynomolgus monkey IL-4 induction of CD23.

Anti-IL-4/IL-13/IL-33 antibodies of the present disclosure may be characterized by an IC₅₀ of less than 15 pM in a human monocyte assay for neutralization of IL-13 induction of CD23. Anti-IL-4/IL-13/IL-33 antibodies of the present disclosure may be characterized by an IC₅₀ of less than 12 pM in a human monocyte assay for neutralization of IL-13 induction of CD23. IL-4 neutralization and IL-13 neutralization may each be determined by flow cytometry determination of CD23 positive cells following incubation of gated monocytes from IL-4- or IL-13-stimulated human peripheral blood mononuclear cells with the antibody.

Anti-IL-4/IL-13/IL-33 antibodies of the present disclosure may be characterized by an IC₅₀ of less than 15 pM in an IL-33 neutralization HEK-Blue SEAP assay. Anti-IL-4/IL-13/IL-33 antibodies of the present disclosure may be characterized by an IC₅₀ of less than 15 pM in an IL-33 neutralization assay of IFNγ induction in human whole blood.

In some aspects, the disclosure provides an isolated anti-IL-4/IL-13/IL-33 antibody that specifically bind IL-33 through an IL-33 heavy chain variable region (IL33-VH) and an IL-33 light chain variable region (IL33-VL); that specifically binds IL-4 through an IL-4 heavy chain variable region (IL4-VH) and an IL-4 light chain variable region (IL4-VL); and that specifically binds IL-13 through an IL-13 heavy chain variable region (IL13-VH) and an IL-13 light chain variable region (IL33-VL); wherein the IL33-VH comprises the CDR-H1, CDR-H2, and CDR-H3 sequences encoded by the plasmid deposited at the ATCC and having ATCC Accession No. PTA-127210, and the IL33-VL comprises the CDR-L1, CDR-L2, and CDR-L3 sequences encoded by the plasmid deposited at the ATCC and having ATCC Accession No. PTA-127209, and the IL4-VH comprises the CDR-H1, CDR-H2, and CDR-H3 sequences encoded by the plasmid deposited at the ATCC and having ATCC Accession No. PTA-127198, and the IL4-VL comprises the CDR-L1, CDR-L2, and CDR-L3 sequences encoded by the plasmid deposited at the ATCC and having ATCC Accession No. PTA-127197, and the IL13-VH comprises the CDR-H1, CDR-H2, and CDR-H3 sequences encoded by the plasmid deposited at the ATCC and having ATCC Accession No. PTA-127196, and the IL13-VL comprises the CDR-L1, CDR-L2, and CDR-L3 sequences encoded by the plasmid deposited at the ATCC and having ATCC Accession No. PTA-127195.

In some aspects, the disclosure provides anti-IL-4/IL-13/IL-33 antibodies comprising an IL33-VH sequence encoded by the plasmid deposited at the ATCC and having ATCC Accession No. PTA-127210; an IL33-VL sequence encoded by the plasmid deposited at the ATCC and having ATCC Accession No. PTA-127209; an IL4-VH sequence encoded by the plasmid deposited at the ATCC and having ATCC Accession No. PTA-127198; an IL4-VL sequence encoded by the plasmid deposited at the ATCC and having ATCC Accession No. PTA-127197; an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

In some aspects, the disclosure provides anti-IL-4/IL-13/IL-33 antibodies comprising the sequence encoded by the plasmid deposited at the ATCC and having ATCC Accession No. PTA-127208; sequence encoded by the plasmid deposited at the ATCC and having ATCC Accession No. PTA-127192; the sequence encoded by the plasmid deposited at the ATCC and having ATCC Accession No. PTA-127207; the sequence encoded by the plasmid deposited at the ATCC and having ATCC Accession No. PTA-127194; and the sequence encoded by the plasmid deposited at the ATCC and having ATCC Accession No. PTA-127193.

### Antibodies to IL-4/IL-13/TSLP

The innate alarmin TSLP is elevated in AD and promotes type 2 immune responses at the barrier surfaces (41, 42). TSLP is produced by epithelial cells, mast cells, keratinocytes, and fibroblasts. A short form is maintained under homeostatic conditions, while the long form, targeted by IL413TSLP-1024, is associated with inflammation (44). Upon cellular damage, TSLP is released and binds to a heterodimeric receptor comprised of TSLPR and IL 7Rα on a range of immune cell types (4), resulting in activation of DCs and monocytes, production of type 2 cytokines, and escalation of Th2 effector responses (64). TSLP is an upstream regulator of type 2 cytokines, but also triggers a distinct signaling pathway linked to STAT5, the blockade of which may provide separate and additional therapeutic activity (64).

An anti-TSLP mAb, Tezspire™ (Tezepelumab; Amgen/AstraZeneca) is approved for the treatment of severe asthma, where it has shown strong efficacy, significantly reducing exacerbation rates over placebo (65). Importantly, this result was independent of baseline blood eosinophil count (65), suggesting that efficacy of TSLP blockade may extend beyond the type 2 disease profile targeted by Dupixent®. In AD, tezepelumab displayed positive trends, but limited signs of efficacy as a standalone therapy (47).

At the barrier surfaces, epithelial damage releases alarmins TSLP and IL-33, which promote a cascade of downstream effector responses, including local generation of IL-4, IL-5, and IL-13. In turn, these cytokines promote and sustain epithelial dysfunction, resulting in an escalating cycle of tissue damage, inflammation, fibrosis, and itch (41, 69, 74).

IL413TSLP-1024 and other IL-4/IL-13/TSLP antibodies were designed to achieve combinatorial blockade of three distinct, clinically validated pathways for treatment of atopic disorders. IL413TSLP-1024 in particular neutralizes IL-4 and IL-13 with approximately 10-fold greater potency than Dupixent®. IL413TSLP-1024 includes a TSLP binding domain with activity comparable to Tezepelumab, and has the potential to extend the efficacy of IL-4/13 blockade to disease endotypes beyond the type 2 profile.

The disclosure provides antibodies that bind to IL-4, IL-13, and TSLP. As used herein, the terms IL-4, IL-13, and TSLP include variants, isoforms, homologs, orthologs and paralogs of one or more of IL-4, IL-13, and TSLP respectively. In some embodiments, an antibody disclosed herein cross-reacts with one or more of IL-4, IL-13, and TSLP from species other than human, such as IL-4, IL-13, and TSLP of cynomolgus monkey. In some embodiments, an antibody may be completely specific for human IL-4, IL-13, and TSLP and may not exhibit species cross-reactivity or other types of cross-reactivity. As used herein the term IL-4, IL-13, and TSLP refers to naturally occurring human IL-4, IL-13, and TSLP unless contextually dictated otherwise. An “IL-4/IL-13/TSLP antibody” “anti-IL-4/IL-13/TSLP antibody” or other similar designation means any antibody (as defined herein) that binds or reacts with IL-4, IL-13, and TSLP, an isoform, fragment or derivative thereof.

In some embodiments, the invention provides an IL-4/IL-13/TSLP antibody having a light chain variable region (VL) sequence and a heavy chain variable region (VH) sequence as found in Table 84 or variants thereof.

The invention also provides CDR portions of IL-4/IL-13/TSLP antibodies. Determination of CDR regions is defined in Example 1. In some embodiments, the IL-4/IL-13/TSLP antibody comprises the six CDRs of an IL-4 antibody selected from Table 80, the six CDRs of an IL-13 antibody selected from Table 81, and the six CDRs of a TSLP antibody selected from Table 83. In some embodiments, the antibody comprises the VH and VL of an IL-4 antibody selected from Table 80 or Table 84, the VH and VL of an IL-13 antibody selected from Table 81 or Table 84, and the VH and VL of a TSLP antibody selected from Table 83 or Table 84. In some embodiments, the IL-4/IL-13/TSLP antibody comprises sequences selected from Table 84.

In some embodiments, the disclosure provides anti-IL-4/IL-13/TSLP antibodies containing variations of the CDRs, VH, VL, HC, and LC regions shown in one or more of Table 80, 81, 83, 84, and 87, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 80, 81, 83, 84, and 87. These amounts are not meant to be limiting and increments between the recited percentages are specifically envisioned as part of the disclosure.

In some embodiments, the invention provides an IL-4/IL-13/IL-33 antibody having a light chain variable region (VL) sequence and a heavy chain variable region (VH) sequence as found in Table 85, or variants thereof.

The invention also provides CDR portions of IL-4/IL-13/TSLP antibodies. Determination of CDR regions is defined in Example 1. In some embodiments, the IL-4/IL-13/TSLP antibody comprises the six CDRs of an IL-4 antibody selected from one of Table 80 and 84, the six CDRs of an IL-13 antibody selected from one of Table 81 and 84, and the six CDRs of a TSLP antibody selected from one of Table 83 and 84. In some embodiments, the antibody comprises the VH and VL of an IL-4 antibody selected from Table 80 and 84, the VH and VL of an IL-13 antibody selected from Table 81 or Table 84, and the VH and VL of a TSLP antibody selected from Table 83 or Table 84. In some embodiments, the IL-4/IL-13/TSLP antibody comprises sequences selected from Table 84.

In some embodiments, the disclosure provides anti-IL-4/IL-13/TSLP antibodies containing variations of the CDRs, VH, VL, HC, and LC regions shown in one or more of Table 80, 81, 83, 84, and 87, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in one or more of Table 80, 81, 83, 84, and 87. These amounts are not meant to be limiting and increments between the recited percentages are specifically envisioned as part of the disclosure.

The present disclosure relates to an isolated antibody that specifically binds to TSLP, specifically binds to IL-4, and specifically binds to IL-13, comprising an TSLP binding domain, an IL-4 binding domain, and an IL-13 binding domain. The IL-4/IL-13/TSLP antibody may comprise
(i) the TSLP 3 binding domain comprises a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 90; and
(ii) the IL-4 binding domain comprises a heavy chain variable region (IL4-VH) and a light chain variable region (IL4-VL), wherein the CDR-H1 comprises the amino acid sequence of SEQ ID NO: 18; the CDR-H2 comprises the amino acid sequence of SEQ ID NO: 2; the CDR-H3 comprises the amino acid sequence of SEQ ID NO: 3; the CDR-L1 comprises the amino acid sequence of SEQ ID NO: 24; the CDR-L2 comprises the amino acid sequence of SEQ ID NO: 12, and the CDR-L3 comprises the amino acid sequence of SEQ ID NO: 25, and
(iii) the IL-13 binding domain comprises a heavy chain variable region (IL13-VH) and a light chain variable region (IL13-VL), wherein the CDR-H1 comprises the amino acid sequence of SEQ ID NO: 41; the CDR-H2 comprises the amino acid sequence of SEQ ID NO: 42; the CDR-H3 comprises the amino acid sequence of SEQ ID NO:-50; the CDR-L1 comprises the amino acid sequence of SEQ ID NO: 53; the CDR-L2 comprises the amino acid sequence of SEQ ID NO: 37, and the CDR-L3 comprises the amino acid sequence of SEQ ID NO: 38.
   In some aspects of the disclosure, the IL-4/IL-13/TSLP antibody may comprise
(i) a TSLP binding portion comprising the TSLP-VH of SEQ ID NO: 92, and the TSLP-VL of SEQ ID NO: 94;
(ii) a IL-4 binding portion comprising a IL4-VH of SEQ ID NO: 22, and a IL4-VL of SEQ ID NO: 26; and
(iii) a IL-13 binding portion comprising a IL13-VH of SEQ ID NO: 51, and a IL13-VL of SEQ ID NO: 54.
   The TSLP binding domain may be fused to the IL-4 binding domain. The TSLP binding domain may be fused to the IL-13 binding domain. The IL-13 binding domain may be fused to the IL-4 binding domain. The fusion may be with or without a linker. If present, the linker may comprise a sequence of amino acids between 2 and 20 amino acids in length. The linker may consist of one or more amino acids selected from glycine, alanine, and serine. The linker may consist of one or more amino acids selected from glycine and serine. The linker may comprise SEQ ID NO: 104.

IL-4/IL-13/TSLP antibodies of the invention may comprise a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site.

In some aspects, the first antigen binding site specifically binds IL-4, the second antibody binding site specifically binds IL-13, and the third antigen binding site specifically binds TSLP. In some aspects, the first antigen binding site specifically binds IL-4, the second antibody binding site specifically binds TSLP, and the third antigen binding site specifically binds IL-13. In some aspects, the first antigen binding site specifically binds IL-13, the second antibody binding site specifically binds IL-4, and the third antigen binding site specifically binds TSLP. In some aspects, the first antigen binding site specifically binds IL-13, the second antibody binding site specifically binds TSLP, and the third antigen binding site specifically binds IL-4. In some aspects, the first antigen binding site specifically binds TSLP, the second antibody binding site specifically binds IL-13, and the third antigen binding site specifically binds IL-4. In some aspects, the first antigen binding site specifically binds TSLP, the second antibody binding site specifically binds IL-4, and the third antigen binding site specifically binds IL-13.

The IL-4/IL-13/TSLP antibody may comprise one or more domain-swap Fab domains. The domain-swap Fab domains may be selected from the group consisting of TSLP-xFab, IL4-xFab, and IL13-xFab

The IL-4/IL-13/TSLP antibody may comprise a first Fab domain comprising the IL13-Fab, a second Fab domain comprising IL4-Fab, and a third Fab domain comprising the TSLP-Fab.

To facilitate formation of the multispecific antibody, the first polypeptide may comprises a first Fc chain and the second polypeptide may comprises a second Fc chain. The first Fc chain and the second Fc chain may each comprise one or more amino acid modifications that promote the association of the first Fc chain with the second Fc chain.

In some aspects, the first Fc chain comprises a first CH3 domain, and the second Fc chain comprises a second CH3 domain, and the first CH3 domain and the second CH3 domain each comprise a different and complementary sequence, and the different and complementary sequences are selected from one of the following pairs of different and complementary sequences: (i) SEQ ID NO: 106 and SEQ ID NO:111; (ii) SEQ ID NO:147 and SEQ ID NO: 148; and (iii) SEQ ID NO: 124, and SEQ ID NO:127.

The disclosure provides an IL-4/IL-13/TSLP antibody, wherein the identity of the first, second, third, fourth, and fifth polypeptide chains is selected from the group consisting of
(i) the first polypeptide chain comprises SEQ ID NO: 146, the second polypeptide chain comprises SEQ ID NO: 149, the third polypeptide chain comprises SEQ ID NO: 109, the fourth polypeptide chain comprises SEQ ID NO: 196, and the fifth polypeptide chain comprises SEQ ID NO: 150;
(ii) the first polypeptide chain comprises SEQ ID NO: 112, the second polypeptide chain comprises SEQ ID NO: 151, the third polypeptide chain comprises SEQ ID NO: 196, the fourth polypeptide chain comprises SEQ ID NO: 109, and the fifth polypeptide chain comprises SEQ ID NO: 150;
(iii) the first polypeptide chain comprises SEQ ID NO: 112, the second polypeptide chain comprises SEQ ID NO 159, the third polypeptide chain comprises SEQ ID NO: 196, the fourth polypeptide chain comprises SEQ ID NO: 109, and the fifth polypeptide chain comprises SEQ ID NO: 150;
(iv) the first polypeptide chain comprises SEQ ID NO: 161, the second polypeptide chain comprises SEQ ID NO: 162, the third polypeptide chain comprises SEQ ID NO: 98, the fourth polypeptide chain comprises SEQ ID NO: 197, and the fifth polypeptide chain comprises SEQ ID NO: 163;
(v) the first polypeptide chain comprises SEQ ID NO: 146, the second polypeptide chain comprises SEQ ID NO: 154, the third polypeptide chain comprises SEQ ID NO: 109, the fourth polypeptide chain comprises SEQ ID NO: 196, and the fifth polypeptide chain comprises SEQ ID NO: 155;
(vi) the first polypeptide chain comprises SEQ ID NO: 112, the second polypeptide chain comprises SEQ ID NO: 156, the third polypeptide chain comprises SEQ ID NO: 196, the fourth polypeptide chain comprises SEQ ID NO: 109, and the fifth polypeptide chain comprises SEQ ID NO: 155;
(vii) the first polypeptide chain comprises SEQ ID NO: 146, the second polypeptide chain comprises SEQ ID NO: 152, the third polypeptide chain comprises SEQ ID NO: 109, the fourth polypeptide chain comprises SEQ ID NO: 196, and the fifth polypeptide chain comprises SEQ ID NO: 98;
(viii) the first polypeptide chain comprises SEQ ID NO: 112, the second polypeptide chain comprises SEQ ID NO: 153, the third polypeptide chain comprises SEQ ID NO: 196, the fourth polypeptide chain comprises SEQ ID NO: 109, and the fifth polypeptide chain comprises SEQ ID NO: 98;
(ix) the first polypeptide chain comprises SEQ ID NO: 112, the second polypeptide chain comprises SEQ ID NO: 157, the third polypeptide chain comprises SEQ ID NO: 196, the fourth polypeptide chain comprises SEQ ID NO: 109, and the fifth polypeptide chain comprises SEQ ID NO: 158;
(x) the first polypeptide chain comprises SEQ ID NO: 146, the second polypeptide chain comprises SEQ ID NO: 160, the third polypeptide chain comprises SEQ ID NO: 109, the fourth polypeptide chain comprises SEQ ID NO: 196, and the fifth polypeptide chain comprises SEQ ID NO: 158;
(xi) the first polypeptide chain comprises SEQ ID NO: 161, the second polypeptide chain comprises SEQ ID NO: 164, the third polypeptide chain comprises SEQ ID NO: 98, the fourth polypeptide chain comprises SEQ ID NO: 197, and the fifth polypeptide chain comprises SEQ ID NO: 122;
(xii) the first polypeptide chain comprises SEQ ID NO: 165, the second polypeptide chain comprises SEQ ID NO: 130, the third polypeptide chain comprises SEQ ID NO: 99, the fourth polypeptide chain comprises SEQ ID NO: 27, and the fifth polypeptide chain comprises SEQ ID NO: 122;
(xiii) the first polypeptide chain comprises SEQ ID NO: 165, the second polypeptide chain comprises SEQ ID NO: 130, the third polypeptide chain comprises SEQ ID NO: 215, the fourth polypeptide chain comprises SEQ ID NO: 27, and the fifth polypeptide chain comprises SEQ ID NO: 122; and
(xiv) the first polypeptide chain comprises SEQ ID NO: 165, the second polypeptide chain comprises SEQ ID NO: 130, the third polypeptide chain comprises SEQ ID NO: 216, the fourth polypeptide chain comprises SEQ ID NO: 27, and the fifth polypeptide chain comprises SEQ ID NO: 122.

In some aspects, the disclosure provided an isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a TSLP binding site; and
wherein the first polypeptide chain comprises SEQ ID NO: 165, the second polypeptide chain comprises SEQ ID NO: 130, the third polypeptide chain comprises SEQ ID NO: 99, the fourth polypeptide chain comprises SEQ ID NO: 27, and the fifth polypeptide chain comprises SEQ ID NO: 122.
   In some aspects, the disclosure provided an isolated antibody that specifically binds TSLP, that specifically binds to IL-4, and that specifically binds to IL-13, wherein
(i) the TSLP binding domain comprises a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 211; and
(ii) the IL-4 binding domain comprises a heavy chain variable region (IL4-VH) and a light chain variable region (IL4-VL), wherein the CDR-H1 comprises the amino acid sequence of SEQ ID NO: 18; the CDR-H2 comprises the amino acid sequence of SEQ ID NO: 2; the CDR-H3 comprises the amino acid sequence of SEQ ID NO: 3; the CDR-L1 comprises the amino acid sequence of SEQ ID NO: 24; the CDR-L2 comprises the amino acid sequence of SEQ ID NO: 12, and the CDR-L3 comprises the amino acid sequence of SEQ ID NO: 25, and
(iii) the IL-13 binding domain comprises a heavy chain variable region (IL13-VH) and a light chain variable region (IL13-VL), wherein the CDR-H1 comprises the amino acid sequence of SEQ ID NO: 41; the CDR-H2 comprises the amino acid sequence of SEQ ID NO: 42; the CDR-H3 comprises the amino acid sequence of SEQ ID NO:-50; the CDR-L1 comprises the amino acid sequence of SEQ ID NO: 53; the CDR-L2 comprises the amino acid sequence of SEQ ID NO: 37, and the CDR-L3 comprises the amino acid sequence of SEQ ID NO: 38.
   In some aspects, the disclosure provided an isolated antibody that specifically binds TSLP, that specifically binds to IL-4, and that specifically binds to IL-13, wherein
(i) the TSLP binding portion comprises the TSLP-VH of SEQ ID NO: 92, and the TSLP-VL of SEQ ID NO: 213;
(ii) the IL-4 binding portion comprises a IL4-VH of SEQ ID NO: 22, and a IL4-VL of SEQ ID NO: 26; and
(iii) the IL-13 binding portion comprises the IL13-VH of SEQ ID NO: 51, and the IL13-VL of SEQ ID NO: 54.
   In some aspects, the disclosure provided an isolated antibody that specifically binds TSLP, that specifically binds to IL-4, and that specifically binds to IL-13, wherein
(i) the TSLP binding domain comprises a TSLP-VH sequence encoded by the plasmid deposited at the ATCC and having ATCC Accession No. PTA-127200, and a TSLP-VL sequence encoded by the plasmid deposited at the ATCC and having ATCC Accession No. PTA-______.
(ii) the IL-4 binding domain comprises IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) the IL-13 binding domain comprises an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.
   In some aspects, the disclosure provided an isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an TSLP binding site; and
wherein the first polypeptide chain comprises SEQ ID NO: 165, the second polypeptide chain comprises SEQ ID NO: 130, the third polypeptide chain comprises SEQ ID NO: 215, the fourth polypeptide chain comprises SEQ ID NO: 27, and the fifth polypeptide chain comprises SEQ ID NO: 122.
   In some aspects, the disclosure provided an isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a TSLP binding site, and
wherein the first polypeptide chain comprises a sequence encoded by the plasmid deposited at the ATCC and having ATCC Accession No. PTA-127202; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-______; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.
   In some aspects, the disclosure provided an isolated antibody that specifically binds TSLP, that specifically binds to IL-4, and that specifically binds to IL-13, wherein
(i) the TSLP binding domain comprises a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 212; and
(ii) the IL-4 binding domain comprises a heavy chain variable region (IL4-VH) and a light chain variable region (IL4-VL), wherein the CDR-H1 comprises the amino acid sequence of SEQ ID NO: 18; the CDR-H2 comprises the amino acid sequence of SEQ ID NO: 2; the CDR-H3 comprises the amino acid sequence of SEQ ID NO: 3; the CDR-L1 comprises the amino acid sequence of SEQ ID NO: 24; the CDR-L2 comprises the amino acid sequence of SEQ ID NO: 12, and the CDR-L3 comprises the amino acid sequence of SEQ ID NO: 25, and
(iii) the IL-13 binding domain comprises a heavy chain variable region (IL13-VH) and a light chain variable region (IL13-VL), wherein the CDR-H1 comprises the amino acid sequence of SEQ ID NO: 41; the CDR-H2 comprises the amino acid sequence of SEQ ID NO: 42; the CDR-H3 comprises the amino acid sequence of SEQ ID NO:-50; the CDR-L1 comprises the amino acid sequence of SEQ ID NO: 53; the CDR-L2 comprises the amino acid sequence of SEQ ID NO: 37, and the CDR-L3 comprises the amino acid sequence of SEQ ID NO: 38.
   In some aspects, the disclosure provided an isolated antibody that specifically binds TSLP, that specifically binds to IL-4, and that specifically binds to IL-13, wherein
(i) the TSLP binding portion comprises the TSLP-VH of SEQ ID NO: 92, and the TSLP-VL of SEQ ID NO: 214;
(ii) the IL-4 binding portion comprises a IL4-VH of SEQ ID NO: 22, and a IL4-VL of SEQ ID NO: 26; and
(iii) the IL-13 binding portion comprises the IL13-VH of SEQ ID NO: 51, and the IL13-VL of SEQ ID NO: 54.
   In some aspects, the disclosure provided an isolated antibody that specifically binds TSLP, that specifically binds to IL-4, and that specifically binds to IL-13, wherein
(i) the TSLP binding domain comprises a TSLP-VH sequence encoded by the plasmid deposited at the ATCC and having ATCC Accession No. PTA-127200, and a TSLP-VL sequence encoded by the plasmid deposited at the ATCC and having ATCC Accession No. PTA0-______.
(ii) the IL-4 binding domain comprises IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) the IL-13 binding domain comprises an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.
   In some aspects, the disclosure provided an isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an TSLP binding site; and
wherein the first polypeptide chain comprises SEQ ID NO: 165, the second polypeptide chain comprises SEQ ID NO: 130, the third polypeptide chain comprises SEQ ID NO: 216, the fourth polypeptide chain comprises SEQ ID NO: 27, and the fifth polypeptide chain comprises SEQ ID NO: 122.
   In some aspects, the disclosure provided an isolated antibody that specifically binds TSLP, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a TSLP binding site, and
wherein the first polypeptide chain comprises a sequence encoded by the plasmid deposited at the ATCC and having ATCC Accession No. PTA-127202; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-______; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

Anti-IL-4/IL-13/TSLP antibodies of the present disclosure may have a terminal half-life of at least 14 days in cynomolgus monkeys. Anti-IL-4/IL-13 TSLP antibodies of the present disclosure may have a terminal half-life of at least 18 days in TG32 mice.

Anti-IL-4/IL-13/TSLP antibodies of the present disclosure may have a viscosity of 20 cP at concentrations of at least 100 mg/mL in a buffer of 20 mM Histidine, 8.5% sucrose, 0.05 mg/mL EDTA pH 6.0. Anti-IL-4/IL-13/TSLP antibodies of the present disclosure may have a viscosity of 20 cP at concentrations of at least 110 mg/mL in a buffer of 20 mM Histidine, 8.5% sucrose, 0.05 mg/mL EDTA pH 6.0. Anti-IL-4/IL-13/TSLP antibodies of the present disclosure may have a viscosity of 20 cP at concentrations of at least 120 mg/mL in a buffer of 20 mM Histidine, 8.5% sucrose, 0.05 mg/mL EDTA pH 6.0.

Anti IL-4/IL-13/TSLP antibodies of the disclosure and compositions thereof may be characterized by a score of less than 2% high molecular mass species when determined by analytical size-exclusion chromatography (aSEC). Compositions comprising anti IL-4/IL-13/TSLP antibodies of the invention may be characterized by a score of less than 1% high molecular mass species when determined by analytical size-exclusion chromatography (aSEC). Compositions comprising anti IL-4/IL-13/TSLP antibodies of the invention may be characterized by a score of less than 0.2% high molecular mass species when determined by analytical size-exclusion chromatography (aSEC).

Anti IL-4/IL-13/TSLP antibodies of the disclosure and compositions thereof may be characterized by a score of less than 12 in an affinity-capture self-interaction nanoparticle spectroscopy (AC SINS) assay. Anti IL-4/IL-13/TSLP antibodies of the disclosure and compositions thereof may be characterized by a score of less than 10 in an affinity-capture self-interaction nanoparticle spectroscopy (AC SINS) assay.

Anti-IL-4/IL-13/TSLP antibodies of the disclosure may bind to human IL-4 with a binding affinity of less than 1 pM, as measured by KinExA in a fixed antigen assay in PBS. Anti-IL-4/IL-13/TSLP antibodies of the disclosure may bind to cynomolgus IL-4 with a binding affinity of less than 1 pM, as measured by KinExA in a fixed antigen assay in PBS. Anti-IL-4/IL-13/TSLP antibodies of the disclosure may bind to human IL-4 with a binding affinity of less than 0.5 pM, as measured by KinExA in a fixed antigen assay in PBS.

Anti-IL-4/IL-13/TSLP antibodies of the disclosure may bind to human IL-13 with a binding affinity of less than 1 pM, as measured by KinExA in a fixed antigen assay in PBS. Anti-IL-4/IL-13/TSLP antibodies of the disclosure may bind to human IL-13 with a binding affinity of less than 0.3 pM, as measured by KinExA in a fixed antigen assay in PBS. Anti-IL-4/IL-13/TSLP antibodies of the disclosure may bind to cynomolgus IL-13 with a binding affinity of less than 1 pM, as measured by KinExA in a fixed antigen assay in PBS.

Anti-IL-4/IL-13/TSLP antibodies of the disclosure may bind to human TSLP with a binding affinity of less than 5 pM, as measured by KinExA in a fixed antigen assay in PBS. Anti-IL-4/IL-13/TSLP antibodies of the disclosure may bind to cynomolgus IL-13 with a binding affinity of less than 20 pM, as measured by KinExA in a fixed antigen assay in PBS.

Anti-IL-4/IL-13/TSLP antibodies of the disclosure may bind to human IL-13 with a binding affinity of less than 320 nM, as measured by SPR. Favorably, Anti-IL-4/IL-13/TSLP antibodies do not specifically bind to mouse, rat, or rabbit IL-13.

Anti-IL-4/IL-13/TSLP antibodies of the present disclosure may be characterized by an IC₅₀ of less than 10 pM in a human monocyte assay for neutralization of IL-4 induction of CD23. Anti-IL-4/IL-13/TSLP antibodies of the present disclosure may be characterized by an IC₅₀ of less than 8 pM in a human monocyte assay for neutralization of IL-4 induction of CD23. Anti-IL-4/IL-13/TSLP antibodies of the present disclosure may be characterized by an IC₅₀ of less than 15 pM in a human monocyte assay for neutralization of IL-13 induction of CD23. Anti-IL-4/IL-13/TSLP antibodies of the present disclosure may be characterized by an IC₅₀ of less than 15 pM in a human monocyte assay for neutralization of IL-13 induction of CD23. IL-4 neutralization and IL-13 neutralization may each be determined by flow cytometry determination of CD23 positive cells following incubation of gated monocytes from IL-4- or IL-13-stimulated human peripheral blood mononuclear cells with the antibody.

Anti-IL-4/IL-13/TSLP antibodies of the present disclosure may be characterized by an IC₅₀ of less than 25 pM in a human monocyte assay for neutralization of IL-4 induction of CD23. Anti-IL-4/IL-13/TSLP antibodies of the present disclosure may be characterized by an IC₅₀ of less than 10 pM in a human monocyte assay for neutralization of IL-4 induction of CD23. Anti-IL-4/IL-13/TSLP antibodies of the present disclosure may be characterized by an IC₅₀ of less than 8 pM in a human monocyte assay for neutralization of IL-4 induction of CD23. Anti-IL-4/IL-13/TSLP antibodies of the present disclosure may be characterized by an IC₅₀ of less than 20 pM in a human monocyte assay for neutralization of cynomolgus monkey IL-4 induction of CD23. Anti-IL-4/IL-13/TSLP antibodies of the present disclosure may be characterized by an IC₅₀ of less than 15 pM in a human monocyte assay for neutralization of cynomolgus monkey IL-4 induction of CD23. Anti-IL-4/IL-13/TSLP antibodies of the present disclosure may be characterized by an IC₅₀ of less than 11 pM in a human monocyte assay for neutralization of cynomolgus monkey IL-4 induction of CD23. IL-4 neutralization may be determined by flow cytometry determination of CD23 positive cells.

Anti-IL-4/IL-13/TSLP antibodies of the present disclosure may be characterized by an IC₅₀ of less than 15 pM in a human monocyte assay for neutralization of IL-13 induction of CD23. Anti-IL-4/IL-13/TSLP antibodies of the present disclosure may be characterized by an IC₅₀ of less than 12 pM in a human monocyte assay for neutralization of IL-13 induction of CD23. Anti-IL-4/IL-13/TSLP antibodies of the present disclosure may be characterized by an IC₅₀ of less than 12 pM in a human whole blood assay for neutralization of IL-13 induction of CD23. Anti-IL-4/IL-13/TSLP antibodies of the present disclosure may be characterized by an IC₅₀ of less than 60 pM in a human monocyte assay for neutralization of cynomolgus monkey IL-13 induction of CD23. Anti-IL-4/IL-13/TSLP antibodies of the present disclosure may be characterized by an IC₅₀ of less than 55 pM in a human monocyte assay for neutralization of cynomolgus monkey IL-13 induction of CD23. Anti-IL-4/IL-13/TSLP antibodies of the present disclosure may be characterized by an IC₅₀ of less than 45 pM in a human whole blood assay for neutralization of cynomolgus monkey IL-13 induction of CD23. IL-13 neutralization may be determined by flow cytometry determination of CD23 positive cells.

Anti-IL-4/IL-13/TSLP antibodies of the present disclosure may be characterized by an IC₅₀ of less than 15 pM in a human TSLP neutralization assay. Anti-IL-4/IL-13/TSLP antibodies of the present disclosure may be characterized by an IC₅₀ of less than 35 pM in a cynomolgus TSLP neutralization assay. TSLP neutralization may be determined by flow cytometry determination of TARC production in human primary PBMCs. Anti-IL-4/IL-13/TSLP antibodies of the present disclosure may be characterized by an IC₅₀ of less than 10 pM in a human TSLP neutralization assay in human whole blood. Anti-IL-4/IL-13/TSLP antibodies of the present disclosure may be characterized by an IC₅₀ of less than 35 pM in a cynomolgus TSLP neutralization assay in human whole blood. TSLP neutralization may be determined by flow cytometry determination of TARC production in human primary PBMCs.

In some aspects, the disclosure provides an isolated anti-IL-4/IL-13/TSLP3 antibody that specifically bind TSLP through an TSLP heavy chain variable region (TSLP-VH) and an TSLP light chain variable region (TSLP-VL); that specifically binds IL-4 through an IL-4 heavy chain variable region (IL4-VH) and an IL-4 light chain variable region (IL4-VL); and that specifically binds IL-13 through an IL-13 heavy chain variable region (IL13-VH) and an IL-13 light chain variable region (IL33-VL); wherein the TSLP-VH comprises the CDR-H1, CDR-H2, and CDR-H3 sequences encoded by the plasmid deposited at the ATCC and having ATCC Accession No. PTA-127200, and the TSLP-VL comprises the CDR-L1, CDR-L2, and CDR-L3 sequences encoded by the plasmid deposited at the ATCC and having ATCC Accession No. PTA-127199, and the IL4-VH comprises the CDR-H1, CDR-H2, and CDR-H3 sequences encoded by the plasmid deposited at the ATCC and having ATCC Accession No. PTA-127198, and the IL4-VL comprises the CDR-L1, CDR-L2, and CDR-L3 sequences encoded by the plasmid deposited at the ATCC and having ATCC Accession No. PTA-127197, and the IL13-VH comprises the CDR-H1, CDR-H2, and CDR-H3 sequences encoded by the plasmid deposited at the ATCC and having ATCC Accession No. PTA-127196, and the IL13-VL comprises the CDR-L1, CDR-L2, and CDR-L3 sequences encoded by the plasmid deposited at the ATCC and having ATCC Accession No. PTA-127195.

In some aspects, the disclosure provides anti-IL-4/IL-13/TSLP antibodies comprising a TSLP-VH sequence encoded by the plasmid deposited at the ATCC and having ATCC Accession No. PTA-127200, a TSLP-VL sequence encoded by the plasmid deposited at the ATCC and having ATCC Accession No. PTA0-127199; an IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

In some aspects, the disclosure provides anti-IL-4/IL-13/TSLP antibodies comprising the sequence encoded by the plasmid deposited at the ATCC and having ATCC Accession No. PTA-127202; the sequence encoded by the plasmid deposited at the ATCC and having ATCC Accession No. PTA-127192; the sequence encoded by the plasmid deposited at the ATCC and having ATCC Accession No. PTA-127201; the sequence encoded by the plasmid deposited at the ATCC and having ATCC Accession No. PTA-127194; and the sequence encoded by the plasmid deposited at the ATCC and having ATCC Accession No. PTA-127193.

### Antibodies to IL-4/IL-13/p40

IL-4/IL-13/p40 antibodies of the disclosure, (for example, IL134P40-0705) combine a p40 binding domain with the binding domains for IL-4 and IL-13. p40 (also known as IL-12p40, also known as IL12B), is a subunit of the heterodimeric cytokines IL-12 and IL-23 (75). As a component of IL-12, p40 is disulfide-bonded to IL-12p35 (IL12A); as a component of IL-23, it is disulfide-bonded to IL-23p19 (IL23A). IL-12 and IL-23 signal through different heterodimeric receptor complexes, each of with contain a common IL-12Rb1 subunit that binds p40 and a cytokine-specific subunit that confers selectivity for IL-12 or IL-23 (IL-12Rb2 or IL-23R, respectively). IL-12 and IL-23 are produced primarily by innate immune cells, such as monocytes, macrophages, dendritic cells, and neutrophils, and are key cytokines acting at the interface of innate and adaptive immunity (76).

IL-4 and IL-13 are linked primarily to type 2 effector responses. In contrast, IL-12 and IL-23 are implicated in type 1 and type 3 (Th17) responses, respectively (77). IL-12 drives T helper 1 (Th1) cell differentiation and interferon-y (IFN-γ) production, whereas IL-23 promotes the maintenance of Th17 cells that produce IL-17 and other type 3 cytokines. Type 1 and type 3 responses have been implicated in a range of human inflammatory and autoimmune diseases. A causal role for p40-containing cytokines has been established through numerous drug approvals (75). The p40 neutralizing agent Stelara® (ustekinumab; Jannsen) neutralizes both IL-12 and IL-23, and is approved for the treatment of plaque psoriasis, psoriatic arthritis, Crohn's disease, and ulcerative colitis.

Human diseases cannot always be categorized as strictly type 1, type 2, or type 3. For example, while AD is a type 2-driven disease, certain subsets of AD patients also have elevated type 1 and type 3 signatures (78). Therefore, although type 2 responses are clearly causal in AD, and p40 suppression alone is not superior to topical corticosteroids in AD trials (79, 80), combining p40 inhibition with IL-4 and IL-13 inhibition could suppress a component of disease that is unaddressed by type 2 suppression alone to deliver transformational efficacy in AD patients, or in AD patient subsets (81). Other human diseases provide more prominent examples of a mixed inflammatory signature. Certain asthma subtypes are characterized by eosinophilic and neutrophilic cellular infiltrates, indicating both type 2 and type 1/3 involvement (82). Systemic sclerosis has an underlying IL-4/13 component as well as an underlying Th17 component (83, 84, 85), and human genetic studies have identified mutations which result in reduced expression of the IL-12 and IL-23 common receptor subunit IL-12Rb1 as being protective alleles (86). In addition, liver biopsies from patients with non-alcoholic steatohepatitis (NASH) patients have clear signatures of type 1, type 2, and type 3 inflammation (87). And as a final example, alopecia areata has a mixed signature (88), and there are positive data in alopecia patients following treatment with either Dupixent® or Stelara® (89, 90).

These data support the hypothesis that concomitant blockade of IL-4, IL-13, p40 will be efficacious in type 2-driven human diseases with underlying type 1 and type 3 signatures, and also in disease with complex etiology that cannot be simply be categorized as type 1, type 2, or type 3. Another potential benefit of concomitant blockade is that type 1, type 2, and type 3 responses negatively regulate one another. For example, pre-clinical studies have suggested that type 2 and type 3 responses are reciprocally regulated, and that pharmacological inhibition of one can result in a “rebound” response in the other (91, 92). Data are now emerging that such reciprocal regulation could also occur in humans, as there are now examples of AD patients taking Dupilumab® who develop diseases classically associated with Th1 and Th17 mediated inflammation such as psoriasis, inflammatory arthritis, and enthesitis (93, 94). The side effect of conjunctivitis associated with Dupixent® use has also been attributed to an elevation of Th1 and Th17 cellular infiltrates in the eyes of type 2-suppressed individuals (95). Therefore, combined blockade of IL-4, IL-13, and p40 with IL-4/IL-13/p40 antibodies of the disclosure, (for example, IL134P40-0705) would address not only the underlying signatures present in the disease state, but potentially also any unwanted rebound responses that may result from therapeutic intervention with type 1, type 2, or type 3 blocking agents in isolation.

The disclosure provides antibodies that bind to IL-4, IL-13, and p40. As used herein, the terms IL-4, IL-13, and p40 include variants, isoforms, homologs, orthologs and paralogs of one or more of IL-4, IL-13, and p40 respectively. In some embodiments, an antibody disclosed herein cross-reacts with one or more of IL-4, IL-13, and p40 from species other than human, such as IL-4, IL-13, and p40 of cynomolgus monkey. In some embodiments, an antibody may be completely specific for human IL-4, IL-13, and p40 and may not exhibit species cross-reactivity or other types of cross-reactivity. As used herein the term IL-4, IL-13, and p40 refers to naturally occurring human IL-4, IL-13, and p40 unless contextually dictated otherwise. An “IL-4/IL-13/p40 antibody” “anti-IL-4/IL-13/p40 antibody” or other similar designation means any antibody (as defined herein) that binds or reacts with IL-4, IL-13, and p40, an isoform, fragment or derivative thereof.

In some embodiments, the invention provides an IL-4/IL-13/p40 antibody having a light chain variable region (VL) sequence and a heavy chain variable region (VH) sequence as found in Table 86 or variants thereof.

The invention also provides CDR portions of IL-4/IL-13/p40 antibodies. Determination of CDR regions is defined in Example 1. In some embodiments, the IL-4/IL-13/p40 antibody comprises the six CDRs of an IL-4 antibody selected from Table 80 or 86, the six CDRs of an IL-13 antibody selected from Table 81 or 86, and the six CDRs of a p40 antibody selected from Table 86. In some embodiments, the antibody comprises the VH and VL of an IL-4 antibody selected from Table 80 or Table 86, the VH and VL of an IL-13 antibody selected from Table 81 or Table 86, and the VH and VL of a p40 antibody selected from Table 86. In some embodiments, the IL-4/IL-13/p40 antibody comprises sequences selected from Table 86.

In some embodiments, the disclosure provides anti-IL-4/IL-13/p40 antibodies containing variations of the CDRs, VH, VL, HC, and LC regions shown in one or more of Tables 80, 81, 86, and 87, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in in one or more of Tables 80, 81, 86, and 87. These amounts are not meant to be limiting and increments between the recited percentages are specifically envisioned as part of the disclosure.

The present disclosure relates to an isolated antibody that specifically binds to p40, specifically binds to IL-4, and specifically binds to IL-13, comprising a p40 binding domain, an IL-4 binding domain, and an IL-13 binding domain. The IL-4/IL-13/p40 antibody may comprise
(i) a p40 binding domain comprising a heavy chain variable region (p40-VH) and a light chain variable region (p40-VL), wherein the CDR-H1 of the p40 binding domain comprises the amino acid sequence of SEQ ID NO: 166; the CDR-H2 of the p40 binding domain comprises the amino acid sequence of SEQ ID NO: 167; the CDR-H3 of the p40 binding domain comprises the amino acid sequence of SEQ ID NO: 168; the CDR-L1 of the p40 binding domain comprises the amino acid sequence of SEQ ID NO: 171; the CDR-L2 of the p40 binding domain comprises the amino acid sequence of SEQ ID NO: 172, and the CDR-L3 of the p40 binding domain comprises the amino acid sequence of SEQ ID NO: 173; and
(ii) a IL-4 binding domain comprising a heavy chain variable region (IL4-VH) and a light chain variable region (IL4-VL), wherein the CDR-H1 of the IL-4 binding domain comprises the amino acid sequence of SEQ ID NO: 18; the CDR-H2 of the IL-4 binding domain comprises the amino acid sequence of SEQ ID NO: 2; the CDR-H3 of the IL-4 binding domain comprises the amino acid sequence of SEQ ID NO: 3; the CDR-L1 of the IL-4 binding domain comprises the amino acid sequence of SEQ ID NO: 24; the CDR-L2 of the IL-4 binding domain comprises the amino acid sequence of SEQ ID NO: 12, and the CDR-L3 of the IL-4 binding domain comprises the amino acid sequence of SEQ ID NO: 25, and
(iii) a IL-13 binding domain comprising a heavy chain variable region (IL13-VH) and a light chain variable region (IL13-VL), wherein the CDR-H1 of the IL-13 binding domain comprises the amino acid sequence of SEQ ID NO: 41; the CDR-H2 of the IL-13 binding domain comprises the amino acid sequence of SEQ ID NO: 42; the CDR-H3 of the IL-13 binding domain comprises the amino acid sequence of SEQ ID NO:-50; the CDR-L1 of the IL-13 binding domain comprises the amino acid sequence of SEQ ID NO: 53; the CDR-L2 of the IL-13 binding domain comprises the amino acid sequence of SEQ ID NO: 37, and the CDR-L3 of the IL-13 binding domain comprises the amino acid sequence of SEQ ID NO: 38.
   In some aspects of the disclosure, the IL-4/IL-13/TSLP antibody may comprise
(i) a p40 binding domain comprising p40-VH of SEQ ID NO: 169, and a p40-VL of SEQ ID NO: 175;
(ii) an IL-4 binding domain comprising a IL4-VH of SEQ ID NO: 22, and a IL4-VL of SEQ ID NO: 26;
(iv) an IL-13 binding domain comprising a IL13-VH of SEQ ID NO: 51, and a IL13-VL of SEQ ID NO: 54.

The p40 binding domain may be fused to the IL-4 binding domain. The p40 binding domain may be fused to the IL-13 binding domain. The IL-13 binding domain may be fused to the IL-4 binding domain. The fusion may be with or without a linker. If present, the linker may comprise a sequence of amino acids between 2 and 20 amino acids in length. The linker may consist of one or more amino acids selected from glycine, alanine, and serine. The linker may consist of one or more amino acids selected from glycine and serine. The linker may comprise SEQ ID NO: 104.

IL-4/IL-13/p40 antibodies of the invention may comprise a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site.

In some aspects, the first antigen binding site specifically binds IL-4, the second antibody binding site specifically binds IL-13, and the third antigen binding site specifically binds p40. In some aspects, the first antigen binding site specifically binds IL-4, the second antibody binding site specifically binds p40, and the third antigen binding site specifically binds IL-13. In some aspects, the first antigen binding site specifically binds IL-13, the second antibody binding site specifically binds IL-4, and the third antigen binding site specifically binds p40. In some aspects, the first antigen binding site specifically binds IL-13, the second antibody binding site specifically binds p40, and the third antigen binding site specifically binds IL-4. In some aspects, the first antigen binding site specifically binds p40, the second antibody binding site specifically binds IL-13, and the third antigen binding site specifically binds IL-4. In some aspects, the first antigen binding site specifically binds p40, the second antibody binding site specifically binds IL-4, and the third antigen binding site specifically binds IL-13.

The IL-4/IL-13/p40 antibody may comprise one or more domain-swap Fab domains. The domain-swap Fab domains may be selected from the group consisting of p40-xFab, IL4-xFab, and IL13-xFab.

The IL-4/IL-13/p40 antibody may comprise a first Fab domain comprising the IL13-Fab, a second Fab domain comprising IL4-Fab, and a third Fab domain comprising the p40-Fab. To facilitate formation of the multispecific antibody, the first polypeptide may comprises a first Fc chain and the second polypeptide may comprises a second Fc chain. The first Fc chain and the second Fc chain may each comprise one or more amino acid modifications that promote the association of the first Fc chain with the second Fc chain.

In some aspects, the first Fc chain comprises a first CH3 domain, and the second Fc chain comprises a second CH3 domain, and the first CH3 domain and the second CH3 domain each comprise a different and complementary sequence, and the different and complementary sequences are selected from one of the following pairs of different and complementary sequences: (i) SEQ ID NO: 106 and SEQ ID NO:111; (ii) SEQ ID NO:147 and SEQ ID NO: 148; and (iii) SEQ ID NO: 124, and SEQ ID NO:127.

The disclosure provides an IL-4/IL-13/p40 antibody, wherein the identity of the first, second, third, fourth, and fifth polypeptide chains is selected from the group consisting of
(i) the first polypeptide chain comprises SEQ ID NO: 146, the second polypeptide chain comprises SEQ ID NO: 178, the third polypeptide chain comprises SEQ ID NO: 109, the fourth polypeptide chain comprises SEQ ID NO: 196, and the fifth polypeptide chain comprises SEQ ID NO: 177;
(ii) the first polypeptide chain comprises SEQ ID NO: 112, the second polypeptide chain comprises SEQ ID NO: 179, the third polypeptide chain comprises SEQ ID NO: 196, the fourth polypeptide chain comprises SEQ ID NO: 109, and the fifth polypeptide chain comprises SEQ ID NO: 177;
(iii) the first polypeptide chain comprises SEQ ID NO: 181, the second polypeptide chain comprises SEQ ID NO 180, the third polypeptide chain comprises SEQ ID NO: 182, the fourth polypeptide chain comprises SEQ ID NO: 109, and the fifth polypeptide chain comprises SEQ ID NO: 122;
(iv) the first polypeptide chain comprises SEQ ID NO: 118, the second polypeptide chain comprises SEQ ID NO: 183, the third polypeptide chain comprises SEQ ID NO: 120, the fourth polypeptide chain comprises SEQ ID NO: 116, and the fifth polypeptide chain comprises SEQ ID NO: 177;
(v) the first polypeptide chain comprises SEQ ID NO: 185, the second polypeptide chain comprises SEQ ID NO: 125, the third polypeptide chain comprises SEQ ID NO: 176, the fourth polypeptide chain comprises SEQ ID NO: 207, and the fifth polypeptide chain comprises SEQ ID NO: 122;
(vi) the first polypeptide chain comprises SEQ ID NO: 185, the second polypeptide chain comprises SEQ ID NO: 125, the third polypeptide chain comprises SEQ ID NO: 176, the fourth polypeptide chain comprises SEQ ID NO: 27, and the fifth polypeptide chain comprises SEQ ID NO: 122; and
(vii) the first polypeptide chain comprises SEQ ID NO: 186, the second polypeptide chain comprises SEQ ID NO: 130, the third polypeptide chain comprises SEQ ID NO: 176, the fourth polypeptide chain comprises SEQ ID NO: 27, and the fifth polypeptide chain comprises SEQ ID NO: 122.

In some aspects, the disclosure provided an isolated antibody that specifically binds p40, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a p40 binding site, and wherein
the first polypeptide chain comprises SEQ ID NO: 186, the second polypeptide chain comprises SEQ ID NO: 130, the third polypeptide chain comprises SEQ ID NO: 176, the fourth polypeptide chain comprises SEQ ID NO: 27, and the fifth polypeptide chain comprises SEQ ID NO: 122.

The anti-IL-4/IL-13p40 antibodies of the present disclosure demonstrate a combination of improved anti-IL-4 and anti-IL-13 activity over parental antibodies while minimizing an increase in viscosity.

Anti-IL-4/IL-13/p40 antibodies of the present disclosure may have a viscosity of less than 20 cP at concentrations of at least 100 mg/mL in a buffer of 20 mM Histidine, 8.5% sucrose, 0.05 mg/mL EDTA pH 6.0. Anti-IL-4/IL-13/p40 antibodies of the present disclosure may have a viscosity of less than 20 cP at concentrations of at least 110 mg/mL in a buffer of 20 mM Histidine, 8.5% sucrose, 0.05 mg/mL EDTA pH 6.0. Anti-IL-4/IL-13/p40 antibodies of the present disclosure may have a viscosity of less than 16 cP at concentrations of at least 100 mg/mL in a buffer of 20 mM Histidine, 8.5% sucrose, 0.05 mg/mL EDTA pH 6.0. Anti-IL-4/IL-13/p40 antibodies of the present disclosure may have a viscosity of less than 16 cP at concentrations of at least 110 mg/mL in a buffer of 20 mM Histidine, 8.5% sucrose, 0.05 mg/mL EDTA pH 6.0. Anti-IL-4/IL-13/p40 antibodies of the present disclosure may have a viscosity of less than 12 cP at concentrations of at least 50 mg/mL in a buffer of 20 mM Histidine, 8.5% sucrose, 0.05 mg/mL EDTA pH 6.0. Anti-IL-4/IL-13/p40 antibodies of the present disclosure may have a viscosity of less than 12 cP at concentrations of at least 70 mg/mL in a buffer of 20 mM Histidine, 8.5% sucrose, 0.05 mg/mL EDTA pH 6.0. Anti-IL-4/IL-13/p40 antibodies of the present disclosure may have a viscosity of less than 12 cP at concentrations of at least 80 mg/mL in a buffer of 20 mM Histidine, 8.5% sucrose, 0.05 mg/mL EDTA pH 6.0. Anti-IL-4/IL-13/p40 antibodies of the present disclosure may have a viscosity of less than 12 cP at concentrations of at least 90 mg/mL in a buffer of 20 mM Histidine, 8.5% sucrose, 0.05 mg/mL EDTA pH 6.0. Anti-IL-4/IL-13/p40 antibodies of the present disclosure may have a viscosity of less than 11 cP at concentrations of at least 94 mg/mL in a buffer of 20 mM Histidine, 8.5% sucrose, 0.05 mg/mL EDTA pH 6.0.

Anti-IL-4/IL-13/p40 antibodies of the present disclosure may have a terminal half-life of at least 12 days in cynomolgus monkeys. Anti-IL-4/IL-13/p40 antibodies of the present disclosure may have a terminal half-life of at least 18 days in TG32 mice.

Anti-IL-4/IL-13/p40 antibodies of the disclosure may bind to human IL-4 with a binding affinity of less than 220 nM, as measured by SPR. Favorably, Anti-IL-4/IL-13/p40 antibodies do not specifically bind to mouse, rat, or rabbit IL-4.

Anti-IL-4/IL-13/p40 antibodies of the disclosure may bind to human IL-4 with a binding affinity of less than 1 pM, as measured by KinExA in a fixed antigen assay in PBS. Anti-IL-4/IL-13/p40 antibodies of the present disclosure may be characterized by an affinity constant of less than 0.8 pM for human IL-4, as measured by kinetics exclusion assay in a fixed antigen assay in PBS. Anti-IL-4/IL-13/p40 antibodies of the present disclosure may be characterized by an affinity constant of less than 1 pM for cynomolgus IL-4, as measured by kinetics exclusion assay in a fixed antigen assay in PBS. Anti-IL-4/IL-13/p40 antibodies of the present disclosure may be characterized by an affinity constant of between 0.7-0.8 pM for human IL-4, as measured by kinetics exclusion assay in a fixed antigen assay in PBS.

Anti-IL-4/IL-13/p40 antibodies of the present disclosure may be characterized by an affinity constant of less than 2 pM for human IL-13, as measured by kinetics exclusion assay. Anti-IL-4/IL-13/p40 antibodies of the present disclosure may be characterized by an affinity constant of les than 1.6 pM for human IL-13, as measured by kinetics exclusion assay in PBS. Anti-IL-4/IL-13/p40 antibodies of the present disclosure may be characterized by an affinity constant of less than 0.5 pM for cynomolgus IL-13, as measured by kinetics exclusion assay in PBS.

Anti-IL-4/IL-13/p40 antibodies of the disclosure may bind to human IL-13 with a binding affinity of less than 220 nM, as measured by SPR. Favorably, Anti-IL-4/IL-13/p40 antibodies do not specifically bind to mouse, rat, or rabbit IL-13.

Anti-IL-4/IL-13/p40 anti bodies of the present disclosure may be characterized by binding affinity constant of less than 130 pM for human IL-12, as measured by surface plasmon resonance. Anti-IL-4/IL-13/p40 antibodies of the present disclosure may be characterized by an affinity constant of between 100-130 pM for human IL-12, as measured by surface plasmon resonance. Anti-IL-4/IL-13/p40 antibodies of the present disclosure may be characterized by an affinity constant of between 110-120 pM for human IL-12, as measured by surface plasmon resonance. Anti-IL-4/IL-13/p40 anti bodies of the present disclosure may be characterized by binding affinity constant of less than 260 pM for cynomolgus IL-12, as measured by surface plasmon resonance. Anti-IL-4/IL-13/p40 antibodies of the present disclosure may be characterized by an affinity constant of less than 100 pM for human IL-23, as measured by surface plasmon resonance. Anti-IL-4/IL-13/p40 antibodies of the present disclosure may be characterized by an affinity constant of between 80-100 pM for human IL-23, as measured by surface plasmon resonance. Anti-IL-4/IL-13/p40 antibodies of the present disclosure may be characterized by an affinity constant of between 85-95 pM for human IL-23, as measured by surface plasmon resonance. Anti-IL-4/IL-13/p40 antibodies of the present disclosure may be characterized by an affinity constant of less than 250 pM for cynomolgus IL-23, as measured by surface plasmon resonance.

Anti-IL-4/IL-13/p40 antibodies of the present disclosure may be characterized by an IC₅₀ of less than 12 pM in a human monocyte assay for neutralization of IL-4 induction of CD23. Anti-IL-4/IL-13/p40 antibodies of the present disclosure may be characterized by an IC₅₀ of less than 12 pM in a human monocyte assay for neutralization of cynomolgus monkey IL-4 induction of CD23. Anti-IL-4/IL-13/p40 antibodies of the present disclosure may be characterized by an IC₅₀ of less than 25 pM in a human monocyte assay for neutralization of IL-4 induction of CD23. Anti-IL-4/IL-13/p40 antibodies of the present disclosure may be characterized by an IC₅₀ of less than 20 pM in a human monocyte assay for neutralization of IL-4 induction of CD23. IL-4 neutralization may be determined by flow cytometry determination of CD23 positive cells.

Anti-IL-4/IL-13/p40 antibodies of the present disclosure may be characterized by an IC₅₀ of less than 12 pM in a human monocyte assay for neutralization of IL-13 induction of CD23. Anti-IL-4/IL-13/p40 antibodies of the present disclosure may be characterized by an IC₅₀ of less than 45 pM in a human monocyte assay for neutralization of cynomolgus monkey IL-13 induction of CD23. Anti-IL-4/IL-13/p40 antibodies of the present disclosure may be characterized by an IC₅₀ of less than 12 pM in a human monocyte assay for neutralization of IL-13 induction of CD23. Anti-IL-4/IL-13/p40 antibodies of the present disclosure may be characterized by an IC₅₀ of less than 50 pM in a human monocyte assay for neutralization of cynomolgus monkey IL-13 induction of CD23. IL-13 neutralization may be determined by flow cytometry determination of CD23 positive cells.

Anti-IL-4/IL-13/p40 antibodies of the present disclosure may be characterized by an IC₅₀ of less than 600 pM in a human IL-12 neutralization Kit-225 assay. Anti-IL-4/IL-13/p40 antibodies of the present disclosure may be characterized by an IC₅₀ of less than 2100 pM in a human IL-23 neutralization Kit-225 assay. Anti-IL-4/IL-13/p40 antibodies of the present disclosure may be characterized by an IC₅₀ of less than 400 pM in a cynomolgus IL-12 neutralization Kit-225 assay. Anti-IL-4/IL-13/p40 antibodies of the present disclosure may be characterized by an IC₅₀ of less than 3100 pM in a cynomolgus IL-23 neutralization Kit-225 assay. The Kit-225 assay is a flow cytometry assessment for STAT4 or STAT3 to determine the ability of the antibody to prevent IL-12 induced STAT4 phosphorylation or the ability of the antibody to prevent IL-23 induced STAT3 phosphorylation respectively in the KIT-225 cell line.

Anti-IL-4/IL-13/p40 antibodies of the present disclosure may be characterized by an IC₅₀ of less than 400 pM in a human IL-12 neutralization whole blood assay. Anti-IL-4/IL-13/p40 antibodies of the present disclosure may be characterized by an IC₅₀ of less than 10,000 pM in a human IL-23 neutralization whole blood assay. The whole blood assay is a flow cytometry assessment for STAT4 or STAT3 to determine the ability of the antibody to prevent IL-12 induced STAT4 phosphorylation or the ability of the antibody to prevent IL-23 induced STAT3 phosphorylation respectively in human whole blood cells.

Anti-IL-4/IL-13/p40 antibodies of the present disclosure may be characterized by an IC₅₀ of between 140-170 pM for human IL-12, as measured by a Kit-225 assay. Anti-IL-4/IL-13/p40 antibodies of the present disclosure may be characterized by an IC₅₀ of between 150-160 pM for human IL-12, as measured by a Kit-225 assay. Anti-IL-4/IL-13/p40 antibodies of the present disclosure may be characterized by an IC₅₀ of between 800-900 pM for human IL-23, as measured by a Kit-225 assay. Anti-IL-4/IL-13/p40 antibodies of the present disclosure may be characterized by an IC₅₀ of between 840-860 pM for human IL-23, as measured by a Kit-225 assay. The Kit-225 assay is a flow cytometry assessment for STAT4 or STAT3 to determine the ability of the antibody to prevent IL-12 induced STAT4 phosphorylation or the ability of the antibody to prevent IL-23 induced STAT3 phosphorylation respectively in the KIT-225 cell line.

Anti-IL-4/IL-13/p40 antibodies of the present disclosure may be characterized by an IC₅₀ of less between 210-300 pM in an IL-12 neutralization whole blood assay. Anti-IL-4/IL-13/p40 antibodies of the present disclosure may be characterized by an IC₅₀ of between 250-270 pM in an IL-12 neutralization whole blood assay. Anti-IL-4/IL-13/p40 antibodies of the present disclosure may be characterized by an IC₅₀ of less between 4000-5000 pM in an IL-23 neutralization whole blood assay. Anti-IL-4/IL-13/p40 antibodies of the present disclosure may be characterized by an IC₅₀ of between 4500-4520 pM in an IL-23 neutralization whole blood assay. The whole blood assay is a flow cytometry assessment for STAT4 or STAT3 to determine the ability of the antibody to prevent IL-12 induced STAT4 phosphorylation or the ability of the antibody to prevent IL-23 induced STAT3 phosphorylation respectively in human whole blood cells.

In some aspects, the disclosure provides an isolated anti-IL-4/IL-13/p40 antibody that specifically bind p40 through an p40 heavy chain variable region (p40-VH) and an p40 light chain variable region (p40-VL); that specifically binds IL-4 through an IL-4 heavy chain variable region (IL4-VH) and an IL-4 light chain variable region (IL4-VL); and that specifically binds IL-13 through an IL-13 heavy chain variable region (IL13-VH) and an IL-13 light chain variable region (IL33-VL); wherein the p40-VH comprises the CDR-H1, CDR-H2, and CDR-H3 sequences encoded by the plasmid deposited at the ATCC and having ATCC Accession No. PTA-127206, and the p40-VL comprises the CDR-L1, CDR-L2, and CDR-L3 sequences encoded by the plasmid deposited at the ATCC and having ATCC Accession No. PTA-127205, and the IL4-VH comprises the CDR-H1, CDR-H2, and CDR-H3 sequences encoded by the plasmid deposited at the ATCC and having ATCC Accession No. PTA-127198, and the IL4-VL comprises the CDR-L1, CDR-L2, and CDR-L3 sequences encoded by the plasmid deposited at the ATCC and having ATCC Accession No. PTA-127197, and the IL13-VH comprises the CDR-H1, CDR-H2, and CDR-H3 sequences encoded by the plasmid deposited at the ATCC and having ATCC Accession No. PTA-127196, and the IL13-VL comprises the CDR-L1, CDR-L2, and CDR-L3 sequences encoded by the plasmid deposited at the ATCC and having ATCC Accession No. PTA-127195.

In some aspects the disclosure provides anti-IL-4/IL-13/p40 antibodies comprising a p40-VH sequence encoded by the plasmid deposited at the ATCC and having ATCC Accession No. PTA-127206; a p40-VL sequence encoded by the plasmid deposited at the ATCC and having ATCC Accession No. PTA-127205; an IL4-VH sequence encoded by the plasmid deposited at the ATCC and having ATCC Accession No. PTA-127198; an IL4-VL sequence encoded by the plasmid deposited at the ATCC and having ATCC Accession No. PTA-127197; an IL13-VH sequence encoded by the plasmid deposited at the ATCC and having ATCC Accession No. PTA-127196; and an IL13-VL sequence encoded by the plasmid deposited at the ATCC and having ATCC Accession No. PTA-127195.

In some aspects the disclosure provides anti-IL-4/IL-13/p40 antibodies comprising the sequence encoded by the plasmid deposited at the ATCC and having ATCC Accession No. PTA-127204; the sequence encoded by the plasmid deposited at the ATCC and having ATCC Accession No. PTA-127192; the sequence encoded by the plasmid deposited at the ATCC and having ATCC Accession No. PTA-127203; the sequence encoded by the plasmid deposited at the ATCC and having ATCC Accession No. PTA-127194; and the sequence encoded by the plasmid deposited at the ATCC and having ATCC Accession No. PTA-127193.

### Anti-PD-1 Antibodies and Oncology Applications

In some aspects of the disclosure, one or more of IL-4, IL-13, TSLP, IL-13, IL-4/IL-13, IL-4/IL-13/TSLP, IL-4/IL-13/IL-33, and IL-4/IL-13/p40 antibodies of the disclosure may be combined with a PD-1 pathway antagonist. In some aspects of the disclosure, one or more of IL-4, IL-13, TSLP, IL-13, IL-4/IL-13, IL-4/IL-13/TSLP, and IL-4/IL-13/IL-33, antibodies of the disclosure may be combined with a PD-1 pathway antagonist. In some aspects of the disclosure, one or more of IL-4, IL-13, TSLP, IL-13, IL-4/IL-13, and IL-4/IL-13/TSLP. antibodies of the disclosure may be combined with a PD-1 pathway antagonist.

In some aspects of the disclosure IL-4/IL-13/TSLP antibodies of the disclosure may be combined with a PD-1 pathway antagonist. This provides significant advantages of combining multiple orthogonal pathways that can impact discrete oncological targets that may not be fully addressed by, for example, an IL-4/IL-13 combination with a PD-1 pathway antagonist, or by IL-4/IL13/IL-33, with a PD-1 antagonist, where the IL-33 antagonism may be redundant without providing the additional advantages that antagonizing the TSLP pathway provides. The disclosure therefore provides uses of IL-4 antibodies, IL-13 antibodies, TSLP antibodies, and PD-1 pathway antagonists for the treatment of cancers, tumor reduction, and the like. Further, the disclosure provides uses of IL-4/IL-13/TSLP antibodies, and PD-1 pathway antagonists for the treatment of cancers, tumor reduction, and the like. Preferably, the PD-1 antagonist is administered as a separate molecule to each of the IL-4, IL-13, and TSLP antibodies.

Programmed cell death protein 1, also known as PD-1 and CD279 (cluster of differentiation 279), is a protein on the surface of T and B cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells. The PD-1 protein in humans is encoded by the PDCD1 gene. PD-1 is a cell surface receptor that belongs to the immunoglobulin superfamily and is expressed on T cells and pro-B cells. PD-1 binds two ligands, PD-L1 and PD-L2.

PD-1 is an immune checkpoint and guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells).

PD-L1, the ligand for PD1, is highly expressed in several cancers. Many tumor cells express PD-L1, an immunosuppressive PD-1 ligand; inhibition of the interaction between PD-1 and PD-L1 can enhance T-cell responses in vitro and mediate preclinical antitumor activity. This is known as immune checkpoint blockade.

In some aspects of the disclosure, one or more of IL-4, IL-13, TSLP, IL-13, IL-4/IL-13, IL-4/IL-13/TSLP, IL-4/IL-13/IL-33, and IL-4/IL-13/p40 antibodies of the disclosure may be combined with a PD-1 pathway antagonist. The PD-1 pathway antagonist may be an anti-PD-1 antagonist antibody or anti-PD-L1 antibody. The programmed death 1 (PD-1) receptor and PD-1 ligands 1 and 2 (PD-L1 and PD-L2, respectively) play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression (Dong et al., Nat Med 1999; 5:1365-69; Freeman et al. J Exp Med 2000; 192:1027-34), potentially providing an immune-tolerant environment for tumor development and growth. Conversely, inhibition of this interaction can enhance local T-cell responses and mediate antitumor activity in nonclinical animal models (Iwai Y, et al. Proc Natl Acad Sci USA 2002; 99:12293-97).

Several anti-PD-1 pathway therapeutics have been approved, including, Pidilizumab (CT-011, Cure Tech), BMS-936559 (Bristol Myers Squibb) and Toripalimab (JS-001, TopAlliance). Both Atezolizumab (MPDL3280A, Roche) Avelumab (Merck KGaA, Darmstadt, Germany & Pfizer) target the similar PD-L1 receptor.

Examples of anti-PD-1 antibodies that are useful in the treatment method, medicaments and uses of the present invention include BCD-100, camrelizumab, cemiplimab, genolimzumab (CBT-501), MEDI0680, nivolumab, pembrolizumab, sintilimab, spartalizumab, STI-A1110, tislelizumab, atezolizumab, durvalumab, BMS-936559 (MDX-1105), LY3300054, and TSR-042. In some embodiments, the anti-PD-1 antibody has a VH as shown in SEQ ID NO: 4 and a VL as shown in SEQ ID NO: 8 of U.S. Ser. No. 10/155,037. In some embodiments, the anti-PD-1 antibody is sasanlimab, (PF-06801591 (RN888), a humanized IgG4 monoclonal antagonist antibody, Pfizer) (see WO2016/092419).

PD-1 pathway antagonist antibodies may have any suitable format. For example, therapeutic antibodies may have any format as described elsewhere herein. The PD-1 pathway antagonist antibody may be a naked antibody. The PD-1 pathway antagonist antibody may be linked to a drug/agent (also known as an “antibody-drug conjugate” (ADC)). In some embodiments, the PD-1 pathway antagonist antibody against a particular antigen may incorporated into a multi-specific antibody (e.g. a bispecific antibody).

In some embodiments, an antibody directed to an antigen may be conjugated to a drug/agent. Linked antibody-drug molecules are also referred to as “antibody-drug conjugates” (ADCs). Drugs/agents can be linked to an antibody either directly or indirectly via a linker Most commonly, toxic drugs are linked to an antibody, such that binding of the ADC to the respective antigen promotes the killing of cells that express the antigen. For example, ADCs that are linked to toxic drugs are particularly useful for targeting tumor associated antigens, in order to promote the killing of tumor cells that express the tumor associated antigens. In other embodiments, agents that may be linked to an antibody may be, for example, an immunomodulating agent (e.g. to modulate the activity of immune cells in the vicinity of the ADC), an imaging agent (e.g. to facilitate the imaging of the ADC in a subject or a biological sample from the subject), or an agent to increase the antibody serum half-life or bioactivity.

Methods for conjugating cytotoxic agent or other therapeutic agents to antibodies have been described in various publications. For example, chemical modification can be made in the antibodies either through lysine side chain amines or through cysteine sulfhydryl groups activated by reducing interchain disulfide bonds for the conjugation reaction to occur. See, e.g., Tanaka et al., FEBS Letters 579:2092-2096, 2005, and Gentle et al., Bioconjugate Chem. 15:658-663, 2004. Reactive cysteine residues engineered at specific sites of antibodies for specific drug conjugation with defined stoichiometry have also been described. See, e.g., Junutula et al., Nature Biotechnology, 26:925-932, 2008. Conjugation using an acyl donor glutamine-containing tag or an endogenous glutamine made reactive (i.e., the ability to form a covalent bond as an acyl donor) by polypeptide engineering in the presence of transglutaminase and an amine (e.g., a cytotoxic agent comprising or attached to a reactive amine) is also described in international applications WO2012/059882 and WO2015015448. In some embodiments, an ADC may have any of the features or characteristics of the ADCs provided in WO2016166629, which is hereby incorporated by reference for all purposes.

Drugs or agents that can be linked to an antibody in the ADC format can include, for example, cytotoxic agents, immunomodulating agents, imaging agents, therapeutic proteins, biopolymers, or oligonucleotides.

Exemplary cytotoxic agents that may be incorporated in an ADC include an anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, a pladienolide, and stereoisomers, isosteres, analogs, or derivatives thereof. Exemplary immunomodulating agents that may be incorporated in an ADC include gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, cytokines, stem cell growth factors, lymphotoxins, tumor necrosis factor (TNF), hematopoietic factors, interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-.alpha., -.beta. and -.gamma.), the stem cell growth factor designated “S 1 factor,” erythropoietin and thrombopoietin, or a combination thereof.

Exemplary imaging agents that may be included in an ADC include fluorescein, rhodamine, lanthanide phosphors, and their derivatives thereof, or a radioisotope bound to a chelator. Examples of fluorophores include, but are not limited to, fluorescein isothiocyanate (FITC) (e.g., 5-FITC), fluorescein amidite (FAM) (e.g., 5-FAM), eosin, carboxyfluorescein, erythrosine, Alexa Fluor® (e.g., Alexa 350, 405, 430, 488, 500, 514, 532, 546, 555, 568, 594, 610, 633, 647, 660, 680, 700, or 750), carboxytetramethylrhodamine (TAMRA) (e.g., 5,-TAMRA), tetramethylrhodamine (TMR), and sulforhodamine (SR) (e.g., SR101). Examples of chelators include, but are not limited to, 1,4,7,10-tetraazacyclododecane-N,N′,N″,N′″-tetraacetic acid (DOTA), 1,4,7-triazacyclononane-1,4,7-triacetic acid (NOTA), 1,4,7-triazacyclononane, 1-glutaric acid-4,7-acetic acid (deferoxamine), diethylenetriaminepentaacetic acid (DTPA), and 1,2-bis(o-aminophenoxy)ethane-N,N,N′,N′-tetraacetic acid) (BAPTA).

Exemplary therapeutic proteins that may be included in an ADC include a toxin, a hormone, an enzyme, and a growth factor.

Exemplary biocompatible polymers that may be incorporated in an ADC include water-soluble polymers, such as polyethylene glycol (PEG) or its derivatives thereof and zwitterion-containing biocompatible polymers (e.g., a phosphorylcholine containing polymer).

Exemplary biocompatible polymers that may be incorporated in an ADC include anti-sense oligonucleotides.

In some embodiments, the PD-1 pathway antagonist antibody antagonizes PD-L1. Examples of mAbs that bind to human PD-L1 include antibodies described in WO2013079174, WO2015061668, WO2010089411, WO/2007/005874, WO/2010/036959, WO/2014/100079, WO2013/019906, WO/2010/077634, and U.S. Pat. Nos. 8,552,154, 8,779,108, and 8,383,796.

A combination therapy provided herein may comprise one or more chemotherapeutic agents. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1l and calicheamicin phil1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, fluridil, apalutamide, enzalutamide, cimetidine and goserelin; KRAS inhibitors; MCT4 inhibitors; MAT2a inhibitors; tyrosine kinase/vascular endothelial growth factor (VEGF) receptor inhibitors such as sunitinib, axitinib, sorafenib, tivozanib; alk/c-Met/ROS inhibitors such as crizotinib, lorlatinib; mTOR inhibitors such as temsirolimus, gedatolisib; src/abl inhibitors such as bosutinib; cyclin-dependent kinase (CDK) inhibitors such as palbociclib, PF-06873600; erb inhibitors such as dacomitinib; PARP inhibitors such as talazoparib; SMO inhibitors such as glasdegib, PF-5274857; EGFR T790M inhibitors such as PF-06747775; EZH2 inhibitors such as PF-06821497; PRMT5 inhibitors; TGFRBr1 inhibitors such as PF-06952229; and pharmaceutically acceptable salts, acids or derivatives of any of the above. Chemotherapeutic agents are typically small molecules.

In an embodiment of the treatment methods, medicaments and uses of the present invention, the VEGFR inhibitor is axitinib or AG-013736. Axitinib, as well as pharmaceutically acceptable salts thereof, is described in U.S. Pat. No. 6,534,524. Methods of making axitinib are described in U.S. Pat. Nos. 6,884,890 and 7,232,910, in U.S. Publication Nos. 2006-0091067 and 2007-0203196 and in International Publication No. WO 2006/048745. Dosage forms of axitinib are described in U.S. Publication No. 2004-0224988. Polymorphic forms and pharmaceutical compositions of axitinib are also described in U.S. Publication Nos. 2006-0094763, 2008-0274192 and 2010-0179329 and International Publication No. WO 2013/046133. The patents and patent applications listed above are incorporated herein by reference.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

In some aspects, the antibodies of the disclosure may be provided for use one or more selected from the group consisting of the inhibition of tumor growth; the inhibition of progression of malignant cell growth in a patient; the inhibition of metastasis of malignant cells in a patient; the induction of tumor regression in a patient; and the treatment of a cancer presenting with a solid tumor.

In some aspects, the use is for the treatment of one or more selected from the group consisting of bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

In some aspects, the use is for the treatment of one or more selected from the group consisting of renal cell carcinoma (RCC), bladder cancer, breast cancer, clear cell kidney cancer, head/neck squamous cell carcinoma (SCCHN), lung squamous cell carcinoma, malignant melanoma, non-small-cell lung cancer (NSCLC), ovarian cancer, pancreatic cancer, prostate cancer, small-cell lung cancer (SCLC) or triple negative breast cancer. In some aspects, the use is for the treatment of one or more selected from the group consisting of a Heme malignancy and in some embodiments, the Heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).

In some aspects, the disclosure provides a method for treating a cancer in a subject comprising administering to the subject a combination therapy which comprises a first anti-cancer therapeutic agent and a second anti-cancer therapeutic agent, wherein the first anti-cancer therapeutic agent is the antibody against one or more of IL-4, IL-13, and TSLP, and wherein the second anti-cancer therapeutic agent is selected from the group consisting of an anti-OX40 antibody, an anti-4-1 BB antibody, an anti-HER2 antibody, a PD-1 pathway antagonist, an anti-PD-1 antibody, an anti-PD-L1 antibody, a TLR3 agonist, a TLR 7/8 agonist, a TLR9 agonist, a bispecific anti-CD47/anti-PD-L1 antibody, and a bispecific anti-P-cadherin/anti-CD3 antibody.

In some aspects, the second anti-cancer therapeutic agent is a PD-1 antagonist, and the PD-1 antagonist is selected from the group consisting of sasanlimab, BCD-100, camrelizumab, cemiplimab, genolimzumab, MEDI0680, nivolumab, pembrolizumab, sintilimab, spartalizumab, STI-A1110, tislelizumab, atezolizumab, durvalumab, BMS-936559 (MDX-1105), LY3300054, TSR-042.

In some aspects, the second anti-cancer therapeutic agent comprises a VH as shown in SEQ ID NO: 4 and a VL as shown in SEQ ID NO: 8 of U.S. Ser. No. 10/155,037, and more favorably, comprises a HC comprising a sequence according to SEQ ID NO; 225 and a light chain comprising a sequence according to SEQ ID NO: 226.

The disclosure provides a method for treating a cancer in a subject comprising administering to the subject a combination therapy which comprises a first anti-cancer therapeutic agent and a second anti-cancer therapeutic agent, wherein the first anti-cancer therapeutic agent is selected from the group consisting of IL413TSLP-1024, IL413TSLP-1028, and IL413TSLP-1037, and the second anti-cancer therapeutic is a PD-1 antagonist, such as sasanlimab.

### Multispecific Antibody Formats

In some aspects, provided herein is an antibody that comprises a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site.

In some aspects, the first and second polypeptide chains associate together to form an antibody comprising two arms; a dual Fab arm comprising the first Fab domain and the second Fab domain, and a single Fab arm comprising the third Fab domain.

In some aspects, the first Fab comprises a first antigen associated VH (VH-1), a first antigen associated VL (VL-1), a first antigen associated CL (CL-1), and a first antigen associated CH1 (CH1-1). In some aspects, the C-terminus of the VH-1 is covalently fused by a peptide bond to the N-terminus of the CH1-1. In some aspects, the C-terminus of the VL-1 is covalently fused by a peptide bond to the N-terminus of the CL-1. In some aspects, the C-terminus of the VH-1 is covalently fused by a peptide bond to the N-terminus of the CL-1. In some aspects, the C-terminus of the VL-1 is covalently fused by a peptide bond to the N-terminus of the CH1-1.

In some aspects, the second Fab comprises a second antigen associated VH (VH-2), a second antigen associated VL (VL-2), a second antigen associated CL (CL-2), and a second antigen associated CH1 (CH1-2). In some aspects, the C-terminus of the VH-2 is covalently fused by a peptide bond to the N-terminus of the CH1-2. In some aspects, the C-terminus of the VL-2 is covalently fused by a peptide bond to the N-terminus of the CL-2. In some aspects, the C-terminus of the VH-2 is covalently fused by a peptide bond to the N-terminus of the CL-2. In some aspects, the C-terminus of the VL-2 is covalently fused by a peptide bond to the N-terminus of the CH1-2.

In some aspects, the third Fab comprises a third antigen associated VH (VH-3), a first antigen associated VL (VL-3), a first antigen associated CL (CL-3), and a first antigen associated CH1 (CH1-3). In some aspects, the C-terminus of the VH-3 is covalently fused by a peptide bond to the N-terminus of the CH1-3. In some aspects, the C-terminus of the VL-3 is covalently fused by a peptide bond to the N-terminus of the CL-3. In some aspects, the C-terminus of the VH-3 is covalently fused by a peptide bond to the N-terminus of the CL-3. In some aspects, the C-terminus of the VL-3 is covalently fused by a peptide bond to the N-terminus of the CH1-3.

In some aspects, the second polypeptide comprises from N-terminus to C-terminus, (VL-1)-(CL-1)-(linker)-(VH-2)-(CH1-2)-(second hinge)-(second CH2)-(second CH3); the fifth polypeptide comprises from N-terminus to C-terminus, (VH1)-(CL-1); and the fourth polypeptide comprises (VL-2)-(CL-2). The first polypeptide may comprise, from N-terminus to C-terminus, (VH-3)-(CH1-3)-(first hinge)-(first CH2)-(first CH3); and the third polypeptide may comprise (VL-3)-(CL-3).

In some aspects, the second polypeptide comprises from N-terminus to C-terminus, (VH-1)-(CH1-1)-(linker)-(VH-2)-(CH1-2)-(second hinge)-(second CH2)-(second CH3); the fifth polypeptide comprises from N-terminus to C-terminus, (VL1)-(CL-1); and the fourth polypeptide comprises (VL-2)-(CL-2). The first polypeptide may comprise, from N-terminus to C-terminus, (VH-3)-(CH1-3)-(first hinge)-(first CH2)-(first CH3); and the third polypeptide may comprise (VL-3)-(CL-3).

In some aspects, the second polypeptide comprises from N-terminus to C-terminus, (VL-1)-(CL-1)-(linker)-(VL-2)-(CH1-2)-(second hinge)-(second CH2)-(second CH3); the fifth polypeptide comprises from N-terminus to C-terminus, (VH1)-(CH1-1); and the fourth polypeptide comprises (VH-2)-(CL-2). The first polypeptide may comprise, from N-terminus to C-terminus, (VH-3)-(CH1-3)-(first hinge)-(first CH2)-(first CH3); and the third polypeptide may comprise (VL-3)-(CL-3).

In some aspects, the second polypeptide comprises from N-terminus to C-terminus, (VL-1)-(CH1-1)-(linker)-(VH-2)-(CH1-2)-(second hinge)-(second CH2)-(second CH3); the fifth polypeptide comprises from N-terminus to C-terminus, (VH1)-(CL-1); and the fourth polypeptide comprises (VL-2)-(CL-2). The first polypeptide may comprise, from N-terminus to C-terminus, (VH-3)-(CH1-3)-(first hinge)-(first CH2)-(first CH3); and the third polypeptide may comprise (VL-3)-(CL-3).

In some aspects, the fifth polypeptide comprises the sequence EPKSC (SEQ ID NO: 122) at the C-terminus.

In some aspects, the CH1-1 domain may comprise a sequence selected from the group consisting of SEQ ID NO: 6, SEQ ID NO: 105, and SEQ ID NO: 110. In some aspects, the CH1-2 domain may comprise a sequence selected from the group consisting of SEQ ID NO: 6, SEQ ID NO: 105, and SEQ ID NO: 110. In some aspects, the CH1-3 domain may comprise a sequence selected from the group consisting of SEQ ID NO: 6, SEQ ID NO: 105, and SEQ ID NO: 110.

The CL-1 may be a constant light kappa domain. The CL-2 may be a constant light kappa domain. The CL-3 may be a constant light kappa domain. The CL-1 may be a constant light lambda domain. The CL-2 may be a constant light lambda domain. The CL-3 may be a constant light lambda domain.

In some aspects, the CL-1 domain comprises a sequence selected from the group consisting of SEQ ID NO: 16, SEQ ID NO: 95, SEQ ID NO: 108, and SEQ ID NO: 113. In some aspects, the CL-2 domain comprises a sequence selected from the group consisting of SEQ ID NO: 16, SEQ ID NO: 95, SEQ ID NO: 108, and SEQ ID NO: 113. In some aspects, the CL-3 domain comprises a sequence selected from the group consisting of SEQ ID NO: 16, SEQ ID NO: 95, SEQ ID NO: 108, and SEQ ID NO: 113.

In some aspects, the first hinge comprises a sequence selected from the group consisting of SEQ ID NO: 7, SEQ ID NO: 102, SEQ ID NO: 123, SEQ ID NO: 126, SEQ ID NO: 129, and SEQ ID NO: 131. In some aspects, the second hinge comprises a sequence selected from the group consisting of SEQ ID NO: 7, SEQ ID NO: 102, SEQ ID NO: 123, SEQ ID NO: 126, SEQ ID NO: 129, and SEQ ID NO: 131. In some aspects, the first hinge region and the second hinge region comprise a pair of sequences according to SEQ ID NO: 129 and SEQ ID NO: 131.

In some aspects, one or both of the first CH2 domain and the second CH2 domain comprises a sequence according to SEQ ID NO: 8.

In some aspects, the first CH3 domain and the second CH3 domain each comprise a different and complementary sequence, and the different and complementary sequences are selected from one of the following pairs of different and complementary sequences:
(i) SEQ ID NO: 111 and SEQ ID NO: 106;
(ii) SEQ ID NO: 111 and SEQ ID NO: 114;
(iii) SEQ ID NO: 114 and SEQ ID NO: 117;
(iv) SEQ ID NO: 124 and SEQ ID NO: 127;
(v) SEQ ID NO: 139 and SEQ ID NO: 141; and
(vi) SEQ ID NO: 147 and SEQ ID NO: 148.

In some aspects,
(i) the CL-1 comprises a sequence according to SEQ ID NO: 16, the linker comprises a sequence according to SEQ ID NO: 104, the CH1-2 comprises a sequence according to SEQ ID NO: 6, the second hinge comprises a sequence according to SEQ ID NO: 129, the second CH2 comprises a sequence according to SEQ ID NO: 8, the second CH3 comprises a sequence according to SEQ ID NO: 124,
(ii) the CH1-1 comprises a sequence according to SEQ ID NO: 6,
(iii) the CL-2 comprises a sequence according to SEQ ID NO: 16;
In some aspects, the CL-3 comprises a sequence according to SEQ ID NO: 95. In some aspects, the CH1-3 comprises a sequence according to SEQ ID NO: 6. In some aspects, the first hinge comprises a sequence according to SEQ ID NO: 131. In some aspects, the first CH2 comprises a sequence according to SEQ ID NO: 8. In some aspects, the first CH3 comprises a sequence according to SEQ ID NO:127.

### Antibodies of the Disclosure

One or more of the antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, the antibody is a monoclonal antibody. In some embodiments, the antibody is a human or humanized antibody. In some embodiments, the antibody is a chimeric antibody.

The invention encompasses modifications to the CDRs, VH, VL, HC, and LC regions shown in one or more of Tables 80, 81, 82, 83, 84, 85, 86, and 87. For example, the invention includes antibodies comprising functionally equivalent variable regions and CDRs which do not significantly affect their properties as well as variants which have enhanced or decreased activity or affinity. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

A modification or mutation may also be made in a framework region or constant region to increase the half-life of an antibody provided herein. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

In some aspects, antibodies of the disclosure comprise L247A, L248A and G250A (Kabat) or L234A L235A and G237A (EU) to minimize effector function. In some aspects, antibodies of the invention comprise the following mutations to extend half-life: M459L and N465S (Kabat) or M428L and N434S (EU). In some aspects, the antibodies of the disclosure comprise wild type residues at positions M459 and N465 (Kabat) or M428 and N434 (EU). Accordingly, the disclosure also provides antibodies as described herein comprising CH3 domains, wherein polypeptides comprising said CH3 domains differ from the defined SEQ ID for the polypeptide by a reversion to wild type residues at M459 and N465 (Kabat) or M428 and N434 (EU).

In some aspects, antibodies of the disclosure comprise mutations to facilitate in vitro heterodimerization of the heavy chains at D232R, K440R (Kabat) or D221R, K409R (EU), and D232E, K391E (Kabat) or D221E, L368E (EU) on respectively paired heavy chains. The disclosure also provides bispecific antibodies that surprisingly may be made with only two RR/EE mutations: at D(H232) in the hinge region, and K(H440) in the CH3 region. Residue P228 may be unmutated. The disclosure provides antibodies comprising an antibody Fc domain comprising a first Fc chain and a second Fc chain, wherein the first Fc chain and the second Fc chain each contain two amino acid modifications that promote the association of the first Fc chain with the second Fc chain, characterized in that (i) the first Fc chain comprises D(H232)R and K(H440)R, and the second Fc chain comprises D(H232)E and L(H391)E; or the first Fc chain comprises D(H232)E and K(H440)R, and the second Fc chain comprises L(H391)R and D(H232)E. Antibodies are provided wherein the first Fc chain comprises, in N-terminal to C-terminal order, a first hinge region connected to a first CH2 region which is connected to a first CH3 region, and herein the second Fc chain comprises, in N-terminal to C-terminal order, a second hinge region connected to a second CH2 region which is connected to a second CH3 region, and wherein the first hinge region and second hinge region comprise a pair of sequences according to SEQ ID NO: 129 and SEQ ID NO: 131, and the first CH3 region and the second CH3 region comprise either of the following two pairs pair of sequences: SEQ ID NO: 124 and SEQ ID NO: 127; or SEQ ID NO: 147 and SEQ ID NO: 148.

The disclosure also provides bispecific antibodies that surprisingly may be made with only one R/E mutation: K(H440) in the CH3 region. The disclosure provides antibodies comprising an antibody Fc domain comprising a first Fc chain and a second Fc chain, wherein the first Fc chain and the second Fc chain each contain one amino acid modification that promotes the association of the first Fc chain with the second Fc chain, characterized in that (i) the first Fc chain comprises K(H440)R, and the second Fc chain comprises L(H391)E. Antibodies are provided wherein the first Fc chain comprises, in N-terminal to C-terminal order, a first hinge region connected to a first CH2 region which is connected to a first CH3 region, and herein the second Fc chain comprises, in N-terminal to C-terminal order, a second hinge region connected to a second CH2 region which is connected to a second CH3 region, and wherein the first CH3 region and the second CH3 region comprise the pair of sequences: SEQ ID NO: 124 and SEQ ID NO: 127. Such antibodies are exemplified by IL13433-1261.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase Ill (GnTIII), a glycosyltransferase catalyzing formation of bisecting GlcNAc, was reported to have improved ADCC activity (Umana et al., 1999, Nature Biotech. 17:176-180).

The invention also encompasses fusion proteins comprising one or more components of the antibodies disclosed herein. In some embodiments, a fusion protein may be made that comprises all or a portion of an antibody of the invention linked to another polypeptide. In another embodiment, only the variable domains of the antibody are linked to the polypeptide. In another embodiment, the VH domain of an antibody is linked to a first polypeptide, while the VL domain of an antibody is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

### Polynucleotides Encoding Antibodies, and Methods of Manufacture

The disclosure also provides polynucleotides encoding any of the antibodies of the invention, including antibody portions and modified antibodies described herein. The invention also provides a method of making any of the antibodies and polynucleotides described herein. Polynucleotides can be made and the proteins expressed by procedures known in the art.

If desired, an antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide comprising a sequence encoding one or both of the heavy chain or the light chain variable regions of an antibody provided herein. The sequence encoding the antibody of interest may be maintained in a vector in a host cell and the host cell can then be expanded and frozen for future use. Vectors (including expression vectors) and host cells are further described herein. In some embodiments, the disclosure provides polynucleotides encoding the amino acid sequences of any of the antibodies listed in one or more of Tables 80, 81, 82, 83, 84, 85, 86, and 87. In one embodiment, the invention provides polynucleotides encoding the amino acid sequence of IL41333-1258; IL413TSLP-1024; or IL413p40-0705.

In some embodiments, the disclosure provides polynucleotides encoding one or more anti-IL-4 antibody heavy chain polypeptides comprising an amino acid sequence selected from the group consisting of: SEQ ID NOs: 23, 106, 115, 121, 124, 125, 130, 133, 135, 140, 144, 146, 148, 151, 156, 159, 162, 179, 180, and 183.

In some embodiments, the disclosure provides polynucleotides encoding one or more anti-IL-4 antibody light chain polypeptides comprising an amino acid sequence selected from the group consisting of: SEQ ID NOs: 27, 109, 116, 136, 197, 207, and 208.

In some embodiments, the disclosure provides polynucleotides encoding one or more anti-IL-4 antibody VH polypeptides comprising an amino acid sequence selected from the group consisting of: SEQ ID NOs: 19, 22, and 28.

In some embodiments, the disclosure provides polynucleotides encoding one or more anti-IL-4 antibody VL polypeptides comprising an amino acid sequence selected from the group consisting of: SEQ ID NOs: 20, 26, 29, and 30.

In some embodiments, the disclosure provides polynucleotides encoding one or more anti-IL-13 antibody heavy chain polypeptides comprising an amino acid sequence selected from the group consisting of: SEQ ID NOs: 52, 66, 112, 118, 121, 122, 145, 149, 152, 154, 160, 162, and 209.

In some embodiments, the disclosure provides polynucleotides encoding one or more anti-IL-13 antibody light chain polypeptides comprising an amino acid sequence selected from the group consisting of: SEQ ID NOs: 55. 119, 120, 125, 130, 133, 135, 140, 144, 163, 164, 180, and 196.

In some embodiments, the disclosure provides polynucleotides encoding one or more anti-IL-13 antibody VH polypeptides comprising an amino acid sequence selected from the group consisting of: SEQ ID NOs: 44, 48, 51, 57, and 65.

In some embodiments, the disclosure provides polynucleotides encoding one or more anti-IL-13 antibody VL polypeptides comprising an amino acid sequence selected from the group consisting of: SEQ ID NOs: 46, 49, 54, 58, 59, and 68.

In some embodiments, the disclosure provides polynucleotides encoding one or more anti-IL-33 antibody heavy chain polypeptides comprising an amino acid sequence selected from the group consisting of: SEQ ID NOs: 64, 74, 103, 128, 132, 134, 137, 142, and 143.

In some embodiments, the disclosure provides polynucleotides encoding one or more anti-IL-33 antibody light chain polypeptides comprising an amino acid sequence selected from the group consisting of: SEQ ID NOs: 79, 145, 107, 115, 121, 138, and 144 In some embodiments, the disclosure provides polynucleotides encoding one or more anti-IL-33 antibody VH polypeptides comprising an amino acid sequence selected from the group consisting of: SEQ ID NOs: 63, 73, and 80.

In some embodiments, the disclosure provides polynucleotides encoding one or more anti-IL-33 antibody VL polypeptides comprising an amino acid sequence selected from the group consisting of: SEQ ID NOs: 54, 68, 71, 78, and 81.

In some embodiments, the disclosure provides polynucleotides encoding one or more anti-TSLP antibody heavy chain polypeptides comprising an amino acid sequence selected from the group consisting of: SEQ ID NOs: 97, 155, 149, 151, 152, 153, 158, 159, 161, and 165.

In some embodiments, the disclosure provides polynucleotides encoding one or more anti-TSLP antibody light chain polypeptides comprising an amino acid sequence selected from the group consisting of: SEQ ID NOs: 98, 99, 150, 154, 156, 157, and 160.

In some embodiments, the disclosure provides polynucleotides encoding one or more anti-TSLP antibody VH polypeptides comprising an amino acid sequence selected from the group consisting of: SEQ ID NOs: 92.

In some embodiments, the disclosure provides polynucleotides encoding one or more anti-TSLP antibody VL polypeptides comprising an amino acid sequence selected from the group consisting of: SEQ ID NOs: 94.

The disclosure provides an isolated polynucleotide encoding the VH, VL, or both, of an antibody that binds IL-33, wherein the nucleic acid comprises: the nucleic acid sequence of SEQ ID NO: 202, the nucleic acid sequence of SEQ ID NO: 203, or both. The disclosure provides an isolated polynucleotide encoding a VH bearing polypeptide and a VL bearing polypeptide, or both, of an antibody that binds to IL-33, wherein said nucleic acid comprises the nucleic acid sequence of SEQ ID NO: 190, the nucleic acid sequence of SEQ ID NO: 191, or both. The disclosure provides an isolated polynucleotide encoding the VH, VL, or both, of an antibody that binds to IL-33, wherein said nucleic acid comprises the nucleic acid sequence of the insert of the plasmid deposited with the ATCC and having the Accession Number PTA-127209 the nucleic acid sequence of the insert of the plasmid deposited with the ATCC and having the Accession Number PTA-127210 or both. The disclosure provides an isolated polynucleotide encoding a VH bearing polypeptide and a VL bearing polypeptide, or both, of an antibody that binds to IL-33, wherein said nucleic acid comprises the nucleic acid sequence of the insert of the plasmid deposited with the ATCC and having the Accession Number PTA-127207, the nucleic acid sequence of the insert of the plasmid deposited with the ATCC and having the Accession Number PTA-127208, or both.

The disclosure provides an isolated polynucleotide encoding the VH, VL, or both, of an antibody that binds TSLP, wherein the nucleic acid comprises: the nucleic acid sequence of SEQ ID NO: 204, the nucleic acid sequence of SEQ ID NO: 205, or both. The disclosure provides an isolated polynucleotide encoding a VH bearing polypeptide and a VL bearing polypeptide, or both, of an antibody that binds to TSLP, wherein said nucleic acid comprises the nucleic acid sequence of SEQ ID NO: 192, the nucleic acid sequence of SEQ ID NO: 193, or both. The disclosure provides an isolated polynucleotide encoding the VH, VL, or both, of an antibody that binds to TSLP, wherein said nucleic acid comprises the nucleic acid sequence of the insert of the plasmid deposited with the ATCC and having the Accession Number PTA-127200 the nucleic acid sequence of the insert of the plasmid deposited with the ATCC and having the Accession Number PTA-127199 or both. The disclosure provides an isolated polynucleotide encoding a VH bearing polypeptide and a VL bearing polypeptide, or both, of an antibody that binds to TSLP, wherein said nucleic acid comprises the nucleic acid sequence of the insert of the plasmid deposited with the ATCC and having the Accession Number PTA-127202, the nucleic acid sequence of the insert of the plasmid deposited with the ATCC and having the Accession Number PTA-127201, or both.

The disclosure provides an isolated polynucleotide encoding a VH bearing polypeptide and a VL bearing polypeptide, or both, of an antibody that binds to p40, wherein said nucleic acid comprises the nucleic acid sequence of SEQ ID NO: 194, the nucleic acid sequence of SEQ ID NO: 195, or both. The disclosure provides an isolated polynucleotide encoding a VH bearing polypeptide and a VL bearing polypeptide, or both, of an antibody that binds to p40, wherein said nucleic acid comprises the nucleic acid sequence of the insert of the plasmid deposited with the ATCC and having the Accession Number PTA-127204, the nucleic acid sequence of the insert of the plasmid deposited with the ATCC and having the Accession Number PTA-127203, or both.

The disclosure provides an isolated polynucleotide encoding the VH, VL, or both, of an antibody that binds IL-4, wherein the nucleic acid comprises: the nucleic acid sequence of SEQ ID NO: 200, the nucleic acid sequence of SEQ ID NO: 201, or both. The disclosure provides an isolated polynucleotide encoding a VH bearing polypeptide and a VL bearing polypeptide, or both, of an antibody that binds to IL-4, wherein said nucleic acid comprises the nucleic acid sequence of SEQ ID NO: 188, the nucleic acid sequence of SEQ ID NO: 189, or both. The disclosure provides an isolated polynucleotide encoding the VH, VL, or both, of an antibody that binds to IL-4, wherein said nucleic acid comprises the nucleic acid sequence of the insert of the plasmid deposited with the ATCC and having the Accession Number PTA-127198 the nucleic acid sequence of the insert of the plasmid deposited with the ATCC and having the Accession Number PTA-127197 or both. The disclosure provides an isolated polynucleotide encoding a VH bearing polypeptide and a VL bearing polypeptide, or both, of an antibody that binds to IL-4, wherein said nucleic acid comprises the nucleic acid sequence of the insert of the plasmid deposited with the ATCC and having the Accession Number PTA-127192, the nucleic acid sequence of the insert of the plasmid deposited with the ATCC and having the Accession Number PTA-127194, or both.

The disclosure provides an isolated polynucleotide encoding the VH, VL, or both, of an antibody that binds IL-13, wherein the nucleic acid comprises: the nucleic acid sequence of SEQ ID NO: 196, the nucleic acid sequence of SEQ ID NO: 195, or both. The disclosure provides an isolated polynucleotide encoding a VH bearing polypeptide and a VL bearing polypeptide, or both, of an antibody that binds to IL-13, wherein said nucleic acid comprises the nucleic acid sequence of SEQ ID NO: 187, the nucleic acid sequence of SEQ ID NO: 188, or both. The disclosure provides an isolated polynucleotide encoding the VH, VL, or both, of an antibody that binds to IL-13, wherein said nucleic acid comprises the nucleic acid sequence of the insert of the plasmid deposited with the ATCC and having the Accession Number PTA-127196 the nucleic acid sequence of the insert of the plasmid deposited with the ATCC and having the Accession Number PTA-127195 or both. The disclosure provides an isolated polynucleotide encoding a VH bearing polypeptide and a VL bearing polypeptide, or both, of an antibody that binds to IL-13, wherein said nucleic acid comprises the nucleic acid sequence of the insert of the plasmid deposited with the ATCC and having the Accession Number PTA-127193, the nucleic acid sequence of the insert of the plasmid deposited with the ATCC and having the Accession Number PTA-127192, or both.

The disclosure provides an isolated polynucleotide encoding one or more of the first, second, third, fourth, or fifth polypeptides of an anti-IL-4/IL-13/IL-33 antibody, comprising
(i) an IL33-VH sequence encoded by the plasmid deposited at the ATCC and having ATCC Accession No. PTA-127210, and an IL33-VL sequence encoded by the plasmid deposited at the ATCC and having ATCC Accession No. PTA-127209;
(ii) an IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

The disclosure provides an isolated polynucleotide encoding one or more of the first, second, third, fourth, or fifth polypeptides of an anti-IL-4/IL-13/IL-33 antibody, wherein the isolated antibody specifically binds IL-33, that specifically binds to IL-4, and that specifically binds to IL-13, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an IL-33 binding site, and
wherein the first polypeptide chain comprises a sequence encoded by the plasmid deposited at the ATCC and having ATCC Accession No. PTA-127208; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127207; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

The disclosure provides an isolated polynucleotide encoding one or more of the first, second, third, fourth, or fifth polypeptides of an anti-IL-4/IL-13/TSLP antibody, wherein the antibody comprises
(i) a TSLP-VH sequence encoded by the plasmid deposited at the ATCC and having ATCC Accession No. PTA-127200, and a TSLP-VL sequence encoded by the plasmid deposited at the ATCC and having ATCC Accession No. PTA0-127199.
(ii) a IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

The disclosure provides an isolated polynucleotide encoding one or more of the first, second, third, fourth, or fifth polypeptides of an anti-IL-4/IL-13/TSLP antibody, wherein the antibody comprises a first, second, third, fourth, and fifth polypeptide chain and wherein
(i) the second and fifth polypeptide chains together form a first Fab domain comprising a IL-13 binding site;
(ii) the second and fourth polypeptide chains together form a second Fab domain comprising an IL-4 binding site;
(iii) the first and third polypeptide chains together form a third Fab domain comprising a TSLP binding site, and
wherein the first polypeptide chain comprises a sequence encoded by the plasmid deposited at the ATCC and having ATCC Accession No. PTA-127202; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127201; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

The disclosure provides an isolated polynucleotide encoding one or more of the first, second, third, fourth, or fifth polypeptides of an anti-IL-4/IL-13/p40 antibody, wherein the antibody comprises
(i) a p40-VH sequence encoded by the plasmid deposited at the ATCC and having ATCC Accession No. PTA-127206, and a p40-VL sequence encoded by the plasmid deposited at the ATCC and having ATCC Accession No. PTA-127205.
(ii) an IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197; and
(iii) an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195.

The disclosure provides an isolated polynucleotide encoding one or more of the first, second, third, fourth, or fifth polypeptides of an anti-IL-4/IL-13/p40 antibody, wherein the antibody comprises a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising a p40 binding site, and
wherein the first polypeptide chain comprises a sequence encoded by the plasmid deposited at the ATCC and having ATCC Accession No. PTA-127204; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127203; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, CoIE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

### Pharmaceutical Compositions

In other embodiments, the invention comprises pharmaceutical compositions.

A “pharmaceutical composition” refers to a mixture of an antibody the invention and one or excipient. As used herein, pharmaceutical compositions may comprise one or more antibodies that bind to one or more of IL-4, IL-13, IL-33, TSLP, and p40, one or more antibodies that bind to IL-4 and IL-13, and one or more antibodies that bind to IL-4/IL-13/IL-33, or IL-4/IL-13/TSLP, or IL-4/IL-13/p40, or one or more polynucleotides comprising sequences encoding one or more these antibodies. These compositions may further comprise suitable excipients, such as pharmaceutically acceptable excipients including buffers, which are well known in the art.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application. Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

The invention provided herein further encompasses methods and compositions for treatment, prevention or management of one or more disorders selected from the group consisting of an IL-4 related disorder, and IL-13 related disorder, and IL-33 related disorder, a TSLP related disorder, a p40 related disorder, inflammatory disorders, atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa, in a subject, comprising administering to the subject a therapeutically effective amount of an anti-IL-4, anti-IL-13, anti-IL-33, anti-TSLP, anti-IL4/IL-13 multispecific, IL-4/IL-13/IL-33; IL-4/IL-13/TSLP; or IL-4/IL-13/p40 antibody provided herein.

In one aspect, the invention provides a method for treating a condition associated with one or more of IL-4, IL-13, IL-33, TSLP, and p40 expression in a subject. In some embodiments, the method of treating a condition associated with one or more of IL-4, IL-13, IL-33, TSLP, and p40 expression in a subject comprises administering to the subject in need thereof an effective amount of a composition (e.g., pharmaceutical composition) comprising the respective anti-IL-4, anti-IL-13, anti-IL-33, anti-TSLP, IL-4/IL-13, IL-4/IL-13/IL-33, IL-4/IL-13/TSLP, or IL-4/IL-13/p40 multispecific antibodies as described herein. The conditions associated with IL-4, IL-13, IL-33, TSLP, and p40 expression include, but are not limited to, abnormal expression of one or more of IL-4, IL-13, IL-33, TSLP, and p40 expression, altered or aberrant IL-4, IL-13, IL-33, TSLP, or p40 expression.

In one aspect, the present invention provides one or more selected from the group consisting of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies described herein, or a pharmaceutical composition comprising such antibody for use in therapy. In a particular embodiment, the invention also provides one or more of anti-IL-4, anti-IL-13, anti-IL-33, anti-TSLP, anti-IL-4/IL-13, anti-IL-4/IL-13/IL-33, anti-IL-4/IL-13/TSLP, anti-IL-4/IL-13/p40 antibodies for use in treating a disorder associated with one more of IL-4, IL-13, IL-33, TSALP, p40, IL-4/IL-13, IL-4/IL-13/IL-33, IL-4/IL-13/TSLP, and IL-4/IL-13/p40.

The present invention further provides one or more selected from the group consisting of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies as described herein, or a pharmaceutical composition comprising such antibody for use in the manufacture of a medicament for use in therapy. In some embodiments, the therapy is a treatment of a disorder associated with one or more of IL-4, IL-13, IL-33, TSALP, p40, IL-4/IL-13, IL-4/IL-13/IL-33, IL-4/IL-13/TSLP, and IL-4/IL-13/p40.

### Therapeutic, Diagnostic, and Other Methods

The antibodies and the antibody conjugates of the present invention are useful in various applications including, but are not limited to, therapeutic treatment methods and diagnostic treatment methods.

IL-4 antibodies of the invention may inhibit the activity of IL-4 and may be useful in the treatment, prevention, suppression and amelioration of IL-4 related diseases. The invention provides a method for treating disorders associated with IL-4 expression. The invention provides a method of treating one or more of the disorders selected from the group consisting of atopic dermatitis, atopic dermatitis, asthma, cancer, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, Crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa in a subject comprising administering to the subject in need thereof an effective amount of a pharmaceutical composition comprising any of the IL-4 antibodies as described herein. The preceding sentence provides a list of disorders associated with IL-4 expression.

In some aspects, IL-4 antibodies of the invention may inhibit the activity of IL-4 and may be useful in the treatment, prevention, suppression and amelioration of one or more diseases selected from the group consisting of atopic dermatitis, asthma, cancer, COPD, food allergy, allergic rhinitis, eosinophilic esophagitis, chronic rhinosinusitis with nasal polyps, alopecia areata, non-alcoholic steatohepatitis (NASH), alopecia, idiopathic pulmonary fibrosis (IPF), and systemic sclerosis. In some aspects, IL-4 antibodies of the invention may be useful in the treatment, prevention, suppression and amelioration of atopic dermatitis. In some aspects, IL-4 antibodies of the invention may be useful in the treatment, prevention, suppression and amelioration of asthma. In some aspects, IL-4 antibodies of the invention may be useful in the treatment, prevention, suppression and amelioration of NASH.

In another aspect, the invention further provides an antibody or pharmaceutical composition as described herein for use in the described method of treating one or more of the disorders associated with IL-4 expression. The invention also provides the use of an antibody as described herein in the manufacture of a medicament for treating one or more disorders associated with IL-4 expression. In another aspect, provided is a method of one or more of detecting, diagnosing, or monitoring one or more of the disorders associate with IL-4 expression. For example, the anti-IL-4 antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent. With respect to all methods described herein, reference to anti-IL-4 antibodies also includes pharmaceutical compositions comprising the anti-IL-4 antibodies and one or more additional agents.

IL-13 antibodies of the invention may inhibit the activity of IL-13 and may be useful in the treatment, prevention, suppression and amelioration of IL-13 related diseases. The invention provides a method for treating disorders associated with IL-13 expression. The invention provides a method of treating one or more of the disorders selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, and systemic sclerosis, diabetic kidney disease, Behcet's disease, gout, Alzheimer's disease, atherosclerosis, fungal keratitis, non-alcoholic steatohepatitis (NASH), psoriasis, psoriatic arthritis, Crohn's disease, ulcerative colitis, allergy, alopecia, idiopathic pulmonary fibrosis, systemic sclerosis, keloids, systemic lupus erythematosus (SLE), primary biliary cirrhosis, and hidradenitis suppurativa in a subject comprising administering to the subject in need thereof an effective amount of a pharmaceutical composition comprising any of the IL-13 antibodies as described herein. The preceding sentence provides a list of disorders associated with IL-13 expression.

In some aspects, IL-13 antibodies of the invention may inhibit the activity of IL-13 and may be useful in the treatment, prevention, suppression and amelioration of one or more diseases selected from the group consisting of atopic dermatitis, asthma, COPD, food allergy, allergic rhinitis, eosinophilic esophagitis, chronic rhinosinusitis with nasal polyps, alopecia areata, non-alcoholic steatohepatitis (NASH), alopecia, idiopathic pulmonary fibrosis, and systemic sclerosis. In some aspects, IL-13 antibodies of the invention may be useful in the treatment, prevention, suppression and amelioration of atopic dermatitis. In some aspects, IL-13 antibodies of the invention may be useful in the treatment, prevention, suppression and amelioration of asthma. In some aspects, IL-13 antibodies of the invention may be useful in the treatment, prevention, suppression and amelioration of NASH.

In another aspect, the invention further provides an antibody or pharmaceutical composition as described herein for use in the described method of treating one or more of the disorders associated with IL-13 expression. The invention also provides the use of an antibody as described herein in the manufacture of a medicament for treating one or more disorders associated with IL-13 expression. In another aspect, provided is a method of one or more of detecting, diagnosing, or monitoring one or more of the disorders associate with IL-13 expression. For example, the anti-IL-13 antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent. With respect to all methods described herein, reference to anti-IL-13 antibodies also includes pharmaceutical compositions comprising the anti-IL-13 antibodies and one or more additional agents.

IL-33 antibodies of the invention may inhibit the activity of IL-33 and may be useful in the treatment, prevention, suppression and amelioration of IL-33 related diseases. The invention provides a method for treating disorders associated with IL-33 expression. The invention provides a method of treating one or more of the disorders selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, diabetic kidney disease, Behcet's disease, gout, Alzheimer's disease, and atherosclerosis in a subject comprising administering to the subject in need thereof an effective amount of a pharmaceutical composition comprising any of the IL-33 antibodies as described herein. The preceding sentence provides a list of disorders associated with IL-33 expression.

In some aspects, IL-33 antibodies of the invention may inhibit the activity of IL-33 and may be useful in the treatment, prevention, suppression and amelioration of one or more diseases selected from the group consisting of atopic dermatitis, asthma, COPD, food allergy, allergic rhinitis, Eosinophilic esophagitis, Chronic rhinosinusitis with nasal polyps, alopecia areata, and non-alcoholic steatohepatitis (NASH). In some aspects, IL-33 antibodies of the invention may be useful in the treatment, prevention, suppression and amelioration of atopic dermatitis. In some aspects, IL-33 antibodies of the invention may be useful in the treatment, prevention, suppression and amelioration of asthma. In some aspects, IL-33 antibodies of the invention may be useful in the treatment, prevention, suppression and amelioration of NASH. In another aspect, the invention further provides an antibody or pharmaceutical composition as described herein for use in the described method of treating one or more of the disorders associated with IL-33 expression. The invention also provides the use of an antibody as described herein in the manufacture of a medicament for treating one or more disorders associated with IL-33 expression. In another aspect, provided is a method of one or more of detecting, diagnosing, or monitoring one or more of the disorders associate with IL-33 expression. For example, the anti-IL-33 antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent. With respect to all methods described herein, reference to anti-IL-33 antibodies also includes pharmaceutical compositions comprising the anti-IL-33 antibodies and one or more additional agents.

TSLP antibodies of the invention may inhibit the activity of TSLP and may be useful in the treatment, prevention, suppression and amelioration of TSLP related diseases. The invention provides a method for treating disorders associated with TSLP pression. The invention provides a method of treating one or more of the disorders selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis in a subject comprising administering to the subject in need thereof an effective amount of a pharmaceutical composition comprising any of the TSLP antibodies as described herein. The preceding sentence provides a list of disorders associated with TSLP expression.

In some aspects, TSLP antibodies of the invention may inhibit the activity of TSLP and may be useful in the treatment, prevention, suppression and amelioration of one or more diseases selected from the group consisting of atopic dermatitis, asthma, COPD, food allergy, allergic rhinitis, eosinophilic esophagitis, chronic rhinosinusitis with nasal polyps, alopecia areata, and non-alcoholic steatohepatitis (NASH). In some aspects, TSLP antibodies of the invention may be useful in the treatment, prevention, suppression and amelioration of atopic dermatitis. In some aspects, TSLP antibodies of the invention may be useful in the treatment, prevention, suppression and amelioration of asthma. In some aspects, TSLP antibodies of the invention may be useful in the treatment, prevention, suppression and amelioration of NASH.

In another aspect, the invention further provides an antibody or pharmaceutical composition as described herein for use in the described method of treating one or more of the disorders associated with TSLP expression. The invention also provides the use of an antibody as described herein in the manufacture of a medicament for treating one or more disorders associated with TSLP expression. In another aspect, provided is a method of one or more of detecting, diagnosing, or monitoring one or more of the disorders associate with TSLP expression. For example, the anti-TSLP antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent. With respect to all methods described herein, reference to anti-TSLP antibodies also includes pharmaceutical compositions comprising the anti-TSLP antibodies and one or more additional agents.

IL-4/IL-13/IL-33 antibodies of the invention may inhibit the activity of IL-4, IL-13, and IL-33 and may be useful in the treatment, prevention, suppression and amelioration of IL-4, IL-13, and IL-33 related diseases. The invention provides a method for treating disorders associated with IL-4, IL-13, and IL-33 expression. The invention provides a method of treating one or more of the disorders selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, Bullous pemphigoid, chronic urticaria, IPF, scleroderma, systemic sclerosis, diabetic kidney disease, Behcet's disease, gout, Alzheimer's disease, and atherosclerosis in a subject comprising administering to the subject in need thereof an effective amount of a pharmaceutical composition comprising any of the IL-4/IL-13/IL-33 antibodies as described herein. The preceding sentence provides a list of disorders associated with IL-4/IL-13/IL-33 expression.

In some aspects, IL-4/IL-13/IL-33 antibodies of the invention may inhibit the activity of IL-4, IL-13, and IL-33 and may be useful in the treatment, prevention, suppression and amelioration of one or more diseases selected from the group consisting of atopic dermatitis, asthma, COPD, food allergy, allergic rhinitis, eosinophilic esophagitis, chronic rhinosinusitis with nasal polyps, and alopecia areata. IL-4/IL-13/IL-33 antibodies of the invention may be useful in the treatment, prevention, suppression and amelioration of asthma. IL-4/IL-13/IL-33 antibodies of the invention may be useful in the treatment, prevention, suppression and amelioration of atopic dermatitis. IL-4/IL-13/IL-33 antibodies of the invention may be useful in the treatment, prevention, suppression and amelioration of NASH.

In another aspect, the invention further provides an antibody or pharmaceutical composition as described herein for use in the described method of treating one or more of the disorders associated with IL-4/IL-13/IL-33 expression. The invention also provides the use of an antibody as described herein in the manufacture of a medicament for treating one or more disorders associated with IL-4/IL-13/IL-33 expression. In another aspect, provided is a method of one or more of detecting, diagnosing, or monitoring one or more of the disorders associate with IL-4/IL-13/IL-33 expression. For example, the anti-IL-4/IL-13/IL-33 antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent. With respect to all methods described herein, reference to anti-IL-4/IL-13/IL-33 antibodies also includes pharmaceutical compositions comprising the anti-IL-4/IL-13/IL-33 antibodies and one or more additional agents.

IL-4/IL-13/TSLP antibodies of the invention may inhibit the activity of IL-4, IL-13, and TSLP and may be useful in the treatment, prevention, suppression and amelioration of IL-4, IL-13, and TSLP related diseases. The invention provides a method for treating disorders associated with IL-4, IL-13, and TSLP expression. The invention provides a method of treating one or more of the disorders selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis in a subject comprising administering to the subject in need thereof an effective amount of a pharmaceutical composition comprising any of the IL-4/IL-13/TSLP antibodies as described herein. The preceding sentence provides a list of disorders associated with IL-4/IL-13/TSLP expression.

IL-4/IL-13/TSLP antibodies of the invention may inhibit the activity of IL-4, IL-13, and TSLP and may be useful in the treatment, prevention, suppression and amelioration of one or more diseases selected from the group consisting of atopic dermatitis, asthma, COPD, food allergy, allergic rhinitis, eosinophilic esophagitis, chronic rhinosinusitis with nasal polyps, and alopecia areata. IL-4/IL-13/TSLP antibodies of the invention may be useful in the treatment, prevention, suppression and amelioration of asthma. IL-4/IL-13/TSLP antibodies of the invention may be useful in the treatment, prevention, suppression and amelioration of atopic dermatitis. IL-4/IL-13/TSLP antibodies of the invention may be useful in the treatment, prevention, suppression and amelioration of NASH.

In another aspect, the invention further provides an antibody or pharmaceutical composition as described herein for use in the described method of treating one or more of the disorders associated with IL-4/IL-13/TSLP expression. The invention also provides the use of an antibody as described herein in the manufacture of a medicament for treating one or more disorders associated with IL-4/IL-13/TSLP expression. In another aspect, provided is a method of one or more of detecting, diagnosing, or monitoring one or more of the disorders associate with IL-4/IL-13/TSLP expression. For example, the anti-IL-4/IL-13/TSLP antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent. With respect to all methods described herein, reference to anti-IL-4/IL-13/TSLP antibodies also includes pharmaceutical compositions comprising the anti-IL-4/IL-13/TSLP antibodies and one or more additional agents.

IL-4/IL-13/p40 antibodies of the invention may inhibit the activity of IL-4, IL-13, and p40 and may be useful in the treatment, prevention, suppression and amelioration of IL-4, IL-13, and p40 related diseases. The invention provides a method for treating disorders associated with IL-4, IL-13, and p40 expression. The invention provides a method of treating one or more of the disorders selected from the group consisting of non-alcoholic steatohepatitis (NASH), psoriasis, psoriatic arthritis, atopic dermatitis, Crohn's disease, ulcerative colitis, asthma (severe), allergy, alopecia, idiopathic pulmonary fibrosis, systemic sclerosis, keloids, systemic lupus erythematosus, primary biliary cirrhosis, and hidradenitis suppurativa in a subject comprising administering to the subject in need thereof an effective amount of a pharmaceutical composition comprising any of the IL-4/IL-13/p40 antibodies as described herein. The preceding sentence provides a list of disorders associated with IL-4/IL-13/p40 expression.

In some aspects, IL-4/IL-13/p40 antibodies of the invention may inhibit the activity of IL-4, IL-13, and p40 and may be useful in the treatment, prevention, suppression and amelioration of one or more diseases selected from the group consisting of non-alcoholic steatohepatitis (NASH), atopic dermatitis, asthma (severe), alopecia, idiopathic pulmonary fibrosis, and systemic sclerosis. In some aspects, IL-4/IL-13/p40 antibodies of the invention may be useful in the treatment, prevention, suppression and amelioration of atopic dermatitis. In some aspects, IL-4/IL-13/p40 antibodies of the invention may be useful in the treatment, prevention, suppression and amelioration of asthma. In some aspects, IL-4/IL-13/p40 antibodies of the invention may be useful in the treatment, prevention, suppression and amelioration of NASH.

In another aspect, the invention further provides an antibody or pharmaceutical composition as described herein for use in the described method of treating one or more of the disorders associated with IL-4/IL-13/p40 expression. The invention also provides the use of an antibody as described herein in the manufacture of a medicament for treating one or more disorders associated with IL-4/IL-13/p40 expression. In another aspect, provided is a method of one or more of detecting, diagnosing, or monitoring one or more of the disorders associate with IL-4/IL-13/p40 expression. For example, the anti-IL-4/IL-13/p40 antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent. With respect to all methods described herein, reference to anti-IL-4/IL-13/p40 antibodies also includes pharmaceutical compositions comprising the anti-IL-4/IL-13/p40 antibodies and one or more additional agents.

### Administration and Dosing

Typically, an antibody of the invention is administered in an amount effective to treat a condition as described herein. The antibodies the invention can be administered as an antibody per se, or alternatively, as a pharmaceutical composition containing the antibody.

The antibodies of the invention are administered by any suitable route in the form of a pharmaceutical composition adapted to such a route, and in a dose effective for the treatment intended.

In some embodiments, the antibodies may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In some aspects, IL-4/IL-13/IL-33 antibodies of the invention are administered subcutaneously. In some aspects, IL-4/IL-13/TSLP antibodies of the invention are administered subcutaneously. In some aspects, IL-4/IL-13/p40 antibodies of the invention are administered subcutaneously.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the antibodies of the invention or compositions containing said antibodies is based on a variety of factors, including the type, age, weight, sex and medical condition of the subject; the severity of the condition; the route of administration; and the activity of the particular antibody employed. Thus, the dosage regimen may vary widely. In one embodiment, the total daily dose of an antibody of the invention is typically from about 0.01 to about 100 mg/kg (i.e., mg antibody of the invention per kg body weight) for the treatment of the indicated conditions discussed herein. In another embodiment, total daily dose of the antibody of the invention is from about 0.1 to about 50 mg/kg, and in another embodiment, from about 0.5 to about 30 mg/kg.

### Co-Administration

The antibodies of the invention can be used alone, or in combination with one or more other therapeutic agents. The invention provides any of the uses, methods or compositions as defined herein wherein an antibody of the invention is used in combination with one or more other therapeutic agent discussed herein.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

Various formulations of the antibodies of the present invention (e.g., one or more of anti-IL-4, anti-IL-13, anti-IL-33, anti-TSLP, anti-IL-4/IL-13, anti-IL-4/IL-13/IL-33, anti-IL-4/IL-13/TSLP, anti-IL-4/IL-13/p40 antibodies) may be used for administration. In some embodiments, the antibodies may be administered neat. In some embodiments, the antibody and a pharmaceutically acceptable excipient may be in various formulations. Pharmaceutically acceptable excipients are known in the art and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 21st Ed. Mack Publishing, 2005.

In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

The antibodies (e.g., one or more of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies) as described herein can be administered using any suitable method, including by injection (e.g., intraperitoneally, intravenously, subcutaneously, intramuscularly, etc.). The antibody, e.g., monoclonal antibody or multispecific antibody, also be administered via inhalation, as described herein. Generally, for administration of the antibody of the present, the dosage depends upon the host treated and the particular mode of administration. In one embodiment, the dose range of the antibody of the present invention will be about 0.001 pg/kg body weight to about 20,000 pg/kg body weight. The term “body weight” is applicable when a patient is being treated. When isolated cells are being treated, “body weight” as used herein refers to a “total cell body weight”. The term “total body weight” may be used to apply to both isolated cell and patient treatment. All concentrations and treatment levels are expressed as “body weight” or simply “kg” in this application are also considered to cover the analogous “total cell body weight” and “total body weight” concentrations. However, those of ordinary skill in the art will recognize the utility of a variety of dosage range, for example, 0.01 μg/kg body weight to 20,000 μg/kg body weight, 0.02 μg/kg body weight to 15,000 μg/kg body weight, 0.03 μg/kg body weight to 10,000 μg/kg body weight, 0.04 μg/kg body weight to 5,000 μg/kg body weight, 0.05 μg/kg body weight to 2,500 μg/kg body weight, 0.06 μg/kg body weight to 1,000 μg/kg body weight, 0.07 μg/kg body weight to 500 μg/kg body weight, 0.08 μg/kg body weight to 400 μg/kg body weight, 0.09 μg/kg body weight to 200 μg/kg body weight or 0.1 μg/kg body weight to 100 μg/kg body weight. Further, those of skill will recognize that a variety of different dosage levels will be of use, for example, one or more selected from the group consisting of 0.0001 μg/kg, 0.0002 μg/kg, 0.0003 μg/kg, 0.0004 μg/kg, 0.005 μg/kg, 0.0007 μg/kg, 0.001 μg/kg, 0.1 μg/kg, 1.0 μg/kg, 1.5 μg/kg, 2.0 μg/kg, 5.0 μg/kg, 10.0 μg/kg, 15.0 μg/kg, 30.0 μg/kg, 50 μg/kg, 75 μg/kg, 80 μg/kg, 90 μg/kg, 100 μg/kg, 120 μg/kg, 140 μg/kg, 150 μg/kg, 160 μg/kg, 180 μg/kg, 200 μg/kg, 225 μg/kg, 250 μg/kg, 275 μg/kg, 300 μg/kg, 325 μg/kg, 350 μg/kg, 375 μg/kg, 400 μg/kg, 450 μg/kg, 500 μg/kg, 550 μg/kg, 600 μg/kg, 700 μg/kg, 750 μg/kg, 800 μg/kg, 900 μg/kg, 1 μg/kg, 5 μg/kg, 10 μg/kg, 12 μg/kg, 15 mg/kg, 20 mg/kg, and 30 mg/kg. All of these dosages are exemplary, and any dosage in-between these points is also expected to be of use in the invention. Any of the above dosage ranges or dosage levels may be employed for an antibody of the present invention. For repeated administrations over several days or longer, depending on the condition, the treatment is sustained until a desired suppression of symptoms occurs or until sufficient therapeutic levels are achieved.

Generally, for administration of antibodies provided herein, the candidate dosage can be administered daily, every week, every other week, every three weeks, every four weeks, every five weeks, every six weeks, every seven weeks, every eight weeks, every ten weeks, every twelve weeks, or more than every twelve weeks.

In some embodiments, the candidate dosage is administered daily with the dosage ranging from about any of 1 μg/kg, to 30 μg/kg, to 300 μg/kg, to 3 mg/kg, to 30 mg/kg, to 100 mg/kg or more, depending on the factors mentioned above. For example, daily dosage of about 0.01 mg/kg, about 0.03 mg/kg, about 0.1 mg/kg, about 0.3 mg/kg, about 1 mg/kg, about 2.5 mg/kg, about 3 mg/kg, about 5 mg/kg, about 10 mg/kg, about 15 mg/kg, and about 25 mg/kg may be used.

In some embodiments, the candidate dosage is administered every week with the dosage ranging from about any of 1 μg/kg, to 30 μg/kg, to 300 μg/kg, to 3 mg/kg, to 30 mg/kg, to 100 mg/kg or more, depending on the factors mentioned above. For example, a weekly dosage of about 0.01 mg/kg, about 0.03 mg/kg, about 0.1 mg/kg, about 0.3 mg/kg, about 0.5 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every two weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a bi-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, and about 30 mg/kg may be used.

In some embodiments, the candidate dosage is administered every three weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a tri-weekly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/k may be used.

In some embodiments, the candidate dosage is administered every month or every four weeks with the dosage ranging from about any of 1 μg/kg, to 30 μg/kg, to 300 μg/kg, to 3 mg/kg, to 30 mg/kg, to 100 mg/kg or more, depending on the factors mentioned above. For example, a monthly dosage of about 0.1 mg/kg, about 0.3 mg/kg, about 1 mg/kg, about 2.5 mg/kg, about 3 mg/kg, about 5 mg/kg, about 10 mg/kg, about 15 mg/kg, about 25 mg/kg, about 30 mg/kg, about 35 mg/kg, about 40 mg/kg, about 45 mg/kg, and about 50 mg/kg may be used.

In other embodiments, the candidate dosage is administered daily with the dosage ranging from about 0.01 mg to about 1200 mg or more, depending on the factors mentioned above. For example, daily dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, or about 1200 mg may be used.

In other embodiments, the candidate dosage is administered every week with the dosage ranging from about 0.01 mg to about 2000 mg or more, depending on the factors mentioned above. For example, weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other embodiments, the candidate dosage is administered every two weeks with the dosage ranging from about 0.01 mg to about 2000 mg or more, depending on the factors mentioned above. For example, bi-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg may be used.

In other embodiments, the candidate dosage is administered every three weeks with the dosage ranging from about 0.01 mg to about 2500 mg or more, depending on the factors mentioned above. For example, tri-weekly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or about 2500 mg may be used.

In other embodiments, the candidate dosage is administered every four weeks or month with the dosage ranging from about 0.01 mg to about 3000 mg or more, depending on the factors mentioned above. For example, monthly dosage of about 0.01 mg, about 0.1 mg, about 1 mg, about 10 mg, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500, about 2600 mg, about 2700 mg, about 2800 mg, about 2900 mg, or about 3000 mg may be used.

Other dosage regimens may also be useful, depending on the pattern of pharmacokinetic decay that the practitioner wishes to achieve. In one embodiment, the antibody of the present invention is administered in an initial priming dose followed by a higher and/or continuous, substantially constant dosage. In some embodiments, dosing from one to four times a week is contemplated. In other embodiments, dosing once a month or once every other month or every three months is contemplated. The progress of this therapy is easily monitored by conventional techniques and assays. The dosing regimen can vary over time.

For the purpose of the present invention, the appropriate dosage of an antibody (e.g., one or more selected from the group consisting of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies) will depend on the antibody or compositions thereof employed, the type and severity of symptoms to be treated, whether the agent is administered for therapeutic purposes, previous therapy, the patient's clinical history and response to the agent, the patient's clearance rate for the administered agent, and the discretion of the attending physician. Typically, the clinician will administer an antibody until a dosage is reached that achieves the desired result. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of symptoms. Alternatively, sustained continuous release formulations of antibodies may be appropriate. Various formulations and devices for achieving sustained release are known in the art.

In one embodiment, dosages for an antibody (e.g., one or more selected from the group consisting of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies) may be determined empirically in individuals who have been given one or more administration(s) of the antibody. Individuals are given incremental dosages of an antibody. To assess efficacy, an indicator of the disease can be followed.

In some embodiments, an antibody provided herein (e.g., one or more selected from the group consisting of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies) may be administered to a subject that has previously received one or more antibodies selected from the group consisting of anti-IL-4, anti-IL-13, anti-IL-33, anti-TSLP, or anti-p40 antibody therapeutic for treatment of a disease. In some embodiments, an antibody provided herein may be an administered to a subject that has previously received an antibody selected from the group consisting of anti-IL-4, anti-IL-13, anti-IL-33, anti-TSLP, or anti-p40 antibody therapeutic for treatment of a disease, and for which the previous anti-IL-4, anti-IL-13, anti-IL-33, anti-TSLP, or anti-p40 antibody therapeutic is of limited or no efficacy in the subject (e.g. for which the subject's disease is resistant to treatment with the prior anti-IL-4, anti-IL-13, anti-IL-33, anti-TSLP, or anti-p40 therapeutic).

Administration of an antibody in accordance with the method in the present invention can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced doses.

Therapeutic formulations of the antibody used in accordance with the present invention are prepared for storage by mixing an antibody having the desired degree of purity with optional pharmaceutically acceptable carriers, excipients or stabilizers (Remington, The Science and Practice of Pharmacy 21st Ed. Mack Publishing, 2005), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

### Kits

Another aspect of the invention provides kits comprising the antibody of the invention or pharmaceutical compositions comprising the antibody. A kit may include, in addition to the antibody of the invention or pharmaceutical composition thereof, diagnostic or therapeutic agents. A kit may also include instructions for use in a diagnostic or therapeutic method. In some embodiments, the kit includes the antibody or a pharmaceutical composition thereof and a diagnostic agent. In other embodiments, the kit includes the antibody or a pharmaceutical composition thereof and one or more therapeutic agents.

A further aspect of the invention is a kit comprising one or more selected from the group consisting of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies as disclosed herein above and instructions for use in accordance with any of the methods of the invention described herein. Generally, these instructions comprise a description of administration one or more selected from the group consisting of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies for the above described therapeutic treatments.

In yet another embodiment, the invention comprises kits that are suitable for use in performing the methods of treatment described herein. In one embodiment, the kit contains a first dosage form comprising one or more of the antibodies of the invention in quantities sufficient to carry out the methods of the invention. In another embodiment, the kit comprises one or more antibodies of the invention in quantities sufficient to carry out the methods of the invention and at least a first container for a first dosage and a second container for a second dosage.

Several aspects of the pharmaceutical compositions, prophylactic, or therapeutic agents of the invention are preferably tested in vitro, in a cell culture system, and in an animal model organism, such as a rodent animal model system, for the desired therapeutic activity prior to use in humans.

Toxicity and efficacy of the prophylactic and/or therapeutic protocols of the instant invention maybe determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it may be expressed as the ratio LD₅₀/ED₅₀. Prophylactic and/or therapeutic agents that exhibit large therapeutic indices are preferred.

Further, any assays known to those skilled in the art may be used to evaluate the prophylactic and/or therapeutic utility of the therapies or combinatorial therapies disclosed herein for treatment or prevention of cancer.

The instructions relating to the use of one or more selected from the group consisting of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies as described herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, ampules, tubes, bottles, jars, flexible packaging (e.g., sealed Mylar or plastic bags), and the like for each pharmaceutical composition and other included reagents, e.g., buffers, balanced salt solutions, etc., for use in administering the pharmaceutical compositions to subjects. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition selected from the group consisting of anti-IL-4, anti-IL-13, anti-IL-33, anti-TSLP, anti-IL-4/IL-13, anti-IL-4/IL-13/IL-33, anti-IL-4/IL-13/TSLP, and anti-IL-4/IL-13/p40 antibodies. The container may further comprise a second pharmaceutically active agent.

Normally, the kit comprises a container and a label or package insert(s) on or associated with the container.

Incorporated by reference herein for all purposes is the content of U.S. Provisional Patent Application Nos. 62/949,120 (filed Dec. 17, 2019) and 63/110,693 (Filed Nov. 6, 2020).

As used herein, “mammalian cells” include reference to cells derived from mammals including humans, rats, mice, hamsters, guinea pigs, chimpanzees, or macaques. The cells may be cultured in vivo or in vitro.

As used herein, the term “purified product” refers to a preparation of the product which has been isolated from the cellular constituents with which the product is normally associated or from other types of cells that may be present in the sample of interest.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The term “non-human animals” of the invention includes all non-human vertebrates, e.g., non-human mammals and non-mammals, such as non-human primates, sheep, dog, cow, chickens, amphibians, reptiles, mouse, rat, rabbit or goat etc., unless otherwise noted.

As used herein, the term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

As used herein, the terms “pharmaceutically acceptable excipient, carrier or adjuvant” or “acceptable pharmaceutical carrier” refer to an excipient, carrier or adjuvant that can be administered to a subject, together with at least one antibody of the present disclosure, and which does not destroy the activity of the antibody. The excipient, carrier or adjuvant should be nontoxic when administered with an antibody in doses sufficient to deliver a therapeutic effect.

As used herein, the term “ameliorating” means a lessening or improvement of one or more symptoms as compared to not administering an antibody molecule of the invention. “Ameliorating” also includes shortening or reduction in duration of a symptom.

As used herein, the terms “prevent”, “preventing” and “prevention” refer to the prevention of the recurrence or onset of one or more symptoms of a disorder in a subject as result of the administration of a prophylactic or therapeutic agent.

Potency is a measure of the activity of a therapeutic agent expressed in terms of the amount required to produce an effect of given intensity. A highly potent agent evokes a greater response at low concentrations compared to an agent of lower potency that evokes a smaller response at low concentrations. Potency is a function of affinity and efficacy. Efficacy refers to the ability of therapeutic agent to produce a biological response upon binding to a target ligand and the quantitative magnitude of this response.

### Biological Deposits

Representative materials of the present invention were deposited in the American Type Culture Collection, 10801 University Boulevard, Manassas, VA 20110-2209, USA, on Dec. 17, 2021.

Vector “DFab IL13-LC-IL4-HC” having ATCC Accession No. PTA-127192 comprises a DNA insert encoding the “DFab IL13-LC-IL4-HC” and comprises SEQ ID NO: 188. Vector “IL13-mFd” having ATCC Accession No. PTA-127193 comprises a DNA insert encoding the “IL13-mFd” and comprises SEQ ID NO: 187. Vector “IL4-Dfab LC” having ATCC Accession No. PTA-127194 comprises a DNA insert encoding the “IL4-Dfab LC” and comprises SEQ ID NO: 189. Vector “IL13-0001 VL” having ATCC Accession No. PTA-127195 comprises a DNA insert encoding the “IL13-0001 VL” and comprises SEQ ID NO: 199. Vector “IL13-0001 VH” having ATCC Accession No. PTA-127196 comprises a DNA insert encoding the “IL13-0001 VH” and comprises SEQ ID NO: 198. Vector “IL4-1040 VL” having ATCC Accession No. PTA-127197 comprises a DNA insert encoding the IL4-1040 VL” and comprises SEQ ID NO: 201. Vector “IL4-1040 VH” having ATCC Accession No. PTA-127198 comprises a DNA insert encoding the “IL4-1040 VH” and comprises SEQ ID NO: 200. Vector “TSLP-0875 VL” having ATCC Accession No. PTA-127199 comprises a DNA insert encoding the “TSLP-0875 VL” and comprises SEQ ID NO: 205. Vector “TSLP-0875 VH” having ATCC Accession No. PTA-127200 comprises a DNA insert encoding the “TSLP-0875 VH” and comprises SEQ ID NO: 204. Vector “SFab TSLP-LC” having ATCC Accession No. PTA-127201 comprises a DNA insert encoding the “SFab TSLP-LC” and comprises SEQ ID NO: 193. Vector “SFab TSLP-HC” having ATCC Accession No. PTA-127202 comprises a DNA insert encoding the “SFab TSLP-HC” and comprises SEQ ID NO: 192. Vector “SFab p40-LC” having ATCC Accession No. PTA-127203 comprises a DNA insert encoding the “SFab p40-LC” and comprises SEQ ID NO: 195. Vector “SFab p40-HC” having ATCC Accession No. PTA-127204 comprises a DNA insert encoding the “SFab p40-HC” and comprises SEQ ID NO: 194. Vector “p40-0003 VL” having ATCC Accession No. PTA-127205 comprises a DNA insert encoding the “p40-0003 VL” and comprises SEQ ID NO: 207. Vector “p40-0003 VH” having ATCC Accession No. PTA-127206 comprises a DNA insert encoding the “p40-0003 VH” and comprises SEQ ID NO: 206. Vector “SFab IL33-LC” having ATCC Accession No. PTA-127207 comprises a DNA insert encoding the “SFab IL33-LC” and comprises SEQ ID NO: 191. Vector “SFab IL33-HC” having ATCC Accession No. PTA-127208 comprises a DNA insert encoding the “SFab IL33-HC” and comprises SEQ ID NO: 190. Vector “IL33-0726 VL” having ATCC Accession No. PTA-127209 comprises a DNA insert encoding the “IL33-0726 VL” and comprises SEQ ID NO: 203. Vector “IL33-0726 VH” having ATCC Accession No. PTA-127210 comprises a DNA insert encoding the “IL33-0726 VH” and comprises SEQ ID NO: 202.

Vector “TSLP-0855 VL” having ATCC Accession No. PTA-______ comprises a DNA insert encoding the “TSLP-0855 VL” and comprises SEQ ID NO: 217. Vector “TSLP-0855 LC” having ATCC Accession No. PTA-______ comprises a DNA insert encoding the “TSLP-0855 LC” and comprises SEQ ID NO: 219. Vector “TSLP-0871 VL” having ATCC Accession No. PTA-______ comprises a DNA insert encoding the “TSLP-0871 VL” and comprises SEQ ID NO: 218. Vector “TSLP-0871 LC” having ATCC Accession No. PTA-______ comprises a DNA insert encoding the “TSLP-0871 LC” and comprises SEQ ID NO: 220.

| | | ATCC Accession | SEQ ID |
|---|---|---|---|
| Antibody | Description | No. | NO: |
| IL413TSLP-1024 | DFab IL13-LC-IL4-HC | PTA-127192 | 188 |
| IL413P40-0705 | IL13-mFd | PTA-127193 | 187 |
| IL13433-1258 | IL4-Dfab LC | PTA-127194 | 189 |
| | IL13-0001 VL | PTA-127195 | 199 |
| | IL13-0001 VH | PTA-127196 | 198 |
| | IL4-1040 VL | PTA- 127197 | 201 |
| | IL4-1040 VH | PTA-127198 | 200 |
| IL413TSLP-1024 | TSLP-0875 VL | PTA-127199 | 205 |
| IL413TSLP-1024 | TSLP-0875 VH | PTA-127200 | 204 |
| IL413TSLP-1024 | SFab TSLP-LC | PTA-127201 | 193 |
| IL413TSLP-1024 | SFab TSLP-HC | PTA-127202 | 192 |
| IL413P40-0705 | SFab p40-LC | PTA-127203 | 195 |
| IL413P40-0705 | SFab p40-HC | PTA-127204 | 194 |
| IL413P40-0705 | p40-0003 VL | PTA-127205 | 207 |
| IL413P40-0705 | p40-0003 VH | PTA-127206 | 206 |
| IL13433-1258 | SFab IL33-LC | PTA-127207 | 191 |
| IL13433-1258 | SFab IL33-HC | PTA-127208 | 190 |
| IL13433-1258 | IL33-0726 VL | PTA-127209 | 203 |
| IL13433-1258 | IL33-0726 VH | PTA-127210 | 202 |
| IL413TSLP-1028 | TSLP-0855 VL | PTA- | 217 |
| IL413TSLP-1028 | TSLP-0855 LC | PTA- | 219 |
| IL413TSLP-1037 | TSPL-0871 VL | PTA- | 218 |
| IL413TSLP-1037 | TSPL-0871 LC | PTA- | 220 |

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions; the materials will be promptly replaced on notification with another of the same. Availability of the deposited material is not to be construed as a license to practice the invention in contravention of the rights granted under the authority of any government in accordance with its patent laws.

### Materials and Methods

Various techniques for the production of antibodies have been described which include the traditional hybridoma method for making monoclonal antibodies, recombinant techniques for making antibodies (including chimeric antibodies, e.g., humanized antibodies), antibody production in transgenic animals and the recently described phage display technology for preparing “fully human” antibodies.

Provided herein are methods of making any of the antibodies provided herein. The antibodies of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

Any suitable method for preparing multispecific antibodies may be used to prepare multispecific antibodies provided herein (e.g. depending on the choice of antibody features and components).

According to one approach to making multispecific antibodies, antibody variable domains with the desired binding specificities are fused to immunoglobulin constant region sequences. The fusion preferably is with an immunoglobulin heavy chain constant region, comprising at least part of the hinge, CH2 and CH3 regions. In some embodiments, the first heavy chain constant region (CH1), containing the site for light chain binding can be present in at least one of the fusions. In some embodiments, polynucleotides encoding the immunoglobulin heavy chain fusions and, if desired, the immunoglobulin light chain, may be inserted into separate expression vectors, and may be cotransfected into a suitable host organism. In other embodiments the coding sequences for two or all three polypeptide chains may be inserted into one expression vector when the expression of at least two polypeptide chains in equal ratios results in high yields or when the ratios are of no particular significance.

In one approach, the multispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the multispecific molecule, facilitates the separation of the desired multispecific compound from unwanted immunoglobulin chain combinations. This approach is described in PCT Publication No. WO 94/04690.

In another approach, the multispecific antibodies are composed of amino acid modification in the first hinge region in one arm, and the substituted amino acid in the first hinge region has an opposite charge to the corresponding amino acid in the second hinge region in another arm. This approach is described in International Patent Application No. PCT/US2011/036419 (WO2011/143545).

In another approach, the formation of a desired heteromultimeric or heterodimeric protein (e.g., bispecific antibody) is enhanced by altering or engineering an interface between a first and a second Fc chain. In this approach, the multispecific antibodies may be composed of a CH3 region, wherein the CH3 region comprises a first CH3 polypeptide and a second CH3 polypeptide which interact together to form a CH3 interface, wherein one or more amino acids within the CH3 interface destabilize homodimer formation and are not electrostatically unfavorable to homodimer formation. This approach is described in International Patent Application No. PCT/US2011/036419 (WO2011/143545). In some embodiments, one Fc chain of a bispecific antibody can comprise amino acid modifications at positions 223 and 228 (e.g., (C223E or C223R), and (P228E or P228R)) in the hinge region and at position 409 (e.g., K409R (EU numbering scheme)) in the CH3 region of human IgG2, and the other Fc chain of the bispecific antibody can comprise amino acid modifications at positions 223, 225 and 228 (e.g., (C223E or C223R), (E225R), and (P228E or P228R)) in the hinge region and at position 368 (e.g., L368E (EU numbering scheme)) in the CH3 region of human IgG2. In other embodiments, one Fc chain of a bispecific antibody can comprise amino acid modifications at positions 223 and 228 (e.g., (C223E or C223R) and (P228E or P228R)) in the hinge region and at position 368 (e.g., L368E (EU numbering scheme)) in the CH3 region of human IgG2, and the other Fc chain of the bispecific antibody can comprise amino acid modifications at positions 223, 225 and 228 (e.g., (C223E or C223R), (E225R), and (P228E or P228R)) in the hinge region and at position 409 (e.g., K409R (EU numbering scheme)) in the CH3 region of human IgG2. In some embodiments, a bispecific antibody can comprise amino acid modifications at positions 221 and 228 (e.g., (D221R or D221E) and (P228R or P228E)) in the hinge region and at position 409 or 368 (e.g., K409R or L368E (EU numbering scheme)) in the CH3 region of human IgG1. In some embodiments, a bispecific antibody can comprise amino acid modifications at positions 228 (e.g., (P228E or P228R)) in the hinge region and at position 409 or 368 (e.g., R409 or L368E (EU numbering scheme)) in the CH3 region of human IgG4.

In some embodiments, a multispecific antibody may have knob-in-hole mutations in the Fc chains. For example, in some embodiments, in a bispecific antibody having knob-in-hole mutations, the first Fc chain of the antibody Fc domain has one or more mutations to form a “knob”, and the second Fc chain of the antibody Fc domain has one or more mutations to form a “hole” (or vice-versa). Exemplary knob-in-hole engineering of antibodies is described in U.S. Pat. No. 5,731,168, PCT Publication No. WO2009089004, U.S. Publication No. 20090182127, Marvin and Zhu, Acta Pharmacologica Sincia (2005) 26(6):649-658 and Kontermann (2005) Acta Pharacol. Sin., 26:1-9.

A “knob” refers to at least one amino acid side chain which projects from the interface of a first polypeptide (e.g. first Fc chain) and is therefore positionable in a compensatory hole in an adjacent second polypeptide (e.g. second Fc chain) so as to stabilize a heterodimer, and thereby favor heterodimer formation over homodimer formation. The knob may exist in the original interface or may be introduced synthetically (e.g., by altering a nucleic acid encoding the interface). Normally, nucleic acid encoding the interface of the first polypeptide is altered to encode the knob. To achieve this, the nucleic acid encoding at least one original amino acid residue in the first polypeptide is replaced with nucleic acid encoding at least one “import” amino acid residue which has a larger side chain volume than the original amino acid residue. Certain import residues for the formation of a knob are generally naturally occurring amino acid residues and are preferably selected from arginine (R), phenylalanine (F), tyrosine (Y) and tryptophan (W).

A “hole” refers to at least one amino acid side chain which is recessed from the interface of a second polypeptide (e.g. second Fc chain) and therefore accommodates a corresponding knob in an adjacent first polypeptide (e.g. first Fc chain). The hole may exist in the original interface or may be introduced synthetically (e.g., by altering a nucleic acid encoding the interface). Normally, nucleic acid encoding the interface of the second polypeptide is altered to encode the hole. To achieve this, the nucleic acid encoding at least one original amino acid residue of the second polypeptide is replaced with DNA encoding at least one “import” amino acid residue which has a smaller side chain volume than the original amino acid residue. Certain import residues for the formation of a hole are usually naturally occurring amino acid residues and are preferably selected from alanine (A), serine (S), threonine (T) and valine (V).

Exemplary knob-in-hole (KiH) CH3 domain pairs include: SEQ ID NO: 105 and SEQ ID NO: 111; SEQ ID NO: 106 and SEQ ID NO: 111; SEQ ID NO: 106 and SEQ ID NO: 112; SEQ ID NO: 114 and SEQ ID NO: 117; and SEQ ID NO: 139 and SEQ ID NO: 141.

The term “interface,” as used herein typically refers to any amino acid residue present in the domain that can be involved in first polypeptide and second polypeptide contacts. An “original amino acid” residue is one which is replaced by an “import amino acid” residue which can have a smaller or larger side chain volume than the original residue. The import amino acid residue can be a naturally occurring or non-naturally occurring amino acid residue, but preferably is the former. “Naturally occurring” amino acid residues are those residues encoded by the genetic code. By “non-naturally occurring” amino acid residue is meant a residue which is not encoded by the genetic code, but which is able to covalently bind adjacent amino acid residue(s) in the polypeptide chain. Examples of non-naturally occurring amino acid residues are norleucine, ornithine, norvaline, homoserine and other amino acid residue analogues such as those described in Ellman et al., Meth. Enzym. 202:301-336 (1991).

Once a nucleic acid sequence encoding molecules of the invention (i.e., binding domains) has been obtained, the vector for the production of the molecules may be produced by recombinant DNA technology using techniques well known in the art.

The polynucleotides encoding the antibody (binding domains of the present invention may include an expression control polynucleotide sequence operably linked to the antibody coding sequences, including naturally-associated or heterologous promoter regions known in the art. The expression control sequences may be eukaryotic promoter systems in vectors capable of transforming or transfecting eukaryotic host cells, but control sequences for prokaryotic hosts may also be used. Once the vector has been incorporated into the appropriate host cell line, the host cell is propagated under conditions suitable for expressing the nucleotide sequences, and, as desired, for the collection and purification of the antibodies. Eukaryotic cell lines include the CHO cell lines, various COS cell lines, HeLa cells, myeloma cell lines, transformed B-cells, or human embryonic kidney cell lines.

In one embodiment, the DNA encoding the antibodies of the invention is isolated and sequenced using conventional procedures (e.g., by using oligonucleotide probes that are capable of binding specifically to genes encoding the heavy and light chains of antibodies). Once isolated, the DNA may be placed into expression vectors, which are then transfected into host cells such as simian COS cells, CHO cells, or myeloma cells that do not otherwise produce immunoglobulin protein, to obtain the synthesis of monoclonal antibodies in the recombinant host cells. The DNA also may be modified, for example, to improve one or more properties of the corresponding antibody (e.g. binding affinity, immunogenicity, etc.).

In one aspect, the invention provides a method of making any of the polynucleotides described herein. For example, the polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art (e.g., Sambrook et al., 1989).

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, CoIE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cells capable of over-expressing heterologous DNAs can be used for the purpose expressing genes encoding the antibody, polypeptide or protein of interest. Non-limiting examples of mammalian host cells include but not limited to COS, HeLa, and CHO cells. See also PCT Publication No. WO87/04462. Suitable non-mammalian host cells include prokaryotes (such as E. coli or B. subtillis) and yeast (such as S. cerevisae, S. pombe; or K. lactis). A cell overexpressing the antibody or protein of interest can be identified by known screening methods.

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

In some embodiments of the invention, the antibody comprises a modified constant region, such as a constant region that has increased affinity to a human Fc gamma receptor, is immunologically inert or partially inert, e.g., does not trigger complement mediated lysis, does not stimulate antibody-dependent cell mediated cytotoxicity (ADCC), or does not activate macrophages; or has reduced activities (compared to the unmodified antibody) in any one or more of the following: triggering complement mediated lysis, stimulating antibody-dependent cell mediated cytotoxicity (ADCC), or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 29:2613-2624, 1999; PCT Application No. PCT/GB99/01441; and/or UK Application No. 9809951.8. In still other embodiments, the constant region is aglycosylated for N-linked glycosylation. In some embodiments, the constant region is aglycosylated for N-linked glycosylation by mutating the glycosylated amino acid residue or flanking residues that are part of the N-glycosylation recognition sequence in the constant region. For example, N-glycosylation site N297 may be mutated to A, Q, K, or H. See, Tao et al., J. Immunology 143: 2595-2601, 1989; and Jefferis et al., Immunological Reviews 163:59-76, 1998. In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications include antibodies that have been modified as described in PCT Publication No. WO99/58572. These antibodies comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain is capable of specifically binding either or both of the FcRn or the FcγRIIb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy.

In some embodiments, the Fc chain of an antibody provided herein may be modified to ablate effector function. For example, the Fc chain of human IgG1 may be modified to introduce mutations L234A, L235A and G237A using standard primer-directed PCR mutagenesis to oblate effector function due to binding to FcγRIII, providing for an effector function null phenotype (Canfield et al., J. Exp. Med (1991) 173: 1483-1491; Shields et al., J. Biol. Chem. (2001) 276:6591-604).

In some embodiments, a multispecific antibody provided herein may be engineered to comprise at least one cysteine residue that may interact with a counterpart cysteine residue on another polypeptide chain of the invention to form an inter-chain disulfide bond. The inter-chain disulfide bonds may serve to stabilize the multispecific antibody, improving expression and recovery in recombinant systems, resulting in a stable and consistent formulation, as well as, improving the stability of the isolated and/or purified product in vivo. The cysteine residue or residues may be introduced as a single amino acid or as part of larger amino-acid sequence, e.g., hinge region, in any portion of the polypeptide chain. In a specific aspect, at least one cysteine residue is engineered to occur at the C-terminus of the polypeptide chain.

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### EXAMPLES

The following examples of specific aspects for carrying out the present invention are offered for illustrative purposes only and are not intended to limit the scope of the present invention in any way.

### Example 1 Pfabat Numbering Method Developed for Consistent Antibody Numbering

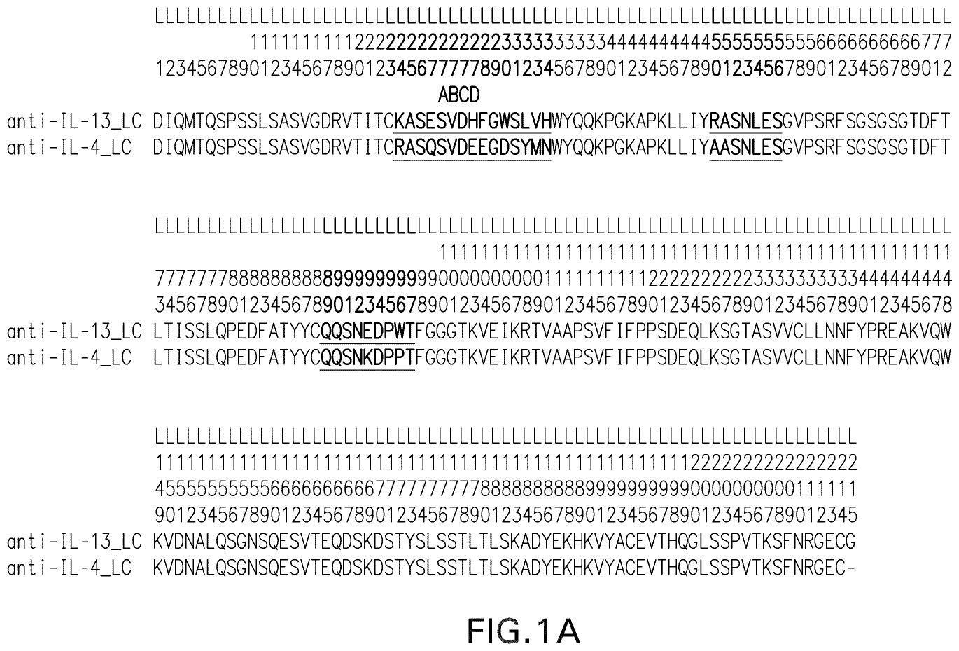

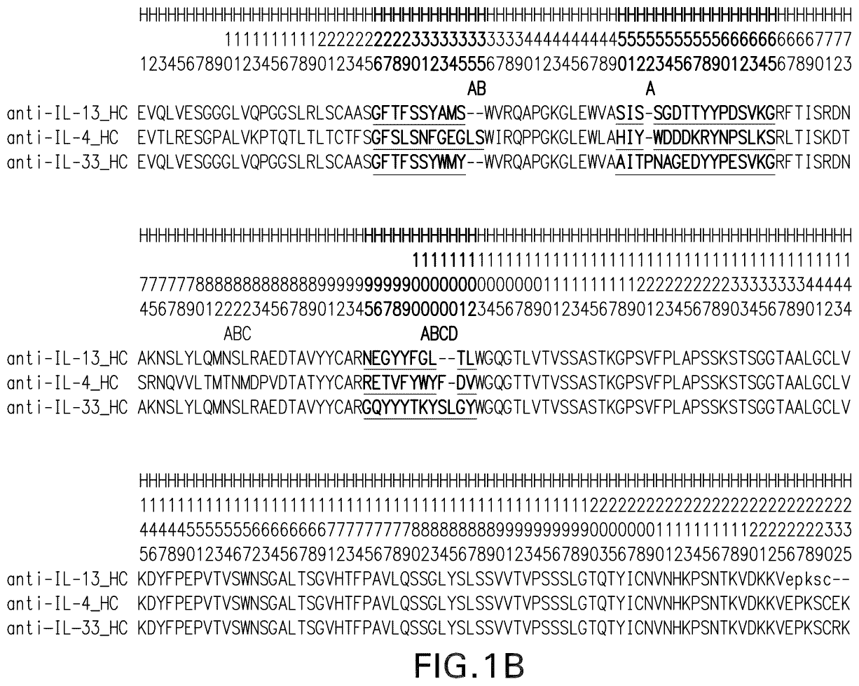

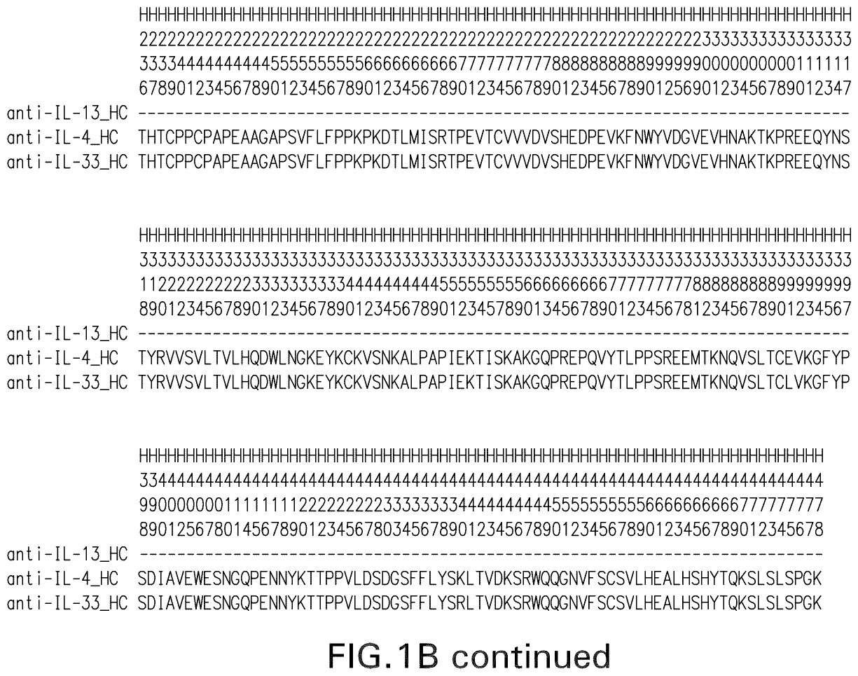

The Pfabat numbering method is a defined algorithm for consistent antibody numbering, based on the Kabat numbering system (Sequences of Proteins of Immunological Interest, Fifth Edition by Kabat et al., NIH Publication NO: 91-3242, 1991). Unlike many other computational implementations of Kabat numbering, Pfabat numbers entire human IgG1 heavy and light chains, including the constant (C) regions and heavy chain hinge. FIG. 1 shows example numbering, with complementarity-determining regions (CDR) residues underlined in bold. For the light chain, the CDR definitions are: CDRL1 from residues L24-L34; CDRL2 from residues L50-L56; CDRL3 from residue L89-L97; CDRH1 from residues H26-H35 (including insertion positions such as H35B); CDRH2 from residues H50-H65; and CDRH3 from residues H95-H102. Note that the definition of CDRH1 used herein includes positions H26-H29, which are not included in some other interpretations of Kabat numbering.

The Pfabat algorithm is not designed to number the Glycine-Serine Linker (GGGGS: SEQ ID NO: 104) joining the carboxy terminus of the anti-IL-13 light chain (Cys L214) to the amino terminus of the anti-IL-4 heavy chain (Glu H1), although first Gly of the linker is numbered as part of the light chain constant region (Gly L215).

### Example 2 Generation of IL-4, IL-13, TSLP, IL-33, IL-12 and IL-23 Recombinant Proteins

Complementary DNA (cDNA) fragments encoding full-length human IL-13 (AAK53823) and cynomolgus monkey IL-13 (ABG75889) were fused to a FLAG affinity purification tag at the N-terminus within a prokaryotic expression vector, expressed in BL21 DE3 E. coli host cells and protein was purified from bacterial inclusion bodies. Mouse IL-13 and rat IL-13 were purchased from R&D Systems (Minneapolis, MN), catalog numbers 413-ML and 1945-RL, respectively.

Human IL-4 cytokine (P05112) was generated at Syngene. Cynomolgus monkey IL-4 cytokine (P79339) was purchased from Kingfisher Biotech (catalog #RP1184Y-025). Mouse, rat and rabbit IL-4 were purchased from R&D Systems (Minneapolis, MN), catalog numbers 404-ML, 504-RL and 6939-RB, respectively.

Complementary DNA (cDNA) fragments encoding full-length (long-form or If) human thymic stromal lymphopoietin (IfhTSLP, NP_149024), cynomolgus monkey TSLP (cynoTSLP, XP_005557555), mouse TSLP (mTSLP, NP_067342), rat TSLP (ratTSLP, XP_008770274), rabbit TSLP (RabTSLP, G1TYN9) were cloned into mammalian expression vectors. The N-terminus of TSLP was fused to the CH2 and CH3 domains of human IgG1 with an intervening TEV (tobacco etch virus) protease recognition site. Mutations were introduced in CH3 domain to disrupt molecule dimerization (1). The C-terminus of TSLP was fused to an Avi tag (site-specific biotinylation), a V5 tag and a poly-His tag (CH23LS-TSLP-avi-v5-his10). cDNA was transfected into Expi293F™ cells according to manufacturer's protocol (Thermo Fisher, Grand Island, NY, USA). To generate biotinylated antigens, cDNAs encoding TSLP and E. coli biotin ligase BirA (2) were co-transfected into Expi293F™ cells. Antigens were isolated and purified using MabSelect™ SuRe™ LX resin (GE Life Sciences) and/or Ni Superflow resin (Qiagen) followed by preparative Superdex 200 pre-packed size exclusion chromatography column (GE Life Sciences). In the case of cleaved full-length human and cyno TSLP generation (IfTSLP-avi-v5-his10), the purified proteins were cleaved by AcTEV protease (Invitrogen™) at 30° C. for 72 hours. The cleavage reaction was passed through a MabSelect™ SuRe™ LX (GE Life Sciences) column to remove the CH23 fragment and the flow-through was collected and concentrated. The flow-through with cleaved TSLP was further purified using a preparative Superdex 200 column (GE Life Sciences).

Recombinant human and cynomolgus monkey IL-12 (p35+p40) and IL-23 (p19+p40) were generated using amino acid sequences derived from the accession numbers listed below and the corresponding nucleic acid sequences were transiently expressed into Expi293F™ cells (Table 1). Briefly, cDNA fragments encoding the cytokines were cloned into mammalian expression vectors and transiently transfected into Expi293F™ (Thermo Fisher) cells. Secreted antigens were isolated and purified using an affinity-based column followed by size exclusion chromatography column, similar to methodology described above. Purity of the antigens post-purification were confirmed using analytical size exclusion chromatography and SDS-PAGE analysis. Mouse IL-12 and rat IL-12 were purchased from R&D Systems (Minneapolis, MN), catalog numbers 419-ML and 1760-RL, respectively. Mouse IL-23 and rat IL-23 were purchased from R&D Systems (Minneapolis, MN), catalog numbers 1887-ML and 3136-RL, respectively.

TABLE 1
Accession Numbers for Human and
Cynomolgus p35 and p40 Cytokines.

| | Accession |
|---|---|
| Name | Number |
| Human IL12A p35 | NP_000873 |
| Human IL12B p40 | NP_002178 |
| Human IL23A p19 | NP_057668 |
| Cyno IL12A p35 | XP_005546300 |
| Cyno IL12B p40 | NP_001274204 |
| Cyno IL23A p19 | NP_001274588 |

Recombinant human IL-33 amino acid sequence Ser112-Thr270 (accession #095760) prepared in E. coli was purchased from R&D Systems (Minneapolis, MN, catalog #3625-IL, SEQ ID. NO. 539). In order to eliminate oxidation-based inactivation of IL-33 (Cohen et al., 2015), IL-33 (mm2), a variant of human IL-33 in which all four cysteine residues were changed to serine residues, was produced. Escherichia coli (E. coli) cells expressing IL-33 (mm2) were induced with Isopropyl p-D-1-thiogalactopyranoside (IPTG), harvested, and lysed by high shear homogenization (Microfluidizer MV1, Microfluidics, Westwood MA). The cytosolic fraction was centrifuged, batch-bound to TALON resin (Clontech, Mountain View, CA), washed in 10 mM imidazole in phosphate-buffered saline (PBS), and eluted in 200 mM imidazole in PBS. Pooled fractions were concentrated and further purified by size-exclusion chromatography on a Superdex 75 16/60 column (GE Healthcare Life Sciences, Pittsburgh, PA) in PBS. Cynomolgus monkey IL-33 WT (Pfizer, WRS-072216) was provided in PBS containing 1 mM DTT.

### Derivation of Anti-IL-4 Fab Binding Domain

### Example 3

### Derivation of Humanized Anti-IL-4 Antibody IL4-1285 with Increased Transient Expression Titer

Humanized anti-IL-4 heavy and light chain variable region amino acid sequences (SEQ ID NO: 5 and SEQ ID NO: 15, respectively) obtained from Holmes et al. U.S. Pat. No. 5,928,904 SmithKline Beecham Corporation Jul. 27, 1999 (SEQ ID NO: 12 and SEQ ID NO: 14, Holmes et al.) were joined to human IgG1 harboring mutations L(247)A, L(248)A and G(250)A (Pfabat numbering) to minimize effector function and human Kappa constant region, to generate IL4-1284 heavy chain (SEQ ID NO: 10) and IL4-1284 light chain (SEQ ID NO: 17), respectively. DNA encoding anti-IL-4 IL4-1284 antibody was transiently transfected into COS-1 cells to generate protein and the resultant conditioned medium containing the IL4-1284 was quantitated using a total human IgG ELISA. IL4-1284 antibody exhibited a sub-optimal expression level, specifically <10 mg/liter/48 hours (Table 2), so further engineering was required to improve biophysical properties. An alternative humanized anti-IL-4 V_{L} variant was constructed by grafting IL4-1284 V_{L} CDR regions onto the IGKV1-39*01 (DPK9) human germline framework. Precisely, the CDRs as defined by Pfabat (SEQ ID NO: 11, SEQ ID NO: 12 and SEQ ID NO: 13) were grafted onto the IGKV1-39*01 human germline acceptor framework with a JK4 segment (SEQ ID NO: 14) and this amino acid sequence is set forth as SEQ ID NO: 21 IL4-1286 V_{L} (hu3B9 V_{L} v2.6 V_{L}). An additional humanized V_{L} variant based on IL4-1284 antibody was engineered to restore a Threonine (T) residue at L97 (Pfabat numbering) present in the parental mouse CDRL3 that was mutated to an Arginine (R) in the humanized IL4-1284 variant IL4-1285 V_{L} (hu3B9 VL v2.0). The IL4-1285 V_{L} and IL4-1286 V_{L} were then fused to the human Kappa constant region (SEQ ID NO: 16) within a proprietary expression vector to generate IL4-1285 LC and IL4-1286 LC. Both the IL4-1285 and IL4-1286 light chains were individually combined with the IL4-1284 heavy chain (SEQ ID NO: 10) to generate antibody IL4-1285 and IL4-1286 antibodies, respectively. African Green Monkey Kidney cells (COS-1) were transiently transfected with DNA encoding IL4-1285 and IL4-1286 antibodies to generate protein and the resultant conditioned medium containing the antibodies was quantitated using a total human IgG sandwich ELISA. IL-4 bioactivity was determined using the pSTAT6 phosphorylation assay. IL-4 binding to Type I (IL-4Rα/γ common), or IL-4 or IL-13 binding to type II (IL-4Rα/IL-13Rα1) receptor triggers the phosphorylation and nuclear translocation of the transcription factor, STAT6. For this assay, IL-4Rα/IL-13Rα1-expressing HT-29 human colonic epithelial cells (American Type Culture Collection, Manassas, VA) were grown as an adherent monolayer and then dislodged from the flask using trypsin, washed into fresh medium and recombinant human IL-4 or IL-13 was added at varying concentrations. For assays testing antibody inhibition of cytokine responses, recombinant human IL-4 (0.1-0.5 ng/ml; R&D Systems) was added along with dilutions of antibodies ranging from 500 to 0.4 ng/mL. Cells were incubated at 37° C. for 30 minutes and then washed with ice-cold phosphate-buffered saline (PBS) containing 1% bovine-serum albumin (BSA). Cells were fixed by incubating with 1% paraformaldehyde in PBS for 15 minutes at 37° C. and then washed with PBS containing 1% BSA. To permeabilize the nucleus, cells were incubated overnight at −20° C. in absolute methanol, washed with PBS containing 1% BSA and then stained with AlexaFluor 488-labeled antibody to STAT6 (pY641; BD Biosciences, San Diego, CA). Fluorescence was analyzed by flow cytometry (BD Biosciences) and analyzed using FlowJo software version 7.2.4 (Tree Star Inc, Ashland, OR). Values for EC50 and IC₅₀ were calculated from cytokine and antibody dose titration data using GraphPad PRISM version 5.02 (GraphPad Software Inc, La Jolla, CA).

IL4-1285 (hu3B9 VL v2.0) antibody showed a 6-fold improvement of expression level relative to the IL4-1284 (hu3B9) antibody, as well as a minor 1.7-fold improvement in IL-4-induced STAT6 phosphorylation bioactivity in HT-29 cells (Table 2 and method described in IL13433-1258 Biology Example 1.1). Additionally, presence of Threonine versus an Arginine residue at L97 in CDRL3 within the IGKV1-39*01 human germline CDR grafted V_{L} significantly improved neutralization of IL-4 induced phosphorylation and nuclear translocation of the pSTAT6 transcription factor and marginally increased the transient expression level (IL4-1285 versus IL4-1286, Table 2).

TABLE 2
Transient COS-1 Expression Titers and IL-4-
induced pSTAT6 Phosphorylation Bioactivity
for the Humanized Anti-IL-4 Antibody Variants

| Antibody | | Expression Titer | pSTAT6 |
|---|---|---|---|
| Variant | Variant Aliases | (mg/L/48 hours) | IC₅₀ [pM] |
| IL4-1284 | IL13433-1284, hu3B9 | 4.0 | 125.0 |
| IL4-1285 | IL13433-1285, hu3B9 | 24.7 | 71.5 |
| | VLv2.0 | | |
| IL4-1286 | IL13433-1286, hu3B9 | 15.0 | 750.0 |
| | VLv2.6 | | |

### Example 4

### Engineering IL4-1285 Antibody to Increase Binding Affinity and Potency of Neutralization for the IL-4 Cytokine

Humanized IL4-1285 antibody underwent engineering efforts to increase IL-4 binding affinity to improve potency of IL-4 cytokine neutralization. To facilitate phage display, the variable domain regions of IL4-1285 antibody were cloned in a single-chain variable fragment (scFv) format using a VL-VH orientation with a (G4S)3 flexible linker within the phage-display vector pWRIL-1. The anti-IL-4 IL4-1285 scFv with IL-4 co-crystal structure (EXAMPLE 13) was determined and used to guide the affinity maturation process. The IL4-1285 V_{H} domain was optimized by phage display using a library built on soft mutagenesis and homolog scanning mutagenesis, targeting amino acids in CDRs at positions chosen on the basis of their known potential to make contact with the antigen, or to affect CDR structure. A two-round selection campaign was employed for each of the sub libraries, involving an extremely stringent initial first round of selection against 100 pM biotinylated human IL-4 with an incorporated overnight ‘off-rate’ challenge with a 1000-fold excess (100 nM) of human IL-4. In the second round of selection, 50 pM biotinylated human IL-4 was used without any ‘off-rate’ challenge. A panel of clones was randomly picked from each round of phage selection outputs for the anti-IL-4 selections. Analysis of these selected clones was performed via induction of scFv expression and the use of E. coli periplasmic preparations (peripreps) in a homogeneous time resolved fluorescence (HTRF) competition assay. Unique clones which showed increased competition in comparison to the parental IL4-1285 scFv were identified and taken forward for further analysis. These clones were reformatted to full-length IgG1-effector function- reduced/human Kappa antibodies and proteins were produced transiently in HEK-293 cells for further evaluation.

Variants selected based on improved IL-4 binding activities in the HTRF competition assay were monitored by bioassay for increased bioactivity. Because it had the greatest sensitivity range, the CD23 expression assay with primary human monocytes was primarily used for this analysis. Specifically, mononuclear cells were isolated from human peripheral blood by layering over Histopaque (Sigma Aldrich). Cells were washed into RPMI medium containing 10% heat-inactivated Fetal Calf Serum (FCS), 50 U/mL penicillin, 50 μg/mL streptomycin, 2 mM L-glutamine, and plated in a 48-well tissue culture plate (Costar/Corning). Recombinant human IL-4 was added at dilutions ranging from 100 to 0.01 ng/mL. For assays testing antibody inhibition of cytokine responses, 0.5 ng/mL human IL-4 was added along with dilutions of the antibody variants ranging from 500 to 0.4 ng/mL. Cells were incubated overnight at 37° C. in a 5% CO₂ incubator. The next day, cells were harvested from wells using non-enzymatic Cell Dissociation Solution (Sigma Aldrich), and then washed into ice-cold PBS containing 1% BSA. Cells were incubated with phycoerythrin (PE)-labeled antibody to human CD23 (BD Biosciences), and Cy-Chrome-labeled antibody to human CD11b (BD Biosciences). Monocytes were gated based on high forward and side light scatter, and expression of CD11b. CD23 expression on monocytes was determined by flow cytometry using a flow cytometer (BD Biosciences), and the percentage of CD23-positive cells was analyzed with CellQuest software (BD Biosciences). Because the CD23 expression assay is run with human peripheral blood, the monocyte CD23 expression assay shows subtle variations in response based on donor. Therefore, the responses of the affinity optimized variants were compared to the IL4-1285 parental control, respectively, run in each individual assay. Data are expressed as % maximal response, which typically ranged from 65-85% CD23+ monocytes. Neutralization of IL-4-induced STAT6 phosphorylation bioactivity in HT-29 cells was also measured for select variants as a comparator to parental anti-IL-4 variants.

Several anti-IL-4 variants were identified that exhibit a significant increase in potency of IL-4 neutralization in the CD23 expression bioassay in the range of 10-48-fold improvement and a modest improvement in neutralizing IL-4 induced pSTAT6 phosphorylation bioactivity in HT-29 cells since this assay window is narrow (Table 3). Variants of IL4-1285 with a modest or 5-10-fold increase in potency of IL-4 neutralization in the CD23 expression bioassay were also obtained using this phage display approach.

TABLE 3
Select Affinity Matured Anti-IL-4 Antibody Variants Exhibit a Significant Increase in
Potency of IL-4 Neutralization in the CD23 Expression Bioassay and Modest Improvement
in pSTAT6 Bioactivity

| | | Neutralization of IL-4-induced | | | Neutralization of IL-4 | |
|---|---|---|---|---|---|---|
| | | pSTAT6 Phosphorylation | | | Induced CD23 Expression | |
| | | | Fold | Fold | | Fold |
| Anti-IL-4 | | | bioactivity | bioactivity | | bioactivity |
| Antibody | Variant | IC₅₀ | relative to | relative to | IC₅₀ | relative to |
| Variant | Aliases | [pM] | IL4-1284 | IL4-1285 | [pM] | IL4-1285 |
| IL4-1284 | IL13433- | 125.0 | 1 | 0.6 | ND | ND |
| | 1284, hu3B9 | | | | | |
| IL4-1285 | IL13433- | 71.5 | 1.8 | 1 | 96.2 | 1 |
| | 1285, hu3B9 | | | | | |
| | VL v2.0 | | | | | |
| IL4-1286 § | IL13433- | 750.0 | 0.2 | 0.1 | ND | ND |
| | 1286, hu3B9 | | | | | |
| | VL v2.6 | | | | | |
| IL4-1287 † | IL13433- | 82.0 | 1.5 | 0.9 | 5.6 | 17.2 |
| | 1287, | | | | | |
| | hu3B9_A01- | | | | | |
| | CDRH1 | | | | | |
| IL4-1288 † | IL13433- | 56.0 | 2.2 | 1.3 | 5.0 | 19.2 |
| | 1288, | | | | | |
| | hu3B9_A02- | | | | | |
| | CDRH1 | | | | | |
| IL4-1289 † | IL13433- | 20.0 | 6.2 | 3.6 | 2.2 | 43.7 |
| | 1289, | | | | | |
| | hu3B9_A08- | | | | | |
| | CDRH1 | | | | | |
| IL4-1290 | IL13433- | 110.0 | 1.1 | 0.6 | 37.3 | 2.6 |
| | 1290, | | | | | |
| | hu3B9_B02- | | | | | |
| | CDRH1 | | | | | |
| IL4-1291 † | IL13433- | 26.0 | 4.8 | 2.8 | 7.5 | 12.8 |
| | 1291, | | | | | |
| | hu3B9_B03- | | | | | |
| | CDRH1 | | | | | |
| IL4-1292 † | IL13433- | 27.0 | 4.6 | 2.6 | 2.0 | 48.1 |
| | 1292, | | | | | |
| | hu3B9_B08- | | | | | |
| | CDRH1 | | | | | |
| IL4-1293 † | IL13433- | 36.0 | 3.5 | 2.0 | 2.8 | 34.4 |
| | 1293, | | | | | |
| | hu3B9_B11- | | | | | |
| | CDRH1 | | | | | |
| IL4-1294 † | IL13433- | 40.0 | 3.1 | 1.8 | 3.0 | 32.1 |
| | 1294, | | | | | |
| | hu3B9_C08- | | | | | |
| | CDRH1 | | | | | |
| IL4-1295 ⋄ | IL13433- | 51.0 | 2.4 | 1.4 | 13.0 | 7.4 |
| | 1295, | | | | | |
| | hu3B9_C09- | | | | | |
| | CDRH1 | | | | | |
| IL4-1296 | IL13433- | 150.0 | 0.8 | 0.5 | 106.0 | 0.9 |
| | 1296, | | | | | |
| | hu3B9_D04- | | | | | |
| | CDRH1 | | | | | |
| IL4-1297 † | IL13433- | 64.0 | 2.0 | 1.1 | 6.4 | 15.0 |
| | 1297, | | | | | |
| | hu3B9_F02- | | | | | |
| | CDRH1 | | | | | |
| IL4-1298 † | IL13433- | 120.0 | 1.0 | 0.6 | 4.2 | 22.9 |
| | 1298, | | | | | |
| | hu3B9_G07- | | | | | |
| | CDRH1 | | | | | |
| IL4-1299 § | IL13433- | 280.0 | 0.4 | 0.25 | 269.0 | 0.4 |
| | 1299, | | | | | |
| | hu3B9_G10- | | | | | |
| | CDRH1 | | | | | |
| IL4-1300 | IL13433- | ND | ND | ND | 23.8 | 4.0 |
| | 1300, | | | | | |
| | hu3B9_H06- | | | | | |
| | CDRH1 | | | | | |
| IL4-1301 | IL13433- | 130.0 | 1.0 | 0.55 | 146.0 | 0.7 |
| | 1301, | | | | | |
| | hu3B9_H07- | | | | | |
| | CDRH1 | | | | | |
| IL4-1302 | IL13433- | 47.0 | 2.7 | 1.5 | 53.5 | 1.8 |
| | 1302, | | | | | |
| | hu3B9_A03- | | | | | |
| | CDRH2 | | | | | |
| IL4-1303 | IL13433- | 42.0 | 3.0 | 1.7 | 32.8 | 2.9 |
| | 1303, | | | | | |
| | hu3B9_A09- | | | | | |
| | CDRH2 | | | | | |
| IL4-1304 | IL13433- | 99.0 | 1.3 | 0.7 | 70.7 | 1.4 |
| | 1304, | | | | | |
| | hu3B9_D01- | | | | | |
| | CDRH2 | | | | | |

ND = not determined;
§ = variants with substantial loss of bioactivity;
† = substantial (≥10-fold) increase in potency to neutralize CD23 expression;
⋄ = modest (5-10-fold) increase in potency to neutralize CD23 expression.

### Example 5

### Identification and Removal of Tyrosine Sulfation Post-Translational Modification of IL4-1285 Derived Variants and Generation of Anti-IL-4 Antibody IL4-0002

Purification of the humanized IL4-1285 antibody using preparative anion exchange chromatography (MonoQ, GE Healthcare) unexpectedly resulted in three distinct peaks (P1, P2 and P3) with P1, P2 and P3 representing 63%, 23% and 3% of the total eluted pool, respectively. Analytical anion exchange showed that the IL4-1285 antibody contained an acidic charged species. To further evaluate this acidic charged species, peaks P1 and P2 were successfully separated by preparative strong anion exchange (SAX) for the analysis. Results indicated that peak 2 (P2) had increased potency in bioassays and higher affinity to IL-4 relative to the main SAX peak (P1). Furthermore, P2 was shown to be partially sulfated, while P1 was not sulfated and the third peak (P3) was fully sulfated.

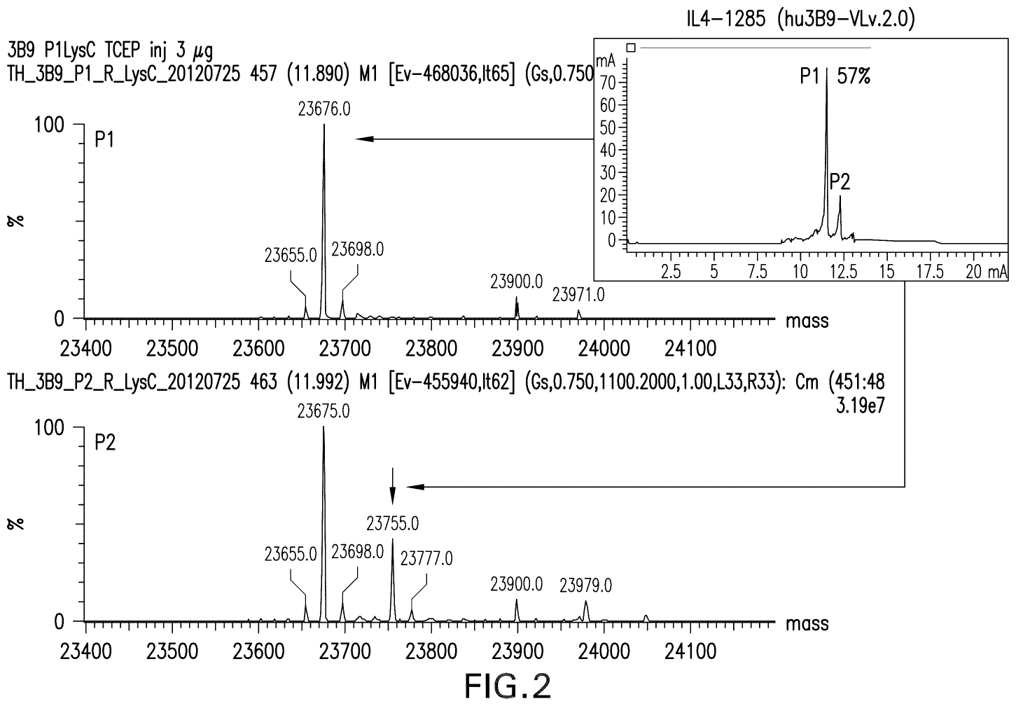

Liquid Chromatography Mass spectrometry (LC/MS) analysis was used to identify the post-translational modification position(s) responsible for the loss in bioactivity and binding affinity for IL-4. LysC digest and TCEP (tris(2-carboxyethyl) phosphine) reduction was performed on IL4-1285 antibody to generate three species: light chain antigen binding fragment (Fab), heavy chain Fab and fragment crystallizable (Fc) region. MS analysis using the Waters Xevo Q-TOF instrument showed that the modification was 80 daltons (Da) in size and located on the light chain (FIG. 2). Further evaluation using a Thermo Orbitrap XL mass spectrometer indicated that it was a Tyrosine (Tyr) at position L27d (Pfabat numbering) in the V_{L} CDR1 that was partially sulfated.

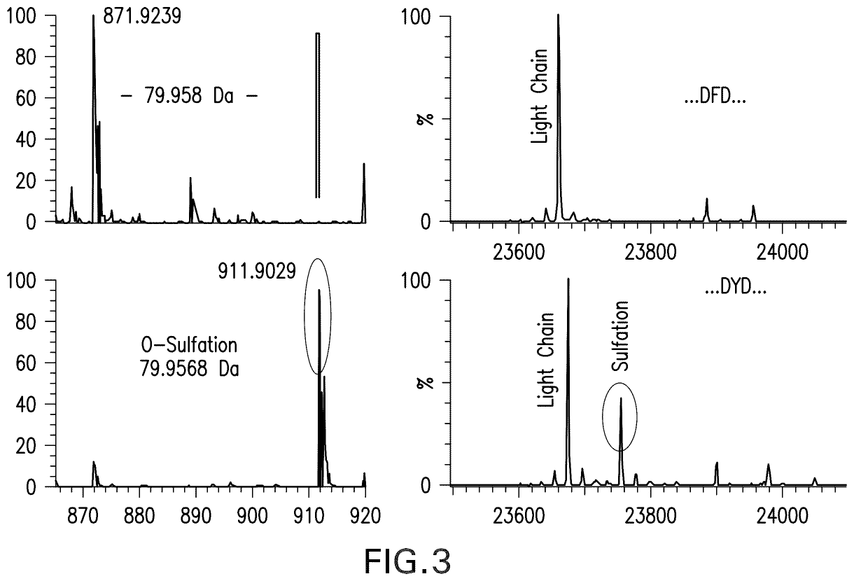

Efforts were undertaken to engineer the IL4-1285 CDRL1 (KASQSVDYDGDSYMN, SEQ ID NO: 11) to remove the O-sulfation post-translational modification occurring at tyrosine residue L27d (Pfabat numbering) in the anti-IL-4 antibody. Protein structure modeling of the IL4-1285 scFv with IL-4 co-crystal complex suggested that this tyrosine residue could contribute to cytokine binding and led to the recommendation of using either asparagine (IL4-1345 V_{L} or hu3B9-VL v2.7, V_{L}), phenylalanine (IL4-1346 V_{L} or hu3B9_VL v2.8 V_{L}) or glutamic acid (IL4-1347 V_{L} or hu3B9_VL v2.9 V_{L}, SEQ ID NO: 20) as a replacement for the tyrosine residue at L27d in the IL4-1285 VL v2.0. Additionally, the aspartic acid residue at L28 (Kabat numbering) was changed to glutamic acid (IL4-1348 V_{L}, hu3B9_VL v2.10 V_{L}) to evaluate degree of sulfation on Tyr (L27d). These modified anti-IL-4 IL4-1285 V_{L} cDNA were joined with the human Kappa constant region within an expression vector to generate the light chains (LC) for evaluation of post-translational modification and are designated IL4-1345 LC, IL4-1346 LC, IL4-1347 LC (SEQ ID NO: 197) and IL4-1348 LC. DNA encoding these modified lights chains (IL4-1345 LC, IL4-1346, r IL4-1347 LC, or IL4-1348 LC) containing amino acid substitutions to putatively prevent the partial sulfation of Tyr (L27d) were co-transfected with the IL4-1285 HC (SEQ ID NO: 10) into HEK-293 cells and protein was purified for further characterization. LC/MS analysis showed that the Tyr(27d)Phe mutation successfully eliminated the sulfated Tyrosine post-translational modification (FIG. 3).

Bioactivity was assessed for the IL4-1285 derived antibodies harboring the light chain variants engineered to remove the post-translational modification that results in partial sulfation of Y(L27d) using the CD23 expression assay with primary human monocytes previously described herein. DNA encoding the IL4-1285 modified lights chains were transiently co-transfected in HEK-293 cells with either the IL4-1285 heavy chain or the anti-IL-4 affinity optimized heavy chain variants and resultant protein was evaluated for inhibitory activity of CD23 expression relative to the IL4-1285 antibody. Substitution of either phenylalanine (IL4-1346 or hu3B9-VLv2.8) or glutamic acid (IL4-1347 or hu3B9-VLv2.9) for this tyrosine residue when combined with the parental L4-1285 heavy chain both retained activity in the monocyte 0023 bioassay as compared to parental IL4-1285 antibody (Table 4). Anti-IL-4 light chains with substitution of Y(L27d) for an asparagine residue (IL4-1345 or hu3B9-VLv2.7) or mutation of aspartic acid (L28) to glutamic acid (VL4-1348 or hu3B9-VLv2.10) when combined with L4-1285 heavy chain also retained activity, but were not as potent as L4-1346 and IL4-1347 variants (Table 4). Combining top-ranked anti-IL-4 antibody heavy chains derived from the affinity maturation process with either L4-1346 or IL4-1347 resulted in 2-24-fold enhanced potency relative to the parental anti-IL-4 antibody IL4-1285 (Table 4).

TABLE 4
Anti-IL-4 Antibody Variants Harboring Engineered Light Chains for Removal
of Partial Tyrosine Sulfation Post-Modification Retain Potency

| Anti-IL-4 | | Neutralization of IL-4 | Fold bioactivity |
|---|---|---|---|
| Antibody | | Induced CD23 | relative to |
| Variant | Variant Aliases | Expression [pM] | IL4-1285 |
| IL4-1285 | IL13433-1285, hu3B9 VL | 96.2 | 1 |
| | v2.0 | | |
| IL4-1345 | IL13433-1345, hu3B9- | 559.0 | 0.2 |
| | VLv2.7 | | |
| IL4-1346 | IL13433-1346, hu3B9- | 73.4 | 1.3 |
| | VLv2.8 | | |
| IL4-1347 | IL13433-1347, hu3B9- | 65.2 | 1.5 |
| | VLv2.9 | | |
| IL4-1348 | IL13433-1348, hu3B9- | 229.0 | 0.4 |
| | VLv2.10 | | |
| IL4-1349 ⋄ | IL13433-1349, | 14.8 | 6.5 |
| | hu3B9-A02_VLv2.8 | | |
| IL4-1350 ⋄ | IL13433-1350, | 17.9 | 5.4 |
| | hu3B9-A02_VL v2.9 | | |
| IL4-1351 | IL13433-1351, | 26.4 | 3.6 |
| | hu3B9-A08_VLv2.8 | | |
| IL4-1352 ⋄ | IL13433-1352, | 19.6 | 4.9 |
| | hu3B9-A08_VLv2.9 | | |
| IL4-1353 ⋄ | IL13433-1353, | 10.5 | 9.2 |
| | hu3B9-B08_VLv2.8 | | |
| IL4-1354 † | IL13433-1354, | 6.3 | 15.3 |
| | hu3B9-B08_VLv2.9 | | |
| IL4-1355 | IL13433-1355, | 23.4 | 4.1 |
| | hu3B9-B11_VLv2.8 | | |
| IL4-1356 | IL13433-1356, | 44.9 | 2.1 |
| | hu3B9-B11_VLv2.9 | | |
| IL4-1357 ⋄ | IL13433-1357, | 14.5 | 6.6 |
| | hu3B9-C08_VLv2.8 | | |
| IL4-1358 ⋄ | IL13433-1358, | 16.8 | 5.7 |
| | hu3B9-C08_VLv2.9 | | |
| IL4-1322 † | IL13433-1322, | 7.2 | 13.4 |
| | hu3B9-G07_VLv2.8 | | |
| IL4-1359 † | IL13433-1359, | 4.0 | 24.0 |
| | hu3B9-G07_VLv2.9 | | |

† = substantial (≥10-fold) increase in potency to neutralize CD23 expression;
⋄ = modest (5-10-fold) increase in potency to neutralize CD23 expression.

IL4-1359 anti-IL-4 antibody variant was modified to reduce spontaneous cyclization of the heavy chain N-terminal glutamine (Q) residue to pyroglutamate by substituting Q(H1) with glutamic acid (E) yielding IL4-1305 (hu3B9_G07-VLv2.9). Anti-IL-4 antibody IL4-1305 was further engineered to include a germline JH6 region (SEQ ID NO: 21) by substituting the arginine (R) at H105 for Q and the phenylalinine (F) at H108 for threonine (T) to generate IL4-0002 antibody.

### Example 6

### Engineering Anti-IL-4 IL4-0002 Antibody to Remove Post-Translational Isomerization Modification and Reduce Viscosity

The antigen-binding fragment (Fab) derived from the IL4-0002 antibody variant was incorporated into the tri-Fab-Fc, IL13433-0006, to neutralize IL-4 activity. The CDRL1 of anti-IL-4 antibody IL4-0002 contains a “DG” motif at D28/G29 that is frequently a hot-spot for post-translation isomerization modification. Forced degradation analysis was performed on the IL13433-0006 tri-Fab-Fc to interrogate presence of physical or chemical liabilities. Specifically, the IL13433-0006 tri-Fab-Fc was formulated at 5 mg/mL in three different buffers, Tris pH 7.5, Histidine pH 5.8 and Glutamic acid pH 4.5. Next, the formulated IL13433-0006 tri-Fab-Fc samples were incubated at 40° C. and aliquots were removed at 2 and 4 weeks. LC/MS peptide mapping analysis was used to determine the level of post-translation modification for the Glutamic acid pH 4.5 and Tris pH 7.5 IL13433-0006 tri-Fab-Fc forced degradation samples. For this method, the forced degradation samples were subjected to double digestion with LysC and Trypsin and MS analysis was done using a high-fidelity method with a Lumos C18 Column. A post-translational isomerization modification was detected in the anti-IL-4 antibody IL4-0002 light chain CDR1 peptide (spanning A25-K42) at D(L28). The 4-week (T4) Glutamic acid pH 4.5 sample has highest level of isomerization at 78.3%, however the 4-week (T4) Tris pH 7.5 sample also showed significant isomerization at 55.1% relative to the 5.9% isomerization detected for the time zero (TO) sample formulated in PBS-CMF (Table 5).

TABLE 5
Percent Post-Translational Isomerization Modification
Detected by LC/MS Peptide Mapping

| | | % | % Total | |
|---|---|---|---|---|
| | % IsoD | Succinimide | Isomeriza- | % Native |
| Sample | Peptide | Peptide | tion | Peptide |
| IL13433-0006 | 3.8 | 1.9 | 5.9 | 94.3 |
| PBS-CMF (T0) | | | | |
| IL13433-0006 | 54.3 | 2.0 | 55.1 | 45.1 |
| Tris pH 7.5 (T4) | | | | |
| IL13433-0006 | 61.8 | 18.2 | 78.3 | 23.0 |
| Glutamic acid | | | | |
| pH 4.5 (T4) | | | | |

The IL4-1285 Fab/IL-4 complex structure was used to propose amino acid modifications that would remove the post-translational isomerization liability at D(L28) in CDRL1 of IL4-1285 derived antibody variants. Using the IL4-1285 Fab/IL-4 complex structure results, protein structure modeling suggested that this Asp residue could contribute to cytokine binding and led to the recommendation of substituting Glutamic acid (Glu, E) at this position. D(L28) in CDRL1 was mutated to E (D28E) and incorporated into IL4-0002 as well as the K(L24)R mutation for removing a in silico predicted T-cell epitope, assimilation of these changes results in IL4-0749 antibody. IL4-0749 antibody variant retained ability to neutralize IL-4 induced CD23 expression on primary human monocytes thus demonstrating that the D(L28)E substitution was tolerated (Table 6). IL4-0749 also harbors the K(L24)R mutation intended to remove a predicted T-cell epitope within CDRL1 and presence of the K(L24)R change was investigated by introducing only this substitution into IL4-0002 thus generating IL4-0754 antibody variant. IL4-0754 antibody completely retains ability to neutralize IL-4 relative to IL4-0002 in the CD23 bioassay demonstrating that this CDRL1 amino acid substitution does not alter IL-4 binding properties (Table 6) and supports the observation that this residue does not contact the cytokine as indicated by the IL4-1285 Fab/IL-4 complex structure results. Incorporation of D(L28)E into the IL4-0002 in addition to the K(L24)R, N(H60)S, P(H61)T, S(H65)T mutations for reducing in silico predicted T-cell epitopes plus N(L92)H and E(L93)K to lower viscosity resulted in the IL4-0157 antibody variant (SEQ ID NO: 28 and SEQ ID NO: 29, VH and VL, respectively). The combined mutations within IL4-0157 antibody contributed to an ˜5-fold lower ability to neutralize IL-4 in the CD23 bioassay relative to IL4-0002 (Table 6). Examination of individual mutations combined into IL4-0157 antibody were investigated to determine which mutation or combinations of mutations were contributing to the reduced ability of this variant to neutralize IL-4. IL4-0037 antibody was generated by incorporating the N(H60)S, P(H61)T, S(H65)T amino acid substitutions within CDR2 of the IL4-0002 heavy chain variable region and this variant was able to effectively neutralize IL-4 bioactivity relative to IL4-0002 (Table 6). However, combination of the heavy chain harboring N(H60)S, P(H61)T, S(H65)T mutations with any light chain containing N(L92)H and E(L93)K substitutions to lower viscosity results in a reduced ability to neutralize IL-4 in the CD23 bioassay (Table 6). Examination of IL4-0002 antibody engineered lights chains containing either N(L92)H, E(L93)K or N(L92)H plus E(L93)K show that the E(L93)K mutation only does not alter ability of this antibody variant to neutralize IL-4 induced CD23 expression on primary monocytes, but substitution of N(L92)H alone or in combination with E(L93)K reduces IL-4 binding properties (Table 6).

TABLE 6
Evaluation of IL-4 Neutralization in the CD23 Expression
Bioassay for Variants Engineered to Remove Isomerization
Liability and to Reduce Viscosity

| | | | Neutralization of | Fold |
|---|---|---|---|---|
| | | | IL-4 Induced | bioactivity |
| Antibody | Mutations versus IL4-0002 | | CD23 Expression | relative to |
| Variant | V_{H} | V_{L} | [pM] | IL4-0002 |
| IL4-0002 | — | — | 2.1 | 1 |
| IL4-0037 | N60S, P61T, | — | 1.6 | 1.3 |
| | S65T | | | |
| IL4-0092 | — | K24R, N92H, | 3.3 | 0.6 |
| | | E93K | | |
| IL4-0093 | N60S, P61T, | K24R, N92H, | 4.1 | 0.5 |
| | S65T | E93K | | |
| IL4-0157 | N60S, P61T, | K24R, D28E, | 9.3 | 0.2 |
| | S65T | N92H, E93K | | |
| IL4-0749 | — | K24R, D28E | 2.4 | 0.9 |
| IL4-0751 | — | N92H, E93K | 6.6 | 0.3 |
| IL4-0752 | — | N92H | 5.1 | 0.4 |
| IL4-0753 | — | E93K | 2.5 | 0.8 |
| IL4-0754 | — | K24R | 1.9 | 1.1 |
| IL4-1040 | — | K24R, D28E, | 3.2 | 0.7 |
| | | E93K | | |

No mutations (—)

### Example 7

### E(L93)K Light Chain CDR3 Substitution in IL4-0002 Derived Antibody Variants Reduces Viscosity

Viscosity of formulated IL4-0002 derived antibody variants was evaluated. The Anton Paar method was employed to assess viscosity. Specifically, protein samples were concentrated to a target of 170 mg/mL using 50 kDa molecular weight cut-off Amicon centrifugal filter units (EMD Millipore, Billerica, MA). For each protein, a set of samples ranging from 25-160 mg/mL were serially diluted using Histidine-sucrose pH 5.8 buffer as diluent. Protein concentrations were determined by 280 nm analysis on the SoloVPE Variable Pathlength System (C Technologies, Inc, Bridgewater, NJ). Viscosity measurements were performed using the CP25-1 cone and plate on the MCR-302 rheometer (Anton Paar USA Inc., Ashland, VA) at a constant rotational speed of 150 rpm at 25° C. A total of 10 measurements of 10 seconds each were collected per sample and the data was analyzed using the Rheoplus (Anton Paar USA Inc.) V 3.62 software. The Anton Paar viscosity analysis shows that the IL4-0751 and IL4-0753 variants which include the E(L93)K substitution have reduced viscosity relative to IL4-0002. Further, the E(L93)K mutation is responsible for improving the viscosity profile since the N(L92)H mutation only in IL4-0002 does not contribute to viscosity reduction (Table 7). Viscosities of 20 cP and lower are typically used in SC injections. In some situations, viscosities of between 15 cP and 20 cP are most advantageous, as extremely low viscosities can be painful at injection site.

TABLE 7
Anton Paar Viscosity Analysis of IL4-
0002 Derived Antibody Variants.

| | | Concentration @ 20 cP |
|---|---|---|
| Antibody Variant | Mutation(s) | (mg/mL) |
| IL4-0002 | — | ~72 |
| IL4-1305 | Q(H105)R, T(H108)P | ~95 |
| IL4-0749 | K(L24)R, D(L28)E | ~60 |
| IL4-0751 | N(L92)H, E(L93)K | ~122 |
| IL4-0752 | N(L92)H | ~82 |
| IL4-0753 | E(L93)K | ~126 |

### Derivation of Anti-IL-13 Fab Binding Domain

### Example 8

### Isolation of a Mouse Monoclonal Antibody IL13-1306 that Binds and Neutralizes Human IL-13 Activity

Polyclonal antisera were prepared by immunization of female BALB/c mice with recombinant human IL-13 (R&D Systems, Minneapolis, Minn.). Sera were screened for binding to human IL-13 by ELISA. Splenocytes from a mouse demonstrating high serum antibody titers were fused with the P3X63_AG8.653 myeloma (ATCC) and plated in selective media. Fusions were isolated with three rounds of sub-cloning by limiting dilution and screened for the production of antibodies that had a binding affinity to human IL-13. Three monoclonal antibodies were capable of binding IL-13, interfering with the formation of a functional IL-13 signaling complex and neutralizing one or more IL-13-associated activities. Monoclonal antibody IL13-1306 (mu13.4) was chosen for humanization based on its potent cytokine neutralization activity and favorable epitope.

### Example 9

### Cloning Mouse Monoclonal Antibody IL13-1306 Heavy and Light Chain Variable Regions

The IL13-1306 (mu13.4) anti-IL-13 antibody heavy chain and light chain variable regions were cloned using the SMART® cDNA synthesis system (Clontech Laboratories Inc. of Mountain View, CA) followed by PCR amplification. The cDNA was synthesized from 1 μg total isolated from IL13-1306 hybridoma cells, using oligo (dT) and the SMART® IIA oligo (Clontech Laboratories Inc.) with POWERSCRIPT™ reverse transcriptase (Clontech Laboratories Inc.). The cDNA was then amplified by PCR using a primer which anneals to the SMART® IA oligo sequence and mouse constant region-specific primer (mouse Kappa for the light chain and mouse IgG1 for the heavy chain) with VENT® polymerase (New England Biolabs Inc. of Ipswich, MA). Heavy and light chain PCR products were subcloned into the pED6 expression vector and the nucleic acid sequence was determined. This method is advantageous in that no prior knowledge of the DNA sequence is required. In addition, the resultant DNA sequence is not altered by use of degenerate PCR primers. The amino acid sequence of the IL13-1306 (mu13.4) heavy chain variable region is set forth as SEQ ID NO: 44. The amino acid sequence of the IL13-1306 (mu13.4) light chain variable region is set forth as SEQ ID NO: 46.

### Example 10

### Humanization of Mouse Anti-IL-13 Antibody IL13-1306

Mouse IL13-1306 (mu13.4) anti-IL-13 antibody was humanized by Complementarity Determining Region (CDR) grafting as described further herein below. The CDRs of mouse IL13-1306 antibody were identified using the Pfabat numbering definition (EXAMPLE 1). A humanized heavy chain variable region (V_{H}) was constructed to include the CDRs of mouse IL13-1306 V_{H} (SEQ ID NO: 41, SEQ ID NO: 42 and SEQ ID NO: 43) grafted onto a human IGHV3-7*01 (DP-54) framework region from the VH3 subgroup with a JH4 segment (SEQ ID NO: 47) and this amino acid sequence is set forth as SEQ ID NO: 48, IL13-1307 (hu13.4) V_{H}. Similarly, the IGKV1D-39*01 (DPK9) human germline acceptor framework from the VKI sub-group was used to engineer a CDR grafted version of humanized light chain variable region with a JK4 segment (SEQ ID NO: 14) and this amino acid sequence is set forth in SEQ ID NO: 49 IL13-1307 (hu13.4) V_{L}. The humanized IL13-1307 (hu13.4) V_{H} (SEQ ID NO: 48) was joined to the human IgG1 constant region that was modified to diminish effector function (L247A, L248A and G250A, Pfabat numbering; SEQ ID NO: 6, SEQ ID NO: 7, SEQ ID NO: 8 and SEQ ID NO: 9) thus generating IL13-1307 (hu13.4) HC and sub-cloned into an expression vector. The humanized IL13-1307 V_{L} (hu13.4 VL, SEQ ID NO: 49) was fused to the human Kappa constant region (SEQ ID NO: 16) to create hu13.4 LC and sub-cloned into an expression vector. IL-13 bioactivity was determined using the pSTAT6 phosphorylation assay (see method in EXAMPLE 3). For assays testing antibody inhibition of IL-13 cytokine responses, recombinant human IL-13 (1 ng/ml; R&D Systems) was added along with dilutions of antibodies ranging from 500 to 0.4 ng/mL.

Humanized IL13-1307 antibody retains ability to neutralize IL-13 induced CD23 expression on primary monocytes and pSTAT6 phosphorylation relative to the parental mouse monoclonal antibody IL13-1306 and the benchmark antibody IL13-1283 (Table 8).

TABLE 8
Humanized IL13-1307 antibody retains ability
to neutralize IL-13 induced CD23 expression
on primary monocytes and pSTAT6 phosphorylation

| | Neutralization of IL- | |
|---|---|---|
| | 13-induced pSTAT6 | Neutralization of IL- |
| | Phosphorylation | 13 Induced CD23 |
| Antibody | [pM] | Expression [pM] |
| IL13-1307 (hu13.4) | 224.6 | 213.9 |
| IL13-1283 (IMA-638) | 427.1 | 260.8 |
| IL13-1306 (mu13.4) | 290.5 | ND |

ND = Not determined.

### Example 11

### Engineering Humanized IL13-1307 Antibody to Increase Binding Affinity and Potency of Neutralization for the IL-13 Cytokine

Humanized IL13-1307 (hu13.4) antibody underwent engineering efforts to increase IL-13 binding affinity in order to improve potency of IL-13 cytokine neutralization. To facilitate phage display, the variable domain regions of IL13-1307 antibody (SEQ ID NO: 48 and SEQ ID NO: 49) were cloned in a single-chain variable fragment (scFv) format using a V_{L}-V_{H} orientation with a (G4S)3 flexible linker within the phage-display vector pWRIL-1. For IL13-1307, a three-tiered approach was designed to encompass different library designs and maximize the number of hits obtained. In the first approach, random ‘soft’-style mutagenesis was performed, which leaves ˜50% parental wild-type residue in the library at each targeted position, plus random amino acid content in the other 50%. The second approach that was taken involved the use of ‘homolog scanning’ degenerate oligonucleotides, which swap each wild-type residue for structurally homologous amino acids, with two possibilities per position. The third mutagenesis approach used a library built with amino acids chosen at each CDR position based on the statistical analysis of antibody sequences as well as computational and manual inspection of the binding interface in the co-crystal structure of the IL13-1307 (hu13.4) antibody with IL-13 (EXAMPLE 14). A two round selection campaign was employed for each of the sub libraries, involving an extremely stringent initial first round of selection against 100 pM biotinylated human IL-13 with an incorporated overnight ‘off-rate’ challenge with a 1000-fold excess (100 nM) of human IL-13. In the second round, 50 pM biotinylated human IL-13 was used without any ‘off-rate’ challenge.

A panel of clones was randomly picked from each round of phage selection outputs for the anti-IL-13 selections. Analysis of these selected clones was performed via induction of scFv expression and the use of E. coli peripreps in a homogeneous time resolved fluorescence (HTRF) competition assay. Unique clones which showed increased competition in comparison to the parental IL13-1307 scFv were identified and taken forward for further analysis. These clones were reformatted to full-length IgG1-effector function-reduced/human Kappa antibodies and proteins were produced transiently in HEK-293 cells for further evaluation.

Variants selected based on improved IL-13 binding activities in the HTRF competition assay were monitored by bioassay for increased bioactivity. Because it had the greatest sensitivity range, the CD23 expression assay with primary human monocytes was primarily used for this analysis. Specifically, mononuclear cells were isolated from human peripheral blood by layering over Histopaque (Sigma Aldrich). Cells were washed into RPMI medium containing 10% heat-inactivated Fetal Calf Serum, 50 U/mL penicillin, 50 μg/mL streptomycin, 2 mM L-glutamine, and plated in a 48-well tissue culture plate (Costar/Corning). Recombinant human IL-13 was added at dilutions ranging from 100 to 0.01 ng/mL. For assays testing antibody inhibition of cytokine responses, 1 ng/mL IL-13 was added along with dilutions of the antibodies ranging from 500 to 0.4 ng/mL. Cells were incubated overnight at 37° C. in a 5% CO₂ incubator. The next day, cells were harvested from wells using non-enzymatic Cell Dissociation Solution (Sigma Aldrich), and then washed into ice-cold PBS containing 1% BSA. Cells were incubated with phycoerythrin (PE)-labeled antibody to human CD23 (BD Biosciences), and Cy-Chrome-labeled antibody to human CD11b (BD Biosciences). Monocytes were gated based on high forward and side light scatter, and expression of CD11b. CD23 expression on monocytes was determined by flow cytometry using a flow cytometer (BD Biosciences), and the percentage of CD23-positive cells was analyzed with CellQuest software (BD Biosciences). Because the CD23 expression assay is run with human peripheral blood, the monocyte CD23 expression assay shows subtle variations in response based on donor. Therefore, the responses of the affinity optimized variants were compared to IL13-1307 (hu13.4) parental antibody controls run in each individual assay. Data are expressed as % maximal response, which typically ranged from 65-85% C023+ monocytes. Neutralization of IL-13-induced STAT6 phosphorylation bioactivity in HT-29 cells was also measured for select variants as a comparator to parental IL13-1307 (hu13.4) antibody. Several anti-IL-13 affinity optimized variants were identified that showed a significant 10-46-fold increase in potency of IL-13 neutralization in the 0023 expression assay and modest improvement in neutralizing IL-13 induced pSTAT6 phosphorylation bioactivity in HT-29 cells since this assay window is narrow (Table 9).

TABLE 9
Select Affinity Matured Anti-IL-13 Antibody Variants Exhibit a Significant
Increase in Potency of IL-13 Neutralization in the CD23 Expression Bioassay and Modest Improvement
in pSTAT6 Bioactivity

| | | Neutralization of IL-13-induced | | | Neutralization of IL-13 Induced | | |
|---|---|---|---|---|---|---|---|
| | | pSTAT6 Phosphorylation | | | CD23 Expression | | |
| Anti-IL- | | | Fold | Fold | | Fold | Fold |
| 13 | | | bioactivity | bioactivity | | bioactivity | bioactivity |
| Antibody | Variant | IC₅₀ | relative to | relative to | IC₅₀ | relative to | relative to |
| Variant | Aliases | [pM] | IL13-1307 | IL13-0001 | [pM] | IL13-1307 | IL13-0001 |
| IL13- | IL13433- | 140.0 | 1 | 0.7 | 305.9 | 1 | 0.03 |
| 1307 | 1307, | | | | | | |
| | hu13.4 | | | | | | |
| IL13- | IL13433- | 92.0 | 1.5 | 1 | 10.5 | 29.1 | 1 |
| 0001 † | 0001 | | | | | | |
| IL13- | IL13433- | 42.7 | 3.3 | 2.2 | 14.4 | 21.3 | 0.7 |
| 1308 † | 1308, | | | | | | |
| | VLA4 | | | | | | |
| IL13- | IL13433- | 50.00 | 2.8 | 1.8 | 16.31 | 18.8 | 0.6 |
| 1309 † | 1309, | | | | | | |
| | VLA6 | | | | | | |
| IL13- | IL13433- | 41.33 | 3.4 | 2.2 | 9.35 | 32.7 | 1.1 |
| 1310 † | 1310, | | | | | | |
| | VLA7 | | | | | | |
| IL13- | IL13433- | 54.50 | 2.6 | 1.7 | 16.7 | 18.3 | 0.6 |
| 1311 † | 1311, | | | | | | |
| | VLA 10 | | | | | | |
| IL13- | IL13433- | 34.3 | 4.1 | 2.7 | 9.8 | 31.2 | 1.1 |
| 1312 † | 1312, | | | | | | |
| | VLA 12 | | | | | | |
| IL13- | IL13433- | 60.0 | 2.3 | 1.5 | 14.8 | 20.6 | 0.7 |
| 1313 † | 1313, | | | | | | |
| | VLB6 | | | | | | |
| IL13- | IL13433- | 52.3 | 2.7 | 1.8 | 8.8 | 35.0 | 1.2 |
| 1314 † | 1314, | | | | | | |
| | VLB11 | | | | | | |
| IL13- | IL13433- | 77.0 | 1.8 | 1.2 | 26.65 | 11.5 | 0.4 |
| 1315 † | 1315, | | | | | | |
| | VLB12 | | | | | | |
| IL13- | IL13433- | 36.5 | 3.8 | 2.5 | 8.8 | 34.8 | 1.2 |
| 1316 † | 1316, | | | | | | |
| | VLC7 | | | | | | |
| IL13- | IL 13433- | 71.0 | 2.0 | 1.3 | 28.4 | 10.8 | 0.4 |
| 1317 † | 1317, | | | | | | |
| | VLC12 | | | | | | |
| IL13- | IL13433- | 310.0 | 0.5 | 0.3 | 101 | 3.0 | 0.1 |
| 1318 | 1318, | | | | | | |
| | 1RVLC1 | | | | | | |
| IL13- | IL13433- | 110.0 | 1.3 | 0.8 | 19.8 | 15.4 | 0.5 |
| 1319 † | 1319, | | | | | | |
| | 1RVLE3 | | | | | | |
| IL13- | IL13433- | 190.0 | 0.7 | 0.5 | 25.9 | 11.8 | 0.4 |
| 1320 † | 1320, | | | | | | |
| | 3RVLA11 | | | | | | |
| IL13- | IL13433- | 120.0 | 1.2 | 0.8 | 22.4 | 13.7 | 0.5 |
| 1321 † | 1321, | | | | | | |
| | 1RVLG2 | | | | | | |
| IL13- | IL13433- | 170.0 | 0.8 | 0.5 | 29.7 | 10.3 | 0.4 |
| 1323 † | 1323, | | | | | | |
| | 1RVLA3 | | | | | | |
| IL13- | IL13433- | 180.0 | 0.8 | 0.5 | 36.8 | 8.3 | 0.3 |
| 1324 ⋄ | 1324, | | | | | | |
| | 1RVLB11 | | | | | | |
| IL13- | IL13433- | 150.0 | 0.9 | 0.6 | 33.2 | 9.2 | 0.3 |
| 1325 ⋄ | 1325, | | | | | | |
| | 1RVLG3 | | | | | | |
| IL13- | IL13433- | 130.0 | 1.1 | 0.7 | 25.4 | 12.0 | 0.4 |
| 1326 † | 1326, | | | | | | |
| | 1RVLC7 | | | | | | |
| IL13- | IL13433- | 160.0 | 0.9 | 0.6 | 15.8 | 19.4 | 0.7 |
| 1327 † | 1327, | | | | | | |
| | 1RVLC10 | | | | | | |
| IL13- | IL13433- | 190.0 | 0.7 | 0.5 | 26.3 | 11.6 | 0.4 |
| 1328 † | 1328, | | | | | | |
| | 2RVLF12 | | | | | | |
| IL13- | IL13433- | 67.0 | 2.1 | 1.4 | 11.6 | 26.3 | 0.9 |
| 1329 † | 1329, | | | | | | |
| | 1VHC4 | | | | | | |
| IL13- | IL13433- | 120.0 | 1.2 | 0.8 | 80.3 | 3.8 | 0.1 |
| 1330 | 1330, | | | | | | |
| | 2VHD4 | | | | | | |
| IL13- | IL13433- | 150.0 | 0.9 | 0.6 | 57.5 | 5.3 | 0.2 |
| 1331 ⋄ | 1331, | | | | | | |
| | 1VHD12 | | | | | | |
| IL13- | IL13433- | 53.5 | 2.6 | 1.7 | 20.9 | 14.6 | 0.5 |
| 1332 † | 1332, | | | | | | |
| | 1VHC6 | | | | | | |
| IL13- | IL13433- | 89.0 | 1.6 | 1.0 | 45 | 6.8 | 0.23 |
| 1333 ⋄ | 1333, | | | | | | |
| | 1VHC9 | | | | | | |
| IL13- | IL13433- | 71.0 | 2.0 | 1.3 | 12.3 | 24.8 | 0.8 |
| 1334 † | 1334, | | | | | | |
| | 2VHE12 | | | | | | |
| IL13- | IL13433- | 150.0 | 0.9 | 0.6 | 42.2 | 7.2 | 0.2 |
| 1335 ⋄ | 1335, | | | | | | |
| | 1RVHA9 | | | | | | |
| IL13- | IL13433- | 160.0 | 0.9 | 0.6 | 50.6 | 6.0 | 0.21 |
| 1336 ⋄ | 1336, | | | | | | |
| | 1RVHB12 | | | | | | |
| IL13- | IL13433- | 120.0 | 1.2 | 0.8 | 15.0 | 20.4 | 0.7 |
| 1337 † | 1337, | | | | | | |
| | 1RVHB8 | | | | | | |
| IL13- | IL13433- | 87.0 | 1.6 | 1.1 | 11.5 | 26.5 | 0.9 |
| 1338 † | 1338, | | | | | | |
| | 1RVHC9 | | | | | | |
| IL13- | IL13433- | 140.0 | 1.0 | 0.7 | 33.7 | 9.1 | 0.3 |
| 1339 ⋄ | 1339, | | | | | | |
| | 1RVHC10 | | | | | | |
| IL13- | IL13433- | 190.0 | 0.7 | 0.5 | 25.4 | 12.0 | 0.4 |
| 1340 † | 1340, | | | | | | |
| | 1RVHD7 | | | | | | |
| IL13- | IL13433- | 170.0 | 0.8 | 0.5 | 97.9 | 3.1 | 0.1 |
| 1341 | 1341, | | | | | | |
| | 1RVHD12 | | | | | | |
| IL13- | IL13433- | 150.0 | 0.9 | 0.6 | 47.9 | 6.4 | 0.2 |
| 1342 ⋄ | 1342, | | | | | | |
| | 1RVHE6 | | | | | | |
| IL13- | IL13433- | 81.0 | 1.7 | 1.1 | 46.5 | 6.6 | 0.2 |
| 1343 ⋄ | 1343, | | | | | | |
| | 1RVHE7 | | | | | | |
| IL13- | IL13433- | 170.0 | 0.8 | 0.5 | 20.4 | 15.0 | 0.5 |
| 1344 † | 1344, | | | | | | |
| | 2RVHF10 | | | | | | |

ND = not determined;
† = variants with substantial (≥10-fold) increase in potency to neutralize CD23 expression;
⋄ = modest (5-10-fold) increase in potency to neutralize CD23 expression.

### Example 12

### Removal of In Silico Predicted Epitopes from Anti-IL-13 Antibody IL13-0001

Efforts were undertaken to reduce in silico predicted non-germline T cell epitope content in IL13-0001 CDRs to potentially reduce overall immunogenicity profile of Tri-Fab-Fc molecule harboring this binding domain. The immunogenicity analysis was performed using the following two methods. IL13-0001 V_{H} and V_{L} sequences were submitted for EpiMatrix analysis in the ISPRI software package (ISPRI v 1.8.0, EpiVax Inc., Providence, RI; 26) and the raw results provide rankings of likelihood of binding of each 9-oligomer amino acid fragment against 8 different HLA types. Sequences were also submitted for analysis using the MHC-II binding Consensus method (27) in IEDB (IEDB MHC-II Binding Predictions, Vita et al., 2015) and raw results provide rankings of the probability of binding for each 9-oligomer and 15-oligomer amino acid fragments against the same eight different HLA types. Next, each epitope is classified as a germline or non-germline epitope and for antibodies, we further classify each epitope based on its location within the antibody (CDR or non-CDR). The analysis of IL13-0001 identified seven predicted non-germline T cell epitopes, four in the V_{H} and three in the V_{L}. Amino acid substitutions were selected using a structural guided approach with both computational and manual inspection of the binding interface in the co-crystal structure of the IL13-1307 (hu13.4) antibody with IL-13 (EXAMPLE 14) to identify changes that do not interfere with the potent neutralization ability of IL13-0001. Thirty-six unique variants were engineered as IgG1-effector function-reduced/human Kappa antibodies and proteins were produced transiently in HEK-293 cells for further evaluation.

The CD23 expression assay with primary human monocytes (described in EXAMPLE 11) was used to assess bioactivity of these variants relative to IL13-0001. The affinity values (K_{D}) were determined using Surface Plasmon Resonance (SPR) for 10 lead variants identified from bioassay screening. For evaluation of kinetic rate constants to determine 1:1 binding affinity, an anti-human IgG antibody (GE Healthcare, BR-1008-39) was covalently amine coupled onto all flow cells of a CM5 carboxymethylated dextran coated sensor chip to a density of about 10,000 resonance units (RU) following the manufacturer's protocol and then anti-IL-13 antibody variants were captured to a level of approximately 60-90 RUs. Next, human IL-13 protein ranging in concentration from 1.56-50 nM was injected over the surface and the surface at 37° C. was regenerated with an ionic buffer followed by equilibration with HBS-EP+. Methods used for AC-SINS, DNA and insulin non-specificity assays are described in EXAMPLE 16.

Evaluation of variants harboring amino acid substitutions introduced in IL13-0001 CDRs to reduce non-germline T cell epitope content identified substitutions that did not significantly alter IL-13 binding and neutralization properties. Specifically, variants were identified that remove the four V_{H} and two of three V_{L} predicted T cell epitopes, however minor loss in IL-13 bioactivity was noted for these top ranked clones (Table 10). Further, the affinity (K_{D}) measured for variants binding human IL-13 using SPR at 37° C. also show a minor loss of affinity for IL-13 and support bioactivity findings (Table 10). The non-specificity assessment performed using AC-SINS, DNA and insulin assays show that mutations incorporated to reduce predicted T cell epitopes do not alter estimation of the parental IL13-0001 scoring (Table 10).

TABLE 10
Summary of Bioactivity and Affinity for Lead Anti-IL-13 Variants Designed to
Reduce In Silico Predicted Immunogenicity.

| | | | | | | | Bioactivity: | | |
|---|---|---|---|---|---|---|---|---|---|
| | | | | | | | Neutralization | | |
| | | | | | | | of IL-13 | | |
| | | | | | | | Induced | | |
| | | | | | | | CD23 | | |
| | Multichain | | | | | | Expression | | |
| | Immuno- | CDR | | Non-specificity | | | IC₅₀ | Fold | |
| | genicity Score | Non-germline | | Scores | | | [pM] | versus | K_{D} [pM] |
| | (tReg- | Epitopes | | AC- | | | Ave. | IL13- | SPR |
| Antibody | Adjusted) | V_{H} | V_{L} | SINS | DNA | Insulin | n = 2 | 0001 | 37° C. |
| IL13- | −45.39 | 4 | 3 | 10.0 | 2 | 2 | 6.0 | 1 | 57.7 ± 0.2 |
| 0001 | | | | 0 | | | | | |
| IL13- | −49.33 | 0 | 2 | 11.0 | 4 | 2 | 8.2 | 0.73 | 67 ± 19.4 |
| 0258 | | | | 0 | | | | | |
| IL13- | −51.93 | 0 | 2 | 10.0 | 4 | 4 | 7.4 | 0.81 | 71.75 ± 3.45 |
| 0259 | | | | 0 | | | | | |
| IL13- | −61.30 | 0 | 1 | 6.00 | 2 | 1 | 9.2 | 0.65 | 88.8 ± 0.8 |
| 0265 | | | | | | | | | |
| IL13- | −63.90 | 0 | 1 | 8.00 | 5 | 2 | 7.9 | 0.76 | 80.3 ± 0.6 |
| 0266 | | | | | | | | | |
| IL13- | −58.80 | 0 | 1 | 5.00 | 1 | 1 | 9.0 | 0.67 | 93.25 ± 0.45 |
| 0270 | | | | | | | | | |
| IL13- | −61.40 | 0 | 1 | 8.00 | 3 | 2 | 5.9 | 1.01 | 73.55 ± 4.15 |
| 0271 | | | | | | | | | |
| IL13- | −62.60 | 0 | 1 | 7.00 | 3 | 1 | 7.4 | 0.81 | 109 ± 2 |
| 0277 | | | | | | | | | |
| IL13- | −65.20 | 0 | 1 | 8.00 | 3 | 2 | 9.6 | 0.62 | 90.9 ± 2.8 |
| 0278 | | | | | | | | | |
| IL13- | −60.10 | 0 | 1 | 8.00 | 2 | 1 | 9.6 | 0.62 | 106.5 ± 11.5 |
| 0282 | | | | | | | | | |
| IL13- | −62.70 | 0 | 1 | 8.00 | 3 | 2 | 7.0 | 0.86 | 80.45 ± 3.25 |
| 0283 | | | | | | | | | |

### Example 13

### Determination of Humanized IL4-1285 Antibody-Human IL-4 Complex Structure

To determine which residues in humanized IL4-1285 antibody (hu3B9-VLv2.0 or RA1-2) make a direct contact with human IL-4 cytokine and to enable structure-based library design, the structure was determined for the complex of IL4-1285 antibody with human IL-4. To obtain this structure, IL-4 was over-expressed in the E. coli cell line BL21(DE3). Cells were lysed through micro-fluidizer in the presence of Complete Protease Inhibitors (Merck). After centrifuging at 25,000 g the inclusion bodies were washed and re-solubilized with 6M Guanadinium-HCl (GuHCl), 20 mM DTT and 1 mM EDTA (pH 8.8). Sequential dialysis steps were performed to gradually remove GuHCl while introducing oxidized glutathione (to final concentration of 1 mM) to assist refolding. After complete removal of GuHCl, the refolded IL4 was purified through HiTrap SP Sepharose FF, HiTrap Phenyl HP, and Superdex 75 16/60 (all three columns through GE Healthcare) to obtain protein for structural studies. The anti-IL-4 IL4-1285 antibody was digested with immobilized Papain per manufacturer protocol (Thermo/Pierce). Pre-packed Protein A Sepharose FF (GE Healthcare) was used to purify Fab fragments from the digested mixture. The IL4-1285 Fab and human IL-4 cytokine (ligand) were mixed at a ratio of 1:1.2 with excess of ligand to drive complex formation. Final purification was performed using a Superdex 200 size-exclusion column (GE Healthcare). The IL4-1285 Fab/IL-4 complex was concentrated to 13.5 mg/mL for crystallization setup. Optimal crystals of the protein complex containing IL4-1285 Fab and IL-4 were obtained using the following condition: 100 mM MES pH 6.0, 150 mM Ammonium Sulfate, 14% PEG 4000. A large crystal of the IL4-1285 Fab/IL-4 complex diffracted to about 2.4 Å. Crystals were transiently cryoprotected and synchrotron data collection was performed remotely at Advanced Photon Source (APS). Image frames were processed using software AutoPROC (Global Phasing Ltd). A complete dataset was obtained at 2.40 Å resolution. The data belongs to space group P3₁21, with unit cells as follows: a=114.175 Å, b=114.175 Å, c=160.790 Å, α=ß=90°, γ=120° with 2 complexes per asymmetric unit. Molecular Replacement searches using homology model of the IL4-1285 Fab and IL-4 structures yielded convincing solutions of each component. Refinement was performed using software BUSTER, and the final R/R_{free} factors at 2.4 Å are 0.2099 and 0.2593, respectively, with RMSD of bonds 0.013 Å, RMSD of angles 1.947°.

From the analysis of these structural results, the following residues (Pfabat numbering) in the IL4-1285 heavy chain are involved in direct contact with human IL-4: S32, G33, W53, R94, E96, T97, V98, F99, Y100, Y(100B). Additionally, the following residues in the anti-IL-4 IL4-1285 Fab light chain are involved in direct contact with the IL-4 cytokine: Y(27D), D28, D30, Y32, L46, Y49, A50, E55, S56. It was also determined that the following IL-4 amino acids are involved in direct contact with the anti-IL-4 IL4-1285 Fab: E19, Q20, A68, T69, A70, Q71, F73, H74, R75, K77, Q78, R81, F82, K84, R85.

The IL4-1285 Fab/IL-4 complex structural results were also used to try to understand why amino acid substitutions that were introduced into IL4-1359 during the affinity optimization process of IL4-1285 antibody contribute to the higher affinity for IL-4 resulting in increased potency of IL-4 neutralization. The anti-IL-4 antibody IL4-1285 forms a stable complex with its cognate ligand mainly though the N-terminal half of the IL-4 Helix C. While the overall interface between IL4-1285 and IL-4 is not extensive, CDRH3 is joined by CDRL1 and CDRL2 to cover the majority of exposed surface of Helix C. Based on the structural information of IL4-1285 antibody in complex with IL-4, CDRL3 is buried underneath CDRH3, and CDRH2 is distant from the Fab-ligand interface; neither can contribute to the IL4-1285 binding affinity to IL-4. However, CDRH1 is in close proximity of IL-4, but only interacts sparsely with the ligand. Consequently, optimization of binding through CDRH1 is more likely to make a significant contribution to the binding affinity with IL-4, as manifested in the optimized IL4-1359 variant. The following analyses reveal the underlying reasons for tighter binding of IL4-1359 to IL-4 upon affinity optimization of IL4-1285.

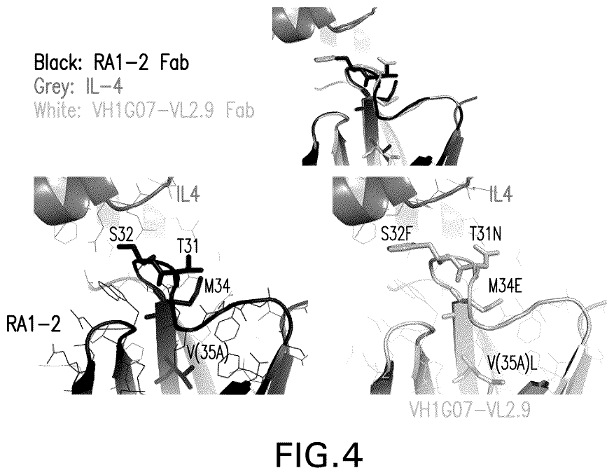

The IL4-1285/IL-4 complex structural results were also used to try to understand why changes that were introduced into IL4-1359 V_{H} during the affinity optimization process of IL4-1285 antibody contribute to the higher affinity for IL-4 resulting in increased potency of IL-4 neutralization. Using structure modeling of the IL4-1359 affinity optimized variant compared to the solved structure of the IL4-1285/IL-4 complex, the following changes that were incorporated into the IL4-1359 V_{H} CDRH1 relative to IL4-1285 that had the most impact for increasing affinity to IL-4 (FIG. 4):
1. T31N: Replacing Thr31 with an Asparagine makes it possible for Asn31 to form two additional salt bridges with Arg75 on Helix C of IL-4. These interactions are likely to improve binding affinity between IL-4 and IL4-1285 Fab.
2. Phenylalanine at position 32 in IL4-1359 V_{H} provides much better stabilizing interactions with the C-terminal end of the IL-4 Helix A, as well as its extending loop.
3. M34E: With T31N in place, the M34E difference ‘down-sizes’ the original methionine sidechain, affording more space to accommodate T31N. In addition, the M34E change optimizes the surface property of this residue so it may blend better in with the surrounding, which is more hydrophilic.
4. V(35A)L: The longer hydrophobic side of Leucine fills the void space inside the hydrophobic pocket surrounded by L4, F24, F27, V78, W36, C22, and C92. The V(35A)L change provides additional force to strengthen the interaction between two beta sheets of the Ig fold and strengthens the regional structure of the heavy chain.

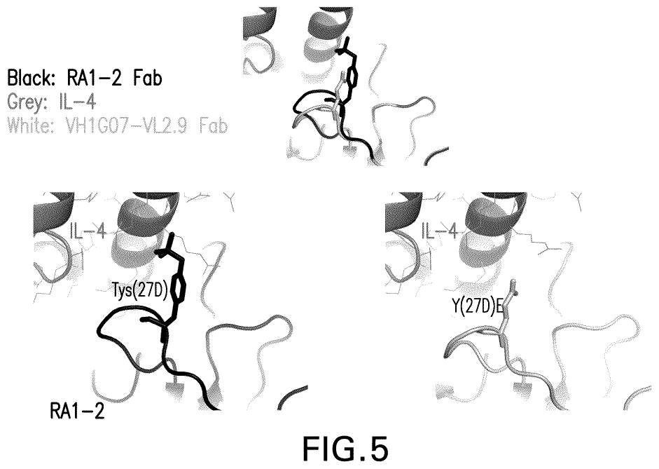

Biophysical characterization of IL4-1285 antibody revealed that there was an undesired post-translational modification (O-sulfation) occurring at Tyr residue L27d in IL4-1285 CDRL1 (KASQSVDYDGDSYMN, SEQ ID NO: 11). Using the IL4-1285 Fab/IL-4 complex structure results, protein structure modeling suggested that this Tyr residue could contribute to cytokine binding and led to the recommendation of using asparagine (IL4-1345 V_{L} or hu3B9-VL v2.7, V_{L}), phenylalanine (IL4-1346 V_{L} or hu3B9_VL v2.8 V_{L}) or glutamic acid (IL4-1347 V_{L} or hu3B9_VL v2.9 V_{L}, SEQ ID NO: 20) as a replacement for the tyrosine residue at L27d in the IL4-1285 VL v2.0. Additionally, the aspartic acid residue at L28 (Kabat numbering) was changed to glutamic acid (IL4-1348 V_{L}, hu3B9_VL v2.10 V_{L}) to evaluate degree of sulfation on Tyr (L27d). Results from structure modeling show that Y(L27d)E can also form salt bridge interaction with R81 of IL-4 and further stabilizes the ligand-IL4-1285 antibody interface and improve binding affinity (see FIG. 5).

### Example 14

### Determination of Humanized IL13-1307 (hu13.4) Antibody-Human IL-13 Complex Structure

To determine which residues in humanized IL13-1307 (hu13.4) antibody make a direct contact with human IL-13 cytokine and to enable structure-based library design, the structure was determined for the complex of IL13-1307 antibody with IL-13. Additionally, the structure for another humanized anti-IL-13 antibody, IL13-1283 (IMA-638 or hu13.2), in complex with human IL-13 was solved and compared to that with IL13-1307 (hu13.4) in order to understand why IL13-1307 antibody was marginally more potent than IL13-1283 (IMA-638) in the pSTAT6 Phosphorylation (method previously described in EXAMPLE 3 and EXAMPLE 10) and Monocyte CD23 Expression bioassays (method described in EXAMPLE 11) despite high sequence homology shared between these antibodies (Table 11).

TABLE 11
Bioactivity of IL13-1307 versus IL13-1283 in the pSTAT6
Phosphorylation and Monocyte CD23 Expression Bioassays

| | Neutralization of IL- | |
|---|---|---|
| | 13-induced pSTAT6 | Neutralization of IL- |
| | Phosphorylation | 13 Induced CD23 |
| Antibody | [pM] | Expression [pM] |
| IL13-1307 (hu13.4) | 224.6 | 213.9 |
| IL13-1283 (IMA-638) | 427.1 | 260.8 |

For this structure, human IL-13 was over-expressed in the E. coli cell line BL21 (DE3). Cells were lysed through micro-fluidizer in the presence of Complete Protease Inhibitors (Merck). After centrifuging at 25,000 g the inclusion bodies were washed and re-solubilized with 6 M Guanadinium-HCl (GuHCl), 20 mM DTT and 1 mM EDTA (pH 8.8). Sequential dialysis steps were performed to gradually remove GuHCl while introducing oxidized glutathione (to final concentration of 1 mM) to assist refolding. After complete removal of GuHCl, the refolded IL-13 was purified through HiTrap SP Sepharose FF, HiTrap Phenyl HP, and Superdex 75 16/60 (all three columns obtained through GE Healthcare) to obtain protein for structural studies. Next, Fab fragments of the IL13-1307 antibody were generated via digesting the antibody with immobilized Papain per manufacturer protocol (Thermo/Pierce), and subsequently purified through pre-packed Protein A Sepharose FF (GE Healthcare). Separation of the IL13-1307 antibody fragments were performed with a reverse pH gradient on Protein A Sepharose, under such an approach, the Fc was eluted from the column prior to Fab.

To form the complex, the IL13-1307 Fab and human IL-13 were mixed at a ratio of 1:1.2 with excess of ligand to drive complex formation. Final purification was performed using a Superdex 200 size-exclusion column (GE Healthcare). The complex was concentrated to 10.8 mg/mL for crystallization setup. Optimal crystals of the protein complex containing IL13-1307 Fab and human IL-13 were obtained in the following condition: 100 mM HEPES pH 7.0, 1000 mM Tri-sodium citrate. A large crystal of the complex diffracted to about 2.7 Å resolution. Crystals were transiently cryoprotected and synchrotron data collection was performed remotely at Advanced Photon Source (APS). Image frames were processed using software AutoPROC (Global Phasing Ltd). A complete dataset was obtained at 2.8 Å resolution. The data belongs to space group 1222, with unit cells as follows: a=65.184 Å, b=167.870 Å, c=245.172 Å, α=ß=γ=90°, with 2 complexes per asymmetric unit. Molecular Replacement searches using homology model of the previously reported IL13-1283 (IMA-638) Fab and human IL-13 structures yielded convincing solutions of each component. Refinement was performed using software BUSTER, and the final R/R_{free} factors at 2.8 Å are 0.1699 and 0.2362, respectively, with RMSD of bonds 0.01 Å, RMSD of angles 1.30°.

From the analysis of the structural results, the following residues in the IL13-1307 heavy chain are involved in direct contact with human IL-13: T28, S30, S31, Y32, A33, W47, S50, S52, S53, Y58, L95, D96, G97, Y98, Y99, F100 (Kabat numbering). The following residues in the IL13-1307 light chain are involved in direct contact with human IL-13: H(27D), Y49, R50, E55, N92, D94, W96. It was determined that the following residues in human IL-13 are involved in direct contact with IL13-1307 Fab: T2, A3, E6, L42, E43, 146, E55, K56, Q58, R59, M60, S62, G63, F64, C65, P66, H67, K68.

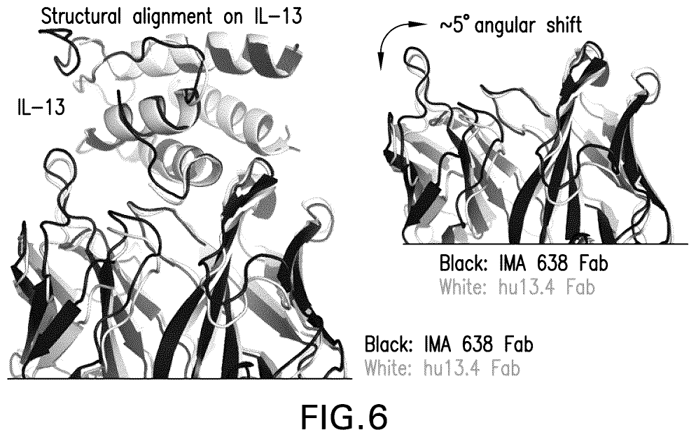

The IL13-1307 (hu13.4) antibody has slightly higher affinity toward its intended target IL-13 than IL13-1283 (IMA-638) resulting in increased potency in bioassays despite high sequence homology. There are seven residues in the CDR regions that differ between IL13-1283 and IL13-1307 located within the V_{H}: 130S, G55D, N56T, A101P and within the V_{L}: Y28S, K30S, N(27D)H. Due to these 7 amino acid differences, the interface between IL13-1307 Fab and IL-13 has a subtle but visible angular shift (˜5 degrees) from the interface between IL13-1283 Fab and IL-13 (FIG. 6), with a slightly more extensive interface (buried surface area of 2042.89 A2 for IL13-1307 Fab vs. 1982.5 A2 for IL13-1283 Fab). More importantly, there are five more residues in IL13-1307 Fab that involve an extensive interaction. In addition, there are more residues in IL13-1307 that involve salt-bridge interactions (seven in IL13-1307 Fab versus four in IL13-1283 Fab), which are substantially stronger than van der Waals' contacts.

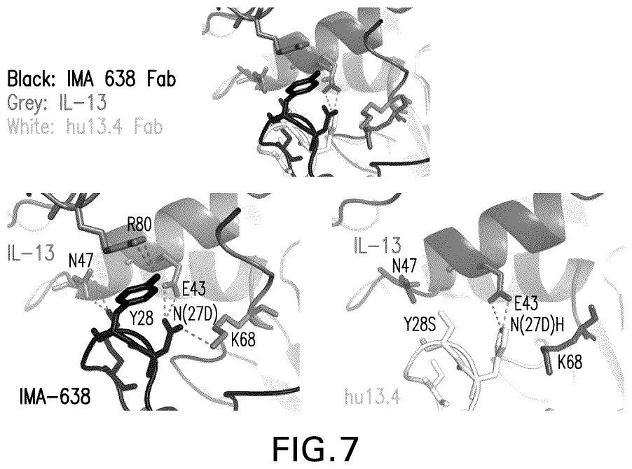

Y28 and K30 in the light chain of IL13-1283 Fab interact with R80 and N47 on the long loop between Helices C and D (residues 67-83) of human IL-13, respectively. Furthermore, N(27D) in the light chain of IL13-1283 Fab also interacts with K68 in the above referenced loop, as well as E43 on Helix B of human IL-13. Collectively, these interactions, as observed in the IL13-1283 Fab-IL13 complex structure, inevitably tilt the interface towards the peripheral side of human IL-13 and consequently weaken the interactions involving helices A & C of IL-13 and CDRH1 & 2 of IL13-1283 Fab. In IL13-1307 Fab, S28 and S30 do not involve any contact with IL-13, and H(27D) also loses its salt bridge contact with K68. In the absence of peripheral tethering effect, CDRH1 and CDRH2 of IL13-1307 Fab are able to engage more intimately with helices A and C of IL-13, and consequently result in better fit interface and stronger interactions (FIG. 7).

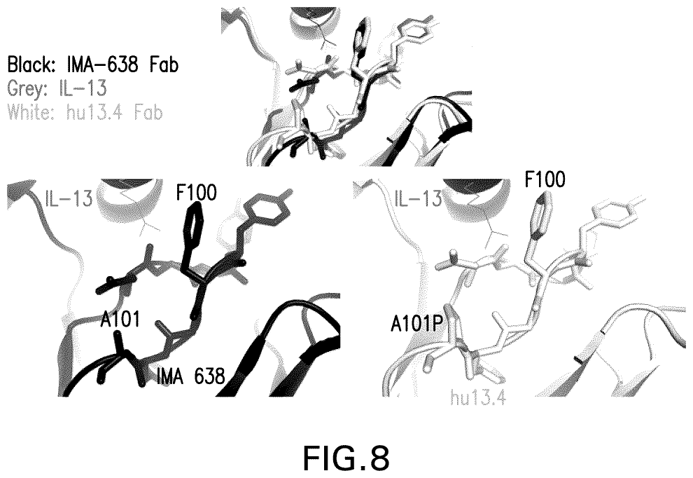

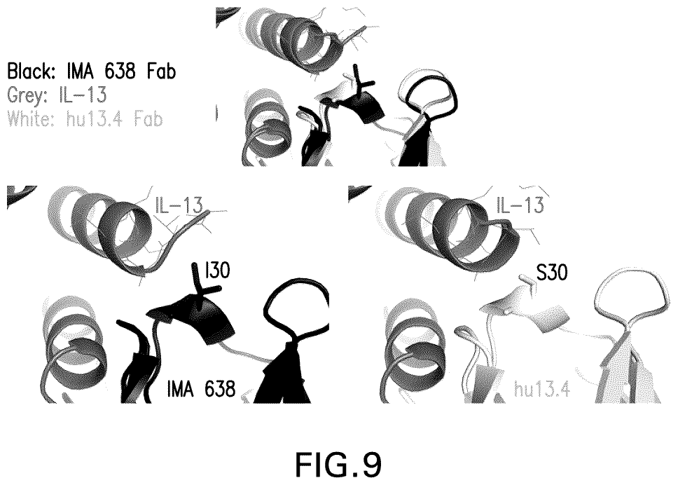

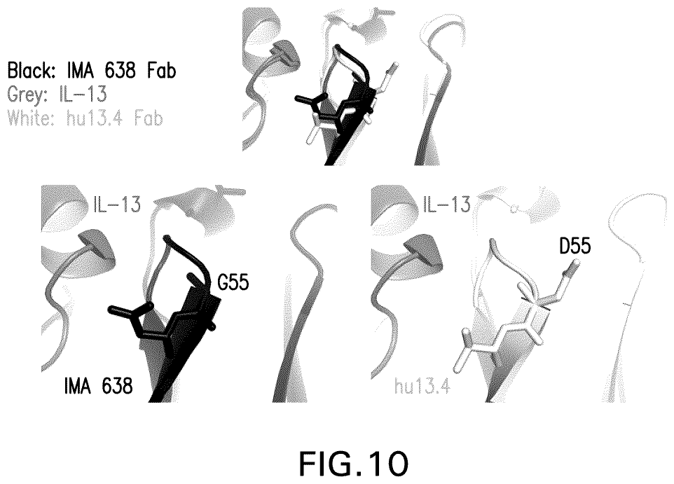

In addition to the improvement of ligand engagement as elaborated above, the following three amino acid difference in IL13-1307 Fab versus IL13-1283 also contribute synergistically that collectively result in higher binding affinity and observed increased potency in bioassays:
(1) The side chain of P101 (Alanine in hu13.2 Fab) fills in the void space next to D96 and provides additional structural re-enforcement of CDRH3 of IL13-1307 Fab, which interacts extensively with Helices B and C of IL-13. Consequently, residues such as F100 gains substantial interactions with IL-13 (FIG. 8).
(2) Serine (H30) of IL13-1307 Fab appears to be more compatible with the N-terminal region of IL-13, whose surface is hydrophilic in nature. Hence, S30 contained in IL13-1307 versus Isoleucine (H30) residue in IL13-1283 will likely contribute thermodynamically for the improved binding affinity (FIG. 9).
(3) The aspartic acid at position 55 in IL13-1307 Fab heavy chain versus the glycine at this position in IL13-1283, while does not involve interaction with IL-13 by itself, is likely to stabilize the conformation of CDRH2 for optimal engagement with Helix C of IL-13 (FIG. 10).

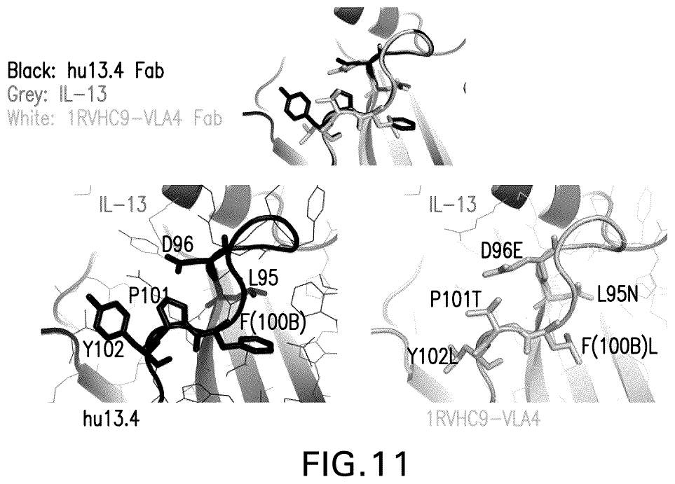

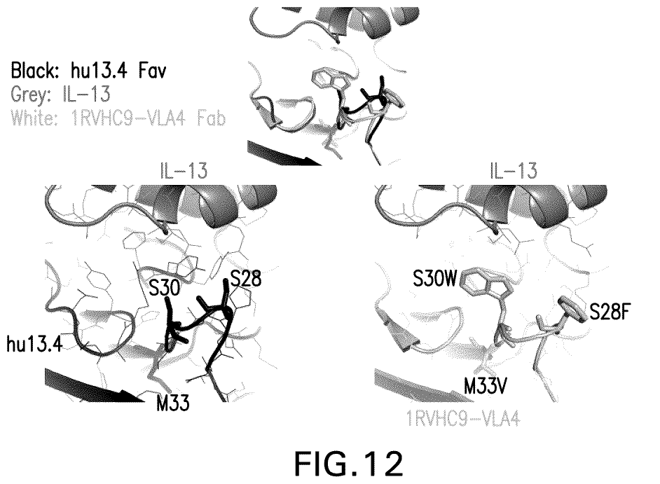

The structural results were also used to try to understand why mutations that were introduced into IL13-0001 (1RVHC9-VLA4) during the affinity optimization process of IL13-1307 antibody contribute to the higher affinity for IL-13 resulting in increased potency of IL-13 neutralization. Changes that were incorporated into the IL13-0001 V_{H} (SEQ ID NO: 51) CDRH3 relative to IL13-1307 that had the most impact for increasing affinity to IL-13 (see FIG. 11).
(1) L95N and F(100B)L: L95N and F(100B)L reduce hydrophobic anchoring interactions between CDRH3 and the remaining core region of mAb. Along with P101T, these three residues provide additional flexibility for CDRH3 to optimally engage IL-13.
(2) D96E: D96E extends the negatively charged residue closer to the mAb-ligand interface and interact with Arg59 of IL-13. Together with E55 in CDRL2, these two residues create a negatively charged surface to ensure stable interaction with Arg59 of IL-13.
(3) P101T: To accommodate D96E, P101T provides needed space for the extended sidechain of Glu and creates more hydrophilic environment, along with D96E and E55 of CDRL2, to attract Arg59 of IL-13.
(4) Y102L: Y102L provides better van der Waals' contact with Val2 of heavy chain N-terminal region and helps to provide further stability of the Ig fold.
   Similarly, amino acid differences that were incorporated into the VLA4 V_{L} (SEQ NO: 83) CDRL1 also contributed to increasing the affinity to IL-13 (see FIG. 12).
(1) S28F and S30W: S30W gains additional interaction with Asn47 of IL-13 via a hydrogen bond. Both are favored to mutate to bulky hydrophobic sidechains during phage display affinity optimization, likely to help attracting the exposed Trp29 of IL-13.
(2) The sidechain of M33 is tightly packed inside a small hydrophobic pocket with awkward conformation. The M33V change, with a smaller branched hydrophobic sidechain, provides better van der Waals' interactions with V27B and F71, and promotes local structural stability.

### Derivation of Anti-IL-33 Fab Binding Domain

### Example 15

### Removal of Deamidation Liabilities from IL-33 Binding Domain CDRL1

Derivation of neutralizing anti-IL-33 antibody IL33-0232 heavy and light chain variable regions, SEQ ID NO. 63 and SEQ ID NO. 68, respectively, was previously described (WO17187307). Mass spectrometric analyses (LC-MS/MS) were performed to assess thermal and pH stress on IL33-0232 antibody and evaluate level of each modification within the CDR regions at initial conditions (T=0) and stressed conditions (T=4w). For this analysis, IL33-0232 formulated into 20 mM Histidine, 85 mg/mL sucrose, 0.05 mg/mL EDTA, 0.2 mg/mL PS80, pH 5.8 (T=0) was buffer exchanged into: Tris pH 7.5, Histidine pH 5.8, Glutamic acid pH 4.5 buffers and then subjected to thermal stress (40° C.) for 4 weeks. Next, a low-artifact Lys-C/trypsin (LATD) peptide mapping LC-MS/MS method was performed at pH 6.0 and pH 8.2. The results identified a high level (>5%) deamidation hotspot at N³⁰ (KASQNIN³⁰KHLD: SEQ ID NO:65—underlined region shows peptide fragment of residues 25-31) and a low level (1-5%) deamidation hotspot at N²⁸ (KASQN²⁸INKHLD: SEQ ID NO:65; - underlined region shows peptide fragment of residues 25-31) within CDRL1 (Table 12).

To understand impact of complete (100%) conversion of Asparagine (Asn/N) to Aspartic acid (Asp/D) on ability to neutralize IL-33, the N³⁰ residue was substituted with an Asp residue to generate IL33-0216 antibody variant. Bioactivity of IL33-0216 antibody was determined using a NF-κB/AP-1-inducible secreted embryonic alkaline phosphatase (SEAP) reporter gene assay. For this IL-33 neutralization assay, HEK-Blue™ IL-33 Cells (Invivogen) are a HEK-293-based cell line engineered to lack TNF and IL-1 signaling and stably express both IL1 RL1 and a NF-κB/AP-1-inducible secreted embryonic alkaline phosphatase (SEAP) reporter gene. Upon IL-33 stimulation, these cells secrete SEAP, which can subsequently be quantitated using a colorimetric assay to assess activity of IL-33. HEK-Blue™ IL-33 Cells were maintained in DMEM (Gibco 11995-085) supplemented with 1× pen/strep/glu (Invitrogen 10378-016), 10% heat-inactivated FBS (Gibco 16140-171), 10 μg/ml blasticidin (Invitrogen A11139-03), 300 μg/mL zeocin (Invitrogen R25001) in a 37° C. incubator with 5% CO₂. Prior to assay, cells were released from maintenance flasks with trypLE (Gibco), washed, and resuspended at 10⁶ cells/mL. Cells were then seeded at 5×10⁴ cells/well in assay plates (Falcon 353072). A stock solution of recombinant human IL-33 (R&D Systems 3625-IL) at 100 μg/mL was diluted 1:100 by adding 1.5 μL of the IL-33 stock solution to 1.5 μL DTT (Sigma 646563) and 147 μL of media. DTT addition prevents redox-mediated inactivation of IL-33 which would prohibit downstream readouts. Alternatively, recombinant IL-33 mm2 Cys protein, a constitutively active mutant of IL-33, was used at a final concentration of 0.1 ng/mL. However this assay readout is less dynamic than that with reduced IL-33 protein. 25 μL of the anti-IL-33 antibody dilutions were added to the 50 μL of cells in each well followed by 25 μL of the diluted IL-33 mixture for a final concentration of 0.1 ng/mL of IL-33. Cells were stimulated for approximately 20 hours in a 37° C. incubator with 5% CO₂, at which time 75 μL of media was removed from each well of the culture plate for SEAP quantitation. 160 μL of QUANTI-Blue reagent (Invivogen) was added to each well of an assay plate (Falcon 353072). 40 μL of the cell-conditioned medium was added to each well and the plate was returned to 37° C. incubator for approximately 3 hours. SEAP activity was then assessed using a spectrophotometer (Spectramax M5e) at 650 nm. Antibody activity was assessed by the ability to suppress IL-33-induced SEAP activity.

Results from the IL-33 (R&D Systems 3625-IL) induced SEAP reporter gene bioassay that complete (100%) conversion of the Asn residue at position 30 (Pfabat numbering) within CDRL1 of IL33-0232 leads to a significant loss in IL-33 neutralization activity as indicated by significant loss of activity for IL33-0216 variant versus IL33-0232 (Table 13). This loss is even more apparent when comparing to the affinity optimized variant IL33-0726 harboring N(L30)H (Table 13). Derivation of IL33-0726 binding domain is described in EXAMPLE 16.

Efforts were undertaken to engineer IL33-0232 to remove both the high and low level deamidation sites at N(L30) and Q(L27), respectively. Selection of mutations to replace N(L30) and N(L28) were guided by computational and manual inspection of the binding interface in the co-crystal structure of the parental humanized 7E8 antibody with human IL-33. Using this structure guided approach, Ser (S), Gln (Q) and Tyr (Y) were identified as potential substitutions to replace N(L30). Additionally, one variant (IL33-0224) was engineered with N(L28)P and N(L30)S intended to remove both the high level and low level deamidation liabilities. The K(L24)R mutation was also incorporated into these variants since it removes an in silico predicted T-cell epitope and this amino acid substitution when incorporated into IL33-0232 resulting in variant IL33-0217 was shown to be tolerated and not alter IL-33 neutralization capability (Table 14). Evaluation of bioactivity for these variants were assessed in SEAP reporter gene assay induced with the IL-33 mm2 Cys constitutively active mutant protein. The results indicate that both Ser and Gln can be substituted for N(L30) to remove the high level deamidation liability and that the N(L30)S+N(L28)P combined mutations in IL33-0224 were tolerated and did not alter IL-33 neutralization activity (Table 14). Variant IL33-0224 was selected as the template for IL-33 affinity optimization since both high level and low level deamidation liabilities were removed (EXAMPLE 16).

TABLE 12
Quantification of Asparagine Deamidation Hotspots
Determined by LATD Peptide Mapping LC-MS/MS Method.

| KA²⁵SQNIN³⁰K³¹HLD Peptide | | | KA²⁵SQ²⁷NINK³¹HLD Peptide | | |
|---|---|---|---|---|---|
| (25-31 underlined portion of SEQ ID NO: 65) | | | (25-31 underlined portion of SEQ ID NO: 65) | | |
| LATD: His | pH 8.2 | pH 6.0 | LATD: His | pH 8.2 | pH 6.0 |
| % Asn (N) | 82.1 | 87.3 | % Asn (N) | 97.1 | 95.2 |
| % | 17.9 | 12.7 | % | 2.9 | 4.8 |
| Asp/isoAsp | | | Asp/isoAsp | | |
| Succinimide | ND | ND | Succinimide | ND | ND |
| formation | | | formation | | |

Asn = Asparagine; Asp = Aspartic acid; isoAsp = isoaspartate; ND = not detected.

TABLE 13
IL-33 Neutralization Activity of Variants of IL33-0232
Designed to Examine N(L30) Deamidation Liability.

| | | IC₅₀ [pM] versus human reduced | |
|---|---|---|---|
| | | IL-33 (R&D) | |
| Antibody | Description | Experiment 1 | Experiment 2 |
| IL33-0232 | N(L30) | 35 | ND |
| IL33-0216 | N(L30)D | *677 | *1235 |
| IL33-0726 | Affinity optimized variant with | ND | 6 |
| | N(L30)H | | |

*IL13433-0216 IC₅₀ values in both experiments are estimated since they did not reach complete inhibition; ND = Not determined.

TABLE 14
IL-33 Neutralization Activity of Variants of IL33-
0232 Designed to Remove N(L30) and N(L28) Deamidation

| | | IC₅₀ [pM] versus |
|---|---|---|
| | | human IL-33 mm2 |
| Antibody | CDRL1 Mutations | Cys |
| IL33-0216 | N(L30)D | 237 |
| IL33-0217 | K(L24)R | 158 |
| IL33-0219 | K(L24)R, N(L30)Q | 142 |
| IL33-0220 | K(L24)R, N(L30)Y | 176 |
| IL33-0224 | K(L24)R, N(L28)P, | 115 |
| | N(L30)S | |
| IL33-0232 | None | 142 |

### Example 16

### Increasing Antibody Binding Affinity and Neutralization Activity for IL-33 While Maintaining Low Non-Specificity Interactions

Dose prediction modeling was performed for the IL13433-0006 protype Trispecific molecule engineered with IL4-0002, IL13-0001 and IL33-0232 binding domains and either with or without the Leucine-Serine (LS) half-life extension mutations. The LS mutations (M(H459)L and N(H465)S (Pfabat numbering) were engineered into the Tri-Fab-Fc molecules to increase window of dosing flexibility. The dose prediction modeling suggested that potency of IL-33 neutralization for the IL33-0232 Fab would need to be increased 10-fold to allow for target coverage regardless of LS half-life extension mutations. The co-crystal structure of the parental humanized 7E8 antibody with human IL-33 shows that five CDRs interact with IL-33 and suggest that this complex interface would require parallel phage display approaches to identify variants with 10-fold improved affinity. IL33-0224 heavy and light chain variable regions, SED ID NO: 63 and SED ID NO: 71, respectively, were selected to engineer the parental single-chain variable fragment (scFv) template for phage display since both the high and low level deamidation liabilities were eliminated from CDRL1. As an agnostic approach, a random soft-mutagenesis phage display library was constructed which leaves ˜50% parental wild-type residue in the phage library at each targeted position, plus random amino acid content in the other 50%. A two round selection campaign was employed for each of the soft-mutagenesis sub-libraries, involving a stringent initial first round of selection against a range of biotinylated human IL-33: 200 pM, 100 pM, 10 pM, 1 pM with an incorporated overnight ‘off-rate’ challenge using 1000-fold excess of human IL-33. In the second round of selection, biotinylated human IL-33 was used at 200 pM, 100 pM, 10 pM, 1 pM with an ‘off-rate’ challenge of 100-fold excess human IL-33. Clones were picked from each selection branch and periplasmic preparations (peri-preps) containing scFvs were produced for screening to determine neutralization capability relative to the parental IL33-0224 and IL33-0232 scFvs using a Homogeneous Time Resolved Fluorescence (HTRF) assay. For this HTRF assay, 10 μL per well peri-prep was aliquoted into a 384-well plate using a JANUS® liquid handler (PerkinElmer). Then, 5 μL IL33-0232 IgG diluted to 4 nM was added per well to the 384-well plate along with anti-human IgG-Fc-europium diluted 1:100 for 1 nM and 1:400 final concentration and ratio, respectively. Next, biotinylated human IL-33, prediluted in 5 mM Dithiothreitol (DTT), was diluted to 4 nM and 5 μL was added to each well in a 384-well plate with a 1:750 dilution of Steptavidin (SA)-XL665 for 1 nM and 1:3000 final concentration and ratio, respectively. The HTRF samples were incubated within the 384-well plate for 3 hours at room temperature and time-resolved fluorescence (TRF) was measured using an EnVision Multimode Plate Reader (PerkinElmer).

A structure guided rational approach was also employed where the phage library was built with amino acids chosen at each CDR position based on computational and manual inspection of the 7E8 antibody/human IL-33 binding interface. A two round selection campaign was also used for the rational design sub-libraries including an extremely stringent initial first round of selection against a range of biotinylated human IL-33: 10 pM, 1 pM, 0.1 pM with an incorporated overnight ‘off-rate’ challenge using 1000-fold excess of human IL-33. In the second round of selection, biotinylated human IL-33 was used at 10 pM, 1 pM, 0.1 pM with an ‘off-rate’ challenge of 100-fold excess human IL-33. Clones were picked from each selection branch and peri-preps containing scFvs were produced for screening to determine neutralization capability relative to the parental IL33-0224 and IL33-0232 scFvs using the HTRF assay previously described for soft-mutagenesis phage display approach.

Hits identified from the soft mutagenesis and rational approaches were re-arrayed and activity was confirmed in the HTRF assay. Confirmed hits were sequenced to determine uniqueness of the V_{H} and V_{L}. Unique hits were converted to an IgG format and protein was produced via transient HEK-293 expression system. Select V_{H} and V_{L} isolated from the soft mutagenesis approach that were reformatted into heavy and light chain expression vectors, respectively, were co-transfected to increase the diversity of clones and identify if certain combinations would yield synergistic increase in binding affinity to IL-33. Resultant IgG protein was evaluated for ability to neutralize IL-33 in the NF-κB/AP-1-inducible SEAP reporter gene assay (EXAMPLE 15) relative to IL33-0232 and IL33-0352 (an antibody with the variable regions of the IL-33 antibody Itepekimab (REGN3500; Regeneron) grafted to human IgG1 effector function null constant regions in place of the original human IgG4—see SEQ ID NO: 274 and SEQ ID NO: 282 of US20140271658), and assessed using a set of in vitro assays that examine various physicochemical properties of antibodies that may lead to unfavorable and favorable Pharmacokinetics (PK). Assessment in the suite of in vitro non-specificity assays (AC-SINS, DNA and Insulin binding ELISAs) was incorporated into the screening triage to identify the IL-33 affinity optimized candidate since the 7E8 antibody exhibited elevated non-specificity scores due to the charge-based interaction with IL-33.

The AC-SINS assay is a high-throughput method for detection of antibodies with a propensity to self-associate and utilizes the optical properties of gold nanoparticles. Briefly, antibodies are captured by anti-human Fc antibodies coated on the gold nanoparticles and if an antibody tends to interact with itself, there is a clustering of the nanoparticles, which leads to a red shift in the absorbance wavelength. This assay has also been described in the literature as a potential screening tool for identifying developability issues related to solubility, viscosity, and aggregation. For the AC-SINS assay, 20 nm gold nanoparticles (Ted Pella, Inc., catalog #15705) were coated with a mixture of 80% goat anti-human Fc (Jackson ImmunoResearch Laboratories, Inc. catalog #109-005-098) and 20% non-specific goat polyclonal antibodies (Jackson ImmunoResearch Laboratories, Inc. catalog #005-000-003) that were buffer exchanged into 20 mM sodium acetate pH 4.3 and diluted to 0.4 mg/mL. The plate was incubated for 1 hour at room temperature. Next, unoccupied sites on the gold nanoparticles were blocked with thiolated polyethylene glycol (2 kD). The coated/blocked nanoparticles were then concentrated 10-fold using a syringe filter and 10 μL were added to 100 μL of the sample (Tri-Fab-Fc or antibody) at 0.05 mg/mL in PBS pH 7.2. The coated/blocked nanoparticles were incubated with the sample of interest for 2 hours in a 96-well polypropylene plate, transferred to a 384-well polystyrene plate and the absorbance was read on a Tecan M1000 spectrophotometer from 450-650 in 2 nm increments. A Microsoft Excel macro was used to identify the max absorbance, and smooth and fit the data using a second-order polynomial. The smoothed maximum absorbance of the average blank (PBS buffer alone) was subtracted from the smoothed maximum absorbance of the sample to determine the AC-SINS score. Ranking of AC-SINS scores is the following: Good 0-5, Moderate >5 and <10, High >10.

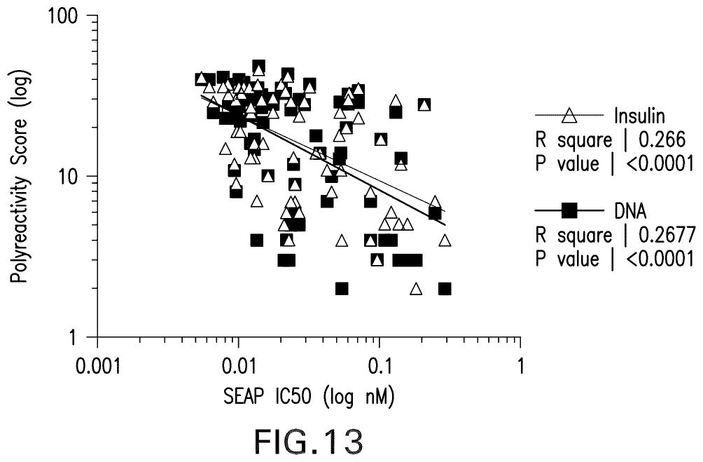

DNA- and Insulin-binding ELISAs were employed to measure low-affinity charge-based interactions of antibodies as a measure of polyreactivity using the following method adapted from Tiller et al. with a Janus Automated Workstation liquid handling robot (PerkinElmer). For these assays, DNA (Sigma-Aldrich, D1626) or Insulin (Sigma-Aldrich, 19278-5 mL) diluted in PBS-CMF pH 7.2 to 10 μg/mL or 5 μg/mL, respectively, was pipetted into 384-well ELISA plates (Nunc Maxisorp) and incubated at 4° C. overnight. The plates were then washed with water, blocked with 50 μl of Polyreactivity ELISA Buffer (PBS containing 0.05% Tween-20, 1 mM EDTA) for 1 hour at room temperature, and rinsed three times with water. Serially diluted samples were added in quadruplicate to the wells and incubated for 1 hour at room temperature. Plates were washed three times with water, and goat anti-human IgG conjugated to horseradish peroxidase (Jackson ImmunoResearch, 109-035-008) diluted to 10 ng/mL was added to plates and incubated for 1 hour at room temperature. Next, the plates were washed three times with water and then TMB substrate (Sigma-Aldrich, T-0440) was added to plates. Reactions were stopped after approximately 7 minutes by adding 0.18 M ortho-phosphoric acid to each well and absorbance read at 450 nm. The DNA and insulin binding scores were calculated as the ratio of the ELISA signal of the sample (Tri-Fab-Fc or antibody) at 10 μg/mL versus the signal of control wells containing buffer instead of the primary antibody. Ranking of polyreactivity scores are the following: Good 0-5, Moderate >5 and <10, High >10. Multiple affinity optimized clones were isolated from the soft mutagenesis approach and sequencing of top ranked scFvs revealed 38 V_{L} and 32 V_{H} unique hits. The top ranked scFvs that were re-formatted to IgG also exhibited a significant increase in potency of human IL-33 neutralization that was determined using the NF-κB/AP-1-inducible SEAP reporter gene assay and showing 4-10 fold range of improvement relative to IL33-0232 (Table 15). Bioactivity of these variants was also comparable or slightly improved relative to the clinical benchmark antibody IL33-0352. However as anticipated, the increase in IL-33 neutralization ability correlated with a notable increase in non-specificity scores (FIG. 13 and Table 15). Multiple improved clones were identified from the rational design approach, 176 scFv clones were identified from the primary HTRF screen and 102 clones selected from the confirmation screen for sequencing. The top ranked scFv variants that were re-formatted to IgG also presented a substantial increase in potency of human IL-33 neutralization that was ascertained using the NF-κB/AP-1-inducible SEAP reporter gene bioassay with a 4-16 fold range of improvement relative to IL33-0232 (Table 16). Bioactivity of these variants was also comparable or slightly improved relative to the clinical benchmark antibody IL33-0352 (Table 16). Similar to the observation with soft mutagenesis phage display derived variants, increase in potency of IL-33 neutralization in the bioassay correlates with a significant increase in non-specificity scores relative to IL33-0232 (Table 16). IL33-0721 and IL33-0726 exhibited the best balance of potency and non-specificity scores of clones screened from both soft mutagenesis and rational design approaches (Table 16).

Kinetics Exclusion Assay (KinExA) solution affinity was performed to evaluate binding to human IL-33 for the affinity optimized IL33-0726 IgG relative to the parental IL33-0232 IgG and the IL33-0352 benchmark antibody. Samples were prepared in PBS containing 0.1% sodium azide and 1.0 mg/mL bovine serum albumin (BSA). Affinity determinations using the fixed antigen assay format were performed by titrating the antibodies in a two-fold dilution series ranging from 122 fM to 1000 pM and 244 fM to 2000 pM 244 fM into a fixed concentration of biotinylated human IL 33. The fixed active binding concentrations (ABC) of biotinylated human IL 33 used were 10 pM and 100 pM. Biotinylated human IL-33 wild-type (WT) was reduced with 3 mM Dithiothreitol (DTT) for 2 hours at room temperature before use. The samples were allowed to equilibrate for at least 72 hours at room temperature before passing them through a flow cell that contained the test article anti-IL-33 antibody being evaluated adsorbed to polymethylmethacrylate (PMMA) beads (Sapidyne). The free biotinylated IL-33 cytokine captured with the test article anti-IL-33 antibody being evaluated was detected with 0.5 μg/mL Alexa Fluor 647-conjugated streptavidin (Jackson Immunoresearch). Data analysis was performed with KinExA Pro software version 4.3.11 (Sapidyne). The ‘affinity standard’ model was used to analyze the data and determine the K_{D} and active concentration of the IL-33 cytokine. The ‘drift correction’ fitting option was used when responses varied between replicate injections. Two curves were obtained in independent experiments and analyzed using the ‘n-curve analysis’ tool to obtain global best fit values for the K_{D} and active concentration of IL-33 cytokine. The software reports each best fit value along with a 95% confidence interval. The results show that the bivalent IL33-0726 IgG affinity appears to be below the KinExA limits of detection, so it is projected to have ˜14-fold higher affinity than IL33-0232 antibody (<236.88 fM versus 3.4 pM) and ˜3-fold higher affinity relative to the IL33-0352 benchmark antibody (<236.88 fM versus 659.99 fM, Table 17). Additionally, human IL-33 WT KinExA affinity measurements were determined to be 106.10 fM for IL13433-1258 Tri-Fab-Fc which harbors the monovalent IL33-0726 Fab binding domain and thus presents ˜32- and ˜6-fold increased affinity versus the IL33-0232 IgG and IL33-0352 benchmark antibody, respectively (Table 17). IL13433-1270 Tri-Fab-Fc that was also engineered with the IL33-0726 Fab binding domain has ˜4-fold increased affinity versus the IL33-0232 IgG and similar to the IL33-0352 clinical benchmark antibody (Table 17).

TABLE 15
Potency of IL-33 Neutralization and Non-specificity
Scores for Lead Anti-IL-33 Antibody Variants
Isolated from Soft Mutagenesis Phage Approach.

| | AC-SINS | | | IC₅₀ [nM] | | |
|---|---|---|---|---|---|---|
| | Δ Max | Polyreactivity | | versus reduced | | |
| | Absorbance | Scores | | human IL-33 (R&D) | | |
| Antibody | (Mean) | DNA | Insulin | IC₅₀ (AVE) | SDM | n |
| IL33-0232 | 3 | 6 | 5 | 0.071 | 0.013 | 3 |
| IL33-0352 | 8 | 13 | 14 | 0.018 | 0.005 | 2 |
| (Clinical | | | | | | |
| Benchmark) | | | | | | |
| IL33-0698 | 17 | 45 | 20 | 0.014 | 0.002 | 4 |
| IL33-0701 | 17 | 45 | 15 | 0.013 | 0.002 | 4 |
| IL33-0702 | 19 | 49 | 19 | 0.008 | 0.002 | 4 |
| IL33-0704 | 16 | 30 | 28 | 0.015 | 0.006 | 2 |
| IL33-0705 | 13 | 22 | 32 | 0.007 | 0.000 | 2 |
| IL33-0706 | 7 | 32 | 32 | 0.018 | 0.005 | 2 |
| IL33-0707 | 15 | 35 | 36 | 0.014 | 0.000 | 2 |
| IL33-0710 | 19 | 32 | 22 | 0.013 | 0.000 | 2 |
| IL33-0711 | 21 | 32 | 29 | 0.007 | 0.000 | 2 |
| IL33-0712 | 19 | 35 | 22 | 0.010 | 0.000 | 2 |
| IL33-0713 | 19 | 28 | 20 | 0.012 | 0.002 | 2 |
| IL33-0714 | 20 | 42 | 31 | 0.009 | 0.002 | 2 |
| IL33-0715 | 17 | 39 | 27 | 0.012 | 0.004 | 2 |

TABLE 16
Potency of IL-33 Neutralization and Non-specificity
Scores for Lead Anti-IL-33 Antibody Variants
Isolated from Rational Design Phage Approach.

| | AC-SINS | | | IC₅₀ [nM] | | |
|---|---|---|---|---|---|---|
| | Δ Max | Polyreactivity | | versus reduced | | |
| | Absorbance | Scores | | human IL-33 (R&D) | | |
| Antibody | (Mean) | DNA | Insulin | IC₅₀ (AVE) | SDM | n |
| IL33-0232 | 3 | 3 | 5 | 0.109 | 0.049 | 2 |
| IL33-0352 | 7 | 10 | 16 | 0.011 | 0.003 | 2 |
| (Clinical | | | | | | |
| Benchmark) | | | | | | |
| IL33-0718 | 15 | 29 | 28 | 0.008 | 0.002 | 3 |
| IL33-0719 | 11 | 20 | 14 | 0.020 | 0.006 | 3 |
| IL33-0721 | 17 | 3 | 7 | 0.026 | 0.004 | 3 |
| IL33-0722 | 14 | 15 | 16 | 0.015 | 0.003 | 3 |
| IL33-0723 | 10 | 16 | 13 | 0.017 | 0.005 | 3 |
| IL33-0724 | 17 | 16 | 23 | 0.015 | 0.002 | 3 |
| IL33-0726 | 5 | 15 | 13 | 0.008 | 0 | 3 |
| IL33-0727 | 15 | 22 | 15 | 0.017 | 0.002 | 3 |
| IL33-0728 | 14 | 21 | 17 | 0.016 | 0.002 | 3 |
| IL33-0737 | 15 | 38 | 44 | 0.007 | 0.002 | 2 |
| IL33-0738 | 16 | 29 | 24 | 0.007 | 0.002 | 2 |
| IL33-0739 | 17 | 29 | 27 | 0.010 | 0.000 | 2 |
| IL33-0740 | 16 | 27 | 22 | 0.012 | 0.004 | 2 |
| IL33-0741 | 16 | 33 | 33 | 0.016 | 0.010 | 2 |

TABLE 17
Affinity Determined for IgG Variants and Tri-Fab-Fc Molecules
Harboring IL33-0726 Binding Domain to Human IL-33 using
KinExA Solution Affinity Fixed Antigen Assay Format.

| Sample | Reference Constant | Global Fit KinExA K_{D} [fM] |
|---|---|---|
| IL33-0726 IgG | Human IL-33 | <236.88 |
| IL33-0232 IgG | Human IL-33 | 3400 |
| IL33-0352 IgG (Clinical Benchmark) | Human IL-33 | 659.99 |
| IL13433-1258 Tri-Fab-Fc | Human IL-33 | 106.10 |
| IL13433-1270 Tri-Fab-Fc | Human IL-33 | 753.31 |

### Derivation of TSLP Binding Domain

### Example 17

### Framework Grafting and Generation of Variants for Anti-TSLP Domain

Anti-TSLP antibody Tezepelumab is an IgG2/lambda (3) with human frameworks (FVV) IGVH3-33 and IGLV3-21*02. Co-crystal structure results had revealed binding paratopes of tezepelumab to TSLP (4). In addition to the binding sites from HCDRs, methionine at position 2 of HFW1 (valine at germline) and germline arginine at position 94 of HFW3 contributed to TSLP binding as well. The VH CDR1 (SEQ ID NO: 82), VH CDR2 (SEQ ID NO: 83) and VH CDR3 (SEQ ID NO: 84) were grafted onto various frameworks and expressed in combination with the various light chain frameworks bearing CDRs, VL CDR1 (SEQ ID NO: 86), VL CDR2 (SEQ ID NO: 87), and VL CDR3 (SEQ ID NO: 89). The cDNAs containing human acceptor frameworks with relevant CDR donor sequences were synthesized from Blue Heron Biotech. Synthesized cDNA products were subcloned and fused in frame with human IgG1 constant region with effector function null mutation (EFN, Pfabat number: L247A, L248 Å, G250A; EU number L234A L235 Å and G237A) (SEQ ID NOs: 6, 7, 8, 9) for the heavy chain, and human lambda for the light chain (SEQ ID NO: 95) in mammalian expression vectors.

### Example 18

### Primary Screening of Anti-TSLP Framework (FW) Engineered Variants

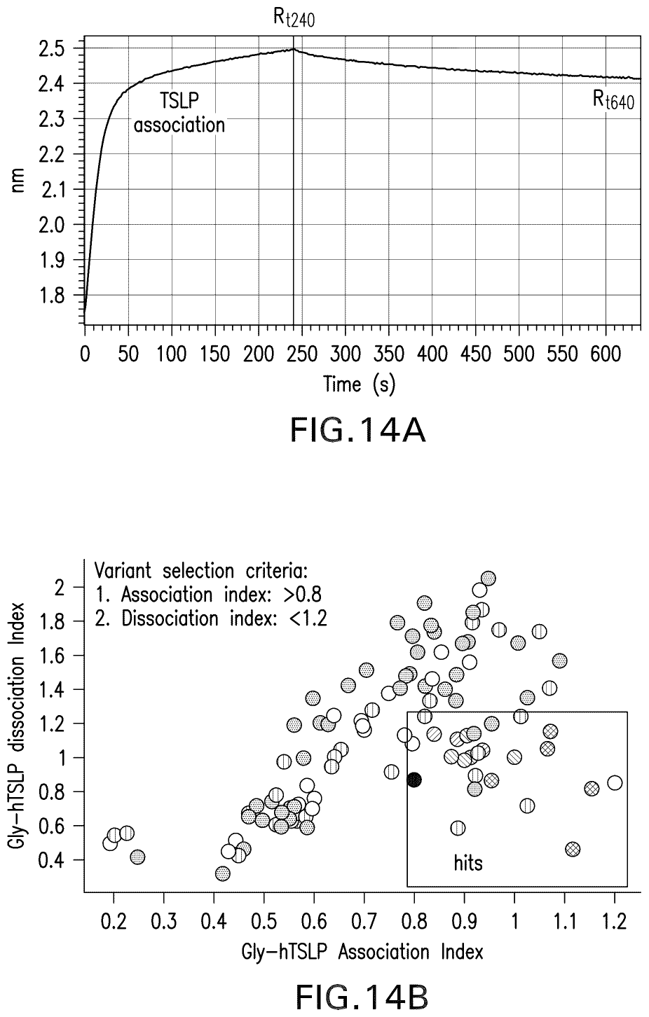

All variants were generated as IgG molecules using standard expression and purification techniques well known in the art and evaluated through high throughput Octet off-rate screening (FIG. 14a), AC-SINs (Affinity-Capture Self-Interaction Nanoparticle Spectroscopy, to measure self-association propensity of mAbs), DNA and insulin polyreactivity (DNA and insulin binding ELISA to measure antibody non-specific polyreactivity) as primary screening methods.

The high throughput off-rate screening assay by OctetRed (ForteBio) was designed to evaluate binding activity of the variants to glycosylated TSLP (IfhTSLP-avi-v5-his6) in comparison to TSLP-0001 antibody ((SEQ ID NO: 484 (HC) and 488 (LC)). All reagents were mapped out in a black flat bottom 384-well plate (Fortebio, Cat #18-5080) and 16-biosensor mode was used to speed up the screening process. Anti-human IgG Kinetics biosensors (Fortebio) were used to capture the anti-TSLP antibodies at a concentration of 20 ug/ml for 180 s, followed by a baseline in PBS for 60 s, then dipped into 5-10 ug/ml of soluble human TSLP antigen 240 s for association, and finally dissociated in PBS for 400 s. Sensorgrams were examined to determine whether the antigen bound to the antibody and the Octet Data Analysis 8.1 software was used to determine Response (R, nm) at specified time points. The association index and dissociation index were calculated. Association index=Sample (Rt₂₄₀−Rt₀)/TSLP-0001 (Rt₂₄₀−Rt₀). Dissociation index=Sample (Rt₆₄₀−Rt₂₄₀)/TSLP-0001 (Rt₆₄₀−Rt₂₄₀). Rt₀ is the Response value when association starts (time zero). Rt₂₄₀ is the Response value at time 240 seconds (when association ends, and dissociation starts). Rt₆₄₀ is the Response value at time 640 seconds (when dissociation ends). A positive control TSLP-0001 and a negative control mab8.8 were included in each set of 16 biosensors. The value of TSLP-0001 (Rt₂₄₀-Rt₀) used for each testing sample was from the same set of 16 biosensor. The variants were selected for next round of screening if association index is >0.8 and dissociation index is <1.2. Screening results are shown at FIG. 14b. The variants were also screened by AC-SINs and DNA/Insulin polyreactivity assay (7). Scores of less than 10 represented acceptable self-association and polyreactivity.

The binding ELISAs for DNA and insulin score assay used a low-stringency protocol originally developed for detection of low-affinity autoantibodies from lupus patients. In brief, insulin at 5 μg/ml or single-stranded or double-stranded DNA at 10 μg/ml in PBS were coated onto 384-well Nunc Maxisorp ELISA plates overnight. Wells were washed 3× with water, then blocked with ELISA buffer (PBS/0.05% Tween/1 mM EDTA) 1 hour at room temperature. 25 ul of serially diluted mAbs in ELISA buffer were added to the plates and incubated for 1 hour at room temperature, and the wells were washed 3× with water, incubated with HRP-conjugated goat anti-human IgG 1:5000 in ELISA buffer for 1 hour at room temperature. Following 3 washes with water, color was developed with BioFX TMB (BioFX Laboratories, catalog #TMBW-0100-01) for 5 mins and the reaction stopped with 0.1M sulfuric acid.

AC-SINS assay was standardized in a 384-well format on a Perkin-Elmer Janus liquid handling robot. Testing antibodies were captured by 20 nm gold nanoparticles (Ted Pella, Inc., Cat #15705) coated with a mixture of 80% goat anti-human Fc (Jackson ImmunoResearch Laboratories, Inc. Cat #109-005-098) and 20% non-specific goat polyclonal antibodies (Jackson ImmunoResearch Laboratories, Inc. Cat #005-000-003). The absorbance was read from 450-650 in 2 nm increments, and a Microsoft Excel macro was used to identify the max absorbance, smooth the data, and fit the data using a second-order polynomial. The smoothed max absorbance of the average blank (PBS buffer alone) was subtracted from the smoothed max absorbance of the antibody sample to determine the antibody AC-SINS score.

The hits that passed through primary screening criteria were taken forward for competition ELISA assay. The competition ELISA was carried out to assess whether humanized variants can compete with TSLP-0001 for TSLP binding. Recombinant TSLP was coated onto 384-well Maxisorp plates (NUNC) at 25 ul of 1 μg/mL in PBS overnight at 4° C. Plates were then blocked (PBS with 3% BSA) and washed (PBS with 0.02% tween20) following standard ELISA protocol. A 4-fold serial diluted variants or negative control antibody mab8.8 were mixed with constant level (69 pM) of biotinylated TSLP-0001. 25 μL of such mixture was then added to the antigen coated plate and incubated for 1 hour at room temperature. After washing out the unbound, the amount of bound biotin-TSLP-0001 was detected by HRP conjugated streptavidin (Southern biotech, Cat #7100-05). Color was developed with BioFX TMB (BioFX Laboratories, Cat #TMBW-0100-01) for 5 mins and the reaction stopped with 0.1M sulfuric acid.

Four conventional bivalent IgG hits show comparable IC₅₀ as TSLP-0001 (Table 18). Among the four variants, 3 out of 4 are in IGVH3-33 FW while TSLP-0104 is in IGVH3-21*02 FW.

TABLE 18
IC₅₀ of anti-TSLP variants by competition ELISA

| | | Competition ELISA |
|---|---|---|
| Antibody Name | Sample ID | IC₅₀ (nM) |
| TSLP-0001 | TSLP-0001 | 0.599 |
| TSLP-0010 | IL413TSLP-0010 | 0.88 |
| TSLP-0015 | IL413TSLP-0015 | 0.53 |
| TSLP-0100 | IL413TSLP-0100 | 0.87 |
| TSLP-0104 | IL413TSLP-0104 | 0.88 |

### Example 19

### Characterization of FW Engineered Lead TSLP-0100

The TSLP neutralizing bioassay examines antibody inhibition of release of the chemokine TARC induced by TSLP stimulated monocytes. Primary human monocytes were enriched from human whole blood using the RosetteSep monocyte kit (Stemcell, #15068) and seeded in 96-well flat-bottom plates (Falcon, #353072) at 0.5×10⁶/mL in media with 150 uL/well. Dilutions of glycosylated long form human TSLP and mAb were added to bring the total volume to 200 uL. Plates were incubated 24 hours at 37° C. Supernatants were collected and assayed for TARC concentration using the human TARC MSD 96-well VPLEX (V-PLEX Human TARC Kit (MESO SCALE DIAGNOSTICS LLC, K151NTD-2)). Data at Table 19 show that IL134TSLP-0100 maintains similar TSLP neutralizing activity as parental TSLP-0001 (Table 19) and was selected as framework engineered lead and was used as template for affinity improving engineering.

Differential Scanning Calorimetry (DSC, 5, 6) was used to assess thermal stability of IL13TSLP-0100. Samples at 0.3 mg/mL were dispensed into the sample tray of a MicroCal VP-Capillary DSC with Autosampler (Malvern Instruments, Inc.), equilibrated for 5 mins at 10° C. and then scanned up to 110° C. at a rate of 100° C. per hr. A filtering period of 16 secs was selected. Raw data was baseline corrected and the protein concentration was normalized. Origin Software 7.0 (OriginLab Corporation, Northampton, MA) was used to fit the data to an MN2-State Model with an appropriate number of transitions. Data was shown at Table 19. IL4123TSLP-0100 shows improved thermal stability over TSLP-0001 antibody.

Low pH hold experiment was carried out to assess stability of the antibodies at pH3.4. 50 ug of antibody at 1.5 mg/mL concentration was acidified to pH3.4 (using 5 μL 0.04M glycine, pH 2.8) and incubated at room temperature for 5 hours. Percent of high molecular weight species (% HMMS) were analyzed by analytic SEC after neutralization to pH7.2. Same antibody at PBS pH7.2 was used as control. Delta % HMMS ((Δ% HMMS (Sample-Control)) was calculated to assess the stability. Data is shown at Table 19. IL4123TSLP-0100 improved low pH hold stability over TSLP-0001 antibody.

iCE (imaged capillary electrophoresis) was carried out to evaluate charge heterogeneity under high voltage and detection at absorbance 280 nm. Carrier ampholytes produce a pH gradient and proteins migrate until their net charge is zero. Electropherograms are analyzed to determine pI values and peak areas for acidic, main, and basic species. Protein Simple iCE3 instrument with PrinCE Autosampler were used to analyze samples. Proteins were diluted to 2 mg/mL in water. Sample diluent contained 0.01 mg/mL pI marker 4.65, 0.01 mg/mL pI marker 9.5, 4.0% Pharmalyte pH 3-10, 0.25% methyl cellulose, and 2.0 M urea. Samples contained 15 μL protein at 2 mg/mL and 85 μL sample diluent. Samples were focused for 1 minute at 1500 Volts and then 6 min at 3000 Volts. Data is shown at Table 19. IL4123TSLP-0100 reduced acidic species over TSLP-0001 antibody.

TSLP-0100 was comprised TSLP-0001 parental variable heavy chain but with germlined T70S and N79Y at HFW3 and parental light chain. Sequence comparison between TSLP-0100 and parental TSLP-0001 suggests that T70S and N79Y amino acid changes are responsible for the biophysical property improvement. The polyreactivity and AC-SINs scores remain similar to parental TSLP-0001 antibody (Table 19)

TABLE 19
Characterization of TSLP-0100

| | | | a-TSLP | | | | T0 iCE | | | |
|---|---|---|---|---|---|---|---|---|---|---|
| | | | bioassay | | | low pH3.4 | (% | | | |
| Antibody | VH | VL | (IC₅₀, | Tm | | hold | acidic | AC- | DNA | Insulin |
| Name | mutation | mutation | pM) | Fab(° C.) | Tm1(° C.) | Δ%HMMS | species) | SINs | score | score |
| TSLP- | VH- | VL-0001 | 19.2 ± 1.55 | 66.8 | 66.7 | 7.3 | 41.2 | 0 | 2 | 2 |
| 0001 | 0001 | | | | | | | | | |
| TSLP- | T70S, N79Y, | VL-0001 | 24.6 ± 3.97 | 69.9 | 69.1 | 0.2 | 27.1 | 1 | 3 | 2 |
| 0100 | SEQ | | | | | | | | | |

Immunogenicity was analyzed by Epivax and IEDB for in silico prediction of potential T-cell Epitopes. Amino acid sequences were analyzed for EpiMatrix analysis using ISPRI software (ISPRI v 1.8.0, EpiVax Inc., Providence, RI; 26), which provides rankings of likelihood of binding of each 9-mer amino acid fragment against 8 different HLA types. A hit is defined as those with Z-scores in the top 5% and a strong hit in the top 1%. Hits to 4 or more alleles or 1 strong hit are being considered as predicted t-cell epitopes. A second method analyzed sequences using the MHC-II binding Consensus method (27) in IEDB (IEDB MHC-II Binding Predictions), which provides ranking of likelihood of binding of 9-mers and 15-mers against 8 HLA types. Each epitope determined by these methods is classified as a germline or non-germline epitope, then further classified based on its location within the antibody (CDR or non-CDR). Score of less than −50 is acceptable. In silico immunogenicity prediction analysis show that 2 potential non-germline T-cell epitopes were eliminated through N79Y germling and improved overall immunogenicity score (Table 20).

TABLE 20
Immunogenicity analysis of TSLP-0001 and TSLP-0100

| | In silico | 9-mer of | AA | Position |
|---|---|---|---|---|
| Antibody | Immunogenicity | Predicted t-cell | position | number in |
| Name | score (overall) | epitope (VH) | (Pfabat) | SEQ ID NO: 91 |
| TSLP-0001 | −55.61 | LNLQMNSLR- | H78-H86 | 79-87 |
| | | NLQMNSLRA- | H79-H87 | 80-88 |
| | | VYYCARAPQ | H89-H97 | 93-101 |
| TSLP-0100 | −70.83 | - | H89-H97 | 93-101 |
| | | VYYCARAPQ | | |
| | | - | | |

Note:
AA: amino acid

### Example 20

### Engineering Strategies to Improve Anti-TSLP Activity

A comprehensive engineering strategy were implemented, including co-crystal structure-based rational mutagenesis and phage display (scFv library). FW engineered TSLP 0100 was used as a template. Computational modeling was used to identify mutations predicted to either increase the affinity of the antibody to TSLP or that would add a new interaction between the antibody and the TSLP domain, while at the same time being tolerated without the loss of stability and solubility (13). The method utilized in these calculations used an x-ray crystal structure from the Protein Databank (www.rcsb.org), PDB ID: 5J13, of the complex of TSLP with an anti-TSLP antibody (TSLP-0001).

The predicted stability and affinity were determined by two applications, Discovery Studio and Fold X (14, 15). For the Discovery Studio calculations, the initial PDB formatted structures were converted to a .dsv formatted structures by applying the “Prepare Protein” protocol of Discovery Studio 4.5 (Accelrys Inc.). The change in binding affinity upon mutation was calculated by applying the “Calculate Mutation Energy (Binding)” protocol from Discovery Studio 4.5 using a B-C-A chain dsv formatted structure. All point mutants in all 6 CDRs were explored. In addition to affinity, change in stability of the Fab upon mutation were calculated using the B-C chain dsv file. Here we applied the Calculate Mutation Energy (Stability)” protocol from Discovery Studio 4.5. From this set of predictions, we identified a number of target sites and mutations that could potentially increase the affinity (predicted ddG affinity of <0 kcal/mol) or introduce a new interaction in the repacked complex structure, while maintain stability (predicted ddG stability <1.0 kcal/mol). The list of the sites and mutations selected for further analysis is shown in Table 21.

Table 21 is the list of positions and mutations introduced to anti-TSLP domain for rational mutagenesis and rational-based phage display library.

TABLE 21
Positions and mutation introduced to anti-TSLP affinity improvement

| | | | Mutations for | Mutations for | Improvement |
|---|---|---|---|---|---|
| | Kabat | | rational | rational phage | as single site |
| CDR | numbering | WT | mutagenesis | display library | mutation |
| CDRH1 | H28 | T | WYFLISH | WYFLISH | No |
| | | | KR | KR | |
| | H30 | R | KHY | KHY | No |
| | H31 | T | HQNVS | HQNVS | No |
| | H32 | Y | FW | FW | No |
| CDRH2 | H52A | Y | QWH | QWH | No |
| CDRH3 | H96 | P | LNQFYDE | LNQFYDE | No |
| | | | HRK | HRK | |
| | H99 | E | QY | QY | Yes, E99Y |
| | H100 | L | DES | DES | No |
| | H100A | V | DES | DES | No |
| | H100B | H | FMWY | FMWY | No |
| | H100D | A | HRKFY | HRKFY | No |
| | H101 | D | W | WSQ | No |
| | H102 | — | YWVL | YWVL | No |
| CDRL1 | L30 | S | DE | DE | No |
| | L32 | S | DE | DE | No |
| CDRL2 | L50 | D | YFRQEKW | YFRQEK | No |
| | | | ST | WSTAGH | |
| | | | | ILPV | |
| | L53 | D | QNSTRKE | QNSTRK | No |
| | | | | EAFIGHP | |
| | | | | QYW | |
| | L55 | P | WYHLIKR | WYHLIKR | No |
| | | | NQ | NQADEF | |
| | | | | GMSTV | |
| | L56 | S | WRLIHVK | WRLIHVK | Yes, 56R or |
| | | | Y | YAEFGNP | 56W |
| | | | | QRT | |
| CDRL3 | L93 | S | DEKR | DE | No |

The mutations for rational design mutagenesis were made as single mutation were cloned into mammalian expression vectors and generated as IgG molecules using standard expression and purification techniques well known in the art.

Rational designed libraries, targeting heavy and light chain CDRs were made as single and combinational mutations. Three VH based soft randomization libraries (4, 5), targeting CDRH1, CDRH2 or CDRH3, were constructed as well coupled with wild type light chain. Mutant phage libraries were rescued, and a two-round solution phase selection approach termed Hammer-hug were performed (4, 5). In brief, phage selections were carried out starting with Hammer selection for round 1 (2 pM biotinylated hTSLP-avi-v5-his10), followed by aggressive off-rate competition overnight (with 2 mM non-biotinylated TSLP-avi-v5-his10) and Hug selection for round 2 (1 pM biotinylated long form human TSLP-avi-v5-his10 in the absence of off-rate competition) split across two branches with or without thermal challenge (70° C.). All outputs were expressed as scFv in crude periplasmic extracts and screened in a competition HTRF assay that assessed performance against the TSLP-0100 scFv (4).

Variants derived from both rational design and soft randomization phage display libraries were cloned into mammalian expression vectors and generated as IgG molecules using standard expression and purification techniques well known in the art.

### Example 21

### Engineering Anti-TSLP Binding Domain to Reduce Viscosity

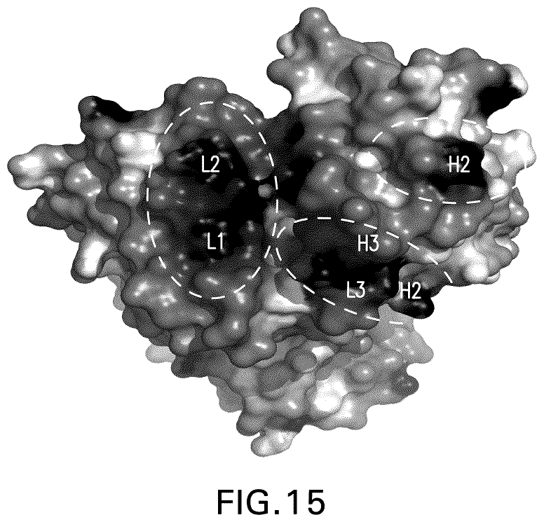

The affinity engineering of TSLP-0001 to TSLP-0260 introduced CDR mutation in the HC E99Y and LC S93E along with germline HC mutations T70S and N79Y. This affinity optimized clone though showed increased viscosity over the parental TSLP-0001 so we sought to identify mutations to reduce the viscosity. It has been seen previously that excess negative charge in the CDR could be a driver of antibody viscosity but attempts to reduce viscosity significantly and maintain affinity have not always been successful (16, 17, 19, 20, 21, 22, 23). In the attempt to reduce viscosity, we identified net positive charge mutations that were tolerated to break up negative charge patches on the IL4IL13TSLP-0260. These charge patches were identified by calculating the electrostatic surface potential using the Poisson Boltzmann Calculator Delphi which is part of Discovery Studio 4.5. In FIG. 15, in black, the negative charge patches are labeled and cover parts of the CDRs L1-L2, L3-H2-H3 and H2 respectively. Using Discovery Studio and FoldX as described for the affinity optimization, we predicted mutations in or surrounding the charge patches that had a net positive charge change but were tolerated in the structure (ddG Affinity <1 kcal/mol and ddg Stability <1 kcal/mol). From this set, we found sites with neutral or negative residues that could tolerate being mutated to Arg or Lys, and negative charged sites that could tolerate to be mutated to neutral or positive residues. The mutations selected for testing are shown in Table 22. In addition, designs were made for the HC combining the E95S mutation with other HC mutants and combining a set of higher confidence LC mutations together in the same cluster or across the two clusters including D52S/S94K, S52K/S94K, D52H/S94K, D50T/S94K, S30K/S94K, S30K/D53S, S30K/S52K, S30K/D51H, S30K/D50T, S52K/D53S, D51H/D53Q, D51H/S52K, D50K/D53Q, D50T/S52K and D50K/D51L.

TABLE 22
List of charge change mutation selected to
potentially reduce viscosity.

| | WT (VH | | | | | |
|---|---|---|---|---|---|---|
| | SEQ ID | | | | | |
| | 16, VL | | | | | |
| Pfabat | SEQ ID | | | | | |
| Site | 18) | Mutants | | | | |
| L29 | G | K | R | | | |
| L30 | S | K | R | | | |
| L32 | S | K | R | | | |
| L50 | D | K | R | S | T | |
| L51 | D | H | K | L | R | |
| L52 | S | K | R | | | |
| L53 | D | K | Q | R | S | |
| L93 | E | K | R | S | | |
| L94 | S | K | R | | | |
| L95 | S | K | R | | | |
| L95a | D | H | K | Q | R | |
| H53 | D | G | K | P | R | T |
| H61 | D | K | Q | R | T | |
| H100 | L | K | R | | | |
| H100a | V | K | R | | | |
| H100c | E | Q | | | | |
| H100d | A | K | R | | | |

Variants derived from these designs were cloned into mammalian expression vectors and generated as IgG molecules using standard expression and purification techniques well known in the art.

### Example 22

### Variants Show Improved Anti-TSLP Bioactivity in Human Primary Monocytes

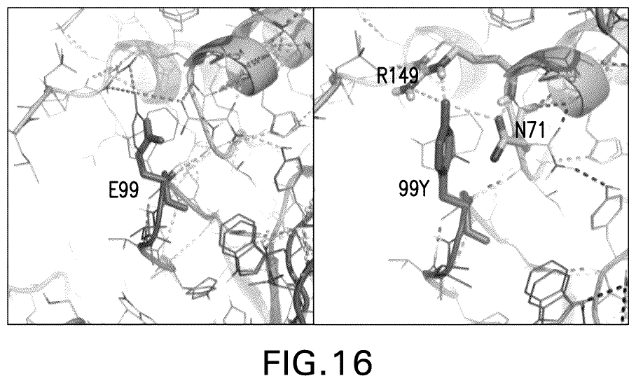

Anti-TSLP neutralizing activity was measured through TARC bioassay in human primary PBMCs. Antibody variants shown improved anti-TSLP bioactivity are listed at Table 23. The activity improvement is illustrated as fold change to parental antibody TSLP-0001. Strikingly all the variants harbor an E99Y (Pfabat numbering) mutation at VH-CDR3 except TSLP-0520 and 0560 show improved bioactivity. TSLP-0821 variant bearing same frameworks and variable regions as parental TSLP-0001 except E99Y mutation, shows 3-fold improved anti-TSLP bioactivity compared with parental TSLP-0001. TSLP-0156 variant (SEQ ID NO: 97 (HC) and 98 (LC)) bearing the same frameworks and variable regions as FW optimized TSLP-0100 except E99Y mutation exhibits 3.9-fold improved anti-TSLP bioactivity compared with parental TSLP-0001. This E99Y mutation was screened out from both rational mutagenesis method and phage display library. Even more, two to seven-fold improved anti-TSLP activity also observed when this E99Y mutation engineered into TSLP-0104 heavy chain (SEQ ID NO: 222 (HC), which has IGVH3-21*02 FW, and paired with various light chains (Table 23, TSLP-0820, 0825, 2000, 2002, 2004). The SEQ ID NOs are in Table 83. Molecular modeling based on co-crystal structure illustrates that this tyrosine at position H99 can potentially form a new hydrogen bond with Asn (N)71 and Arg (R) 149 on TSLP. This will also increase overall packing (FIG. 16). This 2-3-fold of anti-TSLP bioactivity improvement is required in order to maintain a level of TSLP target coverage predicted to be efficacious dosing.

### Example 23

### Anti-TSLP Viscosity Variant Screening

Viscosity for some of the variants with improved anti-TSLP bioactivity was initially screened through single point DLS (dynamic light scattering) bead-based method at highest possible antibody concentration (18, Table 23). Purified antibodies in PBS were extensively dialyzed against 20 mM histidine, 85 mg/mL sucrose, 0.05 mg/mL EDTA pH 6.0 using membrane cassette devices 10K MWCO (Thermo Scientific). Antibodies were concentrated using Viva spin centrifugal concentrators 10K MWCO (GE Healthcare). Sample aliquots (12 μL) were removed from the concentrator retentate as the protein volume was reduced and the protein concentration increased. 300 nm beads (Nanosphere, Thermo Scientific) were added to the protein samples and buffer blank. The beads were diluted 1:10 in 20 mM histidine, 85 mg/mL sucrose, 0.05 mg/mL EDTA pH 6.0 and 0.75 μL diluted beads were spiked into the protein sample. The protein/bead and buffer/bead samples were mixed by gently vortexing. 8 μL sample was transferred to 1536 well plate (SensoPlate, glass bottom, Greiner Bio-One) for analysis by dynamic light scattering measurements (DLS). The plate was sealed with optically clear tape and centrifuged at 2000 RPM for 2 minutes to remove bubbles.

The DLS measurements were made using a DynaPro Plate Reader (Wyatt Technology, Santa Barbara, Calif.). Samples were incubated at 25° C. and measured with 15 consecutive 25 second acquisitions. Radius of the bead was averaged for data acquisitions that had acceptable decay curves. The viscosity was calculated based on the Stokes-Einstein equation. Sample viscosity was calculated as the measured apparent radius divided by the nominal bead radius times 0.893 cP, the viscosity of water at 25° C.

Comparison of viscosity results among parental TSLP-0001, TSLP-0156, TSLP-0260 and TSLP-0708 bearing IGVH3-33 FW demonstrates that reduction of negative charge patch on the surface improves antibody viscosity. 9 variants designed on the light chain mutation to disrupt the negative charge patch show improved viscosity yet maintained bioactivity and were selected for further dose-response viscosity measurement.

TSLP-2000, TSLP-2002 and TSLP-2004 bearing anti-TSLP light chain with D95aK or S52K/S94K or S93K mutation respectively also demonstrated improved viscosity and bioactivity when paired with TSLP-0820 heavy chain which has IGVH3-21*02 FW.

TABLE 23
Variants with improved anti-TSLP bioactivity and viscosity
measurement (Pfabat numbering).

| | | | | a-TSLP TARC | | |
|---|---|---|---|---|---|---|
| | | | | bioassay | | |
| | | | | (fold | | Centipoise |
| | | | | change | Highest | (cP) at |
| Antibody | | | VL | toTSLP- | Concentration | highest |
| Name | Sample ID | VH mutation | mutation | 0001) | (mg/mL) | concentration |
| TSLP- | TSLP-0001 | VH-0001 | VL-0001 | 1.0 | 152.5 | 43.2 |
| 0001 | | | | | | |
| TSLP- | IL413TSLP- | T70S, N79Y | VL-0001 | 0.9 | ND | ND |
| 0100 | 0100 | | | | | |
| TSLP- | IL413TSLP- | T70S, N79Y, | D51H | 2.1 | ND | ND |
| 0874 | 0874 | E99Y | S94K | | | |
| TSLP- | IL413TSLP- | T70S, N79Y, | S93E | 2.1 | ND | ND |
| 0520 | 0520 | L100E, 1102L | | | | |
| TSLP- | IL413TSLP- | T70S, N79Y, | S93E | 2.1 | ND | ND |
| 0698 | 0698 | F29Y, Y52aH, | | | | |
| | | K57E, A60G, | | | | |
| | | D61E, V63A, | | | | |
| | | K64E, P96E, | | | | |
| | | E99Y, | | | | |
| | | A 100dS, 1102L | | | | |
| TSLP- | IL413TSLP- | T70S, N79Y, | S93E | 2.2 | ND | ND |
| 0560 | 0560 | D61E K64E | | | | |
| TSLP- | IL413TSLP- | T70S, N79Y, | D53K | 2.3 | ND | ND |
| 0846 | 0846 | E99Y | | | | |
| TSLP- | IL413TSLP- | T70S, N79Y, | S52K | 2.3 | ND | ND |
| 0862 | 0862 | E99Y | D53S | | | |
| TSLP- | IL413TSLP- | Q1E, V11L, | S93E | 2.3 | 145.3 | 167.7 |
| 0825 | 0825 | R16G, T70S, | | | | |
| | | S74A, T77S, | | | | |
| | | N79Y | | | | |
| TSLP- | IL413TSLP- | Q1E, V11L, | VL-0001 | 2.5 | 148.3 | 105.6 |
| 0820 | 0820 | R16G, T70S, | | | | |
| | | S74A, T77S, | | | | |
| | | N79Y | | | | |
| TSLP- | IL413TSLP- | T70S, N79Y, | VL-0001 | 2.7 | ND | ND |
| 0929 | 0929 | E99Y, L100K | | | | |
| TSLP- | IL413TSLP- | T70S, N79Y, | S30K | 2.8 | ND | ND |
| 0829 | 0829 | E99Y | | | | |
| TSLP- | IL413TSLP- | T70S, N79Y, | S30K | 2.8 | ND | ND |
| 0867 | 0867 | E99Y | S93K | | | |
| TSLP- | IL413TSLP- | T70S, N79Y, | S93E | 3.0 | ND | ND |
| 0707 | 0707 | A59G D61E | | | | |
| | | E99Y | | | | |
| TSLP- | IL413TSLP- | E99Y | VL-0001 | 3.0 | 148.2 | 91.1 |
| 0821 | 0821 | | | | | |
| TSLP- | IL413TSLP- | E99Y | S93E | 3.0 | ND | ND |
| 0824 | 0824 | | | | | |
| TSLP- | IL413TSLP- | T70S, N79Y, | S94K | 3.1 | ND | ND |
| 0849 | 0849 | E99Y | | | | |
| TSLP- | IL413TSLP- | T70S, N79Y, | S93E | 3.4 | 144.6 | 153.0 |
| 0260 | 0260 | E99Y | | | | |
| TSLP- | IL413TSLP- | T70S, N79Y, | G29K | 3.7 | ND | ND |
| 0827 | 0827 | E99Y | | | | |
| TSLP- | IL413TSLP- | T70S, N79Y, | VL-0001 | 3.9 | 159.0 | 107.1 |
| 0156 | 0156 | E99Y | | | | |
| TSLP- | IL413TSLP- | T70S, N79Y, | S93E | 3.9 | 146.2 | 203.5 |
| 0708 | 0708 | D61E K64E | | | | |
| | | E99Y | | | | |
| TSLP- | IL413TSLP- | T70S, N79Y, | S30K | 4.0 | ND | ND |
| 0913 | 0913 | E99Y | S52K | | | |
| | | | S93E | | | |
| TSLP- | IL413TSLP- | T70S, N79Y, | VL-0001 | 4.0 | ND | ND |
| 0928 | 0928 | E99Y, D61K | | | | |
| TSLP- | IL413TSLP- | T70S, N79Y, | S93K | 4.2 | ND | ND |
| 0847 | 0847 | E99Y | | | | |
| TSLP- | IL413TSLP- | T70S, N79Y, | VL-0001 | 4.2 | ND | ND |
| 0930 | 0930 | E99Y, L100R | | | | |
| TSLP- | IL413TSLP- | T70S, N79Y, | S56R | 4.3 | ND | ND |
| 0258 | 0258 | E99Y | | | | |
| TSLP- | IL413TSLP | T70S, N79Y, | S56R, | 4.3 | ND | ND |
| 0261 | 0261 | E99Y | S93E | | | |
| TSLP- | IL413TSLP- | T70S, N79Y, | S52K | 4.8 | 127.3 | 22.1 |
| 0841 | 0841 | E99Y | | | (Solubility | |
| | | | | | Limit) | |
| TSLP- | IL413TSLP- | T70S, N79Y, | S30K | 5.0 | 163.1 | 75.5 |
| 0865 | 0865 | E99Y | S52K | | | |
| TSLP- | IL413TSLP- | T70S, N79Y, | D53S | 5.0 | 160.7 | 151.6 |
| 0872 | 0872 | E99Y | S93K | | | |
| TSLP- | IL413TSLP- | T70S, N79Y, | S52K | 5.0 | 162.1 | 64.3 |
| 0875 | 0875 | E99Y | S94K | | | |
| TSLP- | IL413TSLP- | T70S, N79Y, | D53S | 5.0 | ND | ND |
| 0876 | 0876 | E99Y | S94K | | | |
| TSLP- | IL413TSLP- | Q1E, V11L, | S52K | 5.1 | 150.0 | 34.4 |
| 2002 | 2002 | R16G, T70S, | S94K | | | |
| | | S74A, T77S, | | | | |
| | | N79Y, E99Y | | | | |
| TSLP- | IL413TSLP- | T70S, N79Y, | S56W | 5.2 | ND | ND |
| 0259 | 0259 | E99Y | | | | |
| TSLP- | IL413TSLP- | T70S, N79Y, | D95aK | 5.3 | 158.8 | 47.0 |
| 0855 | 0855 | E99Y | | | | |
| TSLP- | IL413TSLP- | T70S, N79Y, | VL-0001 | 5.3 | ND | ND |
| 0927 | 0927 | E99Y,D61R | | | | |
| TSLP- | IL413TSLP- | T70S, N79Y, | S95K | 6.3 | ND | ND |
| 0851 | 0851 | E99Y | | | | |
| TSLP- | IL413TSLP- | T70S, N79Y, | S52K | 6.3 | 161.8 | 60.8 |
| 0871 | 0871 | E99Y | S93K | | | |
| TSLP- | IL413TSLP- | T70S, N79Y, | S52K | 6.3 | ND | ND |
| 0891 | 0891 | E99Y | S93E | | | |
| TSLP- | IL413TSLP- | T70S, N79Y, | S93E | 6.3 | 157.2 | 74.1 |
| 0903 | 0903 | E99Y | D95aK | | | |
| TSLP- | IL413TSLP- | T70S, N79Y, | S93E | 6.3 | 161.6 | 90.4 |
| 0904 | 0904 | E99Y | D95aR | | | |
| TSLP- | IL413TSLP- | T70S, N79Y, | S52K | 6.3 | ND | ND |
| 0918 | 0918 | E99Y | S93E | | | |
| | | | S94K | | | |
| TSLP- | IL413TSLP- | T70S, N79Y, | VL-0001 | 6.3 | ND | ND |
| 0926 | 0926 | E99Y, D61Q | | | | |
| TSLP- | IL413TSLP- | Q1E, V11L, | S93K | 6.4 | 150.0 | 46.3 |
| 2004 | 2004 | R16G, T70S, | | | | |
| | | S74A, T77S, | | | | |
| | | N79Y, E99Y | | | | |
| TSLP- | IL413TSLP- | Q1E, V11L, | D95aK | 7.2 | 150.0 | 36.3 |
| 2000 | 2000 | R16G, T70S, | | | | |
| | | S74A, T77S, | | | | |
| | | N79Y, E99Y | | | | |
| TSLP- | IL413TSLP- | T70S, N79Y, | S93R | 7.3 | ND | ND |
| 0848 | 0848 | E99Y | | | | |
| TSLP- | IL413TSLP- | T70S, N79Y, | VL-0001 | 7.3 | ND | ND |
| 0922 | 0922 | E99Y, D53T | | | | |
| TSLP- | IL413TSLP- | T70S, N79Y, | VL-0001 | 8.3 | 149.7 | 91.3 |
| 0925 | 0925 | E99Y, D61T | | | | |
| TSLP- | IL413TSLP- | T70S, N79Y, | S30K | 11.0 | ND | ND |
| 0868 | 0868 | E99Y | S94K | | | |

Notes:
VH-0001: variable heavy chain from TSLP-0001. VL-0001: variable lambda chain from TSLP-0001.

### Example 24

### Positions and Combinations that have Impact on Anti-TSLP Antibody Activity

A number of positions and combinations were identified to have negative impact on anti-TSLP activity during screening process via off-rate screening, competition ELISA and TARC bioassay. The antibody variants show fast off-rate, high IC₅₀ in competition ELISA and less potent in TARC bioassay in comparing with parental TSLP-0001. The variants are listed at Table 24. Anti-hTSLP bioactivity is presented as fold change to TSLP-0001: bioactivity of variant/bioactivity of TSLP-0001. Here 50% of TSLP-0001 bioactivity (0.5) was set as cutoff. Significant reduction of anti-TSLP neutralization activity was observed at when proline at light chain positive 55 (P55, Pfabat numbering) was substituted to W, Y, H, L, I K, R, N, Q and in combination with W57G/G78V mutations and paired with either TSLP-0001 heavy chain or framework optimized heavy chain. Substitutions of arginine at position 30 (R30) to lysine in single or in combinations show significant bioactivity reduction. Same observation was seen for light chain aspartic acid at position 50 (050). W52T and Y52a mutation on heavy chain show only 29% of TSLP-0001 anti-TSLP bioactivity.

TABLE 24
Variants that have reduced anti-TSLP activity

| | | | | anti-hTSLP |
|---|---|---|---|---|
| | | | | TARC |
| | | | | bioactivity (fold |
| Antibody | | | | change to |
| Name | Sample ID | VH mutations | VL mutations | TSLP-0001) |
| TSLP-0001 | TSLP-0001 | VH-0001 | VL-0001 | 1.00 |
| TSLP-0100 | IL413TSLP- | T70S, N79Y | VL-0001 | 0.90 |
| | 0100 | | | |
| TSLP-0218 | IL413TSLP- | VH-0001 | W57G, G78V, | 0.10 |
| | 0218 | | P55W | |
| TSLP-0219 | IL413TSLP- | VH-0001 | W57G, G78V, | 0.10 |
| | 0219 | | P55Y | |
| TSLP-0220 | IL413TSLP- | VH-0001 | W57G, G78V, | 0.10 |
| | 0220 | | P55H | |
| TSLP-0221 | IL413TSLP- | VH-0001 | W57G, G78V, | 0.06 |
| | 0221 | | P55L | |
| TSLP-0222 | IL413TSLP- | VH-0001 | W57G, G78V, | 0.11 |
| | 0222 | | P551 | |
| TSLP-0223 | IL413TSLP- | VH-0001 | W57G, G78V, | 0.13 |
| | 0223 | | P55K | |
| TSLP-0224 | IL413TSLP- | VH-0001 | W57G, G78V, | 0.10 |
| | 0224 | | P55R | |
| TSLP-0225 | IL413TSLP- | VH-0001 | W57G, G78V, | 0.23 |
| | 0225 | | P55N | |
| TSLP-0226 | IL413TSLP- | VH-0001 | W57G, G78V, | 0.18 |
| | 0226 | | P55Q | |
| TSLP-0210 | IL413TSLP- | T70S, N79Y | W57G, G78V, | 0.35 |
| | 0210 | | P55N | |
| TSLP-0211 | IL413TSLP- | T70S, N79Y | W57G, G78V, | 0.42 |
| | 0211 | | P55Q | |
| TSLP-0214 | IL413TSLP- | T70S, N79Y | S32D | 0.10 |
| | 0214 | | | |
| TSLP-0192 | IL413TSLP- | T70S, N79Y | D53T | 0.50 |
| | 0192 | | | |
| TSLP-0552 | IL413TSLP- | T70S, N79Y, R30K | S93E | 0.31 |
| | 0552 | | | |
| TSLP-0538 | IL413TSLP- | T70S, N79Y, T281, | S93E | 0.42 |
| | 0538 | R30G | | |
| TSLP-0542 | IL413TSLP- | T70S, N79Y, T28S, | S93E | 0.09 |
| | 0542 | R30T | | |
| TSLP-0557 | IL413TSLP- | T70S, N79Y, T28S, | S93E | 0.32 |
| | 0557 | R30G | | |
| TSLP-0536 | IL413TSLP- | T70S, N79Y, T28S, | S93E | 0.20 |
| | 0536 | F29Y, R30K | | |
| TSLP-0565 | IL413TSLP- | T70S, N79Y, | S93E | 0.29 |
| | 0565 | W52T, Y52aF | | |
| TSLP-0859 | IL413TSLP- | T70S, N79Y, E99Y | D50K D53Q | 0.1 |
| | 0859 | | | |
| TSLP-0836 | IL413TSLP- | T70S, N79Y, E99Y | D50K | 0.2 |
| | 0836 | | | |
| TSLP-0835 | IL413TSLP- | T70S, N79Y, E99Y | D50R | 0.3 |
| | 0835 | | | |
| TSLP-0857 | IL413TSLP- | T70S, N79Y, E99Y | D50K D51L | 0.4 |
| | 0857 | | | |

Notes:
VH-0001: TSLP-0001 variable heavy chain. VL-0001: TSLP-0001 variable lambda chain.

### Example 25

### Anti-TSLP Bioactivity Determination of Lead Variants in Human Primary Monocytes

Variations in the variable regions for TSLP-0156, TSLP-0260, TSLP-0855, TSLP-0871 and TSLP-0875 are listed in Table 25. The anti-TSLP bioactivity was measured by TARC bioassay in human primary PBMCs. All variants show very potent single pM (IC₅₀) TSLP neutralizing activity and 4-fold improvement over parental TSLP-0001.

TABLE 25
TSLP Neutralizing Activity of Anti-TSLP Variants (Pfabat numbering)

| | | | | TARC |
|---|---|---|---|---|
| | | | | bioassay |
| Antibody Name | Sample ID | VH mutation | VL mutation | (IC₅₀, pM) |
| TSLP-0001 | TSLP-0001 | VH-0001 | VL-0001 | 19.1 ± 1.42 |
| TSLP-0156 | IL413TSLP- | T70S, N79Y, | VL-0001 | 4.25 ± 0.59 |
| | 0156 | E99Y | | |
| TSLP-0260 | IL413TSLP- | T70S, N79Y, | S93E | 6.65 ± 0.30 |
| | 0260 | E99Y | | |
| TSLP-0855 | IL413TSLP- | T70S, N79Y, | D95aK | 4.12 ± 0.66 |
| | 0855 | E99Y | | |
| TSLP-0871 | IL413TSLP- | T70S, N79Y, | S52K S93K | 5.69 ± 0.89 |
| | 0871 | E99Y | | |
| TSLP-0875 | IL413TSLP- | T70S, N79Y, | S52K S94K | 5.56 ± 0.95 |
| | 0875 | E99Y | | |

### Example 26

### Characterization of Biophysical Properties and Non-Specificity Scores of Selected Anti-TSLP Variants

Biophysical properties of antibody variants TSLP-0156, TSLP-0260, TSLP-0855, TSLP-0871, TSLP-0875, TSLP-2000, TSLP-2002 and TSLP-2004 variants were further characterized through thermal stability, low pH hold stability, non-specificity, charge Heterogeneity Analysis and viscosity. Data is shown at Table 26 and Table 23. All five antibody variants display comparable or better thermostability than parental TSLP-0001 (also in Table 26). These variants show better low pH hold stability and lower level of acidic species heterogeneity than parental antibody TSLP-0001. These variants also display acceptable level of non-specificity except TSLP-0855 shown high level of self-association propensity.

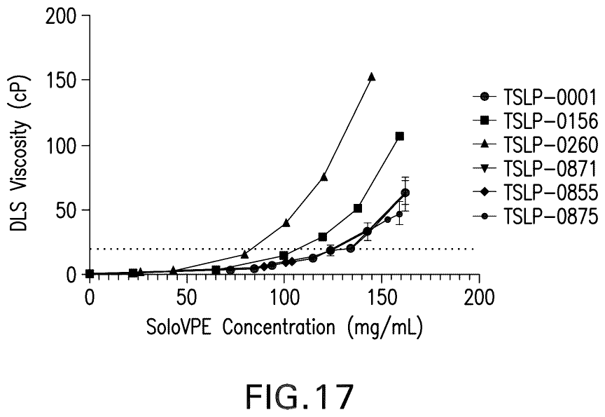

The viscosity was measured through bead-based DLS method with dose response. Removal S93E mutation (TSLP-0156), which disturbed surface negative charge patch, show improved viscosity. Three variants TSLP-0855, TSLP-0871 and TSLP-0875 designed with substitution of positive charged amino acids to reduce the negative charge patch restore the viscosity and displayed comparable viscosity as TSLP-0001 (Table 26 and FIG. 17).

TABLE 26
Characterization of Non-Specificity Scores and Biophysical Properties

| | | | | | | low pH3.4 | | Viscosity |
|---|---|---|---|---|---|---|---|---|
| Antibody | | AC- | DNA | Insulin | | hold | T0 iCE | (mg/mL |
| Name | Sample ID | SINS | score | score | Tm1(° C.) | Δ%HMMS | % Acidic | at 20cP) |
| TSLP-0001 | TSLP-0001 | 0 | 2 | 2 | 66.7 ± 0.10 | 7.3 | 41.2 | 135 |
| TSLP-0156 | IL413TSLP-0156 | 7 | 4 | 3 | 67.46 ± 0.10 | −0.2 | ND | 110 |
| TSLP-0260 | IL413TSLP-0260 | 4 | 5 | 5 | 67.47 ± 0.13 | 0.1 | 28.3 | 90 |
| TSLP-0855 | IL413TSLP-0855 | 17 | 5 | 3 | 70.64 ± 0.00 | 0.17 | 18.5 | 123 |
| TSLP-0871 | IL413TSLP-0871 | 8 | 3 | 3 | 66.40 ± 0.08 | 0.13 | 21.7 | 126 |
| TSLP-0875 | IL413TSLP-0875 | 8 | 3 | 3 | 68.53 ± 0.00 | 0.07 | 16.7 | 122 |
| TSLP-2000 | TSLP-2000 | 7* | 7* | 10# | 70.01 ± 0.00 | −0.10 | 21.8 | |
| TSLP-2002 | TSLP-2002 | 6* | 7* | 10# | 67.74 ± 0.00 | −0.01 | 32.4 | |
| TSLP-2004 | TSLP-2004 | 5*** | 5*** | 7* | 66.58 ± 0.00 | −0.03 | 23.5 | |

Wot reference to only the lowest three rows (TSLP-2000, −2002, −2004), # denotes sub-optimal, *denotes acceptable, and *** denotes advantageous.

### Derivation and Characterization of Anti-p40 Fab Binding Domain

### Example 27

### Derivation of Anti-IL12/p40 Binding Domain p40-0003 Antibody

IL-12p40 (or referred also as p40) is a shared subunit of the heterodimeric cytokines IL-12 which is composed of p40 and p35 subunits and IL-23 which is composed of p40 and p19 subunits. The approach used to generate anti-p40 binding domain was to derive the human anti-p40 antibody C230 (ustekinumab, Stelara®) VH and VL obtained from Giles-Komar et al. U.S. Pat. No. 6,902,734 (Centocor Inc., Jun. 7, 2005). The C230 V_{L} was fused to the human Kappa constant region (SEQ ID NO: 16) within a proprietary expression vector and to generate p40-0003 LC. The C230 V_{H} was fused to the human IgG1 constant region (SEQ ID NOs: 6,7,8,9) within a proprietary expression vector with mutations in CH1 that eradicates effector function (Pfabat numbering: L247A, L248 Å, G250A; EU number: Leu234Ala, Leu235Ala and Gly237Ala, SEQ ID: 6) to generate p40-0003 HC. Expi293F™ HEK cells were transiently transfected with DNA encoding p40-0003 LC and HC and purified by MabSelect™ SuRe™ column to generate anti-p40 antibody (p40-0003).

### Example 28

### Kinetic Evaluation of Anti-p40 p40-0003 Antibody Binding Human and Cyno IL-12 and IL-23 Using Surface Plasmon Resonance

Surface plasmon resonance (SPR) was performed to determine the affinity constants for antibody p40-0003 against human and cynomolgus IL-12 and human and cyno IL-23 that contain p40 subunit. For these analyses, kinetic assays were conducted at 37° C. at a collection rate of 10 Hz on a BIAcore™ T200 instrument (GE Healthcare). Anti- human IgG antibody (anti-human Fc, catalog #109-005-098, Jackson ImmunoResearch) was amine coupled to all four flow cells of a carboxymethylated dextran coated sensor chip (CM5) (GE Healthcare) using the manufacturer's protocol. Next, p40-0003 mAb was immobilized to ˜100 Response Units (RUs) and various concentrations of human IL-12 or cyno IL-12 or human IL-23 or cyno IL-23 were injected. The measured affinity constants of p40-0003 antibody against human and cynomolgus IL-12 and IL-23 are presented in Table 27 below. Anti-p40 p40-0003 antibody binds to human IL-12 and IL-23 with low pM affinity (Table 27). P40-0003 also has ˜2-3 fold greater affinity to human IL-12 and human IL-23 than cyno IL-12 and IL-23.

TABLE 27
Affinity Constants Obtained using Surface Plasmon Resonance

| | p40 mAb | | | |
|---|---|---|---|---|
| | P40-0003 | | | |
| | Human IL-12 | Cyno IL-12 | Human IL-23 | Cyno IL-23 |
| Affinity (pM) | 156 ± 7.07 | 368 ± 12.0 | 159.5 ± 0.71 | 316.5 ± 40.3 |

### Example 29

### Engineering Tri-Fab-Fc Variants Using Different Molecular Formats and Heterodimerization Mutations to Minimize Byproducts

Several multifunctional format designs were engineered to identify molecules that could simultaneously bind and neutralize three different targets while conferring developability parameters of a standard monoclonal antibody. Design considerations for engineering the trifunctional variants were to address multiple challenges: in addition to binding to and blocking 3 targets at once, the molecule needed efficient pairing of five different protein chains, high-level protein expression with few undesired byproducts, high transient and stable expression titers, efficient cell line generation and protein purification processes, and pharmacokinetic properties consistent with a standard monoclonal antibody.

All molecules were built with the same general structure based on human IgG1. The human IgG1 fragment crystallizable region (Fc region), normally a homodimer, was engineered to form Fc heterodimers preferentially by asymmetrically placed mutations at the Fc interface (see below). One of the asymmetric Fc regions was linked to a single Fab domain via a human IgG1 hinge and is referred to as the single Fab arm (SFab). The other asymmetric Fc is linked via a human IgG1 hinge to an inner Fab domain, which in turn is linked to an outer Fab domain; this is referred to as the dual Fab arm (DFab; FIG. 18). DFab arm designs vary, but the outer Fab (referred to as the Fab1 position) is always linked to the inner Fab (Fab2 position) by a single short flexible linker (SEQ ID NO: 104). The Fab in the single Fab arm is referred to as the Fab3 position.

FIG. 18 indicates the positions of Fab1, Fab2, and Fab3, and illustrates the positions of the dual Fab (DFab) and single Fab (SFab) arms. Multiple arrangements of variable heavy (V_{H}) and variable light (V_{L}) regions, as well as of the first constant domain of the heavy chain (CH1) and the light chain constant domains (Cκ or Cλ), are also illustrated in FIG. 18 and will be described in detail in the following examples.

For clarity, the term “chain” will be used to refer to single polypeptide chains, while the term “arm” will be used to refer to paired polypeptide chains (e.g., the SFab arm consists of a paired heavy chain and light chain). The intact trispecific molecule will be referred to as a Tri-Fab-Fc. Because the Tri-Fab-Fc molecules contain two or more light chains, the name of each chain also includes a number to indicate the applicable Fab position. The SFab arm, which contains the Fab in the Fab3 position, consists of a heavy chain, referred to as the SFab HC(3), and a light chain, referred to as the SFab LC(3). The DFab arm consists of a dual Fab chain paired with two other chains, depending on the format. For example, a DFab arm using the modified Fd format (FIG. 18) is composed of a dual Fab chain referred to as DFab LC(1)-HC(2), a light chain referred to as DFab LC(2), and a chain referred to as modified Fd (1), which contains the VH of Fab1 linked to a CH1 domain. In this example, DFab LC(2) pairs with the VH and CH1 in the Fab2 position, and modified Fd (1) pairs with the VL and Ck in the Fab1 position.

Two approaches were used for heterodimerization of the IgG Fc region. The first approach was done by pairing of two different heavy chains driven by engineering a protuberance (‘bump’ or ‘knob’) at the interface of one heavy chain CH3 domain and a corresponding cavity (hole) in the interface of the second heavy chain CH3 domain, such that the protuberance can be positioned in the cavity as to promote heterodimer formation and hinder homodimer formation (9, 10). This latter approach will be referred to herein as Knob-into-Hole (KiH). Specifically, the Knob mutation T(389)W with Y(370)C (Pfabat numbering) was engineered into one of the IgG1-CH3 domains and the Hole mutations T(389)S, L(391)A and Y(438)V with S(375)C (Pfabat numbering) were introduced into the second IgG1-CH3 domain. [Molecules using this heterodimerization strategy will be described in Examples 30, 31, 32, 33, 34, 35]. The second approach used was where two antibodies or antibody-based molecules are expressed separately in dual cell lines or dual transiently expressed pools, one engineered with excess positive charge and the other with excess negative charge in the complementary location at the dimer interface (12, WO2011/143545). The two antibodies are purified separately, mixed, and then reduced under appropriate conditions to allow for oxidation that will result in preferential formation of a heterodimer bispecific or trispecific molecule. This latter approach will be referred to here on as charge-based (CB) mutations for dual cell production. The Knob-into-Hole Fc heterodimerization mutations were also evaluated for the dual cell approach. One advantage for producing bispecific molecules using a post-expression chemical redox approach is that each heavy chain-light chain pair is expressed in a separate cell line, eliminating mispairing of cognate light chains. However, adding a third Fab binding domain for a trispecific molecule requires co-expression of two heavy-light chain pairs in one cell, leading to complications that need to be resolved using other engineering designs. (Molecules using dual-cell approaches will be described in Example 41).

To facilitate efficient production of trifunctional molecules using five unique protein chains, multiple design strategies were employed to incorporate the third Fab binding domain and mitigate promiscuity of light chain mispairing, which leads to formation of numerous undesired biproducts. (These designs are described in Examples 31-35).

### Example 30

### Single Cell Produced Trispecifics with Unmodified Fab1 Format

To minimize light chain mispairing when all five protein chains were expressed in in a single cell, the electrostatic complementary S1 and S1-reverse mutations (11) were introduced into the Fab2 and Fab3 human Kappa (CL) and human IgG1-CH1 constant domains (FIG. 18). For the S1 design, a negative charge was engineered into the human Kappa CL by replacing S(L176)D along with the supporting mutation V(L133)S (SEQ ID NO: 113). The complementary S1 design positive charge L(H124)K was introduced into the Fab3 CH1 with the supporting mutation V(H190)S (SEQ ID NO: 110). For the S1-reverse design, a positive charge was engineered onto the human Kappa CL by replacing S(L176)K along with the supporting mutation V(L133)S (SEQ ID NO: 108). The complementary S1-reverse design negative charge L(H124)E was introduced into the CH1 with the supporting mutation S(H188)G (SEQ ID NO. 105). The constant regions of the domain in the Fab1 position were not modified.

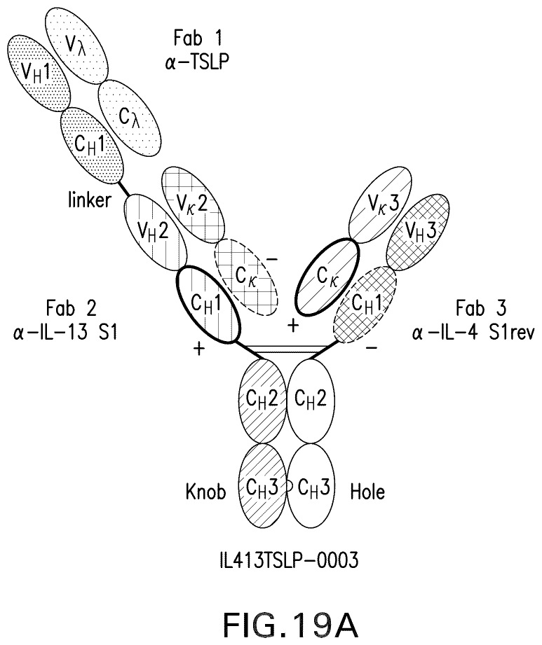

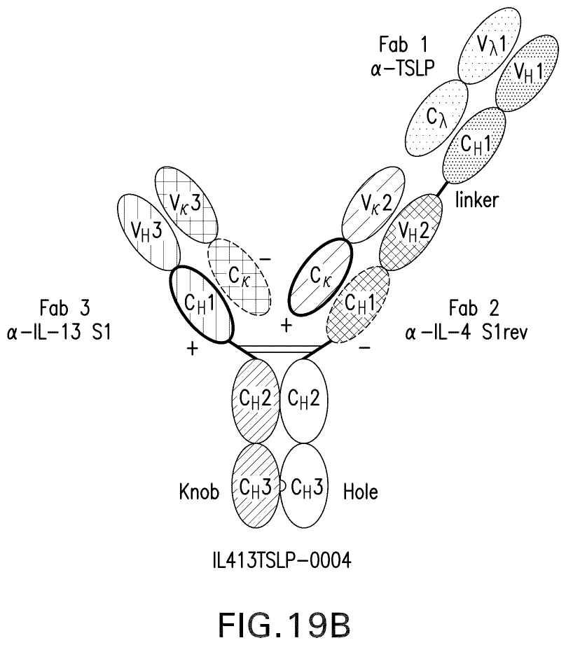

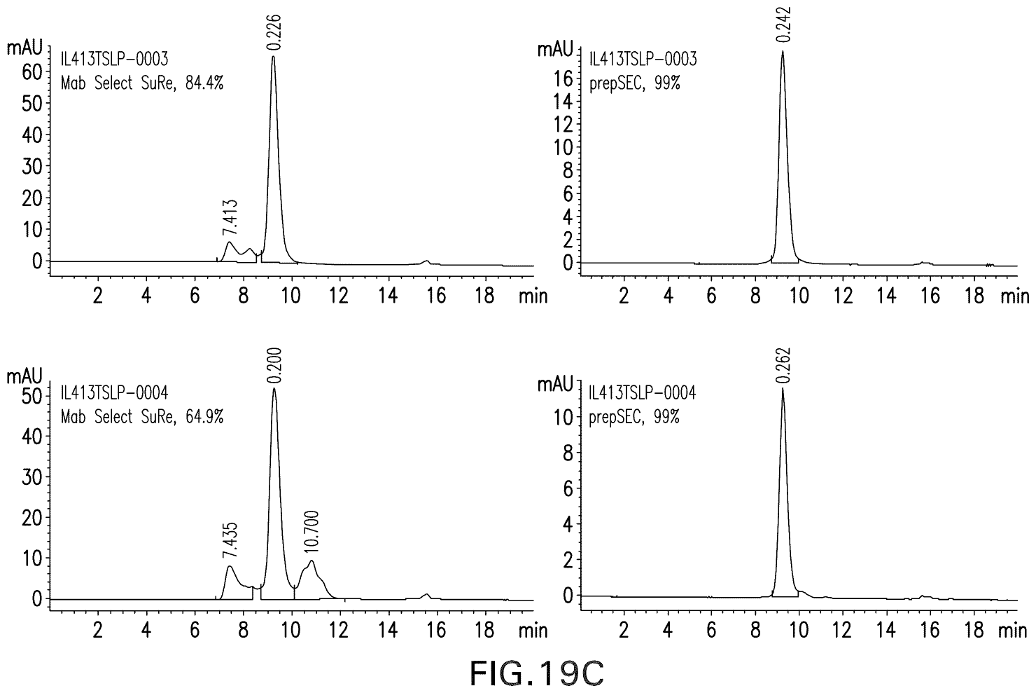

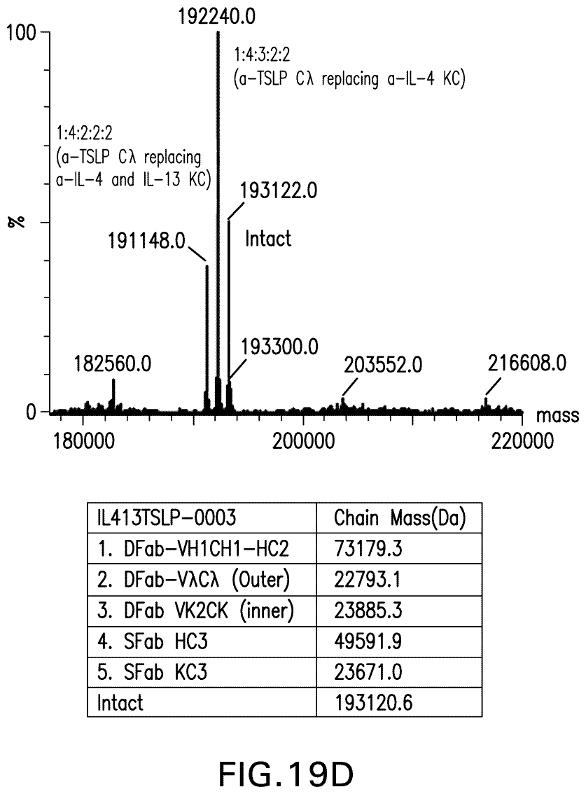

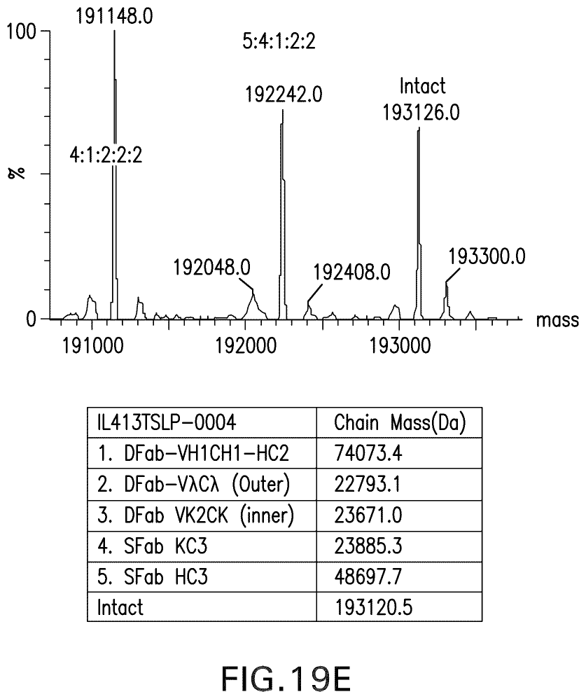

Two trispecific molecules (IL413TSLP-0003 (SEQ ID NOs: 109, 196, 146, 98, and 152) and IL413TSLP-0004 (SEQ ID NOs: 109, 112, 196, 98 and 153)) containing Fabs against TSLP, IL-4, and IL-13 were constructed (FIG. 19a 19b), in which the S1 and S1-reverse mutations were used with IL-4 and IL-13 domains in the Fab2 and Fab3 positions, while the sequences of the TSLP Fab in the Fab1 position did not contain modifications to influence chain pairing. An anti-TSLP Fab domain was designed as conventional Fab placed in the Fab1 position (outer domain of dual Fab arm), with either an anti-IL-13 domain (in Tri-Fab-Fc IL413TSLP-0003) or an anti-IL-4 domain (in Tri-Fab-Fc IL413TSLP-0004) in the Fab2 position (inner domain of the dual Fab arm). The anti-TSLP Fab was linked to anti-IL-13 or anti-IL-4 domain through the linker GGGGS (SEQ ID NO: 104). Trispecifics in this format were expressed by co-transfection of five plasmids, which encoded each of the protein chains, in Expi293F™ cells.

High quality DNAs intended for mammalian transfection were prepared through Qiagen endo-free Maxi/Giga kit (Qiagen). Five expression vectors encoding the Tri-Fab-Fc variants were co-transfected into 200 mL Expi293F™ cells according to manufacture protocols (Thermo Fisher, Cat #A14635). The conditioned medium was harvested on day 5 and captured by 5 mL HiTrap MabSelect™ SuRe™ LX (GE Healthcare Life Sciences) followed by a preparative SEC Superdex 200 column (GE Healthcare Life Sciences). Samples that required HPLC analytical SEC (aSEC) analysis were run in a YMC-Pack Diol-200 SEC column using a buffer containing 20 mM sodium phosphate and 400 mM NaCl at pH 7.2. Injection volumes of 5 μL molecular weight standards, 25 μL AAB001, and 50 μg per sample were used, drawing and ejecting both at 150 μL/minute. The retention time and peak width of the main peak as well as the areas and percent areas of the main, low-molecular mass species (LMMS), and high-molecular mass species (HMMS) peaks were recorded.

Transient expression yields of IL413TSLP-0003 and IL413TSLP-0004 Tri-Fab-Fc molecules following Protein A capture were 12.34 and 14.33 mg/L, respectively. The two differed in the proportion of molecules with the correct apparent molecular weight (MW) in material captured on Protein A. IL413TSLP-0003 had 84.4% of the expected theoretical-sized peak on analytical size exclusion chromatography, FIG. 19c, while IL413TSLP-0004 had 64.9%), but both could be further purified to >97% purity using size exclusion chromatography.

Liquid chromatography-mass spectrometry (LC/MS) analysis was carried out to evaluate chain pairing of the Tri-Fab-Fc. The molecular weight of the Tri-Fab-Fc molecule is defined by the unique amino acid sequences of each chain, and accurate molecular weight determination provides evidence for the presence of correctly paired and mispaired molecules. For intact molecular analysis, the Tri-Fab-Fc sample was incubated with recombinant PNGaseF (New England Bio Labs) for 1 hour at 37° C. to remove N-linked oligosaccharides. For reduced chain analysis, the deglycosylated Tri-Fab-Fc was reduced by guanidine and DTT. Next, 25ug of sample was injected onto a BioResolve Polyphenyl 450 Å column and analyzed by LC/MS analysis on a Waters Acquity H-Class HPLC coupled with a Bruker maXis II Q-ToF mass spectrometer. Intact LC/MS analysis of the preparative SEC purified final peaks (FIG. 19d, 19e) showed that TSLP domain light chain in the Fab1 position ((DFab LC(1)) of both IL413TSLP-0003 and IL413TSLP-0004 could pair not only with the correct TSLP VH-CH1 in the DFab VH (1) CH1-HC(2) chain, but also with the IL-4 and IL-13 VH-CH1 domains, whether present in the DFab HC(1)-HC(2) or SFab HC(3) chains, indicating that additional engineering measures needed to be taken to prevent light chain mispairing.

### Example 31

### Constant-Light Domain Swap (CkS and CλS) and V-Region Swap (VDS) Designs for Single Cell Expression of Five Unique Protein Chains

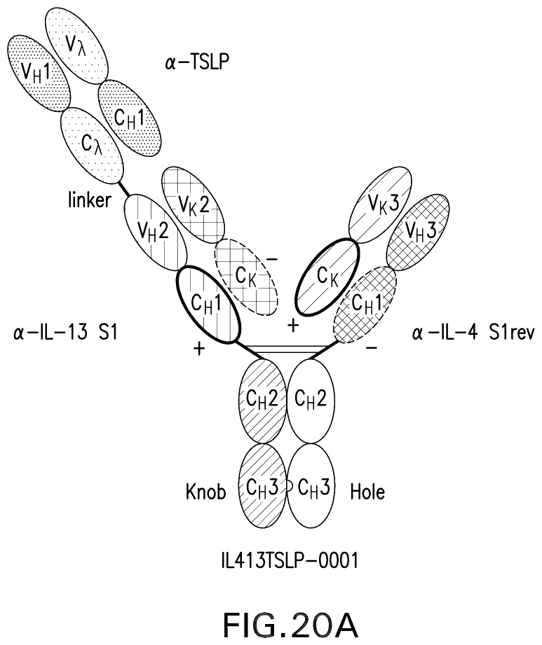

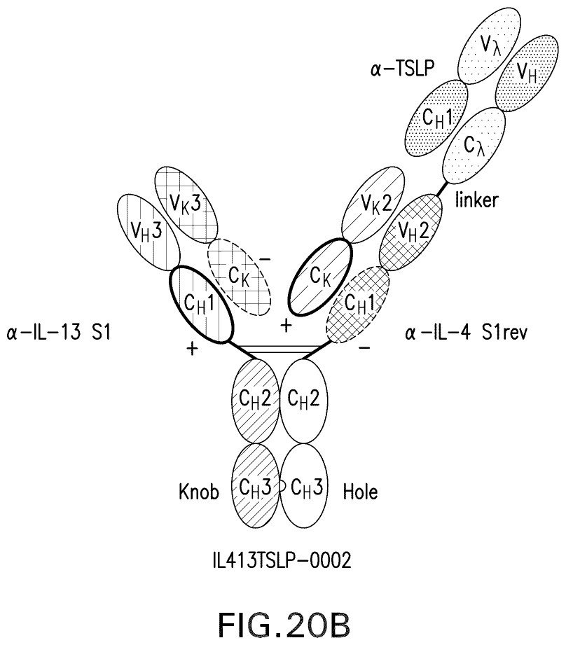

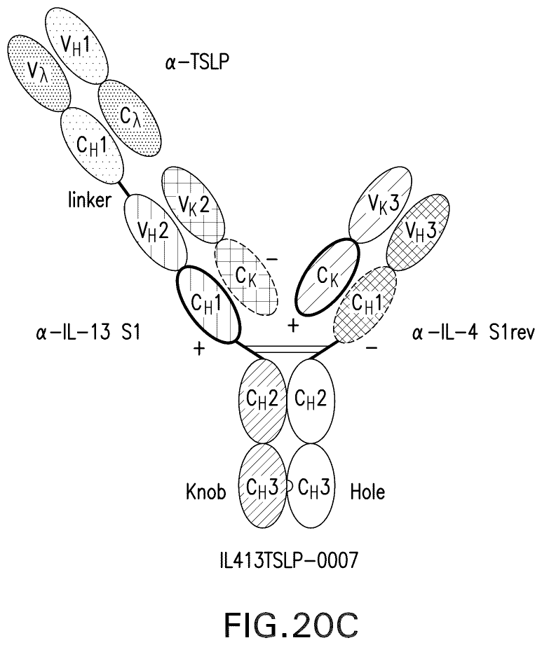

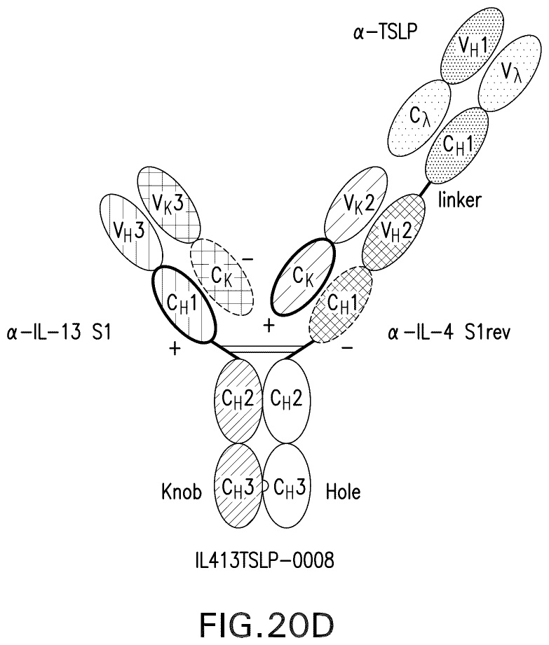

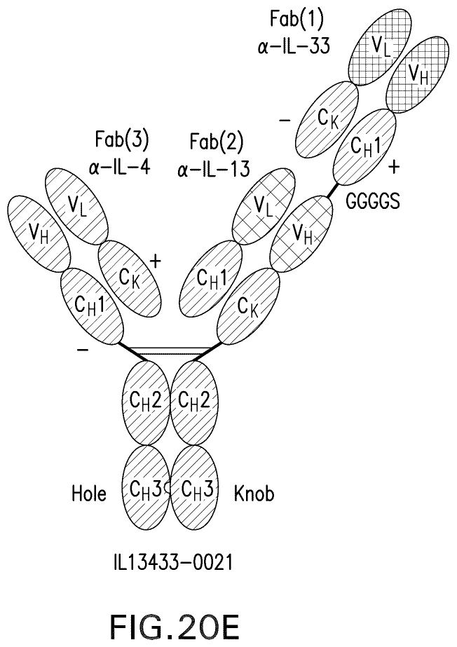

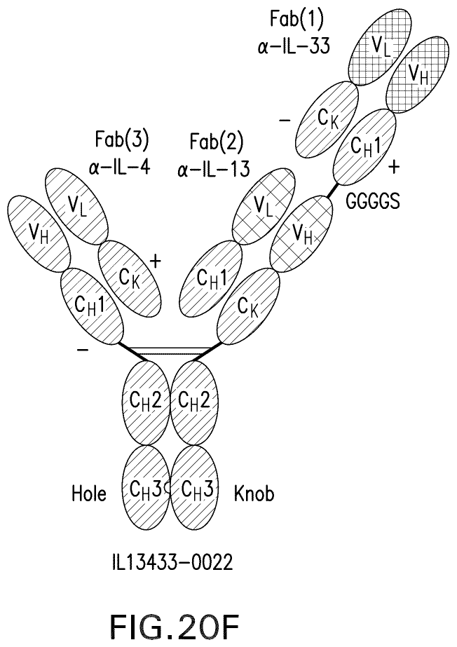

In one set of designs to improve fidelity of Fab assembly, chain pairing of the Fab in the Fab1 (outer) position was driven by modifying domain arrangements within Fab1, either by exchange of variable heavy and light (VH and VL) domains or exchange of constant light domains (C kappa or C lambda) and the heavy chain CH1 domain (FIG. 20A- FIG. 20D). In the V domain swapped (VDS) configuration, a modified light chain consisted of a VH linked to a CL domain instead of the normal VL-CL, and the corresponding portion of the dual Fab chain consisted of a VL linked to CH1 domain instead of the normal VH-CH1. In the C kappa swapped (CkS) configuration, a modified light chain consisted of a VL linked to a CH1 domain, and the corresponding portion of the dual Fab chain consisted of a VH linked to a constant (C) kappa domain. The modified light chain also contained a stretch of amino acids from the IgG1 upper hinge region, EPKSC (SEQ ID NO: 102), added to the C-terminus of the CH1 to form a disulfide bond with the C kappa domain on the dual Fab chain. Similarly, in the C lambda swapped (CλS) configuration, a modified light chain consisted of a VL linked to a CH1 domain, with a C-terminal EPKSC (SEQ ID NO: 102) extension, and the corresponding portion of the dual Fab chain consisted of a VH linked to a CA domain. In each case, the modified domain arrangements were expected to favor the pairing of the corresponding modified chains (e.g., VH-CK with VL-CH1).

Examples of Tri-Fab-Fc antibodies using the designs described above were made with antibodies to IL-4, IL-13, and a third antibody to either IL-33 or TSLP. Correct chain pairing of domains in the Fab2 and Fab3 positions was maintained by use of the S1 and S1rev complementary charge mutations. Specific locations of chain-pairing mutations are described in Table 28.

TABLE 28
Expression of Tri-Fab-Fc Variants Using CλS and VDS in Fab1 Position and
S1/S1rev in Fab2 and Fab3 Positions

| | | | | | | % | | | |
|---|---|---|---|---|---|---|---|---|---|
| Antibody or | | | | | Titer (ProA, | main | | | pH Hold |
| Tri-Fab-Fc | Fc | | | | mg/L) | peak | MS (after | Tm1 | at 3.4 |
| Name | format | Fab1 | Fab2 | Fab3 | Expi293F ™ | (ProA) | prepSEC) | (° C.) | ↑%HMMS |
| TSLP-0001 | IgG | | | | | | | 66.7 | 7.3 |
| TSLP-0100 | IgG | | | | | | | 69.1 | 0.2 |
| IL413TSLP- | KiH | TSLP- | IL13- | IL4- | 2.7 | 78.8 | Intact | 56.99 ± 0.05 | |
| 0001 | | 0001 | 0001 | 0002 | | | | | |
| | | CλS | S1 | S1rev | | | | | |
| IL413TSLP- | KiH | TSLP- | IL4- | IL13- | 3.4 | 54.6 | Intact | 56.84 ± 0.05 | 51.2 |
| 0002 | | 0001 | 0002 | 0001 | | | | | |
| | | CλS | S1rev | S1 | | | | | |
| IL413TSLP- | KiH | TSLP- | IL13- | IL4- | ND | ND | ND | 59.90 ± 0.02 | 15.9 |
| 0249 | | 0100 | 0001 | 0002 | | | | | |
| | | CλS | S1 | S1rev | | | | | |
| IL413TSLP- | KiH | TSLP- | IL13- | IL4- | 7.7 | 74.1 | 95% | 56.97 ± 0.06 | |
| 0007 | | 0001 | 0001 | 0002 | | | Intact | | |
| | | VDS | S1 | S1rev | | | 5% | | |
| | | | | | | | mispairing | | |
| IL413TSLP- | KiH | TSLP- | IL4- | IL13- | 7.2 | 41.0 | Intact | 56.90 ± 0.05 | |
| 0008 | | 0001 | 0002 | 0001 | | | | | |
| | | VDS | S1rev | S1 | | | | | |

ND: not done

TABLE 29
Activity of Tri-Fab-Fc Variants Using CλS and VDS in Fab1 Position and
S1/S1rev in Fab2 and Fab3 Positions

| | | | | | a-TSLP TARC | a-hIL-4 | a-hIL-13 |
|---|---|---|---|---|---|---|---|
| Antibody or | | | | | bioassay | bioassay | bioassay |
| Tri-Fab-Fc | | | | | (IC₅₀; | (IC₅₀; | (IC₅₀; |
| Name | Fc format | Fab1 | Fab2 | Fab3 | pM) | pM) | pM) |
| IL-4-0002 | IgG | | | | | 3.42 | |
| IL-13-0001 | IgG | | | | | | 4.83 |
| TSLP-0001 | IgG | | | | 2.32 | | |
| IL413TSLP- | KiH | TSLP-0001 | IL13- | IL4-0002 | 12.6 | 7.15 | 47 |
| 0001 | | CλS | 0001 | S1Rev | | | |
| | | | S1 | | | | |
| IL413TSLP- | KiH | TSLP-0001 | IL4- | IL13- | 16.4 | 8.59 | 10.6 |
| 0002 | | CλS | 0002 | 0001 | | | |
| | | | S1Rev | S1 | | | |
| IL413TSLP- | KiH | TSLP-0100 | IL13- | IL4-0002 | ND | ND | ND |
| 0249 | | CλS | 0001 | S1Rev | | | |
| | | | S1 | | | | |
| IL413TSLP- | KiH | TSLP-0001 | IL13- | IL4-0002 | 11.7 | 7.09 | 46.8 |
| 0007 | | VDS | 0001 | S1Rev | | | |
| | | | S1 | | | | |
| IL413TSLP- | KiH | TSLP-0001 | IL4- | IL13- | 17.7 | 9.07 | 10.6 |
| 0008 | | VDS | 0002 | 0001 | | | |
| | | | S1Rev | S1 | | | |

ND: not done

The Tri-Fab-Fc variants were transfected and purified via MabSelect™ SuRe™ LX capture followed by MonoQ anion exchange chromatography. The results shown in Tables 28 and 29 indicate that molecules of the CλS, CκS, and VDS formats could be produced and that each binding domain in these formats is capable of engaging its target. Mass spectrometry analysis (Table 28) showed little or no chain mispairing in the molecules examined, IL413TSLP-0001 (SEQ ID NOS: 109, 196, 146, 149 and 150) and IL413TSLP-0002 ((SEQ ID NOS: 109, 112, 196, 150 and 151); Fab1 in CaS format) and IL413TSLP-0007 (SEQ ID NOS: 109, 196, 146, 154 and 155) and IL413TSLP-0008 ((SEQ ID NOS: 109, 112, 196, 155 and 156); Fab1 in VDS format). This observation indicated that engineering of the Fab1 domain arrangement overcame the mispairing observed in IL413TSLP-0003 and IL413TSLP-0004, which lacked modifications to drive pairing of Fab1.

Neutralization of TSLP by all four molecules with the anti-TSLP domain TLSP-0001 in the Fab1 position (IL413TSLP-0001 and IL413TSLP-0002 (Fab1 in CλS format) and IL413TSLP-0007 and IL413TSLP-0008 (Fab1 in VDS format) was similar (Table 29). This observation indicated that both CaS and VDS Fab1 domain arrangements support similar antibody binding activity in the Fab1 position. Potency of neutralization by the Fab in the Fab2 position was also similar in the CaS and VDS constructs (compare IL-4 neutralization by IL413TSLP-0002, IC₅₀ 8.59 pM, CaS, and IL413TSLP-0008, IC₅₀ 9.07 pM, VDS; also compare IL-13 neutralization by IL413TSLP-0001, IC₅₀ 47 pM, CaS, and IL413TSLP-0007, IC₅₀ 46.8 pM, VDS; Table 29). This observation indicated that the specific CλS or VDS arrangement of the Fab in the Fab1 position has little impact on binding by the Fab in the Fab2 position.

In an IgG format, TSLP-0100 exhibited improved thermostability (69.1° C. Tm1) and improved low-pH hold ability and reduced acidic species at T0 iCE study over IL413TSLP-0001 (66.7° C. Tm1) (Table 19 and 28). The same biophysical property improvements in the IgG are evident in Tri-Fab-Fc (Table 28), indicating the translatability from conventional IgG to Tri-Fab-Fc.

The bioactivities of different binding domains were affected differently by being placed in the Fab2 position (i.e., with another Fab fused to the N-terminus) compared to what was observed when they were in the Fab3 position (without any fused protein at the N-terminus). The IL4-0002 domain showed equivalent neutralization activity when present at Fab2 (IL413TSLP-0002, IC₅₀ 8.59 pM; IL413TSLP-0008, IC₅₀ 9.07 pM) and at Fab3 (IL413TSLP-0001, IC₅₀ 7.15 pM, and IL413TSLP-0007, IC₅₀ 7.09 pM; Table 29), indicating that this format of Tri-Fab-Fc molecule is compatible with full activity of the domain at the Fab2 position. However, the IL13-0001 domain was sensitive to its position in the molecule, showing substantial reduction in activity when in the Fab2 position relative to its activity in Fab3 (compare IL413TSLP-0002, IC₅₀ 10.6 pM at Fab3, with IL413TSLP-0001, IC₅₀ 47 pM at Fab2; and IL413TSLP-0008, IC₅₀ 10.6 pM at Fab3, with IL413TSLP-0007, IC₅₀ 46.8 pM at Fab2). As described in Example 33, the sensitivity of IL13-0001 to placement in the Fab2 vs Fab3 position was itself dependent on the domain present at the Fab1 position, in some cases showing little reduction in IL13-0001 activity when in the Fab2 position. Taken together, these results indicate that the TriFab-Fc format is compatible with full activity of the Fab2 domain, but that both the Fab2 domain itself and the Fab1 domain can influence whether a particular binding domain is fully active when in the Fab2 position.

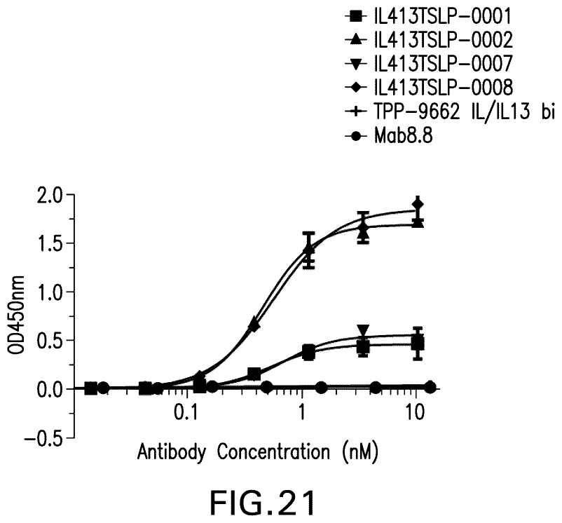

Further underscoring the effect of domain position on activity, a bridging sandwich ELISA (FIG. 21) showed that the placement of IL13-0001 in the Fab2 and Fab3 positions led to significant differences in the ability of two IL413TSLP Tri-Fab-Fc variants to bind the IL-13 and TSLP targets simultaneously. For this method, 25 μL/well of 1 μg/mL recombinant human TSLP (in PBS pH7.2) was coated onto a 384-well clear flat bottom Maxisorp plates (NUNC) overnight at 4° C. and blocked with PBS without Mg²⁺ and Ca²⁺ with 3% BSA. Serially diluted Tri-Fab-Fc variants were added to the plates and incubated for 1 hour at room temperature (RT). After washing away the unbound Tri-Fab-Fc by washing with buffer (PBS without Mg²⁺ and Ca²⁺ with 0.05% tween 20), 25 μL/well of 100 ng/mL IL-13-FLAG (Pfizer Inc) was added to the plates and incubated at RT for one hour. The Tri-Fab-Fc bound to both TSLP and IL-13 was detected by HRP conjugated anti-FLAG secondary antibody (Sigma, Cat #A8592). Significantly lower binding signals were observed for IL413TSLP-0001 and IL413TSLP-0007 (in which the IL13-0001 domain was located in the Fab2 position) than for IL413TSLP-0002 and IL413TSLP-0008 (in which the IL13-0001 domain was located in the Fab3 position). This observation is consistent with the lower IL-13 neutralizing activity of the two Tri-Fab-Fc variants in which IL13-0001 was located in the Fab2 position.

Similarly, to its effects on binding activity, domain position also affected the proportion of molecules of the correct size during expression. Size-exclusion chromatography of material purified on Protein A (Table 28) indicated that the placement of IL4-0002 at Fab3 and IL13-0001 at Fab2 led to a substantially higher fraction of protein at the correct size than the opposite arrangement (compare Tri-Fab-Fcs with IL13-0001 at Fab2 (IL413TSLP-0001, 79-91% correct, and IL413TSLP-0007, 74-85% correct) with Tri-Fab-Fcs with IL13-0001 at Fab3 (IL413TSLP-0002, 52-54% correct, and IL413TSLP-0008, 41-47% correct).

Taken together, the impact of individual binding domains on overall molecular behavior and the impact of molecular structure on the activity of individual binding domains indicate that empirical determination of suitability of specific binding domains for this format is necessary.

### Example 32

### Improvement of IL413TSLP Tri-Fab-Fc Chain Pairing by Adjusting DNA Ratio of Expression Vectors in Transient Transfection

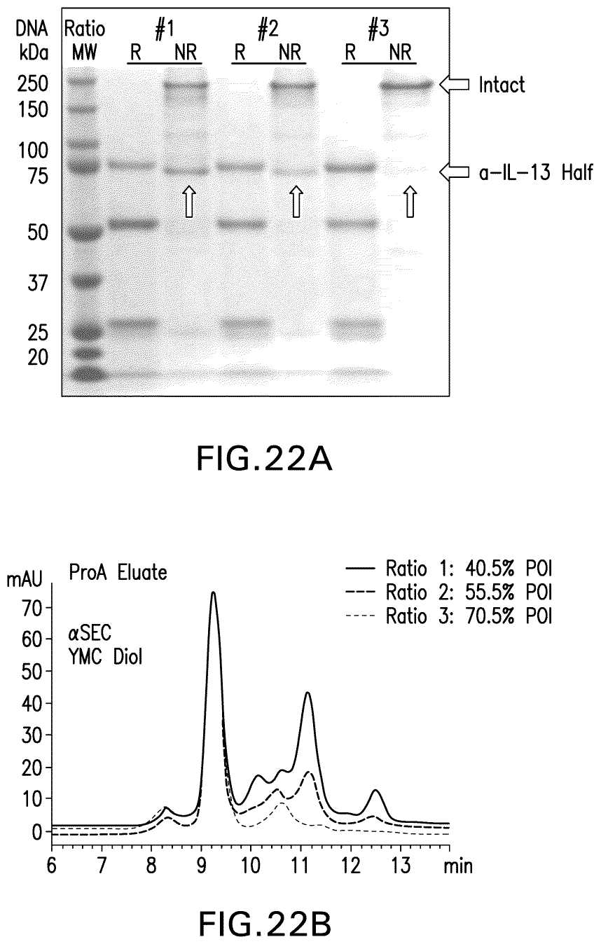

cDNAs encoding each of 5 chains of IL413TSLP-0002 with three different DNA ratios (Table 30) were co-transfected into ExpiCHO-S cells according to the manufacturer's protocol (Thermo Fisher, Cat #A29133). The conditioned medium was harvested and captured by 5 mL HiTrap MabSelect™ SuRe™ LX (GE Healthcare Life Sciences). The Protein A eluates were treated with 4× sample buffer (ThermoFisher, Cat #NP0007) with or without reducing agent dithiothreitol (DTT). The samples were then loaded and analyzed on NuPAGE Bis-tris gel (ThermoFisher, Cat #NP0321 BOX) (FIG. 22a). The NuPAGE data under non-reducing conditions show a significant reduction of a 75 kDa band and an increase of the intact molecule band. LC/MS analysis revealed that the 75 kDa band was unpaired SFab arm anti-IL-13 domain. Analytical SEC data (FIG. 22b; Table 30) show that the percentage of POI (intact molecule) increased from 40% to 70% by adjusting the DNA ratio. Our data demonstrate that manipulating DNA transfection ratios can further drive proper chain pairing.

TABLE 30
DNA ratio for transfection of IL413TSLP-0002

| | Domain | DNA Tx | DNA Tx | DNA Tx |
|---|---|---|---|---|
| IL413TSLP-0002 | description | ratio #1 | ratio #2 | ratio #3 |
| 1: sFab HC (3) | a-IL-13 HC | 1 | 0.75 | 0.5 |
| 2: sFab LC (3) | a-IL-13 kC | 1 | 0.75 | 0.5 |
| 3. DFab VHCL(1)- | a-TSLP-IL- | 1 | 1.25 | 1.5 |
| HC (2) | 4 | | | |
| 4: DFab LC (2) | a-IL-4 kC | 1 | 1 | 1 |
| 5: DFab VLCH1 | a-TSLP | 1 | 1 | 1.5 |
| (1) | CλS | | | |
| Percent peak of | | 40.5% | 55.5% | 70.5% |
| interest | | | | |

Note:
Tx: transfection.
a-IL-4: anti-IL-4.
a-IL-13: anti-IL-13.
sFab: single Fab.
DFab: dual Fab

### Example 33

### Modified Fd (mFd) Design for Single Cell Expression of Five Chains Encoding Tri-Fab-Fc Variants

In a second set of designs to enhance the fidelity of chain pairing, a Fab arrangement referred to as “modified Fd” (mFd) was employed for the domain in the Fab1 position. In this arrangement, the light chain (LC) of Fab1 (outer position) was joined to the amino terminus of the heavy chain (HC) of Fab2 (inner position) via a (Gly)4-Ser linker within an expression vector. This protein chain is referred to as the Dual Fab LC(1)-HC(2). A modified Fd chain was designed to pair with Fab1 LC and it is composed of Fab1 V_{H}, human IgG1-CH1 (SEQ ID NO: 6) and the upper human IgG1 hinge containing Cys at H230 (SEQ ID NO: 102) for interchain disulfide formation with Cys (L214) in Fab1 Kappa constant domain engineered into an expression vector. This design also used electrostatic complementary S1 and S1-reverse mutations to minimize mispairing in the Fab2 and Fab3 arms.

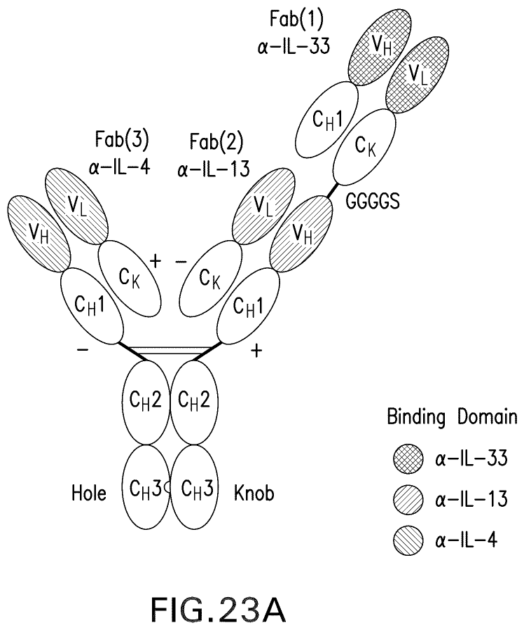

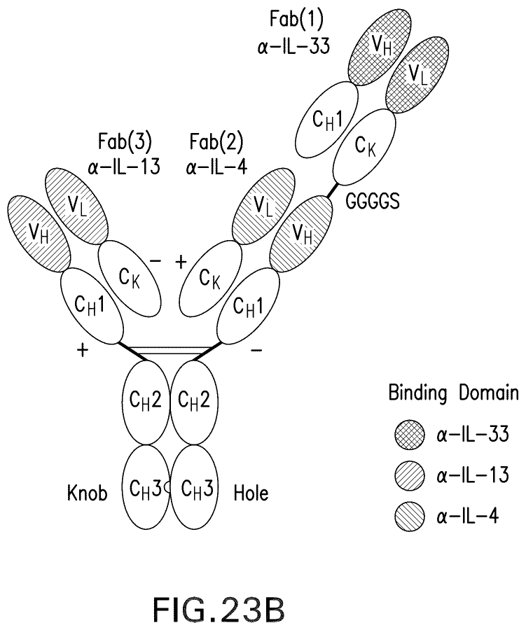

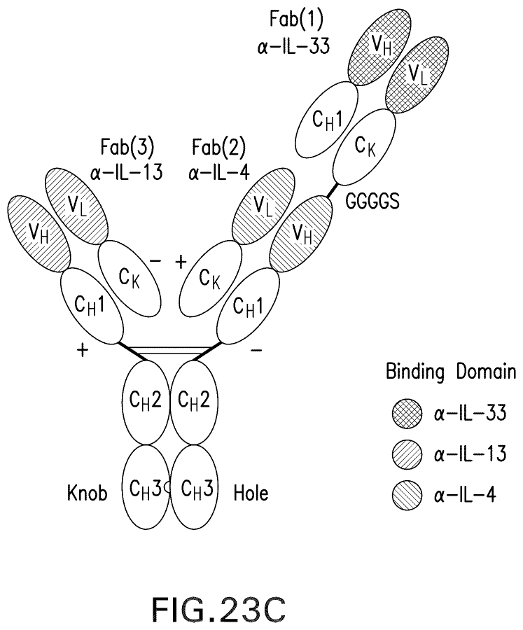

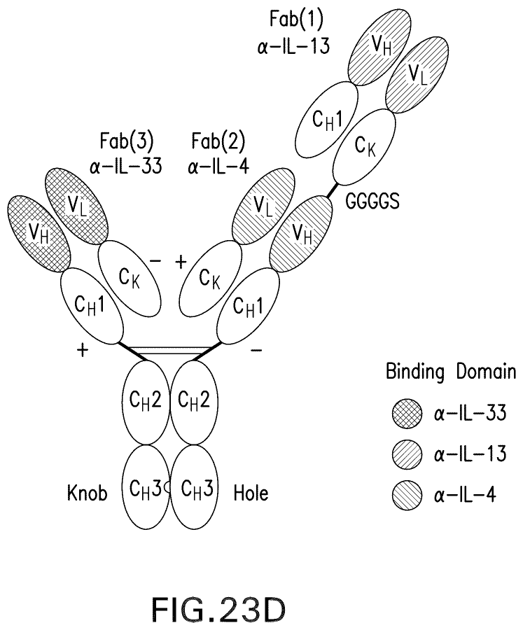

Multiple Tri-Fab-Fc variants made with anti-IL-4, anti-IL-13, and either anti-IL-33, anti-TSLP, or anti-p40 were engineered using the single cell approach where Fc heterodimerization was driven by the Knob-into-Hole mutations and mispairing of light chains was mitigated by both incorporation of electrostatic complementary S1 and S1-reverse mutations in the Fab2/Fab3 positions and inclusion of a modified Fd chain at the Fab1 position. Other design considerations were positioning or geometry of the various Fab binding domains in either the outer (Fab1) or inner (Fab2) positions of the Dual Fab LC(1)-HC(2) chain, as well as the single Fab domain (Fab3). In some instances, the IL-33, TSLP or p40 binding domain was fixed as Fab1 in the outer position of Dual Fab LC(1)-HC(2) chain, while the IL-13 binding domain was located at the inner position (Fab2) and the IL-4 binding domain was located on the SFab arm in the Fab3 position (FIG. 23A), or the IL-4 domain was located in the inner (Fab2) position and the IL-13 domain was located at the Fab1 position and the IL-33, TSLP or p40 binding domain was fixed as the SFab arm in the Fab3 position (FIG. 23D), or the IL-4 domain was located in the inner (Fab2) position and the IL-33, TSLP or p40 binding domain was used as Fab1 and the IL-13 binding domain was located on the SFab arm in the Fab3 position (FIG. 23B). In some instances, the designs in FIGS. 23A and 23B were modified to include the Leucine-Serine (LS) mutations (M(H459)L and N(H465)S, Pfabat numbering) in the IgG1-CH3 domain for half-life extension of the Tri-Fab-Fc molecule, resulting in the designs illustrated in FIGS. 23C and 23D.

TABLE 31
Expression Titer, Analytical Characterization and Thermostability of Tri-Fab-Fc
Variants using Modified Fd (mFd) in Fab1 Position and S1/S1rev Complementary
Mutations at Fab3 and Fab2 Positions

| | | | | | Titer (ProA, | aSEC % | | cGE | | Titer |
|---|---|---|---|---|---|---|---|---|---|---|
| | | | | | mg/L) | main | | % | | (ProA, |
| Tri-Fab-Fc | Fc | | | | Epi2 | peak | | intact | Tm1 | mg/L) |
| Name | format | Fab1 | Fab2 | Fab3 | 93 | (ProA) | MS | NR | (° C.) | ExpiCHO |
| IL413TSLP- | KiH | TSLP- | IL13- | IL4- | 6.82 | 61 | | | | |
| 0005 | | 0001 | 0001 | 0002 | | | | | | |
| | | mFd | S1 | S1rev | | | | | | |
| IL413TSLP- | KiH | TSLP- | IL4- | IL13- | 12.3 | 32 | | | | |
| 0006 | mFd | 0001 | 0002 | 0001 | | | | | | |
| | S1-S1rev | mFd | S1rev | S1 | | | | | | |
| IL413TSLP- | KiH | TSLP- | IL4- | IL13- | n/a | 69.6 | Intact | | 67.91 ± 0.12 | 229.3 |
| 0248 | | 0100 | 0002 | 0001 | | (CHO) | (CHO) | | | |
| | | mFd | S1rev | S1 | | | | | | |
| IL413TSLP- | KiH | TSLP- | IL13- | IL4- | n/a | 73.8 | Intact | | 67.46 ± 0.09 | 175.0 |
| 0250 | | 0100 | 0001 | 0002 | | (CHO) | (CHO) | | | |
| | | mFd | S1rev | S1 | | | | | | |
| IL13433- | KiH | IL33- | IL13- | IL4- | 25.2 | 78.3 | Intact | | | |
| 0005 | | 0232 | 0001 | 0002 | | | | | | |
| | | mFd | S1 | S1rev | | | | | | |
| IL13433- | KiH | IL33- | IL4- | IL13- | 34.3 | 77.6 | Intact | 98.2 | | |
| 0006 | | 0232 | 0002 | 0001 | | | | | | |
| | | mFd | S1rev | S1 | | | | | | |
| IL413p40- | KiH | p40- | IL13- | IL4- | 25.7 | 74.5 | Intact | | 73.45 | n/a |
| 0043 | | 0003 | 0001 | 0002 | | | | | | |
| | | mFd | S1rev | S1 | | | | | | |
| IL413p40- | KiH | p40- | IL4- | IL13- | 31.7 | 83.2 | Intact | | 73.39 | 462 |
| 0044 | | 0003 | 0002 | 0001 | | | | | | |
| | | mFd | S1rev | S1 | | | | | | |
| IL413p40- | KiH | IL13- | IL4- | p40- | n/a | n/a | | | n/a | 77 |
| 0642 | | 0001 | 0002 | 0003 | | | | | | |
| | | mFd | S1rev | S1 | | | | | | |
| IL413p40- | KiH | p40- | IL4- | IL13- | | | | | 70.18 | 119 |
| 0648 | | 0003 | 0157 | 0271 | | | | | | |
| | | mFd | S1rev | S1 | | | | | | |
| IL413TSLP- | KiH | TSLP- | IL4- | IL13- | 3 | | Intact | 99.3 | 70.2 | 85 |
| 0575 | | 0260 | 0157 | 0271 | | | | | | |
| | | mFd | S1rev | S1 | | | | | | |
| IL13433- | KiH | IL33- | IL4- | IL13- | 8.1 | | | | 71.52 | |
| 0606 | | 0224 | 0157 | 0259 | | | | | | |
| | | mFd | S1rev | S1 | | | | | | |
| IL13433- | KiH | IL33- | IL4- | IL13- | 7.1 | 58.2 | | | 71.60 | |
| 0607 | | 0224 | 0157 | 0271 | | | | | | |
| | | mFd | S1rev | S1 | | | | | | |
| IL13433- | KiH | IL33- | IL4- | IL13- | 10 | | | | | |
| 1269 | | 0726 | 1040 | 0001 | | | | | | |
| | | mFd | S1rev | S1 | | | | | | |
| IL13433- | KiH | IL13- | IL4- | IL33- | 172 | 76.8 | Major | 97.1 | 70.92 | |
| 1270 | | 0001 | 1040 | 0726 | | | intact* | | | |
| | | mFd | S1rev | S1 | | | | | | |
| IL13433- | KiH | IL13- | IL4- | IL33- | 195 | 37.7 | Major | | | |
| 1279 | no eng | 0001 | 1040 | 0726 | | | intact** | | | |
| | Cys | mFd | S1rev | S1 | | | | | | |
| | CH3 | | | | | | | | | |

*trace Tri-Fab-Fc minus SFab LC(3)
**trace Tri-Fab-Fc minus SFab LC(3) or DFab LC(2)
n/a: not available

TABLE 32
Neutralizing Activity of Tri-Fab-Fc Variants with Modified Fd (mFd) Fab1 format
and S1/S1rev Complementary Mutations in Fab2 and Fab3

| | | | | | | a-IL-33 | | | | |
|---|---|---|---|---|---|---|---|---|---|---|
| | | | | | a-IL-33 | bioassay | | | | |
| | | | | | bioassay | cys | | a-TSLP | a- | a- |
| | | | | | (WT | mutant | anti-IL12 | TARC | hIL-4 | hIL-13 |
| Tri-Fab-Fc | | | | | IL33) | IL33) | bioassay | bioassay | bioassay | bioassay |
| or Antibody | Fc | | | | IC₅₀ | IC₅₀ | IC₅₀ | (IC₅₀; | IC₅₀, | (IC₅₀, |
| Name | format | Fab1 | Fab2 | Fab3 | (pM) | (pM) | (nM) | pM) | pM) | pM) |
| IL4-0002 | IgG | | | | | | | | 2.39 | |
| IL13-0001 | IgG | | | | | | | | | 5.79 |
| TSLP-0001 | IgG | | | | | | | 18 | | |
| IL413TSLP- | KiH | TSLP- | IL13- | IL4- | | | | | | |
| 0005 | | 0001 | 0001 | 0002 | | | | | | |
| | | mFd | S1 | S1rev | | | | | | |
| IL413TSLP- | KiH | TSLP- | IL4- | IL13- | | | | | | |
| 0006 | | 0001 | 0002 | 0001 | | | | | | |
| | | mFd | S1rev | S1 | | | | | | |
| IL413TSLP- | KiH | TSLP- | IL4- | IL13- | | | | 37 | 5.11 | 15.6 |
| 0248 | | 0100 | 0002 | 0001 | | | | | | |
| | | mFd | S1rev | S1 | | | | | | |
| IL413TSLP- | KiH | TSLP- | IL13- | IL4- | | | | 39 | 3.68 | 102.1 |
| 0250 | | 0100 | 0001 | 0002 | | | | | | |
| | | mFd | S1 | S1rev | | | | | | |
| IL13433- | KiH | IL33- | IL13- | IL4- | ND | 45* | | | 3.2 | 12.5 |
| 0005 | | 0232 | 0001 | 0002 | | | | | | |
| | | mFd | S1 | S1rev | | | | | | |
| IL13433- | KiH | IL33- | IL4- | IL13- | 112 | 158, 40* | | | 10.3 | 9.01 |
| 0006 | | 0232 | 0002 | 0001 | | | | | | |
| | | mFd | S1rev | S1 | | | | | | |
| p40-0003 | IgG | | | | | | 0.286 | | | |
| IL413p40- | KiH | p40- | IL13- | IL4- | | | 2.6 | | 5.10 | 15.1 |
| 0043 | | 0003 | 0001 | 0002 | | | | | | |
| | | mFd | S1 | S1rev | | | | | | |
| IL413p40- | KiH | p40- | IL4- | IL13- | | | 3.03 | | 8.11 | 9.14 |
| 0044 | | 0003 | 0002 | 0001 | | | | | | |
| | | mFd | S1rev | S1 | | | | | | |
| IL413p40- | KiH | IL13- | IL4- | p40- | | | n/a | | n/a | n/a |
| 0642 | | 0001 | 0002 | 0003 | | | | | | |
| | | mFd | S1rev | S1 | | | | | | |
| IL413p40- | KiH | p40- | IL4- | IL13- | | | | | 26.4 | 18.7 |
| 0648 | | 0003 | 0157 | 0271 | | | | | | |
| | | mFd | S1rev | S1 | | | | | | |
| IL413TSLP- | KiH | TSLP- | IL4- | IL13- | | | | 21 | 18.5 | 12 |
| 0575 | | 0260 | 0157 | 0271 | | | | | | |
| | | mFd | S1rev | S1 | | | | | | |
| IL13433- | KiH | IL33- | IL4- | IL13- | ND | ND | | | ND | ND |
| 0606 | | 0224 | 0157 | 0259 | | | | | | |
| | | mFd | S1rev | S1 | | | | | | |
| IL13433- | KiH | IL33- | IL4- | IL13- | ND | 182 | | | 66.9 | 20.3 |
| 0607 | | 0224 | 0157 | 0271 | | | | | | |
| | | mFd | S1rev | S1 | | | | | | |
| IL13433- | KiH | IL33- | IL4- | IL13- | | | | | | |
| 1269 | | 0726 | 1040 | 0001 | | | | | | |
| | | mFd | S1rev | S1 | | | | | | |
| IL13433- | KiH | IL13- | IL4- | IL33- | 12 | ND | | | 16.5 | 7.58 |
| 1270 | | 0001 | 1040 | 0726 | | | | | | |
| | | mFd | S1rev | S1 | | | | | | |
| IL13433- | KiH | IL13- | IL4- | IL33- | 13 | ND | | | 13.7 | 9.13 |
| 1279 | no eng | 0001 | 1040 | 0726 | | | | | | |
| | Cys CH3 | mFd | S1rev | S1 | | | | | | |

*IL-33 neutralization IC₅₀ values determined in the same experiment for IL13433-0005 and IL13433-0006.
ND = not determined.
n/a: not available

Molecules with Fab1 in the mFd format harboring the S1 and S1rev complementary mutations in Fab3 and Fab2, respectively, could be produced (Table 31) and were shown to neutralize all three of their targets (Table 32). Mass spectrometry analysis showed that chain pairing in purified Tri-Fab-Fcs with the mFd format in the Fab1 position plus S1 and S1rev mutations in Fab3 and Fab2was largely correct: purified material corresponding to the Tri-Fab-Fc molecule of IL413p40-0043, IL413p40-0044, IL13433-0005, IL13433-0006 and IL13433-1270 contained little or no mispaired species. This observation is similar to the observations made with molecules harboring V and Cl domain swaps in the Fab1 position, along with S1 and S1rev mutations in Fab3 and Fab2 (Example 31). Further, the importance of the interchain disulfide introduced into the CH3 domains with the KiH heterodimerization mutations for reducing mispaired species is demonstrated by comparison of the percent main peak of interest post-Protein A purification for IL13433-1270, which contains the interchain disulfide (76.8% peak of interest), versus IL13433-1279, which lacks the disulfide (37.7% peak of interest). Taken together, independent engineering strategies focused on enhancing interchain pairing at the Fab1, Fab2, Fab3, and Fc interfaces each contribute to reduction of mispaired species.

The locations of specific binding domains within the set of KiH Tri-Fab-Fc variants using mFd in the Fab1 position, S1rev in the Fab2 position, and S1 in the Fab3 position can influence expression. For example, IL13433-1269 and IL13433-1270 have the same binding domains, but IL13433-1269 has IL13-0001 in the Fab3 position and IL33-0726 in the Fab1 position and expresses 10 mg/L in HEK293, while IL13433-1270 has the same domains in the opposite positions and expresses at 172 mg/L. Similarly, IL413p40-0044 and IL413p40-0642 have the same binding domains, but differ by 6-fold in expression in transient CHO, with IL413p40-0044 (p40-0003 in the Fab 1 position, IL13-0001 in the Fab3 position) expressing >450 mg/L while IL413p40-0642 (p40-0003 in the Fab3 position and IL13-0001 in the Fab1 position) expressing at 77 mg/L. This data set indicates that, for mFd Tri-Fab-Fcs as well as the CkS/CλS/VDS Tri-Fab-Fcs described in EXAMPLE 31, both the specific binding domains and their arrangement in the Tri-Fab-Fc molecule can influence expression titers and bioactivity, illustrating the importance of experimental evaluation.

Thermal stability of Tri-Fab-Fc variants using the mFd format was consistently high among eight molecules tested, with the first melting transition (Tm1) measured by differential scanning calorimetry (DSC) above 67° C. for each molecule (Table 28). Comparison of IL413TSLP-0249 (which has TSLP-0100 in the Fab1 position in CλS format; Tm1 59.9° C.; Table 28) with IL413TSLP-0250, which has the same set of binding domains in the same positions, but using mFd for TSLP-0100 in the Fab1 position, showed an increase in Tm1 to 67.4° C. (Table 31).

As described above for molecules in which Fab1 pairing is driven by VH-VL or Ck/Cl-CH1 position reversals, the Fab at the inner (Fab2) position was able to bind to its target effectively when fused to a Fab in the mFd format in the Fab1 (outer) position. Cell-based activity data (Table 32) indicate that the Fabs in the Fab2 position showed IC₅₀ values typically within 2-3-fold of the same Fab in the Fab3 position (without anything fused to the N terminus). For example, anti-IL4 activity in IL13433-0005 (IL4 in Fab3 position, IC₅₀ 3.2 pM) was similar to that of IL13433-0006 (IL4 in Fab2 position, IC₅₀ 10.3 pM), and anti-IL4 activity in IL413p40-0043 (IL4 in Fab3 position, IC₅₀ 5.1 pM) was similar to that of IL413p40-0043-0044 (IL4 in Fab2 position, IC₅₀ 8.1 pM). Similarly, IL-13 activity in IL13433-0006 (IL-13 in Fab3 position, IC₅₀ 9.0 pM) and IL13433-0005 (IL13 in Fab2 position, IC₅₀ 12.5 pM) were very close to one another, as were IL-13 activities in IL413p40-0044 (IL13 in Fab3 position, IC₅₀ 9.1 pM) and IL413p40-0043 (IL13 in Fab2 position, IC₅₀ 15 pM). The one exception was IL413TSLP-0250, which had IL13-0001 in the Fab2 position and displayed significantly weaker IL-13 neutralizing activity (IC₅₀ 102.1 pM) than did its counterpart with IL13-0001 in the Fab3 position, IL413TLSP-0248 (IC₅₀ 15.6 pM). Thus, the degree to which activity of the Fab2 domain was affected by its position was influenced by the specific binding domain in the Fab1 position.

### Example 34

### Transient Expression Levels of Anti-IL-4/IL-13/IL-33 Tri-Fab-Fc Molecules with Varied Composition and Geometry of Binding Domains

To assess the impact of Tri-Fab-Fc geometry and individual domain sequences on the expression of five independent protein chains within a single cell, a set of anti-IL-4/13/33 Tri-Fab Fc variants designed using the single cell approach were produced using the transient HEK-293 expression system and compared to the expression of the component binding domains in IgG format. Transient expression was performed with PEI-MAX transfection of DNA encoding the five chains comprising the Tri-Fab-Fc in the Expi293F™ host cells using the manufacturer's recommended protocol. Conditioned medium was harvested 5 days post-transfection or when cell viability dropped below 60%. Expression titers for conditioned medium produced either using transient or stable expression systems were determined using the following method. Conditioned medium (0.9 mL) filtered through 0.2 μm membrane was passed through 1 mL Protein A resin (Cytiva, Westborough, MA) pre-equilibrated with PBS-CMF (137 mM NaCl, 2.7 mM KCl, 8.1 mM Na₂HPO₄, 2.7 mM KH₂PO₄, pH 7.2) in an Agilent 1200 Series HPLC Gradient System (Agilent Technologies, Santa Clara, CA). The column was washed with 10 mL of PBS buffer before the protein was eluted using 100% step of 150 mM Glycine, 40 mM NaCl, pH 3.5. The area of elution peaks in A280 was integrated and converted into protein amount through a linear standard curve generated with purified human IgG antibody control adjusted by extinction coefficient.

The results showed that transient expression titers of the Tri-Fab-Fcs varied from 7-172 mg/L. The designs used for IL13433-0005 and IL13433-0006 exhibited a modest yield of Tri-Fab-Fc with expression titers of 25.2 mg/L and 32.0 mg/L, respectively. However, when the binding domains IL4-0157, IL33-0224 and either IL13-0259 or IL13-0271 were engineered into the same format used for IL13433-0006, thus generating IL13433-0606 and IL13433-0607, respectively, the resultant expression titers were unexpectedly much lower (Table 33). This was surprising since the IL4-0157, IL33-0224, IL13-0259 and IL13-0271 variants produced in a standard IgG antibody format all displayed expected high transient expression titers (Table 33). These results indicate that small amino acid changes introduced into the IL-4, IL-13 and IL-33 binding domains to reduce in silico predicted non-germline T-cell epitopes and sequence liabilities in CDR regions significantly impacted the amount of intact Tri-Fab-Fc secreted from the cells. Additionally, the impact of positioning or geometry of the individual binding domains is evident when comparing the Tri-Fab-Fc variants IL13433-1269 to IL13433-1270 (FIG. 14C versus 14D) since both were engineered with identical binding domains, however IL13433-1270 exhibited significantly higher (˜17-fold) transient expression versus IL13433-1269 (Table 17). Further, the transient expression titer of 172 mg/L for Tri-Fab-Fc IL13433-1270 was in the range of expression titers obtained for a typical antibody with a favorable expression profile.

Table 33 also illustrates that an additional Tri-Fab-Fc format could be produced using an alternative chain pairing strategy. The binding domains used in IL13433-0005 were placed in the same positions in IL13433-0021 (IL33-0232 in Fab1; IL13-0001 in Fab2; and IL4-0002 in Fab3), but in IL13433-021, IL13-0001 (Fab2) chain pairing was driven by use of a C kappa swap, (FIG. 20e) instead of S1. Similarly, in IL13433-021, IL33-0232 (Fab1) chain pairing was driven by use of S1 mutations instead of the mFd arrangement found in IL13433-0005. Table 33 illustrates that protein yields were similar for both Tri-Fab-Fc molecules. In addition, similar expression was observed for IL13433-0022 (FIG. 20f), a molecule designed to increase the flexibility of the linker between the VL and CH1 domain of the IL13-0001 Fab2 in the CκS format by adding two serine residues to mimic the conserved C terminal two amino acids (SS) for all human J- segments prior to the alanine residue at the first position in human IgG1. Further, the IL-13 neutralization activity assessed using the IL-13 induced CD23 upregulation bioassay was slightly more potent for IL13433-0022 with the SS elbow in the IL-13 binding domain relative to IL13433-0021, specifically 17.46 pM versus 27.19 pM.

TABLE 33
Transient Expression Titers from Expi293 ™ Cell Expression System for
Single Cell Produced Tri-Fab-Fc and Component Domains in IgG Format

| | Fc | Binding Domain/Orientation | | | Titer |
|---|---|---|---|---|---|
| Protein name | format | Fab1 | Fab2 | Fab3 | [mg/L] |
| IL4-0002 | IgG | | | | 313.7 |
| IL4-0157 | IgG | | | | 382.3 |
| IL13-0001 | IgG | | | | 204.9 |
| IL13-0259 | IgG | | | | 415.6 |
| IL13-0271 | IgG | | | | 376.4 |
| IL33-0232 | IgG | | | | 203.2 |
| IL33-0224 | IgG | | | | 285.6 |
| IL13433-0005 | KiH | IL33-0232 mFd | IL13-0001 S1 | IL4-0002 S1 | 25.2 |
| | | | | rev | |
| IL13433-0006 | KiH | IL33-0232 mFd | IL4-0002 S1Rev | IL13-0001 | 34.3 |
| | | | | S1 | |
| IL13433-0021 | KiH | IL33-0232 S1 | IL13-0001 CKS | IL4-0002 S1 | 30.8 |
| | | | | rev | |
| IL13433-0022 | KiH | IL33-0232 S1 | IL13-0001 CkS with | IL4-0002 S1 | 32.5 |
| | | | SS-elbow | rev | |
| IL13433-0606 | KiH, LS | IL33-0224 mFd | IL4-0157 S1Rev | IL13-0259 | 8.1 |
| | | | | S1 | |
| IL13433-0607 | KiH, LS | IL33-0224 mFd | IL4-0157 S1Rev | IL13-0271 | 7.1 |
| | | | | S1 | |
| IL13433-1269 | KiH, LS | IL33-0726 mFd | IL4-1040 S1Rev | IL13-0001 | 10 |
| | | | | S1 | |
| IL13433-1270 | KiH, LS | IL13-0001 mFd | IL4-1040 S1Rev | IL33-0726 | 172 |
| | | | | S1 | |

CH1-Cκ swap = Fab has CH1 and Kappa constant domains exchanged; KiH = Knob-into-Hole; LS = Leu/Ser half-life extension mutations; mFd = modified Fd chain; S1-S1rev = electrostatic complementary S1 and S1-reverse mutations; SS elbow = Ser-Ser amino acids inserted between V_{L} and CH1.

### Example 35

### Stable CHO Expression of Anti-IL-41IL-13/IL-33 Tri-Fab-Fc Variants Produced from Single Cell Lines

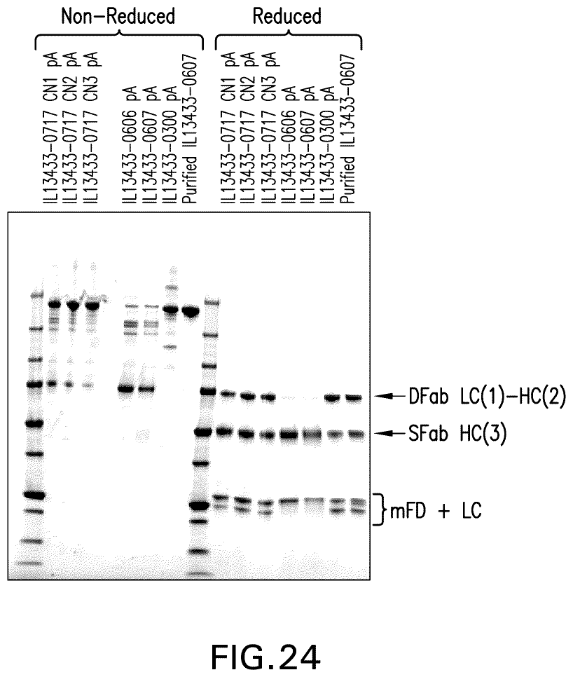

Stable CHO expression was evaluated for select anti-IL-4/IL-13/IL-33 Tri-Fab-Fc variants engineered using the single cell approach to gauge feasibility for stable cell line development. The five unique chains comprising the Tri-Fab-Fc were sub-cloned into a CHO SS1 vector (Lonza) using the following arrangement: Modified Fd(i)→DFab LC(2)→DFab LC(i)-HC(2)→SFab LC(3)→SFab HC(3). Modifications to prepare for stable CHO expression were to include the C-terminal Lysine (CTK) onto the heavy chain of the Tri-Fab-Fc variants where it had been omitted for transient HEK-293 expression. In one instance, addition of the CTK to the prototype IL3433-0006 variant generated the Tri-Fab-Fc variant IL3433-0300 (FIG. 23C). The LS mutations, specifically M(H459)L and N(H465)S (Pfabat numbering), are incorporated to increase serum half-life of therapeutics to offer increased dosing flexibility. The LS mutations were engineered into IL3433-0300, yielding the IL3433-0717 Tri-Fab-Fc variant as a direct comparison to ensure that these modifications to the protein did not alter the expression titer level. Three independent stable CHO pools were generated for each Tri-Fab-Fc variant, and expression titers were determined from 600 mL shake flask cultures. In some instances, the three independent pools were combined prior to determining expression titers resulting in a “combined pool”. The results indicate that inclusion of LS mutations does not significantly alter transient expression level of the Tri-Fab-Fc (Table 34). However, minimal amino acid sequence substitutions significantly impacted expression titers of IL13433-0606 and IL13433-0607, which differed only by 4 amino acids in the TriFab of the IL13 domain (Table 4). Additionally, reduced Stain Free SDS-PAGE assessment of equivalent amounts of the Protein A purified protein from pooled conditioned medium indicated minimal secretion of the IL13433-0606 and IL13433-0607 DFab LC(1)-HC(2) protein chain as compared to the equivalent chains from IL13433-0300 (-LS) and IL13433-0717 (+LS), FIG. 24. Analytical Size Exclusion Chromatography (aSEC) results (Table 34) correlated with observations from non-reduced Stain-free SDS-PAGE assessment of the Protein A purified protein where minimal peak-of-interest (POI) was observed for IL13433-0606 and IL13433-0607 variants relative to IL13433-0300 and IL13433-0717. These combined results also suggest that inclusion of LS mutations does not alter expression titer and secretion of Tri-Fab-Fc from the cell but that small sequence changes introduced to the IL4-0157 V_{H} and IL13-0271 V_{L} domains within the DFab LC(1)-HC(2) chain can lead to dramatic changes in secretion of intact Tri-Fab-Fc, likely through reduced secretion of the DFab LC(1)-HC(2) chain.

TABLE 34
Stable CHO Expression Titers and Analytical SEC
for Single Cell Produced Tri-Fab-Fc Variants
using Stable CHO SSI Expression System.

| | | Binding Domain/Orientation | | | | % POI |
|---|---|---|---|---|---|---|
| | | Fab1 | Fab2 | Fab3 | Titer | via |
| Tri-Fab- | Fc | (mFd | (S1rev | (S1 | [mg/ | analytical |
| Fc | format | format) | format) | format) | L] | SEC |
| IL13433- | KiH | IL33-0232 | IL4-0002 | IL13-0001 | 376 | 70.94 |
| 0300 | | | | | | |
| IL13433- | KiH, LS | IL33-0232 | IL4-0002 | IL13-0001 | 102 | 56.69, |
| 0717 | | | | | | 69.82, |
| | | | | | | 79.53* |
| IL13433- | KiH, LS | IL33-0224 | IL4-0157 | IL13-0259 | 72 | 11.17 |
| 0606 | | | | | | |
| IL13433- | KiH, LS | IL33-0224 | IL4-0157 | IL13-0271 | 9 | 13.69 |
| 0607 | | | | | | |

KiH = Knob-into-Hole;
LS = CH3 mutations for half-life extension;
mFd = modified Fd chain;
pA = Protein A;
pooled material = combined from three independent stable CHO pool 600 mL
shake flask cultures;
S1-S1rev = electrostatic complementary S1 and S1-reverse mutations.
*3 independent stable CHO pools in 600 mL shake flask cultures

### Example 36

### Engineering Tri-Fab-Fc Variants Using an RRR-EEE Charge-Based (CB) Fc Heterodimerization Approach

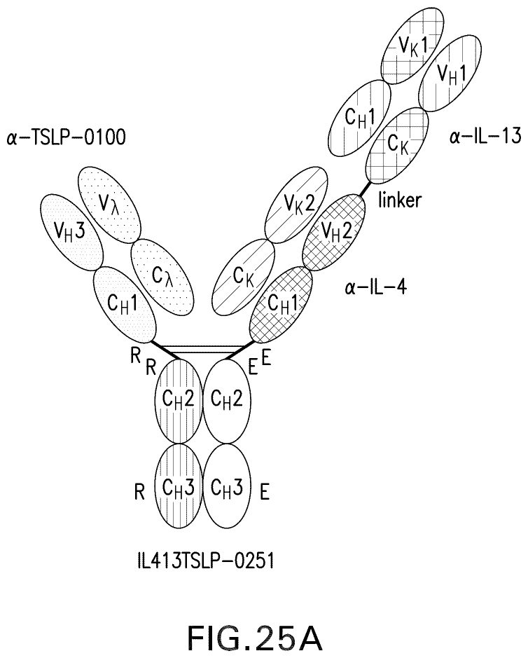

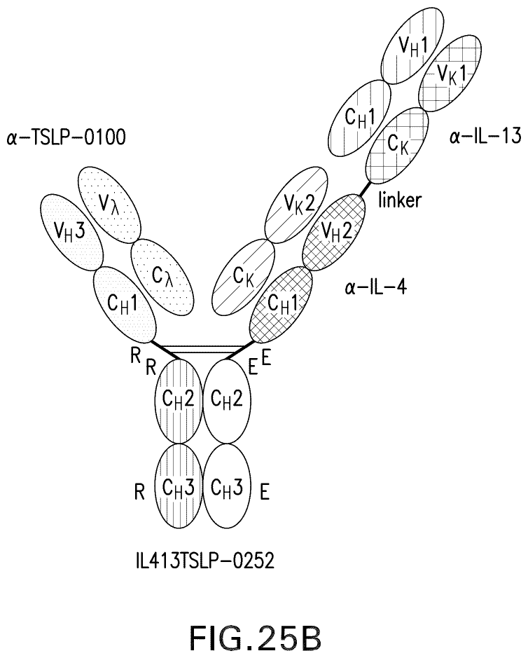

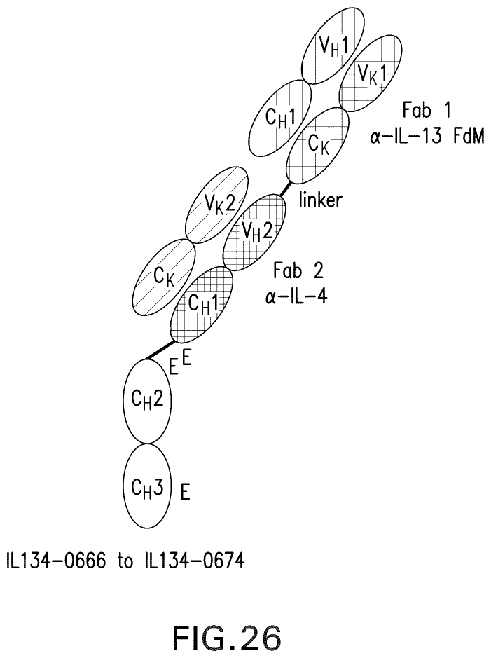

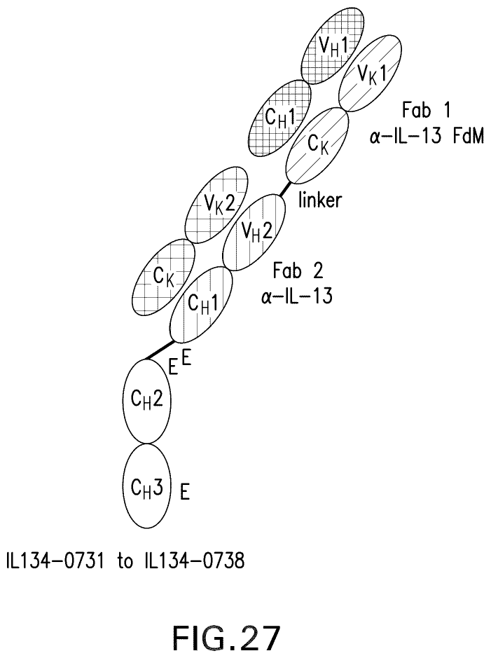

To elucidate the unexpected observations of low transient HEK-293 and stable CHO expression titers for IL13433-0606 and IL13433-0607 Tri-Fab-Fcs and to facilitate production of systematically varied Tri-Fab-Fc molecules, an alternative Tri-Fab-Fc design was employed. The charge-based (CE) heterodimerization approach, described by Strop et al. (12) in the context of bispecific antibody generation, allows separate expression of the two portions of an asymmetric heterodimeric molecule and subsequent joining to form the heterodimer. This technology relies on placement of oppositely charged residues, arginine and glutamic acid, in equivalent positions in the hinge and CH3 domains of two IgGs. These IgGs are expressed and purified separately as homodimers, but then are mixed and treated with a reducing agent to break the disulfide bonds between the two heavy chains. The positioning of opposite charges at the Fc interface leads to preferential pairing of one heavy chain from each of the two starting IgGs, and when the mixture is then re-oxidized, disulfide bonds re-form, yielding a mixture that is predominantly heterodimeric. This method was adapted to the formation of Tri-Fab-Fc antibodies by separate expression of the dual Fab component in one cell and the single Fab component in another. For example, in the molecules shown in FIG. 25, the dual Fab arm composed of the anti-IL-13 binding domain in the Fab1 or outer position and anti-IL-4 in the Fab2 or inner position is expressed in one cell, while the second cell produces the SFab arm, with the anti-TSLP Fab in the Fab3 position (FIG. 25). In this instance, three positively charged residues (referred to as RRR) were introduced into the human IgG1 constant region: two mutations D(H232)R and P(H241)R into the hinge and K(H440)R into the CH3 region of the single Fab chain. The three opposite negative charge-paired mutations incorporated into human IgG1 constant region of the dual Fab arm were D(H232)E and P(H241)E into the hinge region and L(H391)E into the CH3 domain, referred to collectively as EEE. In this instance, correct pairing of light chain of Fab 3 with its corresponding heavy chain was achieved without the use of any mutations, since only one heavy chain and one light chain were expressed in the cell. The correct pairing of chains in the dual Fab arm was achieved by using either the CkS or mFd designs for Fab1, described above, while Fab 2 did not harbor any mutations.

cDNAs encoding each chain were cloned into expression vectors. Two cDNA vectors for the single Fab RRR arm encoding anti-TSLP SFab HC(3) chain and SFab LC(3) lambda chain or three cDNA vectors for dual Fab EEE arm encoding anti-IL-13/IL-4 dual Fab long chain and two associated short chains were separately transfected into ExpiCHO-S cells or Expi293F™ cells according to the manufacturer's protocol (Thermo Fisher Scientific). The conditioned media of homodimer RRR arm and EEE arm were harvested on day 7 and captured with 5 mL HiTrap MabSelect™ SuRe™ LX (GE Healthcare Life Sciences). The eluted antibodies were neutralized with 2M HEPES pH8.0. Heterodimerization of Tri-Fab-Fc molecule was prepared by incubation of a mixture of an equimolar ratio of the RRR and EEE arms with 1 mM Glutathione (GSH) at 37° C. for 6 hours (reducing step) followed by dialysis in cold PBS overnight (oxidization step). The purification was performed on an AKTA Explorer (Agilent, Santa Clara, CA) with first loading to an anion exchange column Mono Q 5/50 GL (GE Healthcare; Ser. No. 17/516,601; Lot: 10265412). The final Tri-Fab-Fc was further purified by Superdex 200 (GE Healthcare Life Sciences, Piscataway, N.J) if needed. The quality of the Tri-Fab-Fcs was examined by analytic SEC (Table 35) and LC/MS (intact and reduced subunit analysis). The actual molecular weights (MWs) of each Tri-Fab-Fc, including intact and subunits, were matched to theoretical MWs (MS data not shown).

Biophysical properties were evaluated via analytical size-exclusion chromatography (aSEC), DSC, heat-forced aggregation propensity, low pH hold, and non-specificity evaluation (AC-SINS and polyreactivity). For HPLC aSEC analysis, samples were run in a YMC-Pack Diol-200 SEC column using a buffer containing 20 mM sodium phosphate and 400 mM NaCl at pH 7.2. Injection volumes of 5 μL molecular weight standards (MWS), 25 μL control IgG1 antibody, and 50 μg per sample were used, drawing and ejecting both at 150 μL/minute. The retention time and peak width of the main peak as well as the areas and percent areas of the main, LMMS, and HMMS peaks were recorded. The areas of the main, LMMS, and HMMS peaks were used to calculate the mass recovery and percent mass recovery of each sample.

TABLE 35
Expression Titer, aSEC and Thermostability of Tri-Fab-Fc Molecules using
Charge-Based Heterodimerization

| | | | | | | | Titer | aSEC % | |
|---|---|---|---|---|---|---|---|---|---|
| | | | | | Titer (ProA, | aSEC % | (ProA, | main | |
| | | | | | mg/L) | main peak | mg/L) | peak | |
| Tri-Fab- | | | | | Epi293 | (ProA) | Epi293 | (ProA) | DSC |
| Fc | Fc | | | | single | single | dual Fab | dual Fab | Tm1 |
| name | format | Fab1 | Fab2 | Fab3 | Fab arm | Fab arm | arm | arm | (° C.) |
| IL413TS | CB | IL13- | IL4- | TSLP | 115.2 | 97.6 | 246.3 | 95.3 | 70.24 |
| LP-0251 | EEE- | 0001 | 0002 | (0100) | *CHO | | *CHO | | |
| | RRR | (CkS) | (native | (native | | | | | |
| | | | Fab) | Fab) | | | | | |
| IL413TS | CB | IL13- | IL4- | TSLP | 115.2 | 97.6 | 347.8 | 95.5 | 70.50 |
| LP-0252 | EEE- | 0001 | 0002 | (0100) | *CHO | | *CHO | | |
| | RRR | (mFd) | (native | (native | | | | | |
| | | | Fab) | Fab) | | | | | |
| IL13433- | CB | IL13- | IL4- | IL33- | 263 | | 155 | | 69.84 |
| 1042 | EEE- | 0001 | 0749 | 0726 | | | | | |
| | RRR | (mFd) | (native | (native | | | | | |
| | | | Fab) | Fab) | | | | | |
| IL413p4 | CB | IL13- | IL4- | p40- | 23.5 | 97.8 | 157 | 45.7 | 71.7 |
| 0-0698 | EEE- | 0001 | 0749 | 0003 | | | | | |
| | RRR | (mFd) | (native | (native | | | | | |
| | | | Fab) | Fab) | | | | | |
| IL413p4 | CB | IL13- | IL4- | p40- | 23.5 | 97.8 | 26 | 51.4 | 70.99 |
| 0-0700 | EEE- | 0001 | 1040 | 0003 | | | | | |
| | RRR | (mFd) | (native | (native | | | | | |
| | | | Fab) | Fab) | | | | | |

*Produced transiently in ExpiCHO cells; others produced in HEK293

TABLE 36
Bioactivity of Tri-Fab-Fc Molecules using EEE/RRR Charge-Based
Heterodimerization

| | | | | | a-IL-33 | anti- | anti- | a-TSLP | | |
|---|---|---|---|---|---|---|---|---|---|---|
| | | | | | bioassay | IL12 | IL23 | TARC | a- | a-hIL- |
| Tri-Fab- | Tri- | | | | (WT) | bioassay | bioassay | bioassay | hIL-4 | 13 |
| Fc | Fab-Fc | | | | IC₅₀ | IC₅₀ | IC₅₀ | IC₅₀ | IC₅₀ | IC₅₀ |
| name | format | Fab1 | Fab2 | Fab3 | [pM] | [nM] | [nM] | [pM] | [pM] | [pM] |
| IL413T | CB | IL13- | IL4- | TSLP | | | | 58 | 6.11 | 13.7 |
| SLP- | EEE- | 0001 | 0002 | (0100 | | | | | | |
| 0251 | RRR | (CKS) | (native | (native | | | | | | |
| | | | Fab) | Fab) | | | | | | |
| IL413T | CB | IL13- | IL4- | TSLP | | | | 42 | 3.65 | 11.2 |
| SLP- | EEE- | 0001 | 0002 | (0100) | | | | | | |
| 0252 | RRR | (mFd) | (native | (native | | | | | | |
| | | | Fab) | Fab) | | | | | | |
| IL13433- | CB | IL13- | IL4- | IL33- | 28 | ND | ND | ND | 7.1 | 12.4 |
| 1042 | EEE- | 0001 | 0749 | 0726 | | | | | | |
| | RRR | (mFd) | (native | (native | | | | | | |
| | | | Fab) | Fab) | | | | | | |
| IL413p4 | CB | IL13- | IL4- | p40- | | 1.56E−10 | 2.10E−10 | | 12.2 | 11.75 |
| 0-0698 | EEE- | 0001 | 0749 | 0003 | | | | | | |
| | RRR | (mFd) | (native | (native | | | | | | |
| | | | Fab) | Fab) | | | | | | |
| IL413p4 | CB | IL13- | IL4- | p40- | | 7.71E−10 | 2.03E−09 | | 13.2 | 12.24 |
| 0-0700 | EEE- | 0001 | 1040 | 0003 | | | | | | |
| | RRR | (mFd) | (native | (native | | | | | | |
| | | | Fab) | Fab | | | | | | |

ND: not determined

Tri-Fab-Fc molecules using the EEE-RRR charge-based heterodimerization strategy could be produced and were shown to neutralize all three of their targets. Notably, the production of the two parts of each Tri-Fab-Fc molecule in two different cells enabled successful Trispecific generation using mutations in only one of the three Fab domains, in addition to hinge and CH3 mutations, to drive correct chain pairing. Tri-Fab-Fc molecules built with the Fab1 domain in either modified Fd (mFd) or C kappa swapped (CkS) format (with Fab2 and Fab3 using native sequences for chain pairing) were successfully produced and demonstrated to be functional.

The thermal stability of the Tri-Fab-Fc molecules tested in Table 35 was uniformly high, >70° C. for the four molecules evaluated. Intact mass spectrometry analysis of IL413TSLP-0251 (CkS format) and IL413-TSLP-0252 (mFd format) showed that the majority of the purified material is the correctly assembled Tri-Fab-Fc, with no mispaired species detected. The samples contained low levels of the SFab arm and the Dual Fab arm, suggesting that the samples had been incompletely oxidized during bispecific assembly. Importantly, the absence of mispairing indicates that despite the absence of mutations to force pairing in the Fab2, either the CkS or the mFd modification in Fab1 is sufficient to drive specific pairing of both Fab1 and Fab2 during co-expression of the three chains that make up the Dual Fab arm.

The IL13-0001 domain was tested for neutralization potency in the Fab1 position, in both mFd format (in the Tri-Fab-Fc IL413TSLP-0252) and in CkS format (in the otherwise-identical Tri-Fab-Fc IL413TSLP-0251), and in both formats had highly similar activity (IC₅₀ 11.2 and 13.7 pM, respectively; Table 36). Likewise, the IL4-0002 domain in Fab2 position in these two TSLP Tri-Fab-Fc variants had similar activity (IC₅₀ 3.65 and 6.11 pM, respectively; Table 36). While the other molecules in Table 36 were tested in independent assays and cannot be compared directly, the consistent high potency of neutralization observed for the IL-13 and IL-4 Fabs on the dual Fab arm suggests that in the CB heterodimer format, there is little or no influence of the binding domain in the Fab3 position on the functions of Fab1 and Fab2, and that therefore the parts of the molecule that are produced separately can be engineered separately.

Tri-Fab-Fc trispecifics were evaluated through DSC and heat forced aggregation propensity. Heat forced aggregation propensity was used to determine the protein stability under heat stressed conditions. The method was described previously by Fennel B J et al (5). In brief with some modifications, 20 uL protein samples at 1 mg/mL in PBS were placed in a 96-well optical reaction plate (Applied Biosystems), covered with 30 μL of mineral oil (Sigma-Aldrich), and sealed with an adhesion film (Applied Biosystems). The plate was placed in a PCR block heater (Bio-Rad C1000 Touch Thermal Cycler) with a constant temperature gradient ranging from 40 to 64° C. After 24 h incubation, 10 μL of each of the heated samples, as well as a control sample kept at 4° C., were analyzed on an Agilent 1200-series HPLC (Agilent Technologies), with a QC-PAK-GFC300 column (Tosoh Bioscience LLC), and PBS as running buffer. For each sample, the percent aggregate was calculated as the ratio of remaining antibody molecule peak area at each temperature to peak area of the 4° C. control sample. A plot of % HMMS vs. temperature was made for each antibody and compared to historical data of clinical mAbs to determine the aggregation propensity.

Among three TSLP Tri-Fab-Fc molecules, two trispecifics with identical binding domains and with Fab1 in mFd format (IL413TSLP-0248 and -0250) exhibited higher Tm1 and improved aggregation propensity compared to the equivalent Tri-Fab-Fc IL413TSLP-0249 with Fab1 in CλS format (Table 37). Furthermore, the charge-based Tri-Fab-Fcs IL413TSLP-0251 and IL413TSLP-0252, with IL13-0001 in Fab1 position and IL4-0002 in Fab2 position, showed further improvement in thermal stability over the knobs-into-holes Tri-Fab-Fcs with the same binding domains in different positions (Table 37). Hence, both chain-pairing strategy and domain position impact biophysical properties of Tri-Fab-Fcs that use the same set of binding domains, underscoring the need for experimental identification of optimal arrangements.

TABLE 37
Thermostability and Aggregation Propensity Comparison

| | | | | | pH Hold | | |
|---|---|---|---|---|---|---|---|
| | | | | | at 3.4 | | |
| Tri-Fab- | Fc | | | | ↑ % H | Tm1 | Aggregation |
| Fc Name | format | Fab1 | Fab2 | Fab3 | MMS | (° C.) | Propensity |
| IL413TSL | KiH | TSLP- | IL4-0002 | IL13-0001 (S1) | 11.5 | 67.91 ± 0.12 | low |
| P-0248 | | 0100 | (S1Rev) | | | | |
| | | (mFd) | | | | | |
| IL413TSL | KiH | TSLP- | IL13-0001 | IL4-0002 | 13.2 | 67.46 ± 0.09 | low |
| P-0250 | | 0100 | (S1) | (S1Rev) | | | |
| | | (mFd) | | | | | |
| IL413TSL | KiH | TSLP- | IL4- | IL13-0001 (S1) | 15.9 | 59.90 ± 0.02 | high |
| P-0249 | | 0100 | 0002(S1Rev) | | | | |
| | | (CλS) | | | | | |
| IL413TSL | CB | IL13- | IL4-0002 | TSLP (0100) | 11.1 | 70.24 ± 0.40 | low |
| P-0251 | EEE- | 0001(CkS) | (native Fab) | (native Fab) | | | |
| | RRR | | | | | | |
| IL413TSL | CB | IL13- | IL4-0002 | TSLP (0100) | 10.0 | 70.50 ± 0.01 | low |
| P-0252 | EEE- | 0001(mFd) | (native Fab) | (native Fab) | | | |
| | RRR | | | | | | |

### Example 37

### Optimization of Dual Fab IL-4/IL-13 EEE Arm with mFd Designs and Production Via Transient Expi293™ Expression System

To develop a robust anti-IL-4/IL-13 dual Fab arm using the EEE-RRR charge-based (CB) Fc heterodimerization approach, several design elements of an anti-IL-13/anti-IL-4 dual Fab arm were systematically varied: position of the IL-4 and IL-13 domains in the dual Fab arm sequences of the VH and VL of each binding domain; and the use of CκS and mFd to drive pairing of the Fab1. Such a dual Fab arm is advantageous in that it could be applied to produce multiple different anti-IL-4/IL-13 Tri-Fab-Fc variants that differ only by the third unique functional arm. In addition, the lower complexity of expression logistics of the CB approach facilitated studies to understand which CDR sequences were responsible for unexpectedly low transient HEK-293 and stable CHO expression titers for two highly similar KiH TriFab Fcs, IL13433-0606 and IL13433-0607 (Table 34). These two TriFab-Fc molecules were identical in their Fab1 (IL33-0224, mFd format) and Fab2 (IL4-0157, S1 rev) domains and differed in their Fab3 domains (IL13-0259 in IL13433-0606 and IL13-0271 in IL13433-0607). Further, IL13-0259 and IL13-0271 have identical VH, but different VL with notable amino acid differences in CDRL2 for IL13-0271. Variant VH and VL domain sequences in the IL-4 and IL-13 Fabs were systematically combined to identify sequences associated with poor expression in both KiH and CB TriFab-Fcs.

Various dual Fab arms chains and the associated light chain or mFdL-CH1 chains were engineered into an expression vector, transiently expressed in the Expi293F™ host cells and expression titers for the resultant conditioned medium were determined as previously described with the Protein A column. Capillary gel electrophoresis (cGE; LabChip, Perkin Elmer) was used to gauge the percentage of desirable half/homodimer and non-desirable non-half/homodimer percent missing chain after Protein A capture.

TABLE 38
Transient Expression Titers and cGE Analysis of Dual Fab Arms Harboring IL-4
and IL-13 Binding Domains with Fab1 in mFd format

| | | | | IL-13 Fab1 (outer); IL-4 Fab2 | | | IL-4Fab1 (outer); IL-13 Fab2 | | |
|---|---|---|---|---|---|---|---|---|---|
| | | | | (inner) | | | (inner) | | |
| | | | | | Transient | | | Transient | |
| | | | | Dual Fab | Expression | % | Dual Fab | Expression | % |
| IL-13 Fab | | IL-4 Fab | | EEE Arm | Titer | Half/ | EEE Arm | Titer | Half/ |
| VL | VH | VL | VH | Name | (mg/L) | Homodimer | Name | (mg/L) | Homodimer |
| IL13-0271 | IL13- | IL4-0157 | IL4-0157 | IL134- | 14 | 91.8 | IL134- | 87.6 | 77.4 |
| | 0259 | | | 0666 | | | 0731 | | |
| IL13-0259 | IL13- | IL4-0157 | IL4-0157 | IL134- | 15 | 89.4 | IL134- | 186.3 | 78.2 |
| | 0259 | | | 0667 | | | 0732 | | |
| IL13-0001 | IL13- | IL4-0157 | IL4-0157 | IL134- | 23 | 92.9 | IL134- | 70.6 | 79.8 |
| | 0001 | | | 0669 | | | 0734 | | |
| IL13-0259 | IL13- | IL4-0157 | IL4-0002 | IL134- | 55 | 82.3 | IL134- | 90.2 | 74.8 |
| | 0259 | | | 0670 | | | 0735 | | |
| IL13-0001 | IL13- | IL4-0157 | IL4-0002 | IL134- | 96 | 94.7 | IL134- | 88.9 | 79.3 |
| | 0001 | | | 0672 | | | 0737 | | |
| IL13-0271 | IL13- | IL4-0157 | IL4-0002 | IL134- | 32 | 85.6 | IL134- | 52.2 | 76.7 |
| | 0259 | | | 0673 | | | 0738 | | |
| IL13-0259 | IL13- | IL4-0157 | IL4-0157 | | | | IL134- | 92 | 78 |
| | 0001 | | | | | | 0733 | | |
| IL13-0259 | IL13- | IL4-0157 | IL4-0002 | | | | IL134- | 90.4 | 77.7 |
| | 0001 | | | | | | 0736 | | |
| IL13-0001 | IL13- | IL4-0157 | IL4-0157 | IL134- | 16 | 88.9 | | | |
| | 0259 | | | 0668 | | | | | |
| IL13-0001 | IL13- | IL4-0157 | IL4-0002 | IL134- | 66 | 93 | | | |
| | 0259 | | | 0671 | | | | | |
| IL13-0001 | IL13- | IL4-0002 | IL4-0002 | IL134- | 184 | 92.8 | | | |
| | 0001 | | | 0674 | | | | | |

Protein A column determined titer of transiently expressed conditioned medium and analysis of purified Fab1 anti-IL-13/Fab 2 anti-IL-4 dual Fab arms by cGE.
Note:
IL13-0259 and IL13-0271 have identical V_{H}

The set of dual Fab arms designed to test two variables, sequence variations of the IL4 and IL13 binding domains and the positions of the IL4 and IL13 binding domains in the dual Fab arm, was examined for protein titer and the size of expressed species following Protein A purification. Within this set, all constructs had a high proportion of molecules of the intended sizes in the pool of material purified on Protein A, either homodimers of the dual Fab arm or half-molecules consisting of one dual Fab arm (dFab and associated LC and mFd). The constructs with anti-IL4 in the Fab1 (outer) position and anti-IL-13 in the Fab2 (inner) position ranged from 75-79% homodimer/half molecule, while the constructs in the opposite orientation had consistently slightly higher proportions of homodimer/half molecule (82-94%; Table 38). This pattern was evident in all of the 6 examples in which both orientations were examined.

Expression levels of these dual Fab arms, as measured by protein captured on Protein A, varied considerably, and appeared to be associated with specific sequences in specific locations. For example, when the anti-IL4 Fab was in the Fab2 (inner) position, constructs containing both the VH and VL domains of IL4-0157 had consistently low titers compared to constructs containing the VH domain of IL4-0002 and the VL domain of IL4-0157 (Table 38): IL134-0666 (Fab2 of IL4-0157 VH, IL4-0157 VL; Fab1 of IL13-0259 VH, IL13-0271 VL) had a titer of 14 mg/L, compared to 32 mg/L for IL134-0673, which was identical except that it contained IL4-0002 VH instead of IL4-0157 VH. Similarly, IL134-0667 (Fab2 of IL4-0157 VH, IL4-0157 VL; Fab1 of IL13-0259 VH, IL13-0259 VL) had a titer of 15 mg/L, while its counterpart IL134-0670 (identical except that it contained IL4-0002 VH instead of IL4-0157 VH) had a titer of 55 mg/L. Two other pairs of constructs also showed similar increases in titer when the VH of IL4-0157 was replaced with the VH of IL4-0002: IL134-0668 (16 mg/L rising to 66 mg/L for IL134-0671) and IL134-0669 (23 mg/L rising to 96 mg/L for IL134-0672). These observations indicated that that the IL4-0157 VH contributed heavily to low expression when anti-IL-4 was in the Fab2 position.

Expression levels were generally higher when the anti-IL-4 domain was in the Fab1 position and anti-IL-13 in the Fab2 position, compared to constructs with the same binding domains in the opposite orientation (Table 38). This was particularly evident for IL4-0157 VH/IL4-0157 VL; in this case, expression levels rose 3-12-fold when the IL4 domain was moved from Fab2 to Fab1. For example, constructs IL134-0666, IL134-0667, and IL134-0669, with anti-IL-4 in the Fab2 position, expressed at 9, 15, and 23 mg/L respectively, while their counterparts with anti-IL-4 in the Fab1 position, IL134-0731, IL134-0732, and IL134-0734 expressed at 87.5, 186.3, and 70.6 mg/L, respectively.

Both anti-IL-4 domain sequences (IL4-0157 VH/IL4-0157 VL and IL4-0002 VH/IL4-0157 VL) were compatible with high titers when the anti-IL-4 domain was in the Fab1 position. These observations suggested that the IL4-0157 VH exerted its negative effects on expression when it was located in the dual Fab LC(1) HC(2) chain of the Tri-Fab-Fc, in the Fab2 orientation, and not when expressed on the VH-CH1 modified Fd chain as it was when IL4 was in the Fab1 position.

Among anti-IL-13 domains, the IL13-0271 VL sequence emerged as a contributor to low expression. In the context of either IL-4 domain sequence analyzed in this set, the anti IL13-0271 VL paired with the IL13-0259 VH was associated with approximately two-fold lower titers than equivalent constructs in which IL13-0259 VL was paired with IL13-0259 VH (Table 38). For example, the titers of constructs with anti-IL13 in the Fab1 position increased from 9 mg/L (IL134-0666) to 15 mg/L (IL134-0667) and from 32 mg/L (IL134-0673) to 55 mg/L (IL134-0670) when IL13-0271 VL was replaced with IL13-0259 VL.

In contrast with the position-dependent expression effects of the IL4-0259 VH domain, the detrimental impact of IL13-0271 VL was similar when the anti-IL-13 domain was in either the Fab1 or the Fab2 position. Titers of constructs with anti-IL13 in the Fab2 position increased from 87.6 mg/L (IL134-0731) to 186.3 mg/L (IL134-0732) and from 52 mg/L (IL134-0738) to 90 mg/L (IL134-0735) when IL13-0271 VL was replaced with IL13-0259 VL, similar to the magnitude of effects when anti-IL13 was in the Fab1 position. An anti-IL13 domain composed of IL13-0001 VH and IL13-0001 VL was associated with generally high titers when in both Fab1 and Fab2 position.

### Example 38

### Anti-IL-4/IL-13 Dual Fab EEE Arm Engineering with CkS Designs and Production Via Transient Expi293™ Expression System

Two alternative methods of forcing specific heavy-light chain pairing in the dual Fab arm, modified Fd (mFd) and CkS (CH-CK swap), were tested for their effects on expression and purity in the context of anti-IL-4 and anti-IL-13 domains in the Fab1 and Fab2 positions as described above. The VH-CK of the Fab1 (outer) domain was fused to the VH of the Fab2 (inner Fab) heavy chain by a GGGGS (SEQ ID NO:104) linker.

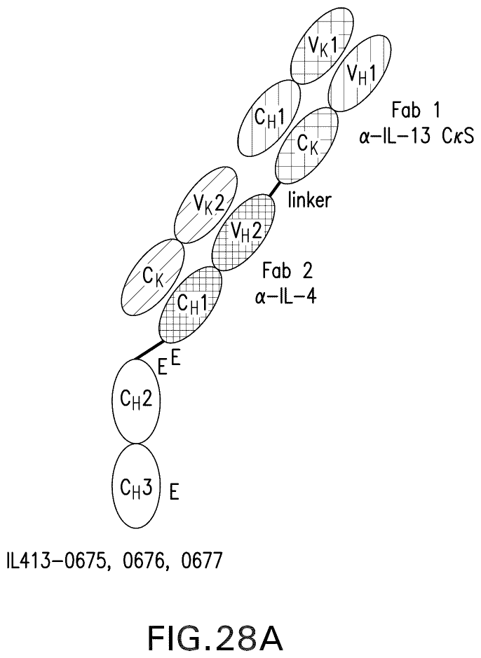

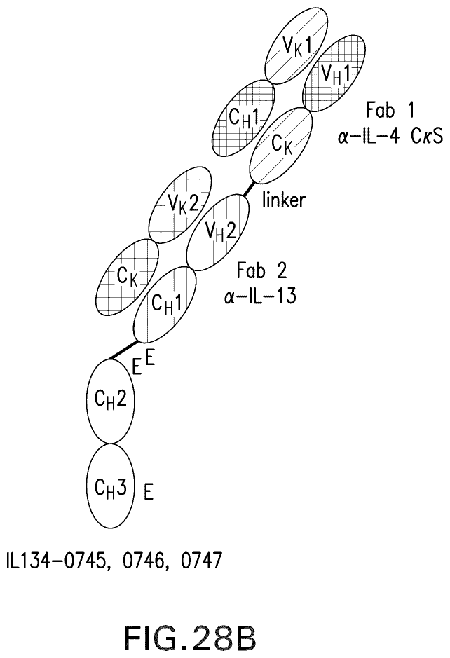

For each DFab EEE arm expression, three cDNA vectors encoding corresponding anti-IL-13/IL-4 chains (FIG. 28) were transfected into Expi 293 cells according to manufacture protocol (Thermo Fisher Scientific). The conditioned medium of cells expressing Dual Fab arms were harvested on day 5 and captured with MabSelect™ SuRe™ LX (GE Healthcare Life Sciences). The eluted antibodies were neutralized with 2M HEPES pH8.0. Non-reducing cGE was used to evaluate percentage of desired species (a mixture of “half-molecules” composed of the Dual Fab HC(1)-HC(2) chain, the VL-Ck(1) chain, and the LC(2) chain, and a homodimer of these “half molecules”) versus undesired mispaired species (non-half/homodimer). Expression titers for the resultant conditioned medium were determined with the Protein A column.

TABLE 39
Expression of dual Fab arms carrying variant IL-4 and IL-13 domains with Fab1
in CkS and mFd formats

| | | | | IL-13 Fab1 (outer); IL-4 Fab2 | | | | IL-4 Fab1 (outer); IL-13 Fab2 | | | |
|---|---|---|---|---|---|---|---|---|---|---|---|
| | | | | (inner) | | | | (inner) | | | |
| | | | | CKS | | mFd | | CkS | | | |
| | | | | Dual | | Dual | | Dual | | | |
| | | | | Fab | | Fab | | Fab | | mFd | |
| | | | | EEE | Titer | EEE | Titer | EEE | Titer | Dual Fab | Titer |
| a-IL-13 | | a-IL-4 | | Arm | (mg/L, | Arm | (mg/L, | Arm | (mg/L, | EEE Arm | (mg/L, |
| VL | VH | VL | VH | Name | proA | Name | proA | Name | proA | Name | proA |
| IL13- | IL13- | IL4- | 0157 | IL134- | 97 | IL134- | 14 | IL134- | 100 | IL134- | 87.6 |
| 0271 | 0259 | 0157 | IL4- | 0675 | | 0666 | | 0745 | | 0731 | |
| IL13- | IL13- | IL4- | 0157 | IL134- | 110 | IL134- | 23 | IL134- | 140 | IL134- | 70.6 |
| 0001 | 0001 | 0157 | IL4- | 0676 | | 0669 | | 0746 | | 0734 | |
| IL13- | IL13- | IL4- | 0002 | IL134- | 147 | IL134- | 96 | IL134- | 342 | IL134- | 88.9 |
| 0001 | 0001 | 0157 | IL4- | 0677 | | 0672 | | 0747 | | 0737 | |

When dual Fab arms were built with the Fab1 in C kappa swapped (CkS) format, expression in HEK293 was high (97-342 mg/L) for the six constructs tested; Table 39. This observation contrasted with the variable expression levels observed when the Fab1 was in mFd format, where the IL13-0271 VL domain (in Fab1) and the IL4-0157 VH (in Fab2) were associated with low expression, below 23 mg/L (dual Fab arms IL134-0666 and IL134-0667). These observations underscore the importance of the interaction between certain amino acid sequences and their position within the dual fab arm on expression; the influence of the variable regions IL13-0271 VL and IL4-0157 VH was evident only when they were on the long chain of an mFd-bearing dual Fab.

TABLE 40
Homogeneity of dual Fab arms carrying variant IL-4 and IL-13 domains with
Fab1 in CkS and mFd formats

| | | | | IL-13 Fab1 (outer); IL-4 Fab2 (inner) | | | | IL-4 Fab1 (outer); IL-13 Fab2 (inner) | | | |
|---|---|---|---|---|---|---|---|---|---|---|---|
| | | | | CKS | | mFd | | CkS | | mFd | |
| | | | | Dual | | Dual | | Dual | | Dual | |
| | | | | Fab | cGE (Non- | Fab | cGE (Non- | Fab | cGE (Non- | Fab | cGE (Non- |
| | | | | EEE | Reduced) | EEE | Reduced) | EEE | Reduced) | EEE | Reduced) |
| a-IL-13 | | a-IL-4 | | Arm | % Half/ | Arm | % Half/ | Arm | % Half/ | Arm | % Half/ |
| VL | VH | VL | VH | Name | Homodimer | Name | Homodimer | Name | Homodimer | Name | Homodimer |
| IL13- | IL13- | IL4- | IL4- | IL134- | 66.4 | IL134- | 91.8 | IL134- | 91.4 | IL134- | 77.4 |
| 0271 | 0259 | 0157 | 0157 | 0675 | | 0666 | | 0745 | | 0731 | |
| IL13- | IL13- | IL4- | 0157 | IL134- | 84.2 | IL134- | 92.8 | IL134- | 94.2 | IL134- | 79.8 |
| 0001 | 0001 | 0157 | IL4- | 0676 | | 0669 | | 0746 | | 0734 | |
| IL13- | IL13- | IL4- | \| IL4- | IL134- | 87.9 | IL134- | 94.7 | IL134- | 86.3 | IL134- | 79.3 |
| 0001 | 0001 | 0157 | 0002 | 0677 | | 0672 | | 0747 | | 0737 | |

When dual Fab arms were built with the Fab1 in C kappa swapped (CkS) format, protein homogeneity following protein A purification was generally high (desired homodimer or half molecules>84%; Table 40), with the exception of IL134-0675 (which contained binding domains IL13-0271 VL/IL13-0259 VH in the Fab1 position and IL4-0157 VL/IL4-0157 VH in the Fab2 position) at 66.4%. Low homogeneity did not follow a discernable pattern within the set of constructs examined.

Taken together, the accumulated data indicate that all of the heterodimerization methods and Fab pairing strategies that were examined are capable of supporting stable, well-expressed, and biologically-active Tri-Fab-Fc molecules, in the context of certain sequences of the three binding domains.

### Example 39

### Stable CHO Expression of Anti-IL-4/IL-13 Dual Fab EEE Arm Engineering with mFd Designs

To evaluate whether the expression and expression/pairing stability characteristics of Tri-Fab-Fc molecules produced via transient Expi293FF™ cells can also be translated into stable CHO cells, three dual Fab EEE arms in the mFd format, IL134-0666, -0667 and -0670, were examined in stable CHO cells. Three unique chains (Dual Fab LC(1)-HC(2) EEE chain, modified Fd chain and anti-IL-4 light chain) were sub-cloned into the Lonza's CHO SSI 2.0 vector. Three independent stable CHO pools (200 mL each) were generated for each Dual Fab EEE arm and expression titers for the resultant conditioned medium were determined with the Protein A column. The expression titers shown in Table 39 are the average of three pools. Capillary gel electrophoresis (cGE; LabChip, Perkin Elmer) was used to gauge the percentage of half/homodimer and non-half/homodimer.

The three dual Fab arms showed unexpected differences in expression level and purity when expressed in stably transfected CHO cells instead of transiently transfected Expi293F (Table 41). For example, although IL134-0667 and IL134-0666 were expressed at similar levels in the transient HEK293 system, in stably transfected CHO cells, IL134-0667 showed 5.5-fold improved expression titer in comparison to IL134-0666 (1337 mg/L vs. 240 mg/L). Similarly, in stably transfected CHO cells, IL134-0667 had a significantly higher proportion of the desired homodimer/half-molecule species than IL134-0666 (93.6% vs. 53.8%)

In contrast to these observations, IL134-0670 had 3.6-fold higher expression than IL134-667 in transient Expi293F™, but 2-fold lower expression in stably transfected CHO cells.

From this limited set of molecules, the lowest expression in stable CHO cells was correlated with the VL of IL13-0271 in the outer position of the Dual Fab LC(1)-HC(2) chin, while the presence of the VH of IL4-0002 in the inner position of the Dual Fab LC(1)-HC(2) chain did not correlate with low expression. This stands in contrast with the pattern observed in transient Expi293F™ expression, where the VH of IL4-0002 in this position was associated with the poorest expression. While in both expression systems, small numbers of amino acid changes led to large differences in expression and purity, the impact of specific sequences was not translatable from one system to the other.

TABLE 41
Expression level and purity of mFd EEE DFab arm designs in stable CHO and transient Expi293F ™ cells

| | | | | | Transient | | | |
|---|---|---|---|---|---|---|---|---|
| | | | | | Expi293F ™ cell | | Stable CHO cells | |
| Dual Fab | Fab 1 | | Fab 2 | | | cGE (Non- | | cGE (Non- |
| EEE | anti-IL-13 | | anti-IL-4 | | Expression | Reduced) | Express | Reduced) |
| Arm | (outer) | | (inner) | | Titer | % Half/ | ion Titer | % Half/ |
| Name | VL | VH | VL | VH | (mg/L) | Homodimer | (mg/L) | Homodimer |
| IL134- | IL13- | IL13- | IL4- | IL4- | 14 | 91.8 | 240 | 53.8 |
| 0666 | 0271 | 0259 | 0157 | 0157 | | | | |
| IL134- | IL13- | IL13- | IL4- | IL4- | 15 | 89.4 | 1337 | 96.3 |
| 0667 | 0259 | 0259 | 0157 | 0157 | | | | |
| IL134- | IL13- | IL13- | IL4- | IL4- | 55 | 82.3 | 655 | 88.9 |
| 0670 | 0259 | 0259 | 0157 | 0002 | | | | |

### Example 40

### Simultaneous Binding of 3 Cytokines by Tri-Fab-Fc Variants

Surface plasmon resonance (SPR) was used to determine if the Tri-Fab-Fc could simultaneously bind three cytokines. For this analysis, a semi-quantitative SPR method was developed using BIAcore 8K+ (GE Healthcare) instrumentation. For this evaluation, an anti-human IgG antibody (GE Healthcare, BR-1008-39) was covalently amine coupled onto all flow cells of a CM5 carboxymethylated dextran coated sensor chip to a density of about 10,000 resonance units (RU) following the manufacturer's protocol and then IL13433-0006 Tri-Fab-Fc was captured to a level of approximately 60-90 RUs. Next, in the first cycle the Tri-Fab-Fc was saturated with one of the three cytokines, followed by saturation with the other cytokines in subsequent cycles or buffer only in cycles 1 and 2 (Table 42).

TABLE 42

| Alternating Cytokines | | | | Buffer only followed by cytokine | | | |
|---|---|---|---|---|---|---|---|
| Cycle | 5 | 7 | 9 | Cycle | 11 | 13 | 15 |
| 1^{st} injection | IL-13 | IL-33 | IL-4 | 1^{st} injection | Buffer | Buffer | IL4 |
| 2^{nd} injection | IL-33 | IL-4 | IL-13 | 2^{nd} injection | Buffer | Buffer | IL13 |
| 3^{rd} injection | IL-4 | IL-13 | IL-33 | 3^{rd} injection | Buffer | Buffer | IL-33 |

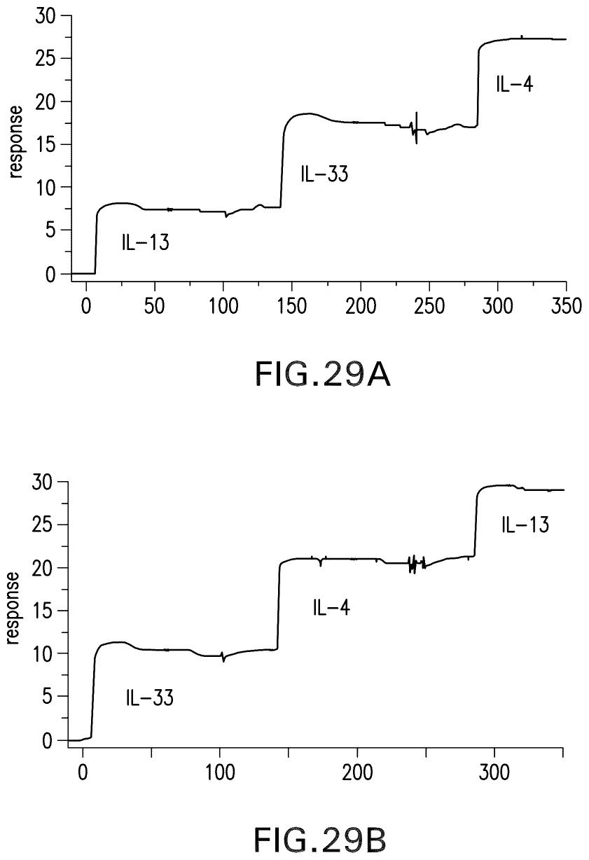

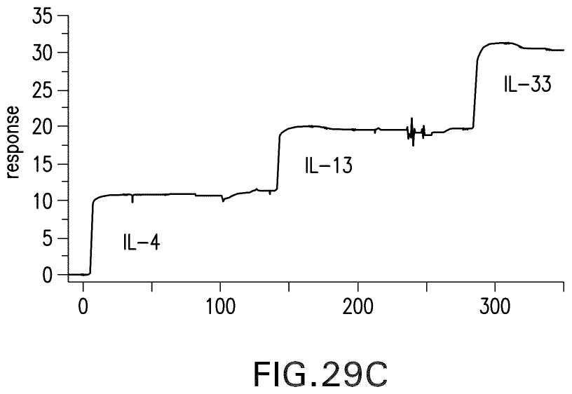

Results from this SPR evaluation indicate that the IL13433-0006 Tri-Fab-Fc can simultaneously bind the IL-4, IL-13 and IL-33 cytokines (FIG. 29). Additionally, the 3^{rd} injection percent activity results are similar for buffer-buffer-cytokine and cytokine1-cytokine2-cytokine3 cycles further supporting that when the Tri-Fab-Fc molecule is bound to one or two cytokines, it can bind as much of the third cytokine as it could in the absence of the other two cytokines.

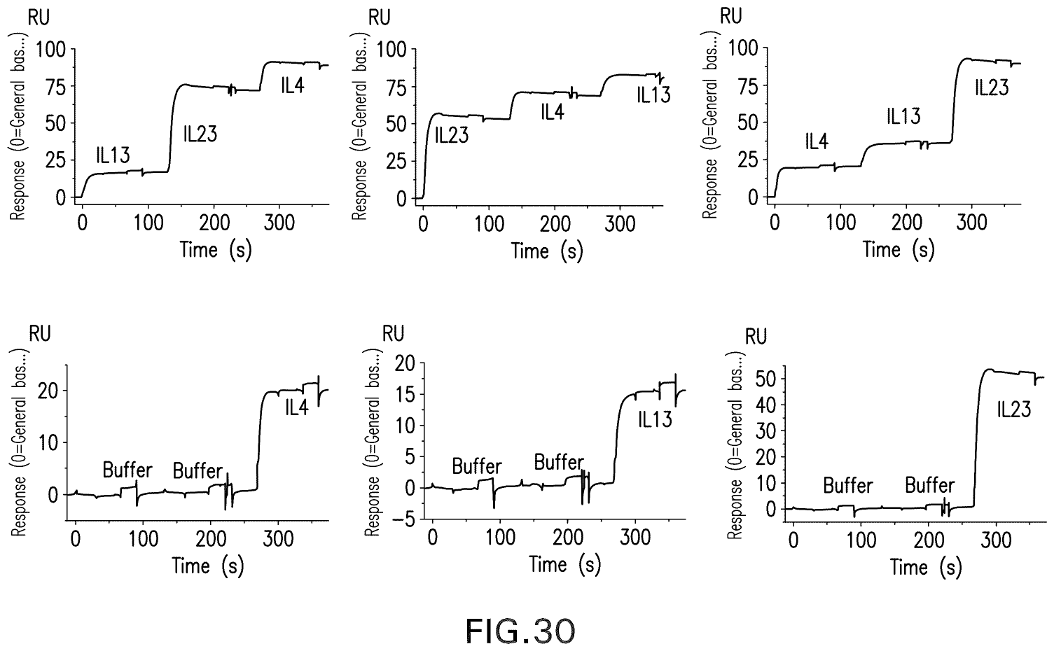

Similarly, IL413p40-0705 was tested for simultaneous binding to IL-4, IL-13, and IL-23 (with 40 subunit) using the methods described above, except that approximately 100 resonance units of Tri-Fab-Fc were captured on the sensor surface. IL413p40-0705 simultaneously binds to all three cognate cytokines, IL-13, IL-4 and IL-23 (with p40 subunit), regardless of order of injection (FIG. 30).

### Example 41

### Optimization of Charge-Based Fc Heterodimerization Method for Improvement of Pharmacokinetic Properties

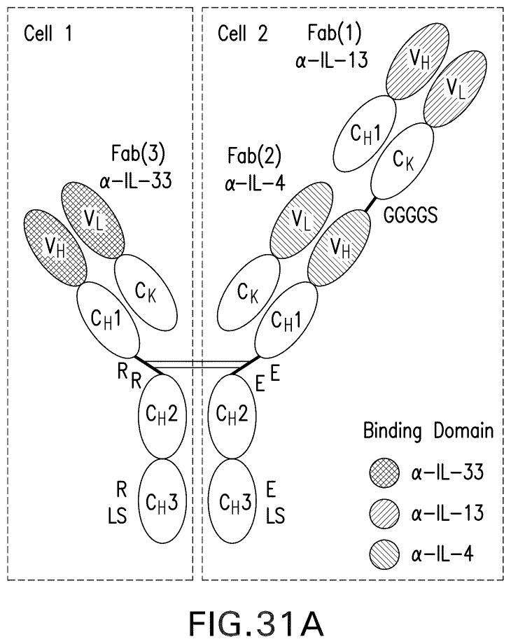

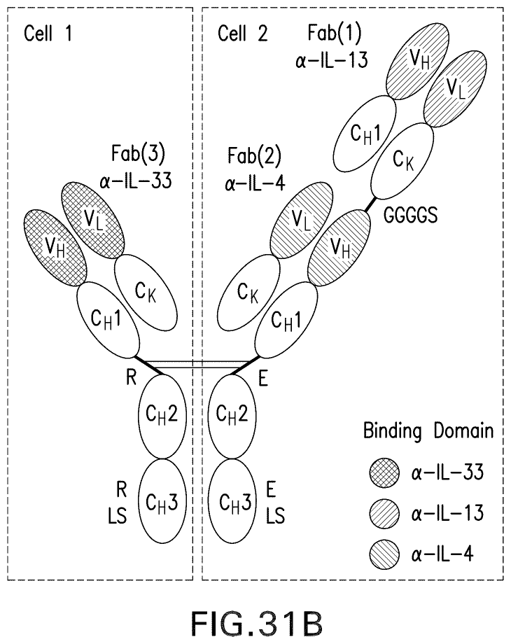

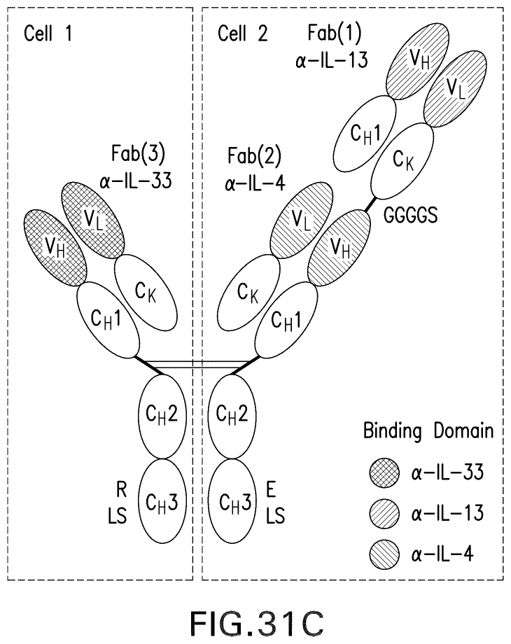

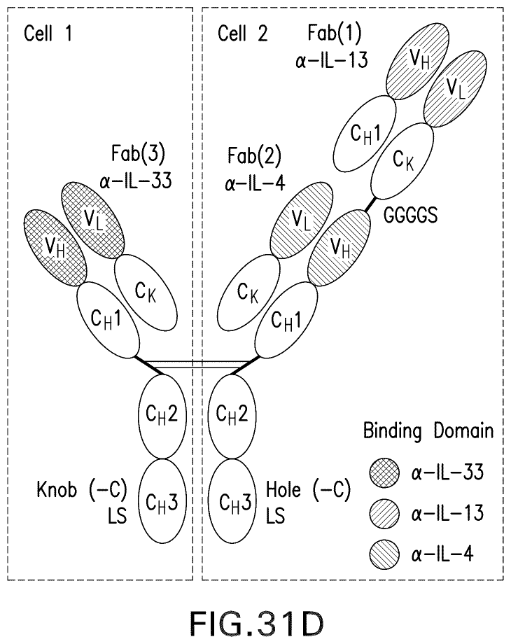

A series of anti-IL-4/IL-13/IL-33 (IL13433) Tri-Fab-Fc variants was designed for expression using the dual cell approach, in which the dual Fab arm and single Fab arm are expressed in separate cells. Variants were primarily engineered in which Fc heterodimerization is mediated by opposite charge-paired mutations (FIGS. 31A, 31B and 31C), but Knob-into-Hole mutations were also used to engineer a Tri-Fab-Fc variant, IL13433-1275 (FIG. 31D). All dual cell approach designs utilized the same positioning of the Fab domains as in IL13433-1270 (FIG. 23D). Specifically, one cell produced the single Fab arm with the anti-IL-33 binding domain IL33-0726 in the Fab3 position, and the second cell produced the dual Fab arm, composed of the anti-IL-13 binding domain IL13-0001 in mFd format in the Fab1 or outer position and an anti-IL-4 domain in native Fab format in the Fab2 or inner position (FIG. 31). The anti-IL-4 domain IL4-1040 was used in Tri-Fab-Fc molecules IL13433-1258, IL13433-1261, IL13433-1270, and IL13433-1275, while the anti-IL4-domain IL4-0749 was used in IL13433-1042. In one instance, three positively charged residues were introduced into the human IgG1 constant region to drive Fc heterodimerization: two mutations D(H232)R and P(H241)R into the hinge and K(H440)R into the CH3 region. The three opposite negative charge-paired mutations incorporated into human IgG1 constant region were D(H232)E and P(H241)E into the hinge region and L(H391)E into the CH3 domain. This design that utilizes three charge-paired mutations, referred to as “EEE/RRR,” was used to engineer the Tri-Fab-Fc variant IL13433-1042 (FIG. 31A). Variations of opposite charge-paired mutations were used to construct Tri-Fab-Fc molecules where the amount of excess charge was reduced in the antibody hinge region. Tri-Fab-Fc IL13433-1258 was engineered to include only D(H232)R or D(H232)E in the hinge region and L(H391)E or K(H440)R only in the CH3 domain (“EE/RR;” FIG. 31B), while IL13433-1261 contains no charged mutations in the hinge region and harbors L(H391)E or K(H440)R only in the CH3 domain (“E/R;” FIG. 31C).

### Example 42

### Purification of Tri-Fab-Fc Variant Proteins Produced by Single Cell and Dual Cell Methods for Pharmacokinetic Evaluation

Anti-IL-4/IL-13/IL-33 Tri-Fab-Fc variants were generated using both single cell and dual cell approaches and conditioned medium containing these Tri-Fab-Fc proteins were produced using Expi293F™ host cells with the manufacturer's recommended protocol. The general method for purifying single cell derived Tri-Fab-Fc variants uses a three-column step: MabSelect™ SuRe™ LX (GE Life Sciences), followed by Mono-S Cation Exchange, Superdex 200 gel filtration and then buffer exchange into PBS-CMF pH 7.4 (Table 43). In some instance for the single cell produced Tri-Fab-Fc, the Superdex 200 gel filtration step can be eliminated after process development optimization. The general purification process for dual cell produced Tri-Fab-Fc variants was the following, with molecule-specific details noted in Table 43. MabSelect™ SuRe™ LX capture of the Dual Fab homodimer and the Single Fab homodimer, redox reaction that varies with choice of opposite charge-paired mutations in Fc region, followed by buffer exchange, Mono-S cation exchange and final buffer exchange into PBS-CMF pH 7.4. Further, for some dual cell produced Tri-Fab-Fc, Superdex 200 gel filtration chromatography was included after the cation exchange step. These results support that methods can be developed to purify Tri-Fab-Fc variants produced via the single cell or dual cell approach, which each have their own benefits and challenges. For the dual cell approach, redox conditions vary depending on which Fc heterodimerization mutations are utilized, and extensive screening for appropriate conditions is required. For the single cell approach, although the expression can be challenging since the 5 chains comprising the Tri-Fab-Fc need to be produced in a single cell, the overall purification method is closer to a standard antibody process and does not require multiple purification steps to obtain the molecule of interest. The single cell approach has increased complexity from an analytical perspective, since production of components from each dual cell, specifically dual Fab(1)-Fab(2) homodimer and single Fab(3) homodimer, would be required to analyze mispaired species.

TABLE 43
Purification Scheme for Single Cell and Dual Cell Expi293F ™ Produced
Tri-Fab-Fc Variants.

| Tri-Fab- | | | | | |
|---|---|---|---|---|---|
| Fc | | | | Post-redox | |
| Variant | Approach | ProA capture | Redox | purification | Analytical |
| IL13433- | Dual Cell: | +Platform | +20x Cys | +2 column | +LC pairs |
| 1042 | EEE/RRR | | at 25° C. | purification | correctly when |
| | | | | steps (CEX, | expressing in |
| | | | | SEC) | dual cell format |
| | | | | | +Parental |
| | | | | | homodimer |
| | | | | | detection with |
| | | | | | aCEX (Agilent |
| | | | | | 1290) |
| IL13433- | Dual Cell: | +Platform | −high | +good | +LC pairs |
| 1258 | EE/RR | | reductant | separation on | correctly when |
| | | | molar | CEX (2-step | expressing in |
| | | | excess | purification) | dual cell format |
| | | | (300x GSH) | | +Parental |
| | | | | | homodimer |
| | | | | | detection with |
| | | | | | aCEX (Agilent |
| | | | | | 1290) |
| IL13433- | Dual Cell: | +Platform | −high Cys | +good | +LC pairs |
| 1261 | E/R | | molar | separation on | correctly when |
| | | | excess | CEX (2-step | expressing in |
| | | | (150x) | purification) | dual cell format |
| | | | −overnight | | +Parental |
| | | | reaction at | | homodimer |
| | | | 37° C. | | detection with |
| | | | | | aCEX (Agilent |
| | | | | | 1290) |
| IL13433- | Dual Cell: KiH, | −tris/arginine | −high GSH | +good | +LC pairs |
| 1275 | no engineered | to stabilize | molar | separation on | correctly when |
| | Cys in CH3 | Kik and HiH | excess | CEX (2 step | expressing in |
| | domain | dimers | (600x) | purification) | dual cell format |
| | | | −overnight | | +Parental |
| | | | reaction at | | detection with |
| | | | 37° C. | | aCEX (Agilent |
| | | | | | 1290) |
| IL13433- | Single Cell: | +Platform | +No redox | +good | −limited |
| 1270 | KIH, S1-S1 | | needed | separation on | analytical |
| | reverse | | | CEX (2-step | methods to |
| | | | | purification) | detect LC |
| | | | | | mispairing other |
| | | | | | than LCMS |
| IL13433- | Single Cell: | +Platform | +No redox | +good | −limited |
| 1279 | KiH, S1-S1 | | needed | separation on | analytical |
| | reverse, no | | | CEX (2-step | methods to |
| | engineered | | | purification) | detect LC |
| | Cys in CH3 | | | | mispairing other |
| | domain | | | | than LCMS |

+= favorable property; −= negative property; aCEX = analytical Cation Exchange; CEX = Mono-S Cation Exchange; Cys = Cysteine; GSH = Glutathione; HiH = Hole-into-Hole; KiH = Knob-into-Hole; KiK = Knob-into-Knob; Platform = according to manufacturer's recommended protocol.

### Example 43

### Bioanalytical and Biophysical Characterization of Tri-Fab-Fc Variants

Tri-Fab-Fc variants produced using either the single cell or dual cell approaches were subjected to extensive bioanalytical and biophysical characterization to understand the properties of these complex molecules prepared using different Fc heterodimerization and charge paired mutations to limit mispairing of the protein chains and encourage maximum yield of intact Tri-Fab-Fc molecule. In particular, the following methods were used to assess key molecular properties: analytical size-exclusion chromatography (aSEC) to determine percent high molecular mass species (HMMS) as an indicator of aggregation, thermal stability using Differential Scanning Calorimetry (DSC), non-reduced cGE to assess percent peak of interest (POI), imaged capillary electrophoresis (iCE) for unstressed sample (TO) to evaluate charge heterogeneity, in silico immunogenicity assessment score and non-specificity evaluation. Details for each method are the following.

Analytical SEC was performed using a YMC-Pack Diol-200 SEC column in 20 mM Na₃PO₄, 400 mM NaCl, pH 7.2 buffer. Retention time and peak width of the main peak as well as the areas and percent areas of the main (POI), low molecular mass species (LMMS) and HMMS peaks were recorded and used to calculate percent main (POI), HMMS and LMMS. For the DSC method, samples at 0.3 mg/mL were dispensed into the sample tray of a MicroCal VP-Capillary DSC with Autosampler (Malvern Instruments, Inc.), equilibrated for 5 minutes at 10° C. and then scanned up to 110° C. at a rate of 100° C. per/hour. A filtering period of 16 seconds was selected. Raw data was baseline corrected and the protein concentration was normalized. Origin Software 7.0 (Origin Lab Corporation, Northampton, MA) was used to fit the data to an MN2-State Model with an appropriate number of transitions.

Non-reduced cGE was performed using the Caliper LabChip GXII (PerkinElmer Inc., Hopkinton, MA) according to manufacturer's recommended protocol.

Protein Simple iCE3 instrument with PrinCE Autosampler (ProteinSimple, San Jose, CA) was used to analyze charge heterogeneity for the unstressed (TO) Tri-Fab-Fc samples. Proteins were diluted to 2 mg/mL in water. Sample diluent is comprised of 0.01 mg/mL pI marker 4.65, 0.01 mg/mL pI marker 9.5, 4.0% Pharmalyte pH 3-10, 0.25% methyl cellulose, and 2.0 M urea. Samples contained 15 μL protein at 2 mg/mL and 85 μL sample diluent. Samples were focused for 1 minute at 1500 Volts and then 6 minutes at 3000 Volts.

To calculate immunogenicity scores, sequences are submitted for EpiMatrix analysis in the ISPRI software package (ISPRI v 1.8.0, EpiVax Inc., Providence, RI; 26). The raw results provide rankings of likelihood of binding of each 9-mer amino acid fragment against 8 different HLA types. Analysis of clinical anti-drug-antibody (ADA) data and known risk factors such as target location or biophysical properties led to the following guidance for using the T-reg Adjusted Pfizer Score. Ranking of scores are the following: Good ≤−50, Moderate ≤−30 and ≥−50, Poor ≥−30.

The AC-SINS assay and DNA and insulin direct binding ELISA methods are described in EXAMPLE 15. Ranking of scores is the following: Good 0-5, Moderate >5 and <10, High >10. The summary of results for the biophysical and bioanalytical evaluation of anti-IL-4/IL-13/IL-33, IL-4/IL-13/TSLP, and IL-4/IL-13/p40 Tri-Fab-Fc variants generated using either the single cell or dual cell approach and engineered with alternate Fc heterodimerization mutations indicate that all generally have favorable molecular properties comparable to standard well-behaved antibodies (Table 44). All exhibit good thermal stability with Tₘ1 values >65° C. Integrity of intact Tri-Fab-Fc is >95% as determined with non-reducing cGE assay and propensity to aggregate was deemed low by analytical SEC analysis. These Tri-Fab-Fc variants present good in silico predicted immunogenicity and acceptable non-specificity scores that are a composite of binding domains comprising the Tri-Fab-Fc that were engineered for these favorable characteristics. One observation noted for the Tri-Fab-Fc variants, unlike that for well-behaved antibodies, is increased charge heterogeneity that may be due to complexity of a single molecule harboring three independent Fab binding domains.

TABLE 44
Summary of Key Biophysical and Bioanalytical Properties Evaluated for Tri-Fab-Fc
Variants.

| | | | | | | | | | Non-specificity | | |
|---|---|---|---|---|---|---|---|---|---|---|---|
| | | | | | | | | | Scores | | |
| | | | | | | | | Immuno- | (n = 2 | | |
| | | | | | | | | genicity | independent | | |
| | | | | | | | | Pfizer | assays if | | |
| | | | | cGE | | | | T-reg | multiple values | | |
| Tri- | | | DSC | % | iCE T0 | | | Adjusted | reported) | | |
| Fab-Fc | | aSEC % | Tₘ1 | Intact | % | % | % | Score | AC- | | |
| Variant | Description | HMMS | (° C.) | NR | acidic | main | basic | (Multichain) | SINS | DNA | Insulin |
| IL1343 | Dual | 0.7 | 69.84 | ND | 37.6 | 550 | 7.4 | −53.70 | 12 | 3 | 3 |
| 3-1042 | Cell: | | | | | | | | | | |
| | EEE/R | | | | | | | | | | |
| | RR | | | | | | | | | | |
| IL1343 | Dual | 1.33 | 70.47 | 98.1 | 24.5 | 66.4 | 9.2 | −53.32 | 1. 6 | 6.5 | 6.5 |
| 3-1258 | Cell: | | | | | | | | | | |
| | EE/RR | | | | | | | | | | |
| IL1343 | Dual | 1.84 | 70.30 | 96.7 | 21.8 | 69.8 | 8.4 | −53.32 | 1. 5 | 5.6 | 6.6 |
| 3-1261 | Cell: | | | | | | | | | | |
| | E/R | | | | | | | | | | |
| IL1343 | Dual | 0.74 | 70.10 | 95.8 | 48.1 | 46.1 | 5.9 | −53.32 | 1. 4 | 6.5 | 6.6 |
| 3-1275 | Cell: | | | | | | | | | | |
| | KiH, No | | | | | | | | | | |
| | Cys in | | | | | | | | | | |
| | CH3 | | | | | | | | | | |
| IL1343 | Single | 0.26 | 70.92 | 97.1 | 25.2 | 65.8 | 9.0 | −53.32 | 1. 5 | 7.5 | 7.6 |
| 3-1270 | Cell: | | | | | | | | | | |
| | KiH, | | | | | | | | | | |
| | S1- | | | | | | | | | | |
| | S1rev | | | | | | | | | | |
| IL1343 | Single | 0.5 | 71.52 | ND | 41.6 | 49.5 | 8.9 | −49.74 | 1 | 3 | 2 |
| 3-0006 | Cell: | | | | | | | | | | |
| | KiH, | | | | | | | | | | |
| | S1- | | | | | | | | | | |
| | S1rev | | | | | | | | | | |
| IL1343 | Single | 0.09 | 71.60 | ND | 26.6 | 61.5 | 11.9 | −60.40 | 3 | 4.5 | 6.7 |
| 3-0607 | Cell: | | | | | | | | | | |
| | KiH, | | | | | | | | | | |
| | S1- | | | | | | | | | | |
| | S1rev | | | | | | | | | | |
| IL413T | Dual | 0.18 | 69.01 | 99.0 | 27.2 | 65.3 | 7.5 | −57.01 | 9 | 4 | 6 |
| SLP- | Cell: | | | | | | | | | | |
| 1024 | EE/RR | | | | | | | | | | |
| IL413T | Dual | 0.45 | 70.48 | 91.6 | 23.7 | 72.2 | 4.1 | −56.94 | 4.2 | 2.3 | 9. 6 |
| SLP- | Cell: | | | | | | | | | | |
| 1028 | EE/RR | | | | | | | | | | |
| IL413T | Dual | 0.71 | 68.82 | 91.4 | 25.0 | 70.6 | 4.4 | −57.01 | 4.2 | 1.3 | 9. 6 |
| SLP- | Cell: | | | | | | | | | | |
| 1037 | EE/RR | | | | | | | | | | |
| IL413p | Dual | 0.98 | 69.47 | 99.5 | 27.3 | 56.3 | 16.3 | −30.83 | 3.2 | 5.4 | 8.7 |
| 40- | Cell: | | | | | | | | | | |
| 0705 | EE/RR | | | | | | | | | | |

For non-specificity scores, multiple scores represent n = 2 independent assays; ranking of immunogenicity scores are the following: Good ≤−50, Moderate ≤−30 and ≥−50, Poor ≥−30; scores is the following: Good 0-5, Moderate >5 and <10, High >10; Ranking of AC-SINS and polyreactivity scores are the following: Good 0-5, Moderate >5 and <10, High >10; KiH = Knob-into-Hole; ND = not determined; S1-S1rev = electrostatic complementary S1 and S1-reverse mutations.

Intact liquid chromatography-mass spectrometry (LCMS) analysis was performed on select Tri-Fab-Fc variants to determine the amount of desired intact correctly paired Tri-Fab-Fc molecule present within the final purified product. Tri-Fab-Fc samples were incubated with PNGaseF (New England Biolabs, Npswich MA) for 1 hour at 37DC. Next, 25 μg of sample was injected on a C4 BEH300 column (Waters™) and analyzed by LCMS analysis on a Waters Acquity H-Class HPLC coupled with a Bruker maXis II QTOF mass spectrometer. Results from the intact LCMS analysis show that the desired intact Tri-Fab-Fc is the major molecular entity (>95%) with only trace amounts (<5%) of undesired biproducts in the final purified product for these variants produced using either the single cell or dual cell approach and engineered with alternate Fc heterodimerization mutations (Tables 45 and 46).

TABLE 45
Results of Intact LCMS Analysis for Single
Cell Produced Tri-Fab-Fc Variants

| | | Undesired Biproducts | |
|---|---|---|---|
| Tri-Fab-Fc Variant | Intact | Tri-Fab-Fc | Tri-Fab-Fc |
| ID | Tri-Fab-Fc | minus SFab LC | minus Dfab LC |
| IL13433-1270 | Major | Trace | ND |
| IL13433-1279 | Major | Trace | Trace |

ND = not detected; amount detected: Trace (<5%), Minor (<50%) and Major (>50%)

TABLE 46
Results of Intact LCMS Analysis for Dual Cell Produced
Tri-Fab-Fc Variants

| | | Undesired Biproducts | | | | |
|---|---|---|---|---|---|---|
| | | EE/E/Hole | Tri- | Tri- | Tri- | EE/E/Hole |
| | Intact | Dual Fab | Fab- | Fab- | Fab- | Dual Fab |
| Tri-Fab-Fc | Tri- | Arm | Fc | Fc | Fc | Arm |
| Variant | Fab- | Homo- | minus | minus | minus | Mono- |
| ID | Fc | dimer | SFab LC | DFab LC | mFd | mer |
| IL13433- | Major | Trace | Trace | Trace | Trace | Trace |
| 1258 | | | | | | |
| IL13433- | Major | Trace | Trace | Trace | Trace | Trace |
| 1261 | | | | | | |
| IL13433- | Major | ND | Trace | Trace | Trace | Trace |
| 1275 | | | | | | |
| IL413TSLP- | Major | ND | Trace | Trace | Trace | ND |
| 1024 | | | | | | |
| IL413TSLP- | Major | ND | Trace | Trace | Trace | ND |
| 1028 | | | | | | |
| IL413TSLP- | Major | ND | Trace | Trace | Trace | ND |
| 1037 | | | | | | |
| IL413p40- | Major | ND | Trace | Trace | Trace | Trace |
| 0705 | | | | | | |

ND = not detected; amount detected: Trace (<5%), Minor (<50%) and Major (>50%)

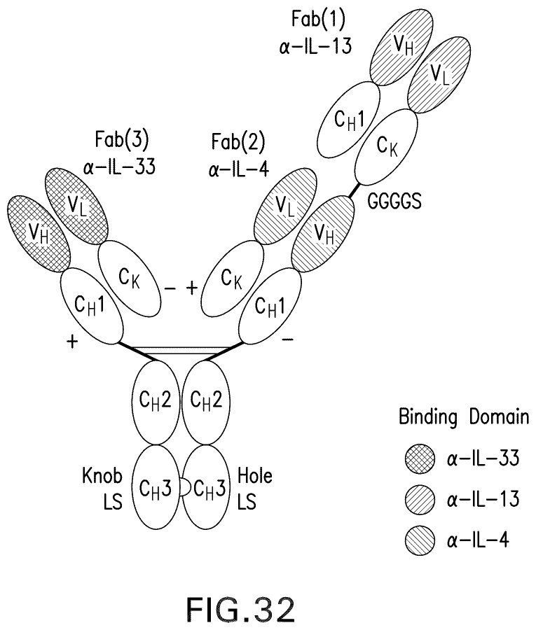

A comprehensive molecular assessment was performed for IL13433-1258, IL413TSLP-1024, IL413TSLP-1028, IL413TSLP-1037 and IL313p40-0705 Tri-Fab-Fc variants produced using the dual cell process (FIG. 31) composed of the following binding domains in a fixed geometry: IL13-0001 (Fab1), IL4-1040 (Fab2), and either IL33-0726, TSLP-0875, TSLP-0855, TSLP-0871 or p40-0003 (Fab3). Similarly, a comprehensive molecular assessment was also performed for IL13433-1270, the anti-IL-4/IL-13/IL-33 Tri-Fab-Fc produced using the single cell (FIG. 32) process, with identical binding domain geometry as IL13433-1258. The aim of this further analysis was to understand impact of stressing the Tri-Fab-Fc variants subjected to thermal and high concentration challenges on molecular integrity and potential induction of chemical liabilities. For the high concentration solubility and stability assessment, the Tri-Fab-Fc variants were formulated to 150 mg/mL into three formulation buffers (20 mM Tris, 8.5% sucrose pH 7.5, 20 mM Histidine, 8.5% sucrose, 0.05 mg/mL EDTA pH 5.8 and 20 mM Glutamic acid, 8.5% Trehalose pH 4.5) and analysis was done at 25° C. For the stressed sample analysis to evaluate chemical modifications to the inherent protein sequence, the Tri-Fab-Fc variants were formulated to 5 mg/mL into three formulation buffers minus excipients (20 mM Tris pH 7.5, 20 mM Histidine pH 5.8 and 20 mM Glutamic acid pH 4.5), incubated at 40° C. and samples removed at 0, 2, and 4 weeks for evaluation.

Results from the comprehensive molecular analysis support findings from the initial biophysical and bioanalytical assessment (Table 44) that all of the IL13433-1258, IL13433-1270, IL413TSLP-1024, and IL413p40-0705 Tri-Fab-Fc variants have overall acceptable to good properties consistent with that of a well-behaved antibody in a standard IgG format (Table 47). Across all IL13433-1258, IL13433-1270, IL413TSLP-1024 and IL413p40-0705 Tri-Fab-Fc variants, at high concentration (˜150 mg/mL) in the platform His-Sucrose formulation buffer and the charge profile is stable as determined by iCE analysis. Across all IL13433-1258, IL13433-1270, IL413TSLP-1024 and TL413p40-0705 Tri-Fab-Fc variants, there is minimal increase in fragmentation for samples stored at 25° C. for 6 weeks by cGE. As expected, the level of fragmentation is higher for samples stored at 40° C. for 4 weeks, but still minimal in the His pH 5.8 buffer with no excipients. Importantly, across most variants, the charge profile (iCE) is stable with minimal to acceptable increase in basic or acidic species at 25° C. and after 40° C. forced degradation, except IL413p40-0705 has moderate increase in basic species at 250 that was not observed in other Tri-Fab-Fc variants. Across all L13433-1258, L13433-1270, IL413TSLP-1024 and IL413p40-0705 Tri-Fab-Fc variants, no loss of bioactivity was observed at 25° C. and after 40° C. forced degradation, supporting that sequence liabilities were removed by engineering from the IL-4 CDRs in all the Tri-Fab-Fc variants and IL-33 CDRs in anti-IL-4/IL-13/IL-33 Tri-Fab-Fc. These results also show that both the dual cell and the single cell approach processes can be successfully used to produce Tri-Fab-Fc molecules with good solubility and stability consistent with a standard monoclonal antibody.

TABLE 47
Comprehensive Molecular Assessment for IL-4/IL-13/IL-33, IL-4/IL-13/TSLP and IL-4/IL-
13/p40 Tri-Fab-Fc Variants Engineered Using the Single Cell or Dual Cell Modality.

| | | IL13433- | IL413TSLP- | IL1413p40- |
|---|---|---|---|---|
| | IL13433-1258 | 1270 Tri- | 1024 Tri-Fab- | 0705 Tri-Fab- |
| Property | Tri-Fab-Fc | Fab-Fc | Fc | Fc |
| Stability upon pH 3.4 hold | EE and RR | Stable at | EE and RR | EE and RR |
| | homodimers | pH 4.0; | homodimers | homodimers |
| | stable | minor | stable | stable |
| | | instability | | |
| Solubility at ≥150 mg/mL | Achieved | Achieved | protein loss at | Achieved |
| | | | high conc > | |
| | | | 130 mg/mL in | |
| | | | His/Suc | |
| Stability at ≥150 mg/mL | Minimal | Minimal | Minimal | Minimal |
| (rate of increase in HMMS | | | | |
| and/or LMMS up to 6 weeks | | | | |
| at 25° C. | | | | |
| 25° C. Stability (ICE) (rate of | Minimal | Minimal | Minimal | Moderate |
| rate increase in acidic | | | | increase in |
| and/or basic species up to 6 | | | | acidic species, |
| weeks at 25° C.) | | | | 6 weeks at |
| | | | | 25° C. |
| 25° C. Stability (cGE) (Rate | No significant | No | No significant | No significant |
| of rate increase in | increase | significant | increase | increase |
| fragmentation up to 6 weeks | | increase | | |
| at 25° C.) | | | | |
| 40° C. Stability (aSEC) (rate | Minimal | Minimal | Minimal | Acceptable |
| of rate increase in HMMS | | | | |
| and/or LMMS up to 6 weeks | | | | |
| at 40° C.) | | | | |
| 40° C. Stability (aSEC-MALS) | Minimal | Not Done | None | Not Done |
| (level of oligomeric | | | detected | |
| aggregates detected up to 4 | | | | |
| weeks at 40° C.) | | | | |
| 40° C. Stability (ICE) (rate of | Acceptable | Acceptable | Minimal in His | Acceptable |
| rate increase for acidic | | | buffer | |
| and/or basic species up to 4 | | | | |
| weeks at 40° C.) | | | | |
| 40° C. Stability (cGE) | Minimal | Minimal | Minimal | Minimal |
| (fragmentation observed up | | | | |
| to 4 weeks at 40° C.) | | | | |
| 40° C. Stability (Bioassay) | None detected | None | None | None detected |
| (loss in bioactivity) | | detected | detected | |

HMMS = high molecular mass species: LMMS = low molecular mass species.

### Example 44

### Incorporation of E(L93)K into CDRL3 of the IL-4 Binding Domain Reduces Viscosity of IL13433-1258 and IL413p40-0700 Tri-Fab-Fc Variants

The single amino acid substitution E(L93)K (Pfabat numbering) was shown to reduce viscosity of the anti-IL-4 antibody variant IL4-0002 (EXAMPLE 7), so this mutation was engineered into the anti-IL-4/13/33 Tri-Fab-Fc format to understand if it would also reduce viscosity in the environment of this multispecific molecule. The IL13433-1042 Tri-Fab-Fc variant produced with the dual cell approach via opposite charge mutations (FIG. 31A) is composed of the following binding domains engineered into the specified geometry: IL13-0001 (Fab1), IL4-0749 (Fab2) and IL33-0726 (Fab3). Introduction of E(L93)K into the IL4-0749 V_{L} CDRL3 resulted in IL4-1040 anti-IL-4 antibody variant (V_{H} SEQ ID NO. 30 and V_{L} SEQ ID NO. 34). The IL4-1040 Fab harboring E(L93)K along with the IL13-0001 and IL33-0726 Fabs were engineered into the Tri-Fab-Fc modality using both an opposite charge based dual cell (FIG. 31B) and single cell KiH (FIG. 31D) design, thus generating IL13433-1258 and IL13433-1270, respectively. The binding domain (Fab) geometry for IL13433-1258 and IL13433-1270 is identical to that of IL13433-1042. Viscosity was assessed for the Tri-Fab-Fc variants using the DLS method. Precisely, proteins buffer exchanged into 20 mM Histidine, 8.5% sucrose, 0.05 mg/mL EDTA pH 6.0 were concentrated using Vivaspin centrifugal concentrators 10K MWCO (GE Healthcare) and aliquots were removed from the concentrator retentate as the protein concentration increased. Next, 300 nm beads (Nanosphere, Thermo Scientific) diluted 1:10 into 20 mM histidine, 8.5% sucrose, 0.05 mg/mL EDTA pH 6.0 buffer were added to the protein samples and buffer blank. The protein/bead samples were mixed by gently and sample was transferred to 1536 well plate (SensoPlate, glass bottom, Greiner Bio-One) for analysis by dynamic light scattering (DLS) using a DynaPro Plate Reader (Wyatt Technology, Santa Barbara, CA). Results from the DLS viscosity assessment confirm that the E(L93)K single amino acid substitution within the IL-4 binding domain also reduces viscosity of the IL13433-1258 and IL13433-1270 Tri-Fab-Fc variants and that this reduction is more pronounced at higher concentrations (Table 48). Further, these results show that viscosity profiles are comparable for Tri-Fab-Fc variants composed of identical binding domains, but produced using either the dual cell (IL13433-1258, FIG. 31B) or the single cell approach (IL13433-1270, FIG. 31D) and indicates modality or method of preparation do not impact viscosity (Table 48).

TABLE 48
The E(L93)K single amino acid substitution within the IL-4 binding domain reduces the
viscosity of the IL13433-1258 Tri-Fab-Fc as demonstrated using the DLS method.

| IL13433-1042 | | | IL13433-1258 (+E93K) | | | IL13433-1270 (+E93K) | | |
|---|---|---|---|---|---|---|---|---|
| Concentration | Viscosity | STD | Concentration | Viscosity | STD | Concentration | Viscosity | STD |
| [mg/mL] | cP | DEV | [mg/mL] | cP | DEV | [mg/mL] | cP | DEV |
| 23.5 | 8.2 | 2.1 | 23.5 | 4.3 | 0.9 | 23.5 | 6.0 | 1.3 |
| 47.1 | 11.8 | 2.2 | 70.6 | 8.6 | 1.4 | 47.1 | 6.7 | 1.3 |
| 94.1 | 22.6 | 4.7 | 94.1 | 12.2 | 1.7 | 94.1 | 8.0 | 1.4 |
| 117.6 | 47.6 | 7.4 | 117.6 | 19.4 | 2.1 | 117.6 | 14.2 | 1.4 |
| 141.2 | 77.4 | 13.8 | 141.2 | 37.2 | 5.8 | 141.2 | 24.7 | 3.1 |
| 174.1 | 207.8 | 42.3 | 177.9 | 83.3 | 11.0 | 174.9 | 72.7 | 13.7 |

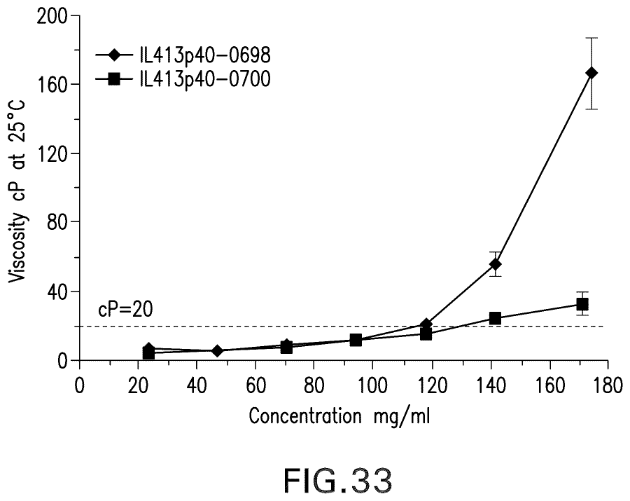

Similarly, DLS viscosity analysis shows that IL413p40-0700, which contains the E93K-bearing anti-IL-4 domain IL4-1040, has decreased viscosity relative to IL413p40-0698, which has the anti-IL-4 domain IL4-0749 (Table 49). This result confirms that mutations that resulted in improvements in viscosity in individual binding domains in a standard IgG structure can be translated to a Tri-Fab-Fc format. FIG. 33 shows that IL413p40-0700 was measured to have lower viscosity than of IL413p40-0698 at concentrations as high as 170 mg/mL.

TABLE 49
The E(L93K) amino acid substitution within the IL-4 binding
domain reduces the viscosity of the IL413p40-0700 Tri-
Fab-Fc as demonstrated using the DLS method.

| | |
|---|---|
| IL413p40-0698 | IL413p40-0700 (+E93K) |

| Concen- | | | Concen- | | |
|---|---|---|---|---|---|
| tration | Viscosity | Std. | tration | Viscosity | Std. |
| mg/mL | cP | Dev | mg/mL | (cP) | Dev |
| 23.5 | 5.5 | 0.8 | 23.5 | 4.5 | 0.7 |
| 47.1 | 5.1 | 0.8 | — | — | — |
| 70.6 | 7.7 | 1 | 70.6 | 6.8 | 1 |
| 94.1 | 11.4 | 1.2 | 94.1 | 10.6 | 2.9 |
| 117.6 | 20.6 | 2 | 117.6 | 15.1 | 2.9 |
| 141.2 | 55.5 | 7.2 | 141.2 | 21.1 | 3.8 |
| 174.1 | 166.5 | 21 | 170.1 | 37 | 7.4 |

### Example 45

### Evaluation of Pharmacokinetics for IL13433 Tri-Fab-Fc Variants and IL413TSLP-1024 Tri-Fab-Fc in the Tg32 Transgenic Mouse Model and Cynomolgus Monkeys

The human FcRn (hFcRn) transgenic 32 (Tg32) homozygous mouse is an in vivo tool for prediction of antibody human clearance (CL) (7). This assessment was conducted to gauge how similar the Tri-Fab-Fc variants were relative to an anti-IL-33 LS containing antibody that has established human PK. In brief, 6-10 week-old mice (Jackson Labs, #014565) received a single 5 mg/kg IV dose, and plasma samples were collected out to 8 weeks (4 mice/group). The study was conducted at Pfizer, Inc., and was designed and executed within accordance of the Animal Use Protocol and adherence to the Pfizer institutional animal care and use committee regulations. Quantitative analysis of plasma samples was conducted using a ligand binding assay (generic human IgG assay format) developed on the Gyrolab® platform.

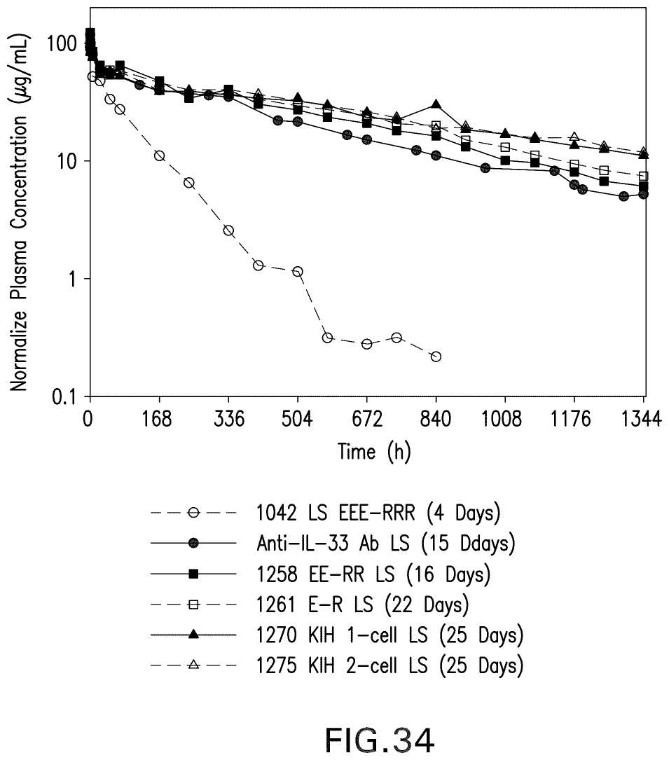

Results indicate that predicted clinical pharmacokinetic parameters for Tri-Fab-Fc variants IL413TSLP-1024, IL13433-1258, IL13433-1261, IL13433-1270 and IL13433-1275 engineered with LS mutations to extend the half-life were comparable to those of the anti-IL-33 antibody harboring LS mutations and have similar pharmacokinetic parameters in the Tg32 mouse (FIG. 34 and Table 50). Further, these findings suggest that Tri-Fab-Fc molecules with favorable pharmacokinetic profiles can be engineered using different modalities prepared using dual cell or single cell approaches that were engineered with charge-based (CB) or Knob-into-Hole Fc heterodimerization mutations (FIG. 34 and Table 50). Interestingly, IL13433-1042 exhibited an unexpectedly short half-life, which suggests that the P(H241)R mutation within the hinge region was responsible for the rapid clearance since IL13433-1258 (which has the wild-type sequence at H241 but is otherwise identical to IL13433-1258, except for a single mutation in the anti-IL-4 domain) has pharmacokinetic parameters similar to the control anti-IL-33 antibody with LS (FIG. 34 and Table 50). Similarly, IL413p40-0700, which carries the P(H241)R mutation like EL13433-1042 and is identical to IL13433-1258 except for the presence of p40-0003 in the Fab3 position, also exhibited rapid clearance, further underscoring the importance of P(H241)R.

Pharmacokinetic parameters for FL13433-1258 and IL413TSLP-1024 were also determined in Cynomolgus monkeys. L13433-1258 or IL413TSLP-1024 was administered to 6 female cynomolgus monkeys as single IV bolus dose per molecule, with dose levels ranging from 0.03 mg/kg to 300 mg/kg. Blood samples were collected pre-dose and post-dose to 1680 hours. The study was conducted at UL Lafayette-New Iberia Research Center (New Iberia, LA 70560) and executed within accordance of the Animal Use Protocol. Quantitative analysis of plasma samples was conducted using a ligand binding assay (generic human IgG assay format) developed on the Gyrolab® platform. Non-compartmental analysis was performed using Phoenix 64 Software (build 8.0.0.3176). The mean terminal half-life for IL13433-1258 and IL413TSLP-1024 was estimated to be 12 days and 14 days, respectively in cynomolgus monkeys.

TABLE 50
Terminal Half-lives Determined for LS Engineered Tri-Fab-Fc Variants as
Compared to an Anti-IL-33 Antibody Harboring LS Mutations in Tg32 mice.

| | | | | | Terminal |
|---|---|---|---|---|---|
| | Expression | Binding Domain/Orientation | | | Half-life |
| Sample | method/Fc format | Fab1 | Fab2 | Fab3 | (Days) |
| IL-33 Antibody | IgG, LS | NR | NR | NR | 15 |
| IL13433-1042 | Dual Cell: | IL13- | IL4- | IL33-0726 | 4 |
| Tri-Fab-Fc | CB: EEE/RRR, LS | 0001 | 0749 | native Fab | |
| | | mFd | native | | |
| | | | Fab | | |
| IL413p40-0700 | Dual Cell: | IL13- | IL4- | P40-0003 | 4.4 |
| Tri-Fab-Fc | CB: EEE/RRR, LS | 0001 | 1040 | native Fab | |
| | | mFd | native | | |
| | | | Fab | | |
| IL13433-1258 | Dual Cell: | IL13- | IL4- | IL33-0726 | 16 |
| Tri-Fab-Fc | CB: EE/RR, LS | 0001 | 1040 | native Fab | |
| | | mFd | native | | |
| | | | Fab | | |
| IL13433-1261 | Dual Cell: | IL13- | IL4- | IL33-0726 | 22 |
| Tri-Fab-Fc | CB: E/R, LS | 0001 | 1040 | native Fab | |
| | | mFd | native | | |
| | | | Fab | | |
| IL13433-1270 | Single Cell: KiH, LS | IL13- | IL4- | IL33-0726 | 25 |
| Tri-Fab-Fc | | 0001 | 1040 | S1 | |
| | | mFd | S1rev | | |
| IL13433-1275 | Dual Cell: KiH, No Cys | IL13- | IL4- | IL33-0726 | 25 |
| Tri-Fab-Fc | in CH3, LS | 0001 | 1040 | Native Fab | |
| | | mFd | native | | |
| | | | Fab | | |
| IL413TSLP- | Dual Cell: | IL13- | IL4- | TSLP-0875 | 18 |
| 1024 | CB: EE/RR, LS | 0001 | 1040 | | |
| | | mFd | native | | |
| | | | Fab | | |

CB = charge-based; KiH = Knob-into-Hole; mFd = modified Fd chain; LS = Leu/Ser half-life extension mutations; NR = not relevant.
IL33 Antibody is IL33-158LS (SEQ ID NO: 244 SEQ ID NO: 209 of WO17187307))

### Example 46

### Bioactivity Assessment of IL-4/13/33 Tri-Fab-Fc Variants Produced Using Different Tri-Fab-Fc Modalities

To determine whether the different modalities, geometry or production method utilized for preparing Tri-Fab-Fc molecules adversely impacted the potent cytokine neutralization ability of the binding domains, bioassays were performed to measure bioactivity against each cytokine. Neutralization activity of the individual Fab binding domain within the Tri-Fab-Fc modality should be ˜50% less that the parental antibody variant in standard IgG format since binding reflects monovalent versus bivalent neutralization ability.

The CD23 expression assay with primary human monocytes was used to determine neutralization potency for both the IL-4 and IL-13 cytokines. For IL-4 induced CD23 expression, mononuclear cells were isolated from human peripheral blood by layering over Histopaque (Sigma Aldrich). Cells were washed into RPMI medium containing 10% heat-inactivated Fetal Calf Serum (FCS), 50 U/mL penicillin, 50 μg/mL streptomycin, 2 mM L-glutamine, and plated in a 48-well tissue culture plate (Costar/Corning). Recombinant human IL-4 was added at dilutions ranging from 100 to 0.01 ng/mL. For assays testing Tri-Fab-Fc inhibition of cytokine responses, 0.25 ng/mL IL-13 or 0.1 ng/mL IL-4 was added along with dilutions of the Tri-Fab-Fc or antibodies ranging from 100 to 0.8 pM. Cells were incubated overnight at 37° C. in a 5% CO2 incubator. The next day, cells were harvested from wells using non-enzymatic Cell Dissociation Solution (Sigma Aldrich), and then washed into ice-cold PBS containing 1% BSA. Cells were incubated with phycoerythrin (PE)-labeled antibody to human CD23 (BD Biosciences), and Cy-Chrome-labeled antibody to human CD11b (BD Biosciences). Monocytes were gated based on high forward and side light scatter, and expression of CD11b. CD23 expression on monocytes was determined by flow cytometry using a flow cytometer (BD Biosciences), and the percentage of CD23-positive cells was analyzed with CellQuest software (BD Biosciences). Because the CD23 expression assay is run with human peripheral blood, the monocyte CD23 expression assay shows subtle variations in response based on donor. Data are expressed as % maximal response, which typically ranged from 65-85% CD23+ monocytes.

For IL-33 neutralization assays, HEK-Blue™ IL-33 Cells (Invivogen) are a HEK293-based cell line engineered to lack TNF and IL-1 signaling and stably express both IL1 RL1 and a NF-κB/AP-1-inducible secreted embryonic alkaline phosphatase (SEAP) reporter gene. Upon IL-33 stimulation, these cells secrete SEAP, which can subsequently be quantitated using a colorimetric assay to assess activity of IL-33. HEK-Blue™ IL-33 Cells were maintained in DMEM (Gibco 11995-085) supplemented with 1× pen/strep/glu (Invitrogen 10378-016), 10% heat-inactivated FBS (Gibco 16140-171), 10 μg/mL blastocidin (Invitrogen A11139-03), 300 μg/mL zeocin (Invitrogen R25001) in a 37° C. incubator with 5% CO₂. Prior to assay, cells were released from maintenance flasks with trypLE (Gibco), washed, and resuspended at 10⁶ cells/mL. Cells were then seeded at 5×10⁴ cells/well in assay plates (Falcon 353072). A stock solution of recombinant human IL-33 (R&D Systems 3625-IL) at 100 μg/mL was diluted 1:100 by adding 1.5 μL of the IL-33 stock solution to 1.5 μL DTT (Sigma 646563) and 147 μL of media. DTT addition prevents redox-mediated inactivation of IL-33 which would prohibit downstream readouts. Alternatively, recombinant IL-33 mm2 Cys, a constitutively active mutant of IL-33, was used at a final concentration of 0.1 ng/mL, however this assay readout is less dynamic than that with reduced IL-33. 25 μL of the test Tri-Fab-Fc dilutions were added to the 50 μL of cells in each well followed by 25 μL of the diluted IL-33 mixture for a final concentration of 0.1 ng/mL of IL-33. Cells were stimulated for approximately 20 hours in a 37° C. incubator with 5% CO₂, at which time 75 μL of media was removed from each well of the culture plate for SEAP quantitation. 160 μL of QUANTI-Blue reagent (Invivogen) was added to each well of an assay plate (Falcon 353072). 40 μL of the cell-conditioned medium was added to each well and the plate was returned to 37° C. incubator for approximately 3 hours. SEAP activity was then assessed using a spectrophotometer (Spectramax M5e) at 650 nm. Tri-Fab-Fc activity was assessed by the ability to suppress IL-33-induced SEAP activity.

The combined results from the bioactivity evaluation for ability of the Tri-Fab-Fc to effectively neutralize IL-4, IL-13 and IL-33 demonstrate that different Tri-Fab-Fc modalities produced using either the single cell or dual cell approach were able to confer expected potency from the parental antibody (Table 51). Further, if modification of the binding domains resulted in loss of neutralization activity for that antibody variant, then this translated to the Tri-Fab-Fc molecule and was more significant due to the monovalent interaction with the cytokine. Specifically, the IL4-0157 variant designed to remove an isomerization liability in CDRL1, lower T cell epitope content in CDRs and reduce viscosity (EXAMPLE 5) when incorporated into the IL13433-0607 Tri-Fab-Fc exhibited a substantial loss (˜6.5-fold) in IL-4 neutralization ability relative to the Tri-Fab-Fc variants harboring the parental 14-0002 Fab (compare L13433-0607 to IL13433-0006, Table 51). Another observation is that the CH1-Cκ swap used to engineer the IL-13 binding domain as Fab2 in the FL13433-0021 Tri-Fab-Fc appeared to marginally lower (˜2-fold) IL-13 neutralization ability relative to that in the Tri-Fab-Fc variant IL-13433-0005 which the Fab2 was engineered using the Si electrostatic complementary mutations (Table 51). Further, when inclusion of the SS elbow in the Fab2 IL-13 binding domain was engineered into IL13433-0021 resulting in IL13433-0022, this variant exhibited slightly more potent IL-13 neutralization capability with an IC₅₀ value of 17.5 pM versus 27.2 pM (Table 51).

TABLE 51
Potency of Cytokine Neutralization for IL-4/IL-13/IL-33 Tri-Fab-Fc Variants Prepared
using Different Tri-Fab-Fc Designs

| | | | | | Potency of Cytokine Neutralization [pM] | | | |
|---|---|---|---|---|---|---|---|---|
| | | | | | | | | IL-33 mm2 |
| | | | | | | | IL-33 WT | Cys |
| | | | | | | | Activation | Activation |
| | | | | | IL-4 | IL-13 | of | of |
| | Expression cell | Binding | | | Induced | Induced | SEAP | SEAP |
| Tri- | number/Fc | Domain/Orientation | | | CD23 | CD23 | Reporter | Reporter |
| Fab-Fc | format | Fab1 | Fab2 | Fab3 | Expression | Expression | Cells | Cells |
| IL13433- | Single cell, | IL33- | IL13- | IL4- | 3.2 | 12.5 | ND | 45* |
| 0005 | KiH | 0232 | 0001 | 0002 | | | | |
| | | mFd | S1rev | S1 | | | | |
| IL13433- | Single cell, | IL33- | IL4- | IL13- | 10.3 | 9.01 | 112 | 158, 40* |
| 0006 | KiH | 0232 | 0002 | 0001 | | | | |
| | | mFd | S1rev | S1 | | | | |
| IL13433- | Single cell, | IL33- | IL13- | IL4- | 6.1 | 27.2 | ND | 255 |
| 0021 | KiH | 0232 | 0001 | 0002 | | | | |
| | | S1 | CkS | S1rev | | | | |
| IL13433- | Single cell, | IL33- | IL13- | IL4- | 4.5 | 17.5 | ND | 251 |
| 0022 | KiH | 0232 | 0001 | 0002 | | | | |
| | | S1 | CkS- | S1rev | | | | |
| | | | SS | | | | | |
| | | | elbow | | | | | |
| L13433- | Single cell, | IL33- | IL4- | IL13- | 66.9 | 20.3 | ND | 182 |
| 0607 | KiH, LS | 0224 | 0157 | 0271 | | | | |
| | | mFd | S1rev | S1 | | | | |
| IL13433- | Dual Cell: | IL13- | IL4- | IL33- | 7.1 | 12.4 | 28 | ND |
| 1042 | EEE/RRR, | 0001 | 0749 | 0726 | | | | |
| | LS | mFd | native | native | | | | |
| | | | Fab | Fab | | | | |
| IL13433- | Dual Cell: | IL13- | IL4- | IL33- | 13.0 | 9.35 | 13 | ND |
| 1258 | EE/RR, LS | 0001 | 1040 | 0726 | | | | |
| | | mFd | native | native | | | | |
| | | | Fab | Fab | | | | |
| IL13433- | Dual Cell: | IL13- | IL4- | IL33- | 13.9 | 8.97 | 21 | ND |
| 1261 | E/R, LS | 0001 | 1040 | 0726 | | | | |
| | | mFd | native | native | | | | |
| | | | Fab | Fab | | | | |
| IL13433- | Single | IL13- | IL4- | IL33- | 16.5 | 7.58 | 12 | ND |
| 1270 | Cell: KiH, | 0001 | 1040 | 0726 | | | | |
| | LS | mFd | S1rev | S1 | | | | |
| IL13433- | Dual Cell: | IL13- | IL4- | IL33- | 14.9 | 8.88 | 13 | ND |
| 1275 | KiH, No | 0001 | 1040 | 0726 | | | | |
| | Cys in | mFd | native | native | | | | |
| | CH3, LS | | Fab | Fab | | | | |
| IL13433- | Single | IL13- | IL4- | IL33- | 13.7 | 9.13 | 13 | ND |
| 1279 | Cell: | 0001 | 1040 | 0726 | | | | |
| | KiH, no | mFd | S1rev | S1 | | | | |
| | engineered | | | | | | | |
| | Cys in | | | | | | | |
| | CH3, LS | | | | | | | |

*IL-33 neutralization IC₅₀ values determined in the same experiment for IL13433-0005 and IL13433-0006. CH1-C_{K} swap = Fab has CH1 and Kappa constant domains exchanged; KiH =Knob-into-Hole; mFd = modified Fd chain; LS = Leu/Ser half-life extension mutations; ND = not determined.

### Tri-Fab-Fc Characterization: IL-4 Binding Domain

### Example 47

### IL-4 Neutralization Activity of mAbs IL4-1285 and IL-4-1305 in Whole Blood

IL4-1285 was the template variant used for affinity engineering efforts. IL4-1305 is an affinity improved variant. mAb IL4-0002 is a variant of IL4-1305 with a germline JH segment. IL4-1040 was engineered for improved biophysical properties. The IL4-1040 binding domain (Fab) was incorporated into trispecifics IL13433-1258, IL413TSLP-1024, and IL413P40-0705.

IL-4 binding to type I (IL-4Rα/γ common), or IL-4 or IL-13 binding to type II (IL-4Rα/IL-13Rα1) receptor triggers the phosphorylation and nuclear translocation of the transcription factor, STAT6 (28). B cells and monocytes express type II (IL-4Rα/IL-13Rα1) receptors and respond to both IL-4 and IL-13. In contrast, T cells express type I (IL-4Rα/γ common) but not type II receptor, and so respond to IL-4 but not IL-13 (28, 29). The pSTAT6 assay described in EXAMPLE 3 was used to confirm that IL4-1305 effectively inhibited IL-4 responses of B cells, T cells, and monocytes as gated subpopulations in human whole blood (Table 52). The demonstration that IL4-1305 blocks STAT6 phosphorylation in peripheral blood T cells, in addition to responses in monocytes and B cells, supports that IL-4 responses through both type I and type II receptors are inhibited.

TABLE 52
Cytokine Neutralization Activity
of IL-4 Antibodies in Whole Blood

| | | IC₅₀ for STAT6 Phosphorylation | | |
|---|---|---|---|---|
| | Cytokine | in Gated Cell Types (pM) ^{a} | | |
| Antibody | Target ^{b} | Monocytes | B Cells | T cells |
| IL4-1285 | IL-4 | 320 | 210 | 180 |
| IL4-1305 | IL-4 | 100 | 73 | 75 |

^{a} Human whole blood was challenged with IL-4 for 30 minutes at 37° C. Cells were fixed, permeabilized, and stained with PE-labeled antibody to pSTAT6, along with APC labeled anti CD3, PerCP Cy5.5 labeled anti-CD20, and FITC labeled anti-CD14. Within the lymphocyte fraction, B cells were identified as CD20+ CD3−, and T cells as CD20− CD3+. Monocytes were identified as CD14+ cells with intermediate side light scatter (SSC).
^{b} IL-4 was tested at 80% effective concentration (EC80) for each cell type.

### Example 48

### IL-4 Neutralization Activity of mAbs IL4-1040 and IL4-0002, and Trispecifics IL13433-1258, IL413TSLP-1024, IL413TSLP-1028, IL413TSLP-1037 and IL413P40-0705 in the Monocyte CD23 Bioassay

Trispecifics IL13433-1258, IL413TSLP-1024, IL413TSLP-1028, IL413TSLP-1037 and IL413P40-0705 all utilize the property-engineered IL-4 binding domain, IL4-1040. mAb IL4-1040 was derived from affinity-improved anti-IL-4 clone IL4-1305, with amino acid substitutions introduced to eliminate an in silico predicted T cell epitope, remove an isomerization liability and improve viscosity. mAb IL4-0002 is a variant of IL4-1305 with a germline JH segment.

IL-4 and IL-13 drive the expression of activation markers, including the low affinity IgE receptor, CD23, on human peripheral blood monocytes (30, 31). IL-4 neutralization activity was evaluated using the monocyte CD23 expression bioassay, performed as described in EXAMPLE 4. Table 53 shows the IL-4 neutralization profile for trispecifics IL13433-1258, IL413TSLP-1024, IL413TSLP-1028, IL413TSLP-1037 and IL413P40-0705, compared with constituent IL-4 binding domain, 14-1040, and precursor affinity improved 14-0002. 14-1040 was engineered for improved biophysical properties over IL4-0002. Because mAb IL4-1040 is bivalent and the trispecific contains monovalent Fab for cytokine binding, the trispecifics are expected to have reduced neutralization capacity on a molar basis. For IL-4, trispecific IL13433-1258 had ˜3.1× lower potency than mAb IL4-1040, IL413TSLP-1024 had ˜2.5× lower potency than mAb IL4-1040, and IL413P40-0705 had ˜3.2× lower potency than mAb IL4-1040. Nevertheless, the trispecifics retained potent IL-4 neutralization activity. IL13433-1258, IL413TSLP-1024, IL413TSLP-1028, IL413TSLP-1037 and IL413P40-0705 inhibited IL-4 bioactivity with IC₅₀ values of 10.0 pM, 8.27 pM, 12.8 pM, 10.5 pM and 10.3 pM, respectively (Table 53).

TABLE 53
IL-4 Neutralization Activity (IC₅₀; pM) of Trispecifics
IL13433-1258, IL413TSLP-1024, IL413TSLP-1028, IL413TSLP-1037
and IL413P40-0705, and mAbs IL4-1040 and IL4-0002

| | Antibody or | | |
|---|---|---|---|
| Cytokine | Trispecific | | IC₅₀ (pM) |
| rHu IL-4 | IL13433-1258 | Median | 10.0 |
| | | s.e.m. | 0.71 |
| | | N | 13 |
| | IL134TSLP-1024 | Median | 8.27 |
| | | s.e.m. | 1.13 |
| | | N | 7 |
| | IL413TSLP-1028 | Median | 12.8 |
| | | s.e.m. | |
| | | N | 1 |
| | IL413TSLP-1037 | Median | 10.5 |
| | | s.e.m. | |
| | | N | 1 |
| | IL413P40-0705 | Median | 10.3 |
| | | s.e.m. | 0.97 |
| | | n | 3 |
| | IL4-1040 | Median | 3.23 |
| | | s.e.m. | |
| | | n | 1 |
| | IL4-0002 | Median | 2.09 |
| | | s.e.m. | 0.16 |
| | | n | 18 |

Human peripheral blood mononuclear cells were incubated overnight at 37° C. with recombinant human IL-4 (0.1 ng/ml; R&D Systems), along with dilutions of trispecifics IL13433-1258, IL413TSLP-1024, IL413TSLP-1028, IL413TSLP-1037 or IL413P40-0705, or mAbs IL4-1040 and IL4-0002. CD23 expression on gated monocytes was quantitated by flow cytometry, and the percentage of CD23-positive cells determined.

### Example 49

### IL-4 Neutralization Activity of mAbs IL4-1040 and IL4-0002, and Trispecifics IL13433-1258, IL413TSLP-1024, and IL413P40-0705 in the Whole Blood CD23 Bioassay

The CD23 expression assay was adapted to whole blood collected into sodium heparin anti-coagulant. In a deep-well culture plate, 80 μl whole blood was added with IL-4 or IL-13, along with the trispecific at concentrations indicated above, for a total volume of 100 ul. The plate was incubated overnight at 37° C. degrees 5% CO2, then incubated an additional 30 min at 37° C. with anti-CD11 b-PE-Cy5 and anti-CD23-PE in PBS/1% BSA (BD Biosciences). Cells were lysed with Optilyse solution (BD Biosciences), washed with PBS, and analyzed by flow cytometry. Experiments confirmed that the trispecifics IL13433-1258, IL413TSLP-1024, and IL413P40-0705 block cytokine activity in human whole blood in addition to human peripheral blood monocytes (Table 54).

### Example 50

### Neutralization Activity of Trispecifics IL13433-1258, IL413TSLP-1024, and IL413P40-0705 Against Human and Cynomolgus Monkey IL-4

To confirm antibody activity against cynomolgus monkey cytokines, neutralization activity of trispecifics IL13433-1258, IL413TSLP-1024, and IL413P40-0705 was tested in the monocyte CD23 expression bioassay with peripheral blood monocytes or whole blood. Bioactivity of human or cynomolgus monkey IL-4 was efficiently inhibited by each trispecific. For each, neutralization activity against cynomolgus IL-4 was ˜ 1.1-1.4× reduced compared to the human cytokine, when assayed on monocytes (Table 54).

TABLE 54
Neutralization Activity of Trispecific IL13433-
1258 against Human or Cynomolgus Monkey IL-4

| | | Peripheral Blood | | | |
|---|---|---|---|---|---|
| | | Monocytes | | Human Whole Blood | |
| | | (IC₅₀; pM) | | (IC₅₀; pM) | |
| | | | Cynomolgus | | Cynomolgus |
| | | Human | Monkey | Human | Monkey |
| IL13433-1258 | Median | 10.0 | 13.60 | 23.25 | 32.10 |
| | s.e.m. | 0.71 | 0.80 | 3.59 | 4.09 |
| | N | 13 | 7 | 6 | 6 |
| IL413TSLP- | Median | 8.27 | 10.12 | 23.55 | 16.45 |
| 1024 | s.e.m. | 1.13 | 0.76 | 4.47 | 4.39 |
| | N | 7 | 7 | 6 | 6 |
| IL413P40- | Median | 10.3 | 11.2 | 23.2 | 17.24 |
| 0705 | s.e.m. | 0.97 | 1.49 | 0.94 | 0.3 |
| | n | 3 | 3 | 3 | 3 |

Human whole blood or isolated mononuclear cells were incubated overnight at 37° C. with recombinant human IL-4 (0.1 ng/ml; R&D Systems) or cynomolgus monkey IL-4 (0.25 ng/ml; Pfizer), along with dilutions of trispecific IL13433-1258. CD23 expression on gated monocytes was quantitated by flow cytometry, and the percentage of CD23-positive cells determined.

### Example 51

### Species Specificity of Anti-IL-4 mAb IL4-1305

To evaluate the species specificity of anti-IL-4 mAb IL4-1305, the ability of mouse, rat, dog, rabbit, sheep, and cynomolgus monkey IL-4 to compete for antibody binding to recombinant human IL-4 was tested. The percentage of amino acid homology between each species IL-4 and the human sequence is shown in Table 55. IL4-1305 bound to human and cynomolgus monkey IL-4, but not to IL-4 from the other species tested (Table 55).

TABLE 55
Competition by Test Species IL-4 for
Human IL-4 Binding to mAb IL4-1305

| | | Amino Acid | |
|---|---|---|---|
| | | Sequence | Competition |
| | | Identity | for binding |
| IL-4 | Accession # | (%) ^{a} | to IL4-1305 ^{b} |
| Human | M23442 | 100 | +++ |
| Cyno | AB000515 | 92 | ++ |
| Dog | EF095771 | 49 | − |
| Sheep | NM_001009313 | 57 | − |
| Rabbit | DQ852343.1 | 54 | − |
| Rat | NM_201270 | 43 | − |
| Mouse | NM_021283 | 41 | − |

^{a} The percentage identity between amino acid sequences of IL-4 and various human cytokines was compared using the Basic Local Alignment Search Tool (BLAST). GenBank accession numbers for the cytokine sequences are shown.
^{b} IL4 from the indicated species was assayed by ELISA for ability to compete with biotinylated human IL4 for binding to IL4-1305. Recombinant human, mouse, rat, dog, and rabbit IL 4 were purchased from R&D Systems (Minneapolis, MN). Recombinant cynomolgus monkey and sheep IL-4 were prepared by Pfizer, Cambridge, MA).

### Example 52

### Binding of IL13433-1258, IL413TSLP-1024, and IL413P40-0705 to Human, Cynomolgus Monkey, Mouse, and Rat IL-4 by Surface Plasmon Resonance

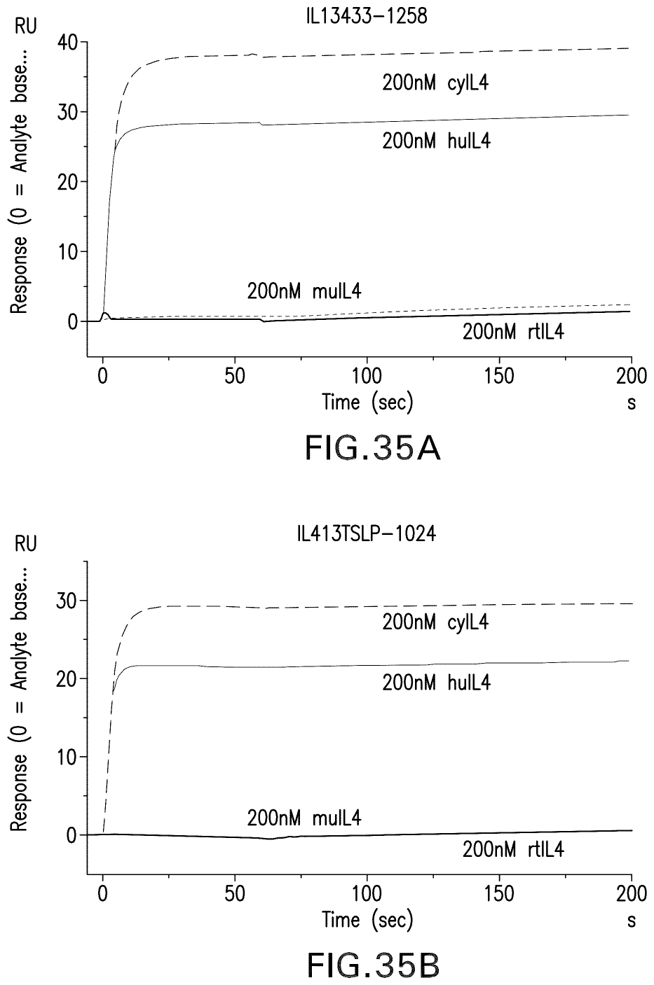

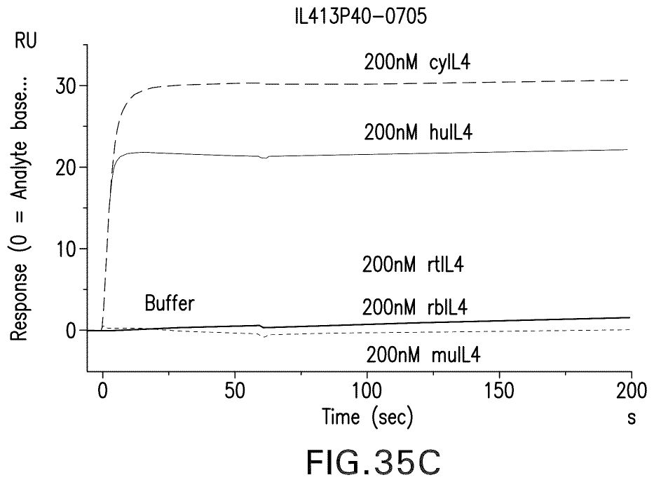

Cross-species studies using surface plasmon resonance (SPR) characterized the binding of trispecifics IL13433-1258, IL413TSLP-1024, and IL413P40-0705 to human, cynomolgus monkey, mouse, and rat IL-4. All experiments were performed using a Biacore 8K+instrument (GE HealthCare, Marlborough, MA). Anti-human Fc (Jackson Immunoresearch) or anti-mouse Fc (GE HealthCare, Marlborough, MA) was immobilized on a CM5 sensor chip using a standard amine coupling protocol provided by GE HealthCare. Trispecifics IL13433-1258, IL413TSLP-1024, or IL413P40-0705, a control antibody to mouse (clone 1D11; BD Biosciences), or a control antibody to rat (MAB5041; R&D Systems) IL-4, was captured followed by flow of human (Pfizer-Syngene), cynomolgus monkey (Kingfisher Biotech), mouse (R&D Systems), or rat (R&D Systems) IL-4 at a concentration of 200 nM. The association and dissociation phases were 60 seconds and 300 seconds, respectively. At the end of the dissociation phase, the surface containing anti-human or anti-mouse Fc was regenerated using one 30 second pulse of 3M MgCl₂ followed by one 30 second pulse of 10 mM glycine pH 1.7. FIG. 35 shows that IL13433-1258, IL413TSLP-1024, and IL413P40-0705 bind to human and cynomolgus monkey IL-4 at a concentration of 200 nM, but do not bind to mouse or rat IL-4. IL413P40-0705 was also tested against rabbit IL-4, and did not bind.

### Example 53

### Binding Affinity of IL13433-1258, IL413TSLP-1024, or IL413P40-0705 to Human and Cynomolgus Monkey IL-4 by KinExA

A Kinetic exclusion assay (KinExA) instrument (model 3200, Sapidyne) was used to determine the binding affinity of trispecifics IL13433-1258, IL413TSLP-1024, or IL413P40-0705 to human IL-4 and cynomolgus monkey IL-4. Samples were prepared in PBS containing 0.1% sodium azide and 1.0 mg/ml BSA. The fixed antigen assay method was used to determine binding affinity. Trispecific IL13433-1258 was serially diluted 2-fold from 2 nM to 12.21fM and titrated with human IL-4 (Pfizer-Syngene) or cynomolgus monkey IL-4 (Kingfisher Biotech) with concentrations that were kept constant at 10 pM and 50 pM for human IL-4 and 20 pM and 200 pM for cynomolgus monkey IL-4. Each trispecific and IL-4 cytokine were equilibrated for at least 72 hours at room temperature, then passed through a flow cell containing Polymethylmethacrylate (PMMA) beads coated with anti-hIL-4 Ab-0002 (Pfizer) that contains the same IL-4 binding domain as the trispecifics. A non-competing rat anti-IL-4 antibody (Abnova MAB3293) captured the free IL-4 and was detected with 1 ug/ml Alexa Fluor 647-conjugated F(ab′)2 goat anti-rat IgG (H+L) (Jackson Immunoresearch). Data analysis was performed with KinExA Pro software version 4.3.11 (Sapidyne). The ‘affinity standard’ model was used to analyze the data and determine the K_{D} and active concentration of the IL-4 cytokine. The ‘drift correction’ fitting option was used when responses varied between replicate injections. Two curves were obtained in independent experiments and analyzed using the ‘n-curve analysis’ tool to obtain global best fit values for the K_{D} and active concentration of IL-4 cytokine. The software reports each best fit value along with a 95% confidence interval. Results showed that IL13433-1258 binds to cynomolgus monkey IL4 about 6.2-fold weaker than the human IL-4 affinity value (Table 56). IL413TSLP-1024 binds to cynomolgus monkey IL-4 within 2-fold of the human IL-4 affinity value (Table 56). IL413P40-0705 binds to cynomolgus monkey IL-4 within 1.2-fold of the human IL-4 affinity value (Table 56).

TABLE 56
Affinity of IL13433-1258 Binding to Human
or Cynomolgus Monkey IL-4 by KinExA

| | | | Cynomolgus |
|---|---|---|---|
| | | Human IL-4 | Monkey IL-4 |
| Trispecific | | (K_{D}; pM) | (K_{D}; pM) |
| IL13433-1258 | Mean | 0.477 | 2.960 |
| | s.d. | 0.012 | 0.068 |
| IL413TSLP-1024 | Mean | 0.323 | <0.661 |
| | s.d. | 0.017 | |
| IL413P40-0705 | Mean | 0.739 | 0.918 |
| | s.d. | | |

Data shown are mean +/− s.d. using recombinant forms of human IL-4 (R&D Systems), or cynomolgus monkey IL-4 (Pfizer).

### Example 54

### IL-4 Neutralization Activity of Trispecific IL13433-1258 IL413TSLP-1024 and IL413P40-0705 Compared to Dupilumab

A leading molecule aimed at neutralizing the bioactivity of IL-4 and IL-13 in atopic dermatitis is anti-IL-4R dupilumab (Dupixent®; Sanofi/Regeneron) (32), which was the first biologic to be approved for treatment of AD (33). Dupilumab targets the IL-4Rα chain shared by the IL-4 and IL-13 signaling complexes, and neutralizes the activity of both cytokines (34).

We compared the cytokine neutralization activity of trispecifics against dupilumab in the monocyte CD23 expression bioassay. The IL-4 neutralization activity of trispecifics IL13433-1258, IL413TSLP-1024, and IL413P40-0705 exceeded that of dupilumab by ˜10×, 13×, and 10×, respectively (Table 57).

TABLE 57
IL-4 Neutralization Activity of IL13433-1258,
IL413TSLP-1024, IL413P40-0705, and Dupilumab

| Cytokine | Antibody | | IC₅₀ (pM) ^{a} |
|---|---|---|---|
| rHu IL-4 | IL13433-1258 | Median | 10.0 |
| | | s.e.m. | 0.71 |
| | | n | 13 |
| | IL413TSLP- | Median | 8.27 |
| | 1024 | s.e.m. | 1.13 |
| | | n | 7 |
| | IL413P40- | Median | 10.3 |
| | 0705 | s.e.m. | 0.9 |
| | | n | 3 |
| | Dupilumab | Median | 104.4 |
| | | s.e.m. | 9.85 |
| | | n | 6 |

^{a} Mononuclear cells isolated from human peripheral blood were incubated overnight at 37° C. with recombinant human IL-4 (0.1 ng/ml; R&D Systems), along with dilutions of trispecific or dupilumab. CD23 expression on gated monocytes was quantitated by flow cytometry, and the percentage of CD23-positive cells determined.

### Tri-Fab-Fc Characterization: IL-13 Binding Domain

### Example 55

### IL-13 Neutralization Activity of Anti-IL-13 mAbs IL13-1307 and IL13-0001

Anti-IL-13 template variant IL13-1307 was used for affinity engineering efforts. It inhibited the phosphorylation of STAT6 in human HT-29 epithelial cells in a 30 min assay, and neutralized IL-13-induced CD23 expression on human peripheral blood monocytes in a 24 hr assay (Table 58). Affinity engineered clone IL13-0001 was 29-fold more potent than IL13-1307 in the CD23 expression bioassay (Table 58). The IL13-0001 binding domain (Fab) was incorporated into trispecifics IL13433-1258, IL413TSLP-1024, and IL413P40-0705.

### Example 56

### Reactivity of mAb IL13-0001 Against Polymorphic Variant Human IL-13 (R110Q)

A polymorphic variant form of human IL-13 (R110Q), expressed at an allele frequency of approximately 20%, has been associated with increased concentration of serum IgE and increased risk of atopy (35, 36). IL13-0001 had comparable neutralization activity against both the non-polymorphic (R110), and polymorphic variant (Q110) forms of human IL-13 (Table 58). In addition to recombinant cytokine, IL13-0001 neutralized native human IL 13 derived from Th2 skewed, mitogen activated umbilical cord blood human T cells (Table 58).

TABLE 58
Cytokine Neutralization Activity of
IL-13 Antibodies in Cell-Based Assays

| | | | IC₅₀ (pM) | |
|---|---|---|---|---|
| | | | Monocyte | IC₅₀ (pM) |
| | | | CD23 | STAT6 |
| Cytokine | Antibody | | expression ^{a} | phosphorylation ^{b} |
| rHu | IL13-1307 | Median | 305.9 | 140 |
| IL-13 | | s.e.m. | 38.1 | 38.2 |
| | | n | 17 | 18 |
| | IL13-0001 | Median | 10.5 | 92.0 |
| | | s.e.m. | 1.3 | 8.1 |
| | | n | 17 | 15 |
| rHu | IL13-0001 | Median | 10.1 | 104.5 |
| IL-13 | | s.e.m. | 0.08 | 3.9 |
| R110Q | | n | 2 | 2 |
| Native | IL13-0001 | Median | | 79.0 |
| Hu IL-13 | | s.e.m. | | 4.2 |
| | | n | | 2 |

^{a} CD23 expression was assayed in human peripheral blood monocytes exposed to IL-13 (1 ng/ml) for 24 hours at 37° C.
^{b} STAT6 phosphorylation was assayed in HT-29 human epithelial cells exposed to IL-13 (1 ng/ml) for 30 min at 37° C.

### Example 57

### IL-13 Neutralization Activity of mAb IL13-0001 in Human Whole Blood

The pSTAT6 assay was used to confirm that IL13-0001 effectively inhibited IL-13 responses of B cells and monocytes as gated subpopulations in human whole blood (Table 59). In both B cells and monocytes, IL13-0001 displayed potent IL-13 neutralization activity (Table 59).

TABLE 59
Cytokine Neutralization Activity of IL13-0001 in Whole Blood

| | | | IC₅₀ for STAT6 Phosphorylation in | | |
|---|---|---|---|---|---|
| | Cytokine | | Gated Cell Types (pM) ^{a} | | |
| Antibody | Target ^{b} | | Monocytes | B Cells | T cells |
| IL13-0001 | IL-13 | Median | 525.0 | 66.5 | n.d. ^{c} |
| | | s.e.m. | 24.7 | 3.2 | |
| | | n | 2 | 2 | |

^{a} Human whole blood was challenged with IL-13 for 30 minutes at 37° C. Cells were fixed, permeabilized, and stained with PE-labeled antibody to pSTAT6, along with APC labeled anti CD3, PerCP Cy5.5 labeled anti-CD20, and FITC labeled anti-CD14. Within the lymphocyte fraction, B cells were identified as CD20+ CD3−, and T cells as CD20− CD3+. Monocytes were identified as CD14+ cells with intermediate side light scatter (SSC).
^{b} IL-13 was tested at 80% effective concentration (EC80) for each cell type.
^{c} n.d .: not detectable. T cells do not respond to IL-13.

### Example 58

### IL-13 Neutralization Activity of mAb IL13-0001 and Trispecifics IL13433-1258, IL413TSLP-1024, IL413TSLP-1028, IL413TSLP-1037 and IL413P40-0705

The IL13-0001 binding domain (Fab) was incorporated into trispecifics IL13433-1258, IL413TSLP-1024, IL413TSLP-1028, IL413TSLP-1037 and IL413P40-0705. IL-13 neutralization activity of trispecifics IL13433-1258, IL413TSLP-1024, IL413TSLP-1028, IL413TSLP-1037 and IL413P40-0705 was evaluated using the human monocyte CD23 expression bioassay (EXAMPLE 4) and compared to that of affinity-improved anti-IL-13 clone IL13-0001. Because mAb IL13-0001 is a bivalent IgG and the trispecific is monovalent for cytokine binding, the trispecific is expected to have reduced neutralization capacity on a molar basis. For IL-13 neutralization, IL13433-1258 had ˜2.7× lower potency than IL13-0001, IL413TSLP-1024 had ˜3.0× lower potency than IL13-0001, and IL413P40-0705 had ˜2.4× lower potency than IL13-0001 (Table 60). Nevertheless, the trispecifics retained potent IL-13 neutralization activity. IL13433-1258, IL413TSLP-1024, IL413TSLP-1028, IL413TSLP-1037 and IL413P40-0705 neutralized IL-13 bioactivity with IC₅₀ values of 11.74 pM, 12.98 pM, 12.9 pM, 13.1 pM and 10.4 pM, respectively (Table 60).

TABLE 60
IL-13 Neutralization Activity of Trispecific IL13433-
1258, IL413TSLP-1024, IL413TSLP-1028, IL413TSLP-
1037, IL413P40-09705, and mAb IL13-0001

| | Antibody or | | |
|---|---|---|---|
| Cytokine | Trispecific | | IC₅₀ (pM) |
| rHu IL-13 | IL13433-1258 | Median | 11.74 |
| | | s.e.m. | 0.50 |
| | | n | 13 |
| | IL413TSLP-1024 | Median | 12.98 |
| | | s.e.m. | 1.51 |
| | | n | 7 |
| | IL413TSLP-1028 | Median | 12.9 |
| | | s.e.m. | |
| | | N | 1 |
| | IL413TSLP-1037 | Median | 13.1 |
| | | s.e.m. | |
| | | N | 1 |
| | IL413P40-0705 | Median | 10.4 |
| | | s.e.m. | 0.3 |
| | | n | 3 |
| | IL13-0001 | Median | 4.38 |
| | | s.e.m. | 0.42 |
| | | n | 5 |

Human peripheral blood mononuclear cells were incubated overnight at 37° C. with recombinant human IL-13 (0.25 ng/ml; R&D Systems), along with dilutions of trispecifics IL13433-1258, IL413TSLP-1024, IL413TSLP-1028, IL413TSLP-1037, IL413P40-0705 or mAb IL13-0001. CD23 expression on gated monocytes was quantitated by flow cytometry, and the percentage of CD23-positive cells determined.

### Example 59

### IL-13 Neutralization Activity of mAbs IL4-1040 and IL4-0002, and Trispecifics IL13433-1258, IL413TSLP-1024, and IL413P40-0705 in the Whole Blood CD23 Bioassay

The CD23 expression assay was adapted to whole blood, as described in EXAMPLE 49. Experiments confirmed that the trispecifics IL13433-1258, IL413TSLP-1024, and IL413P40-0705 block cytokine activity in human whole blood in addition to human peripheral blood monocytes, using the CD23 expression assay adapted to whole blood format (Table 61).

### Example 60

### Neutralization Activity of IL13433-1258, IL413TSLP-1024, and IL413P40-0705 Against Human and Cynomolgus Monkey IL-13

The human monocyte CD23 expression bioassay demonstrated that trispecifics IL13433-1258, IL413TSLP-1024, and IL413P40-0705 retained neutralization activity against cynomolgus monkey IL-13. For IL13433-1258, neutralization activity against cynomolgus monkey IL-13 was ˜ 4.5× reduced compared to the human cytokine (Table 61). For IL413TSLP-1024, neutralization activity against cynomolgus monkey IL-13 was ˜2.4× reduced compared to the human cytokine (Table 61). For IL413P40-0705, neutralization activity against cynomolgus monkey IL-13 was ˜4.0× reduced compared to the human cytokine (Table 61).

TABLE 61
Neutralization Activity of IL13433-1258, IL413TSLP-1024, and
IL413P40-0705 against Human or Cynomolgus Monkey IL-13

| | | Peripheral Blood Monocytes | | Human Whole Blood | |
|---|---|---|---|---|---|
| | | (IC₅₀; pM) | | (IC₅₀; pM) | |
| | | | Cynomolgus | | Cynomolgus |
| | | Human | Monkey | Human | Monkey |
| IL13433-1258 | Median | 11.74 | 52.80 | 16.05 | 50.50 |
| | s.e.m. | 0.50 | 1.87 | 2.04 | 2.99 |
| | n | 13 | 7 | 6 | 6 |
| IL413TSLP-1024 | Median | 12.98 | 31.65 | 10.23 | 41.85 |
| | s.e.m. | 1.51 | 3.14 | 3.27 | 10.68 |
| | n | 7 | 8 | 6 | 6 |
| IL413P40-0705 | Median | 10.4 | 41.5 | 10.8 | 44.6 |
| | s.e.m. | 0.3 | 3.3 | 0.3 | 1.69 |
| | n | 3 | 3 | 3 | 3 |

Human whole blood or isolated mononuclear cells were incubated overnight at 37° C. with recombinant human IL-13 (0.25 ng/ml; R&D Systems) or cynomolgus monkey IL-13 (0.25 ng/ml; Pfizer), along with dilutions of trispecific IL13433-1258. CD23 expression on gated monocytes was quantitated by flow cytometry, and the percentage of CD23-positive cells determined.

### Example 61

### Species Specificity of Anti-IL-13, IL13-0001

To evaluate the species specificity of IL13-0001, the ability of mouse, dog, rabbit, sheep, and cynomolgus monkey IL 13 to compete for antibody binding to recombinant human IL-13 was tested. The percentage of amino acid homology between each species IL 13 and the human sequence is shown in Table 62. IL13-0001 bound to human, cynomolgus monkey, and sheep IL 13, but not to IL 13 from mouse, dog, or rabbit (Table 62).

TABLE 62
Competition by Test Species IL-13 for
Human IL-13 Binding to IL13-0001

| | | Amino Acid | Competition |
|---|---|---|---|
| | | Sequence | for binding |
| IL-13 | Accession # | Identity (%) ^{a} | to IL4-1305 ^{b} |
| Human | U31120 | 100 | +++ |
| Cyno | DQ676797 | 94 | +++ |
| Dog | NM_001003384 | 72 | − |
| Sheep | NM_001082594 | 70 | +++ |
| Rabbit | XM_002710092.2 | 66 | − |
| Mouse | NM_008355 | 59 | − |

^{a} The percentage identity between amino acid sequences of IL-13 and various human cytokines was compared using the Basic Local Alignment Search Tool (BLAST).
GenBank accession numbers for the cytokine sequences are shown.
^{b} IL-13 from the indicated species was assayed by ELISA for ability to compete with biotinylated human IL-13 for binding to IL13-0001. Recombinant human, mouse, rat, and dog IL 13 were purchased from R&D Systems (Minneapolis, MN). Recombinant sheep and rabbit IL-13 were produced by Pfizer Global Biotechnologies (PGBT, Cambridge, MA). Recombinant cynomolgus monkey IL 13 was purchased from Sino Biological (Beijing, China).

### Example 62

### Binding of IL13433-1258, IL413TSLP-1024, and IL413P40-0705 to Human, Cynomolgus Monkey, Mouse, and Rat IL-13 by Surface Plasmon Resonance

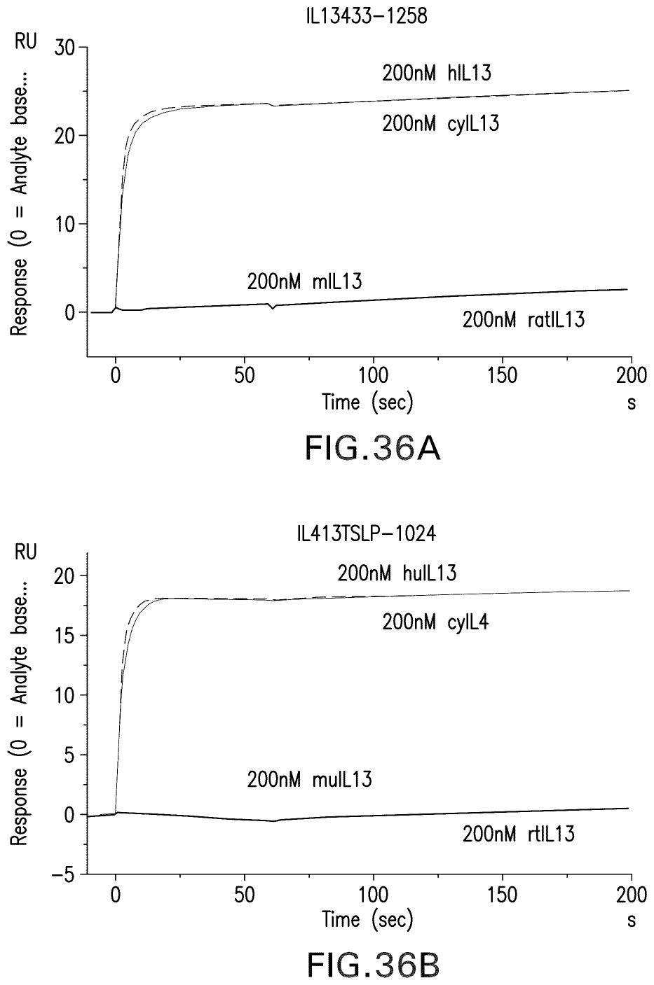

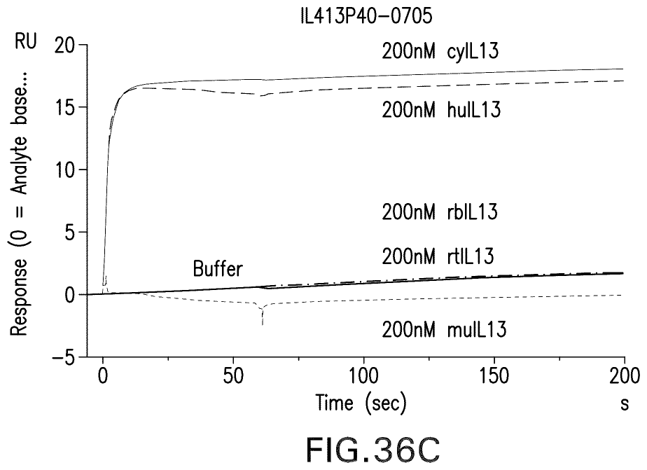

Cross-species studies using surface plasmon resonance (SPR) characterized the binding of IL13433-1258 to human, cynomolgus monkey, mouse, and rat IL-13. All experiments were performed using a Biacore 8K+ instrument (GE HealthCare, Marlborough, MA). Anti-human Fc (Jackson Immunoresearch) or anti-mouse Fc (GE HealthCare, Marlborough, MA) was immobilized on a CM5 sensor chip using a standard amine coupling protocol provided by GE HealthCare. Trispecifics IL13433-1258, IL413TSLP-1024, or IL413P40-0705, or a control antibody to mouse (MAB413; R&D Systems), or a control antibody to rat (MAB1945; R&D Systems) IL-13, was captured followed by flow of human (Pfizer), cynomolgus monkey (Pfizer), mouse (R&D Systems), or rat (R&D Systems) IL-13 at a concentration of 200 nM. The association and dissociation phases were 60 seconds and 300 seconds, respectively. At the end of the dissociation phase, the surface containing anti-human or anti-mouse Fc was regenerated using one 30 second pulse of 3M MgCl₂ followed by one 30 second pulse of 10 mM glycine pH 1.7. FIG. 36 shows that trispecifics IL13433-1258, IL413TSLP-1024, and IL413P40-0705 bind to human and cynomolgus monkey IL-13 at a concentration of 200 nM, and do not bind to mouse or rat IL-13. IL413P40-0705 was also tested against rabbit IL-13, and did not bind.

### Example 63

### Binding Affinity of IL13433-1258, IL413TSLP-1024, and IL413P40-0705 to Human and Cynomolgus Monkey IL-13 by KinExA

A Kinetic exclusion assay (KinExA) instrument (model 3200, Sapidyne) was used to determine the binding affinity of trispecifics IL13433-1258, IL413TSLP-1024, and IL413P40-0705 to human IL-13 and cynomolgus monkey IL-13. Samples were prepared in PBS containing 0.1% sodium azide and 1.0 mg/ml BSA. The fixed antigen assay method was used to determine binding affinity. Trispecific IL13433-1258 was serially diluted 2-fold from 600 pM to 12.21 fM and titrated with human IL-13 (Pfizer) or cynomolgus monkey IL-13 (Pfizer) with concentrations that were kept constant at 10 pM and 100 pM for human IL-13 and 5 pM and 30 pM for cynomolgus monkey IL-13. Trispecific IL13433-1258 and IL-13 cytokine were equilibrated for at least 72 hours at room temperature, then passed through a flow cell containing Polymethylmethacrylate (PMMA) beads coated with anti-hIL-13 Antibody-0271 (Pfizer) that contains the same IL-13 binding domain as the trispecifics. A non-competing mouse anti-IL-13 antibody MJ2-7 (Pfizer) captured the free IL-13 and was detected with 0.5 ug/ml Alexa Fluor 647-conjugated AffiniPure goat anti-mouse IgG (Jackson Immunoresearch). Data analysis was performed with KinExA Pro software version 4.3.11 (Sapidyne). The ‘affinity standard’ model was used to analyze the data and determine the K_{D} and active concentration of the IL-13 cytokine. The ‘drift correction’ fitting option was used when responses varied between replicate injections. Two curves were obtained in independent experiments and analyzed using the ‘n-curve analysis’ tool to obtain global best fit values for the K_{D} and active concentration of IL-13 cytokine. The software reports each best fit value along with a 95% confidence interval. Results confirmed that IL13433-1258 and IL413TSLP-1024 bind to cynomolgus monkey IL-13 within 2-fold of the human IL-13 affinity value. IL413P40-0705 had relatively higher affinity for cynomolgus monkey IL-13 compared to the human cytokine (Table 63).

TABLE 63
Affinity of IL13433-1258, IL413TSLP-1024, and IL413P40-0705
Binding to Human or Cynomolgus Monkey IL-13 by KinExA

| | | | Cynomolgus |
|---|---|---|---|
| | | Human IL-13 | Monkey IL-13 |
| Trispecific | | (K_{D}; pM) | (K_{D}; pM) |
| IL13433-1258 | Mean | 0.612 | 0.866 |
| | s.d. | 0.012 | 0.034 |
| IL413TSLP-1024 | Mean | 0.266 | 0.581 |
| | s.d. | 0.022 | 0.020 |
| IL413P40-0705 | Mean | 1.570 | 0.3121 |
| | s.d. | | |

Data shown are mean +/− s.d. using recombinant forms of human IL-13 (R&D Systems), or cynomolgus monkey IL-13 (Pfizer).

### Example 64

### IL-13 Neutralization Activity of Trispecifics IL13433-1258, IL413TSLP-1024, and IL413P40-0705 Compared to Dupilumab

The leading industry antibody aimed at neutralizing the bioactivity of IL-4 and IL-13 in atopic dermatitis is anti-IL-4R dupilumab (Dupixent®; Sanofi/Regeneron) (32), which was the first biologic to be approved for treatment of AD (33). Dupilumab targets the IL-4Rα chain shared by the IL-4 and IL-13 signaling complexes, and neutralizes the activity of both cytokines (34).

We compared the cytokine neutralization activity of the trispecific against dupilumab in the monocyte 0023 expression bioassay. The IL-13 neutralization activity of IIL13433-1258, IL413TSLP-0124, and IL413P40-0705 exceeded that of dupilumab by 7.9×, 7.2×, and 8.9×, respectively (Table 64).

TABLE 64
IL-13 Neutralization Activity of IL13433-1258 and Dupilumab

| | Antibody or | | |
|---|---|---|---|
| Cytokine | Trispecific | | IC₅₀ (pM) ^{a} |
| rHu IL-13 | IL13433-1258 | Median | 11.74 |
| | | s.e.m. | 0.50 |
| | | n | 13 |
| | IL413TSLP- | Median | 12.98 |
| | 1024 | s.e.m. | 1.51 |
| | | n | 7 |
| | IL413P40- | Median | 10.4 |
| | 0705 | s.e.m. | 0.3 |
| | | n | 3 |
| | Dupilumab | Median | 93.2 |
| | | s.e.m. | 6.11 |
| | | n | 6 |

^{a} Mononuclear cells isolated from human peripheral blood were incubated overnight at 37° C. with recombinant human IL-13 (0.25 ng/ml; R&D Systems), along with dilutions of trispecifics or dupilumab. CD23 expression on gated monocytes was quantitated by flow cytometry, and the percentage of CD23-positive cells determined.

### IL413P40-0705 Tri-Fab-Fc Characterization: IL-12/23 Binding Domain

### Example 65

### Bioactivity of Tri-Fab-Fc IL413P40-0705 in Neutralizing IL-12 Mediated STAT4 Phosphorylation and IL-23 Mediated STAT3 Phosphorylation in KIT225 Cells and Whole Blood

The binding of IL-12 to the IL-12 receptor complex, composed of IL-12Rβ1 and IL-12RP2, and the binding of IL-23 to the IL-23 receptor complex, composed of IL-12Rβ1 and IL-23R, lead to receptor complex activation and proximal signaling events that include phosphorylation of STAT4 and STAT3, respectively. IL413P40-0705 was evaluated for its ability to prevent IL12-induced STAT4 phosphorylation or IL23-induced STAT3 phosphorylation in the KIT-225 T-cell line, which is an IL-2 dependent cell line derived from human chronic lymphocytic leukemia peripheral blood. 100 ng/mL (1.7 nM) of IL-12 was used to induce pSTAT4, and 200 ng/mL (3.64 nM) of IL-23 was used to induce pSTAT3, consistent with a pre-determined EC65 value for each stimulus in the assay. Cells were fixed and evaluated by flow cytometry for pSTAT4 or pSTAT3. A comparable version of the assay utilized human whole blood instead of Kit-225 cells, in which 40 ng/mL (0.68 nM) of IL-12 or 150 ng/mL (2.73 nM) of IL-23 were used, consistent with a pre-determined EC65 value for each stimulus in the assay.

Tri-Fab-Fc IL413p40-0705 and p40-0003 (ustekinumab) both neutralized IL-12 and IL-23 in Kit-225 and whole blood assays (Table 65). Because p40-0003 is a bivalent mAb and the Tri-Fab-Fc is monovalent for cytokine binding, the Tri-Fab-Fc is expected to have reduced neutralization capacity on a molar basis. In the Kit-225 assay, IL413p40-0705 was 2.9- and 2.6-fold less potent than p40-0003 at neutralizing IL-12 and IL-23, respectively (Table 65). In the whole blood assay, IL413p40-0705 was 1.5- and 2.4-fold less potent than p40-0003 at neutralizing IL-12 and IL-23, respectively (Table 65).

TABLE 65
Cytokine Neutralization Activity of IL413P40-0705 and p40-0003
(ustekinumab) against IL-12 and IL-23 in Cell-Based Assays

| | | | IC₅₀ (pM) | IC₅₀ (pM) |
|---|---|---|---|---|
| | | | Kit-225 | Whole blood |
| Cyokine | Antibody | | assay^{a} | assay^{b} |
| rHu IL12 | IL413p40-0705 | Mean | 501 | 369 |
| | | s.e.m. | 113 | 60 |
| | | n | 4 | 4 |
| | P40-0003 | Mean | 155 | 262 |
| | (ustekinumab) | s.e.m. | 33 | 33 |
| | | n | 4 | 4 |
| rHu IL23 | IL413p40-0705 | Mean | 2,072 | 9,998 |
| | | s.e.m. | 212 | 726 |
| | | n | 4 | 4 |
| | P40-0003 | Mean | 850 | 4,511 |
| | (ustekinumab) | s.e.m. | 115 | 389 |
| | | n | 4 | 4 |

^{a}Kit-225 cells were stimulated with 100 ng/mL (1.7 nM) of IL-12 or 200 ng/mL (3.64 nM) of IL-23 and analyzed for phospho-STAT4 or phospho-STAT3, respectively, by flow cytometry. IC₅₀ values were calculated based on total mean fluorescent intensity of cell population as function of antibody concentration.
^{b}Human whole blood was stimulated with 40 ng/mL (0.68 nM) of IL-12 or 150 ng/mL (2.73 nM) of IL-23 and analyzed for phospho-STAT4 or phospho-STAT3, respectively, by flow cytometry. IC₅₀ values were calculated based on total mean fluorescent intensity of cell population as function of antibody concentration.

### Example 66

### Neutralization Activity of Tri-Fab-Fc IL413p40-0705 Against Human and Cynomolgus Monkey IL-12 and IL-23

Neutralization of human and cynomolgus monkey IL-12 and IL-23 by IL413p40-0705 were compared using Kit-225 cells. 100 ng/mL (1.7 nM) of IL-12 was used to induce pSTAT4, and 200 ng/mL (3.64 nM) of IL-23 was used to induce pSTAT3, consistent with a pre-determined EC65 value for each stimulus in the assay. Cells were fixed and evaluated by flow cytometry for pSTAT4 or pSTAT3. Both human and cynomolgus monkey recombinant cytokines IL-12 and IL-23 were inhibited by IL413P40-0705, with <2-fold difference in potency between human and cynomolgus monkey for each respective cytokine (Table 66).

TABLE 66
Neutralization Activity of Tri-Fab-Fc IL413p40-
0705 against recombinant Human or Cynomolgus
Monkey IL-12 or IL-23 in Kit-225 assay

| | Kit-225 assay^{a} | | | |
|---|---|---|---|---|
| IC₅₀ | rHuman | rCynomolgus | rHuman | rCynomolgus |
| (pM) | IL-12 | Monkey IL-12 | IL-23 | Monkey IL-23 |
| Median | 501 | 375 | 2,072 | 3,049 |
| s.e.m. | 113 | 53 | 212 | 294 |
| n | 4 | 4 | 4 | 4 |

^{a}Kit-225 cells were stimulated with 100 ng/mL (1.7 nM) of IL-12 or 200 ng/mL (3.64 nM) of human or cynomolgus monkey IL-23 and analyzed for phospho-STAT4 or phospho-STAT3, respectively, by flow cytometry. IC₅₀ values were calculated based on total mean fluorescent intensity of cell population as function of antibody concentration. Recombinant cytokines are designated as rSpecies such as rHuman.

### Example 67

### Comparison of Fab Geometry on Bioactivity of Anti-IL-4/IL-13/p40 Tri-Fab-Fc Variants

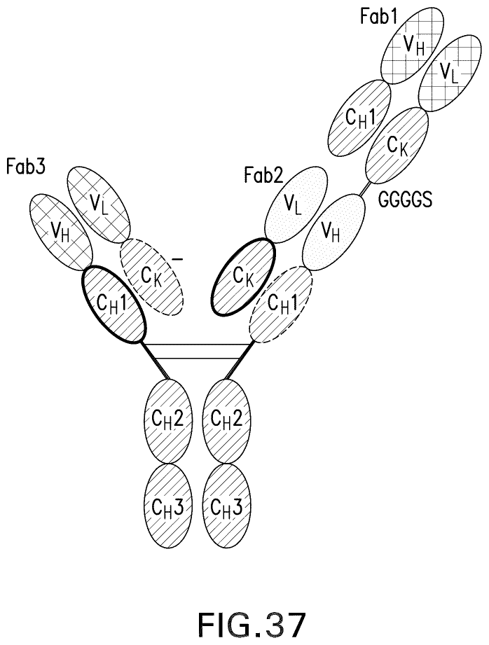

The effect of individual Fab positioning (geometry) within a Tri-Fab-Fc format was characterized by measuring individual target neutralization within various Tri-Fab-Fc constructs (FIG. 37). Neutralization activity of various Tri-Fab-Fc construct were tested in monocyte CD23 expression bioassays for IL-4 (as detailed in Example 4) and IL-13 (as detailed in Example 10) and KIT225 cell line STAT4 phosphorylation bioassays for IL-12 (as detailed in Example 58). Table 67 shows different arrangements of the 3 Fabs in a Tri-Fab-Fc format that were compared. Due to mono-valency of individual Fabs compared to a standard bivalent antibody, IL413p40-0705 was expected to have at least 2-3-fold less bioactivity than respective parental antibody. As it is shown in Table 67, positioning of individual Fabs did not result in loss of activity greater than ˜3 fold, demonstrating that the Fabs maintained bioactivity irrespective of its position within the Tri-Fab-Fc format.

TABLE 67
Individual target neutralization efficacy in different Tri-Fab-Fc constructs

| | | | | IL-4 induced | | IL-13 induced | | IL-12 induced | |
|---|---|---|---|---|---|---|---|---|---|
| | | | | CD23 | | CD23 | | STAT4 | |
| | | | | upregulation | | upregulation | | phosphorylation | |
| Tri-Fab-Fc | Binding domain | | | (IC₅₀; pM) | | (IC₅₀; pM) | | (IC₅₀; nM) | |
| Construct | Fab | Fab | Fab | | parental | | parental | | parental |
| Name | 1 | 2 | 3 | Tri-Fab-Fc | IgG | Tri-Fab-Fc | IgG | Tri-Fab-Fc | IgG |
| IL413p40- | p40- | IL13- | IL4- | 5.10 | 2.06 | 15.1 | 4.7 | 2.61 | 1.77 |
| 0043 | 0003 | 0001 | 0002 | | | | | | |
| IL413p40- | p40- | IL4- | IL13- | 8.11 | 2.06 | 9.14 | 4.7 | 3.03 | 1.77 |
| 0044 | 0003 | 0002 | 0001 | | | | | | |
| IL413p40- | IL13- | IL4- | p40- | 10.3 | 3.23 | 10.4 | 4.38 | 0.50 | 0.155 |
| 0705* | 0001 | 1040 | 0003 | | | | | | |

*Neutralization IC50 of parental IgG tested with IL413p40-0705 is different due to values being generated from a separate experiment.

### Example 68

### Kinetic Evaluation of Anti-IL-4/IL-13/p40 Tri-Fab-Fc IL413p40-0705 Binding Human and Cynomolgus Monkey IL-12 and IL-23 Using Surface Plasmon Resonance

Surface plasmon resonance (SPR) was performed to determine the affinity constants for Tri-Fab-Fc against IL-12 and IL-23 that contain p40 binding domain. For these analyses, kinetic assays were conducted at 37° C. at a collection rate of 10 Hz on a BIAcore™ 8K instrument (GE Healthcare). Anti-human IgG antibody (catalog number BR-1008-39, GE Healthcare) was amine coupled to all four flow cells of a carboxymethylated dextran coated sensor chip (CM5) (GE Healthcare) using the manufacturer's protocol and IL413p40-0705 was immobilized to ˜10 RU. Next, various concentrations of human IL-12, cyno IL-12, human IL-23 or cyno IL-23 was injected over the surface. Table 68 shows measured affinity constants of IL413p40-0705 against human and cyno IL-12 and IL-23.

The anti-IL-4 and anti-IL13 binding domains exhibit extremely slow off-rates (kd) to their cognate target which made it difficult to accurately define affinity constants since they were at the limit of detection. Therefore, KinExA™ methodology was employed to accurately determine affinity for the Tri-Fab-Fc to human IL-4 and IL-13 (Example 25).

TABLE 68
IL413p40-0705 Affinity Constants Obtained using
Surface Plasmon Resonance against recombinant
Human or Cynomolgus Monkey IL-12 or IL-23

| | rHuman | rCyno | rHuman | rCyno |
|---|---|---|---|---|
| Tri-Fab-Fc | IL-12 | IL-12 | IL-23 | IL-23 |
| Affinity | 114.32 ± | 253.79 ± | 91.5 ± | 235.94 ± |
| (pM) | 4.87 | 6.84 | 4.61 | 8.62 |

### Example 69

### Kinetic Evaluation of IL413p40-0705 Tri-Fab-Fc Binding to Recombinant Human and Cynomolgus IL-4 and IL-13 Cytokines using KinExA™ Methodology

A Kinetics Exclusion Assay (KinExA™) instrument (model 3200, Sapidyne) was used to accurately determine the binding affinity of the anti-IL4/IL-13/p40 Tri-Fab-Fc to recombinant human IL-4 and IL-13. Data analysis was performed with the KinExA™ Pro software version 3.6.5 (Sapidyne). The “affinity standard” model was used to analyze the data, determine the apparent K_{D} and apparent active concentration of the recombinant human and cynomolgus monkey IL-13, and human and cynomolgus monkey IL-4. The “drift correction” was used when appropriate. Multiple curves were obtained, both receptor and K_{D} controlled, from independent experiments and analyzed using the “n-curve analysis” tool to obtain global best fit values for the K_{D} and active concentration. The software reports each best fit value along with a 95% confidence interval. Results showed that Tri-Fab-Fc IL413p40-0705 binds to human IL-4 and IL-13 with 739 fM and 1.57 pM affinity, respectively and to cynomolgus IL-4 and IL-13 with 1.57 pM and 312.1 fM, respectively (Table 69).

TABLE 69
IL413p40-0705 Affinity Constants Obtained using the
Kinetics Exclusion Assay (KinExA ™)

| Tri-Fab-Fc | Human IL-4 | Cyno IL-4 | Human IL-13 | Cyno IL-13 |
|---|---|---|---|---|
| Affinity (fM) | 739.0 | 918.0 | 1570 | 312.1 |

### Example 70

### Species Specificity of IL413p40-0705 Tri-Fab-Fc by Surface Plasmon Resonance

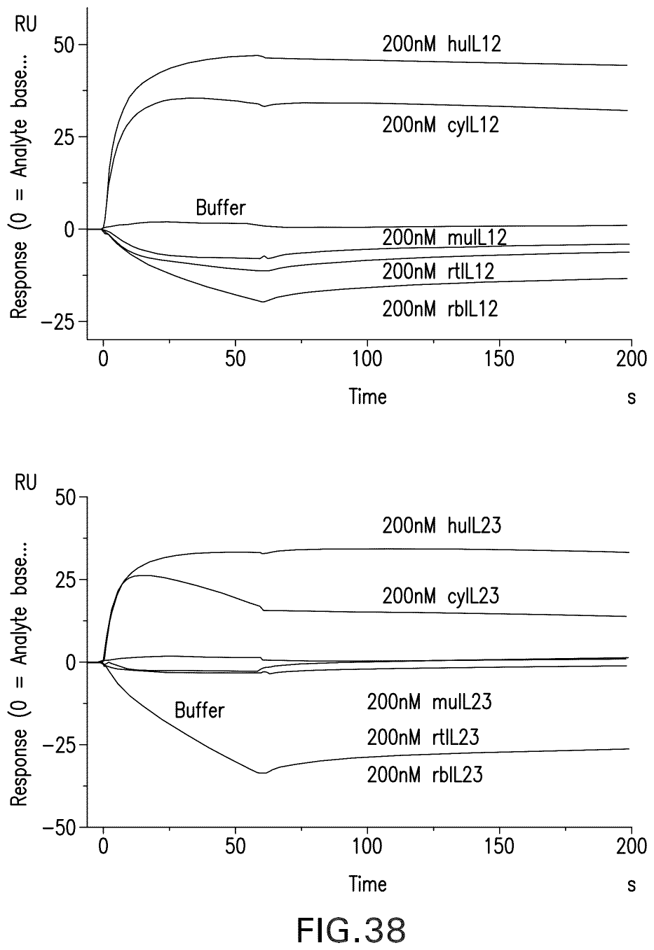

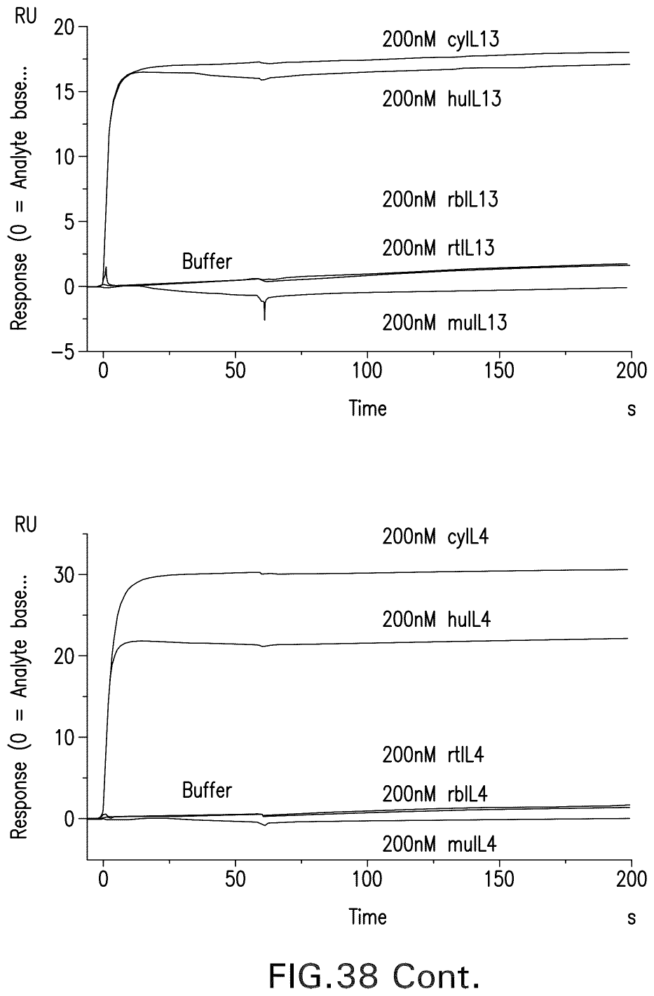

To evaluate the species specificity of Tri-Fab-Fc in addition to human and cynomolgus monkey, mouse, rat and rabbit IL-12, IL-23, IL-13 and IL-4 were tested by surface plasmon resonance. For these analyses, kinetic assays were conducted at 37° C. at a collection rate of 10 Hz on a BIAcore™ 8K instrument (GE Healthcare). Anti-human IgG antibody (catalog number BR-1008-39, GE Healthcare) was amine coupled to all four flow cells of a carboxymethylated dextran coated sensor chip (CM5) (GE Healthcare) using the manufacturer's protocol and the Tri-Fab-Fc IL413p40-0705 was captured at RU ˜100. Next, 200 nM of the human, cynomolgus monkey, rat, rabbit or mouse IL-12, IL-23, IL-13 and IL-4 were injected over the surface. As it is shown in the sensorgram (FIG. 38), IL413p40-0705 binds to human and cynomolgus monkey cytokines IL-12, IL-23, IL-4, and IL-13 but not to mouse, rat or rabbit homologs.

### Example 71

### Bioactivity of Anti-p40 Antibody p40-0003 in Neutralizing IL-12 Mediated STAT4 Phosphorylation and IL-23 Mediated STAT3 Phosphorylation in KIT225 Cells and Whole Blood

The binding of IL-12 to the IL-12 receptor complex, comprised of IL-12Rβ1 and IL-12Rβ2, and IL-23 to the IL-23 receptor complex, comprised of IL-12Rβ1 and IL-23R, lead to receptor complex activation and proximal signaling events that include phosphorylation of STAT4 and STAT3, respectively. P40-0003 was evaluated for its ability to prevent IL12-induced STAT4 phosphorylation or IL23-induced STAT3 phosphorylation in the KIT-225 T-cell line, which is an IL-2 dependent cell line derived from human chronic lymphocytic leukemia peripheral blood. 100 ng/mL (1.7 nM) of IL-12 was used to induce pSTAT4, and 200 ng/mL (3.64 nM) of IL-23 was used to induce pSTAT3, consistent with a pre-determined EC65 value for each stimulus in the assay. Cells were fixed and evaluated by flow cytometry for pSTAT4 or pSTAT3. A comparable version of the assay utilized human whole blood instead of Kit-225 cells, in which 40 ng/mL (0.68 nM) of IL-12 or 150 ng/mL (2.73 nM) of IL-23 were used, consistent with a pre-determined EC65 value for each stimulus in the assay. Anti-p40 antibody p40-0003 neutralizes IL-12 mediated STAT4 phosphorylation and IL-23 mediated STAT3 phosphorylation in KIT225 cells and whole blood (Table 70).

TABLE 70

| | | | IC₅₀ (pM) | IC₅₀ (pM) |
|---|---|---|---|---|
| | | | Kit-225 | Whole blood |
| Cyokine | Antibody | | assay^{a} | assay^{b} |
| rHu IL12 | P40-0003 | Mean | 155 | 262 |
| | | s.e.m. | 33 | 33 |
| | | n | 4 | 4 |
| rHu IL23 | P40-0003 | Mean | 850 | 4,511 |
| | | | 115 | 389 |
| | | s.e.m. | 4 | 4 |
| | | n | | |

^{a}Kit-225 cells were stimulated with 100 ng/mL (1.7 nM) of IL-12 or 200 ng/mL (3.64 nM) of IL-23 and analyzed for phospho-STAT4 or phospho-STAT3, respectively, by flow cytometry. IC₅₀ values were calculated based on total mean fluorescent intensity of cell population as function of antibody concentration.
^{b}Human whole blood was stimulated with 40 ng/mL (0.68 nM) of IL-12 or 150 ng/mL (2.73 nM) of IL-23 and analyzed for phospho-STAT4 or phospho-STAT3, respectively, by flow cytometry. IC₅₀ values were calculated based on total mean fluorescent intensity of cell population as function of antibody concentration.

### IL33433-1258 Tri-Fab-Fc Characterization: IL-33 Binding Domain

### Example 72

### IL-33 Neutralization Activity of IL33-158-152, IL33-158LS, IL33-0726, and IL13433-1258 in a Reporter Cell Assay

IgG antibody clone IL33-158-152 was the template variant used for affinity and deamidation liability removal engineering efforts. IL33-158LS was the extended half-life variant. Clone IL33-0726 was the affinity-biophysical property improved IgG antibody. The IL33-0726 binding domain (Fab) was incorporated into trispecific IL13433-1258.

IL-33 binding to the IL-33 receptor complex comprised of IL1RL1 (also known as ST2) and IL1RAP results in an intracellular signaling cascade activating the MyD88/NF-kB and MAPK/AP-1 signaling pathways (37). HEK-Blue™ IL-33 Cells (Invivogen) are a HEK293-based cell line engineered to lack TNF and IL-1 signaling and stably express both IL1 RL1 and a NF-κB/AP-1-inducible secreted embryonic alkaline phosphatase (SEAP) reporter gene. Upon IL-33 stimulation, these cells secrete SEAP, which can subsequently be quantitated using a colorimetric assay to assess activity of IL-33, as described in EXAMPLE 46. Trispecific IL13433-1258, mAb IL33-158-152, the template variant used for affinity engineering efforts, extended half-life variant IL33-158LS, and the affinity-improved IgG form IL33-0726 were all able to inhibit IL-33 activity as evidenced by the ability to suppress SEAP activity in HEK-Blue™ IL-33 Cells. However, IL13433-1258 demonstrated superior neutralizing activity compared to the template used for affinity engineering efforts, as evidenced by the lower IC₅₀ values of 13.40 pM compared to 42.85 pM for IL33-158-152 and 56.22 pM for IL33-158LS (Table 71). The affinity-improved IgG clone IL33-0726 showed slightly lower IC₅₀ values compared to IL13433-1258; however, it is important to note that IL33-0726 has two IL-33 binding epitopes in IgG format compared to the single binding epitope found in the trispecific format of IL13433-1258.

### Example 73

### IL-33 Neutralization Activity of IL33-158LS and IL13433-1258 in a Primary Cell Assay

IL-33 has also been reported to synergize with IL-12 to induce the expression of IFNγ from T lymphocytes (38, 39). To assess their IL-33 neutralization activity, clones were assayed for the ability to inhibit this synergy with IL-12 to produce IFNγ. Briefly, 175 μL of heparinized whole blood from healthy donors was pipetted into 96-well v-bottom plates. Recombinant human IL-12 (R&D Systems 219-IL) was diluted to 40 ng/mL in RPMI 1640 (Gibco 21870-078) supplemented with 1× pen/strep/glu (Invitrogen 10378-016) and 10% heat-inactivated FBS (Gibco 16140-171). 5 μL of the 40 ng/mL IL-12 was added to each of the wells containing the heparinized blood and was incubated in a 37° C. incubator with 5% CO₂, for 3 hours. At this time, 10 μL of the test antibody dilutions was added to the 180 μL of cells/IL-12 in each well followed by 10 μL of purified IL-33-mm2 (Pfizer) to achieve a final IL-33 concentration of 0.125 nM. The IL-33-mm2 variant is a human IL-33 variant in which all four cysteine residues within IL-33 are mutated to serines in order to prevent redox-induced degradation of IL-33 that would inhibit signaling and assay windows in whole blood. Cells were then incubated for approximately 22 hours in a 37° C. incubator with 5% CO₂. At this time, the plasma was separated from the blood by centrifugation, and IFNγ levels were quantitated by ELISA (Meso Scale Discovery L151AEB-2). Antibody activity was assessed by the inhibition of IL-33/IL-12-induced IFNγ. IL13433-1258, clone IL33-158-152, the template variant used for affinity and deamidation liability removal engineering efforts, extended half-life variant IL33-158LS, and the affinity-improved IgG form IL33-0726 were all able to inhibit IL-33 activity as evidenced by the reduction in IFNg release from T lymphocytes following stimulation with IL-33 and IL-12. Compared to IL33-158-152 and IL33-158LS, trispecific IL13433-1258 demonstrated superior IL-33 neutralizing activity (Table 71).

TABLE 71
Cytokine Neutralization Activity against
IL-33 in Cell-Based Assays

| | | | IC₅₀ (pM) | IC₅₀ (pM) |
|---|---|---|---|---|
| | Antibody or | | HEK-Blue ™ | IFNg Whole |
| Cytokine | Trispecific | | IL-33 SEAP^{a} | Blood^{b} |
| rHu IL-33 | IL33-158LS | Mean | 56.22 | |
| | | s.e.m. | 6.05 | |
| | | N | 6 | |
| | IL33-158-152 | Mean | 42.85 | |
| | | s.e.m. | 8.10 | |
| | | N | 8 | |
| | IL33-0726 | Mean | 8.27 | |
| | | s.e.m. | 1.57 | |
| | | N | 7 | |
| | IL13433-1258 | Mean | 13.40 | |
| | | s.e.m. | 2.61 | |
| | | n | 16 | |
| IL-33 MM2- | IL33-158LS | Mean | | 16.20 |
| Cys (0.125 | | s.e.m. | | 4.03 |
| nM) | | n | | 5 |
| | IL13433-1258 | Mean | | 12.48 |
| | | s.e.m. | | 1.74 |
| | | n | | 9 |

^{a}SEAP activity was assayed in HEK-Blue ™ cells exposed to IL-33 (0.1 ng/mL) for 20 hours at 37° C.
^{b}IFNg levels were measured in the supernatant obtained from heparinized human whole blood stimulated with IL-12 (1 ng/mL) and IL-33 mm2-Cys mutant (0.125 nM ) for 22 hours at 37° C.

### Example 74

### Species Specificity for IL-33 in IL13433-1258 in Cell Reporter Assays

Human and cynomolgus monkey IL-33 were also compared using HEK-Blue™ IL-33 Cells (Invivogen), as described in EXAMPLE 72. In agreement with SPR data, both human and cynomolgus monkey IL-33 are inhibited by IL13433-1258; however, cynomolgus monkey IL-33 binds to IL13433-1258 more weakly than human IL-33 (Table 72).

TABLE 72
Neutralization Activity of Trispecific IL13433-
1258 against Human or Cynomolgus Monkey IL-33

| | Human IL-33 | Cynomolgus Monkey IL-33 |
|---|---|---|
| IL-33 | (IC₅₀; pM) | (IC₅₀; pM) |
| Median | 13.40 | 274.4 |
| s.e.m. | 2.61 | 30.62 |
| n | 16 | 10 |

Data shown are median and s.e.m. of IC₅₀ (pM) from the indicated number of individual bioassay experiments. IL-33 neutralization activity was determined in the SEAP activity assay in HEK-Blue ™ IL-33 SEAP cells exposed to IL-33 (0.1 ng/mL) for 20 hours at 37° C., using recombinant forms of human IL-33 (R&D Systems), or cynomolgus monkey IL-33 (Pfizer).

### Example 75

### Species Specificity for IL-33 in IL13433-1258 Using Surface Plasmon Resonance

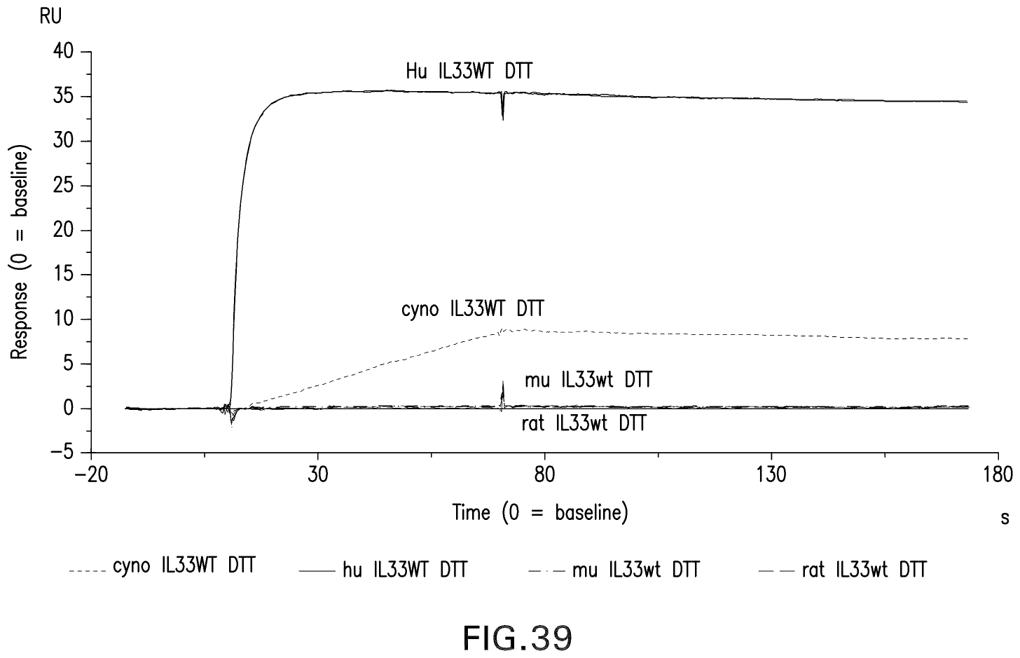

Surface plasmon resonance (SPR) with a Biacore T-200 was used to evaluate the cross-species specificity of trispecific IL13433-1258 (Pfizer, 00705757-0296) to human, cynomolgus monkey, mouse, and rat IL-33. Experiments were done at 37° C. with a collection rate of 10 Hz using HBS-EP+ pH 7.4, containing 3 mM DTT as running and dilution buffer. Human IL-33 wild type (WT) (Pfizer, 42564-166), murine IL-33 WT (R&D 3626-101/CF) and rat IL-33 WT (Creative Biomart IL33-583R) were reduced in 3 mM DTT (Pierce No-Weigh, 20291) for 2 hours at room temperature. Cynomolgus monkey IL-33 WT (Pfizer, WRS-072216) was provided in PBS containing 1 mM DTT. Briefly, Protein A/G (Pierce, 21186) was immobilized on all 4 flow cells of a CM5 sensor chip (GE Healthcare, Marlborough, MA 29-1496-03) using an amine coupling kit (GE Healthcare, Marlborough, MA BR100050) according to the manufacturer's protocol. IL13433-1258 was captured on flow cells 3 and 4 at a concentration of 1.5 μg/mL by injecting for 30 sec with a flow rate of 10 μL/min. An anti-rat and mouse positive control antibody M36 (Pfizer, L4295010-051) diluted to 10 μgs/mL was captured on flow cell 2 for 60 sec at 10 μLs/min. Flow cell 1 was used as a reference. Binding was evaluated by injecting 200 nM dilutions of each cytokine over all 4 flow cells for 60 sec at 50 μL/min followed by a 300 sec dissociation. The surface was regenerated with a single 60 sec injection of 10 mM Glycine pH 1.5 at 50 μL/min. The data was double referenced and an overlay of the resulting sensorgrams (FIG. 39) was prepared using Biacore T-200 Analysis Software version 3.2. Human and cynomolgus monkey IL-33 bind to IL13433-1258; however, murine and rat IL-33 do not demonstrate observable binding at 200 nM. Cynomolgus monkey IL-33 binding to IL13433-1258 is weaker than human IL-33. Murine and rat IL-33 did bind to the rat and mouse specific control antibody M36 as expected.

SPR was used to evaluate simultaneous binding of IL13433-1258 to all three target cytokines, and confirmed that IL-4, IL-13, and IL-33 could simultaneously bind to the IL13433-1258 trispecific regardless of the injection order (EXAMPLE 40).

### Example 76

### Binding Affinity of IL13433-1258 to Human and Cynomolgus Monkey IL-33 by KinExA

A Kinetic exclusion assay (KinExA) instrument (model 3200, Sapidyne) was used to determine the binding affinity of trispecific IL13433-1258 to human IL-33 and cynomolgus monkey IL-33. Samples were prepared in PBS containing 0.1% sodium azide and 1.0 mg/ml BSA. The fixed antigen assay method was used to determine binding affinity. Trispecific IL13433-1258 was serially diluted 2-fold from 2 nM to 31fM and titrated with biotinylated human IL-33 (Pfizer) or biotinylated cynomolgus monkey IL-33 (Pfizer) with concentrations that were kept constant at 5 pM and 100 pM for human IL-33 and cynomolgus monkey IL-33. Biotinylated human IL-33 was reduced with 3 mM DTT for 2 hours under room temperature before use. Trispecific IL13433-1258 and IL-33 cytokine were equilibrated for at least 72 hours at room temperature, then passed through a flow cell containing Polymethylmethacrylate (PMMA) beads coated with anti-hIL-33 Antibody 0726 (Pfizer) that contains the same IL-33 binding domain as trispecific IL13433-1258. The free IL-33 captured with anti-hIL-33 Antibody-0726 was detected with 0.5 ug/ml Alexa Fluor 647-conjugated streptavidin (Jackson Immunoresearch). Data analysis was performed with KinExA Pro software version 4.3.11 (Sapidyne). The ‘affinity standard’ model was used to analyze the data and determine the K_{D} and active concentration of the IL-33 cytokine. The ‘drift correction’ fitting option was used when responses varied between replicate injections. Two curves were obtained in independent experiments and analyzed using the ‘n-curve analysis’ tool to obtain global best fit values for the K_{D} and active concentration of IL-33 cytokine. The software reports each best fit value along with a 95% confidence interval. Results confirmed that IL13433-1258 binds to cynomolgus monkey IL-33 about 280-fold weaker compared to human IL-33 affinity value (FIG. 39, Table 73).

TABLE 73
Affinity of IL13433-1258 Binding to Human
or Cynomolgus Monkey IL-33 by KinExA

| | Human IL-33 | Cynomolgus Monkey IL-33 |
|---|---|---|
| IL-33 | (K_{D}; pM) | (K_{D}; pM) |
| Mean | 0.151 | 43.30 |
| s.d. | 0.0016 | 0.580 |

Data shown are mean +/− s.d. using recombinant forms of human IL-33 (Pfizer), or cynomolgus monkey IL-33 (Pfizer).

### Example 77

### IL-33 Neutralization Activity of Trispecific IL13433-1258 Compared to Known IL-33 Antibodies

Several antibodies are known to neutralize the bioactivity of IL-33, including IL33-265, IL33-310, IL33-301, and IL33-303. IL33-265 is an antibody with the variable regions of the IL-33 antibody Itepekimab (REGN3500; Regeneron Pharmaceuticals, Inc.: see SEQ ID NO: 274 and 282 of US2014/0271658) grafted to human IgG1 effector function null constant regions in place of the original human IgG4. IL33-265 is identical in sequence to IL33-0352 except that IL33-265 encodes an additional residue at the C terminus of the heavy chain. This residue, a lysine, is normally cleaved from the protein during expression in mammalian cells, so the resulting IL33-265 and IL33-0352 are identical in protein sequence. IL33-310 is composed of SEQ ID NO: 306 (HC), SEQ ID NO: 307 (LC) from US2016168242 (Genentech, Inc.) IL33-301 and IL33-303 are derived from WO2015/106080 (AnaptysBio). GBT-IL33-0301 contains SEQ ID NO: 124 (VH) paired with SEQ ID NO: 142 (VL), while GBT-IL33-0303 contained the same VH paired with SEQ ID NO: 173 (VL). IL33-0301 is closely related to Etokimab (AnaptysBio, Inc.), differing by two amino acids in the VH (V5M in FW1, D56N in CDRH2; Kabat numbering ) and two in the VL (Q92K S93T in CDRL3). Furthermore, IL33-0301 and IL33-0303 use human IgG1 constant region with mutations to minimize effector function, while Etokimab uses a wild-type human IgG1. A series of experiments was run to compare the activity of these known antibodies with IL13433-1258.

Briefly, human IL-33 was used to assess the neutralizing activity of IL13433-1258 and known IL-33 antibodies using HEK-Blue™ IL-33 Cells (Invivogen), as described in EXAMPLE 72. IL13433-1258, 1L33-265, IL33-301, IL33-303, and IL33-310 were all able to inhibit IL-33 activity as evidenced by the ability to suppress SEAP activity in HEK-Blue™ IL-33 Cells. However, IL13433-1258 demonstrated superior neutralizing activity compared to IL33-301, IL33-303, and IL33-310, while having comparable activity to that of IL33-265 (Table 74).

TABLE 74
Cytokine Neutralization Activity of IL13433-1258
and known IL-33 antibodies in Cell-Based Assays

| | Antibody or | | IC₅₀ (pM) HEK- |
|---|---|---|---|
| Cytokine | Trispecific | | Blue ™ IL-33 SEAP^{a} |
| rHu IL-33 | IL33-301 | Mean | 596.5 |
| | | s.e.m. | 149.5 |
| | | n | 13 |
| | IL-33-303 | Mean | 769.1 |
| | | s.e.m. | 186.0 |
| | | n | 7 |
| | IL33-310 | Mean | 56.40 |
| | | s.e.m. | 8.99 |
| | | n | 6 |
| | IL33-265 | Mean | 15.91 |
| | | s.e.m. | 1.31 |
| | | n | 12 |
| | IL13433-1258 | Mean | 13.40 |
| | | s.e.m. | 2.61 |
| | | n | 16 |

^{a}SEAP activity was assayed in HEK-Blue ™ cells exposed to IL-33 (0.1 ng/mL) for 20 hours at 37° C.

### IL413TSLP-1024, IL413TSLP-1028 and IL413TSLP-1037 Tri-Fab-Fcs Characterization: TSLP Binding Domain

### Example 78

### TSLP Neutralization Activity of mAb TSLP-0001, mAb TSLP-0875, mAb TSLP-0855, mAb TSLP-0871, and Trispecific IL413TSLP-1024, IL413TSLP-1028 and IL413TSLP-1037

The innate cytokine alarmin TSLP is elevated in AD and asthma and has been implicated in promoting type 2 immune responses at the barrier surfaces (40-42). TSLP is produced by epithelial cells, keratinocytes, and fibroblasts. TSLP binds to a heterodimeric receptor comprised of TSLPR and IL-7Rα on a range of immune cell types, and promotes epithelial cross-talk, resulting in activation of DCs, production of type 2 cytokines, and activation of Th2 effector responses (4, 43).

TSLP-0001 was the template variant used for affinity engineering efforts. TSLP-0855, TSLP-0871 and TSLP-0875, were the affinity-improved mAb clones. TSLP neutralization activity of TSLP-0001, TSLP-0855, TSLP-0871, TSLP-0875, and trispecifics IL413TSLP-1024, IL413TSLP-1028 and IL413TSLP-1037 was evaluated in a cell-based bioassay. The TSLP bioassay examines release of the chemokine TARC by stimulated monocytes, as described in EXAMPLE 19. Primary human monocytes isolated from peripheral blood were incubated overnight at 37° C. with 0.5 ng/ml glycosylated recombinant human TSLP (Pfizer BMD), along with dilutions of the antibodies or trispecifics. Supernatants were harvested and assayed for TARC by MSD.

Anti-TSLP clone TSLP-0001 (Tezepelumab; AZ/Amgen) inhibited TSLP bioactivity in the monocyte TARC production assay (Table 75). Affinity-improved TSLP-binding domain TSLP-0875, TSLP-0855 and TSLP-0871 were derived from clone TSLP-0001 and was incorporated into trispecific IL413TSLP-1024, IL413TSLP-1028 and IL413TSLP-1037 respectively. Table 75 compares the TSLP neutralization activity for trispecific IL413TSLP-1024, IL413TSLP-1028 and IL413TSLP-1037 compared with constituent TSLP binding domain, TSLP-0875, TSLP-0855 and TSLP-0871 in mAb format. Because mAb is bivalent and the trispecific is monovalent for cytokine binding, the trispecific is expected to have reduced neutralization capacity on a molar basis. For TSLP, the trispecifics had ˜ 2.5× lower potency than mAbs (Table 75).

TABLE 75
TSLP Neutralization Activity of mAb TSLP-0001, mAb
TSLP-0875, TSLP-0855 and TSLP-0871, and Trispecific
IL413TSLP-1024, IL413TSLP-1028 and IL413TSLP-1037

| | Antibody or | | |
|---|---|---|---|
| Cytokine | Trispecific | | IC₅₀ (pM) |
| rHu TSLP | TSLP-0001 | Median | 19.15 |
| | | s.e.m. | 1.42 |
| | | n | 30 |
| | TSLP-0875 | Median | 5.56 |
| | | s.e.m. | 0.95 |
| | | n | 4 |
| | TSLP-0855 | Median | 4.12 |
| | | s.e.m. | 0.66 |
| | | n | 2 |
| | TSLP-0871 | Median | 5.69 |
| | | s.e.m. | 0.89 |
| | | n | 2 |
| | IL413TSLP-1024 | Median | 13.99 |
| | | s.e.m. | 1.49 |
| | | n | 11 |
| | IL413TSLP-1028 | Median | 12.5 |
| | | s.e.m. | 7.5 |
| | | n | 2 |
| | IL413TSLP-1037 | Median | 18.5 |
| | | s.e.m. | 4.5 |
| | | n | 2 |

TARC production was assayed in human peripheral blood monocytes or human whole blood exposed to TARC (4 ng/ml) for 24 hours at 37° C.

### Example 79

### Reactivity of IL413TSLP-1024 Against Short Form and Long Form TSLP

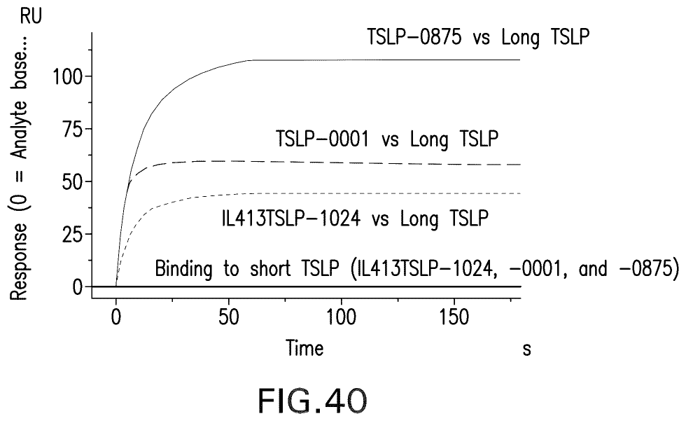

TSLP exists in a short form, maintained under homeostatic conditions, and a long form, induced with inflammation (44). Functions of the short form, and its cell surface receptor, have not been well characterized, but antimicrobial activity has been proposed (45). Structural analysis indicates that the long form is targeted by tezepelumab, which likely does not form contacts with the sequence of the short form (46). Because binding domain TSLP-0875 was derived from tezepelumab (TSLP-0001), it is unlikely to interact with the short form. To confirm the binding specificity, both short form and long form TSLP constructs were generated and protein produced. SPR was used to confirm the binding specificity of trispecific IL413TSLP-1024 and anti-TSLP antibodies TSLP-0001 and TSLP-0875 to TSLP short form and long form proteins. High concentrations (300 nM) of short form or long form TSLP were injected over anti-Fab captured IL413TSLP-1024, TSLP-0001 and TSLP-0875, with binding detected by surface plasmon resonance, as described in EXAMPLE 52. SPR analysis confirmed that TSLP-0001, TSLP-0875, and IL413TSLP-1024 did not bind to the short form (FIG. 40). TSLP-0001, TSLP-0875, or IL413TSLP-1024 was captured by anti-Fab antibody (GE HealthCare, Marlborough, MA) which was immobilized on Biacore CM5 sensor chip. Short form (TSLPsf Avi V5 His 10 Biotin) or long form (TSLPlf Avi V5 His 10 Biotin) TSLP isomers, at a concentration of 300 nM, were injected over the captured trispecifics/mAbs to test binding. This analysis was done using a Biacore 8K at 37° C. TSLP-0001, TSLP-0875, or IL413TSLP-1024 do not show any binding to the short form of TSLP as evaluated by SPR. As expected, TSLP-0001, TSLP-0875, or IL413TSLP-1024 bind the long form TSLP.

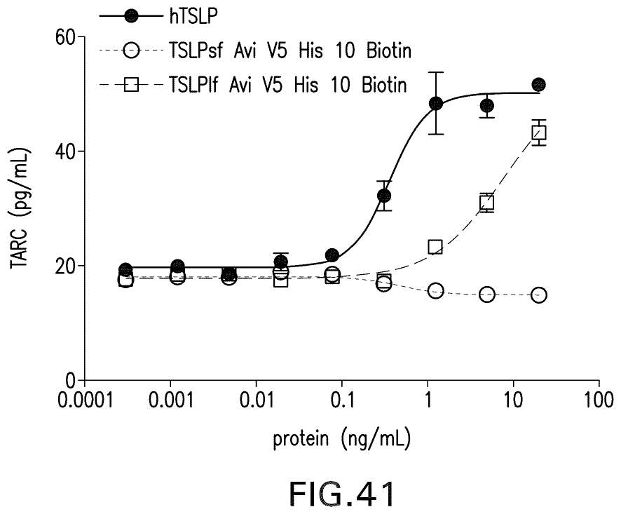

Similarly, the short form did not have activity in the monocyte TARC production bioassay (FIG. 41). These findings confirm that IL413TSLP-1024 has selective reactivity against the long form of TSLP.

### Example 80

### Neutralization Activity of IL413TSLP-1024 Against Human and Cynomolgus Monkey TSLP in Monocytes and Whole Blood

To confirm antibody activity against cynomolgus monkey cytokines, neutralization activity of IL413TSLP-1024 was tested in the monocyte TARC production bioassay. Bioactivity of human or cynomolgus monkey TSLP (Pfizer) was efficiently inhibited by trispecific IL413TSLP-1024. Neutralization activity against cynomolgus TSLP was ˜2.1× reduced compared to the human cytokine (Table 76). Experiments in the monocyte TARC production assay adapted to whole blood format confirmed that the trispecific IL413TSLP-1024 blocks cytokine activity in human and cynomolgus monkey whole blood, in addition to isolated monocytes, (Table 76).

TABLE 76
Neutralization Activity of Trispecific IL413TSLP-
1024 against Human or Cynomolgus Monkey TSLP

| | Peripheral Blood Monocytes | | Human Whole Blood | |
|---|---|---|---|---|
| | (IC₅₀; pM) | | (IC₅₀; pM) | |
| | | Cynomolgus | | Cynomolgus |
| TSLP | Human | Monkey | Human | Monkey |
| Median | 13.99 | 30.00 | 8.44 | 32.67 |
| s.e.m. | 1.49 | 2.02 | 2.29 | 9.85 |
| n | 11 | 7 | 6 | 6 |

TSLP neutralization activity was assayed using the monocyte TARC production bioassay, using recombinant forms of human TSLP (Pfizer), or cynomolgus monkey TSLP (Pfizer). TARC production was assayed in human peripheral blood monocytes or human whole blood exposed to TARC (4 ng/ml) for 24 hours at 37° C.

### Example 81

### Binding of IL413TSLP-1024 to Human, Cynomolgus Monkey, Mouse, and Rat TSLP by Surface Plasmon Resonance

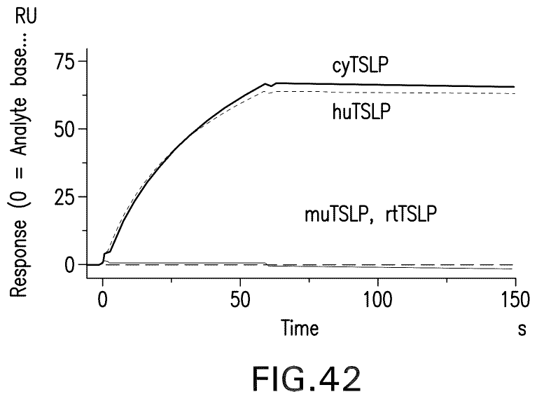

Cross-species studies using surface plasmon resonance (SPR) characterized the binding of IL413TSLP-1024 to human, cynomolgus monkey, mouse, and rat TSLP. All experiments were performed using Biacore 8K instrument (GE HealthCare, Marlborough, MA). A Biotin CAP Kit (GE HealthCare, Marlborough, MA) was used to capture biotinylated human, cynomolgus monkey, mouse, and rat TSLP following the instructions provided by the manufacturer (GE HealthCare, Marlborough, MA). Trispecific IL413TSLP-1024, or control anti-mouse TSLP antibody (AF555; R&D Systems) was flowed over the captured biotinylated TSLP at a concentration of 300 nM. The association and dissociation phases were 60 seconds and 300 seconds, respectively. At the end of the dissociation phase, the surface containing streptavidin was regenerated using one 120 second pulse of freshly prepared 3:1 8M guanidine-HCl:1M NaOH. FIG. 42 shows that IL413TSLP-1024 binds only to human and cynomolgus monkey TSLP at a concentration of 300 nM.

### Example 82

### Binding Affinity of IL413TSLP-1024 to Human and Cynomolgus Monkey TSLP by KinExA

A Kinetics exclusion assay (KinExA) instrument (model 3200, Sapidyne) was used to determine the binding affinity of trispecific IL413TSLP-1024 to human and cynomolgus monkey TSLP. Samples were prepared in PBS containing 0.1% sodium azide and 1.0 mg/ml BSA. A fixed antigen assay method was used to determine binding affinity. Trispecific IL413TSLP-1024 was serially diluted 2-fold from 400 pM to 12 fM and titrated with biotinylated TSLP from human (Pfizer) and cynomolgus monkey (Pfizer) with concentrations that were kept constant at either 10 pM or 100 pM. Trispecific IL413TSLP-1024 and TSLP cytokine were equilibrated for at least 72 hours at room temperature, then passed through a flow cell containing Polymethylmethacrylate (PMMA) beads coated with anti-TSLP mAb TSLP-0875 (Pfizer) that contains the same TSLP binding domain as trispecific IL413TSLP-1024. Bound biotinylated TSLP was detected with 1 ug/ml Alexa Fluor 647-Streptavidin (Jackson Immunoresearch). Data analysis was performed with the KinExA Pro software version 4.3.11 (Sapidyne). The affinity standard model was used to analyze the data and determine the K_{D} and active concentration of the biotinylated TSLP antigen. The ‘drift correction’ option was selected when responses varied between replicate injections. Two curves were obtained in independent experiments and analyzed using the ‘n-curve analysis’ tool to obtain global best fit values for the K_{D} and active concentration of biotinylated TSLP. The software reports each best fit value along with a 95% confidence interval). Results confirmed that IL413TSLP-1024 binds to cynomolgus monkey TSLP within about 2-fold of the human TSLP affinity value. (FIG. 8, Table 77).

TABLE 77
Affinity of IL413TSLP-1024 Binding to Human
or Cynomolgus Monkey TSLP by KinExA

| | Human TSLP | Cynomolgus Monkey TSLP |
|---|---|---|
| | (K_{D}; pM) | (K_{D}; pM) |
| Mean | 3.03 | 6.31 |
| % Error | 2.6% | 1.7% |

Data shown are mean and % Error. using recombinant forms of human TSLP (Pfizer), or cynomolgus monkey TSLP (Pfizer). % Error is a curve fit quality measurement and resulted from a quantitative comparison between the best fit theory curve and the measured data points.

### Example 83

### Simultaneous Binding of IL-4, IL-13, and TSLP to IL413TSLP, by Surface Plasmon Resonance

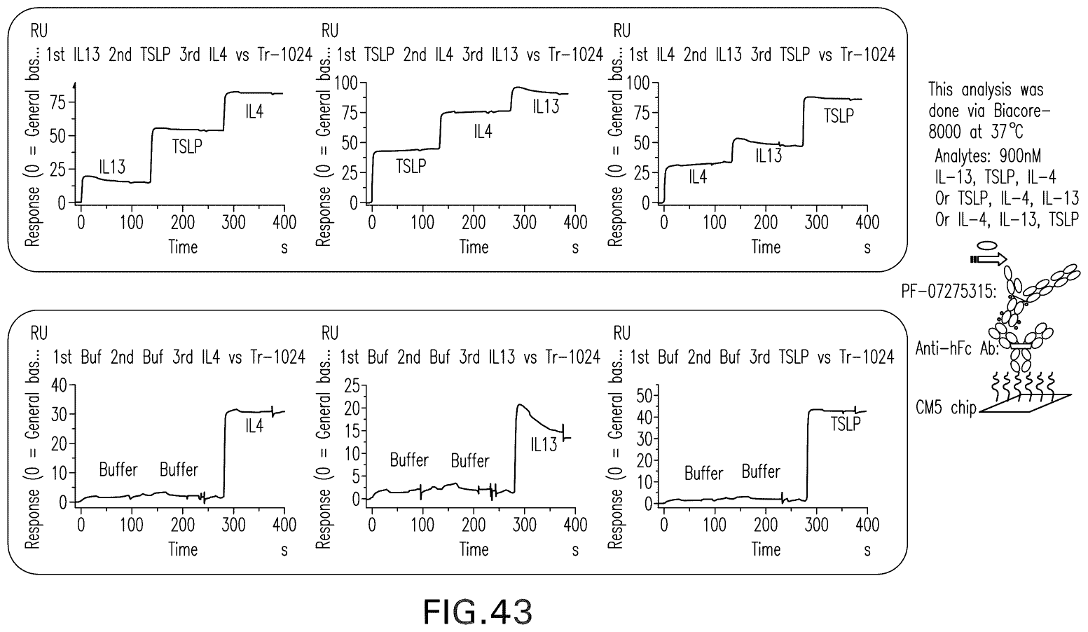

SPR was used to evaluate simultaneous binding of IL413TSLP-1024 to all three target cytokines, human IL-4, IL-13, and TSLP. The IL413TSLP-1024 trispecific was captured with an anti-human Fc antibody that was directly immobilized on a CM5 sensor chip. A high concentration of 900 nM human IL-4, IL-13, and TSLP were sequentially injected over the captured trispecific using three different injection orders and binding was detected by SPR as described in EXAMPLE 52. This analysis was done using a Biacore 8K instrument at a temperature of 37° C. Results confirmed that all three cytokines could simultaneously bind to the IL413TSLP-1024 trispecific regardless of the injection order (FIG. 43).

### Example 84

### Neutralization Activity of Trispecific IL413TSLP-1024 Compared to Tezepelumab

Anti-TSLP tezepelumab (Tezspire™; AMG157; MEDI9929; Amgen/MedImmune) has shown efficacy in asthma (46), and trends toward activity in AD (47). It is currently approved for treatment of severe asthma.

We compared the cytokine neutralization activity of trispecific IL413TSLP-1024 against tezepelumab (TSLP-0001) in the monocyte TARC production bioassay. The TSLP neutralization activity of IL413TSLP-1024 was comparable to that of tezepelumab (Table 78).

TABLE 78
TSLP Neutralization Activity of
IL413TSLP-1024 and Tezepelumab

| Cytokine | Antibody or Trispecific | | IC₅₀ (pM) ^{a} |
|---|---|---|---|
| rHu TSLP | Tezepelumab | Median | 19.15 |
| | | s.e.m. | 1.42 |
| | | n | 30 |
| | IL413TSLP-1024 | Median | 13.99 |
| | | s.e.m. | 1.49 |
| | | n | 11 |

^{a} Mononuclear cells isolated from human peripheral blood were incubated overnight at 37° C. with recombinant human TSLP (4 ng/ml; Pfizer), along with dilutions of the trispecific IL413TSLP-1024 or tezepelumab. TARC production in cell supernatants was quantitated by MSD.

### In Vivo Anti-Tumor Efficacy of Blocking IL-4, IL-13 and TSLP Alone or in Combination with Blocking PD1

### Example 85

### In Vivo Tumor Growth Inhibition of Blocking IL4, IL13 and TSLP Alone or in Combination with PD1 Blockade

Surrogate large molecules that block murine IL-4 (mAb clone 11B11), IL-13 (mIL13Rα2-mFc), TSLP (mAb clone 28F12) or PD1 (mAb clone F2: SEQ ID NO: 227 and SEQ ID NO: 228) were tested for their ability to inhibit tumor growth in vivo.

Subcutaneous CT26 tumor model format: a prior study demonstrated that a subcutaneous tumor implantation model using the CT26 colon carcinoma cell line responded to IL-4 blockade (97). This model was selected to compare the effects of blocking various combinations of IL-4, IL-13, TSLP and PD1 on tumor bearing mice, as described in the following experiment:

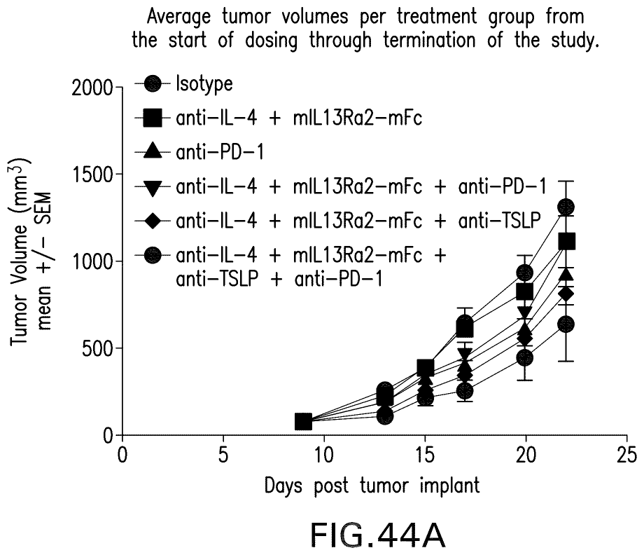

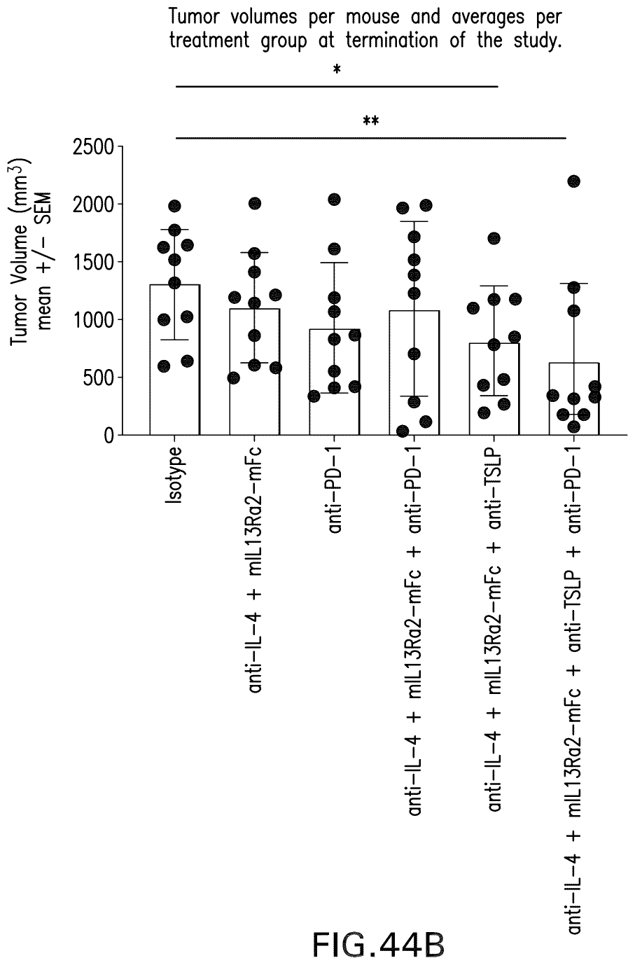

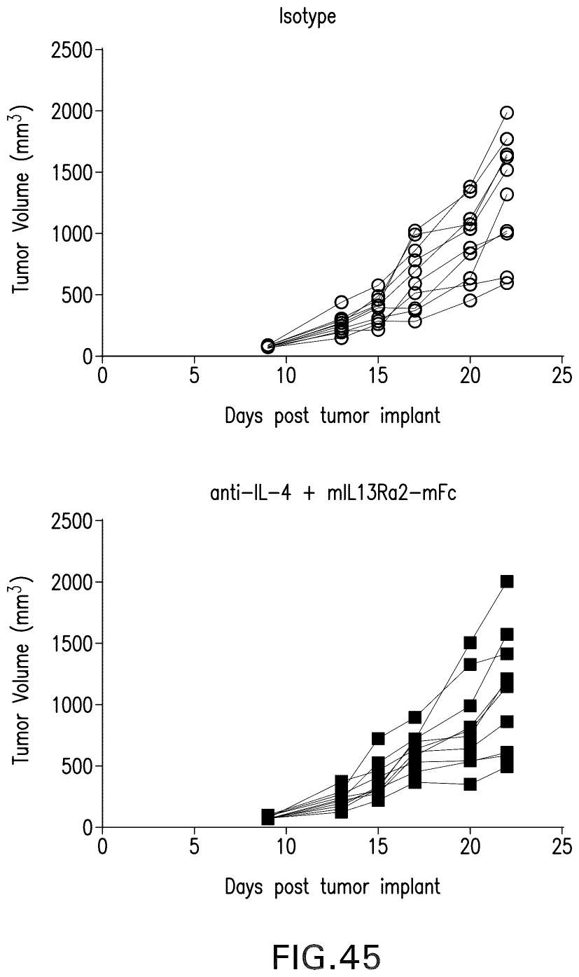

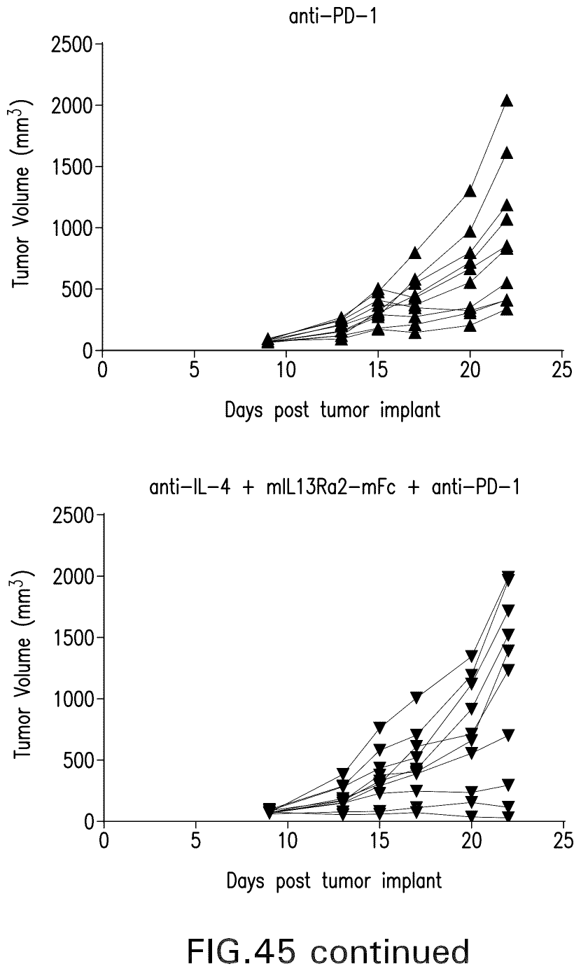

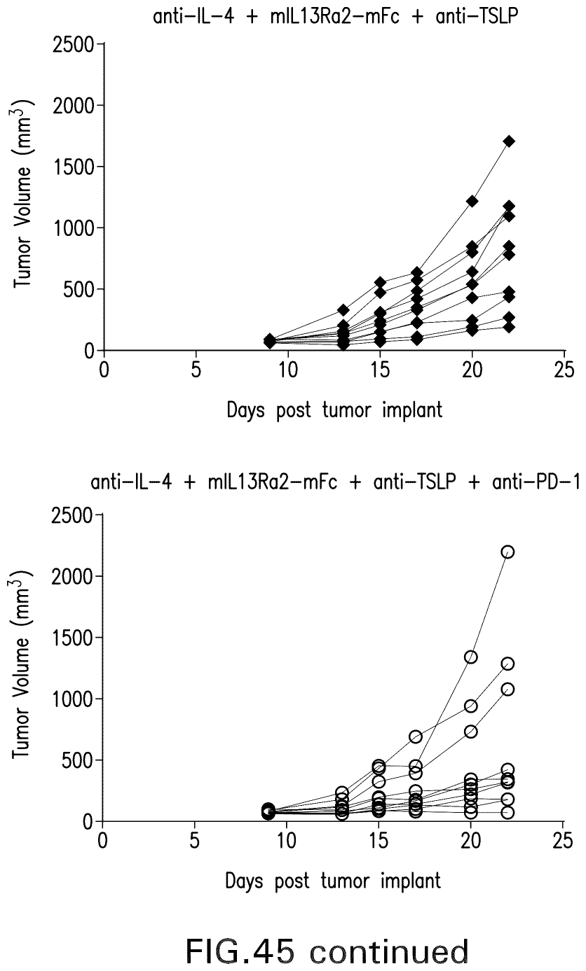

Female Balb/C J mice were subcutaneously implanted in the right hind flank with approximately 500,000 CT26 cells, which had been freshly thawed from a single, low passage vial (of 1,000,000 cells) and cultured for the minimum time necessary to establish sufficient cells for implantation. When sufficient animals with visible tumor masses of approximately 75 mm³ (as defined by (length*width²)/2) were obtained (defined as day 9), mice were randomized into groups (of n=10 animals) immediately prior to dosing. The treatment groups were: (1) isotype control, (2) anti-IL-4 plus mIL13Rα2-mFc, (3) anti-PD1, (4) anti-IL-4 plus mIL13Rα2-mFc plus anti-PD1, (5) anti-IL-4 plus mIL13Rα2-mFc plus anti-TSLP or (6) anti-IL-4 plus mIL13Rα2-mFc plus anti-TSLP plus anti-PD1 (summarized in Table 88). Anti-IL-4 (10 mg/kg) and mIL13Rα2-mFc (10 mg/kg) were injected subcutaneously (neck scruff) while anti-PD1 (10 mg/kg) and anti-TSLP (10 mg/kg) were injected intraperitoneally, every 3-4 days, for a total of 5 doses over 14 days, into each animal of a given group. Equal volumes of isotype-matched control antibodies were administered into each animal of a given group such that the total mass of proteins administered per dose were equivalent across treatment groups. Tumor volumes were tracked throughout the course of the experiment (FIG. 44A and FIG. 45). Animals were sacrificed on day 22 post-tumor implantation and tumor volumes of animals within each treatment group were recorded (FIG. 44). Tumor volumes were plotted in PRISM version 9.0.0 (Graphpad) and differences between treatment groups analyzed by ANCOVA with post-hoc, pair-wise Tukey's tests against the isotype control group. The results demonstrated that blocking the combination of IL-4, IL-13 and TSLP inhibited tumor growth alone and in combination with PD-1 antagonism.

TABLE 88
Study design of the subcutaneous CT26 study

| | | | Dose | Dosing | |
|---|---|---|---|---|---|
| Group | Drug | n/group | (mg/kg/dose) | Route | Regimen |
| 1 | Isotype (mIgG2a, mlgG1, | 10 | 8 | SC (mIgG2a) | Q3D × 5 |
| | rlGg2a) | | 10 | IP (rIgG2a, mIgG1) | |
| | | | 10 | | |
| 2 | Anti-mIL-4 (clone 11B11) + | 10 | 10 | SC | Q3D × 5 |
| | anti-IL-13 (mIL-13Ra2-mFc) | | 8 | SC | |
| 3 | Anti-PD-1 (clone F2) | 10 | 10 | IP | Q3D × 5 |
| 4 | Anti-IL-4 + | 10 | 10 | SC | Q3D × 5 |
| | anti-IL-13 + | | 8 | SC | |
| | anti-PD-1 | | 10 | IP | |
| 5 | Anti-IL-4 + | 10 | 10 | SC | Q3D × 5 |
| | anti-IL-13 + | | 8 | SC | |
| | anti-mTSLP (clone 28F12) | | 10 | SC | |
| 6 | Anti-IL-4 + | 10 | 10 | SC | Q3D × 5 |
| | anti-IL-13 + | | 8 | SC | |
| | anti-TSLP + | | 10 | SC | |
| | anti-PD-1 | | 10 | IP | |

### In Vitro Blocking IL-4 and TSLP Repolarize Primary Human T Cells Enabling Tumor Growth Control

### Example 86

### Blockade of IL-4 or TSLP Prevented Suppression of Interferon Gamma Secretion from Primary Human T Cells In Vitro

Type 1 polarization of 004 and 008 T cells is associated with immunological control of tumors and responses to immune checkpoint inhibitors (97-101). Interferon gamma production by T cells is a hallmark of type 1 polarization, and its production is associated with tumor growth inhibition (102, 103). Sequence optimized anti-IL4 clone 1040 and affinity optimized anti-TSLP clone 0875 were tested for their abilities to restore interferon gamma secretion from primary human CD4 and CD8 T cells activated in the presence of exogenous, recombinant human IL-4 or TSLP.

Bioassay format: polarization of primary human T cells that recognize A375 tumor cells. Exposure of human T cells to IL-4 or TSLP polarizes them towards type 2 responses and suppresses production of interferon gamma (99, 104-106). To test the abilities of mAb IL4-1040 and mAb TSLP-0875 to prevent suppression of interferon gamma secretion by tumor-reactive primary human T cells we used the following bioassay. Separate populations of CD4 and CD8 T cells were purified from cryopreserved peripheral blood mononuclear cells using magnetic beads (Stemcell Technologies). CD4 or CD8 T cells (500,000 cells/well) were separately co-cultured with mitomycin C inactivated A375 human tumor cells (500,000 cells/well). These cultures were conducted in 24-well G-Rex plates (Wilson Wolf) using X-Vivo 15 media (Lonza Whittaker) supplemented with 10% pooled human AB sera (Sigma-Aldrich) and recombinant human IL-2 (20 ng/mL) and IL-7 (10 ng/mL). For this experiment, some wells were treated with an irrelevant, isotype-matched antibody (1.5 pM), some were treated with this isotype control antibody (1.5 pM) and either recombinant human IL-4 (10 μg/mL) or long form TSLP (10 μg/mL), some were treated with recombinant human IL-4 (10 μg/mL) and mAb IL4-1040 (1.5 pM) and some were treated with recombinant human TSLP (10 μg/mL) and mAb TSLP-0875 (1.5 pM). Polarization and expansion of A375-reactive CD4 or CD8 T cells under these conditions occurred for 13 days at 37° C. in a 5% CO₂ incubator. At the end of this time, T cells were transferred to fresh media lacking cytokines and antibodies, counted, adjusted to 1,000,000 cells/mL and rested by incubation overnight at 37° C. in a 5% CO₂ incubator.

Bioassay format: restimulation of A375-reactive primary human T cells for measurement of interferon gamma secretion. A375 cells expressing nuclear-localized GFP were seeded at 5,000 cells/well in a black, optical bottom 96-well plate (Perkin Elmer) and incubated overnight at 37° C., 5% CO₂. Rested, A375-reactive CD4 and CD8 T cells described above were mixed and added to the plate containing GFP-expressing, live A375 tumor cells (50,000 CD4 and 50,000 CD8 T cells/well). These cultured occurred at 37° C. in a 5% CO₂ incubator for 5 days using X-Vivo 15 media supplemented with 10% pooled human AB sera. Some wells were treated with an irrelevant, isotype-matched antibody (1.5 pM), some were treated with this isotype control antibody (1.5 pM) and either recombinant human IL-4 (10 pg/mL) or long form TSLP (10pg/mL), some were treated with recombinant human IL-4 (10 pg/mL) and mAb IL4-1040 (1.5 pM) and some were treated with recombinant human TSLP (10 pg/mL) and mAb TSLP-0875 (1.5 pM). Treatment details are listed in Table 89. On day 5 the plate was centrifuged, and half of the media was collected to quantify interferon gamma protein by multiplex ELISA (Meso Scale Discovery). Concentrations of secreted cytokines were extrapolated from standard curves in Excel (Microsoft). Data were plotted and differences between groups tested by ANOVA with post-hoc Šidák's multiple comparison tests in PRISM version 9.0.0 (GraphPad).

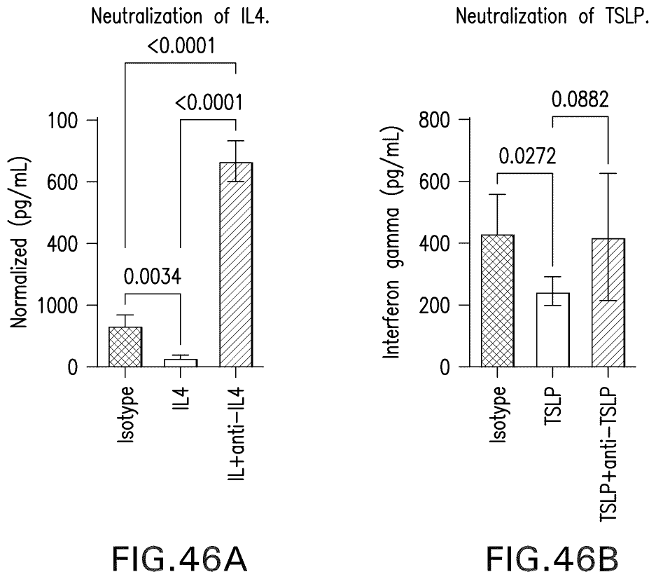

CD4 and CD8 T cells from six distinct donors were tested as described above. Addition of recombinant human IL-4 or TSLP to the assays described above reduced secretion of interferon gamma. Neutralization of IL-4 with mAb IL4-1040 prevented suppression of interferon gamma secretion and enhanced interferon gamma secretion above that of restimulated T cells that had been treated with only the irrelevant isotype control antibody (FIG. 46A). Neutralization of TSLP with mAb TSLP-0875 prevented suppression of interferon gamma secretion for some of the donors (FIG. 46B).

TABLE 89
Treatments used in the priming and restimulation
of primary human T cells described in Example 87.

| | | | Name in | | Restimula- |
|---|---|---|---|---|---|
| Treatment | Dose | | FIG. 46 | Priming | tion |
| Isotype antibody | 1.5 | pM | Isotype | Yes | Yes |
| Isotype antibody + | 1.5 | pM | IL4 | Yes | Yes |
| IL4 | 10 | pg/mL | | | |
| mAb IL4-1040 + | 1.5 | pM | IL4 + anti- | Yes | Yes |
| IL4 | 10 | pg/mL | IL4 | | |
| Isotype antibody + | 1.5 | pM | TSLP | Yes | Yes |
| TSLP | 10 | pg/mL | | | |
| mAb TSLP-0875 + | 1.5 | pM | TSLP + anti- | Yes | Yes |
| TSLP | 10 | pg/mL | TSLP | | |

### Example 87

### Blockade of IL4 or TSLP Improved T Cell-Mediated Control of Growth by a Human Cancer Cell Line In Vitro

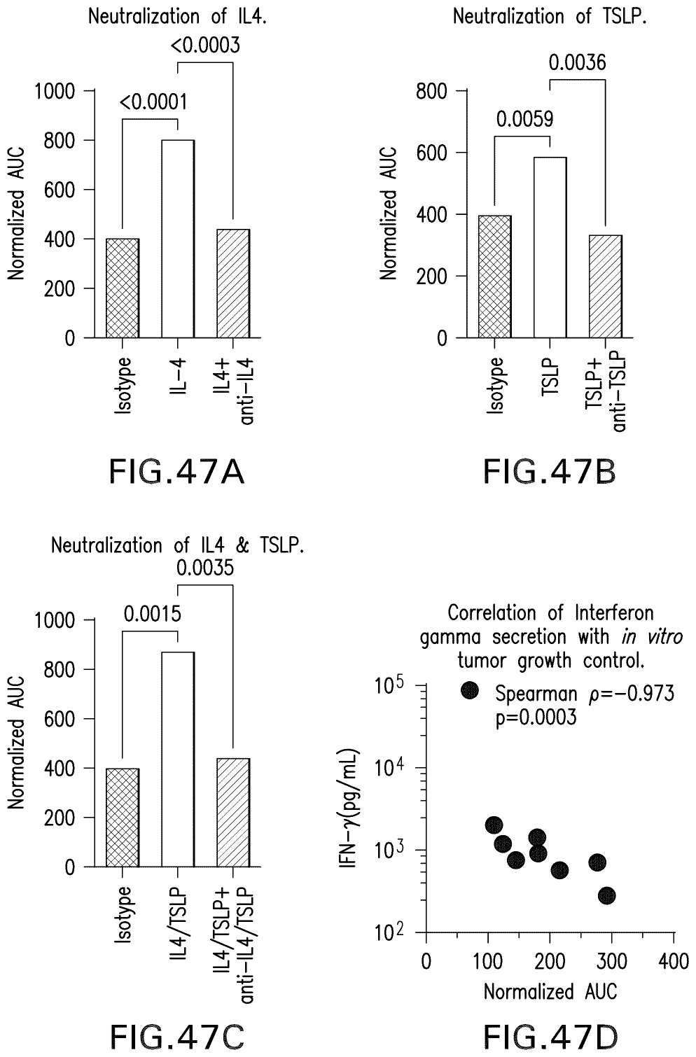

Primary human CD4 and CD8 T cells were polarized and restimulated as described in Example 87. Counts of GFP-expressing A375 cells were made every three hours using an Incucyte S3 (Sartorius). Cell counts at each time point were normalized to the initial cell count. These normalized longitudinal counts were plotted and the areas under the curves (AUC) were calculated for each treatment group per donor. MAb IL4-1040 reversed suppression of tumor growth inhibition by exogenous recombinant IL4 (FIG. 47A), mAb TSLP-0875 reversed suppression of tumor growth inhibition by exogenous recombinant TSLP (FIG. 47B) and the combination of mAbs IL4-1040 and TSLP-0875 reversed tumor growth inhibition by exogenous recombinant IL4 and TSLP (FIG. 47C). There was a significant, inverse correlation between the concentration of Interferon gamma secreted into the media and the number of tumor cells (FIG. 47D). Data were plotted, differences between groups by ANOVA with post-hoc Šidák's multiple comparison tests and Spearman correlation were performed in PRISM version 9.0.0 (GraphPad). Taking together, the data show that neutralization of IL-4 by mAb IL4-1040 and/or TSLP by mAb TSLP-0875 enhanced T cell control of A375 human cancer cell line growth in vitro.

### Example 88

### Recombinant IL-4 and TSLP Reduce the Proportion of In Vitro Activated Primary Human CD8 T Cells Expressing the Cytotoxic Proteins Perforin and Granzyme B

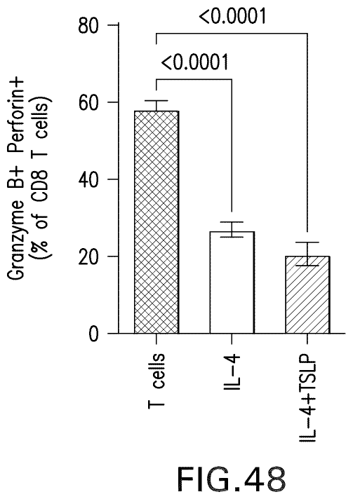

Primary human CD8 T cells were polarized as described in Example 87 either without exogenous cytokine or in the presence of 0.8 ng/mL recombinant human IL-4 or 0.8 ng/mL each recombinant human IL-4 and TSLP. After polarization the CD8 T cells were counted and 250,000 from each condition were collected for analysis by flow cytometry. The cells were washed with cold PBS and labeled with Live/Dead Aqua Viability Dye (ThermoFisher) in the dark at room temperature for 20 minutes. After washing in Flow Cytometry Staining Buffer (ThermoFisher) cells were stained with R718-labeled anti-human CD3, BUV496-labeled anti-human CD4 and BUV737-labeled anti-human CD8 (all from BD Biosciences) at 4° C. for 20 minutes. The cells were again washed with Flow Cytometry Staining Buffer and fixed with FoxP3/Transcription Factor Fixation/Permeabilization solution (ThermoFisher) for 20 minutes at room temperature. Cells were then washed with Permeabilization Buffer (ThermoFisher) and stained with APC-labeled anti-human Granzyme B and BV421-labeled anti-human Perforin (both from Biolegend) for 20 minutes in the dark at room temperature. Single, live CD8 T cells were gated based on forward and side light scatter, low Live/Dead Aqua signal, and expression of CD3 and CD8. Expression of both Perforin and Granzyme B by CD8 T cells was determined by flow cytometry, and the percentage of double-positive cells quantified with FlowJo software (BD Biosciences). Both recombinant human IL-4 alone and the combination of recombinant human IL-4 and TSLP decreased the proportion of A375 polarized primary human CD8 T cells expressing both cytotoxic proteins Perforin and Granzyme B (FIG. 48). Data were plotted and differences between groups tested by ANOVA with post-hoc Šidák's multiple comparison tests in PRISM version 9.0.0 (GraphPad).

In conclusion: recombinant human IL-4 and/or TSLP decreased the proportion of tumor-reactive primary human CD8 T cells expressing the cytotoxic proteins Perforin and Granzyme B. These cytokines alone or in combination also reduced Interferon gamma secretion by polarized and restimulated primary human T cells, and impaired control of A375 tumor cell growth by these T cells in vitro. Neutralizing exogenous recombinant IL-4 with mAb IL4-1040 and/or exogenous recombinant TSLP with mAb TSLP-0875 during polarization and reactivation either reversed or trended towards reversing the reduction in Interferon gamma secretion and tumor growth control caused by these cytokines.

### In Vitro Blocking IL-4 and IL13 Reduce Secretion of the Chemokine CCL17/TARC by Human Clear Cell Renal Carcinoma Cells

### Example 89

### IL-4 Neutralization Activity of Trispecifics IL413TSLP-1028 and IL413TSLP-1037 in a Cell-Based Assay

Serum levels of the chemokine CCL17/Thymus and Activation Regulated Chemokine (TARC) are used as a pharmacodynamic biomarker in clinical trials of antibodies that block IL-4 and IL-13 signaling (107). Expression of CCL17 is also associated with immunosuppressive tumor microenvironments (108-110). The IL-4 and IL-13 neutralization activities of trispecific IL413TSLP-1028 and IL413TSLP-1037 were tested in the following bioassay.

769-P IL-4 and IL-13 CCL17 Bioassay Format: for this assay, 769-P human clear cell renal cell carcinoma cells (American Type Culture Collection, Manassas, VA) were grown as an adherent monolayer in 96-well plates seeded at 5×10\hat{ }5 cells per well. To test antibody inhibition of cytokine responses, the pre-determined EC₉₀ concentration of 30 ng/mL recombinant human IL-13 or 1 ng/mL recombinant human IL-4 was added. Trispecific antibodies were used at varying concentrations ranging from 6.7 to 0.0523 nM in wells that received IL-4 or 23 to 0.1797 nM in wells that received IL-13. Cells were incubated at 37° C. for approximately 20 hours after which conditioned media was collected and levels of secreted proteins assayed by Legendplex fluid-phase multiplex immunoassays (Biolegend). Concentrations of secreted cytokines were extrapolated from standard curves using the cloud-based tool provided by Biolegend. IC₅₀ values were calculated from antibody dose titration data using PRISM version 9.0.0 (GraphPad).

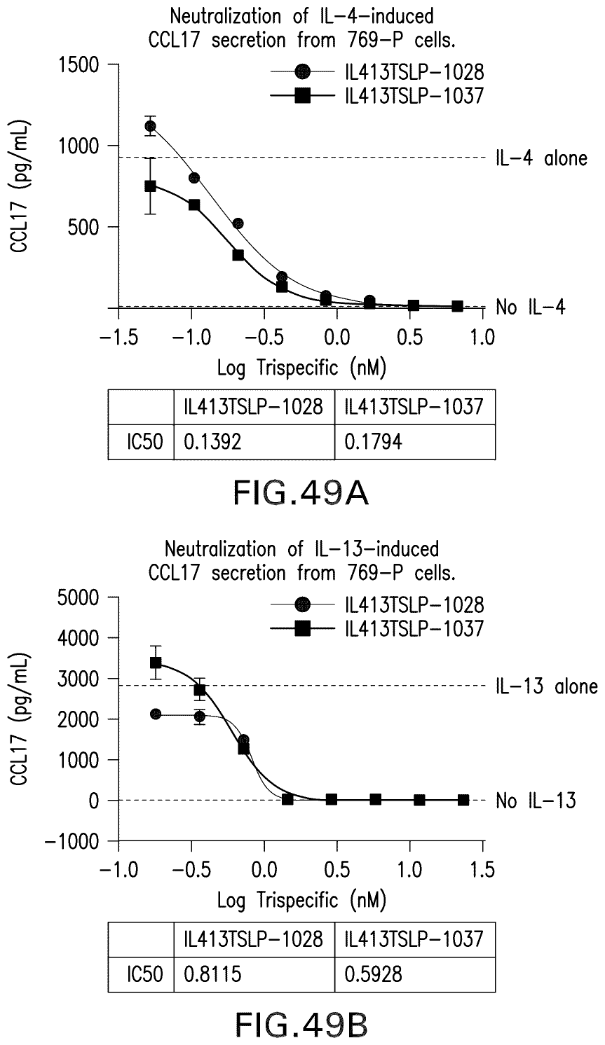

IL-4 Neutralization Activity of IL413TSLP-1028 and IL413TSLP-1037: trispecific IL413TSLP-1037 is comprised of IL-13 binding domain 0001 (1RVHC9 VLA4), IL-4 binding domain 1040, and TSLP binding domain 0855. Trispecific IL413TSLP-1028 is comprised of IL-13 binding domain 0001 (1RVHC9 VLA4), IL-4 binding domain 1040, and TSLP binding domain 0871. IL413TSLP-1037 neutralized IL-4-induced CCL17 secretion by 769-P cells in a dose-dependent fashion (FIG. 49A). The IC₅₀ of IL413TSLP-1037 in this assay was 0.1794 nM. IL413TSLP-1028 neutralized IL-4-induced CCL17 secretion by 769-P cells in a dose-dependent fashion (FIG. 49A). The IC₅₀ of IL413TSLP-1028 in this assay was 0.1392 nM.

IL-13 Neutralization Activity of IL413TSLP-1037 and IL413TSLP-1028: IL413TSLP-1037 neutralized IL-13-induced CCL17 secretion by 769-P cells in a dose-dependent fashion (FIG. 49B). The IC₅₀ of IL413TSLP-1037 in this assay was 0.5928 nM. IL413TSLP-1028 neutralized IL-4-induced CCL17 secretion by 769-P cells in a dose-dependent fashion (FIG. 49B). The IC₅₀ of IL413TSLP-1028 in this assay was 0.8115 nM.

In conclusion: trispecifics IL413TSLP-1037 and IL413TSLP-1028 are potent neutralizers of human IL-4 and IL-13 bioactivity in this human tumor cell line-based assay.

### REFERENCES

1. US 2015/0322135 A1. Engineered monomeric antibody fragments. Ishino Tetsuya et al.
2. Mechold U, et al. Codon optimization of the BirA enzyme gene leads to higher expression and an improved efficiency of biotinylation of target proteins in mammalian cells. J Biotechnol. 2005; 116(3):245-9.
3. US 2009/0238823 AI. antigen binding proteins capable of binding thymic stromal lymphopoietin. Micheael R. Comeau et al.
4. Verstraete K, et al, Structure and antagonism of the receptor complex mediated by human TSLP in allergy and asthma Nat Commun. 2017 Apr. 3; 8:14937. doi: 10.1038/ncomms14937.
5. Brian J Fennell, et al. CDR-restricted engineering of native human scFvs creates highly stable and soluble bifunctional antibodies for subcutaneous delivery. MAbs 2013, 5, 882-895.
6. Adam Root et al, Development of PF-06671008, a Highly Potent Anti-P-cadherin/Anti-CD3 Bispecific DART Molecule with Extended Half-Life for the Treatment of Cancer. Antibodies 2016, 5(1), 6
7. Lindsay B. Avery et al. Establishing in vitro in vivo correlations to screen monoclonal antibodies for physicochemical properties related to favorable human pharmacokinetics mAbs, 2018 10:2, 244-255,
8. Avery L B, et al. Utility of a human FcRn transgenic mouse model in drug discovery for early assessment and prediction of human pharmacokinetics of monoclonal antibodies. mAbs. 2016; 8:1064-1078.
9. U.S. Pat. No. 5,731,168. Paul J. Carter, Leonard G. Presta, John B. Ridgway. Method for making heteromultimeric polypeptides.
10. U.S. Pat. No. 7,183,076. Method for making multispecific antibodies having heteromultimeric and common components.
11. WO 2015/173756A2. Bispecific antibodies.
12. Pavel Strop, et al. Generating Bispecific Human IgG1 and IgG2 Antibodies from Any Antibody Pair. J. Mol. Biol. (2012) 420, 204-219
13. Sarah Sirin, et al. AB-Bind: Antibody binding mutational database for computational affinity predictions. Protein Sci. 2016 February; 25(2): 393-409.
14. Guerois R, et al. (2002) Predicting changes in the stability of proteins and protein complexes: a study of more than 1000 mutations. J Mol Biol 320:369-387.
15. Schymkowitz J, et al (2005) The FoldX web server: an online force field. Nucleic Acids Res 33: W382-W38
16. Sharma V K, et al. In silico selection of therapeutic antibodies for development: viscosity, clearance, and chemical stability. Proceedings of the National Academy of Sciences of the United States of America. 2014; 111(52):18601-6.
17. Tomar D S, et al. In-silico prediction of concentration-dependent viscosity curves for monoclonal antibody solutions. mAbs. 2017; 9(3):476-89.
18. Apgar J R, et al. (2020) Modeling and mitigation of high-concentration antibody viscosity through structure-based computer-aided protein design. PLoS ONE 15(5): e0232713. https://doi. org/10.1371/journal.pone.0232713T
19. Chow C K, et al. Therapeutic Antibody Engineering To Improve Viscosity and Phase Separation Guided by Crystal Structure. Molecular pharmaceutics. 2016; 13(3):915-23.
20. Geoghegan J C, et al. Mitigation of reversible self-association and viscosity in a human IgG1 monoclonal antibody by rational, structure-guided Fv engineering. mAbs. 2016; 8(5):941-50.
21. Nichols P, et al. Rational design of viscosity reducing mutants of a monoclonal antibody: hydrophobic versus electrostatic inter-molecular interactions. mAbs. 2015; 7(1):212-30.
22. Yadav S, et al. The influence of charge distribution on self-association and viscosity behavior of monoclonal antibody solutions. Molecular Pharmaceutics. 2012; 9(4):791-802.
23. Agrawal N J, et al. Computational tool for the early screening of monoclonal antibodies for their viscosities. mAbs. 2016; 8(1):43-8.
24. U.S. Pat. No. 6,902,734.
25. Cohen E S, et al. Oxidation of the alarmin IL-33 regulates ST2-dependent inflammation. Nat Commun 2015; 6:8327-36.
26. Schafer J R, et al. Prediction of well-conserved HIV-1 ligands using a matrix-based algorithm, EpiMatrix. Vaccine 1998; 16 (19): 1880-1884.
27. Wang P, et al. Peptide binding predictions for HLA DR, DP and DQ molecules. BMC Bioinformatics. 2010; 11.1:568.
28. Junttila, I. S. 2018. Tuning the Cytokine Responses: An Update on Interleukin (IL)-4 and IL-13 Receptor Complexes. Front Immunol. 9: 888.
29. Wills-Karp, M. et al. 2008. Untangling the complex web of IL-4- and IL-13-mediated signaling pathways. Sci Signal. 1: pe55.
30. de Waal Malefyt, R., et al. 1993. Effects of IL-13 on phenotype, cytokine production, and cytotoxic function of human monocytes. Comparison with IL-4 and modulation by IFN-gamma or IL-10. J Immunol. 151: 6370-81.
31. Vercelli, D., et al. 1988. Human recombinant interleukin 4 induces Fc epsilon R2/CD23 on normal human monocytes. J Exp Med. 167: 1406-16.
32. Wu, J. et al. 2020. Efficacy of biologics in atopic dermatitis. Expert Opin Biol Ther.
33. Shirley, M. 2017. Dupilumab: First Global Approval. Drugs. 77: 1115-1121.
34. Harb, H. et al. 2019. Mechanisms of Dupilumab. Clin Exp Allergy.
35. DeMeo, D. L., C. Lange, E. K. Silverman, J. M. Senter, J. M. Drazen, M. J. Barth, N. Laird, and S. T. Weiss. 2002. Univariate and multivariate family-based association analysis of the IL-13 ARG130GLN polymorphism in the Childhood Asthma Management Program. Genet Epidemiol. 23: 335-48.
36. Heinzmann, A., et al. 2000. Genetic variants of IL-13 signalling and human asthma and atopy. Hum Mol Genet. 9: 549-59.
37. Kakkar, R. et al. 2008. The IL-33/ST2 pathway: therapeutic target and novel biomarker. Nat Rev Drug Discov. 7: 827-40.
38. Smithgall, M. D., et al. 2008. IL-33 amplifies both Th1- and Th2-type responses through its activity on human basophils, allergen-reactive Th2 cells, iNKT and NK cells. Int Immunol. 20: 1019-30.
39. Yang, Q., et al. 2011. IL-33 synergizes with TCR and IL-12 signaling to promote the effector function of CD8+ T cells. Eur J Immunol. 41: 3351-60.
40. Divekar, R. et al. 2015. Recent advances in epithelium-derived cytokines (IL-33, IL-25, and thymic stromal lymphopoietin) and allergic inflammation. Curr Opin Allergy Clin Immunol. 15: 98-103.
41. Hammad, H. et al. 2015. Barrier Epithelial Cells and the Control of Type 2 Immunity. Immunity. 43: 29-40.
42. Ziegler, S. F. et al. 2010. Sensing the outside world: TSLP regulates barrier immunity. Nat Immunol. 11: 289-93.
43. Liu, Y. J., et al. 2007. TSLP: an epithelial cell cytokine that regulates T cell differentiation by conditioning dendritic cell maturation. Annu Rev Immunol. 25: 193-219.
44. Varricchi, G., et al. 2018. Thymic Stromal Lymphopoietin Isoforms, Inflammatory Disorders, and Cancer. Front Immunol. 9: 1595.
45. Bjerkan, L., et al. 2016. Multiple Functions of the New Cytokine-Based Antimicrobial Peptide Thymic Stromal Lymphopoietin (TSLP). Pharmaceuticals (Basel). 9.
46. Corren, J., et al. 2017. Tezepelumab in Adults with Uncontrolled Asthma. N Engl J Med. 377: 936-946.
47. Simpson, E. L., et al. 2019. Tezepelumab, an anti-thymic stromal lymphopoietin monoclonal antibody, in the treatment of moderate to severe atopic dermatitis: A randomized phase 2a clinical trial. J Am Acad Dermatol. 80: 1013-1021.
48. May, R. D. et al. 2015. Strategies targeting the IL-4/IL-13 axes in disease. Cytokine. 75: 89-116.
49. Roesner, L. M., et al. 2019. Common and different roles of IL-4 and IL-13 in skin allergy and clinical implications. Curr Opin Allergy Clin Immunol. 19: 319-327.
50. Perkins, C., et al. 2006. IL-4 induces IL-13-independent allergic airway inflammation. J Allergy Clin Immunol. 118: 410-9.
51. Munitz, A., et al. 2008. Distinct roles for IL-13 and IL-4 via IL-13 receptor alpha1 and the type II IL-4 receptor in asthma pathogenesis. Proc Natl Acad Sci USA. 105: 7240-5.
52. Lupardus, P. J., et al. 2010. Molecular basis for shared cytokine recognition revealed in the structure of an unusually high affinity complex between IL-13 and IL-13Ralpha2. Structure. 18: 332-42.
53. Kawakami, K., et al. 2001. The interleukin-13 receptor alpha2 chain: an essential component for binding and internalization but not for interleukin-13-induced signal transduction through the STAT6 pathway. Blood. 97: 2673-9.
54. Bachert, C., et al. 2019. Efficacy and safety of dupilumab in patients with severe chronic rhinosinusitis with nasal polyps (LIBERTY NP SINUS-24 and LIBERTY NP SINUS-52): results from two multicentre, randomised, double-blind, placebo-controlled, parallel-group phase 3 trials. Lancet.
55. Blauvelt, A et al. 2017. Long-term management of moderate-to-severe atopic dermatitis with dupilumab and concomitant topical corticosteroids (LIBERTY AD CHRONOS): a 1-year, randomised, double-blinded, placebo-controlled, phase 3 trial. Lancet. 389: 2287-2303.
56. Busse, W. W., et al. 2018. Liberty Asthma QUEST: Phase 3 Randomized, Double-Blind, Placebo-Controlled, Parallel-Group Study to Evaluate Dupilumab Efficacy/Safety in Patients with Uncontrolled, Moderate-to-Severe Asthma. Adv Ther. 35: 737-748.
57. Guttman-Yassky et al. 2020. Efficacy and Safety of Lebrikizumab, a High-Affinity Interleukin 13 Inhibitor, in Adults With Moderate to Severe Atopic Dermatitis: A Phase 2b Randomized Clinical Trial. JAMA Dermatol. 156: 411-420.
58. Wollenberg, A., et al. 2021. Tralokinumab in atopic dermatitis. J Dtsch Dermatol Ges. 19: 1435-1442.
59. Silverberg, J. I., et al. 2021. Tralokinumab plus topical corticosteroids for the treatment of moderate-to-severe atopic dermatitis: results from the double-blind, randomized, multicentre, placebo-controlled phase Ill ECZTRA 3 trial. Br J Dermatol. 184: 450-463.
60. Hanania, N. A., P. et al. 2016. Efficacy and safety of lebrikizumab in patients with uncontrolled asthma (LAVOLTA I and LAVOLTA II): replicate, phase 3, randomised, double-blind, placebo-controlled trials. Lancet Respir Med. 4: 781-796.
61. Panettieri, R. A., Jr., et al. 2018. Tralokinumab for severe, uncontrolled asthma (STRATOS 1 and STRATOS 2): two randomised, double-blind, placebo-controlled, phase 3 clinical trials. Lancet Respir Med. 6: 511-525.
62. Seger, E. W., et al. 2019. Relative efficacy of systemic treatments for atopic dermatitis. J Am Acad Dermatol. 80: 411-416 e4.
63. Silverberg, J. I., et al. 2021. Comparative efficacy and safety of systemic therapies used in moderate-to-severe atopic dermatitis: a systematic literature review and network meta-analysis. J Eur Acad Dermatol Venereol. 35: 1797-1810.
64. Adhikary, P. P., et al. 2021. TSLP as druggable target—a silver-lining for atopic diseases? Pharmacol Ther. 217: 107648.
65. Menzies-Gow, et al. 2021. Tezepelumab in Adults and Adolescents with Severe, Uncontrolled Asthma. N Engl J Med. 384: 1800-1809.
66. Cayrol, C. et al. 2014. IL-33: an alarmin cytokine with crucial roles in innate immunity, inflammation and allergy. Curr Opin Immunol. 31: 31-7.
67. De la Fuente, M., T et al. 2015. The IL-33/ST2 axis: Role in health and disease. Cytokine Growth Factor Rev. 26: 615-23.
68. Homsak, E. et al. 2020. Soluble ST2: A complex and diverse role in several diseases. Clin Chim Acta. 507: 75-87.
69. Imai, Y. 2019. Interleukin-33 in atopic dermatitis. J Dermatol Sci.
70. Yang, N., et al. 2021. Novel Targeted Biological Agents for the Treatment of Atopic Dermatitis. BioDrugs. 35: 401-415.
71. Nnane, I., et al. 2020. The first-in-human study of CNTO 7160, an anti-interleukin-33 receptor monoclonal antibody, in healthy subjects and patients with asthma or atopic dermatitis. Br J Clin Pharmacol. 86: 2507-2518.
72. Wechsler, M. E., et al. 2021. Efficacy and Safety of Itepekimab in Patients with Moderate-to-Severe Asthma. N Engl J Med. 385: 1656-1668.
73. Rabe, K. F., et al. 2021. Safety and efficacy of itepekimab in patients with moderate-to-severe COPD: a genetic association study and randomised, double-blind, phase 2a trial. Lancet Respir Med. 9: 1288-1298.
74. Nguyen, J. K., et al. 2020. The IL-4/IL-13 axis in skin fibrosis and scarring: mechanistic concepts and therapeutic targets. Arch Dermatol Res. 312: 81-92.
75. Teng M W, et al. IL-12 and IL-23 cytokines: from discovery to targeted therapies for immune-mediated inflammatory diseases. Nat Med. 2015 July; 21(7):719-29.
76. Langrish C L, et al. IL-12 and IL-23: master regulators of innate and adaptive immunity. Immunol Rev. 2004 December; 202:96-105.
77. Tuzlak S, et al. Repositioning T_{H} cell polarization from single cytokines to complex help. Nat Immunol. 2021 October; 22(10):1210-1217.
78. Noda S, et al. The translational revolution and use of biologics in patients with inflammatory skin diseases. J Allergy Clin Immunol. 2015 February; 135(2):324-36.
79. Khattri S, et al. Efficacy and safety of ustekinumab treatment in adults with moderate-to-severe atopic dermatitis. Exp Dermatol. 2017 January; 26(1):28-35.
80. Saeki H, et al. Efficacy and safety of ustekinumab in Japanese patients with severe atopic dermatitis: a randomized, double-blind, placebo-controlled, phase II study. Br J Dermatol. 2017 August; 177(2):419-427.
81. Czarnowicki T, et al. Atopic dermatitis endotypes and implications for targeted therapeutics. J Allergy Clin Immunol. 2019 January; 143(1):1-11.
82. Svenningsen S, et al. Asthma Endotypes and an Overview of Targeted Therapy for Asthma. Front Med (Lausanne). 2017 Sep. 26; 4:158.
83. Allanore Y, et al. A randomised, double-blind, placebo-controlled, 24-week, phase II, proof-of-concept study of romilkimab (SAR156597) in early diffuse cutaneous systemic sclerosis. Ann Rheum Dis. 2020 December; 79(12):1600-1607.
84. Radstake T R, et al. The pronounced Th17 profile in systemic sclerosis (SSc) together with intracellular expression of TGFbeta and IFNgamma distinguishes SSc phenotypes. PLoS One. 2009 Jun. 17; 4(6):e5903.
85. Yang X, et al. Increased frequency of Th17 cells in systemic sclerosis is related to disease activity and collagen overproduction. Arthritis Res Ther. 2014 Jan. 7; 16(1):R4.
86. López-Isac E, et al. Identification of IL12RB1 as a novel systemic sclerosis susceptibility locus. Arthritis Rheumatol. 2014 December; 66(12):3521-3.
87. Lake A D, et al. Analysis of global and absorption, distribution, metabolism, and elimination gene expression in the progressive stages of human nonalcoholic fatty liver disease. Drug Metab Dispos. 2011 October; 39(10):1954-60.
88. Suárez-Farinas M, et al. Alopecia areata profiling shows TH1, TH2, and IL-23 cytokine activation without parallel TH17/TH22 skewing. J Allergy Clin Immunol. 2015 November; 136(5):1277-87.
89. Guttman-Yassky E, et al. Extensive alopecia areata is reversed by IL-12/IL-23p40 cytokine antagonism. J Allergy Clin Immunol. 2016 January; 137(1):301-304.
90. Guttman-Yassky E, et al. Phase 2a randomized clinical trial of dupilumab (anti-IL-4Ra) for alopecia areata patients. Allergy. 2021 Aug. 30.
91. Choy D F, et al. TH2 and TH17 inflammatory pathways are reciprocally regulated in asthma. Sci Transl Med. 2015 Aug. 19; 7(301):301ra129.
92. Eming S A, et al. Inflammation and metabolism in tissue repair and regeneration. Science. 2017 Jun. 9; 356(6342):1026-1030.
93. Parker J J, et al. Psoriasiform dermatitis during dupilumab treatment for moderate-to-severe atopic dermatitis in children. Pediatr Dermatol. 2021 November; 38(6):1500-1505.
94. Bridgewood C, et al. Unexpected connections of the IL-23/IL-17 and IL-4/IL-13 cytokine axes in inflammatory arthritis and enthesitis. Semin Immunol. 2021 Nov. 16: 101520.
95. Bakker D S, et al. Conjunctival inflammation in dupilumab-treated atopic dermatitis comprises a multicellular infiltrate with elevated T1/T17 cytokines: A case series study. Allergy. 2021 December; 76(12):3814-3817.
96. Chan B C L, et al. IL-33: Roles in Allergic Inflammation and Therapeutic Perspectives. Front. Immunol., 4 Mar. 2019.
97. Ito S E, et al. IL-4 blockade alters the tumor microenvironment and augments the response to cancer immunotherapy in a mouse model. Cancer Immunol Immunother 66, 1485-1496 (2017).
98. Villacres M C, Bergmann C C. Enhanced cytotoxic T cell activity in IL-4-deficient mice. J Immunol 162, 2663-2670 (1999).
99. Riou C, et al. IL-4 influences the differentiation and the susceptibility to activation-induced cell death of human naive CD8+ T cells. Int Immunol 18, 827-835 (2006).
100. Kemp R A, Ronchese F. Tumor-specific Tc1, but not Tc2, cells deliver protective antitumor immunity. J Immunol 167, 6497-6502 (2001).
101. Maier B, et al. A conserved dendritic-cell regulatory program limits antitumour immunity. Nature 580, 257-262 (2020).
102. Cui C, et al. Ratio of the interferon-gamma signature to the immunosuppression signature predicts anti-PD-1 therapy response in melanoma. Npj Genomic Medicine 6, (2021).
103. Karachaliou N, et al. Interferon gamma, an important marker of response to immune checkpoint blockade in non-small cell lung cancer and melanoma patients. Ther Adv Med Oncol 10, 1758834017749748 (2018).
104. De Monte L, et al. Intratumor T helper type 2 cell infiltrate correlates with cancer-associated fibroblast thymic stromal lymphopoietin production and reduced survival in pancreatic cancer. J Exp Med 208, 469-478 (2011).
105. Tatsuno K, et al. TSLP Directly Interacts with Skin-Homing Th2 Cells Highly Expressing its Receptor to Enhance IL-4 Production in Atopic Dermatitis. J Invest Dermatol 135, 3017-3024 (2015).
106. Rengarajan J, et al. Transcriptional regulation of Th1/Th2 polarization. Immunol Today 21, 479-483 (2000).
107. Hamilton J D, et al. Dupilumab suppresses type 2 inflammatory biomarkers across multiple atopic, allergic diseases. Clin Exp Allergy 51, 915-931 (2021).
108. Maruyama T, et al. CCL17 and CCL22 chemokines within tumor microenvironment are related to infiltration of regulatory T cells in esophageal squamous cell carcinoma. Dis Esophagus 23, 422-429 (2010).
109. Mizukami Y, et al. CCL17 and CCL22 chemokines within tumor microenvironment are related to accumulation of Foxp3+ regulatory T cells in gastric cancer. Int J Cancer 122, 2286-2293 (2008).
110. Kakinuma T, et al. Thymus and activation-regulated chemokine (TARC/CCL17) in mycosis fungoides: serum TARC levels reflect the disease activity of mycosis fungoides. J Am Acad Dermatol 48, 23-30 (2003).

TABLE 80
IL-4 antibodies All antibodies CH1: SEQ ID NO: 6, Hinge Region: SEQ ID NO: 7: CH2: SEQ ID NO: 8; CH3: SEQ ID NO: 9; JK Region (LC): SEQ ID
NO: 14; CL: SEQ ID NO: 16. SEQ ID numbers in parenthesis after specific sequence and without parenthesis where repeated. Mutations in reference
to IL4-1284

| IL-4 | | | | | | | | | | | |
|---|---|---|---|---|---|---|---|---|---|---|---|
| Antibody | CDR H1 | CDR H2 | CDRH3 | JH | V_{H} | HC | CDR L1 | CDR L2 | CDR L3 | V_{L} | LG |
| IL4-1284 | GFSLSTSGMGVS (1) | HIYWDQDKRYNPSLKS | 3 | 4 | 5 | 10 | KASQSVDYDGDSYMN | 12 | QQSNEDPPR (13) | 15 | 17 |
| | | (2) | | | | | (11) | | | | |
| IL4-1285 | 1 | 2 | 3 | 4 | 5 | 10 | 11 | 12 | R95T | | |
| IL4-1286 | 1 | 2 | 3 | 4 | 5 | 10 | 11 | 12 | 13 | | |
| IL4-1305 | 18 | 2 | 3 | 4 | 19 | | Y27dE | 12 | R95T | 20 | 197 |
| IL4-0002 | 18 | 2 | 3 | 21 | 22 | 23 | Y27dE | 12 | R95T | 20 | 197 |
| IL4-1040 | GFSLSNFGEGLS (18) | 2 | 3 | 21 | 22 | 23 | RASQSVDEEGDSYMN | 12 | QQSNKDPPT (25) | 26 | 27 |
| | | | | | | | (24) | | | | |
| IL4-1287 | S32F M34T | 2 | 3 | 4 | | | 11 | 12 | R95T | | |
| IL4-1288 | S32W M34S | 2 | 3 | 4 | | | 11 | 12 | R95T | | |
| IL4-1289 | T31N S32W | 2 | 3 | 4 | | | 11 | 12 | R95T | | |
| IL4-1291 | S30L S32F | 2 | 3 | 4 | | | 11 | 12 | R95T | | |
| IL4-1292 | S30L T31A S32W M34E | 2 | 3 | 4 | | | 11 | 12 | R95T | | |
| | V35al | | | | | | | | | | |
| IL4-1293 | S30L S32F M34S | 2 | 3 | 4 | | | 11 | 12 | R95T | | |
| IL4-1294 | S32W M34E | 2 | 3 | 4 | | | 11 | 12 | R95T | | |
| IL4-1295 | T31D S32F | 2 | 3 | 4 | | | 11 | 12 | R95T | | |
| IL4-1297 | S32F M34V | 2 | 3 | 4 | | | 11 | 12 | R95T | | |
| IL4-1298 | 18 | 2 | 3 | 4 | | | 11 | 12 | R95T | | |
| IL4-0157 | 18 | N60S, P61T, S65T (65) | 3 | 21 | 28 | | 24 | 12 | N92H, E93K, R95T (68) | 29 | |
| IL4-0751 | 18 | 2 | 3 | 21 | 22 | 23 | Y27dE | 12 | 68 | | |
| IL4-0752 | 18 | 2 | 3 | 21 | 22 | 23 | Y27dE | 12 | 76 | | |
| IL4-0753 | 18 | 2 | 3 | 21 | 22 | 23 | Y27dE | 12 | 25 | | |
| IL4-1345 | 1 | 2 | 3 | 4 | 5 | 10 | Y27dN | 12 | R95T | | |
| IL4-1346 | 1 | 2 | 3 | 4 | 5 | 10 | Y27dF | 12 | R95T | | |
| IL4-1347 | 1 | 2 | 3 | 4 | 5 | 10 | Y27dE | 12 | R95T | 20 | |
| IL4-1348 | 1 | 2 | 3 | 4 | 5 | 10 | D28E | 12 | R95T | | |
| IL4-1349 | S32W M34S | 2 | 3 | 4 | | | Y27dF | 12 | R95T | | |
| IL4-1350 | S32W M34S | 2 | 3 | 4 | | | Y27dE | 12 | R95T | 20 | |
| IL4-1351 | T31N S32W | 2 | 3 | 4 | | | Y27dF | 12 | R95T | | |
| IL4-1352 | T3AN S32W | 2 | 3 | 4 | | | Y27dE | | R95T | 20 | |
| IL4-1353 | S30L T31A S32W M34E | 2 | 3 | 4 | | | Y27dF | 12 | R95T | | |
| | V35al | | | | | | | | | | |
| IL4-1354 | S30L T31A S32W M34E | 2 | 3 | 4 | | | Y27dE | 12 | R95T | 20 | |
| | V35al | | | | | | | | | | |
| IL4-1355 | S30L S32F M345 | 2 | 3 | 4 | | | Y27dF | 12 | R95T | | |
| IL4-1356 | S30L S32F M345 | 2 | 3 | 4 | | | Y27dE | 12 | R95T | 20 | |
| IL4-1357 | S32W M34E | 2 | 3 | 4 | | | Y27dF | 12 | R95T | | |
| IL4-1358 | S32W M34E | 2 | 3 | 4 | | | Y27dE | 12 | R95T | 20 | |
| IL4-1322 | 18 | 2 | 3 | 4 | | | Y27dF | 12 | R95T | | |
| IL4-1359 | 18 | 2 | 3 | 4 | | | Y27dE | 12 | R95T | 20 | |
| IL4-0037 | 18 | N60S, P61T, S65T | | 21 | 28 | | Y27dE | 12 | R95T | 20 | |
| IL4-0092 | 18 | 2 | | 21 | 22 | 23 | K24R Y27dE | 12 | R95T | | |
| IL4-0093 | 18 | N60S, P61T, S65T | | 21 | 28 | | K24R Y27dE | 12 | R95T | | |
| IL4-0749 | 18 | 2 | 3 | 21 | 22 | 23 | 24 | 12 | R95T | 30 | 208 |
| IL4-0754 | 18 | 2 | 3 | 21 | 3 | 23 | K24R Y27dE | 12 | R95T | | |

TABLE 81
IL-13 antibodies. IL13-1306 is murine; all other Abs are humanized with the following; CH1 is SEQ ID NO: 6; HINGE is SEQ
ID NO: 7; CH2 is SEQ ID NO: 8, CH3 is SEQ ID NO: 9. IL13-0259 and IL13-0271 comprise a JH of SEQ ID NO: 56. All other humanized
Abs have JH of SEQ ID NO: 47. JK is SEQ ID NO: 14: CL is SEQ ID NO: 16. Mutations are with reference to benchmark antibody IMA-638 (IL 13-1283).

| IL-13 | | | | | | | | | | |
|---|---|---|---|---|---|---|---|---|---|---|
| Antibody | CDR H1 | CDR H2 | CDR H3 | V_{H} | HC | CDR L1 | CDR L2 | CDR L3 | V_{L} | LC |
| IL13-1283 | GFTFISYAMS | SISSGGNTYYPDSVKG | LDGYYFGFAY (33) | 38 | 35 | KASESVDNYGKSLMH (38) | RASNLES | 38 | 39 | 40 |
| | (31) | (32) | | | | | (37) | | | |
| IL13-1306 | GFTFSSYAMS | SISSGDTTYYPDSVKG | LDGYYFGFPY (43) | 44 | | KASESYDHSGSSLMH (45) | 37 | 38 | 46 | |
| | (41) | (42) | | | | | | | | |
| IL13-1307 | 41 | 42 | 13 | 48 | | 45 | 37 | 38 | 49 | |
| | | | | | | | 37 | 38 | | |
| IL13-0001 | 41 | SISSGDTTYYPDSVKG | NEGYYFGLTL (50) | 51 | 52 | KASESVDHFGWSLVH (53) | 37 | 38 | 54 | 55 |
| | | (42) | | | | | | | | |
| IL13-1308 | 41 | 42 | 43 | 48 | | 53 | 37 | 38 | 68 | 55 |
| IL13-1309 | 41 | 42 | 43 | 48 | | N27dH, Y28A, K30F | 37 | 38 | | |
| IL13-1310 | 41 | 42 | 43 | 48 | | N27dH, Y28N, K30W M33L | 37 | 38 | | |
| IL13-1311 | 41 | 42 | 43 | 48 | | N27dH, Y28S, G29T, K30F, M33L | 37 | 38 | | |
| IL13-1312 | 41 | 42 | 43 | 48 | | N27dW, Y28S, K30F, M33L | 37 | 38 | | |
| IL13-1313 | 41 | 42 | 43 | 48 | | N27dH, Y28S, K30W | 37 | 38 | | |
| IL13-1314 | 41 | 42 | 43 | 48 | | N27dH, Y28N, K30F, M33I | 37 | 38 | | |
| IL13-1315 | 41 | 42 | 43 | 48 | | N27dH, Y28G, G29A, K30F | 37 | 38 | | |
| IL13-1316 | 41 | 42 | 43 | 48 | | N27dY, Y28R, K30F, M33I | 37 | 38 | | |
| IL13-1317 | 41 | 42 | 43 | 48 | | N27dH, Y28F, K30F, M33L | 37 | 38 | | |
| IL13-1318 | 41 | 42 | 43 | 48 | | E27K, D27cS, N27dH, Y28S, K30F, | 37 | 38 | | |
| | | | | | | M33L | | | | |
| IL13-1319 | 41 | 42 | 43 | 48 | | E27K, N27dH, K30H | 37 | 38 | | |
| IL13-1320 | 41 | 42 | 43 | 48 | | A25S, E27K, D27cS, N27dH, K30H, | 37 | 38 | | |
| | | | | | | S31N | | | | |
| IL13-1321 | 41 | 42 | 43 | 48 | | A25S, E27K, N27dH, K30H | 37 | 38 | | |
| IL13-1323 | 41 | 42 | 43 | 48 | | E27Q, N27dH, S31N M33L | 37 | 38 | | |
| IL13-1324 | 41 | 42 | 43 | 48 | | A25S, N27dH, Y28S. K30H, M33L | 37 | 38 | | |
| IL13-1325 | 41 | 42 | 43 | 48 | | E27Q, D27cS, N27dH, S31N | 37 | 38 | | |
| IL13-1326 | 41 | 42 | 43 | 48 | | E27K, D27cS, N27dH, G29S, K30H, | 37 | 38 | | |
| | | | | | | S31N, M33L | | | | |
| IL13-1327 | 41 | 42 | 43 | 48 | | E27Q, N27dH, K30H | 37 | 36 | | |
| IL13-1328 | 41 | 42 | 43 | 48 | | A25S, E27K, D27cS, N27dH, G29S | 37 | 38 | | |
| | | | | | | K30H, M33L | | | | |
| IL13-1329 | I30V | 42 | 43 | | | 45 | 37 | 30 | 49 | |
| IL13-1330 | I30A | 42 | 43 | | | 45 | 37 | 38 | 49 | |
| IL13-1331 | T28P, I29A, | 42 | 43 | | | 45 | 37 | 38 | 49 | |
| | S30A | | | | | | | | | |
| IL13-1332 | 41 | S53V G54V N56T | 43 | | | 45 | 37 | 38 | 49 | |
| IL13-1333 | 41 | S53V, G55D, N56L, | 43 | | | 45 | 37 | 38 | 49 | |
| | | T57R | | | | | | | | |
| IL13-1334 | 41 | G55Y N56I | 43 | | | 45 | 37 | 38 | 49 | |
| IL13-1335 | 41 | G54L N56Q | 43 | | | 45 | 37 | 38 | 49 | |
| IL13-1336 | 41 | G54L N56T | 43 | | | 45 | 37 | 38 | 49 | |
| IL13-1337 | 41 | 42 | L95N D96E A103S | | | 45 | 37 | 38 | 49 | |
| | | | Y102L | | | | | | | |
| IL13-1338 | | 42 | 50 | 65 | 66 | 45 | 37 | 38 | 49 | |
| IL13-1339 | 41 | 42 | L95N D96E F100bH | | | 45 | 37 | 38 | 49 | |
| | | | A101T Y102L | | | | | | | |
| IL13-1340 | 41 | 42 | L95N D96S F100bL | | | 45 | 37 | 38 | 19 | |
| | | | A101S Y102L | | | | | | | |
| IL13-1341 | 41 | 42 | L95N D96S F100Y, | | | 45 | 37 | 38 | 49 | |
| | | | F101bL, A101S, | | | | | | | |
| | | | Y102L | | | | | | | |
| IL13-1342 | 41 | 42 | L95N D96S A101S | | | 45 | 37 | 38 | 49 | |
| | | | Y102L | | | | | | | |
| IL13-1343 | 41 | 42 | L95N D96E A101T | | | 45 | 37 | 38 | 49 | |
| | | | Y102L | | | | | | | |
| IL13-1344 | 41 | 42 | L95N D96E F101bL | | | 45 | 37 | 38 | 49 | |
| | | | A101T Y102H | | | | | | | |
| IL13-0259 | 41 | S53T G55D N56T | L95N D96E F101bL | 57 | | K24R, E27Q, N27dH, Y28F, K30W, | 37 | 38 | 58 | |
| | | | A101T Y182Q | | | M33V | | | | |
| IL13-0270 | 41 | S53T G55D N56T | L85N D96E F101bl | | | K24R, N27dH, Y28F, K30W, M33V | S52D N53S | 38 | 59 | |
| | | | A101T Y102L | | | | S56T | | | |
| IL13-0271 | 41 | S53T G55D N56T | L95N D96E F101bL | 57 | | K24R, N27dH, Y28F, K30W, M33V | S52D N53S | | 59 | |
| | | | A101T Y1820 | | | | S56T | | | |

TABLE 82
IL-33 antibodies. JH is SEQ ID NO: 111; CH1 is SEQ ID NO: 6; HINGE is SEQ ID NO: 7; CH2 is SEQ ID NO: 8; CH3 is
SEQ ID NO: 9; JK is SEQ ID NO: 14; CL is SEQ ID NO: 16. HC of IL33-0232 is SEQ ID NO: 251. Reference antibody is IL33-0232.

| IL-33 | | | | | | | | | | |
|---|---|---|---|---|---|---|---|---|---|---|
| Antibody | CDR H1 | CDR H2 | CDR H3 | V_{H} | HC | CDR L1 | CDR L2 | CDR L3 | V_{L} | LC |
| IL33- | SFTFSSYWMY | AITPNAGEDYYPESVKG | GHYYYTSYSLGY | 63 | 64 | KASQNINKHLD | FTMNLQT | FQYNQGWT (67) | 68 | 69 |
| 0232 | (60) | (61) | (62) | | | (65) | (66) | | | |
| IL33- | 60 | 61 | 62 | 63 | 64 | RASQPISKHLD | 66 | 67 | 71 | |
| 0224 | | | | | | (70) | | | | |
| IL33- | 60 | 61 | GQYYYTKYSLGY | 73 | 74 | RASQPIHNHLD | EGKMLQF | FQYKKGWS (77) | 78 | 79 |
| 0726 | | | (72) | | | (75) | (76) | | | |
| IL33- | 60 | G55H, E56A, D57G | 62 | | | 70 | 66 | N92K | | |
| 0698 | | | | | | | | | | |
| IL33- | 60 | A54K, E56V | 62 | | | 70 | 66 | N92K | | |
| 0701 | | | | | | | | | | |
| IL33- | 60 | A54K, E56V | 62 | | | 70 | N52S, Q55Y | N92K | | |
| 0702 | | | | | | | | | | |
| IL33- | 60 | V63T | Y98F, S100cR | | | K24R, Q27K, | N52T, T56I | N92K | | |
| 0704 | | | | | | N28P, N30S | | | | |
| IL33- | F27L | T52P, N53K | Y98F, S100cR | | | (119) | N52T, T56I | N92K | | |
| 0705 | | | | | | | | | | |
| IL33- | F27L | T52P, N53K | Y98F, S100cR | | | K24R, Q27K, | 66 | F89Y, N92S, | | |
| 0706 | | | | | | N28P, N30S | | | | |
| IL33- | F27L | A54K, E56V | Y98F, S100cR | | | 70 | N527, T56I | N92K | | |
| 0707 | | | | | | | | | | |
| IL33- | 60 | G55H, E56A, D57G | 62 | | | 70 | N52K, Q55V | 67 | | |
| 0710 | | | | | | | | | | |
| IL33- | 60 | G55H, E56A, D57G | 62 | | | 70 | N52K, Q55K | 67 | | |
| 0711 | | | | | | | | | | |
| IL33- | 66 | G55H, E56A, D57G | 62 | | | 70 | N52K, T56S | 67 | | |
| 0712 | | | | | | | | | | |
| IL33- | 60 | A54K, E56V | 62 | | | 70 | N52K, Q55V | 67 | | |
| 0713 | | | | | | | | | | |
| IL33- | 60 | A54K, E56V | 62 | | | 70 | N52K, Q55K | 67 | | |
| 0714 | | | | | | | | | | |
| IL33- | 60 | A54K, E56V | 62 | | | 70 | N52K, T56S | 67 | | |
| 0715 | | | | | | | | | | |
| IL33- | 60 | 61 | Y98Y, T100V, | | | K24R, N28P, | T51G, N52K, | F89Q, N92F, Q93R, | | |
| 0718 | | | | | | N30R, K31D | N53R, T56E | T96V | | |
| IL33- | 60 | 61 | Y98R. T100V, | | | K24R, N28P, | N52K, T56E | QQYRQGWTFGGGT | | |
| 0719 | | | | | | N30Q, K31Q | | | | |
| IL33- | T28E, S31H | 61 | H96Y, R98, | 80 | | K24R, N28P, | T51G, N52H, | N92K, T96S | 81 | |
| 0721 | | | S100aK | | | N30H, K31Q, | N53I, T56E | | | |
| | | | | | | H32F | | | | |
| IL33- | T28P, S31N | A50G, T52S, P52aH. | Y99F, T100N, | | | K24R, N28P, | T51G, N53K, | N92S, Q538R | | |
| 0722 | | N53W | S100aK | | | N30Q, K310 | T56E | | | |
| IL33- | T28E, S31N | P52aH | H96F, Y98R, | | | 75 | T51G, N52H, | N92W, Q93K | | |
| 0723 | | | T100N, S100aK | | | | N53Q | | | |
| IL33- | 60 | T52S, N53I, E56D | Y98R, T100V, | | | K24R, N28P, | T51G, N52R, | F89Q, N92K, Q93H | | |
| 0724 | | | S100a0 | | | N30R, K31D | N53H, T56E | | | |
| IL33- | T28S | T52S, P26aY, E66N | Y99F, T100N, | | | K24R, N28P, | T51G, N52R, | N92H, G94K, | | |
| 0727 | | | S100aA | | | N30H, K31Q | T56E | T96S | | |
| IL33- | T28E, S31N | T52S, P52aY, E56N | H96Y, Y98R, | | | K24R, N28P, | T51G, N52R, | N92Y, Q93K, T96V | | |
| 0728 | | | T100N, S100aR | | | N30Q, K31Q | Q55E | | | |
| IL33- | T28P, S31N | N53Y, E56H, | H96Y, Y98R, | | | RASQPIQEHLD | T51G, N52R, | | | |
| 0737 | | | T100V, S100aQ | | | | N53H, T56E | | | |
| IL33- | T28P, S31N | T52S, P52aH, E56Q | H96Q, Y99F, | | | K24R, N28P, | T51G, N52H, | F89Q, N92R, Q93H | | |
| 0738 | | | | | | N30H, K31Q | N53R | | | |
| IL33- | T28P | T52S, E56H | Y98R, T100V, | | | K24R, N28P, | T51G, N52K, | F89Q, N92F, Q93R | | |
| 0739 | | | S100aQ | | | N30R, K31E | N53K, Q55E, | | | |
| | | | | | | | T56E | | | |
| IL33- | T28P, S31Q | A50G, T52S, P52aH, | Y98R, T100N, | | | 75 | T51G, N52H, | F89Q, N92F, Q93K, | | |
| 0740 | | N53Y, E66Q | S100aA | | | | N53I, T66E | T96S | | |
| IL33- | T28R, S31N | 71 | H96Y, Y99W, | | | K24R, N28P, | N52K, N53R, | F89Q, N92Q, Q93K | | |
| 0741 | | | T100V, S100aK | | | N30Y, K31N | T56E | | | |

TABLE 83
TSLP Antibodies. JH is SEQ ID NO: 100; CH1 is SEQ ID NO: 6; HINGE is SEQ ID NO: 7; CH2 is SEQ
ID NO: 8; CH3 is SEQ ID NO: 9; Jλ iS SEQ ID NO: 101; CL is SEQ ID NO: 95. Mutations are
compared to reference antibody TSLP-0001.

| TSLP | | | | | | | | | | |
|---|---|---|---|---|---|---|---|---|---|---|
| Antibody | CDR H1 | GDR H2 | CdR H3 | V_{H} | HC | CDR L1 | CDR L2 | CDR L3 | V_{L} | LC |
| TSLP- | GFTFRT | VIWYDGSNKHY | APQWELVHEAFDI | 91 | 96 | GGNNLGSKSVH | DDSDRPS | QVWDSSSDHVV | 93 | 98 |
| 0001 | YGMH | ADSVKG | (84) | | | (86) | (87) | (89) | | |
| | (82) | (83) | | | | | | | | |
| TSLP- | 82 | 83 | (84) | 210 | | 86 | 87 | 89 | 93 | 98 |
| 0100 | | | | | | | | | | |
| TSLP- | 82 | 83 | APQWYLVHEAFDI | 92 | 97 | 86 | 87 | 89 | 93 | 98 |
| 0156 | | | (85) | | | | | | | |
| TSLP- | 82 | 83 | 85 | 92 | 97 | 86 | 87 | QVWDESSDHVV | | |
| 0260 | | | | | | | | | | |
| TSEP- | 82 | 83 | 85 | 221 | 222 | 86 | 87 | 89 | 93 | 98 |
| 0820 | | | | | | | | | | |
| TSLP- | 82 | 83 | 85 | | | 86 | 87 | 89 | 93 | 98 |
| 0821 | | | | | | | | | | |
| TSLP- | 82 | 83 | 85 | 92 | 97 | 86 | 87 | QVWDSSSKRVV | 213 | 215 |
| 0855 | | | | | | | | (211) | | |
| TSLP- | 82 | 83 | 85 | 92 | 97 | 86 | DDKDRPS | QVWDKSSDHVV | 214 | 216 |
| 0871 | | | | | | | (88) | (212) | | |
| TSLP- | GFTFRT | VIWYDGSNKHY | APQWYLVHEAFDI | 92 | 97 | 86 | DDKDRPS | QVWDSKSDHVV | 94 | 99 |
| 0875 | YGMH | ADSVKG | (85) | | | | (88) | (90) | | |
| | (82) | (83) | | | | | | | | |
| TSLP- | 82 | 83 | 85 | 221 | 222 | 86 | 87 | 211 | 213 | 215 |
| 2000 | | | | | | | | | | |
| TSLP- | 82 | 83 | 85 | 221 | 222 | 86 | 88 | 90 | 94 | 99 |
| 2002 | | | | | | | | | | |
| TSLP- | 82 | 83 | 85 | 221 | 222 | 86 | 87 | 212 | 223 | 224 |
| 2004 | | | | | | | | | | |

[symbol]

TABLE 84
IL4IL13TSLP Multispecific antibodies. SEQ ID numbers for relevant fragments and domains, VH [ ] parenthesis.

| Variable | TSLP CH- | TSLP CH- | TSLP CH- | | | | TSLP no | TSLP no |
|---|---|---|---|---|---|---|---|---|
| domain | CL swap | CL swap | CL swap | IL13 CH-CL | TSLP VH-VL | TSLP VH-VL | swap (native | swap (native |
| format | (CkS) | (CkS) | (CkS) | swap (CkS) | swap (VDS) | swap (VDS) | Fab) | Fab) |
| Ch or CL | KiH | KiH | KiH | RRR-EEE | KiH | KiH | KiH | KiH |
| engineering | triFab-Fc | | | | triFab-Fc | | triFab-Fc | |
| Domain | IL413TSLP- | IL413TSLP- | IL413TSLP- | IL413TSLP- | IL413TSLP- | IL413TSLP- | IL413TSLP- | IL413TSLP- |
| | 0001 | 0002 | 0249 | 0251 | 0007 | 0008 | 0003 | 0004 |
| | TSLP- | TSLP- | TSLP- | IL13- | TSLP- | TSLP- | TSLP- | TSLP- |
| | 0001/VL | 0001/VL | 1000/VL | 0001/VL | 0001/VL | 0001/VL | 0001/VL | 0001/VL |
| [symbol] | 93 | 93 | 93 | 54 | [91] | [91] | 93 | 93 |
| | CHI (1) SEQ ID NO: 6 | | | | CL(1) SEQ ID NO: 6 | | CL(1) SEQ ID NO: 6 | |
| Upper | 102 | 102 | 102 | 102 | — | — | — | — |
| HINGE(1) | | | | | | | | |
| | VL-CH1 (1) | | | | VL-CH1 (1) | | VL-CH1 (1) | |
| SEQ | 150 | 150 | 150 | 163 | 155 | 155 | 98 | 98 |
| VH (1) | TSLP- | TSLP- | TSLP- | IL13- | TSLP- | TSLP- | TSLP- | TSLP- |
| | 0001/VH | 0001/VH | 0100/VH | 0001/VH | 0001/VH | 0001/VH | 0001/VH | 0001/VH |
| VL(1)/[VH(1)] | [91] | [91] | [210] | [51] | 93 | 93 | [91] | [91] |
| CL(1) | 95 | 95 | 95 | 16 | [6] | [6] | [6] | [6] |
| CH1(1) | | | | | | | | |
| Upper | — | — | — | — | 260 | 260 | 260 | 260 |
| HINGE(1) | | | | | | | | |
| LINKER | 104 | 104 | 104 | 104 | 104 | 104 | 104 | 104 |
| | IL13- | IL4- | IL4- | IL4- | IL13- | IL4- | IL13- | IL4- |
| | 0001/VH | 0002/VH | 0002/VH | 0002/VH | 0001/VH | 1040/VH | 0001/VH | 0002/VH |
| MH(2) | [51] | [22] | [22] | [22] | [51] | [22] | [51] | [22] |
| CH1(2) | 110 | 105 | 105 | 6 | 110 | 105 | 110 | 105 |
| HINGE(2) | 7 | 7 | 7 | 126 | 7 | 7 | 7 | 7 |
| CH2(2) | 8 | 8 | 8 | 8 | 8 | 8 | 8 | 8 |
| CH3(2) | 111 | 106 | 106 | 148 | 111 | 106 | 111 | 106 |
| DFab SEQ | DFab VH-CL(1)-HC(2) | | | | DFab VH-CL(1)-HC(2) | | DFab VH-CL(1)-HC(2) | |
| | 149 | 151 | 159 | 162 | 154 | 156 | 152 | 153 |
| | IL13- | IL4- | IL4- | IL4- | IL13- | IL4- | IL13- | IL4- |
| | 0001/VH | 0002/VH | 0002/VH | 0002/VH | 0001/VH | 0002/VH | 0001/VH | 0002/VH |
| VL(2) | 54 | 20 | 20 | 20 | 54 | 20 | 54 | 20 |
| CL(2) | 113 | 108 | 108 | 16 | 143 | 108 | 113 | 108 |
| DFab LC(2) | 196 | 109 | 109 | 197 | 196 | 109 | 106 | 109 |
| VH (3) | IL4- | IL13- | IL13- | TSLP- | IL4- | IL13- | IL4- | IL13- |
| | 0002/VH | 0001/VH | 0001/VH | 0100/VH | 0002/VH | 0001/VH | 1040/VH | 0001/VH |
| VH(3) | [22] | [51] | [51] | [210] | [22] | [51] | [22] | [51] |
| CH1(3) | 106 | 110 | 110 | 6 | 105 | 110 | 105 | 110 |
| SFab HC | 7 | 7 | 7 | 126 | 7 | 7 | 7 | 7 |
| HINGE(3) | | | | | | | | |
| CH2(3) | 8 | 8 | 8 | 8 | 8 | 8 | 8 | 8 |
| CH3(3) | 146 | 112 | 112 | 161 | 146 | 112 | 146 | 112 |
| VL (3) | IL4- | IL13- | IL13- | TSLP- | IL4- | IL13- | IL4- | IL13- |
| | 0002/VH | 0001/VH | 0001/VH | 0100/VH | 0002/VH | 0001/VH | 0002/VH | 0001/VH |
| VL(3) | 20 | 54 | 54 | 93 | 20 | 54 | 20 | 54 |
| CL(3) | 108 | 113 | 113 | 95 | 108 | 113 | 108 | 113 |
| Sfab LC(3) | 109 | 196 | 196 | 98 | 109 | 196 | 109 | 196 |
| Variable | | | | | | | | |
| domain | TSLP | TSLP | | | | | | |
| format | mEd | mEd | IL13 mEd | IL13 mEd | IL13 mFd | IL13 mFd | | |
| Ch or CL | KiH | KiH | RRR-EEE | RR-EE-LS | RR-EE, LS | RR-EE, LS | | |
| engineering | triFab-Fc | | | | triFab-Fc | | | |
| Domain | IL413TSLP- | IL413TSLP- | IL413TSLP- | IL413TSLP- | IL413TSLP-1028 | IL413TSLP-1037 | | |
| | 0248 | 0250 | 0252 | 1024 | IL13-0001/VH | IL13-0001/VH | | |
| | TSLP- | TSLP- | IL13- | IL13- | | | | |
| | 0100/VH | 0100/VH | 0001/VH | 0001/VH | | | | |
| VL(1)/[VH(1)] | [210] | [210] | [51] | [51] | [51] | [51] | | |
| | CHI (1) SEQ ID NO: 6 | | | | CH1 (1) SEQ ID NO: 6 | | | |
| Upper | 102 | 102 | 102 | 102 | 102 | 102 | | |
| HINGE(1) | | | | | | | | |
| | [symbol] Modified (1) | | | | Fd Modified (1) | | | |
| SEQ | 158 | 158 | 122 | 122 | 122 | 122 | | |
| VH (1) | TSLP- | TSLP- | IL13- | IL13- | IL13-0001/VL | IL13-0001/VL | | |
| | 0100/VH | 0100/VH | 0001/VH | 0001/VH | | | | |
| VL (1) [VH(1)] | 93 | 93 | 64 | 64 | 54 | 54 | | |
| CL(1) | 95 | 95 | 16 | 16 | 16 | 16 | | |
| CH1(1) | | | | | | | | |
| Upper | — | — | — | — | — | — | | |
| HINGE(1) | | | | | | | | |
| LINKER | 104 | 104 | 104 | 104 | 104 | 104 | | |
| | IL4- | IL13- | IL4- | IL4- | IL4-1040/VH | IL4-1040/VH | | |
| | 0002/VH | 0001/VH | 0002/VH | 1040/VH | | | | |
| MH(2) | [22] | [51] | [22] | [22] | [22] | [22] | | |
| CH1(2) | 105 | 110 | 6 | 6 | 6 | 6 | | |
| HINGE(2) | 7 | 7 | 123 | 123 | 129 | 129 | | |
| CH2(2) | 8 | 8 | 8 | 8 | 8 | 8 | | |
| CH3(2) | 106 | 111 | 146 | 124 | 124 | 124 | | |
| DFab SEQ | DFab CL(1)-HC(2) | | | | DFab LC(1)-HC(2) | | | |
| | 157 | 100 | 164 | 130 | 130 | 130 | | |
| | IL4- | IL13- | IL4- | IL4- | IL4-1040/VL | IL4-1040/VL | | |
| | 0002/VH | 0001/VH | 0002/VH | 1040/VH | | | | |
| VL(2) | 20 | 54 | 20 | 26 | 26 | 26 | | |
| CL(2) | 108 | 113 | 16 | 16 | 16 | 16 | | |
| DFab LC(2) | 109 | 196 | 197 | 27 | 27 | 27 | | |
| VH (3) | IL13- | IL4- | TSLP- | TSLP- | TSLP-0855/VH | TSLP-0871/VH | | |
| | 0001/VH | 1040/VH | 0100/VH | 0875/VH | | | | |
| VH(3) | [51] | [22] | [210] | [92] | [92] | [92] | | |
| CH1(3) | 110 | 105 | 6 | 6 | 6 | 6 | | |
| SFab HC | 7 | 7 | 126 | 131 | 131 | 131 | | |
| HINGE(3) | | | | | | | | |
| CH2(3) | 8 | 8 | 8 | 8 | 8 | 8 | | |
| CH3(3) | 112 | 146 | 161 | 165 | 127 | 127 | | |
| VL (3) | IL13- | IL4- | TSLP- | TSLP- | 165 | 165 | | |
| | 0001/VH | 0002/VH | 0100/VH | 0875/VH | TSLP-0855/VL | TSLP-0871/VL | | |
| VL(3) | 54 | 20 | 93 | 94 | 213 | 214 | | |
| CL(3) | 113 | 108 | 95 | 95 | 95 | 95 | | |
| Sfab LC(3) | 196 | 108 | 98 | 99 | 215 | 216 | | |

[symbol]

indicates data missing or illegible when filed

[symbol]

TABLE 85
IL4IL13IL33 Multispecific antibodies. SEQ ID numbers for relevant fragments and domains. JH1 SEQ ID NO: 111.

| | Tri-Fab-Fc | | | | | | | | | | |
|---|---|---|---|---|---|---|---|---|---|---|---|
| | IL1343 | IL1343 | IL1343 | IL1343 | IL1343 | IL1343 | IL1343 | IL1343 | IL1343 | IL1343 | IL1343 |
| Domain | 3-0005 | 3-0006 | 3-0606 | 3-0607 | 3-0717 | 3-1042 | 3-1258 | 3-1261 | 3-1269 | 3-1270 | 3-1275 |
| VH(1) | IL33- | IL33- | IL33- | IL33- | IL33- | IL13- | IL13- | IL13- | IL33- | IL13- | IL13- |
| | 0232 | 0232 | 0224 | 0224 | 0232 | 0001 | 0001 | 0001 | 0726 | 0001 | 0001 |
| V_{H}(1) | 63 | 63 | 63 | 63 | 63 | 51 | 51 | 51 | 73 | 51 | 51 |
| CH1(1) | 6 | 6 | 6 | 6 | 6 | 6 | 6 | 6 | 6 | 6 | 6 |
| Upper | 102 | 102 | 102 | 102 | 102 | 102 | 102 | 102 | 102 | 102 | 102 |
| HINGE(1) | | | | | | | | | | | |
| Modified | 103 | 103 | 103 | 103 | 103 | 122 | 122 | 122 | 143 | 122 | 122 |
| Fd(1) | | | | | | | | | | | |
| VL (1) | IL33- | IL33- | IL33- | IL33- | IL33- | IL13- | IL13- | IL13- | IL33- | IL13- | IL13- |
| | 0232 | 0232 | 0224 | 0224 | 0232 | 0001 | 0001 | 0001 | 0726 | 0001 | 0001 |
| V_{L}(1) | 68 | 68 | 71 | 71 | 68 | 54 | 54 | 54 | 78 | 54 | 54 |
| CL(1) | 16 | 16 | 16 | 16 | 16 | 16 | 16 | 16 | 16 | 16 | 16 |
| LINKER | 104 | 104 | 104 | 104 | 104 | 104 | 104 | 104 | 104 | 104 | 104 |
| VH(2) | IL13- | IL4- | IL4- | IL4- | IL4- | IL4- | IL4- | IL4- | IL4- | IL4- | IL4- |
| | 0001 | 0002 | 0157 | 0157 | 0002 | 0749 | 1040 | 1040 | 1040 | 1040 | 1040 |
| V_{H}(2) | 51 | 22 | 28 | 28 | 22 | 22 | 22 | 22 | 22 | 22 | 22 |
| CH1(2) | 110 | 105 | 105 | 105 | 105 | 6 | 6 | 6 | 105 | 105 | 6 |
| HINGE(2) | 7 | 7 | 7 | 7 | 7 | 123 | 129 | 7 | 7 | 7 | 7 |
| CH2(2) | 8 | 8 | 8 | 8 | 8 | 8 | 8 | 8 | 8 | 8 | 8 |
| CH3(2) | 111 | 106 | 114 | 114 | 114 | 124 | 124 | 124 | 114 | 114 | 139 |
| DFab | 145 | 107 | 115 | 115 | 121 | 125 | 130 | 133 | 144 | 135 | 140 |
| LC(1)- | | | | | | | | | | | |
| HC(2) | | | | | | | | | | | |
| LC (2) | IL13- | IL4- | IL4- | IL4- | IL4- | IL4- | IL4- | IL4- | IL4- | IL4- | IL4- |
| | 0001 | 0002 | 0157 | 0157 | 0002 | 0749 | 1040 | 1040 | 1040 | 1040 | 1040 |
| V_{L}(2) | 54 | 20 | 29 | 29 | 20 | 30 | 26 | 26 | 26 | 26 | 26 |
| CL(2) | 113 | 108 | 108 | 108 | 108 | 16 | 16 | 16 | 108 | 108 | 16 |
| DFab | 196 | 109 | 116 | 116 | 109 | 208 | 27 | 27 | 136 | 136 | 27 |
| LC(2) | | | | | | | | | | | |
| HC(3) | IL4- | IL13- | IL13- | IL13- | IL13- | IL33- | IL33- | IL33- | IL13- | IL33- | IL33- |
| | 0002 | 0001 | 0259 | 0271 | 0001 | 0726 | 0726 | 0726 | 0001 | 0726 | 0726 |
| V_{H}(3) | 22 | 51 | 57 | 57 | 51 | 73 | 73 | 73 | 51 | 73 | 73 |
| CH1(3) | 105 | 110 | 110 | 110 | 110 | 6 | 6 | 6 | 110 | 110 | 6 |
| SFab | 7 | 7 | 7 | 7 | 7 | 126 | 131 | 7 | 7 | 7 | 7 |
| HC | | | | | | | | | | | |
| HINGE(3) | | | | | | | | | | | |
| CH2(3) | 8 | 8 | 8 | 8 | 8 | 8 | 8 | 8 | 8 | 8 | 8 |
| CH3 (3) | 106 | 111 | 117 | 117 | 117 | 127 | 127 | 127 | 117 | 117 | 141 |
| SFab | 146 | 112 | 118 | 118 | 209 | 128 | 132 | 134 | 121 | 137 | 142 |
| HC(3) | | | | | | | | | | | |
| LC (3) | IL4- | IL13- | IL13- | IL13- | IL13- | IL33- | IL33- | IL33- | IL13- | IL33- | IL33- |
| | 0002 | 0001 | 0259 | 0271 | 0001 | 0726 | 0726 | 0726 | 0001 | 0726 | 0726 |
| V_{L}(3) | 20 | 122 | 58 | 59 | 122 | 78 | 78 | 78 | 122 | 78 | 78 |
| CL(3) | 108 | 113 | 113 | 113 | 113 | 16 | 16 | 16 | 113 | 113 | 16 |
| SFab | 109 | 196 | 119 | 120 | 196 | 79 | 79 | 79 | 196 | 138 | 79 |
| LC(3) | | | | | | | | | | | |

TABLE 86
IL4IL13p40 Multispecific antibodies. SEQ ID numbers for relevant fragments and domains.

| | Tri-Fab-Fc | | | | | | |
|---|---|---|---|---|---|---|---|
| | IL413p40- | IL413p40- | IL413p40- | IL13433- | IL413p40- | IL413p40- | IL413p40- |
| Domain | 0043 | 0044 | 0642 | 0648 | 0698 | 0700 | 0705 |
| VH(1) | P40- | P40- | IL13- | P40- | IL13- | IL13- | IL13- |
| | 0003/VH | 0003/VH | 0001/VH | 0003/VH | 0001/VH | 0001/VH | 0001/VH |
| V_{H}(1) | 169 | 169 | 51 | 169 | 51 | 51 | 51 |
| CH1(1) | 6 | 6 | 6 | 6 | 6 | 6 | 6 |
| Upper | 102 | 102 | 102 | 102 | 102 | 102 | 102 |
| HINGE(1) | | | | | | | |
| Modified | 177 | 177 | 122 | 177 | 122 | 122 | 122 |
| Fd(1) | | | | | | | |
| VL(1) | P40- | P40- | IL13- | P40- | IL13- | IL13- | IL13- |
| | 0003/VL | 0003/VL | 0001/VL | 0003/VL | 0001/VL | 0001/VL | 0001/VL |
| V_{L}(1) | 175 | 175 | 54 | 175 | 54 | 54 | 54 |
| CL(1) | 16 | 16 | 16 | 16 | 16 | 16 | 16 |
| LINKER | 104 | 104 | 104 | 104 | 104 | 104 | 104 |
| VH(2) | IL13- | IL4-0002/VH | IL4-0002/VH | IL4- | IL4-0749/VH | IL4-1040/VH | IL4-1040/VH |
| | 0001/VH | | | 0157/VH | | | |
| V_{H}(2) | 51 | 22 | 22 | 28 | 22 | 22 | 22 |
| CH1(2) | 110 | 105 | 105 | 105 | 6 | 6 | 6 |
| HINGE(2) | 7 | 7 | 7 | 7 | 123 | 123 | 129 |
| CH2(2) | 8 | 8 | 8 | 8 | 8 | 8 | 8 |
| CH3(2) | 111 | 106 | 114 | 114 | 124 | 124 | 124 |
| DFab | 178 | 179 | 180 | 183 | 125 | 125 | 130 |
| LC(1)- | | | | | | | |
| HC(2) | | | | | | | |
| VL(2) | IL13- | IL4-0002/VL | IL4-0002/VL | IL4- | IL4-0749/VL | IL4-1040/VL | IL4-1040/VL |
| | 0001/VL | | | 0157/VL | | | |
| V_{L}(2) | 54 | 20 | 20 | 29 | 30 | 26 | 26 |
| CL(2) | 113 | 108 | 108 | 108 | 16 | 16 | 16 |
| DFab | 196 | 109 | 109 | 116 | 208 | 27 | 27 |
| LC(2) | | | | | | | |
| VH(3) | IL4-0002/VH | IL13- | P40- | IL13- | P40- | P40- | P40- |
| | | 0001/VH | 0003/VH | 0271/VH | 0003/VH | 0003/VH | 0003/VH |
| V_{H}(3) | 22 | 51 | 169 | 57 | 169 | 169 | 169 |
| CH1(3) | 105 | 110 | 110 | 110 | 6 | 6 | 6 |
| SFab HC | 7 | 7 | 7 | 7 | 126 | 126 | 131 |
| HINGE(3) | | | | | | | |
| CH2(3) | 8 | 8 | 8 | 8 | 8 | 8 | 8 |
| CH3 (3) | 106 | 111 | 111 | 111 | 127 | 127 | 127 |
| SFab | 146 | 112 | 181 | 118 | 185 | 185 | 186 |
| HC(3) | | | | | | | |
| VL(3) | IL4-0002/VL | IL13- | P40- | IL13- | P40- | P40- | P40- |
| | | 0001/VL | 0003/VL | 0271/VL | 0003/VL | 0003/VL | 0003/VL |
| V_{L}(3) | 20 | 54 | 175 | 59 | 175 | 175 | 175 |
| CL(3) | 108 | 113 | 16 | 113 | 16 | 16 | 16 |
| SFab | 109 | 196 | 182 | 120 | 176 | 176 | 176 |
| LC(3) | | | | | | | |

TABLE 87
SEQUENCE LIST

| SEQ | | |
|---|---|---|
| ID | | |
| NO: | Description | Sequence |
| 1 | IL4-1284 CDRH1 | GFSLSTSGMGVS |
| | [IL13433-1284 CDRH1] | |
| | (hu3B9 CDRH1) | |
| 2 | IL4-1284 CDRH2 | HIYWDDDKRYNPSLKS |
| | [IL13433-1284 CDRH2] | |
| | (hu3B9 CDRH2) | |
| 3 | IL4-1284 CDRH3 | RETVFYWYFDV |
| | [IL13433-1284 CDRH3] | |
| | (hu3B9 CDRH3) | |
| 4 | IL4-1284 JH [IL13433- | WGRGTPVTVSS |
| | 1284 JH] (hu3B9 JH) | |
| 5 | IL4-1284 V_{H} | QVTLRESGPALVKPTQTLTLTCTFSGFSLSTSGMGVSWIRQPPGKGLEWLAHIYWD |
| | [IL13433-1284 V_{H}] | DDKRYNPSLKSRLTISKDTSRNQVVLTMTNMDPVDTATYYCARRETVFYWYFDVWG |
| | (hu3B9 V_{H}) | RGTPVTVSS |
| 6 | CH1 | ASTKGPSVFPLAPSSKSTSGGTAALGCLVKDYFPEPVTVSWNSGALTSGVHTFPAV |
| | | LQSSGLYSLSSVVTVPSSSLGTQTYICNVNHKPSNTKVDKKV |
| 7 | HINGE | EPKSCDKTHTCPPCP |
| 8 | CH2 | APEAAGAPSVFLFPPKPKDTLMISRTPEVTCVVVDVSHEDPEVKENWYVDGVEVHN |
| | | AKTKPREEQYNSTYRVVSVLTVLHQDWLNGKEYKCKVSNKALPAPIEKTISKAK |
| 9 | CH3 | GQPREPQVYTLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSLSLSPGK |
| 10 | IL4-1284 HC | QVTLRESGPALVKPTQTLTLTCTFSGFSLSTSGMGVSWIRQPPGKGLEWLAHIYWD |
| | [IL13433-1284 HC] | DDKRYNPSLKSRLTISKDTSRNQVVLTMTNMDPVDTATYYCARRETVFYWYFDVWG |
| | (hu3B9 HC) | RGTPVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| | | DKTHTCPPCPAPEAAGAPSVELFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PGK |
| 11 | IL4-1284 CDRL1 | KASQSVDYDGDSYMN |
| | [IL13433-1284 CDRL1] | |
| | (hu3B9 CDRL1) | |
| 12 | IL4-1284 CDRL2 | AASNLES |
| | [IL13433-1284 CDRL2] | |
| | (hu3B9 CDRL2) | |
| 13 | IL4-1284 CDRL3 | QQSNEDPPR |
| | [IL13433-1284 CDRL3] | |
| | (hu3B9 CDRL3) | |
| 14 | IL4-1284 JK | FGGGTKVEIK |
| | [IL13433-1284 JK] | |
| | (hu3B9 JK) | |
| 15 | IL4-1284 V_{L} | DIVMTQSPDSLAVSLGERATINCKASQSVDYDGDSYMNWYQQKPGQPPKLLIYAAS |
| | [IL13433-1284 V_{L}] | NLESGVPDRFSGSGSGTDFTLTISSLQAEDVAVYYCQQSNEDPPRFGGGTKVEIK |
| | (hu3B9 V_{L}) | |
| 16 | CL (Kappa) | (R)TVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSENRGEC |
| 17 | IL4-1284 LC | DIVMTQSPDSLAVSLGERATINCKASQSVDYDGDSYMNWYQQKPGQPPKLLIYAAS |
| | [IL13433-1284 LC] | NLESGVPDRFSGSGSGTDFTLTISSLQAEDVAVYYCQQSNEDPPRFGGGTKVEIKR |
| | (hu3B9 LC) | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGEC |
| 18 | IL4-1305 CDRH1 | GFSLSNFGEGLS |
| | [IL13433-1305 CDRH1] | |
| | (hu3B9_G07_VLv2.9 | |
| | CDRH1) | |
| 19 | IL4-1305 V_{H} | EVTLRESGPALVKPTQTLTLTCTFSGFSLSNFGEGLSWIRQPPGKGLEWLAHIYWD |
| | [IL13433-1305 V_{H}] | DDKRYNPSLKSRLTISKDTSRNQVVLTMTNMDPVDTATYYCARRETVFYWYFDVWG |
| | (hu3B9_G07-Q1E_VLv2.9 | RGTPVTVSS |
| | V_{H}) | |
| 20 | IL4-1305 V_{L} | DIQMTQSPSSLSASVGDRVTITCKASQSVDEDGDSYMNWYQQKPGKAPKLLIYAAS |
| | [IL13433-1305 V_{L}] | NLESGVPSRFSGSGSGTDFTLTISSLQPEDFATYYCQQSNEDPPTFGGGTKVEIK |
| | (hu3B9_G07_VLv2.9 V_{L}) | |
| 21 | IL4-0002 JH | WGQGTTVTVSS |
| | [IL13433-0002 JH] | |
| | (anti-IL-4 JH) | |
| 22 | IL4-0002 V_{H} | EVTLRESGPALVKPTQTLTLTCTFSGFSLSNFGEGLSWIRQPPGKGLEWL |
| | [IL13433-0002 V_{H}] | AHIYWDDDKRYNPSLKSRLTISKDTSRNQVVLTMTNMDPVDTATYYCARR |
| | (anti-IL-4 V_{H}) | ETVFYWYFDVWGQGTTVTVSS |
| 23 | IL4-0002 HC | EVTLRESGPALVKPTQTLTLTCTFSGFSLSNFGEGLSWIRQPPGKGLEWLAHIYWD |
| | [IL13433-0002 HC] | DDKRYNPSLKSRLTISKDTSRNQVVLTMTNMDPVDTATYYCARRETVFYWYFDVWG |
| | (anti-IL-4 HC) | QGTTVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| | | DKTHTCPPCPAPEAAGAPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PGK |
| 24 | IL4-1040 CDRL1 | RASQSVDEEGDSYMN |
| | [IL13433-1040 CDRL1] | |
| | (anti-IL-4 CDRL1) | |
| 25 | IL4-1040 CDRL3 | QQSNKDPPT |
| | [IL13433-1040 CDRL3] | |
| | (anti-IL-4 CDRL3) | |
| 26 | IL4-1040 V_{L} | DIQMTQSPSSLSASVGDRVTITCRASQSVDEEGDSYMNWYQQKPGKAPKLLIYAAS |
| | [IL13433-1040 V_{L}] | NLESGVPSRFSGSGSGTDFTLTISSLQPEDFATYYCQQSNKDPPTFGGGTKVEIK |
| | (anti-IL-4 V_{L}) | |
| 27 | IL4-1040 LC | DIQMTQSPSSLSASVGDRVTITCRASQSVDEEGDSYMNWYQQKPGKAPKL |
| | [IL13433-1040 LC] | LIYAASNLESGVPSRFSGSGSGTDFTLTISSLQPEDFATYYCQQSNKDPP |
| | (anti-IL-4 LC) | TFGGGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACEV |
| | | THQGLSSPVTKSENRGEC |
| 28 | IL4-0157 V_{H} | EVTLRESGPALVKPTQTLTLTCTFSGFSLSNFGEGLSWIRQPPGKGLEWLAHIYWD |
| | [IL13433-0157 V_{H}] | DDKRYSTSLKTRLTISKDTSRNQVVLTMTNMDPVDTATYYCARRETVFYWYFDVWG |
| | | QGTTVTVSS |
| 29 | IL4-0157 V_{L} | DIQMTQSPSSLSASVGDRVTITCRASQSVDEEGDSYMNWYQQKPGKAPKLLIYAAS |
| | [IL13433-0157 V_{L}] | NLESGVPSRFSGSGSGTDFTLTISSLQPEDFATYYCQQSHKDPPTFGGGTKVEIK |
| 30 | IL4-0749 V_{L} | DIQMTQSPSSLSASVGDRVTITCRASQSVDEEGDSYMNWYQQKPGKAPKLLIYAAS |
| | [IL13433-0749 V_{L}] | NLESGVPSRFSGSGSGTDFTLTISSLQPEDFATYYCQQSNEDPPTFGGGTKVEIK |
| 31 | IL13-1283 CDRH1 | GFTFISYAMS |
| | [IL13433-1283 CDRH1} | |
| | or (IMA-638 CDRH1) | |
| 32 | IL13-1283 CDRH2 | SISSGGNTYYPDSVKG |
| | [IL13433-1283 CDRH2] | |
| | or 9IMA-638 CDRH2) | |
| 33 | IL13-1283 CDRH3 | LDGYYFGFAY |
| | [IL13433-1283 CDRH3] | |
| | or (IMA-638 CDRH3) | |
| 34 | IL13-1283 V_{H} | EVQLVESGGGLVQPGGSLRLSCAASGFTFISYAMSWVRQAPGKGLEWVASISSGGN |
| | [IL13433-1283 V_{H}] or | TYYPDSVKGRFTISRDNAKNSLYLQMNSLRAEDTAVYYCARLDGYYFGFAYWGQGT |
| | (IMA-638 V_{H}) | LVTVSS |
| 35 | IL13-1283 HC | EVQLVESGGGLVQPGGSLRLSCAASGFTFISYAMSWVRQAPGKGLEWVASISSGGN |
| | [IL13433-1283 HC] or | TYYPDSVKGRFTISRDNAKNSLYLQMNSLRAEDTAVYYCARLDGYYFGFAYWGQGT |
| | (IMA-638 HC) | LVTVSSASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPKSCDKT |
| | | HTCPPCPAPEAAGAPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNKALPAPIEKT |
| | | ISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| 36 | IL13-1283 CDRL1 | KASESVDNYGKSLMH |
| | [IL13433-1283 CDRL1] | |
| | or (IMA-638 CDRL1) | |
| 37 | IL13-1283 CDRL2 | RASNLES |
| | [IL13433-1283 CDRL2] | |
| | or (IMA-638 CDRL2) | |
| | Also [IL13433-1306 | |
| | CDRL2] (mu13.4 CDRL2) | |
| 38 | IL13-1283 CDRL3 | QQSNEDPWT |
| | [IL13433-1283 CDRL3] | |
| | or (IMA-638 CDRL3) | |
| | Also [IL13433-1306 | |
| | CDRL3] (mu13.4 CDRL3) | |
| 39 | IL13-1283 V_{L} | DIQMTQSPSSLSASVGDRVTITCKASESVDNYGKSLMHWYQQKPGKAPKLLIYRAS |
| | [IL13433-1283 V_{L}} or | NLESGVPSRFSGSGSGTDFTLTISSLQPEDFATYYCQQSNEDPWTFGGGTKVEIK |
| | (IMA-638 V_{L}) | |
| 40 | IL13-1283 LC | DIQMTQSPSSLSASVGDRVTITCKASESVDNYGKSLMHWYQQKPGKAPKLLIYRAS |
| | [IL13433-1283] LC or | NLESGVPSRFSGSGSGTDFTLTISSLQPEDFATYYCQQSNEDPWTFGGGTKVEIKR |
| | (IMA-638 LC) | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGEC |
| 41 | IL13-1306 CDRH1 | GFTFSSYAMS |
| | [IL13433-1306 CDRH1] | |
| | (mu13.4 CDRH1) | |
| 42 | IL13-1306 CDRH2 | SISSGDTTYYPDSVKG |
| | [IL13433-1306 CDRH2] | |
| | (mu13.4 CDRH2) | |
| 43 | IL13-1306 CDRH3 | LDGYYFGFPY |
| | [IL13433-1306 CDRH3] | |
| | (mu13.4 CDRH3) | |
| 44 | IL13-1306 V_{H} | EVKLVESGGDLVKPGGSLKLSCAASGFTFSSYAMSWVRQSPDKRLEWVASISSGDT |
| | [IL13433-1306 V_{H}] | TYYPDSVKGRFTISRDNARNILYLQMTSLRSEDTAMYYCARLDGYYFGFPYWGQGT |
| | (mu13.4 V_{H}) | LVTVSA |
| 45 | IL13-1306 CDRL1 | KASESVDHSGSSLMH |
| | [IL13433-1306 CDRL1] | |
| | (mu13.4 CDRL1) | |
| 46 | IL13-1306 V_{L} | DTVLTQSPASLAVSLGQRATISCKASESVDHSGSSLMHWYQQKPGQSPKLLIYRAS |
| | [IL13433-1306 V_{L}] | NLESGIPARFSGSGSRTDFTLTINPVEADDVATYYCQQSNEDPWTFGGGTKLEIK |
| | (mu13.4 V_{L}) | |
| 47 | IL13-1307 JH | WGQGTLVTVSS |
| | [IL13433-1307 JH] | |
| | (hu13.4 JH) | |
| 48 | IL13-1307 V_{H} | EVQLVESGGGLVQPGGSLRLSCAASGFTFSSYAMSWVRQAPGKGLEWVASISSGDT |
| | [IL13433-1307 V_{H}] | TYYPDSVKGRFTISRDNAKNSLYLQMNSLRAEDTAVYYCARLDGYYFGFPYWGQGT |
| | (hu13.4 V_{H}) | LVTVSS |
| 49 | IL13-1307 V_{L} | DIQMTQSPSSLSASVGDRVTITCKASESVDHSGSSLMHWYQQKPGKAPKLLIYRAS |
| | [IL13433-1307 V_{L}] | NLESGVPSRFSGSGSGTDFTLTISSLQPEDFATYYCQQSNEDPWTFGGGTKVEIK |
| | (hu13.4 V_{L}) | |
| 50 | IL13-0001 CDRH3 | NEGYYFGLTL |
| | [IL1343-0001 CDRH3] | |
| 51 | IL13-0001 V_{H} | EVQQLVESGGGLVQPGGSLRLSCAASGFTFSSYAMSWVRQAPGKGLEWVASISSGDT |
| | [IL1343-0001 V_{H}] | TYYPDSVKGRFTISRDNAKNSLYLQMNSLRAEDTAVYYCARNEGYYFGLTLWGQGT |
| | | LVTVSS |
| 52 | IL13-0001 HC | EVQLVESGGGLVQPGGSLRLSCAASGFTFSSYAMSWVRQAPGKGLEWVASISSGDT |
| | [IL1343-0001 HC] | TYYPDSVKGRFTISRDNAKNSLYLQMNSLRAEDTAVYYCARNEGYYFGLTLWGQGT |
| | | LVTVSSASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPKSCDKT |
| | | HTCPPCPAPEAAGAPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNKALPAPIEKT |
| | | ISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| 53 | IL13-0001 CDRL1 | KASESVDHFGWSLVH |
| | [IL1343-0001 CDRL1] | |
| 54 | IL13-0001 V_{L} | DIQMTQSPSSLSASVGDRVTITCKASESVDHFGWSLVHWYQQKPGKAPKLLIYRAS |
| | [IL1343-0001 V_{L}] | NLESGVPSRFSGSGSGTDFTLTISSLQPEDFATYYCQQSNEDPWTFGGGTKVEIK |
| 55 | IL13-0001 LC | DIQMTQSPSSLSASVGDRVTITCKASESVDHFGWSLVHWYQQKPGKAPKLLIYRAS |
| | [IL1343-0001 LC] | NLESGVPSRFSGSGSGTDFTLTISSLQPEDFATYYCQQSNEDPWTFGGGTKVEIKR |
| | | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGEC |
| 56 | IL13-0259 JH | WGRGTLVTVSS |
| | [IL13433-0259 JH] | |
| 57 | IL13-0259 V_{H} | EVQLVESGGGLVQPGGSLRLSCAASGFTFSSYAMSWVRQAPGKGLEWVASISTGDT |
| | [IL13433-0259 V_{H}] | TYYPDSVKGRFTISRDNAKNSLYLQMNSLRAEDTAVYYCARNEGYYFGLTQWGRGT |
| | | LVTVSS |
| 58 | IL13-0259 V_{L} | DIQMTQSPSSLSASVGDRVTITCRASQSVDHFGWSLVHWYQQKPGKAPKLLIYRAS |
| | [IL13433-0259 V_{L}] | NLESGVPSRFSGSGSGTDFTLTISSLQPEDFATYYCQQSNEDPWTFGGGTKVEIK |
| 59 | IL13-0270 V_{L} | DIQMTQSPSSLSASVGDRVTITCRASESVDHFGWSLVHWYQQKPGKAPKLLIYRAD |
| | [IL13433-0270 V_{L}] | SLETGVPSRFSGSGSGTDFTLTISSLQPEDFATYYCQQSNEDPWTFGGGTKVEIK |
| 60 | IL33-0232 CDRH1 | GFTFSSYWMY |
| | (IL33-158-152 CDRH1) | |
| 61 | IL33-0232 CDRH2 | AITPNAGEDYYPESVKG |
| | (IL33-158-152 CDRH2) | |
| 62 | IL33-0232 CDRH3 | GHYYYTSYSLGY |
| | (IL33-158-152 CDRH3) | |
| 63 | IL33-0232 V_{H} | EVQLVESGGGLVQPGGSLRLSCAASGFTFSSYWMYWVRQAPGKGLEWVAAITPNAG |
| | (IL33-158-152 V_{H}) | EDYYPESVKGRFTISRDNAKNSLYLQMNSLRAEDTAVYYCARGHYYYTSYSLGYWG |
| | | QGTLVTVSS |
| 64 | IL33-0232 HC | EVQLVESGGGLVQPGGSLRLSCAASGFTFSSYWMYWVRQAPGKGLEWVAAITPNAG |
| | (IL33-158-152 HC) | EDYYPESVKGRFTISRDNAKNSLYLQMNSLRAEDTAVYYCARGHYYYTSYSLGYWG |
| | | QGTLVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| | | DKTHTCPPCPAPEAAGAPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PGK |
| 65 | IL33-0232 CDRL1 | KASQNINKHLD |
| | (IL33-158-152 CDRL1) | |
| 66 | IL33-0232 CDRL2 | FTNNLQT |
| | (IL33-158-152 CDRL2) | |
| 67 | IL33-0232 CDRL3 | FQYNQGWT |
| | (IL33-158-152 CDRL3) | |
| 68 | IL33-0232 V_{L} | DIQMTQSPSSLSASVGDRVTITCKASQNINKHLDWYQQKPGKAPKLLIYFTNNLQT |
| | (IL33-158-152 V_{L}) | GVPSRFSGSGSGTDFTLTISSLQPEDFATYYCFQYNQGWTFGGGTKVEIK |
| 69 | IL33-0232 LC | DIQMTQSPSSLSASVGDRVTITCKASQNINKHLDWYQQKPGKAPKLLIYFTNNLQT |
| | (IL33-158-152 LC) | GVPSRFSGSGSGTDFTLTISSLQPEDFATYYCFQYNQGWTFGGGTKVEIKRTVAAP |
| | | SVFIFPPSDEQLKSGTASVVCLLNNFYPREAKVQWKVDNALQSGNSQESVTEQDSK |
| | | DSTYSLSSTLTLSKADYEKHKVYACEVTHQGLSSPVTKSENRGEC |
| 70 | IL33-0224 CDRL1 | RASQPISKHLD |
| | [IL13433-0224 CDRL1] | |
| 71 | IL33-0224 VL | DIQMTQSPSSLSASVGDRVTITCRASQPISKHLDWYQQKPGKAPKLLIYFTNNLQT |
| | [IL13433-0224 VL] | GVPSRFSGSGSGTDFTLTISSLQPEDFATYYCFQYNQGWTFGGGTKVEIK |
| 72 | IL33-0726 CDRH3 | GQYYYTKYSLGY |
| | [IL13433-0726 CDRH3] | |
| 73 | IL33-0726 V_{H} | EVQLVESGGGLVQPGGSLRLSCAASGFTFSSYWMYWVRQAPGKGLEWVAAITPNAG |
| | [IL13433-0726 V_{H}] | EDYYPESVKGRFTISRDNAKNSLYLQMNSLRAEDTAVYYCARGQYYYTKYSLGYWG |
| | | QGTLVTVSS |
| 74 | IL33-0726 HC | EVQLVESGGGLVQPGGSLRLSCAASGFTFSSYWMYWVRQAPGKGLEWVAAITPNAG |
| | [IL13433-0726 HC] | EDYYPESVKGRFTISRDNAKNSLYLQMNSLRAEDTAVYYCARGQYYYTKYSLGYWG |
| | | QGTLVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| | | DKTHTCPPCPAPEAAGAPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PGK |
| 75 | IL33-0726 CDRL1 | RASQPIHNHLD |
| | [IL13433-0726 CDRL1] | |
| 76 | IL33-0726 CDRL2 | FGKNLQE |
| | [IL13433-0726 CDRL2] | |
| 77 | IL33-0726 CDRL3 | FQYKKGWS |
| | [IL13433-0726 CDRL3] | |
| 78 | IL33-0726 V_{L} | DIQMTQSPSSLSASVGDRVTITCRASQPIHNHLDWYQQKPGKAPKLLIYFGKNLQE |
| | [IL13433-0726 V_{L}] | GVPSRFSGSGSGTDFTLTISSLQPEDFATYYCFQYKKGWSFGGGTKVEIK |
| 79 | IL33-0726 LC | DIQMTQSPSSLSASVGDRVTITCRASQPIHNHLDWYQQKPGKAPKLLIYFGKNLQE |
| | [IL13433-0726 LC] | GVPSRFSGSGSGTDFTLTISSLQPEDFATYYCFQYKKGWSFGGGTKVEIKRTVAAP |
| | | SVFIFPPSDEQLKSGTASVVCLLNNFYPREAKVQWKVDNALQSGNSQESVTEQDSK |
| | | DSTYSLSSTLTLSKADYEKHKVYACEVTHQGLSSPVTKSENRGEC |
| 80 | IL33-0721 V_{H} | EVQLVESGGGLVQPGGSLRLSCAASGFEFSHYWMYWVRQAPGKGLEWVAAITPNAG |
| | | EDYYPESVKGRFTISRDNAKNSLYLQMNSLRAEDTAVYYCARGYYRYTKYSLGYWG |
| | | QGTLVTVSS |
| 81 | IL33-0721 V_{L} | DIQMTQSPSSLSASVGDRVTITCRASQPIHQFLDWYQQKPGKAPKLLIYFGHILQE |
| | | GVPSRFSGSGSGTDFTLTISSLQPEDFATYYCFQYKQGWSFGGGTKVEIK |
| 82 | TSLP-0001 CDRH1 | GFTFRTYGMH |
| | TSLP-0100 CDRH1 | |
| | TSLP-0156 CDRH1 | |
| | TSLP-0260 CDRH1 | |
| | TSLP-0821 CDRH1 | |
| | TSLP-0855 CDRH1 | |
| | TSLP-0871 CDRH1 | |
| | TSLP-0875 CDRH1 | |
| | TSLP-0820 CDRH1 | |
| | TSLP-2000 CDRH1 | |
| | TSLP-2002 CDRH1 | |
| | TSLP-2004 CDRH1 | |
| | IL413TSLP-1024 CDRH1 | |
| | IL413TSLP-1028 CDRH1 | |
| | IL413TSLP-1037 CDRH1 | |
| 83 | TSLP-0001 CDRH2 | VIWYDGSNKHYADSVKG |
| | TSLP-0100 CDRH2 | |
| | TSLP-0156 CDRH2 | |
| | TSLP-0260 CDRH2 | |
| | TSLP-0821 CDRH2 | |
| | TSLP-0855 CDRH2 | |
| | TSLP-0871 CDRH2 | |
| | TSLP-0875 CDRH2 | |
| | TSLP-0820 CDRH1 | |
| | TSLP-2000 CDRH1 | |
| | TSLP-2002 CDRH1 | |
| | TSLP-2004 CDRH1 | |
| | IL413TSLP-1024 CDRH2 | |
| | IL413TSLP-1028 CDRH2 | |
| | IL413TSLP-1037 CDRH2 | |
| 84 | TSLP-0001 CDRH3 | APQWELVHEAFDI |
| | TSLP-0100 CDRH3 | |
| 85 | TSLP-0156 CDRH3 | APQWYLVHEAFDI |
| | TSLP-0260 CDRH3 | |
| | TSLP-0821 CDRH3 | |
| | TSLP-0855 CDRH3 | |
| | TSLP-0871 CDRH3 | |
| | TSLP-0875 CDRH3 | |
| | TSLP-0820 CDRH1 | |
| | TSLP-2000 CDRH1 | |
| | TSLP-2002 CDRH1 | |
| | TSLP-2004 CDRH1 | |
| | IL413TSLP-1024 CDRH3 | |
| | IL413TSLP-1028 CDRH3 | |
| | IL413TSLP-1037 CDRH3 | |
| 86 | TSLP-0001 CDRL1 | GGNNLGSKSVH |
| | TSLP-0100 CDRL1 | |
| | TSLP-0156 CDRL1 | |
| | TSLP-0260 CDRL1 | |
| | TSLP-0821 CDRL1 | |
| | TSLP-0855 CDRL1 | |
| | TSLP-0871 CDRL1 | |
| | TSLP-0875 CDRL1 | |
| | TSLP-0820 CDRL1 | |
| | TSLP-2000 CDRL1 | |
| | TSLP-2002 CDRL1 | |
| | TSLP-2004 | |
| | CDRL1IL413TSLP-1024 | |
| | CDRL1 | |
| | IL413TSLP-1028 CDRL1 | |
| | IL413TSLP-1037 CDRL1 | |
| 87 | TSLP-0001 CDRL2 | DDSDRPS |
| | TSLP-0100 CDRL2 | |
| | TSLP-0156 CDRL2 | |
| | TSLP-0260 CDRL2 | |
| | TSLP-0821 CDRL2 | |
| | TSLP-0855 CDRL2 | |
| | TSLP-0820 CDRL2 | |
| | TSLP-2000 CDRL2 | |
| | TSLP-2004 CDRL2 | |
| 88 | TSLP-0871 CDRL2 | DDKDRPS |
| | TSLP-0875 CDRL2 | |
| | TSLP-2002 CDRL2 | |
| | IL413TSLP-1024 CDRL2 | |
| 89 | TSLP-0001 CDRL3 | QVWDSSSDHVV |
| | TSLP-0100 CDRL3 | |
| | TSLP-0156 CDRL3 | |
| | TSLP-0821 CDRL3 | |
| | TSLP-0820 CDRL3 | |
| 90 | TSLP-0875 CDRL3 | QVWDSKSDHVV |
| | TSLP-2002 CDRL3 | |
| | IL413TSLP-1024 CDRL3 | |
| 91 | TSLP-0001 VH | QMQLVESGGGVVQPGRSLRLSCAASGFTFRTYGMHWVRQAPGKGLEWVAV |
| | | IWYDGSNKHYADSVKGRFTITRDNSKNTLNLQMNSLRAEDTAVYYCARAP |
| | | QWELVHEAFDIWGQGTMVTVSS |
| 92 | TSLP-0156 VH | QMQLVESGGGVVQPGRSLRLSCAASGFTFRTYGMHWVRQAPGKGLEWVAVIWYDGS |
| | TSLP-0260 VH | NKHYADSVKGRFTISRDNSKNTLYLQMNSLRAEDTAVYYCARAPQWYLVHEAFDIW |
| | TSLP-0855 VH | GQGTMVTVSS |
| | TSLP-0871 VH | |
| | TSLP-0875 VH | |
| | IL413TSLP-1024 VH | |
| 93 | TSLP-0001 VL | SYVLTQPPSVSVAPGQTARITCGGNNLGSKSVHWYQQKPGQAPVLVVYDDSDRPSW |
| | TSLP-0100 VL | IPERFSGSNSGNTATLTISRGEAGDEADYYCQVWDSSSDHVVFGGGTKLTVL |
| | TSLP-0156 VL | |
| | TSLP-0820 VL | |
| | TSLP-0821 VL | |
| 94 | TSLP-0875 VL | SYVLTQPPSVSVAPGQTARITCGGNNLGSKSVHWYQQKPGQAPVLVVYDDKDRPSW |
| | IL413TSLP-1024 VL | IPERFSGSNSGNTATLTISRGEAGDEADYYCQVWDSKSDHVVFGGGTKLTVL |
| 95 | Human lambda chain | GQPKAAPSVTLFPPSSEELQANKATLVCLISDFYPGAVTVAWKADSSPVKAGVETT |
| | constant region | TPSKQSNNKYAASSYLSLTPEQWKSHRSYSCQVTHEGSTVEKTVAPTECS |
| 96 | TSLP-0001 HC | QMQLVESGGGVVQPGRSLRLSCAASGFTFRTYGMHWVRQAPGKGLEWVAVIWYDGS |
| | | NKHYADSVKGRFTITRDNSKNTLNLQMNSLRAEDTAVYYCARAPQWELVHEAFDIW |
| | | GQGTMVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKKVEPKS |
| | | CDKTHTCPPCPAPEAAGAPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSLSL |
| | | SPGK |
| 97 | TSLP-0156 HC | QMQLVESGGGVVQPGRSLRLSCAASGFTFRTYGMHWVRQAPGKGLEWVAVIWYDGS |
| | TSLP-0260 HC | NKHYADSVKGRFTISRDNSKNTLYLQMNSLRAEDTAVYYCARAPQWYLVHEAFDIW |
| | TSLP-0855 HC | GQGTMVTVSSASTKGPSVFPLAPSSKSTSGGTAALGCLVKDYFPEPVTVSWNSGAL |
| | TSLP-0871 HC | TSGVHTFPAVLQSSGLYSLSSVVTVPSSSLGTQTYICNVNHKPSNTKVDKKVEPKS |
| | TSLP-0875 HC | CDKTHTCPPCPAPEAAGAPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSLSL |
| | | SPGK |
| 98 | TSLP-0001 LC | SYVLTQPPSVSVAPGQTARITCGGNNLGSKSVHWYQQKPGQAPVLVVYDDSDRPSW |
| | TSLP-0100 LC | IPERFSGSNSGNTATLTISRGEAGDEADYYCQVWDSSSDHVVFGGGTKLTVLGQPK |
| | TSLP-0156 LC | AAPSVTLFPPSSEELQANKATLVCLISDFYPGAVTVAWKADSSPVKAGVETTTPSK |
| | TSLP-0821 LC | QSNNKYAASSYLSLTPEQWKSHRSYSCQVTHEGSTVEKTVAPTECS |
| | IL413TSLP-0003 | |
| | IL413TSLP-0004 | |
| 99 | TSLP-0875 LC | SYVLTQPPSVSVAPGQTARITCGGNNLGSKSVHWYQQKPGQAPVLVVYDDKDRPSW |
| | | IPERFSGSNSGNTATLTISRGEAGDEADYYCQVWDSKSDHVVFGGGTKLTVLGQPK |
| | | AAPSVTLFPPSSEELQANKATLVCLISDFYPGAVTVAWKADSSPVKAGVETTTPSK |
| | | QSNNKYAASSYLSLTPEQWKSHRSYSCQVTHEGSTVEKTVAPTECS |
| 100 | JH (TSLP) | WGQGTMVTVSS |
| 101 | Jλ (TSLP) | FGGGTKLTVL |
| 102 | IL13433-0006 Upper | EPKSC |
| | HINGE (1) | |
| 103 | IL13433-0006 mFd(1) | EVQLVESGGGLVQPGGSLRLSCAASGFTFSSYWMYWVRQAPGKGLEWVAAITPNAG |
| | | EDYYPESVKGRFTISRDNAKNSLYLQMNSLRAEDTAVYYCARGHYYYTSYSLGYWG |
| | | QGTLVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| 104 | IL13433-0006 LINKER | GGGGS |
| 105 | IL13433-0006 CH1 (2) | ASTKGPSVFPEAPSSKSTSGGTAALGCLVKDYFPEPVTVSWNSGALTSGVHTFPAV |
| | | LQSSGLYSLGSVVTVPSSSLGTQTYICNVNHKPSNTKVDKKV |
| 106 | IL13433-0006 CH3 (2) | GQPREPQVYTLPPCREEMTKNQVSLSCAVKGFYPSDIAVEWESNGQPENNYKTTPP |
| | | VLDSDGSFFLVSKLTVDKSRWQQGNVFSCSVMHEALHNHYTQKSLSLSPGK |
| 107 | IL13433-0006 DFab | DIQMTQSPSSLSASVGDRVTITCKASQNINKHLDWYQQKPGKAPKLLIYFTNNLQT |
| | LC (1)-HC (2) | GVPSRFSGSGSGTDFTLTISSLQPEDFATYYCFQYNQGWTFGGGTKVEIKRTVAAP |
| | | SVFIFPPSDEQLKSGTASVVCLLNNFYPREAKVQWKVDNALQSGNSQESVTEQDSK |
| | | DSTYSLSSTLTLSKADYEKHKVYACEVTHQGLSSPVTKSENRGECGGGGSEVTLRE |
| | | SGPALVKPTQTLTLTCTFSGFSLSNFGEGLSWIRQPPGKGLEWLAHIYWDDDKRYN |
| | | PSLKSRLTISKDTSRNQVVLTMTNMDPVDTATYYCARRETVFYWYFDVWGQGTTVT |
| | | VSSASTKGPSVFPEAPSSKSTSGGTAALGCLVKDYFPEPVTVSWNSGALTSGVHTF |
| | | PAVLQSSGLYSLGSVVTVPSSSLGTQTYICNVNHKPSNTKVDKKVEPKSCDKTHTC |
| | | PPCPAPEAAGAPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPAPIEKTISK |
| | | AKGQPREPQVYTLPPCREEMTKNQVSLSCAVKGFYPSDIAVEWESNGQPENNYKTT |
| | | PPVLDSDGSFFLVSKLTVDKSRWQQGNVFSCSVMHEALHNHYTQKSLSLSPGK |
| 108 | IL13433-0006 CL (2) | (R)TVAAPSVFIFPPSDEQLKSGTASVSCLLNNFYPREAKVQWKVDNALQSGNSQE |
| | | SVTEQDSKDSTYSLKSTLTLSKADYEKHKVYACEVTHQGLSSPVTKSENRGEC |
| 109 | IL13433-0006 LC (2) | DIQMTQSPSSLSASVGDRVTITCKASQSVDEDGDSYMNWYQQKPGKAPKLLIYAAS |
| | | NLESGVPSRFSGSGSGTDFTLTISSLQPEDFATYYCQQSNEDPPTFGGGTKVEIKR |
| | | TVAAPSVFIFPPSDEQLKSGTASVSCLLNNFYPREAKVQWKVDNALQSGNSQESVT |
| | | EQDSKDSTYSLKSTLTLSKADYEKHKVYACEVTHQGLSSPVTKSENRGEC |
| 110 | IL13433-0006 CH1 (3) | ASTKGPSVFPKAPSSKSTSGGTAALGCLVKDYFPEPVTVSWNSGALTSGVHTFPAV |
| | | LQSSGLYSLSSSVTVPSSSLGTQTYICNVNHKPSNTKVDKKV |
| 111 | IL13433-0006 CH3 (3) | GQPREPQVCTLPPSREEMTKNQVSLWCLVKGFYPSDIAVEWESNGQPENNYKTTPP |
| | | VLDSDGSFFLYSKLTVDKSRWQQGNVFSCSVMHEALHNHYTQKSLSLSPGK |
| 112 | IL13433-0006 HC (3) | EVQLVESGGGLVQPGGSLRLSCAASGFTFSSYAMSWVRQAPGKGLEWVASISSGDT |
| | | TYYPDSVKGRFTISRDNAKNSLYLQMNSLRAEDTAVYYCARNEGYYFGLTLWGQGT |
| | | LVTVSSASTKGPSVFPKAPSSKSTSGGTAALGCLVKDYFPEPVTVSWNSGALTSGV |
| | | HTFPAVLQSSGLYSLSSSVTVPSSSLGTQTYICNVNHKPSNTKVDKKVEPKSCDKT |
| | | HTCPPCPAPEAAGAPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNKALPAPIEKT |
| | | ISKAKGQPREPQVCTLPPSREEMTKNQVSLWCLVKGFYPSDIAVEWESNGQPENNY |
| | | KTTPPVLDSDGSFFLYSKLTVDKSRWQQGNVFSCSVMHEALHNHYTQKSLSLSPGK |
| 113 | IL13433-0006 CL (3) | (R)TVAAPSVFIFPPSDEQLKSGTASVSCLLNNFYPREAKVQWKVDNALQSGNSQE |
| | | SVTEQDSKDSTYSLDSTLTLSKADYEKHKVYACEVTHQGLSSPVTKSENRGEC |
| 114 | IL13433-0606 CH3 (2) | GQPREPQVYTLPPCREEMTKNQVSLSCAVKGFYPSDIAVEWESNGQPENNYKTTPP |
| | | VLDSDGSFFLVSKLTVDKSRWQQGNVFSCSVLHEALHSHYTQKSLSLSPGK |
| 115 | IL13433-0606 DFab | DIQMTQSPSSLSASVGDRVTITCRASQPISKHLDWYQQKPGKAPKLLIYFTNNLQT |
| | LC (1)-HC (2) | GVPSRFSGSGSGTDFTLTISSLQPEDFATYYCFQYNQGWTFGGGTKVEIKRTVAAP |
| | | SVFIFPPSDEQLKSGTASVVCLLNNFYPREAKVQWKVDNALQSGNSQESVTEQDSK |
| | | DSTYSLSSTLTLSKADYEKHKVYACEVTHQGLSSPVTKSENRGECGGGGSEVTLRE |
| | | SGPALVKPTQTLTLTCTFSGFSLSNFGEGLSWIRQPPGKGLEWLAHIYWDDDKRYS |
| | | TSLKTRLTISKDTSRNQVVLTMTNMDPVDTATYYCARRETVFYWYFDVWGQGTTVT |
| | | VSSASTKGPSVFPEAPSSKSTSGGTAALGCLVKDYFPEPVTVSWNSGALTSGVHTF |
| | | PAVLQSSGLYSLGSVVTVPSSSLGTQTYICNVNHKPSNTKVDKKVEPKSCDKTHTC |
| | | PPCPAPEAAGAPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPAPIEKTISK |
| | | AKGQPREPQVYTLPPCREEMTKNQVSLSCAVKGFYPSDIAVEWESNGQPENNYKTT |
| | | PPVLDSDGSFFLVSKLTVDKSRWQQGNVFSCSVLHEALHSHYTQKSLSLSPGK |
| 116 | IL13433-0606 LC (2) | DIQMTQSPSSLSASVGDRVTITCRASQSVDEEGDSYMNWYQQKPGKAPKLLIYAAS |
| | | NLESGVPSRFSGSGSGTDFTLTISSLQPEDFATYYCQQSHKDPPTFGGGTKVEIKR |
| | | TVAAPSVFIFPPSDEQLKSGTASVSCLLNNFYPREAKVQWKVDNALQSGNSQESVT |
| | | EQDSKDSTYSLKSTLTLSKADYEKHKVYACEVTHQGLSSPVTKSENRGEC |
| 117 | IL13433-0606 CH3 (3) | GQPREPQVCTLPPSREEMTKNQVSLWCLVKGFYPSDIAVEWESNGQPENNYKTTPP |
| | | VLDSDGSFFLYSKLTVDKSRWQQGNVFSCSVLHEALHSHYTQKSLSLSPGK |
| 118 | IL13433-0606 HC (3) | EVQLVESGGGLVQPGGSLRLSCAASGFTFSSYAMSWVRQAPGKGLEWVASISTGDT |
| | | TYYPDSVKGRFTISRDNAKNSLYLQMNSLRAEDTAVYYCARNEGYYFGLTQWGRGT |
| | | LVTVSSASTKGPSVFPKAPSSKSTSGGTAALGCLVKDYFPEPVTVSWNSGALTSGV |
| | | HTFPAVLQSSGLYSLSSSVTVPSSSLGTQTYICNVNHKPSNTKVDKKVEPKSCDKT |
| | | HTCPPCPAPEAAGAPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNKALPAPIEKT |
| | | ISKAKGQPREPQVCTLPPSREEMTKNQVSLWCLVKGFYPSDIAVEWESNGQPENNY |
| | | KTTPPVLDSDGSFFLYSKLTVDKSRWQQGNVFSCSVLHEALHSHYTQKSLSLSPGK |
| 119 | IL13433-0606 LC (3) | DIQMTQSPSSLSASVGDRVTITCRASQSVDHFGWSLVHWYQQKPGKAPKLLIYRAS |
| | | NLESGVPSRFSGSGSGTDFTLTISSLQPEDFATYYCQQSNEDPWTFGGGTKVEIKR |
| | | TVAAPSVFIFPPSDEQLKSGTASVSCLLNNFYPREAKVQWKVDNALQSGNSQESVT |
| | | EQDSKDSTYSLDSTLTLSKADYEKHKVYACEVTHQGLSSPVTKSENRGEC |
| 120 | IL13433-0607 LC (3) | DIQMTQSPSSLSASVGDRVTITCRASESVDHFGWSLVHWYQQKPGKAPKLLIYRAD |
| | | SLETGVPSRFSGSGSGTDFTLTISSLQPEDFATYYCQQSNEDPWTFGGGTKVEIKR |
| | | TVAAPSVFIFPPSDEQLKSGTASVSCLLNNFYPREAKVQWKVDNALQSGNSQESVT |
| | | EQDSKDSTYSLDSTLTLSKADYEKHKVYACEVTHQGLSSPVTKSENRGEC |
| 121 | IL13433-0717 DFab | DIQMTQSPSSLSASVGDRVTITCKASQNINKHLDWYQQKPGKAPKLLIYFTNNLQT |
| | LC (1)-HC (2) | GVPSRFSGSGSGTDFTLTISSLQPEDFATYYCFQYNQGWTFGGGTKVEIKRTVAAP |
| | | SVFIFPPSDEQLKSGTASVVCLLNNFYPREAKVQWKVDNALQSGNSQESVTEQDSK |
| | | DSTYSLSSTLTLSKADYEKHKVYACEVTHQGLSSPVTKSFNRGECGGGGSEVTLRE |
| | | SGPALVKPTQTLTLTCTFSGFSLSNFGEGLSWIRQPPGKGLEWLAHIYWDDDKRYN |
| | | PSLKSRLTISKDTSRNQVVLTMTNMDPVDTATYYCARRETVFYWYFDVWGQGTTVT |
| | | VSSASTKGPSVFPEAPSSKSTSGGTAALGCLVKDYFPEPVTVSWNSGALTSGVHTF |
| | | PAVLQSSGLYSLGSVVTVPSSSLGTQTYICNVNHKPSNTKVDKKVEPKSCDKTHTC |
| | | PPCPAPEAAGAPSVFLFPPKPKDTLMISRTPEVTCVVVDVSHEDPEVKENWYVDGV |
| | | EVHNAKTKPREEQYNSTYRVVSVLTVLHQDWLNGKEYKCKVSNKALPAPIEKTISK |
| | | AKGQPREPQVYTLPPCREEMTKNQVSLSCAVKGFYPSDIAVEWESNGQPENNYKTT |
| | | PPVLDSDGSFFLVSKLTVDKSRWQQGNVFSCSVLHEALHSHYTQKSLSLSPGK |
| 122 | IL13433-1042 Fd(1) | EVQLVESGGGLVQPGGSLRLSCAASGFTFSSYAMSWVRQAPGKGLEWVASISSGDT |
| | (IL13 VH-CH1 mFd) | TYYPDSVKGRFTISRDNAKNSLYLQMNSLRAEDTAVYYCARNEGYYFGLTLWGQGT |
| | (IL431TSLP-1024, | LVTVSSASTKGPSVFPLAPSSKSTSGGTAALGCLVKDYFPEPVTVSWNSGALTSGV |
| | 1028, 1037) | HTFPAVLQSSGLYSLSSVVTVPSSSLGTQTYICNVNHKPSNTKVDKKVEPKSC |
| 123 | IL13433-1042 HINGE (2) | EPKSCEKTHTCPECP |
| 124 | IL13433-1042 CH3 (2) | GQPREPQVYTLPPSREEMTKNQVSLTCEVKGFYPSDIAVEWESNGQPENNYKTTPP |
| | | VLDSDGSFFLYSKLTVDKSRWQQGNVFSCSVLHEALHSHYTQKSLSLSPGK |
| 125 | IL13433-1042 DFab | DIQMTQSPSSLSASVGDRVTITCKASESVDHFGWSLVHWYQQKPGKAPKLLIYRAS |
| | LC (1)-HC (2) | NLESGVPSRFSGSGSGTDFTLTISSLQPEDFATYYCQQSNEDPWTFGGGTKVEIKR |
| | | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGECGGGGSE |
| | | VTLRESGPALVKPTQTLTLTCTFSGFSLSNFGEGLSWIRQPPGKGLEWLAHIYWDD |
| | | DKRYNPSLKSRLTISKDTSRNQVVLTMTNMDPVDTATYYCARRETVFYWYFDVWGQ |
| | | GTTVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCE |
| | | KTHTCPECPAPEAAGAPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCEVKGFYPSDIAVEWESNGQPEN |
| | | NYKTTPPVLDSDGSFFLYSKLTVDKSRWQQGNVFSCSVLHEALHSHYTQKSLSLSP |
| | | GK |
| 126 | IL13433-1042 HINGE (3) | EPKSCRKTHTCPRCP |
| 127 | IL13433-1042 CH3 (3) | GQPREPQVYTLPPSREEMTKNQVSLTCLVKGFYPSDIAVEWESNGQPENNYKTTPP |
| | | VLDSDGSFFLYSRLTVDKSRWQQGNVFSCSVLHEALHSHYTQKSLSLSPGK |
| 128 | IL13433-1042 SFab | EVQLVESGGGLVQPGGSLRLSCAASGFTESSYWMYWVRQAPGKGLEWVAAITPNAG |
| | HC (3) | EDYYPESVKGRFTISRDNAKNSLYLQMNSLRAEDTAVYYCARGQYYYTKYSLGYWG |
| | | QGTLVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| | | RKTHTCPRCPAPEAAGAPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSRLTVDKSRWQQGNVFSCSVLHEALHSHYTQKSLSLS |
| | | PGK |
| 129 | IL13433-1258 HINGE (2) | EPKSCEKTHTCPPCP |
| 130 | IL13433-1258 DFab | DIQMTQSPSSLSASVGDRVTITCKASESVDHFGWSLVHWYQQKPGKAPKLLIYRAS |
| | LC (1)-HC (2) | NLESGVPSRFSGSGSGTDFTLTISSLQPEDFATYYCQQSNEDPWTFGGGTKVEIKR |
| | | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGECGGGGSE |
| | | VTLRESGPALVKPTQTLTLTCTFSGFSLSNFGEGLSWIRQPPGKGLEWLAHIYWDD |
| | | DKRYNPSLKSRLTISKDTSRNQVVLTMTNMDPVDTATYYCARRETVFYWYFDVWGQ |
| | | GTTVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCE |
| | | KTHTCPPCPAPEAAGAPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCEVKGFYPSDIAVEWESNGQPEN |
| | | NYKTTPPVLDSDGSFFLYSKLTVDKSRWQQGNVFSCSVLHEALHSHYTQKSLSLSP |
| | | GK |
| 131 | IL13433-1258 HINGE (3) | EPKSCRKTHTCPPCP |
| 132 | IL13433-1258 SFab | EVQLVESGGGLVQPGGSLRLSCAASGFTFSSYWMYWVRQAPGKGLEWVAAITPNAG |
| | HC (3) | EDYYPESVKGRFTISRDNAKNSLYLQMNSLRAEDTAVYYCARGQYYYTKYSLGYWG |
| | | QGTLVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| | | RKTHTCPPCPAPEAAGAPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSRLTVDKSRWQQGNVFSCSVLHEALHSHYTQKSLSLS |
| | | PGK |
| 133 | IL13433-1261 DFab | DIQMTQSPSSLSASVGDRVTITCKASESVDHFGWSLVHWYQQKPGKAPKLLIYRAS |
| | LC (1)-HC (2) | NLESGVPSRFSGSGSGTDFTLTISSLQPEDFATYYCQQSNEDPWTFGGGTKVEIKR |
| | | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGECGGGGSE |
| | | VTLRESGPALVKPTQTLTLTCTFSGFSLSNFGEGLSWIRQPPGKGLEWLAHIYWDD |
| | | DKRYNPSLKSRLTISKDTSRNQVVLTMTNMDPVDTATYYCARRETVFYWYFDVWGQ |
| | | GTTVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPEAAGAPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCEVKGFYPSDIAVEWESNGQPEN |
| | | NYKTTPPVLDSDGSFFLYSKLTVDKSRWQQGNVFSCSVLHEALHSHYTQKSLSLSP |
| | | GK |
| 134 | IL13433-1261 SFab | EVQLVESGGGLVQPGGSLRLSCAASGFTFSSYWMYWVRQAPGKGLEWVAAITPNAG |
| | HC (3) | EDYYPESVKGRFTISRDNAKNSLYLQMNSLRAEDTAVYYCARGQYYYTKYSLGYWG |
| | | QGTLVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| | | DKTHTCPPCPAPEAAGAPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSRLTVDKSRWQQGNVFSCSVLHEALHSHYTQKSLSLS |
| | | PGK |
| 135 | IL13433-1270 DFab | DIQMTQSPSSLSASVGDRVTITCKASESVDHFGWSLVHWYQQKPGKAPKLLIYRAS |
| | LC (1)-HC (2) | NLESGVPSRFSGSGSGTDFTLTISSLQPEDFATYYCQQSNEDPWTFGGGTKVEIKR |
| | | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGECGGGGSE |
| | | VTLRESGPALVKPTQTLTLTCTFSGFSLSNFGEGLSWIRQPPGKGLEWLAHIYWDD |
| | | DKRYNPSLKSRLTISKDTSRNQVVLTMTNMDPVDTATYYCARRETVFYWYFDVWGQ |
| | | GTTVTVSSASTKGPSVFPEAPSSKSTSGGTAALGCLVKDYFPEPVTVSWNSGALTS |
| | | GVHTFPAVLQSSGLYSLGSVVTVPSSSLGTQTYICNVNHKPSNTKVDKKVEPKSCD |
| | | KTHTCPPCPAPEAAGAPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIE |
| | | KTISKAKGQPREPQVYTLPPCREEMTKNQVSLSCAVKGFYPSDIAVEWESNGQPEN |
| | | NYKTTPPVLDSDGSFFLVSKLTVDKSRWQQGNVFSCSVLHEALHSHYTQKSLSLSP |
| | | GK |
| 136 | IL13433-1270 DFab | DIQMTQSPSSLSASVGDRVTITCRASQSVDEEGDSYMNWYQQKPGKAPKLLIYAAS |
| | LC (2) | NLESGVPSRFSGSGSGTDFTLTISSLQPEDFATYYCQQSNKDPPTFGGGTKVEIKR |
| | | TVAAPSVFIFPPSDEQLKSGTASVSCLLNNFYPREAKVQWKVDNALQSGNSQESVT |
| | | EQDSKDSTYSLKSTLTLSKADYEKHKVYACEVTHQGLSSPVTKSENRGEC |
| 137 | IL13433-1270 SFab | EVQLVESGGGLVQPGGSLRLSCAASGFTFSSYWMYWVRQAPGKGLEWVAAITPNAG |
| | HC (3) | EDYYPESVKGRFTISRDNAKNSLYLQMNSLRAEDTAVYYCARGQYYYTKYSLGYWG |
| | | QGTLVTVSSASTKGPSVFPKAPSSKSTSGGTAALGCLVKDYFPEPVTVSWNSGALT |
| | | SGVHTFPAVLQSSGLYSLSSSVTVPSSSLGTQTYICNVNHKPSNTKVDKKVEPKSC |
| | | DKTHTCPPCPAPEAAGAPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVCTLPPSREEMTKNQVSLWCLVKGFYPSDIAVEWESNGQPE |
| | | NNYKTTPPVLDSDGSFFLYSKLTVDKSRWQQGNVFSCSVLHEALHSHYTQKSLSLS |
| | | PGK |
| 138 | IL13433-1270 SFab | DIQMTQSPSSLSASVGDRVTITCRASQPIHNHLDWYQQKPGKAPKLLIYFGKNLQE |
| | LC (3) | GVPSRFSGSGSGTDFTLTISSLQPEDFATYYCFQYKKGWSFGGGTKVEIKRTVAAP |
| | | SVFIFPPSDEQLKSGTASVSCLLNNFYPREAKVQWKVDNALQSGNSQESVTEQDSK |
| | | DSTYSLDSTLTLSKADYEKHKVYACEVTHQGLSSPVTKSENRGEC |
| 139 | IL13433-1275 CH3 (2) | GQPREPQVYTLPPSREEMTKNQVSLSCAVKGFYPSDIAVEWESNGQPENNYKTTPP |
| | | VLDSDGSFFLVSKLTVDKSRWQQGNVFSCSVLHEALHSHYTQKSLSLSPGK |
| 140 | IL13433-1275 DFab | DIQMTQSPSSLSASVGDRVTITCKASESVDHFGWSLVHWYQQKPGKAPKLLIYRAS |
| | LC (1)-HC (2) | NLESGVPSRFSGSGSGTDFTLTISSLQPEDFATYYCQQSNEDPWTFGGGTKVEIKR |
| | | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGECGGGGSE |
| | | VTLRESGPALVKPTQTLTLTCTFSGFSLSNFGEGLSWIRQPPGKGLEWLAHIYWDD |
| | | DKRYNPSLKSRLTISKDTSRNQVVLTMTNMDPVDTATYYCARRETVFYWYFDVWGQ |
| | | GTTVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPEAAGAPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLSCAVKGFYPSDIAVEWESNGQPEN |
| | | NYKTTPPVLDSDGSFFLVSKLTVDKSRWQQGNVFSCSVLHEALHSHYTQKSLSLSP |
| | | GK |
| 141 | IL13433-1275 CH3 (3) | GQPREPQVYTLPPSREEMTKNQVSLWCLVKGFYPSDIAVEWESNGQPENNYKTTPP |
| | | VLDSDGSFFLYSKLTVDKSRWQQGNVFSCSVLHEALHSHYTQKSLSLSPGK |
| 142 | IL13433-1275 SFab | EVQLVESGGGLVQPGGSLRLSCAASGFTFSSYWMYWVRQAPGKGLEWVAAITPNAG |
| | HC (3) | EDYYPESVKGRFTISRDNAKNSLYLQMNSLRAEDTAVYYCARGQYYYTKYSLGYWG |
| | | QGTLVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| | | DKTHTCPPCPAPEAAGAPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVYTLPPSREEMTKNQVSLWCLVKGFYPSDIAVEWESNGQPE |
| | | NNYKTTPPVLDSDGSFFLYSKLTVDKSRWQQGNVFSCSVLHEALHSHYTQKSLSLS |
| | | PGK |
| 143 | IL13433-1269 Fd(1) | EVQLVESGGGLVQPGGSLRLSCAASGFTESSYWMYWVRQAPGKGLEWVAAITPNAG |
| | | EDYYPESVKGRFTISRDNAKNSLYLQMNSLRAEDTAVYYCARGQYYYTKYSLGYWG |
| | | QGTLVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| 144 | IL13433-1269 DFab | DIQMTQSPSSLSASVGDRVTITCRASQPIHNHLDWYQQKPGKAPKLLIYFGKNLQE |
| | LC (1)-HC (2) | GVPSRFSGSGSGTDFTLTISSLQPEDFATYYCFQYKKGWSFGGGTKVEIKRTVAAP |
| | | SVFIFPPSDEQLKSGTASVVCLLNNFYPREAKVQWKVDNALQSGNSQESVTEQDSK |
| | | DSTYSLSSTLTLSKADYEKHKVYACEVTHQGLSSPVTKSENRGECGGGGSEVTLRE |
| | | SGPALVKPTQTLTLTCTFSGFSLSNFGEGLSWIRQPPGKGLEWLAHIYWDDDKRYN |
| | | PSLKSRLTISKDTSRNQVVLTMTNMDPVDTATYYCARRETVFYWYFDVWGQGTTVT |
| | | VSSASTKGPSVFPEAPSSKSTSGGTAALGCLVKDYFPEPVTVSWNSGALTSGVHTF |
| | | PAVLQSSGLYSLGSVVTVPSSSLGTQTYICNVNHKPSNTKVDKKVEPKSCDKTHTC |
| | | PPCPAPEAAGAPSVFLFPPKPKDTLMISRTPEVTCVVVDVSHEDPEVKENWYVDGV |
| | | EVHNAKTKPREEQYNSTYRVVSVLTVLHQDWLNGKEYKCKVSNKALPAPIEKTISK |
| | | AKGQPREPQVYTLPPCREEMTKNQVSLSCAVKGFYPSDIAVEWESNGQPENNYKTT |
| | | PPVLDSDGSFFLVSKLTVDKSRWQQGNVFSCSVLHEALHSHYTQKSLSLSPGK |
| 145 | IL13433-0005 DFab | DIQMTQSPSSLSASVGDRVTITCKASQNINKHLDWYQQKPGKAPKLLIYFTNNLQT |
| | LC (1)-HC (2) | GVPSRFSGSGSGTDFTLTISSLQPEDFATYYCFQYNQGWTFGGGTKVEIKRTVAAP |
| | | SVFIFPPSDEQLKSGTASVVCLLNNFYPREAKVQWKVDNALQSGNSQESVTEQDSK |
| | | DSTYSLSSTLTLSKADYEKHKVYACEVTHQGLSSPVTKSFNRGECGGGGSEVQLVE |
| | | SGGGLVQPGGSLRLSCAASGFTESSYAMSWVRQAPGKGLEWVASISSGDTTYYPDS |
| | | VKGRFTISRDNAKNSLYLQMNSLRAEDTAVYYCARNEGYYFGLTLWGQGTLVTVSS |
| | | ASTKGPSVFPKAPSSKSTSGGTAALGCLVKDYFPEPVTVSWNSGALTSGVHTFPAV |
| | | LQSSGLYSLSSSVTVPSSSLGTQTYICNVNHKPSNTKVDKKVEPKSCDKTHTCPPC |
| | | PAPEAAGAPSVFLFPPKPKDTLMISRTPEVTCVVVDVSHEDPEVKENWYVDGVEVH |
| | | NAKTKPREEQYNSTYRVVSVLTVLHQDWLNGKEYKCKVSNKALPAPIEKTISKAKG |
| | | QPREPQVCTLPPSREEMTKNQVSLWCLVKGFYPSDIAVEWESNGQPENNYKTTPPV |
| | | LDSDGSFFLYSKLTVDKSRWQQGNVFSCSVMHEALHNHYTQKSLSLSPGK |
| 146 | IL13433-0005 SFab | EVTLRESGPALVKPTQTLTLTCTFSGFSLSNFGEGLSWIRQPPGKGLEWLAHIYWD |
| | HC (3) | DDKRYNPSLKSRLTISKDTSRNQVVLTMTNMDPVDTATYYCARRETVFYWYFDVWG |
| | | QGTTVTVSSASTKGPSVFPEAPSSKSTSGGTAALGCLVKDYFPEPVTVSWNSGALT |
| | | SGVHTFPAVLQSSGLYSLGSVVTVPSSSLGTQTYICNVNHKPSNTKVDKKVEPKSC |
| | | DKTHTCPPCPAPEAAGAPSVFLFPPKPKDTLMISRTPEVTCVVVDVSHEDPEVKEN |
| | | WYVDGVEVHNAKTKPREEQYNSTYRVVSVLTVLHQDWLNGKEYKCKVSNKALPAPI |
| | | EKTISKAKGQPREPQVYTLPPCREEMTKNQVSLSCAVKGFYPSDIAVEWESNGQPE |
| | | NNYKTTPPVLDSDGSFFLVSKLTVDKSRWQQGNVFSCSVMHEALHNHYTQKSLSLS |
| | | PGK |
| 147 | RRR arm CH3 w/o LS | QPREPQVYTLPPSREEMTKNQVSLTCLVKGFYPSDIAVEWESNGQPENNYKTTPPV |
| | | LDSDGSFFLYSRLTVDKSRWQQGNVFSCSVMHEALHNHYTQKSLSLSPGK |
| 148 | EEE arm CH3 w/o LS | QPREPQVYTLPPSREEMTKNQVSLTCEVKGFYPSDIAVEWESNGQPENNYKTTPPV |
| | | LDSDGSFFLYSKLTVDKSRWQQGNVFSCSVMHEALHNHYTQKSLSLSPGK |
| 149 | IL413TSLP-0001 | QMQLVESGGGVVQPGRSLRLSCAASGFTFRTYGMHWVRQAPGKGLEWVAVIWYDGS |
| | DFab VH-CL (1)-HC (2) | NKHYADSVKGRFTITRDNSKNTLNLQMNSLRAEDTAVYYCARAPQWELVHEAFDIW |
| | IL413TSLP-0001 | GQGTMVTVSSGQPKAAPSVTLFPPSSEELQANKATLVCLISDFYPGAVTVAWKADS |
| | Anti-TSLP/IL-13 dual | SPVKAGVETTTPSKQSNNKYAASSYLSLTPEQWKSHRSYSCQVTHEGSTVEKTVAP |
| | Fab (knob arm | TECSGGGGSEVQLVESGGGLVQPGGSLRLSCAASGFTFSSYAMSWVRQAPGKGLEW |
| | VHCL (1) VHCH (2) | VASISSGDTTYYPDSVKGRFTISRDNAKNSLYLQMNSLRAEDTAVYYCARNEGYYF |
| | | GLTLWGQGTLVTVSSASTKGPSVFPKAPSSKSTSGGTAALGCLVKDYFPEPVTVSW |
| | | NSGALTSGVHTFPAVLQSSGLYSLSSSVTVPSSSLGTQTYICNVNHKPSNTKVDKK |
| | | VEPKSCDKTHTCPPCPAPEAAGAPSVFLFPPKPKDTLMISRTPEVTCVVVDVSHED |
| | | PEVKFNWYVDGVEVHNAKTKPREEQYNSTYRVVSVLTVLHQDWLNGKEYKCKVSNK |
| | | ALPAPIEKTISKAKGQPREPQVCTLPPSREEMTKNQVSLWCLVKGFYPSDIAVEWE |
| | | SNGQPENNYKTTPPVLDSDGSFFLYSKLTVDKSRWQQGNVFSCSVMHEALHNHYTQ |
| | | KSLSLSPGK |
| 150 | IL413TSLP-0001, 0002, | SYVLTQPPSVSVAPGQTARITCGGNNLGSKSVHWYQQKPGQAPVLVVYDDSDRPSW |
| | 0249 | IPERFSGSNSGNTATLTISRGEAGDEADYYCQVWDSSSDHVVFGGGTKLTVLASTK |
| | VL-CH1 (1) | GPSVFPLAPSSKSTSGGTAALGCLVKDYFPEPVTVSWNSGALTSGVHTFPAVLQSS |
| | IL413TSLP-0001 | GLYSLSSVVTVPSSSLGTQTYICNVNHKPSNTKVDKKVEPKSC |
| | Anti-TSLP VLCH1 | |
| 151 | IL413TSLP-0002 | QMQLVESGGGVVQPGRSLRLSCAASGFTFRTYGMHWVRQAPGKGLEWVAVIWYDGS |
| | DFab VH-CL (1)-HC (2) | NKHYADSVKGRFTITRDNSKNTLNLQMNSLRAEDTAVYYCARAPQWELVHEAFDIW |
| | IL413TSLP-0002 | GQGTMVTVSSGQPKAAPSVTLFPPSSEELQANKATLVCLISDFYPGAVTVAWKADS |
| | Anti-TSLP/IL-4 dual | SPVKAGVETTTPSKQSNNKYAASSYLSLTPEQWKSHRSYSCQVTHEGSTVEKTVAP |
| | Fab (hole arm | TECSGGGGSEVTLRESGPALVKPTQTLTLTCTFSGFSLSNFGEGLSWIRQPPGKGL |
| | VHCLVHCH | EWLAHIYWDDDKRYNPSLKSRLTISKDTSRNQVVLTMTNMDPVDTATYYCARRETV |
| | | FYWYFDVWGQGTTVTVSSASTKGPSVFPEAPSSKSTSGGTAALGCLVKDYFPEPVT |
| | | VSWNSGALTSGVHTFPAVLQSSGLYSLGSVVTVPSSSLGTQTYICNVNHKPSNTKV |
| | | DKKVEPKSCDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGKEYKCKV |
| | | SNKALPAPIEKTISKAKGQPREPQVYTLPPCREEMTKNQVSLSCAVKGFYPSDIAV |
| | | EWESNGQPENNYKTTPPVLDSDGSFFLVSKLTVDKSRWQQGNVFSCSVMHEALHNH |
| | | YTQKSLSLSPGK |
| 152 | IL413TSLP-0003 | QMQLVESGGGVVQPGRSLRLSCAASGFTFRTYGMHWVRQAPGKGLEWVAVIWYDGS |
| | DFab VH-CH1 (1)-HC (2) | NKHYADSVKGRFTITRDNSKNTLNLQMNSLRAEDTAVYYCARAPQWELVHEAFDIW |
| | Anti-TSLP/IL-13 dual | GQGTMVTVSSASTKGPSVFPLAPSSKSTSGGTAALGCLVKDYFPEPVTVSWNSGAL |
| | Fab (knob arm | TSGVHTFPAVLQSSGLYSLSSVVTVPSSSLGTQTYICNVNHKPSNTKVDKKVEPKS |
| | VH1CH1VHCH (2) | CGGGGSEVQLVESGGGLVQPGGSLRLSCAASGFTFSSYAMSWVRQAPGKGLEWVAS |
| | | ISSGDTTYYPDSVKGRFTISRDNAKNSLYLQMNSLRAEDTAVYYCARNEGYYFGLT |
| | | LWGQGTLVTVSSASTKGPSVFPKAPSSKSTSGGTAALGCLVKDYFPEPVTVSWNSG |
| | | ALTSGVHTFPAVLQSSGLYSLSSSVTVPSSSLGTQTYICNVNHKPSNTKVDKKVEP |
| | | KSCDKTHTCPPCPAPEAAGAPSVFLFPPKPKDTLMISRTPEVTCVVVDVSHEDPEV |
| | | KFNWYVDGVEVHNAKTKPREEQYNSTYRVVSVLTVLHQDWLNGKEYKCKVSNKALP |
| | | APIEKTISKAKGQPREPQVCTLPPSREEMTKNQVSLWCLVKGFYPSDIAVEWESNG |
| | | QPENNYKTTPPVLDSDGSFFLYSKLTVDKSRWQQGNVFSCSVMHEALHNHYTQKSL |
| | | SLSPGK |
| 153 | IL413TSLP-0004 | QMQLVESGGGVVQPGRSLRLSCAASGFTFRTYGMHWVRQAPGKGLEWVAVIWYDGS |
| | DFab VH-CH1 (1)-HC (2) | NKHYADSVKGRFTITRDNSKNTLNLQMNSLRAEDTAVYYCARAPQWELVHEAFDIW |
| | Anti-TSLP/IL-4 dual | GQGTMVTVSSASTKGPSVFPLAPSSKSTSGGTAALGCLVKDYFPEPVTVSWNSGAL |
| | Fab (hole arm | TSGVHTFPAVLQSSGLYSLSSVVTVPSSSLGTQTYICNVNHKPSNTKVDKKVEPKS |
| | VH1CH1VHCH (2) | CGGGGSEVTLRESGPALVKPTQTLTLTCTFSGFSLSNFGEGLSWIRQPPGKGLEWL |
| | | AHIYWDDDKRYNPSLKSRLTISKDTSRNQVVLTMTNMDPVDTATYYCARRETVFYW |
| | | YFDVWGQGTTVTVSSASTKGPSVFPEAPSSKSTSGGTAALGCLVKDYFPEPVTVSW |
| | | NSGALTSGVHTFPAVLQSSGLYSLGSVVTVPSSSLGTQTYICNVNHKPSNTKVDKK |
| | | VEPKSCDKTHTCPPCPAPEAAGAPSVFLFPPKPKDTLMISRTPEVTCVVVDVSHED |
| | | PEVKFNWYVDGVEVHNAKTKPREEQYNSTYRVVSVLTVLHQDWLNGKEYKCKVSNK |
| | | ALPAPIEKTISKAKGQPREPQVYTLPPCREEMTKNQVSLSCAVKGFYPSDIAVEWE |
| | | SNGQPENNYKTTPPVLDSDGSFFLVSKLTVDKSRWQQGNVFSCSVMHEALHNHYTQ |
| | | KSLSLSPGK |
| 154 | IL413TSLP-0007 | SYVLTQPPSVSVAPGQTARITCGGNNLGSKSVHWYQQKPGQAPVLVVYDDSDRPSW |
| | DFab VL-CH1 (1)-HC (2) | IPERFSGSNSGNTATLTISRGEAGDEADYYCQVWDSSSDHVVFGGGTKLTVLASTK |
| | Anti-TSLP/IL-13 dual | GPSVFPLAPSSKSTSGGTAALGCLVKDYFPEPVTVSWNSGALTSGVHTFPAVLQSS |
| | Fab (knob arm VLCH1- | GLYSLSSVVTVPSSSLGTQTYICNVNHKPSNTKVDKKVEPKSCGGGGSEVQLVESG |
| | VHCH (2) | GGLVQPGGSLRLSCAASGFTFSSYAMSWVRQAPGKGLEWVASISSGDTTYYPDSVK |
| | | GRFTISRDNAKNSLYLQMNSLRAEDTAVYYCARNEGYYFGLTLWGQGTLVTVSSAS |
| | | TKGPSVFPKAPSSKSTSGGTAALGCLVKDYFPEPVTVSWNSGALTSGVHTFPAVLQ |
| | | SSGLYSLSSSVTVPSSSLGTQTYICNVNHKPSNTKVDKKVEPKSCDKTHTCPPCPA |
| | | PEAAGAPSVFLFPPKPKDTLMISRTPEVTCVVVDVSHEDPEVKENWYVDGVEVHNA |
| | | KTKPREEQYNSTYRVVSVLTVLHQDWLNGKEYKCKVSNKALPAPIEKTISKAKGQP |
| | | REPQVCTLPPSREEMTKNQVSLWCLVKGFYPSDIAVEWESNGQPENNYKTTPPVLD |
| | | SDGSFFLYSKLTVDKSRWQQGNVFSCSVMHEALHNHYTQKSLSLSPGK |
| 155 | IL413TSLP-0007 | QMQLVESGGGVVQPGRSLRLSCAASGFTFRTYGMHWVRQAPGKGLEWVAVIWYDGS |
| | VH1-CL (1) | NKHYADSVKGRFTITRDNSKNTLNLQMNSLRAEDTAVYYCARAPQWELVHEAFDIW |
| | Anti-TSLP VHCL (1) | GQGTMVTVSSGQPKAAPSVTLFPPSSEELQANKATLVCLISDFYPGAVTVAWKADS |
| | | SPVKAGVETTTPSKQSNNKYAASSYLSLTPEQWKSHRSYSCQVTHEGSTVEKTVAP |
| | | TECS |
| 156 | IL413TSLP-0008 | SYVLTQPPSVSVAPGQTARITCGGNNLGSKSVHWYQQKPGQAPVLVVYDDSDRPSW |
| | DFab VL-CH1 (1)-HC (2) | IPERFSGSNSGNTATLTISRGEAGDEADYYCQVWDSSSDHVVFGGGTKLTVLASTK |
| | Anti-TSLP/IL-4 dual | GPSVFPLAPSSKSTSGGTAALGCLVKDYFPEPVTVSWNSGALTSGVHTFPAVLQSS |
| | Fab (hole arm | GLYSLSSVVTVPSSSLGTQTYICNVNHKPSNTKVDKKVEPKSCGGGGSEVTLRESG |
| | VLCH1 (1)-VHCH (2) | PALVKPTQTLTLTCTFSGFSLSNFGEGLSWIRQPPGKGLEWLAHIYWDDDKRYNPS |
| | | LKSRLTISKDTSRNQVVLTMTNMDPVDTATYYCARRETVFYWYFDVWGQGTTVTVS |
| | | SASTKGPSVFPEAPSSKSTSGGTAALGCLVKDYFPEPVTVSWNSGALTSGVHTFPA |
| | | VLQSSGLYSLGSVVTVPSSSLGTQTYICNVNHKPSNTKVDKKVEPKSCDKTHTCPP |
| | | CPAPEAAGAPSVFLFPPKPKDTLMISRTPEVTCVVVDVSHEDPEVKENWYVDGVEV |
| | | HNAKTKPREEQYNSTYRVVSVLTVLHQDWLNGKEYKCKVSNKALPAPIEKTISKAK |
| | | GQPREPQVYTLPPCREEMTKNQVSLSCAVKGFYPSDIAVEWESNGQPENNYKTTPP |
| | | VLDSDGSFFLVSKLTVDKSRWQQGNVFSCSVMHEALHNHYTQKSLSLSPGK |
| 157 | IL413TSLP-0248 | SYVLTQPPSVSVAPGQTARITCGGNNLGSKSVHWYQQKPGQAPVLVVYDDSDRPSW |
| | DFab LC (1)-HC (2) | IPERFSGSNSGNTATLTISRGEAGDEADYYCQVWDSSSDHVVFGGGTKLTVLGQPK |
| | Anti-TSLP/IL-4 dual | AAPSVTLFPPSSEELQANKATLVCLISDFYPGAVTVAWKADSSPVKAGVETTTPSK |
| | Fab (hole arm | QSNNKYAASSYLSLTPEQWKSHRSYSCQVTHEGSTVEKTVAPTECSGGGGSEVTLR |
| | | ESGPALVKPTQTLTLTCTFSGFSLSNFGEGLSWIRQPPGKGLEWLAHIYWDDDKRY |
| | | NPSLKSRLTISKDTSRNQVVLTMTNMDPVDTATYYCARRETVFYWYFDVWGQGTTV |
| | | TVSSASTKGPSVFPEAPSSKSTSGGTAALGCLVKDYFPEPVTVSWNSGALTSGVHT |
| | | FPAVLQSSGLYSLGSVVTVPSSSLGTQTYICNVNHKPSNTKVDKKVEPKSCDKTHT |
| | | CPPCPAPEAAGAPSVFLFPPKPKDTLMISRTPEVTCVVVDVSHEDPEVKENWYVDG |
| | | VEVHNAKTKPREEQYNSTYRVVSVLTVLHQDWLNGKEYKCKVSNKALPAPIEKTIS |
| | | KAKGQPREPQVYTLPPCREEMTKNQVSLSCAVKGFYPSDIAVEWESNGQPENNYKT |
| | | TPPVLDSDGSFFLVSKLTVDKSRWQQGNVFSCSVMHEALHNHYTQKSLSLSPGK |
| 158 | IL413TSLP-0248, 0250 | QMQLVESGGGVVQPGRSLRLSCAASGFTFRTYGMHWVRQAPGKGLEWVAVIWYDGS |
| | Modified Fd (1) | NKHYADSVKGRFTISRDNSKNTLYLQMNSLRAEDTAVYYCARAPQWELVHEAFDIW |
| | Anti-TSLP VHCH1 | GQGTMVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKKVEPKS |
| | | C |
| 159 | IL413TSLP-0249 | QMQLVESGGGVVQPGRSLRLSCAASGFTFRTYGMHWVRQAPGKGLEWVAVIWYDGS |
| | DFab VH-CL (1)-HC (2) | NKHYADSVKGRFTISRDNSKNTLYLQMNSLRAEDTAVYYCARAPQWELVHEAFDIW |
| | Anti-TSLP/IL-4 dual | GQGTMVTVSSGQPKAAPSVTLFPPSSEELQANKATLVCLISDFYPGAVTVAWKADS |
| | Fab (hole arm | SPVKAGVETTTPSKQSNNKYAASSYLSLTPEQWKSHRSYSCQVTHEGSTVEKTVAP |
| | VHCL (1) VHCH (2) | TECSGGGGSEVTLRESGPALVKPTQTLTLTCTFSGFSLSNFGEGLSWIRQPPGKGL |
| | | EWLAHIYWDDDKRYNPSLKSRLTISKDTSRNQVVLTMTNMDPVDTATYYCARRETV |
| | | FYWYFDVWGQGTTVTVSSASTKGPSVFPEAPSSKSTSGGTAALGCLVKDYFPEPVT |
| | | VSWNSGALTSGVHTFPAVLQSSGLYSLGSVVTVPSSSLGTQTYICNVNHKPSNTKV |
| | | DKKVEPKSCDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGKEYKCKV |
| | | SNKALPAPIEKTISKAKGQPREPQVYTLPPCREEMTKNQVSLSCAVKGFYPSDIAV |
| | | EWESNGQPENNYKTTPPVLDSDGSFFLVSKLTVDKSRWQQGNVFSCSVMHEALHNH |
| | | YTQKSLSLSPGK |
| 160 | IL413TSLP-0250 | SYVLTQPPSVSVAPGQTARITCGGNNLGSKSVHWYQQKPGQAPVLVVYDDSDRPSW |
| | DFab LC (1)-HC (2) | IPERFSGSNSGNTATLTISRGEAGDEADYYCQVWDSSSDHVVFGGGTKLTVLGQPK |
| | Anti-TSLP/IL-13 dual | AAPSVTLFPPSSEELQANKATLVCLISDFYPGAVTVAWKADSSPVKAGVETTTPSK |
| | Fab knob arm | QSNNKYAASSYLSLTPEQWKSHRSYSCQVTHEGSTVEKTVAPTECSGGGGSEVQLV |
| | | ESGGGLVQPGGSLRLSCAASGFTFSSYAMSWVRQAPGKGLEWVASISSGDTTYYPD |
| | | SVKGRFTISRDNAKNSLYLQMNSLRAEDTAVYYCARNEGYYFGLTLWGQGTLVTVS |
| | | SASTKGPSVFPKAPSSKSTSGGTAALGCLVKDYFPEPVTVSWNSGALTSGVHTFPA |
| | | VLQSSGLYSLSSSVTVPSSSLGTQTYICNVNHKPSNTKVDKKVEPKSCDKTHTCPP |
| | | CPAPEAAGAPSVFLFPPKPKDTLMISRTPEVTCVVVDVSHEDPEVKENWYVDGVEV |
| | | HNAKTKPREEQYNSTYRVVSVLTVLHQDWLNGKEYKCKVSNKALPAPIEKTISKAK |
| | | GQPREPQVCTLPPSREEMTKNQVSLWCLVKGFYPSDIAVEWESNGQPENNYKTTPP |
| | | VLDSDGSFFLYSKLTVDKSRWQQGNVFSCSVMHEALHNHYTQKSLSLSPGK |
| 161 | IL413TSLP-0251, 0252 | QMQLVESGGGVVQPGRSLRLSCAASGFTFRTYGMHWVRQAPGKGLEWVAVIWYDGS |
| | SFab HC (3) | NKHYADSVKGRFTISRDNSKNTLYLQMNSLRAEDTAVYYCARAPQWELVHEAFDIW |
| | Anti-TSLP single Fab | GQGTMVTVSSASTKGPSVFPLAPSSKSTSGGTAALGCLVKDYFPEPVTVSWNSGAL |
| | heavy chain (RRR arm) | TSGVHTFPAVLQSSGLYSLSSVVTVPSSSLGTQTYICNVNHKPSNTKVDKKVEPKS |
| | | CRKTHTCPRCPAPEAAGAPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVEWESNGQP |
| | | ENNYKTTPPVLDSDGSFFLYSRLTVDKSRWQQGNVFSCSVMHEALHNHYTQKSLSL |
| | | SPGK |
| 162 | IL413TSLP-0251 | EVQLVESGGGLVQPGGSLRLSCAASGFTFSSYAMSWVRQAPGKGLEWVASISSGDT |
| | DFab VH-CL (1)-HC (2) | TYYPDSVKGRFTISRDNAKNSLYLQMNSLRAEDTAVYYCARNEGYYFGLTLWGQGT |
| | Anti-IL-13/IL-4 dual | LVTVSSRTVAAPSVFIFPPSDEQLKSGTASVVCLLNNFYPREAKVQWKVDNALQSG |
| | Fab (EEE arm) | NSQESVTEQDSKDSTYSLSSTLTLSKADYEKHKVYACEVTHQGLSSPVTKSENRGE |
| | | CGGGGSEVTLRESGPALVKPTQTLTLTCTFSGFSLSNFGEGLSWIRQPPGKGLEWL |
| | | AHIYWDDDKRYNPSLKSRLTISKDTSRNQVVLTMTNMDPVDTATYYCARRETVFYW |
| | | YFDVWGQGTTVTVSSASTKGPSVFPLAPSSKSTSGGTAALGCLVKDYFPEPVTVSW |
| | | NSGALTSGVHTFPAVLQSSGLYSLSSVVTVPSSSLGTQTYICNVNHKPSNTKVDKK |
| | | VEPKSCEKTHTCPECPAPEAAGAPSVFLFPPKPKDTLMISRTPEVTCVVVDVSHED |
| | | PEVKFNWYVDGVEVHNAKTKPREEQYNSTYRVVSVLTVLHQDWLNGKEYKCKVSNK |
| | | ALPAPIEKTISKAKGQPREPQVYTLPPSREEMTKNQVSLTCEVKGFYPSDIAVEWE |
| | | SNGQPENNYKTTPPVLDSDGSFFLYSKLTVDKSRWQQGNVFSCSVMHEALHNHYTQ |
| | | KSLSLSPGK |
| 163 | IL413TSLP-0251 | DIQMTQSPSSLSASVGDRVTITCKASESVDHFGWSLVHWYQQKPGKAPKLLIYRAS |
| | VL-CH1 (1) | NLESGVPSRFSGSGSGTDFTLTISSLQPEDFATYYCQQSNEDPWTFGGGTKVEIKA |
| | Anti-IL-13 VKCH1 | STKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSC |
| 164 | IL413TSLP-0252 | DIQMTQSPSSLSASVGDRVTITCKASESVDHFGWSLVHWYQQKPGKAPKLLIYRAS |
| | DFab LC (1)-HC (2) | NLESGVPSRFSGSGSGTDFTLTISSLQPEDFATYYCQQSNEDPWTFGGGTKVEIKR |
| | Anti-IL-13/4 dual Fab | TVAAPSVFIFPPSDEQLKSGTASVVCLLNNFYPREAKVQWKVDNALQSGNSQESVT |
| | EE arm | EQDSKDSTYSLSSTLTLSKADYEKHKVYACEVTHQGLSSPVTKSFNRGECGGGGSE |
| | | VTLRESGPALVKPTQTLTLTCTFSGFSLSNFGEGLSWIRQPPGKGLEWLAHIYWDD |
| | | DKRYNPSLKSRLTISKDTSRNQVVLTMTNMDPVDTATYYCARRETVFYWYFDVWGQ |
| | | GTTVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCE |
| | | KTHTCPECPAPEAAGAPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCEVKGFYPSDIAVEWESNGQPEN |
| | | NYKTTPPVLDSDGSFFLYSKLTVDKSRWQQGNVFSCSVMHEALHNHYTQKSLSLSP |
| | | GK |
| 165 | IL413TSLP-1024 | QMQLVESGGGVVQPGRSLRLSCAASGFTFRTYGMHWVRQAPGKGLEWVAVIWYDGS |
| | IL413TSLP-1028 | NKHYADSVKGRFTISRDNSKNTLYLQMNSLRAEDTAVYYCARAPQWYLVHEAFDIW |
| | IL413TSLP-1037 | GQGTMVTVSSASTKGPSVFPLAPSSKSTSGGTAALGCLVKDYFPEPVTVSWNSGAL |
| | SFab HC (3) | TSGVHTFPAVLQSSGLYSLSSVVTVPSSSLGTQTYICNVNHKPSNTKVDKKVEPKS |
| | Anti-TSLP single Fab | CRKTHTCPPCPAPEAAGAPSVFLFPPKPKDTLMISRTPEVTCVVVDVSHEDPEVKF |
| | heavy chain (RR arm) | NWYVDGVEVHNAKTKPREEQYNSTYRVVSVLTVLHQDWLNGKEYKCKVSNKALPAP |
| | | IEKTISKAKGQPREPQVYTLPPSREEMTKNQVSLTCLVKGFYPSDIAVEWESNGQP |
| | | ENNYKTTPPVLDSDGSFFLYSRLTVDKSRWQQGNVFSCSVLHEALHSHYTQKSLSL |
| | | SPGK |
| 166 | P40-0003 CDRH1 | GYSFTTYWLG |
| 167 | P40-0003 CDRH2 | IMSPVDSDIRYSPSFQG |
| 168 | P40-0003 CDRH3 | RRPGQGYFDF |
| 169 | P40-0003 VH | EVQLVQSGAEVKKPGESLKISCKGSGYSFTTYWLGWVRQMPGKGLDWIGIMSPVDS |
| | | DIRYSPSFQGQVTMSVDKSITTAYLQWNSLKASDTAMYYCARRRPGQGYFDFWGQG |
| | | TLVTVSS |
| 170 | P40-0003 HC | EVQLVQSGAEVKKPGESLKISCKGSGYSFTTYWLGWVRQMPGKGLDWIGIMSPVDS |
| | | DIRYSPSFQGQVTMSVDKSITTAYLQWNSLKASDTAMYYCARRRPGQGYFDFWGQG |
| | | TLVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEPKSCDK |
| | | THTCPPCPAPEAAGAPSVFLFPPKPKDTLMISRTPEVTCVVVDVSHEDPEVKENWY |
| | | VDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENN |
| | | YKTTPPVLDSDGSFFLYSKLTVDKSRWQQGNVFSCSVMHEALHNHYTQKSLSLSPG |
| 171 | P40-0003 CDRL1 | RASQGISSWLA |
| 172 | P40-0003 CDRL2 | AASSLQS |
| 173 | P40-0003 CDRL3 | QQYNIYPYT |
| 174 | P40-0003 JK | FGQGTKLEIK |
| 175 | P40-0003 VL | DIQMTQSPSSLSASVGDRVTITCRASQGISSWLAWYQQKPEKAPKSLIYAASSLQS |
| | | GVPSRFSGSGSGTDFTLTISSLQPEDFATYYCQQYNIYPYTFGQGTKLEIK |
| 176 | P40-0003 LC | DIQMTQSPSSLSASVGDRVTITCRASQGISSWLAWYQQKPEKAPKSLIYAASSLQS |
| | | GVPSRFSGSGSGTDFTLTISSLQPEDFATYYCQQYNIYPYTFGQGTKLEIKRTVAA |
| | | PSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSENRGEC |
| 177 | IL413p40-0043 Fd(1) | EVQLVQSGAEVKKPGESLKISCKGSGYSFTTYWLGWVRQMPGKGLDWIGIMSPVDS |
| | IL413p40-0044 Fd (1) | DIRYSPSFQGQVTMSVDKSITTAYLQWNSLKASDTAMYYCARRRPGQGYFDFWGQG |
| | | TLVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEPKSC |
| 178 | IL413p40-0043 DFab | DIQMTQSPSSLSASVGDRVTITCRASQGISSWLAWYQQKPEKAPKSLIYAASSLQS |
| | LC (1)-HC (2) | GVPSRFSGSGSGTDFTLTISSLQPEDFATYYCQQYNIYPYTFGQGTKLEIKRTVAA |
| | | PSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGECGGGGSEVQLV |
| | | ESGGGLVQPGGSLRLSCAASGFTFSSYAMSWVRQAPGKGLEWVASISSGDTTYYPD |
| | | SVKGRFTISRDNAKNSLYLQMNSLRAEDTAVYYCARNEGYYFGLTLWGQGTLVTVS |
| | | SASTKGPSVFPKAPSSKSTSGGTAALGCLVKDYFPEPVTVSWNSGALTSGVHTFPA |
| | | VLQSSGLYSLSSSVTVPSSSLGTQTYICNVNHKPSNTKVDKKVEPKSCDKTHTCPP |
| | | CPAPEAAGAPSVFLFPPKPKDTLMISRTPEVTCVVVDVSHEDPEVKENWYVDGVEV |
| | | HNAKTKPREEQYNSTYRVVSVLTVLHQDWLNGKEYKCKVSNKALPAPIEKTISKAK |
| | | GQPREPQVCTLPPSREEMTKNQVSLWCLVKGFYPSDIAVEWESNGQPENNYKTTPP |
| | | VLDSDGSFFLYSKLTVDKSRWQQGNVFSCSVMHEALHNHYTQKSLSLSPGK |
| 179 | IL413p40-0044 DFab LC | DIQMTQSPSSLSASVGDRVTITCRASQGISSWLAWYQQKPEKAPKSLIYAASSLQS |
| | (1)-HC (2) | GVPSRFSGSGSGTDFTLTISSLQPEDFATYYCQQYNIYPYTFGQGTKLEIKRTVAA |
| | | PSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSENRGECGGGGSEVTLR |
| | | ESGPALVKPTQTLTLTCTFSGFSLSNFGEGLSWIRQPPGKGLEWLAHIYWDDDKRY |
| | | NPSLKSRLTISKDTSRNQVVLTMTNMDPVDTATYYCARRETVFYWYFDVWGQGTTV |
| | | TVSSASTKGPSVFPEAPSSKSTSGGTAALGCLVKDYFPEPVTVSWNSGALTSGVHT |
| | | FPAVLQSSGLYSLGSVVTVPSSSLGTQTYICNVNHKPSNTKVDKKVEPKSCDKTHT |
| | | CPPCPAPEAAGAPSVELFPPKPKDTLMISRTPEVTCVVVDVSHEDPEVKENWYVDG |
| | | VEVHNAKTKPREEQYNSTYRVVSVLTVLHQDWLNGKEYKCKVSNKALPAPIEKTIS |
| | | KAKGQPREPQVYTLPPCREEMTKNQVSLSCAVKGFYPSDIAVEWESNGQPENNYKT |
| | | TPPVLDSDGSFFLVSKLTVDKSRWQQGNVFSCSVMHEALHNHYTQKSLSLSPGK |
| 180 | IL413p40-0642 DFab LC | DIQMTQSPSSLSASVGDRVTITCKASESVDHFGWSLVHWYQQKPGKAPKLLIYRAS |
| | (1)-HC (2) | NLESGVPSRFSGSGSGTDFTLTISSLQPEDFATYYCQQSNEDPWTFGGGTKVEIKR |
| | | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGECGGGGSE |
| | | VTLRESGPALVKPTQTLTLTCTFSGFSLSNFGEGLSWIRQPPGKGLEWLAHIYWDD |
| | | DKRYNPSLKSRLTISKDTSRNQVVLTMTNMDPVDTATYYCARRETVFYWYFDVWGQ |
| | | GTTVTVSSASTKGPSVFPEAPSSKSTSGGTAALGCLVKDYFPEPVTVSWNSGALTS |
| | | GVHTFPAVLQSSGLYSLGSVVTVPSSSLGTQTYICNVNHKPSNTKVDKKVEPKSCD |
| | | KTHTCPPCPAPEAAGAPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIE |
| | | KTISKAKGQPREPQVYTLPPCREEMTKNQVSLSCAVKGFYPSDIAVEWESNGQPEN |
| | | NYKTTPPVLDSDGSFFLVSKLTVDKSRWQQGNVFSCSVMHEALHNHYTQKSLSLSP |
| | | GK |
| 181 | IL413p40-0642 HC (3) | EVQLVQSGAEVKKPGESLKISCKGSGYSFTTYWLGWVRQMPGKGLDWIGIMSPVDS |
| | | DIRYSPSFQGQVTMSVDKSITTAYLQWNSLKASDTAMYYCARRRPGQGYFDFWGQG |
| | | TLVTVSSASTKGPSVFPKAPSSKSTSGGTAALGCLVKDYFPEPVTVSWNSGALTSG |
| | | VHTFPAVLQSSGLYSLSSSVTVPSSSLGTQTYICNVNHKPSNTKVDKKVEPKSCDK |
| | | THTCPPCPAPEAAGAPSVFLFPPKPKDTLMISRTPEVTCVVVDVSHEDPEVKENWY |
| | | VDGVEVHNAKTKPREEQYNSTYRVVSVLTVLHQDWLNGKEYKCKVSNKALPAPIEK |
| | | TISKAKGQPREPQVCTLPPSREEMTKNQVSLWCLVKGFYPSDIAVEWESNGQPENN |
| | | YKTTPPVLDSDGSFFLYSKLTVDKSRWQQGNVFSCSVMHEALHNHYTQKSLSLSPG |
| | | K |
| 182 | IL413p40-0642 LC (3) | DIQMTQSPSSLSASVGDRVTITCRASQGISSWLAWYQQKPEKAPKSLIYAASSLQS |
| | | GVPSRFSGSGSGTDFTLTISSLQPEDFATYYCQQYNIYPYTFGQGTKLEIKTVAAP |
| | | SVFIFPPSDEQLKSGTASVSCLLNNFYPREAKVQWKVDNALQSGNSQESVTEQDSK |
| | | DSTYSLDSTLTLSKADYEKHKVYACEVTHQGLSSPVTKSENRGEC |
| 183 | IL413p40-0648 DFab | DIQMTQSPSSLSASVGDRVTITCRASQGISSWLAWYQQKPEKAPKSLIYAASSLQS |
| | LC (1)-HC (2) | GVPSRFSGSGSGTDFTLTISSLQPEDFATYYCQQYNIYPYTFGQGTKLEIKRTVAA |
| | | PSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGECGGGGSEVTLR |
| | | ESGPALVKPTQTLTLTCTFSGFSLSNFGEGLSWIRQPPGKGLEWLAHIYWDDDKRY |
| | | STSLKTRLTISKDTSRNQVVLTMTNMDPVDTATYYCARRETVFYWYFDVWGQGTTV |
| | | TVSSASTKGPSVFPEAPSSKSTSGGTAALGCLVKDYFPEPVTVSWNSGALTSGVHT |
| | | FPAVLQSSGLYSLGSVVTVPSSSLGTQTYICNVNHKPSNTKVDKKVEPKSCDKTHT |
| | | CPPCPAPEAAGAPSVELFPPKPKDTLMISRTPEVTCVVVDVSHEDPEVKENWYVDG |
| | | VEVHNAKTKPREEQYNSTYRVVSVLTVLHQDWLNGKEYKCKVSNKALPAPIEKTIS |
| | | KAKGQPREPQVYTLPPCREEMTKNQVSLSCAVKGFYPSDIAVEWESNGQPENNYKT |
| | | TPPVLDSDGSFFLVSKLTVDKSRWQQGNVFSCSVLHEALHSHYTQKSLSLSPGK |
| 184 | IL413p40-0648 LC (3) | DIQMTQSPSSLSASVGDRVTITCRASESVDHFGWSLVHWYQQKPGKAPKLLIYRAD |
| | | SLETGVPSRFSGSGSGTDFTLTISSLQPEDFATYYCQQSNEDPWTFGGGTKVEIKR |
| | | TVAAPSVFIFPPSDEQLKSGTASVSCLLNNFYPREAKVQWKVDNALQSGNSQESVT |
| | | EQDSKDSTYSLDSTLTLSKADYEKHKVYACEVTHQGLSSPVTKSENRGEC |
| 185 | IL413p40-0698 SFab | EVQLVQSGAEVKKPGESLKISCKGSGYSFTTYWLGWVRQMPGKGLDWIGIMSPVDS |
| | HC (3) | DIRYSPSFQGQVTMSVDKSITTAYLQWNSLKASDTAMYYCARRRPGQGYFDFWGQG |
| | | TLVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEPKSCRK |
| | | THTCPRCPAPEAAGAPSVFLFPPKPKDTLMISRTPEVTCVVVDVSHEDPEVKENWY |
| | | VDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENN |
| | | YKTTPPVLDSDGSFFLYSRLTVDKSRWQQGNVFSCSVLHEALHSHYTQKSLSLSPG |
| | | K |
| 186 | IL413p40-0705 SFab | EVQLVQSGAEVKKPGESLKISCKGSGYSFTTYWLGWVRQMPGKGLDWIGIMSPVDS |
| | HC (3) | DIRYSPSFQGQVTMSVDKSITTAYLQWNSLKASDTAMYYCARRRPGQGYFDFWGQG |
| | | TLVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEPKSCRK |
| | | THTCPPCPAPEAAGAPSVFLFPPKPKDTLMISRTPEVTCVVVDVSHEDPEVKENWY |
| | | VDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENN |
| | | YKTTPPVLDSDGSFFLYSRLTVDKSRWQQGNVFSCSVLHEALHSHYTQKSLSLSPG |
| | | K |
| 187 | IL13-mFd (nucleic | GAGGTGCAGCTGGTGGAGTCAGGGGGAGGCTTGGTGCAACCTGGAGGGTCCCTGAG |
| | acid) | ACTCTCCTGTGCAGCCTCTGGATTCACTTTCAGTAGCTATGCCATGTCTTGGGTTC |
| | | GTCAGGCTCCAGGGAAGGGGCTGGAGTGGGTCGCATCCATTAGTAGTGGTGACACC |
| | | ACCTACTATCCAGACAGCGTGAAGGGCCGATTCACCATCTCCAGAGATAATGCCAA |
| | | GAACAGCCTGTACCTGCAAATGAACAGTCTGAGGGCTGAGGACACCGCCGTGTATT |
| | | ACTGTGCACGAAACGAGGGTTACTACTTCGGACTGACCCTGTGGGGCCAAGGGACC |
| | | CTGGTCACCGTCTCCTCTGCGTCGACCAAGGGCCCATCGGTCTTCCCCCTGGCACC |
| | | CTCCTCCAAGAGCACCTCTGGGGGCACAGCGGCCCTGGGCTGCCTGGTCAAGGACT |
| | | ACTTCCCCGAACCGGTGACGGTGTCGTGGAACTCAGGCGCCCTGACCAGCGGCGTG |
| | | CACACCTTCCCGGCTGTCCTACAGTCCTCAGGACTCTACTCCCTCAGCAGCGTGGT |
| | | GACCGTGCCCTCCAGCAGCTTGGGCACCCAGACCTACATCTGCAACGTGAATCACA |
| | | AGCCCAGCAACACCAAGGTGGACAAGAAAGTTGAGCCCAAATCTTGT |
| 188 | DFab IL13-LC-IL4-HC | GACATCCAGATGACCCAGTCTCCATCCTCCCTGTCTGCATCTGTGGGAGACAGAGT |
| | (nucleic acid) | CACCATCACTTGCAAAGCCAGTGAAAGTGTTGATCACTTTGGTTGGTCTCTCGTGC |
| | | ACTGGTATCAGCAGAAACCAGGGAAAGCTCCTAAGCTCCTGATCTATCGTGCATCC |
| | | AACCTGGAATCTGGCGTCCCATCAAGGTTCAGTGGCAGTGGATCTGGCACAGATTT |
| | | CACTCTCACCATCAGCAGTCTGCAACCTGAAGATTTTGCAACTTACTACTGTCAGC |
| | | AAAGTAATGAAGATCCCTGGACCTTCGGCGGAGGGACCAAGGTGGAGATCAAACGT |
| | | ACGGTGGCTGCACCATCTGTCTTCATCTTCCCGCCATCTGATGAGCAGTTGAAATC |
| | | TGGAACTGCCTCTGTTGTGTGCCTGCTGAATAACTTCTATCCCAGAGAGGCCAAAG |
| | | TACAGTGGAAGGTGGATAACGCCCTCCAATCGGGTAACTCCCAGGAGAGTGTCACA |
| | | GAGCAGGACAGCAAGGACAGCACCTACAGCCTCAGCAGCACCCTGACGCTGAGCAA |
| | | AGCAGACTACGAGAAACACAAAGTCTACGCCTGCGAAGTCACCCATCAGGGCCTGA |
| | | GCTCGCCCGTCACAAAGAGCTTCAACAGGGGAGAGTGTGGCGGTGGCGGGTCCGAG |
| | | GTCACACTGAGAGAGTCCGGCCCTGCCCTGGTGAAACCCACCCAGACCCTGACCCT |
| | | GACATGCACCTTCTCCGGCTTCTCCCTGTCCAACTTCGGCGAGGGCCTATCCTGGA |
| | | TTCGGCAGCCTCCTGGCAAGGGCCTGGAATGGCTGGCCCACATCTACTGGGACGAC |
| | | GACAAGCGGTACAACCCCAGCCTGAAGTCCCGGCTGACCATCTCCAAGGACACCTC |
| | | CCGGAACCAGGTGGTGCTGACCATGACCAACATGGACCCCGTGGACACCGCCACCT |
| | | ACTACTGCGCCAGACGGGAAACCGTGTTCTACTGGTACTTCGACGTGTGGGGCCAG |
| | | GGCACCACCGTGACCGTGTCCTCTGCGTCGACCAAGGGCCCATCGGTCTTCCCCCT |
| | | GGCACCCTCCTCCAAGAGCACCTCTGGGGGCACAGCGGCCCTGGGCTGCCTGGTCA |
| | | AGGACTACTTCCCCGAACCGGTGACGGTGTCGTGGAACTCAGGCGCCCTGACCAGC |
| | | GGCGTGCACACCTTCCCGGCTGTCCTACAGTCCTCAGGACTCTACTCCCTCAGCAG |
| | | CGTGGTGACCGTGCCCTCCAGCAGCTTGGGCACCCAGACCTACATCTGCAACGTGA |
| | | ATCACAAGCCCAGCAACACCAAGGTGGACAAGAAAGTTGAGCCCAAATCTTGTGAG |
| | | AAAACTCACACATGCCCACCGTGCCCAGCACCTGAAGCCGCTGGGGCACCGTCAGT |
| | | CTTCCTCTTCCCCCCAAAACCCAAGGACACCCTCATGATCTCCCGGACCCCTGAGG |
| | | TCACATGCGTGGTGGTGGACGTGAGCCACGAAGACCCTGAGGTCAAGTTCAACTGG |
| | | TACGTGGACGGCGTGGAGGTGCATAATGCCAAGACAAAGCCGCGGGAAGAGCAGTA |
| | | CAACAGCACGTACCGTGTGGTCAGCGTCCTCACCGTCCTGCACCAGGACTGGCTGA |
| | | ATGGCAAGGAGTACAAGTGCAAGGTCTCCAACAAAGCCCTCCCAGCCCCCATCGAG |
| | | AAAACCATCTCCAAAGCCAAAGGGCAGCCCCGAGAACCACAGGTGTACACCCTGCC |
| | | CCCATCCCGGGAGGAGATGACCAAGAACCAGGTCAGCCTGACCTGCGAGGTCAAAG |
| | | GCTTCTATCCCAGCGACATCGCCGTGGAGTGGGAGAGCAATGGGCAGCCGGAGAAC |
| | | AACTACAAGACCACGCCTCCCGTGCTGGACTCCGACGGCTCCTTCTTCCTCTATAG |
| | | CAAGCTCACCGTGGACAAGAGCAGGTGGCAGCAGGGGAACGTCTTCTCATGCTCCG |
| | | TGCTGCATGAGGCTCTGCACAGCCACTACACGCAGAAGAGCCTCTCCCTGTCCCCG |
| | | GGAAAA |
| 189 | DFab IL4-LC (nucleic | GACATCCAGATGACCCAGTCTCCATCCTCCCTGTCTGCATCTGTGGGAGACAGAGT |
| | acid) | CACCATCACTTGCCGGGCCTCCCAGTCCGTGGACGAAGAGGGCGACTCCTACATGA |
| | | ACTGGTATCAGCAGAAACCAGGGAAAGCTCCTAAGCTCCTGATCTATGCCGCCTCC |
| | | AACCTGGAATCCGGCGTCCCATCAAGGTTCAGTGGCAGTGGATCTGGCACAGATTT |
| | | CACTCTCACCATCAGCAGTCTGCAACCTGAAGATTTTGCAACTTACTACTGTCAGC |
| | | AGTCCAACAAGGACCCCCCCACCTTCGGCGGAGGGACCAAGGTGGAGATCAAACGT |
| | | ACGGTGGCTGCACCATCTGTCTTCATCTTCCCGCCATCTGATGAGCAGTTGAAATC |
| | | TGGAACTGCCTCTGTTGTGTGCCTGCTGAATAACTTCTATCCCAGAGAGGCCAAAG |
| | | TACAGTGGAAGGTGGATAACGCCCTCCAATCGGGTAACTCCCAGGAGAGTGTCACA |
| | | GAGCAGGACAGCAAGGACAGCACCTACAGCCTCAGCAGCACCCTGACGCTGAGCAA |
| | | AGCAGACTACGAGAAACACAAAGTCTACGCCTGCGAAGTCACCCATCAGGGCCTGA |
| | | GCTCGCCCGTCACAAAGAGCTTCAACAGGGGAGAGTGT |
| 190 | SFab IL33-HC | GAGGTGCAGCTGGTGGAGTCTGGCGGCGGACTGGTGCAGCCTGGCGGCTCTCTGAG |
| | [IL13433-1258 SFab | ACTGTCTTGTGCCGCCTCCGGCTTCACCTTCAGTTCCTACTGGATGTACTGGGTGA |
| | HC] (nucleic acid) | GGCAGGCCCCTGGCAAGGGCCTGGAGTGGGTGGCCGCCATTACTCCTAATGCCGGT |
| | | GAGGACTACTATCCAGAGTCTGTGAAAGGCCGGTTCACCATCTCCAGGGACAACGC |
| | | CAAGAACTCCCTGTACCTCCAGATGAACTCCCTGAGGGCCGAGGATACCGCCGTGT |
| | | ACTACTGTGCCAGAGGCCAGTATTACTATACCAAGTATTCCCTTGGATACTGGGGC |
| | | CAGGGCACCCTGGTGACCGTGTCCTCTGCGTCGACCAAGGGCCCATCGGTCTTCCC |
| | | CCTGGCACCCTCCTCCAAGAGCACCTCTGGGGGCACAGCGGCCCTGGGCTGCCTGG |
| | | TCAAGGACTACTTCCCCGAACCGGTGACGGTGTCGTGGAACTCAGGCGCCCTGACC |
| | | AGCGGCGTGCACACCTTCCCGGCTGTCCTACAGTCCTCAGGACTCTACTCCCTCAG |
| | | CAGCGTGGTGACCGTGCCCTCCAGCAGCTTGGGCACCCAGACCTACATCTGCAACG |
| | | TGAATCACAAGCCCAGCAACACCAAGGTGGACAAGAAAGTTGAGCCCAAATCTTGT |
| | | CGGAAAACTCACACATGCCCACCGTGCCCAGCACCTGAAGCCGCTGGGGCACCGTC |
| | | AGTCTTCCTCTTCCCCCCAAAACCCAAGGACACCCTCATGATCTCCCGGACCCCTG |
| | | AGGTCACATGCGTGGTGGTGGACGTGAGCCACGAAGACCCTGAGGTCAAGTTCAAC |
| | | TGGTACGTGGACGGCGTGGAGGTGCATAATGCCAAGACAAAGCCGCGGGAGGAGCA |
| | | GTACAACAGCACGTACCGTGTGGTCAGCGTCCTCACCGTCCTGCACCAGGACTGGC |
| | | TGAATGGCAAGGAGTACAAGTGCAAGGTCTCCAACAAAGCCCTCCCAGCCCCCATC |
| | | GAGAAAACCATCTCCAAAGCCAAAGGGCAGCCCCGAGAACCACAGGTGTACACCCT |
| | | GCCCCCATCCCGGGAGGAGATGACCAAGAACCAGGTCAGCCTGACCTGCCTGGTCA |
| | | AAGGCTTCTATCCCAGCGACATCGCCGTGGAGTGGGAGAGCAATGGGCAGCCGGAG |
| | | AACAACTACAAGACCACGCCTCCCGTGCTGGACTCCGACGGCTCCTTCTTCCTCTA |
| | | TAGCAGGCTCACCGTGGACAAGAGCAGGTGGCAGCAGGGGAACGTCTTCTCATGCT |
| | | CCGTGCTGCATGAGGCTCTGCACAGCCACTACACGCAGAAGAGCCTCTCCCTGTCC |
| | | CCGGGAAAA |
| 191 | SFab IL33-LC | GACATCCAGATGACCCAGTCCCCCTCTTCTCTGTCTGCCTCTGTGGGCGACAGAGT |
| | [IL13433-1258 SFab | GACCATCACCTGTAGAGCAAGTCAGCCTATTCACAACCACTTGGACTGGTATCAGC |
| | HC] (nucleic acid) | AGAAGCCTGGCAAGGCTCCCAAGCTGCTGATCTACTTTGGAAAGAATTTGCAAGAA |
| | | GGCGTGCCTTCCAGATTCTCCGGCTCTGGCTCTGGCACCGATTTCACCCTGACCAT |
| | | CTCCTCCCTCCAGCCTGAGGATTTCGCCACCTACTACTGCTTTCAGTATAAGAAGG |
| | | GGTGGAGCTTTGGCGGCGGAACAAAGGTGGAGATCAAGCGTACGGTGGCTGCACCA |
| | | TCTGTCTTCATCTTCCCGCCATCTGATGAGCAGTTGAAATCTGGAACTGCCTCTGT |
| | | TGTGTGCCTGCTGAATAACTTCTATCCCAGAGAGGCCAAAGTACAGTGGAAGGTGG |
| | | ATAACGCCCTCCAATCGGGTAACTCCCAGGAGAGTGTCACAGAGCAGGACAGCAAG |
| | | GACAGCACCTACAGCCTCAGCAGCACCCTGACGCTGAGCAAAGCAGACTACGAGAA |
| | | ACACAAAGTCTACGCCTGCGAAGTCACCCATCAGGGCCTGAGCTCGCCCGTCACAA |
| | | AGAGCTTCAACAGGGGAGAGTGT |
| 192 | SFab TSLP-HC | CAGATGCAGCTGGTGGAGTCTGGGGGAGGCGTGGTCCAGCCTGGGAGGTCCCTGAG |
| | [IL413TSLP-1024, | ACTCTCCTGTGCAGCGTCTGGATTCACCTTCAGAACCTATGGCATGCACTGGGTCC |
| | IL413TSLP-1028, | GCCAGGCTCCAGGCAAGGGGCTGGAGTGGGTGGCAGTTATATGGTATGATGGAAGT |
| | IL413TSLP-1037 | AATAAACACTATGCAGACTCCGTGAAGGGCCGATTCACCATCTCCAGAGACAATTC |
| | anti-TSLP single Fab | CAAGAACACGCTGTATCTGCAAATGAACAGCCTGAGAGCCGAGGACACGGCTGTGT |
| | heavy chain] (nucleic | ATTACTGTGCGAGAGCCCCTCAGTGGTACCTCGTTCATGAGGCTTTTGATATCTGG |
| | acid) | GGCCAAGGGACAATGGTCACCGTCTCTTCAGCGTCGACCAAGGGCCCATCGGTCTT |
| | | CCCCCTGGCACCCTCCTCCAAGAGCACCTCTGGGGGCACAGCGGCCCTGGGCTGCC |
| | | TGGTCAAGGACTACTTCCCCGAACCGGTGACGGTGTCGTGGAACTCAGGCGCCCTG |
| | | ACCAGCGGCGTGCACACCTTCCCGGCTGTCCTACAGTCCTCAGGACTCTACTCCCT |
| | | CAGCAGCGTGGTGACCGTGCCCTCCAGCAGCTTGGGCACCCAGACCTACATCTGCA |
| | | ACGTGAATCACAAGCCCAGCAACACCAAGGTGGACAAGAAAGTTGAGCCCAAATCT |
| | | TGTCGGAAAACTCACACATGCCCACCGTGCCCAGCACCTGAAGCCGCTGGGGCACC |
| | | GTCAGTCTTCCTCTTCCCCCCAAAACCCAAGGACACCCTCATGATCTCCCGGACCC |
| | | CTGAGGTCACATGCGTGGTGGTGGACGTGAGCCACGAAGACCCTGAGGTCAAGTTC |
| | | AACTGGTACGTGGACGGCGTGGAGGTGCATAATGCCAAGACAAAGCCGCGGGAGGA |
| | | GCAGTACAACAGCACGTACCGTGTGGTCAGCGTCCTCACCGTCCTGCACCAGGACT |
| | | GGCTGAATGGCAAGGAGTACAAGTGCAAGGTCTCCAACAAAGCCCTCCCAGCCCCC |
| | | ATCGAGAAAACCATCTCCAAAGCCAAAGGGCAGCCCCGAGAACCACAGGTGTACAC |
| | | CCTGCCCCCATCCCGGGAGGAGATGACCAAGAACCAGGTCAGCCTGACCTGCCTGG |
| | | TCAAAGGCTTCTATCCCAGCGACATCGCCGTGGAGTGGGAGAGCAATGGGCAGCCG |
| | | GAGAACAACTACAAGACCACGCCTCCCGTGCTGGACTCCGACGGCTCCTTCTTCCT |
| | | CTATAGCAGGCTCACCGTGGACAAGAGCAGGTGGCAGCAGGGGAACGTCTTCTCAT |
| | | GCTCCGTGCTGCATGAGGCTCTGCACAGCCACTACACGCAGAAGAGCCTCTCCCTG |
| | | TCCCCGGGAAAA |
| 193 | SFab TSLP-LC | TCCTATGTGCTGACTCAGCCACCCTCCGTGTCAGTGGCCCCAGGACAGACAGCCAG |
| | [IL413TSLP-1024 anti- | GATTACCTGTGGGGGAAACAACCTTGGAAGTAAAAGTGTGCACTGGTACCAGCAGA |
| | TSLP single Fab light | AGCCAGGCCAGGCCCCTGTGCTGGTCGTCTATGATGATAAGGACCGGCCCTCATGG |
| | chain] (nucleic acid) | ATTCCTGAGCGATTCTCTGGCTCCAACTCTGGGAACACCGCCACCCTGACCATCAG |
| | | CAGGGGCGAAGCCGGGGATGAGGCCGACTATTACTGTCAGGTGTGGGATAGTAAGA |
| | | GTGATCATGTGGTATTCGGCGGAGGGACCAAGCTGACCGTCCTAGGTCAGCCCAAG |
| | | GCTGCCCCCTCGGTCACTCTGTTCCCGCCCTCCTCTGAGGAGCTTCAAGCCAACAA |
| | | GGCCACACTGGTGTGTCTCATAAGTGACTTCTACCCGGGAGCCGTGACAGTGGCCT |
| | | GGAAGGCAGATAGCAGCCCCGTCAAGGCGGGAGTGGAGACCACCACACCCTCCAAA |
| | | CAAAGCAACAACAAGTACGCGGCCAGCAGCTATCTGAGCCTGACGCCTGAGCAGTG |
| | | GAAGTCCCACAGAAGCTACAGCTGCCAGGTCACGCATGAAGGGAGCACCGTGGAGA |
| | | AGACAGTGGCCCCTACAGAATGTTCA |
| 194 | SFab p40-HC | GAGGTGCAGCTGGTGCAGTCTGGAGCAGAGGTGAAAAAGCCCGGGGAGTCTCTGAA |
| | [IL413p40-0705 SFab | GATCTCCTGTAAGGGTTCTGGATACAGCTTTACCACCTACTGGCTGGGCTGGGTGC |
| | HC (3] (nucleic acid) | GCCAGATGCCCGGGAAAGGCCTGGATTGGATAGGGATCATGAGCCCTGTGGACTCT |
| | | GATATTAGATACAGCCCCTCCTTCCAAGGCCAGGTCACCATGTCAGTGGACAAGTC |
| | | CATCACCACCGCCTACCTGCAGTGGAATAGCCTGAAGGCCTCCGACACCGCCATGT |
| | | ATTACTGTGCCAGACGACGCCCTGGTCAAGGTTATTTCGATTTTTGGGGGCAAGGA |
| | | ACATTGGTGACCGTCAGTTCAGCGTCGACCAAGGGCCCATCGGTCTTCCCCCTGGC |
| | | ACCCTCCTCCAAGAGCACCTCTGGGGGCACAGCGGCCCTGGGCTGCCTGGTCAAGG |
| | | ACTACTTCCCCGAACCGGTGACGGTGTCGTGGAACTCAGGCGCCCTGACCAGCGGC |
| | | GTGCACACCTTCCCGGCTGTCCTACAGTCCTCAGGACTCTACTCCCTCAGCAGCGT |
| | | GGTGACCGTGCCCTCCAGCAGCTTGGGCACCCAGACCTACATCTGCAACGTGAATC |
| | | ACAAGCCCAGCAACACCAAGGTGGACAAGAAAGTTGAGCCCAAATCTTGTCGGAAA |
| | | ACTCACACATGCCCACCGTGCCCAGCACCTGAAGCCGCTGGGGCACCGTCAGTCTT |
| | | CCTCTTCCCCCCAAAACCCAAGGACACCCTCATGATCTCCCGGACCCCTGAGGTCA |
| | | CATGCGTGGTGGTGGACGTGAGCCACGAAGACCCTGAGGTCAAGTTCAACTGGTAC |
| | | GTGGACGGCGTGGAGGTGCATAATGCCAAGACAAAGCCGCGGGAGGAGCAGTACAA |
| | | CAGCACGTACCGTGTGGTCAGCGTCCTCACCGTCCTGCACCAGGACTGGCTGAATG |
| | | GCAAGGAGTACAAGTGCAAGGTCTCCAACAAAGCCCTCCCAGCCCCCATCGAGAAA |
| | | ACCATCTCCAAAGCCAAAGGGCAGCCCCGAGAACCACAGGTGTACACCCTGCCCCC |
| | | ATCCCGGGAGGAGATGACCAAGAACCAGGTCAGCCTGACCTGCCTGGTCAAAGGCT |
| | | TCTATCCCAGCGACATCGCCGTGGAGTGGGAGAGCAATGGGCAGCCGGAGAACAAC |
| | | TACAAGACCACGCCTCCCGTGCTGGACTCCGACGGCTCCTTCTTCCTCTATAGCAG |
| | | GCTCACCGTGGACAAGAGCAGGTGGCAGCAGGGGAACGTCTTCTCATGCTCCGTGC |
| | | TGCATGAGGCTCTGCACAGCCACTACACGCAGAAGAGCCTCTCCCTGTCCCCGGGA |
| | | AAA |
| 195 | SFab p40-LC | GACATCCAGATGACCCAGTCTCCATCCTCACTGTCTGCATCTGTGGGAGACAGAGT |
| | [IL413p40-0705 LC | CACCATCACTTGTCGGGCCAGTCAGGGTATTAGCAGCTGGCTGGCCTGGTATCAGC |
| | (3)] (nucleic acid) | AGAAACCAGAGAAAGCCCCTAAGTCCCTGATCTATGCTGCATCCAGTTTGCAAAGT |
| | | GGGGTCCCATCAAGGTTCAGCGGCAGTGGATCTGGGACAGATTTCACTCTCACCAT |
| | | CAGCAGCCTGCAGCCTGAAGATTTTGCAACTTATTACTGCCAACAGTATAATATTT |
| | | ATCCCTACACCTTCGGACAAGGTACAAAACTGGAGATCAAACGAACTGTGGCTGCA |
| | | CCATCTGTCTTCATCTTCCCGCCATCTGATGAGCAGTTGAAATCTGGAACTGCCTC |
| | | TGTTGTGTGCCTGCTGAATAACTTCTATCCCAGAGAGGCCAAAGTACAGTGGAAGG |
| | | TGGATAACGCCCTCCAATCGGGTAACTCCCAGGAGAGTGTCACAGAGCAGGACAGC |
| | | AAGGACAGCACCTACAGCCTCAGCAGCACCCTGACGCTGAGCAAAGCAGACTACGA |
| | | GAAACACAAAGTCTACGCCTGCGAAGTCACCCATCAGGGCCTGAGCTCGCCCGTCA |
| | | CAAAGAGCTTCAACAGGGGAGAGTGT |
| 196 | IL13433-0006 LC (3) | DIQMTQSPSSLSASVGDRVTITCKASESVDHFGWSLVHWYQQKPGKAPKLLIYRAS |
| | | NLESGVPSRFSGSGSGTDFTLTISSLQPEDFATYYCQQSNEDPWTFGGGTKVEIKR |
| | | TVAAPSVFIFPPSDEQLKSGTASVSCLLNNFYPREAKVQWKVDNALQSGNSQESVT |
| | | EQDSKDSTYSLDSTLTLSKADYEKHKVYACEVTHQGLSSPVTKSENRGEC |
| 197 | IL4-1305 LC | DIQMTQSPSSLSASVGDRVTITCKASQSVDEDGDSYMNWYQQKPGKAPKLLIYAAS |
| | [IL13433-1305 LC] | NLESGVPSRFSGSGSGTDFTLTISSLQPEDFATYYCQQSNEDPPTFGGGTKVEIKR |
| | (hu3B9_G07_VLv2.9 LC) | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGEC |
| 198 | IL13-0001 VH | GAGGTGCAGCTGGTGGAGTCAGGGGGAGGCTTGGTGCAACCTGGAGGGTCCCTGAG |
| | | ACTCTCCTGTGCAGCCTCTGGATTCACTTTCAGTAGCTATGCCATGTCTTGGGTTC |
| | | GTCAGGCTCCAGGGAAGGGGCTGGAGTGGGTCGCATCCATTAGTAGTGGTGACACC |
| | | ACCTACTATCCAGACAGCGTGAAGGGCCGATTCACCATCTCCAGAGATAATGCCAA |
| | | GAACAGCCTGTACCTGCAAATGAACAGTCTGAGGGCTGAGGACACCGCCGTGTATT |
| | | ACTGTGCACGAAACGAGGGTTACTACTTCGGACTGACCCTGTGGGGCCAAGGGACC |
| | | CTGGTCACCGTCTCCTCT |
| 199 | IL13-0001 VL | GACATCCAGATGACCCAGTCTCCATCCTCCCTGTCTGCATCTGTGGGAGACAGAGT |
| | | CACCATCACTTGCAAAGCCAGTGAAAGTGTTGATCACTTTGGTTGGTCTCTCGTGC |
| | | ACTGGTATCAGCAGAAACCAGGGAAAGCTCCTAAGCTCCTGATCTATCGTGCATCC |
| | | AACCTGGAATCTGGCGTCCCATCAAGGTTCAGTGGCAGTGGATCTGGCACAGATTT |
| | | CACTCTCACCATCAGCAGTCTGCAACCTGAAGATTTTGCAACTTACTACTGTCAGC |
| | | AAAGTAATGAAGATCCCTGGACCTTCGGCGGAGGGACCAAGGTGGAGATCAAA |
| 200 | IL4-1040 VH | GAGGTCACACTGAGAGAGTCCGGCCCTGCCCTGGTGAAACCCACCCAGACCCTGAC |
| | | CCTGACATGCACCTTCTCCGGCTTCTCCCTGTCCAACTTCGGCGAGGGCCTATCCT |
| | | GGATTCGGCAGCCTCCTGGCAAGGGCCTGGAATGGCTGGCCCACATCTACTGGGAC |
| | | GACGACAAGCGGTACAACCCCAGCCTGAAGTCCCGGCTGACCATCTCCAAGGACAC |
| | | CTCCCGGAACCAGGTGGTGCTGACCATGACCAACATGGACCCCGTGGACACCGCCA |
| | | CCTACTACTGCGCCAGACGGGAAACCGTGTTCTACTGGTACTTCGACGTGTGGGGC |
| | | CAGGGCACCACCGTGACCGTGTCCTCT |
| 201 | IL4-1040 VL | GACATCCAGATGACCCAGTCTCCATCCTCCCTGTCTGCATCTGTGGGAGACAGAGT |
| | | CACCATCACTTGCCGGGCCTCCCAGTCCGTGGACGAAGAGGGCGACTCCTACATGA |
| | | ACTGGTATCAGCAGAAACCAGGGAAAGCTCCTAAGCTCCTGATCTATGCCGCCTCC |
| | | AACCTGGAATCCGGCGTCCCATCAAGGTTCAGTGGCAGTGGATCTGGCACAGATTT |
| | | CACTCTCACCATCAGCAGTCTGCAACCTGAAGATTTTGCAACTTACTACTGTCAGC |
| | | AGTCCAACAAGGACCCCCCCACCTTCGGCGGAGGGACCAAGGTGGAGATCAAA |
| 202 | IL33-0726 VH | GAGGTGCAGCTGGTGGAGTCTGGCGGCGGACTGGTGCAGCCTGGCGGCTCTCTGAG |
| | | ACTGTCTTGTGCCGCCTCCGGCTTCACCTTCAGTTCCTACTGGATGTACTGGGTGA |
| | | GGCAGGCCCCTGGCAAGGGCCTGGAGTGGGTGGCCGCCATTACTCCTAATGCCGGT |
| | | GAGGACTACTATCCAGAGTCTGTGAAAGGCCGGTTCACCATCTCCAGGGACAACGC |
| | | CAAGAACTCCCTGTACCTCCAGATGAACTCCCTGAGGGCCGAGGATACCGCCGTGT |
| | | ACTACTGTGCCAGAGGCCAGTATTACTATACCAAGTATTCCCTTGGATACTGGGGC |
| | | CAGGGCACCCTGGTGACCGTGTCCTCT |
| 203 | IL33-0726 VL | GACATCCAGATGACCCAGTCCCCCTCTTCTCTGTCTGCCTCTGTGGGCGACAGAGT |
| | | GACCATCACCTGTAGAGCAAGTCAGCCTATTCACAACCACTTGGACTGGTATCAGC |
| | | AGAAGCCTGGCAAGGCTCCCAAGCTGCTGATCTACTTTGGAAAGAATTTGCAAGAA |
| | | GGCGTGCCTTCCAGATTCTCCGGCTCTGGCTCTGGCACCGATTTCACCCTGACCAT |
| | | CTCCTCCCTCCAGCCTGAGGATTTCGCCACCTACTACTGCTTTCAGTATAAGAAGG |
| | | GGTGGAGCTTTGGCGGCGGAACAAAGGTGGAGATCAAG |
| 204 | TSLP-0875 VH | CAGATGCAGCTGGTGGAGTCTGGGGGAGGCGTGGTCCAGCCTGGGAGGTCCCTGAG |
| | | ACTCTCCTGTGCAGCGTCTGGATTCACCTTCAGAACCTATGGCATGCACTGGGTCC |
| | | GCCAGGCTCCAGGCAAGGGGCTGGAGTGGGTGGCAGTTATATGGTATGATGGAAGT |
| | | AATAAACACTATGCAGACTCCGTGAAGGGCCGATTCACCATCTCCAGAGACAATTC |
| | | CAAGAACACGCTGTATCTGCAAATGAACAGCCTGAGAGCCGAGGACACGGCTGTGT |
| | | ATTACTGTGCGAGAGCCCCTCAGTGGTACCTCGTTCATGAGGCTTTTGATATCTGG |
| | | GGCCAAGGGACAATGGTCACCGTCTCTTCA |
| 205 | TSLP-0875 VL | TCCTATGTGCTGACTCAGCCACCCTCCGTGTCAGTGGCCCCAGGACAGACAGCCAG |
| | TSLP-2002 VL | GATTACCTGTGGGGGAAACAACCTTGGAAGTAAAAGTGTGCACTGGTACCAGCAGA |
| | | AGCCAGGCCAGGCCCCTGTGCTGGTCGTCTATGATGATAAGGACCGGCCCTCATGG |
| | | ATTCCTGAGCGATTCTCTGGCTCCAACTCTGGGAACACCGCCACCCTGACCATCAG |
| | | CAGGGGCGAAGCCGGGGATGAGGCCGACTATTACTGTCAGGTGTGGGATAGTAAGA |
| | | GTGATCATGTGGTATTCGGCGGAGGGACCAAGCTGACCGTCCTA |
| 206 | p40-0003 VH | GAGGTGCAGCTGGTGCAGTCTGGAGCAGAGGTGAAAAAGCCCGGGGAGTCTCTGAA |
| | | GATCTCCTGTAAGGGTTCTGGATACAGCTTTACCACCTACTGGCTGGGCTGGGTGC |
| | | GCCAGATGCCCGGGAAAGGCCTGGATTGGATAGGGATCATGAGCCCTGTGGACTCT |
| | | GATATTAGATACAGCCCCTCCTTCCAAGGCCAGGTCACCATGTCAGTGGACAAGTC |
| | | CATCACCACCGCCTACCTGCAGTGGAATAGCCTGAAGGCCTCCGACACCGCCATGT |
| | | ATTACTGTGCCAGACGACGCCCTGGTCAAGGTTATTTCGATTTTTGGGGGCAAGGA |
| | | ACATTGGTGACCGTCAGTTCA |
| 207 | p40-0003 VL | GACATCCAGATGACCCAGTCTCCATCCTCACTGTCTGCATCTGTGGGAGACAGAGT |
| | | CACCATCACTTGTCGGGCCAGTCAGGGTATTAGCAGCTGGCTGGCCTGGTATCAGC |
| | | AGAAACCAGAGAAAGCCCCTAAGTCCCTGATCTATGCTGCATCCAGTTTGCAAAGT |
| | | GGGGTCCCATCAAGGTTCAGCGGCAGTGGATCTGGGACAGATTTCACTCTCACCAT |
| | | CAGCAGCCTGCAGCCTGAAGATTTTGCAACTTATTACTGCCAACAGTATAATATTT |
| | | ATCCCTACACCTTCGGACAAGGTACAAAACTGGAGATCAAA |
| 208 | IL4-0749 LC | DIQMTQSPSSLSASVGDRVTITCRASQSVDEEGDSYMNWYQQKPGKAPKLLIYAAS |
| | [IL13433-0749 LC] | NLESGVPSRFSGSGSGTDFTLTISSLQPEDFATYYCQQSNEDPPTFGGGTKVEIKR |
| | | TVAAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSENRGEC |
| 209 | IL13433-0717 HC (3) | EVQLVESGGGLVQPGGSLRLSCAASGFTFSSYAMSWVRQAPGKGLEWVASISSGDT |
| | | TYYPDSVKGRFTISRDNAKNSLYLQMNSLRAEDTAVYYCARNEGYYFGLTLWGQGT |
| | | LVTVSSASTKGPSVFPKAPSSKSTSGGTAALGCLVKDYFPEPVTVSWNSGALTSGV |
| | | HTFPAVLQSSGLYSLSSSVTVPSSSLGTQTYICNVNHKPSNTKVDKKVEPKSCDKT |
| | | HTCPPCPAPEAAGAPSVFLFPPKPKDTLMISRTPEVTCVVVDVSHEDPEVKENWYV |
| | | DGVEVHNAKTKPREEQYNSTYRVVSVLTVLHQDWLNGKEYKCKVSNKALPAPIEKT |
| | | ISKAKGQPREPQVCTLPPSREEMTKNQVSLWCLVKGFYPSDIAVEWESNGQPENNY |
| | | KTTPPVLDSDGSFFLYSKLTVDKSRWQQGNVFSCSVLHEALHSHYTQKSLSLSPGK |
| 210 | TSLP-0100 VH | QMQLVESGGGVVQPGRSLRLSCAASGFTFRTYGMHWVRQAPGKGLEWVAVIWYDGS |
| | | NKHYADSVKGRFTISRDNSKNTLYLQMNSLRAEDTAVYYCARAPQWELVHEAFDIW |
| | | GQGTMVTVSS |
| 211 | | QVWDSSSKHVV |
| 212 | TSLP-0871 CDRL3 | QVWDKSSDHVV |
| 213 | TSLP-0855 VL | SYVLTQPPSVSVAPGQTARITCGGNNLGSKSVHWYQQKPGQAPVLVVYDDSDRPSW |
| | TSLP-2000 VL | IPERFSGSNSGNTATLTISRGEAGDEADYYCQVWDSSSKHVVFGGGTKLTVL |
| | IL413TSLP-1028 VL | |
| 214 | TSLP-0871 VL | SYVLTQPPSVSVAPGQTARITCGGNNLGSKSVHWYQQKPGQAPVLVVYDDKDRPSW |
| | IL413TSLP-1037 VL | IPERFSGSNSGNTATLTISRGEAGDEADYYCQVWDKSSDHVVFGGGTKLTVL |
| 215 | TSLP-0855 LC | SYVLTQPPSVSVAPGQTARITCGGNNLGSKSVHWYQQKPGQAPVLVVYDDSDRPSW |
| | TSLP-2000 LC | IPERFSGSNSGNTATLTISRGEAGDEADYYCQVWDSSSKHVVFGGGTKLTVLGQPK |
| | IL413TSLP-1028 LC | AAPSVTLFPPSSEELQANKATLVCLISDFYPGAVTVAWKADSSPVKAGVETTTPSK |
| | | QSNNKYAASSYLSLTPEQWKSHRSYSCQVTHEGSTVEKTVAPTECS |
| 216 | TSLP-0871 LC | SYVLTQPPSVSVAPGQTARITCGGNNLGSKSVHWYQQKPGQAPVLVVYDDKDRPSW |
| | IL413TSLP-1037 LC | IPERFSGSNSGNTATLTISRGEAGDEADYYCQVWDKSSDHVVFGGGTKLTVLGQPK |
| | | AAPSVTLFPPSSEELQANKATLVCLISDFYPGAVTVAWKADSSPVKAGVETTTPSK |
| | | QSNNKYAASSYLSLTPEQWKSHRSYSCQVTHEGSTVEKTVAPTECS |
| 217 | TSLP-0855 VL | TCCTATGTGCTGACTCAGCCACCCTCCGTGTCAGTGGCCCCAGGACAGACAGCCA |
| | TSLP-2000 VL | GGATTACCTGTGGGGGAAACAACCTTGGAAGTAAAAGTGTGCACTGGTACCAGCA |
| | IL413TSLP-1028 VL | GAAGCCAGGCCAGGCCCCTGTGCTGGTCGTCTATGATGATAGCGACCGGCCCTCA |
| | | TGGATTCCTGAGCGATTCTCTGGCTCCAACTCTGGGAACACCGCCACCCTGACCA |
| | | TCAGCAGGGGCGAAGCCGGGGATGAGGCCGACTATTACTGTCAGGTGTGGGATAG |
| | | TAGTAGTAAGCATGTGGTATTCGGCGGAGGGACCAAGCTGACCGTCCTA |
| 218 | TSLP-0871 VL | TCCTATGTGCTGACTCAGCCACCCTCCGTGTCAGTGGCCCCAGGACAGACAGCCA |
| | IL413TSLP-1037 VL | GGATTACCTGTGGGGGAAACAACCTTGGAAGTAAAAGTGTGCACTGGTACCAGCA |
| | | GAAGCCAGGCCAGGCCCCTGTGCTGGTCGTCTATGATGATAAGGACCGGCCCTCA |
| | | TGGATTCCTGAGCGATTCTCTGGCTCCAACTCTGGGAACACCGCCACCCTGACCA |
| | | TCAGCAGGGGCGAAGCCGGGGATGAGGCCGACTATTACTGTCAGGTGTGGGATAA |
| | | GAGTAGTGATCATGTGGTATTCGGCGGAGGGACCAAGCTGACCGTCCTA |
| 219 | TSLP-0855 LC | TCCTATGTGCTGACTCAGCCACCCTCCGTGTCAGTGGCCCCAGGACAGACAGCCA |
| | TSLP-2000 LC | GGATTACCTGTGGGGGAAACAACCTTGGAAGTAAAAGTGTGCACTGGTACCAGCA |
| | IL413TSLP-1028 LC | GAAGCCAGGCCAGGCCCCTGTGCTGGTCGTCTATGATGATAGCGACCGGCCCTCA |
| | | TGGATTCCTGAGCGATTCTCTGGCTCCAACTCTGGGAACACCGCCACCCTGACCA |
| | | TCAGCAGGGGCGAAGCCGGGGATGAGGCCGACTATTACTGTCAGGTGTGGGATAG |
| | | TAGTAGTAAGCATGTGGTATTCGGCGGAGGGACCAAGCTGACCGTCCTAGGTCAG |
| | | CCCAAGGCTGCCCCCTCGGTCACTCTGTTCCCGCCCTCCTCTGAGGAGCTTCAAG |
| | | CCAACAAGGCCACACTGGTGTGTCTCATAAGTGACTTCTACCCGGGAGCCGTGAC |
| | | AGTGGCCTGGAAGGCAGATAGCAGCCCCGTCAAGGCGGGAGTGGAGACCACCACA |
| | | CCCTCCAAACAAAGCAACAACAAGTACGCGGCCAGCAGCTATCTGAGCCTGACGC |
| | | CTGAGCAGTGGAAGTCCCACAGAAGCTACAGCTGCCAGGTCACGCATGAAGGGAG |
| | | CACCGTGGAGAAGACAGTGGCCCCTACAGAATGTTCA |
| 220 | TSLP-0871 LC | TCCTATGTGCTGACTCAGCCACCCTCCGTGTCAGTGGCCCCAGGACAGACAGCCA |
| | IL413TSLP-1037 LC | GGATTACCTGTGGGGGAAACAACCTTGGAAGTAAAAGTGTGCACTGGTACCAGCA |
| | | GAAGCCAGGCCAGGCCCCTGTGCTGGTCGTCTATGATGATAAGGACCGGCCCTCA |
| | | TGGATTCCTGAGCGATTCTCTGGCTCCAACTCTGGGAACACCGCCACCCTGACCA |
| | | TCAGCAGGGGCGAAGCCGGGGATGAGGCCGACTATTACTGTCAGGTGTGGGATAA |
| | | GAGTAGTGATCATGTGGTATTCGGCGGAGGGACCAAGCTGACCGTCCTAGGTCAG |
| | | CCCAAGGCTGCCCCCTCGGTCACTCTGTTCCCGCCCTCCTCTGAGGAGCTTCAAG |
| | | CCAACAAGGCCACACTGGTGTGTCTCATAAGTGACTTCTACCCGGGAGCCGTGAC |
| | | AGTGGCCTGGAAGGCAGATAGCAGCCCCGTCAAGGCGGGAGTGGAGACCACCACA |
| | | CCCTCCAAACAAAGCAACAACAAGTACGCGGCCAGCAGCTATCTGAGCCTGACGC |
| | | CTGAGCAGTGGAAGTCCCACAGAAGCTACAGCTGCCAGGTCACGCATGAAGGGAG |
| | | CACCGTGGAGAAGACAGTGGCCCCTACAGAATGTTCA |
| 221 | TSLP-0820 VH | EMQLVESGGGLVQPGGSLRLSCAASGFTFRTYGMHWVRQAPGKGLEWVAVIWYDG |
| | TSLP-2000 VH | SNKHYADSVKGRFTISRDNAKNSLYLQMNSLRAEDTAVYYCARAPQWYLVHEAFD |
| | TSLP-2002 VH | IWGQGTMVTVSS |
| | TSLP-2004 VH | |
| 222 | TSLP-0820 HC | EMQLVESGGGLVQPGGSLRLSCAASGFTFRTYGMHWVRQAPGKGLEWVAVIWYDG |
| | TSLP-2000 HC | SNKHYADSVKGRFTISRDNAKNSLYLQMNSLRAEDTAVYYCARAPQWYLVHEAFD |
| | TSLP-2002 HC | IWGQGTMVTVSSASTKGPSVFPLAPSSKSTSGGTAALGCLVKDYFPEPVTVSWNS |
| | TSLP-2004 HC | GALTSGVHTFPAVLQSSGLYSLSSVVTVPSSSLGTQTYICNVNHKPSNTKVDKKV |
| | | EPKSCDKTHTCPPCPAPEAAGAPSVFLFPPKPKDTLMISRTPEVTCVVVDVSHED |
| | | PEVKFNWYVDGVEVHNAKTKPREEQYNSTYRVVSVLTVLHQDWLNGKEYKCKVSN |
| | | KALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHNH |
| | | YTQKSLSLSPG |
| 223 | TSLP-2004 VL | SYVLTQPPSVSVAPGQTARITCGGNNLGSKSVHWYQQKPGQAPVLVVYDDSDRPS |
| | | WIPERFSGSNSGNTATLTISRGEAGDEADYYCQVWDKSSDHVVFGGGTKLTVL |
| 224 | TSLP-2004 LC | SYVLTQPPSVSVAPGQTARITCGGNNLGSKSVHWYQQKPGQAPVLVVYDDSDRPS |
| | | WIPERFSGSNSGNTATLTISRGEAGDEADYYCQVWDKSSDHVVFGGGTKLTVLGQ |
| | | PKAAPSVTLFPPSSEELQANKATLVCLISDFYPGAVTVAWKADSSPVKAGVETTT |
| | | PSKQSNNKYAASSYLSLTPEQWKSHRSYSCQVTHEGSTVEKTVAPTECS |
| 225 | sasanlimab, (PF- | QVQLVQSGAEVKKPGASVKVSCKASGYTFTSYWINWVRQAPGQGLEWMGNIYPGSS |
| | 06801591 (RN888) | LTNYNEKFKNRVTMTRDTSTSTVYMELSSLRSEDTAVYYCARLSTGTFAYWGQGTL |
| | Anti-PD-1 | VTVSSASTKGPSVFPLAPCSRSTSESTAALGCLVKDYFPEPVTVSWNSGALTSGVH |
| | HC CDRs in bold and | TFPAVLQSSGLYSLSSVVTVPSSSLGTKTYTCNVDHKPSNTKVDKRVESKYGPPCP |
| | underlined. | PCPAPEFLGGPSVFLFPPKPKDTLMISRTPEVTCVVVDVSQEDPEVQFNWYVDGVE |
| | | VHNAKTKPREEQFNSTYRVVSVLTVLHQDWLNGKEYKCKVSNKGLPSSIEKTISKA |
| | | KGQPREPQVYTLPPSQEEMTKNQVSLTCLVKGFYPSDIAVEWESNGQPENNYKTTP |
| | | PVLDSDGSFFLYSRLTVDKSRWQEGNVFSCSVMHEALHNHYTQKSLSLSLGK |
| 226 | sasanlimab, (PF- | DIVMTQSPDSLAVSLGERATINCKSSQSLWDSGNQKNFLTWYQQKPGQPPKLLIYW |
| | 06801591 (RN888) | TSYRESGVPDRFSGSGSGTDFTLTISSLQAEDVAVYYCQNDYFYPHTFGGGTKVEI |
| | Anti-PD-1 | KRGTVAAPSVFIFPPSDEQLKSGTASVVCLLNNFYPREAKVQWKVDNALQSGNSQE |
| | LC | SVTEQDSKDSTYSLSSTLTLSKADYEKHKVYACEVTHQGLSSPVTKSENRGEC |
| | CDRs in bold and | |
| | underlined. | |
| 227 | PD1-F2 HC | EVQLVQSGGGVVQPGRSLRLSCAASGFTFSSYWMSWVRQAPGKGLEWVSAISGSGG |
| | | STYYADSVKGRFTISRDNSKNTLYLQMNSLRAEDTAVYYCAKENWGSYFDLWGQGT |
| | | TVTVSSAKTTPPSVYPLAPGSAAQTNSMVTLGCLVKGYFPEPVTVTWNSGSLSSGV |
| | | HTFPAVLQSDLYTLSSSVTVPSSTWPSETVTCNVAHPASSTKVDKKIVPRDCGCKP |
| | | CICTVPEVSSVFIFPPKPKDVLTITLTPKVTCVVVDISKDDPEVQFSWFVDDVEVH |
| | | TAQTQPREEQFNSTFRSVSELPIMHQDWLNGKEFKCRVNSAAFPAPIEKTISKTKG |
| | | RPKAPQVYTIPPPKEQMAKDKVSLTCMITDFFPEDITVEWQWNGQPAENYKNTQPI |
| | | MDTDGSYFIYSKLNVQKSNWEAGNTFTCSVLHEGLHNHHTEKSLSHSPGK |
| 228 | PD1-F2 LC | DIVMTQSPSTLSASVGDRVTITCRASQGISSWLAWYQQKPGRAPKVLIYKASTLES |
| | | GVPSRFSGSGSGTDFTLTISSLQPEDFATYYCQQSYSTPWTFGQGTKLEIKRADAA |
| | | PTVSIFPPSSEQLTSGGASVVCFLNNFYPKDINVKWKIDGSERQNGVLNSWTDQDS |
| | | KDSTYSMSSTLTLTKDEYERHNSYTCEATHKTSTSPIVKSENRNEC |

[ ] = Genedata alias; ( ) = Legacy alias.

1. An isolated antibody that specifically binds to IL-4, comprising a heavy chain variable region (IL4-VH) and a light chain variable region (IL4-VL), comprising
(i) a CDR-H1, CDR-H2, and CDR-H3 sequences of SEQ ID NO: 22, and a CDR-L1, CDR-L2, and CDR-L3 sequences of SEQ ID NO: 26
(ii) a CDR-H1, CDR-H2, and CDR-H3 sequences of SEQ ID NO: 19, and a CDR-L1, CDR-L2, and CDR-L3 sequences of SEQ ID NO: 20;
(iii) a CDR-H1, CDR-H2, and CDR-H3 sequences of SEQ ID NO: 22, and a CDR-L1, CDR-L2, and CDR-L3 sequences of SEQ ID NO: 20;
(iv) a CDR-H1, CDR-H2, and CDR-H3 sequences of SEQ ID NO: 28, and a CDR-L1, CDR-L2, and CDR-L3 sequences of SEQ ID NO: 29; or
(v) a CDR-H1, CDR-H2, and CDR-H3 sequences of SEQ ID NO: 22, and a CDR-L1, CDR-L2, and CDR-L3 sequences of SEQ ID NO: 30.

2. The antibody of claim 1, wherein the IL-4 binding domain comprises
(i) an IL4-VH sequence of SEQ ID NO: 22, and an IL4-VL of SEQ ID NO: 26;
(ii) an IL4-VH sequence of SEQ ID NO: 19, and an IL4-VL of SEQ ID NO: 20;
(iii) an IL4-VH sequence of SEQ ID NO: 22, and an IL4-VL of SEQ ID NO: 20;
(iv) an IL4-VH sequence of SEQ ID NO: 28, and an IL4-VL of SEQ ID NO: 29;
(v) an IL4-VH sequence of SEQ ID NO: 22, and an IL4-VL of SEQ ID NO: 30;
(vi) an IL4-VH sequence encoded by a nucleic acid sequence of SEQ ID NO: 200, and an IL4-VL sequence encoded by a nucleic acid sequence of SEQ ID NO: 201;
(vii) an IL4-VH bearing polypeptide sequence encoded by a nucleic acid sequence of SEQ ID NO: 188, and an IL4-VL bearing polypeptide sequence encoded by a nucleic acid sequence of SEQ ID NO: 189;
(viii) an IL4-VH sequence encoded by the plasmid deposited at the ATCC and having ATCC Accession No. PTA-127198, and an IL4-VL sequence encoded by the plasmid deposited at the ATCC and having ATCC Accession No. PTA-127197;
(ix) an IL4-VH bearing polypeptide sequence encoded by the plasmid deposited at the ATCC and having ATCC Accession No. PTA-127192, and an IL4-VL bearing polypeptide sequence encoded by the plasmid deposited at the ATCC and having ATCC Accession No. PTA-127194.

3. The antibody of claim 1, characterized by one or more of the following:
(i) the antibody binds human IL-4 with a K_{D} less than a value selected from the group consisting of about, 10 pM, 5 pM, 1 pM, and 800 fM;
(ii) the antibody binds cynomolgus IL-4;
(iii) the antibody does not bind IL-4 from one or more selected from the groups consisting of dog, sheep, rabbit, rat, and mouse;
(iv) the binding K_{D} of the antibody to cynomolgus IL-4 is within 1 order of magnitude of the binding K_{D} of the antibody to human IL-4;
(v) the antibody is characterized by an IC₅₀ of less than 10 pM in a human monocyte assay for neutralization of IL-4 induction of CD23;
(vi) the antibody has a viscosity of 20 cP or less at 25° C. at a concentration of 80 mg/mL in a Histidine-sucrose pH 5.8 buffer;
(vii) the antibody comprises a lysine at residue 93 in the light chain.

4. An isolated antibody that specifically binds to IL-13, comprising a heavy chain variable region (IL13-VH) and a light chain variable region (IL13-VL), comprising
(i) the CDR-H1, CDR-H2, and CDR-H3 sequences of SEQ ID NO: 51, and the CDR-11, CDR-L2, and CDR-L3 sequences of SEQ ID NO: 54
(ii) the CDR-H1, CDR-H2, and CDR-H3 sequences of SEQ ID NO: 44, and the CDR-11, CDR-L2, and CDR-L3 sequences of SEQ ID NO: 46;
(iii) the CDR-H1, CDR-H2, and CDR-H3 sequences of SEQ ID NO: 48, and the CDR-11, CDR-L2, and CDR-L3 sequences of SEQ ID NO: 49;
(iv) the CDR-H1, CDR-H2, and CDR-H3 sequences of SEQ ID NO: 48, and the CDR-11, CDR-L2, and CDR-L3 sequences of SEQ ID NO: 68; or
(v) the CDR-H1, CDR-H2, and CDR-H3 sequences of SEQ ID NO: 57, and the CDR-1, CDR-L2, and CDR-L3 sequences of SEQ ID NO: 59.

5. The antibody of claim 4, comprising
(i) an IL13-VH of SEQ ID NO: 51, and an IL13-VL of SEQ ID NO: 54
(ii) an IL13-VH of SEQ ID NO: 44, and an IL13-VL of SEQ ID NO: 46;
(iii) an IL13-VH of SEQ ID NO: 48, and an IL13-VL of SEQ ID NO: 49;
(iv) an IL13-VH of SEQ ID NO: 48, and an IL13-VL of SEQ ID NO: 68;
(v) an IL13-VH of SEQ ID NO: 57, and an IL13-VL of SEQ ID NO: 59;
(vi) an IL13-VH sequence encoded a nucleic acid sequence of SEQ ID NO: 198, and an IL13-VL sequence encoded a nucleic acid sequence of SEQ ID NO: 199;
(vii) an IL13-VH bearing polypeptide sequence encoded a nucleic acid sequence of SEQ ID NO: 187, and an IL13-VL bearing polypeptide sequence encoded a nucleic acid sequence of SEQ ID NO: 188;
(viii) an IL13-VH sequence encoded by the plasmid deposited at the ATCC and having ATCC Accession No. PTA-127196, and an IL13-VL sequence encoded by the plasmid deposited at the ATCC and having ATCC Accession No. PTA-127195; or
(ix) an IL13-VH bearing polypeptide sequence encoded by the plasmid deposited at the ATCC and having ATCC Accession No. PTA-127193, and an IL13-VL bearing polypeptide sequence encoded by the plasmid deposited at the ATCC and having ATCC Accession No. PTA-127192.

6. The antibody of claim 4, characterized by one or more of the following:
(i) the antibody binds human IL-13 with a K_{D} less than a value selected from the group consisting of 10 nM, 5 nM, 2 nM, 1 nM, 900 pM, 800 pM, 700 pM, 600 pM, 500 pM, 400 pM, 300 pM, 250 pM, 200 pM, 150 pM, 100 pM, and 60 pM;
(ii) the antibody binds cynomolgus IL-13, optionally within 1 order of magnitude of the binding K_{D} of the antibody to human IL-13;
(iii) the IL-13 IC₅₀ is less than 100 pM as measured by neutralization of IL-13 pSTAT6 phosphorylation in HT-29 cells;
(iv) the IL-13 IC₅₀ is less than 20 pM as measured in a human monocyte assay for neutralization of IL-13 induction of CD23;
(v) the antibody has a terminal half-life of at least 14 days in cynomolgus monkeys;
(vi) the antibody has a terminal half-life of at least 18 days in TG32 mice;
(vii) the antibody does not bind IL-13 from one or more species selected from the group consisting of dog, rabbit, and mouse.

7. The antibody of claim 1, that specifically binds to IL-4, and specifically binds to IL-13, comprising an IL-4 binding domain and an IL-13 binding domain, wherein
(i) the IL-4 binding domain comprises the antibody of claim 1, and
(ii) the IL-13 binding domain comprises a heavy chain variable region (IL13-VH) and a light chain variable region (IL13-VL), comprising
(i) the CDR-H1, CDR-H2, and CDR-H3 sequences of SEQ ID NO: 51, and the CDR-11, CDR-L2, and CDR-L3 sequences of SEQ ID NO: 54
(ii) the CDR-H1, CDR-H2, and CDR-H3 sequences of SEQ ID NO: 44, and the CDR-11, CDR-L2, and CDR-L3 sequences of SEQ ID NO: 46;
(iii) the CDR-H1, CDR-H2, and CDR-H3 sequences of SEQ ID NO: 48, and the CDR-11, CDR-L2, and CDR-L3 sequences of SEQ ID NO: 49;
(iv) the CDR-H1, CDR-H2, and CDR-H3 sequences of SEQ ID NO: 48, and the CDR-11, CDR-L2, and CDR-L3 sequences of SEQ ID NO: 68; or
(v) the CDR-H1, CDR-H2, and CDR-H3 sequences of SEQ ID NO: 57, and the CDR-11, CDR-L2, and CDR-L3 sequences of SEQ ID NO: 59.

8. The antibody of claim 7, wherein the antibody comprises a first, second, third, fourth, and fifth polypeptide chain, such that
a) the second and fifth polypeptide chains together form a first Fab domain comprising a first antigen binding site;
b) the second and fourth polypeptide chains together form a second Fab domain comprising a second antigen binding site;
c) the first and third polypeptide chains together form a third Fab domain comprising a third antigen binding site; and
wherein the first and second polypeptide chains associate together to form an antibody comprising two arms; a dual Fab arm comprising the first Fab domain and the second Fab domain, and a single Fab arm comprising the third Fab domain, and optionally wherein
(i) the first antigen binding site specifically binds IL-13, the second antibody binding site specifically binds IL-4, and the third antigen binding site specifically binds the at least one additional target;
(ii) first antigen binding site specifically binds IL-4, the second antibody binding site specifically binds IL-13, and the third antigen binding site specifically binds to the at least one additional target;
(iii) the first antigen binding site specifically binds IL-4, the second antibody binding site specifically binds the at least one additional target, and the third antigen binding site specifically binds IL-13;
(iv) wherein the first antigen binding site specifically binds IL-13, the second antibody binding site specifically binds the at least one additional target, and the third antigen binding site specifically binds IL-4;
(v) the first antigen binding site specifically binds the at least one additional target, the second antibody binding site specifically binds IL-13, and the third antigen binding site specifically binds IL-4; or
(vi) the first antigen binding site specifically binds the at least one additional target, the second antibody binding site specifically binds IL-4, and the third antigen binding site specifically binds IL-13.

9. The antibody as claimed in claim 8, comprising a first, second, third, fourth, and fifth polypeptide chain and wherein the identity of the second, fourth, and fifth polypeptide chains is selected from the group consisting of
(i) the second polypeptide chain comprises SEQ ID NO: 145, the fourth polypeptide chain comprises SEQ ID NO: 196, and the fifth polypeptide chain comprises SEQ ID NO: 103;
(ii) the second polypeptide chain comprises SEQ ID NO: 107, the fourth polypeptide chain comprises SEQ ID NO: 109, and the fifth polypeptide chain comprises SEQ ID NO: 103;
(iii) the second polypeptide chain comprises SEQ ID NO 115, the fourth polypeptide chain comprises SEQ ID NO: 116, and the fifth polypeptide chain comprises SEQ ID NO: 103;
(iv) the second polypeptide chain comprises SEQ ID NO: 121, the fourth polypeptide chain comprises SEQ ID NO: 109, and the fifth polypeptide chain comprises SEQ ID NO: 103;
(v) the second polypeptide chain comprises SEQ ID NO: 125, the fourth polypeptide chain comprises SEQ ID NO: 208, and the fifth polypeptide chain comprises SEQ ID NO: 122;
(vi) the second polypeptide chain comprises SEQ ID NO: 130, the fourth polypeptide chain comprises SEQ ID NO: 27, and the fifth polypeptide chain comprises SEQ ID NO: 122;
(vii) the second polypeptide chain comprises SEQ ID NO: 133, the fourth polypeptide chain comprises SEQ ID NO: 27, and the fifth polypeptide chain comprises SEQ ID NO: 122;
(viii) the second polypeptide chain comprises SEQ ID NO: 144, the fourth polypeptide chain comprises SEQ ID NO: 136, and the fifth polypeptide chain comprises SEQ ID NO: 143;
(ix) the second polypeptide chain comprises SEQ ID NO: 135, the fourth polypeptide chain comprises SEQ ID NO: 136, and the fifth polypeptide chain comprises SEQ ID NO: 122;
(x) the second polypeptide chain comprises SEQ ID NO: 140, the fourth polypeptide chain comprises SEQ ID NO: 27, and the fifth polypeptide chain comprises SEQ ID NO: 122;
(xi) the second polypeptide chain comprises SEQ ID NO: 149, the fourth polypeptide chain comprises SEQ ID NO: 196, and the fifth polypeptide chain comprises SEQ ID NO: 150;
(xii) the second polypeptide chain comprises SEQ ID NO: 151, the fourth polypeptide chain comprises SEQ ID NO: 109, and the fifth polypeptide chain comprises SEQ ID NO: 150;
(xiii) the second polypeptide chain comprises SEQ ID NO 159, the fourth polypeptide chain comprises SEQ ID NO: 109, and the fifth polypeptide chain comprises SEQ ID NO: 150;
(xiv) the second polypeptide chain comprises SEQ ID NO: 162, the fourth polypeptide chain comprises SEQ ID NO: 197, and the fifth polypeptide chain comprises SEQ ID NO: 163;
(xv) the second polypeptide chain comprises SEQ ID NO: 154, the fourth polypeptide chain comprises SEQ ID NO: 196, and the fifth polypeptide chain comprises SEQ ID NO: 155;
(xvi) the second polypeptide chain comprises SEQ ID NO: 156, the fourth polypeptide chain comprises SEQ ID NO: 109, and the fifth polypeptide chain comprises SEQ ID NO: 155;
(xvii) the second polypeptide chain comprises SEQ ID NO: 152, the fourth polypeptide chain comprises SEQ ID NO: 196, and the fifth polypeptide chain comprises SEQ ID NO: 98;
(xviii) the second polypeptide chain comprises SEQ ID NO: 153, the fourth polypeptide chain comprises SEQ ID NO: 109, and the fifth polypeptide chain comprises SEQ ID NO: 98;
(xix) the second polypeptide chain comprises SEQ ID NO: 157, the fourth polypeptide chain comprises SEQ ID NO: 109, and the fifth polypeptide chain comprises SEQ ID NO: 158;
(xx) the second polypeptide chain comprises SEQ ID NO: 160, the fourth polypeptide chain comprises SEQ ID NO: 196, and the fifth polypeptide chain comprises SEQ ID NO: 158;
(xxi) the second polypeptide chain comprises SEQ ID NO: 164, the fourth polypeptide chain comprises SEQ ID NO: 197, and the fifth polypeptide chain comprises SEQ ID NO: 122; and
(xxii) the second polypeptide chain comprises SEQ ID NO: 178, the fourth polypeptide chain comprises SEQ ID NO: 196, and the fifth polypeptide chain comprises SEQ ID NO: 177;
(xxiii) the second polypeptide chain comprises SEQ ID NO: 179, the fourth polypeptide chain comprises SEQ ID NO: 109, and the fifth polypeptide chain comprises SEQ ID NO: 177;
(xxiv) the second polypeptide chain comprises SEQ ID NO 180, the fourth polypeptide chain comprises SEQ ID NO: 109, and the fifth polypeptide chain comprises SEQ ID NO: 122;
(xxv) the second polypeptide chain comprises SEQ ID NO: 183, the fourth polypeptide chain comprises SEQ ID NO: 116, and the fifth polypeptide chain comprises SEQ ID NO: 177;
(xxvi) the second polypeptide chain comprises SEQ ID NO: 125, the fourth polypeptide chain comprises SEQ ID NO: 207, and the fifth polypeptide chain comprises SEQ ID NO: 122;
(xxvii) the second polypeptide chain comprises SEQ ID NO: 125, the fourth polypeptide chain comprises SEQ ID NO: 27, and the fifth polypeptide chain comprises SEQ ID NO: 122.

10. The antibody as claimed in claim 8, comprising a first, second, third, fourth, and fifth polypeptide chain and wherein
(i) the second and fifth polypeptide chains together form a first Fab domain comprising an IL-13 binding site;
(ii) the second and fourth polypeptide chains together form a second Fab domain comprising an IL-4 binding site;
(iii) the first and third polypeptide chains together form a third Fab domain comprising an at least one additional target binding site, and
wherein the second polypeptide chain comprises from N-terminal, SEQ ID NO: 54, SEQ ID NO: 16, an optional linker comprising SEQ ID NO: 10, SEQ ID NO: 22, SEQ ID NO: 6, a CH2 domain, and a CH3 domain; the fourth polypeptide chain comprises SEQ ID NO: 27; and the fifth polypeptide chain comprises SEQ ID NO: 122.

11. An isolated antibody that specifically binds to TSLP, comprising a heavy chain variable region (TSLP-VH) and a light chain variable region (TSLP-VL), comprising
(i) the CDR-H1, CDR-H2, and CDR-H3 sequences of SEQ ID NO: 92, and the CDR-11, CDR-L2, and CDR-L3 sequences of SEQ ID NO: 94;
(ii) the CDR-H1, CDR-H2, and CDR-H3 sequences of SEQ ID NO: 92, and the CDR-11, CDR-L2, and CDR-L3 sequences of SEQ ID NO: 93;
(iii) the CDR-H1, CDR-H2, and CDR-H3 sequences of SEQ ID NO: 92, and the CDR-11, CDR-L2, and CDR-L3 sequences of SEQ ID NO: 213;
(iv) the CDR-H1, CDR-H2, and CDR-H3 sequences of SEQ ID NO: 92, and the CDR-11, CDR-L2, and CDR-L3 sequences of SEQ ID NO: 214;
(v) the CDR-H1, CDR-H2, and CDR-H3 sequences of SEQ ID NO: 221, and the CDR-11, CDR-L2, and CDR-L3 sequences of SEQ ID NO: 213;
(vi) the CDR-H1, CDR-H2, and CDR-H3 sequences of SEQ ID NO: 221, and the CDR-11, CDR-L2, and CDR-L3 sequences of SEQ ID NO: 94; or
(vii) the CDR-H1, CDR-H2, and CDR-H3 sequences of SEQ ID NO: 221, and the CDR-11, CDR-L2, and CDR-L3 sequences of SEQ ID NO: 223.

12. The antibody as claimed in claim 11, comprising
(i) a TSLP-VH sequence of SEQ ID NO: 92, and a TSLP-VL of SEQ ID NO: 94;
(ii) a TSLP-VH sequence of SEQ ID NO: 92, and a TSLP-VL of SEQ ID NO: 93;
(iii) a TSLP-VH sequence of SEQ ID NO: 92, and a TSLP-VL of SEQ ID NO: 213;
(iv) a TSLP-VH sequence of SEQ ID NO: 92, and a TSLP-VL of SEQ ID NO: 214;
(v) the TSLP-VH sequence of SEQ ID NO: 221, and the TSLP-VL of SEQ ID NO: 215;
(vi) the TSLP-VH sequence of SEQ ID NO: 221, and the TSLP-VL of SEQ ID NO: 99;
(vii) the TSLP-VH sequence of SEQ ID NO: 221, and the TSLP-VL of SEQ ID NO: 224;
(viii) a TSLP-VH sequence encoded by a nucleic acid sequence of SEQ ID NO: 204; and a TSLP-VL sequence encoded by a nucleic acid sequence of SEQ ID NO: 205,
(ix) a TSLP-VH sequence encoded by a nucleic acid sequence of SEQ ID NO: 204; and a TSLP-VL sequence encoded by a nucleic acid sequence of SEQ ID NO: 217;
(x) a TSLP-VH sequence encoded by a nucleic acid sequence of SEQ ID NO: 204; and a TSLP-VL sequence encoded by a nucleic acid sequence of SEQ ID NO: 218;
(xi) a TSLP-VH bearing polypeptide sequence encoded by a nucleic acid sequence of SEQ ID NO: 192; and a TSLP-VL bearing polypeptide sequence encoded by a nucleic acid sequence of SEQ ID NO: 193;
(xii) a TSLP-VH sequence encoded by the plasmid deposited at the ATCC and having ATCC Accession No. PTA-127200, and a TSLP-VL sequence encoded by the plasmid deposited at the ATCC and having ATCC Accession No. PTA-127199; or
(xiii) a TSLP-VH bearing polypeptide sequence encoded by the plasmid deposited at the ATCC and having ATCC Accession No. PTA-127202, and a TSLP-VL bearing polypeptide sequence encoded by the plasmid deposited at the ATCC and having ATCC Accession No. PTA-127201.

13. The antibody of claim 11, wherein the antibody is characterized by one or more of
(i) an IC₅₀ of less than 10 pM in a TARC production bioassay in human peripheral blood monocytes;
(ii) a melting temperature of 68° C.;
(iii) wherein the pH3.4 hold Δ% HMMS is less than 5, such that the pH3.4 hold Δ% HMMS is defined as the difference between the percentage of high molecular weight species due to degradation after 5 hours of incubation of the antibody at room temperature at pH3.4 and the percentage of high molecular weight species due to degradation after 5 hours of incubation of the antibody at room temperature at pH 7.2;
(iv) an anti-TSLP bioactivity of an IC₅₀ of less than 10 pM as measured a TARC production bioassay in human primary PBMCs;
(v) a viscosity of 20 cP at a concentration of at least 100 mg/mL in a buffer of 20 mM Histidine, 8.5% sucrose, 0.05 mg/mL EDTA pH 6.0;
(vi) a score of less than 2% high molecular mass species when determined by analytical size-exclusion chromatography (aSEC);
(vii) a score of less than 12 in an affinity-capture self-interaction nanoparticle spectroscopy (AC SINS) assay;
(viii) an IC₅₀ of less than 15 pM in a human TSLP neutralization in a TARC production bioassay in human primary PBMCs;
(ix) an IC₅₀ of less than 35 pM in a cynomolgus TSLP neutralization assay.

14. The isolated antibody of claim 11, wherein the antibody also specifically binds to IL-4 through an IL-4 binding domain, and specifically binds to IL-13 through an IL-13 binding domain.

15. The antibody of claim 14, wherein
(i) the IL-4 binding domain comprises a heavy chain variable region (IL4-VH) and a light chain variable region (IL4-VL), wherein the CDR-H1 comprises the amino acid sequence of SEQ ID NO: 18; the CDR-H2 comprises the amino acid sequence of SEQ ID NO: 2; the CDR-H3 comprises the amino acid sequence of SEQ ID NO: 3; the CDR-L1 comprises the amino acid sequence of SEQ ID NO: 24; the CDR-L2 comprises the amino acid sequence of SEQ ID NO: 12, and the CDR-L3 comprises the amino acid sequence of SEQ ID NO: 25, and
(ii) the IL-13 binding domain comprises a heavy chain variable region (IL13-VH) and a light chain variable region (IL13-VL), wherein the CDR-H1 comprises the amino acid sequence of SEQ ID NO: 41; the CDR-H2 comprises the amino acid sequence of SEQ ID NO: 42; the CDR-H3 comprises the amino acid sequence of SEQ ID NO:-50; the CDR-L1 comprises the amino acid sequence of SEQ ID NO: 53; the CDR-L2 comprises the amino acid sequence of SEQ ID NO: 37, and the CDR-L3 comprises the amino acid sequence of SEQ ID NO: 38; and
wherein:
(iii) the TSLP binding domain comprises a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 88, and the CDR-L3 comprises the amino acid sequence of SEQ ID NO: 90; OR
(iv) the TSLP binding domain comprises a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 211; OR
(v) the TSLP binding domain comprises a heavy chain variable region (TSLP-VH) and a light chain variable region (TSLP-VL), wherein the CDR-H1 comprises the amino acid sequence of SEQ ID NO: 82; the CDR-H2 comprises the amino acid sequence of SEQ ID NO: 83; the CDR-H3 comprises the amino acid sequence of SEQ ID NO: 85; the CDR-L1 comprises the amino acid sequence of SEQ ID NO: 86; the CDR-L2 comprises the amino acid sequence of SEQ ID NO: 87, and the CDR-L3 comprises the amino acid sequence of SEQ ID NO: 212.

16. The antibody of claim 14, wherein the antibody comprises a first, second, third, fourth, and fifth polypeptide chain, such that the second and fifth polypeptide chains together form a first Fab domain comprising a first antigen binding site; the second and fourth polypeptide chains together form a second Fab domain comprising a second antigen binding site; and the first and third polypeptide chains together form a third Fab domain comprising a third antigen binding site, and wherein the identity of the first, second, third, fourth, and fifth polypeptide chains is selected from the group consisting of
(i) the first polypeptide chain comprises SEQ ID NO: 165, the second polypeptide chain comprises SEQ ID NO: 130, the third polypeptide chain comprises SEQ ID NO: 99, the fourth polypeptide chain comprises SEQ ID NO: 27, and the fifth polypeptide chain comprises SEQ ID NO: 122;
(ii) the first polypeptide chain comprises SEQ ID NO: 146, the second polypeptide chain comprises SEQ ID NO: 149, the third polypeptide chain comprises SEQ ID NO: 109, the fourth polypeptide chain comprises SEQ ID NO: 196, and the fifth polypeptide chain comprises SEQ ID NO: 150;
(iii) the first polypeptide chain comprises SEQ ID NO: 112, the second polypeptide chain comprises SEQ ID NO: 151, the third polypeptide chain comprises SEQ ID NO: 196, the fourth polypeptide chain comprises SEQ ID NO: 109, and the fifth polypeptide chain comprises SEQ ID NO: 150;
(iv) the first polypeptide chain comprises SEQ ID NO: 112, the second polypeptide chain comprises SEQ ID NO 159, the third polypeptide chain comprises SEQ ID NO: 196, the fourth polypeptide chain comprises SEQ ID NO: 109, and the fifth polypeptide chain comprises SEQ ID NO: 150;
(v) the first polypeptide chain comprises SEQ ID NO: 161, the second polypeptide chain comprises SEQ ID NO: 162, the third polypeptide chain comprises SEQ ID NO: 98, the fourth polypeptide chain comprises SEQ ID NO: 197, and the fifth polypeptide chain comprises SEQ ID NO: 163;
(vi) the first polypeptide chain comprises SEQ ID NO: 146, the second polypeptide chain comprises SEQ ID NO: 154, the third polypeptide chain comprises SEQ ID NO: 109, the fourth polypeptide chain comprises SEQ ID NO: 196, and the fifth polypeptide chain comprises SEQ ID NO: 155;
(vii) the first polypeptide chain comprises SEQ ID NO: 112, the second polypeptide chain comprises SEQ ID NO: 156, the third polypeptide chain comprises SEQ ID NO: 196, the fourth polypeptide chain comprises SEQ ID NO: 109, and the fifth polypeptide chain comprises SEQ ID NO: 155;
(viii) the first polypeptide chain comprises SEQ ID NO: 146, the second polypeptide chain comprises SEQ ID NO: 152, the third polypeptide chain comprises SEQ ID NO: 109, the fourth polypeptide chain comprises SEQ ID NO: 196, and the fifth polypeptide chain comprises SEQ ID NO: 98;
(ix) the first polypeptide chain comprises SEQ ID NO: 112, the second polypeptide chain comprises SEQ ID NO: 153, the third polypeptide chain comprises SEQ ID NO: 196, the fourth polypeptide chain comprises SEQ ID NO: 109, and the fifth polypeptide chain comprises SEQ ID NO: 98;
(x) the first polypeptide chain comprises SEQ ID NO: 112, the second polypeptide chain comprises SEQ ID NO: 157, the third polypeptide chain comprises SEQ ID NO: 196, the fourth polypeptide chain comprises SEQ ID NO: 109, and the fifth polypeptide chain comprises SEQ ID NO: 158;
(xi) the first polypeptide chain comprises SEQ ID NO: 146, the second polypeptide chain comprises SEQ ID NO: 160, the third polypeptide chain comprises SEQ ID NO: 109, the fourth polypeptide chain comprises SEQ ID NO: 196, and the fifth polypeptide chain comprises SEQ ID NO: 158; and
(xii) the first polypeptide chain comprises SEQ ID NO: 161, the second polypeptide chain comprises SEQ ID NO: 164, the third polypeptide chain comprises SEQ ID NO: 98, the fourth polypeptide chain comprises SEQ ID NO: 197, and the fifth polypeptide chain comprises SEQ ID NO: 122;
(xiii) the first polypeptide chain comprises SEQ ID NO: 165, the second polypeptide chain comprises SEQ ID NO: 130, the third polypeptide chain comprises SEQ ID NO: 215, the fourth polypeptide chain comprises SEQ ID NO: 27, and the fifth polypeptide chain comprises SEQ ID NO: 122;
(xiv) the first polypeptide chain comprises SEQ ID NO: 165, the second polypeptide chain comprises SEQ ID NO: 130, the third polypeptide chain comprises SEQ ID NO: 216, the fourth polypeptide chain comprises SEQ ID NO: 27, and the fifth polypeptide chain comprises SEQ ID NO: 122;
(xv) the first polypeptide chain comprises a sequence encoded by a nucleic acid according to SEQ ID NO: 192, the second polypeptide chain comprises a sequence encoded by a nucleic acid according to SEQ ID NO: 188, the third polypeptide chain comprises a sequence encoded by a nucleic acid according to SEQ ID NO: 193, the fourth polypeptide chain comprises a sequence encoded by a nucleic acid according to SEQ ID NO: 187, and the fifth polypeptide chain comprises a sequence encoded by a nucleic acid according to SEQ ID NO: 122; and
(xvi) the first polypeptide chain comprises a sequence encoded by a nucleic acid corresponding to the ATCC deposit PTA-127202, the second polypeptide chain comprises a sequence encoded by a nucleic acid corresponding to the ATCC deposit PTA-127192, the third polypeptide chain comprises a sequence encoded by a nucleic acid corresponding to the ATCC deposit PTA-127201, the fourth polypeptide chain comprises a sequence encoded by a nucleic acid corresponding to the ATCC deposit PTA-127194, and the fifth polypeptide chain comprises a sequence encoded by a nucleic acid corresponding to the ATCC deposit PTA-127193.

17. An isolated antibody that specifically binds to p40 through a p40 binding domain and wherein the antibody further comprises an IL-4 binding domain that specifically binds to IL-4, and an IL-13 binding domain that specifically binds to IL-13, and wherein the p40 binding domain comprises
(i) a heavy chain variable region (p40-VH) and a light chain variable region (p40-VL), comprising the CDR-H1, CDR-H2, and CDR-H3 sequence of SEQ ID NO: 169, and the CDR-L1, CDR-L2, and CDR-L3 sequences of SEQ ID NO: 175;
(ii) a p40-VH comprising a sequence according to SEQ ID NO: 169, and a p40-VL according to SEQ ID NO: 175;
(iii) a p40-VH bearing polypeptide comprising a sequence according to SEQ ID NO: 186, and a p40-VL bearing polypeptide comprising a sequence according to SEQ ID NO: 176
(iv) a p40-VH according to SEQ ID NO: 206, and a p40-VL according to SEQ ID NO: 207;
(v) a p40-VH bearing polypeptide according to SEQ ID NO: 194, and a p40-VH bearing polypeptide according to SEQ ID NO: 195;
(vi) a p40-VH comprising a sequence encoded by a nucleic acid corresponding to the ATCC deposit PTA-127206, and a p40-VL comprising a sequence encoded by a nucleic acid corresponding to the ATCC deposit PTA-127205; or
(vii) a p40-VH bearing polypeptide comprising a sequence encoded by a nucleic acid corresponding to the ATCC deposit PTA-127204, and a p40-VL bearing polypeptide comprising a sequence encoded by a nucleic acid corresponding to the ATCC deposit PTA-127203.

18. The antibody of claim 17, wherein
(i) the p40 binding domain comprises a heavy chain variable region (p40-VH) and a light chain variable region (p40-VL), wherein the CDR-H1 of the p40 binding domain comprises the amino acid sequence of SEQ ID NO: 166; the CDR-H2 of the p40 binding domain comprises the amino acid sequence of SEQ ID NO: 167; the CDR-H3 of the p40 binding domain comprises the amino acid sequence of SEQ ID NO: 168; the CDR-L1 of the p40 binding domain comprises the amino acid sequence of SEQ ID NO: 171; the CDR-L2 of the p40 binding domain comprises the amino acid sequence of SEQ ID NO: 172, and the CDR-L3 of the p40 binding domain comprises the amino acid sequence of SEQ ID NO: 173; and
(ii) the IL-4 binding domain comprises a heavy chain variable region (IL4-VH) and a light chain variable region (IL4-VL), wherein the CDR-H1 of the IL-4 binding domain comprises the amino acid sequence of SEQ ID NO: 18; the CDR-H2 of the IL-4 binding domain comprises the amino acid sequence of SEQ ID NO: 2; the CDR-H3 of the IL-4 binding domain comprises the amino acid sequence of SEQ ID NO: 3; the CDR-L1 of the IL-4 binding domain comprises the amino acid sequence of SEQ ID NO: 24; the CDR-L2 of the IL-4 binding domain comprises the amino acid sequence of SEQ ID NO: 12, and the CDR-L3 of the IL-4 binding domain comprises the amino acid sequence of SEQ ID NO: 25, and
(iii) the IL-13 binding domain comprises a heavy chain variable region (IL13-VH) and a light chain variable region (IL13-VL), wherein the CDR-H1 of the IL-13 binding domain comprises the amino acid sequence of SEQ ID NO: 41; the CDR-H2 of the IL-13 binding domain comprises the amino acid sequence of SEQ ID NO: 42; the CDR-H3 of the IL-13 binding domain comprises the amino acid sequence of SEQ ID NO:-50; the CDR-L1 of the IL-13 binding domain comprises the amino acid sequence of SEQ ID NO: 53; the CDR-L2 of the IL-13 binding domain comprises the amino acid sequence of SEQ ID NO: 37, and the CDR-L3 of the IL-13 binding domain comprises the amino acid sequence of SEQ ID NO: 38.

19. The antibody of claim 18, wherein the antibody comprises a first, second, third, fourth, and fifth polypeptide chain, such that the second and fifth polypeptide chains together form a first Fab domain comprising a first antigen binding site; the second and fourth polypeptide chains together form a second Fab domain comprising a second antigen binding site; the first and third polypeptide chains together form a third Fab domain comprising a third antigen binding site, and wherein the identity of the first, second, third, fourth, and fifth polypeptide chains is selected from the group consisting of
(i) the first polypeptide chain comprises SEQ ID NO: 186, the second polypeptide chain comprises SEQ ID NO: 130, the third polypeptide chain comprises SEQ ID NO: 176, the fourth polypeptide chain comprises SEQ ID NO: 27, and the fifth polypeptide chain comprises SEQ ID NO: 122;
(ii) the first polypeptide chain comprises SEQ ID NO: 146, the second polypeptide chain comprises SEQ ID NO: 178, the third polypeptide chain comprises SEQ ID NO: 109, the fourth polypeptide chain comprises SEQ ID NO: 196, and the fifth polypeptide chain comprises SEQ ID NO: 177;
(iii) the first polypeptide chain comprises SEQ ID NO: 112, the second polypeptide chain comprises SEQ ID NO: 179, the third polypeptide chain comprises SEQ ID NO: 196, the fourth polypeptide chain comprises SEQ ID NO: 109, and the fifth polypeptide chain comprises SEQ ID NO: 177;
(iv) the first polypeptide chain comprises SEQ ID NO: 181, the second polypeptide chain comprises SEQ ID NO 180, the third polypeptide chain comprises SEQ ID NO: 182, the fourth polypeptide chain comprises SEQ ID NO: 109, and the fifth polypeptide chain comprises SEQ ID NO: 122;
(v) the first polypeptide chain comprises SEQ ID NO: 118, the second polypeptide chain comprises SEQ ID NO: 183, the third polypeptide chain comprises SEQ ID NO: 120, the fourth polypeptide chain comprises SEQ ID NO: 116, and the fifth polypeptide chain comprises SEQ ID NO: 177;
(vi) the first polypeptide chain comprises SEQ ID NO: 185, the second polypeptide chain comprises SEQ ID NO: 125, the third polypeptide chain comprises SEQ ID NO: 176, the fourth polypeptide chain comprises SEQ ID NO: 207, and the fifth polypeptide chain comprises SEQ ID NO: 122;
(vii) the first polypeptide chain comprises SEQ ID NO: 185, the second polypeptide chain comprises SEQ ID NO: 125, the third polypeptide chain comprises SEQ ID NO: 176, the fourth polypeptide chain comprises SEQ ID NO: 27, and the fifth polypeptide chain comprises SEQ ID NO: 122;
(viii) the first polypeptide chain comprises a sequence encoded by a nucleic acid according to SEQ ID NO: 194, the second polypeptide chain comprises a sequence encoded by a nucleic acid according to SEQ ID NO: 188, the third polypeptide chain comprises a sequence encoded by a nucleic acid according to SEQ ID NO: 195, the fourth polypeptide chain comprises a sequence encoded by a nucleic acid according to SEQ ID NO: 187, and the fifth polypeptide chain comprises a sequence encoded by a nucleic acid according to SEQ ID NO: 122; and
(ix) the first polypeptide chain comprises a sequence encoded by a nucleic acid corresponding to the ATCC deposit PTA-127204, the second polypeptide chain comprises a sequence encoded by a nucleic acid corresponding to the ATCC deposit PTA-127192, the third polypeptide chain comprises a sequence encoded by a nucleic acid corresponding to the ATCC deposit PTA-127203, the fourth polypeptide chain comprises a sequence encoded by a nucleic acid corresponding to the ATCC deposit PTA-127194, and the fifth polypeptide chain comprises a sequence encoded by a nucleic acid corresponding to the ATCC deposit PTA-127193.

20. The antibody of claim 17, wherein the antibody is characterized by one or more of:
(i) a viscosity of less than 20 cP at concentrations of at least 100 mg/mL in a buffer of 20 mM Histidine, 8.5% sucrose, 0.05 mg/mL EDTA pH 6.0;
(ii) a viscosity of less than 12 cP at concentrations of at least 50 mg/m in a buffer of 20 mM Histidine, 8.5% sucrose, 0.05 mg/mL EDTA pH 6.0;
(iii) a terminal half-life of at least 12 days in cynomolgus monkeys;
(iv) a terminal half-life of at least 18 days in TG-32 mice;
(v) the antibody binds human IL-4 with an affinity constant of less than 220 pM as measured by SPR;
(vi) the antibody binds human IL-13 with an affinity constant of less than 220 pM as measured by SPR;
(vii) the antibody binds human IL-12 with an affinity constant of less than 130 pM as measured by SPR;
(viii) the antibody binds human IL-23 with an affinity constant of less than 100 pM as measured by SPR;
(ix) the antibody binds to human IL-4 with a binding affinity of less than 1 pM, as measured by KinExA in a fixed antigen assay in PBS;
(x) the antibody binds to cynomolgus IL-13 with a binding affinity of less than 2 pM, as measured by KinExA in a fixed antigen assay in PBS;
(xi) an IC₅₀ of less than 12 pM as measured in a human monocyte assay for neutralization of IL-4 induction of CD23;
(xii) an IC₅₀ of less than 12 pM as measured in a human monocyte assay for neutralization of cynomolgus monkey IL-4 induction of CD23;
(xiii) an IC₅₀ of less than 12 pM as measured in a human monocyte assay for neutralization of cynomolgus monkey IL-13 induction of CD23;
(xiv) an IC₅₀ of less than 45 pM as measured in a human monocyte assay for neutralization of IL-13 induction of CD23;
(xv) an IC₅₀ of less than 600 pM in a human IL-12 neutralization Kit-225 assay in human peripheral blood monocytes;
(xvi) an IC₅₀ of less than 2100 pM in a cynomolgus IL-23 Kit-225 neutralization assay in human peripheral blood monocytes;
(xvii) an IC₅₀ of less than 400 pM in a human IL-12 neutralization assay in human whole blood;
(xviii) an IC₅₀ of less than 10,000 pM in a cynomolgus IL-23 neutralization assay in human whole blood.

21. An isolated antibody that specifically binds to IL-33, comprising a heavy chain variable region (IL33-VH) and a light chain variable region (IL33-VL), comprising
(i) the CDR-H1, CDR-H2, and CDR-H3 sequences of SEQ ID NO: 73, and the CDR-11, CDR-L2, and CDR-L3 sequences of SEQ ID NO: 78;
(ii) the CDR-H1, CDR-H2, and CDR-H3 sequences of SEQ ID NO: 63, and the CDR-11, CDR-L2, and CDR-L3 sequences of SEQ ID NO: 71; or
(iii) the CDR-H1, CDR-H2, and CDR-H3 sequences of SEQ ID NO: 80, and the CDR-11, CDR-L2, and CDR-L3 sequences of SEQ ID NO: 81.

22. The antibody of claim 21, comprising
(i) an IL33-VH sequence of SEQ ID NO: 73, and an IL33-VL of SEQ ID NO: 78; or
(ii) an IL33-VH sequence of SEQ ID NO: 63, and an IL33-VL of SEQ ID NO: 71; or
(iii) an IL33-VH sequence of SEQ ID NO: 80, and an IL33-VL of SEQ ID NO: 81; or
(iv) an IL33-VH sequence encoded by a nucleic acid sequence of SEQ ID NO: 202, and an IL33-VL sequence encoded by a nucleic acid sequence of SEQ ID NO 203;
(v) an IL33-VH bearing polypeptide sequence encoded by a nucleic acid sequence of SEQ ID NO: 190, and an IL33-VL bearing polypeptide sequence encoded by a nucleic acid sequence of SEQ ID NO 191;
(vi) an IL33-VH sequence encoded by the plasmid deposited at the ATCC and having ATCC Accession No. PTA-127210 and an IL33-VL sequence encoded by the plasmid deposited at the ATCC and having ATCC Accession No. PTA-127209; or
(vii) an IL33-VH bearing polypeptide sequence encoded by the plasmid deposited at the ATCC and having ATCC Accession No. PTA-127208, and an IL33-VL bearing polypeptide encoded by the plasmid deposited at the ATCC and having ATCC Accession No. PTA-127207.

23. The antibody of claim 21, wherein the antibody further specifically binds to IL-4 through an IL-4 binding domain, and further specifically binds to IL-13, through an IL-13 binding domain.

24. The antibody of claim 23, wherein
(i) the IL-33 binding domain comprises a heavy chain variable region (IL33-VH) and a light chain variable region (IL33-VL), wherein the CDR-H1 of the IL-33 binding domain comprises the amino acid sequence of SEQ ID NO: 60; the CDR-H2 of the IL-33 binding domain comprises the amino acid sequence of SEQ ID NO: 61; the CDR-H3 of the IL-33 binding domain comprises the amino acid sequence of SEQ ID NO: 72; the CDR-L1 of the IL-33 binding domain comprises the amino acid sequence of SEQ ID NO: 75; the CDR-L2 of the IL-33 binding domain comprises the amino acid sequence of SEQ ID NO: 76, and the CDR-L3 of the IL-33 binding domain comprises the amino acid sequence of SEQ ID NO: 77; and
(ii) the IL-4 binding domain comprises a heavy chain variable region (IL4-VH) and a light chain variable region (IL4-VL), wherein the CDR-H1 of the IL-4 binding domain comprises the amino acid sequence of SEQ ID NO: 18; the CDR-H2 of the IL-4 binding domain comprises the amino acid sequence of SEQ ID NO: 2; the CDR-H3 of the IL-4 binding domain comprises the amino acid sequence of SEQ ID NO: 3; the CDR-L1 of the IL-4 binding domain comprises the amino acid sequence of SEQ ID NO: 24; the CDR-L2 of the IL-4 binding domain comprises the amino acid sequence of SEQ ID NO: 12, and the CDR-L3 of the IL-4 binding domain comprises the amino acid sequence of SEQ ID NO: 25, and
(iii) the IL-13 binding domain comprises a heavy chain variable region (IL13-VH) and a light chain variable region (IL13-VL), wherein the CDR-H1 of the IL-13 binding domain comprises the amino acid sequence of SEQ ID NO: 41; the CDR-H2 of the IL-13 binding domain comprises the amino acid sequence of SEQ ID NO: 42; the CDR-H3 of the IL-13 binding domain comprises the amino acid sequence of SEQ ID NO:-50; the CDR-L1 of the IL-13 binding domain comprises the amino acid sequence of SEQ ID NO: 53; the CDR-L2 of the IL-13 binding domain comprises the amino acid sequence of SEQ ID NO: 37, and the CDR-L3 of the IL-13 binding domain comprises the amino acid sequence of SEQ ID NO: 38.

25. The antibody of claim 23, wherein the IL-4 binding domain is characterized by one or more of the following:
(i) binding human IL-4 with a K_{D} less than a value selected from the group consisting of about, 10 pM, 5 pM, 1 pM, and 800 fM;
(ii) binding cynomolgus IL-4;
(iii) not binding IL-4 from one or more selected from the groups consisting of dog, sheep, rabbit, rat, and mouse;
(iv) the binding K_{D} to cynomolgus IL-4 is within 1 order of magnitude of the binding K_{D} of the antibody to human IL-4;
(v) an IC₅₀ of less than 10 pM in a human monocyte assay for neutralization of IL-4 induction of CD23;
(vi) a viscosity of 20 cP or less at 25° C. at a concentration of 80 mg/mL in a Histidine-sucrose pH 5.8 buffer;
(vii) a lysine at residue 93 in the light chain.

26. The antibody of claim 23, characterized by one or more of the following:
(i) the antibody binds human IL-13 with a K_{D} less than a value selected from the group consisting of 10 nM, 5 nM, 2 nM, 1 nM, 900 pM, 800 pM, 700 pM, 600 pM, 500 pM, 400 pM, 300 pM, 250 pM, 200 pM, 150 pM, 100 pM, and 60 pM;
(ii) the antibody binds cynomolgus IL-13, optionally within 1 order of magnitude of the binding K_{D} of the antibody to human IL-13;
(iii) the IL-13 IC₅₀ is less than 100 pM as measured by neutralization of IL-13 pSTAT6 phosphorylation in HT-29 cells;
(iv) the IL-13 IC₅₀ is less than 20 pM as measured in a human monocyte assay for neutralization of IL-13 induction of CD23;
(v) the antibody has a terminal half-life of at least 14 days in cynomolgus monkeys;
(vi) the antibody has a terminal half-life of at least 18 days in TG32 mice;
(vii) the antibody does not bind IL-13 from one or more species selected from the group consisting of dog, rabbit, and mouse.

27. The antibody of claim 23, wherein the antibody comprises a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site, and
wherein the identity of the first, second, third, fourth, and fifth polypeptide chains is selected from the group consisting of
(i) the first polypeptide chain comprises SEQ ID NO: 132, the second polypeptide chain comprises SEQ ID NO: 130, the third polypeptide chain comprises SEQ ID NO: 79, the fourth polypeptide chain comprises SEQ ID NO: 27, and the fifth polypeptide chain comprises SEQ ID NO: 122;
(ii) the first polypeptide chain comprises SEQ ID NO: 146, the second polypeptide chain comprises SEQ ID NO: 145, the third polypeptide chain comprises SEQ ID NO: 109, the fourth polypeptide chain comprises SEQ ID NO: 196, and the fifth polypeptide chain comprises SEQ ID NO: 103;
(iii) the first polypeptide chain comprises SEQ ID NO: 112, the second polypeptide chain comprises SEQ ID NO: 107, the third polypeptide chain comprises SEQ ID NO: 196, the fourth polypeptide chain comprises SEQ ID NO: 109, and the fifth polypeptide chain comprises SEQ ID NO: 103;
(iv) the first polypeptide chain comprises SEQ ID NO: 118, the second polypeptide chain comprises SEQ ID NO 115, the third polypeptide chain comprises SEQ ID NO: 119, the fourth polypeptide chain comprises SEQ ID NO: 116, and the fifth polypeptide chain comprises SEQ ID NO: 103;
(v) the first polypeptide chain comprises SEQ ID NO: 118, the second polypeptide chain comprises SEQ ID NO: 115, the third polypeptide chain comprises SEQ ID NO: 120, the fourth polypeptide chain comprises SEQ ID NO: 116, and the fifth polypeptide chain comprises SEQ ID NO: 103;
(vi) the first polypeptide chain comprises SEQ ID NO: 209, the second polypeptide chain comprises SEQ ID NO: 121, the third polypeptide chain comprises SEQ ID NO: 196, the fourth polypeptide chain comprises SEQ ID NO: 109, and the fifth polypeptide chain comprises SEQ ID NO: 103;
(vii) the first polypeptide chain comprises SEQ ID NO: 128, the second polypeptide chain comprises SEQ ID NO: 125, the third polypeptide chain comprises SEQ ID NO: 79, the fourth polypeptide chain comprises SEQ ID NO: 208, and the fifth polypeptide chain comprises SEQ ID NO: 122;
(viii) the first polypeptide chain comprises SEQ ID NO: 134, the second polypeptide chain comprises SEQ ID NO: 133, the third polypeptide chain comprises SEQ ID NO: 79, the fourth polypeptide chain comprises SEQ ID NO: 27, and the fifth polypeptide chain comprises SEQ ID NO: 122;
(ix) the first polypeptide chain comprises SEQ ID NO: 121, the second polypeptide chain comprises SEQ ID NO: 144, the third polypeptide chain comprises SEQ ID NO: 196, the fourth polypeptide chain comprises SEQ ID NO: 136, and the fifth polypeptide chain comprises SEQ ID NO: 143;
(x) the first polypeptide chain comprises SEQ ID NO: 137, the second polypeptide chain comprises SEQ ID NO: 135, the third polypeptide chain comprises SEQ ID NO: 138, the fourth polypeptide chain comprises SEQ ID NO: 136, and the fifth polypeptide chain comprises SEQ ID NO: 122;
(xi) the first polypeptide chain comprises SEQ ID NO: 142, the second polypeptide chain comprises SEQ ID NO: 140, the third polypeptide chain comprises SEQ ID NO: 79, the fourth polypeptide chain comprises SEQ ID NO: 27, and the fifth polypeptide chain comprises SEQ ID NO: 122;
(xii) the first polypeptide chain comprises a sequence encoded by a nucleic acid according to SEQ ID NO: 190, the second polypeptide chain comprises a sequence encoded by a nucleic acid according to SEQ ID NO: 188, the third polypeptide chain comprises a sequence encoded by a nucleic acid according to SEQ ID NO: 191, the fourth polypeptide chain comprises a sequence encoded by a nucleic acid according to SEQ ID NO: 187, and the fifth polypeptide chain comprises a sequence encoded by a nucleic acid according to SEQ ID NO: 122; and
(xiii) wherein the first polypeptide chain comprises a sequence encoded by the plasmid deposited at the ATCC and having ATCC Accession No. PTA-127208; the second polypeptide chain comprises a sequence encoded by the plasmid deposited at the ATCC and having ATCC Accession No. PTA-127192; the third polypeptide chain comprises a sequence encoded by the plasmid deposited at the ATCC and having ATCC Accession No. PTA-127207; the fourth polypeptide chain comprises a sequence encoded by the plasmid deposited at the ATCC and having ATCC Accession No. PTA-127194; and the fifth polypeptide chain comprises a sequence encoded by the plasmid deposited at the ATCC and having ATCC Accession No. PTA-127193.

28. The antibody of claim 23, wherein the antibody is characterized by one or more of:
(i) binding human IL-33 with a K_{D} less than a value selected from the group consisting of 100 pM, 50 pM, 40 pM, 30 pM, 25 pM, 20 pM, 15 pM, and 10 pM, 5 pM, 2 pM, 1 pM, 750 fM, 500 fM, and 250 fM;
(ii) an IC₅₀ of less than 20 pM in a HEK Blue® IL-33 neutralization SEAP assay.
(iii) binding cynomolgus IL-33;
(iv) the binding K_{D} of the antibody to cynomolgus IL-33 is within 3 orders of magnitude of the binding K_{D} of the antibody to human IL-33;
(v) a viscosity of less than 20 cP at concentrations of at least 50 mg/mL in a buffer of 20 mM Histidine, 8.5% sucrose, 0.05 mg/mL EDTA pH 6.0;
(vi) a viscosity of less than 15 cP at concentrations of at least 90 mg/mL in a buffer of 20 mM Histidine, 8.5% sucrose, 0.05 mg/mL EDTA pH 6.0;
(vii) a terminal half-life of at least 12 day in cynomolgus monkeys;
(viii) a terminal half-life of at least 16 days in TG32 mice;
(ix) binds one or both of human IL-4 and human IL-13 with a binding affinity of less than 220 nM as measured by SPR;
(x) binds one or both of human IL-4 and human IL-13 with a binding affinity of less than 1 pM, as measured by KinExA in a fixed antigen assay in PBS;
(xi) an IC₅₀ of less than 20 nM in a human monocyte assay for neutralization of IL-4 induction of CD23;
(xii) an IC₅₀ of less than 20 nM in a human monocyte assay for neutralization of IL-13 induction of CD23;
(xiii) an IC₅₀ of less than 30 nM in a wild-type IL-33 neutralization HEK-Blue SEAP assay;
(xiv) an IC₅₀ of less than 15 pM in a recombinant constitutively active IL-33 neutralization HEK-Blue SEAP assay.

29. An antibody comprising a first, second, third, fourth, and fifth polypeptide chain, such that
(i) the second and fifth polypeptide chains together form a first Fab domain comprising a first antigen binding site;
(ii) the second and fourth polypeptide chains together form a second Fab domain comprising a second antigen binding site;
(iii) the first and third polypeptide chains together form a third Fab domain comprising a third antigen binding site;
wherein the first and second polypeptide chains associate together to form an antibody comprising two arms; a dual Fab arm comprising the first Fab domain and the second Fab domain, and a single Fab arm comprising the third Fab domain; and wherein
the first Fab comprises a first antigen associated VH (VH-1), a first antigen associated VL (VL-1), a first antigen associated CL (CL-1), and a first antigen associated CH1 (CH1-1), and wherein the C-terminus of the VH-1 is covalently fused by a peptide bond to the N-terminus of the CH1-1, and wherein the C-terminus of the VL-1 is covalently fused by a peptide bond to the N-terminus of the CL-1, and
wherein the second Fab comprises a second antigen associated VH (VH-2), a second antigen associated VL (VL-2), a second antigen associated CL (CL-2), and a second antigen associated CH1 (CH1-2), and wherein the C-terminus of the VH-2 is covalently fused by a peptide bond to the N-terminus of the CH1-2, and wherein the C-terminus of the VL-2 is covalently fused by a peptide bond to the N-terminus of the CL-2, and
wherein the third Fab comprises a third antigen associated VH (VH-3), a first antigen associated VL (VL-3), a first antigen associated CL (CL-3), and a first antigen associated CH1 (CH1-3), and wherein the C-terminus of the VH-3 is covalently fused by a peptide bond to the N-terminus of the CH1-3, and wherein the C-terminus of the VL-3 is covalently fused by a peptide bond to the N-terminus of the CL-3, and
wherein the first polypeptide comprises from N-terminus to C-terminus, (VH-3)-(CH1-3)-(first hinge)-(first CH2)-(first CH3); the second polypeptide comprises from N-terminus to C-terminus, (VL-1)-(CL-1)-(linker)-(VH-2)-(CH1-2)-(second hinge)-(second CH2)-(second CH3); the third polypeptide comprises (VL-3)-(CL-3); the fourth polypeptide comprises (VL-2)-(CL-2); and the fifth polypeptide comprises from N-terminus to C-terminus, (VH1)-(CL-1).

30. The antibody of claim 29, comprising a first Fc chain and a second Fc chain, wherein first Fc chain comprises, in N-terminal to C-terminal order, a first hinge region connected to a first CH2 region which is connected to a first CH3 region, and herein the second Fc chain comprises, in N-terminal to C-terminal order, a second hinge region connected to a second CH2 region which is connected to a second CH3 region, and wherein the first hinge region and second hinge region comprise a pair of sequences according to SEQ ID NO: 129 and SEQ ID NO: 131, and the first CH3 region and the second CH3 region comprise either of the following two pairs pair of sequences: SEQ ID NO: 124 and SEQ ID NO: 127; or SEQ ID NO: 147 and SEQ ID NO: 148.

31. The antibody of claim 29, wherein the first CH3 domain and the second CH3 domain each comprise a different and complementary sequence, and the different and complementary sequences are selected from one of the following pairs of different and complementary sequences:
(i) SEQ ID NO: 111 and SEQ ID NO: 106;
(ii) SEQ ID NO: 111 and SEQ ID NO: 114;
(iii) SEQ ID NO: 114 and SEQ ID NO: 117;
(iv) SEQ ID NO: 124 and SEQ ID NO: 127;
(v) SEQ ID NO: 139 and SEQ ID NO: 141; and
(vi) SEQ ID NO: 147 and SEQ ID NO: 148.

32. An antibody comprising an antibody Fc domain comprising a first Fc chain and a second Fc chain, wherein the first Fc chain and the second Fc chain each contain two amino acid modifications that promote the association of the first Fc chain with the second Fc chain, characterized in that
(i) the first Fc chain comprises D(H232)R and K(H440)R, and the second Fc chain comprises D(H232)E and L(H391)E; or
(ii) the first Fc chain comprises D(H232)E and K(H440)R, and the second Fc chain comprises L(H391)R and D(H232)E.

33. The antibody as claimed in claim 16 for use as a medicament.

34. The antibody as claimed in claim 33, wherein the use is for the treatment of one or more selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis, and non-alcoholic steatohepatitis (NASH), and cancer

35. The antibody as claimed in claim 34, wherein the use is for treatment of one or more cancers, and comprises a first anti-cancer therapeutic agent comprising the antibody of any one of claims 1-34, and a second anti-cancer therapeutic agent selected from the group consisting of an anti-OX40 antibody, an anti-4-1BB antibody, an anti-HER2 antibody, a PD-1 pathway antagonist, an anti-PD-1 antibody, an anti-PD-L1 antibody, a TLR3 agonist, a TLR 7/8 agonist, a TLR9 agonist, a bispecific anti-CD47/anti-PD-L1 antibody, and a bispecific anti-P-cadherin/anti-CD3 antibody, sasanlimab, BCD-100, camrelizumab, cemiplimab, genolimzumab, MEDI0680, nivolumab, pembrolizumab, sintilimab, spartalizumab, STI-A1110, tislelizumab, atezolizumab, durvalumab, BMS-936559 (MDX-1105), LY3300054, and TSR-042.

36. A pharmaceutical composition comprising a therapeutically effective amount of the antibody of claim 16 and a pharmaceutically acceptable carrier.

37. The antibody as claimed in claim 19 for use as a medicament.

38. The antibody as claimed in claim 37, wherein the use is for the treatment of one or more selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis, and non-alcoholic steatohepatitis (NASH), and cancer

39. The antibody as claimed in claim 38, wherein the use is for treatment of one or more cancers, and comprises a first anti-cancer therapeutic agent comprising the antibody of any one of claims 1-34, and a second anti-cancer therapeutic agent selected from the group consisting of an anti-OX40 antibody, an anti-4-1BB antibody, an anti-HER2 antibody, a PD-1 pathway antagonist, an anti-PD-1 antibody, an anti-PD-L1 antibody, a TLR3 agonist, a TLR 7/8 agonist, a TLR9 agonist, a bispecific anti-CD47/anti-PD-L1 antibody, and a bispecific anti-P-cadherin/anti-CD3 antibody, sasanlimab, BCD-100, camrelizumab, cemiplimab, genolimzumab, MEDI0680, nivolumab, pembrolizumab, sintilimab, spartalizumab, STI-A1110, tislelizumab, atezolizumab, durvalumab, BMS-936559 (MDX-1105), LY3300054, and TSR-042.

40. A pharmaceutical composition comprising a therapeutically effective amount of the antibody of claim 19 and a pharmaceutically acceptable carrier.

41. The antibody as claimed in claim 27 for use as a medicament.

42. The antibody as claimed in claim 41, wherein the use is for the treatment of one or more selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis, and non-alcoholic steatohepatitis (NASH), and cancer

43. The antibody as claimed in claim 41, wherein the use is for treatment of one or more cancers, and comprises a first anti-cancer therapeutic agent comprising the antibody of any one of claims 1-34, and a second anti-cancer therapeutic agent selected from the group consisting of an anti-OX40 antibody, an anti-4-1BB antibody, an anti-HER2 antibody, a PD-1 pathway antagonist, an anti-PD-1 antibody, an anti-PD-L1 antibody, a TLR3 agonist, a TLR 7/8 agonist, a TLR9 agonist, a bispecific anti-CD47/anti-PD-L1 antibody, and a bispecific anti-P-cadherin/anti-CD3 antibody, sasanlimab, BCD-100, camrelizumab, cemiplimab, genolimzumab, MEDI0680, nivolumab, pembrolizumab, sintilimab, spartalizumab, STI-A1110, tislelizumab, atezolizumab, durvalumab, BMS-936559 (MDX-1105), LY3300054, and TSR-042.

44. A pharmaceutical composition comprising a therapeutically effective amount of the antibody of claim 29 and a pharmaceutically acceptable carrier.

45. A pharmaceutical composition comprising a therapeutically effective amount of the antibody of claim 16 and a pharmaceutically acceptable carrier.

46. A pharmaceutical composition comprising a therapeutically effective amount of the antibody of any one of claim 19 and a pharmaceutically acceptable carrier.

47. A pharmaceutical composition comprising a therapeutically effective amount of the antibody of any one of claim 27 and a pharmaceutically acceptable carrier.

48. A method of treatment of one or more disorders selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis, and non-alcoholic steatohepatitis (NASH), cancer, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL); Heme malignancy; acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), and small lymphocytic lymphoma (SLL); comprising administering to the subject a therapeutic amount of an antibody as claimed in claim 16.

49. A method of treatment of one or more disorders selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis, and non-alcoholic steatohepatitis (NASH), cancer, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL); Heme malignancy; acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), and small lymphocytic lymphoma (SLL); comprising administering to the subject a therapeutic amount of an antibody as claimed in claim 19.

50. A method of treatment of one or more disorders selected from the group consisting of atopic dermatitis, asthma, COPD, food allergy, allergic rhinitis, eosinophilic esophagitis, chronic rhinosinusitis with nasal polyps, alopecia areata, prurigo nodularis, keloids, bullous pemphigoid, chronic urticaria, IPF, scleroderma, systemic sclerosis, and fungal keratitis, and non-alcoholic steatohepatitis (NASH), cancer, bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL); Heme malignancy; acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), and small lymphocytic lymphoma (SLL); comprising administering to the subject a therapeutic amount of an antibody as claimed in claim 27.

51. The method of treatment as claimed in claim 48, wherein the antibody is administered subcutaneously, and optionally, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

52. The method of treatment as claimed in claim 49, wherein the antibody is administered subcutaneously, and optionally, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

53. The method of treatment as claimed in claim 50, wherein the antibody is administered subcutaneously, and optionally, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

54. An isolated nucleic acid selected from the group consisting of:
(i) encoding an IL4-VH, an IL4-VL, or both, of an antibody that binds IL-4, and wherein the nucleic acid comprises: the nucleic acid sequence of SEQ ID NO: 200, the nucleic acid sequence of SEQ ID NO: 201, or both;
(ii) encoding an IL4-VH bearing polypeptide, an IL4-VL bearing polypeptide, or both, of an antibody that binds IL-4, and wherein the nucleic acid comprises: the nucleic acid sequence of SEQ ID NO: 188, the nucleic acid sequence of SEQ ID NO: 189, or both;
(iii) encoding an IL4-VH, an IL4-VL, or both, of an antibody that binds IL-4, and wherein the nucleic acid comprises: the nucleic acid sequence of a sequence encoded by the plasmid deposited at the ATCC and having ATCC Accession No. PTA-127198, and a sequence encoded by the plasmid deposited at the ATCC and having ATCC Accession No. PTA-127197, or both;
(iv) encoding an IL4-VH bearing polypeptide, an IL4-VL bearing polypeptide, or both, of an antibody that binds IL-4, and wherein the nucleic acid comprises: the nucleic acid sequence of a plasmid deposited at the ATCC and having ATCC Accession No. PTA-127192, and a plasmid deposited at the ATCC and having ATCC Accession No. PTA-127194, or both;
(v) encoding an IL13-VH, an IL13-VL, or both, of an antibody that binds IL-13, and wherein the nucleic acid comprises: the nucleic acid sequence of SEQ ID NO: 198, the nucleic acid sequence of SEQ ID NO: 199, or both;
(vi) encoding an IL13-VH bearing polypeptide, an IL13-VL bearing polypeptide, or both, of an antibody that binds IL-13, and wherein the nucleic acid comprises: the nucleic acid sequence of SEQ ID NO: 187, the nucleic acid sequence of SEQ ID NO: 188, or both;
(vii) encoding an IL13-VH, an IL13-VL, or both, of an antibody that binds IL-13, and wherein the nucleic acid comprises: the nucleic acid sequence of a sequence encoded by the plasmid deposited at the ATCC and having ATCC Accession No. PTA-127196, and a sequence encoded by the plasmid deposited at the ATCC and having ATCC Accession No. PTA-127195, or both;
(viii) encoding an IL13-VH bearing polypeptide, an IL13-VL bearing polypeptide, or both, of an antibody that binds IL-13, and wherein the nucleic acid comprises: the nucleic acid sequence of a plasmid deposited at the ATCC and having ATCC Accession No. PTA-127193, and a plasmid deposited at the ATCC and having ATCC Accession No. PTA-127192, or both;
(ix) encoding an IL33-VH, an IL33-VL, or both, of an antibody that binds IL-33, and wherein the nucleic acid comprises: the nucleic acid sequence of SEQ ID NO: 202, the nucleic acid sequence of SEQ ID NO: 203, or both;
(x) encoding an IL33-VH bearing polypeptide, an IL33-VL bearing polypeptide, or both, of an antibody that binds IL-33, and wherein the nucleic acid comprises: the nucleic acid sequence of SEQ ID NO: 190, the nucleic acid sequence of SEQ ID NO: 191, or both;
(xi) encoding an IL33-VH, an IL33-VL, or both, of an antibody that binds IL-33, and wherein the nucleic acid comprises: the nucleic acid sequence of a sequence encoded by the plasmid deposited at the ATCC and having ATCC Accession No. PTA-127210, and a sequence encoded by the plasmid deposited at the ATCC and having ATCC Accession No. PTA-127209, or both;
(xii) encoding an IL33-VH bearing polypeptide, an IL33-VL bearing polypeptide, or both, of an antibody that binds IL-4, and wherein the nucleic acid comprises: the nucleic acid sequence of a plasmid deposited at the ATCC and having ATCC Accession No. PTA-127208, and a plasmid deposited at the ATCC and having ATCC Accession No. PTA-127207, or both;
(xiii) encoding a TSLP-VH, a TSLP-VL, or both, of an antibody that binds TSLP, and wherein the nucleic acid comprises: the nucleic acid sequence of SEQ ID NO: 204, the nucleic acid sequence of SEQ ID NO: 205, or both;
(xiv) encoding a TSLP-VH bearing polypeptide, a TSLP-VL bearing polypeptide, or both, of an antibody that binds TSLP, and wherein the nucleic acid comprises: the nucleic acid sequence of SEQ ID NO: 192, the nucleic acid sequence of SEQ ID NO: 193, or both;
(xv) encoding a TSLP-VH, a TSLP-VL, or both, of an antibody that binds TSLP, and wherein the nucleic acid comprises: the nucleic acid sequence of SEQ ID NO: 204, the nucleic acid sequence of SEQ ID NO: 217, or both;
(xvi) encoding a TSLP-VH, a TSLP-VL, or both, of an antibody that binds TSLP, and wherein the nucleic acid comprises: the nucleic acid sequence of SEQ ID NO: 204, the nucleic acid sequence of SEQ ID NO: 218, or both;
(xvii) encoding a TSLP-VH, a TSLP-VL, or both, of an antibody that binds TSLP, and wherein the nucleic acid comprises: the nucleic acid sequence of a sequence encoded by the plasmid deposited at the ATCC and having ATCC Accession No. PTA-127200, and a sequence encoded by the plasmid deposited at the ATCC and having ATCC Accession No. PTA-127199, or both;
(xviii) encoding a TSLP-VH bearing polypeptide, a TSLP-VL bearing polypeptide, or both, of an antibody that binds TSLP, and wherein the nucleic acid comprises: the nucleic acid sequence of a plasmid deposited at the ATCC and having ATCC Accession No. PTA-127202, and a plasmid deposited at the ATCC and having ATCC Accession No. PTA-127201, or both;
(xix) encoding a p40-VH bearing polypeptide, a p40-VL bearing polypeptide, or both, of an antibody that binds p40, and wherein the nucleic acid comprises: the nucleic acid sequence of SEQ ID NO: 195, the nucleic acid sequence of SEQ ID NO: 194, or both; and
(xx) encoding a p40-VH bearing polypeptide, a p40-VL bearing polypeptide, or both, of an antibody that binds p40, and wherein the nucleic acid comprises: the nucleic acid sequence of a plasmid deposited at the ATCC and having ATCC Accession No. PTA-127206, and a plasmid deposited at the ATCC and having ATCC Accession No. PTA-127205, or both.

55. A vector comprising the polynucleotide of claim 54.

56. An isolated host cell comprising the polynucleotide of any claim 54.

57. A method of producing an isolated antibody, comprising culturing the host cell of claim 56 under conditions that result in production of the antibody, and recovering the antibody.

58. The method of claim 57, wherein the isolated antibody is a heterotrimeric antibody comprising a dual Fab arm and a single Fab arm, wherein the dual Fab arm comprises a first Fab domain connected to a second Fab domain which is connected to a first Fc domain, and the single Fab arm comprises a third Fab domain connected to a second Fc domain, and the method comprising
(i) a first preassembly step wherein the dual Fab arm is incubated in a first preassembly conditioning buffer at a temperature of between 2-10° C. and wherein the pH of the first preassembly conditioning buffer is 1-3 units below the isoelectric point (pI) of the dual Fab arm; and
(ii) a second preassembly step wherein the single Fab arm is incubated in a second preassembly conditioning buffer at a temperature of between 2-10° C. and wherein the pH of the second preassembly conditioning buffer is 1-3 units below the isoelectric point (pI) of the single Fab arm; and
(iii) a third assembly step, wherein the dual Fab arm and single Fab arm from step (i) and (ii) are mixed together in an assembly buffer for between 1-24 hours.
